(19) United States
(12) Patent Application Publication
(10) Pub. No.: US 2022/0031847 A1
(43) Pub. Date: Feb. 3, 2022

(54) THERAPEUTIC METHODS

(71) Applicant: GENEVANT SCIENCES GMBH, Basel (CH)

(72) Inventors: James HEYES, Vancouver (CA); Richard J. HOLLAND, Vancouver (CA); Adam JUDGE, Bainbridge Island, WA (US); Kieu Mong LAM, Richmond (CA)

(73) Assignee: GENEVANT SCIENCES GMBH, Basel (CH)

(21) Appl. No.: 17/290,549

(22) PCT Filed: Nov. 4, 2019
(86) PCT No.: PCT/US19/59711

§ 371 Date: Apr. 30, 2021

Related U.S. Application Data
(60) Provisional application No. 62/755,196, filed on Nov. 2, 2018.

(51) Int. Cl. A61K 47/32; A61K 47/55; A61K 47/34; A61K 47/54

(52) U.S. Cl. CPC A61K 47/32; A61K 47/549; A61K 47/34; A61K 47/551

(57) ABSTRACT

The invention provides methods and compositions for delivering a nucleic acid to a cell or the cytosol of the target cell. The method includes contacting the cell with, 1) a membrane-destabilizing polymer; and 2) a nucleic acid conjugate. The nucleic acid conjugate includes a targeting ligand bound to an optional linker and a nucleic acid.

### CROSS-REFERENCE TO RELATED APPLICATION(S)

This patent application claims the benefit of priority of U.S. application Ser. No. 62/755,196, filed Nov. 2, 2018, which application is herein incorporated by reference.

### BACKGROUND

Targeted nucleic acid conjugates are effective drug delivery systems for biologically active nucleic acids (see WO2017/177326). Drugs based on nucleic acids, which include large nucleic acid molecules such as, e.g., in vitro transcribed messenger RNA (mRNA) as well as smaller polynucleotides that interact with a messenger RNA or a gene, have to be delivered to the proper cellular compartment in order to be effective.

For example, double-stranded nucleic acids such as double-stranded RNA molecules (dsRNA), including, e.g., siRNAs, suffer from their physico-chemical properties that render them impermeable to cells. Upon delivery into the proper compartment. siRNAs block gene expression through a highly conserved regulatory mechanism known as RNA interference (RNAi). Typically, siRNAs are large in size with a molecular weight ranging from 12-17 kDa, and are highly anionic due to their phosphate backbone with up to 50 negative charges. In addition, the two complementary RNA strands result in a rigid helix. These features contribute to the siRNA's poor “drug-like” properties. When administered intravenously, the siRNA is rapidly excreted from the body with a typical half-life in the range of only 10 minutes. Additionally. siRNAs are rapidly degraded by nucleases present in blood and other fluids or in tissues, and have been shown to stimulate strong immune responses in vitro and in vivo. See, e.g., Robbins et al., Oligonucleotides 19:89-102, 2009. mRNA molecules suffer from similar issues of impermeability, fragility, and immunogenicity.

By introduction of appropriate chemical modifications, stability towards nucleases can be increased and at the same time immune stimulation can be suppressed. Conjugation of certain ligands to siRNAs can improve the pharmacokinetic characteristics of the double-stranded RNA molecule. It has been demonstrated that certain small molecule siRNA conjugates are efficacious in a specific down regulation of a gene expressed in hepatocytes of rodents. However, in order to elicit the desired biologic effect, a large dose is needed. See Soutschek et al, Nature 432. 173-178, 2004.

Despites previous efforts, improved methods for delivering nucleic acids into cells are needed. For example, there is a need for methods that improve the potency, reduce the required dose, and/or reduce the dosing frequency Methods for and formulations that can be used to deliver nucleic acids subcutaneous are also needed.

### BRIEF SUMMARY

In one embodiment the invention provides a method for delivering a nucleic acid to a cell comprising contacting the cell with, 1) a membrane-destabilizing polymer; and 2) a nucleic acid conjugate of Formula (X):

A-B-C (X)

wherein A is a targeting ligand, B is an optional linker, and C is a nucleic acid.

In one embodiment the invention provides a method for delivering a nucleic acid to the cytosol of a target cell within a subject, the method comprising. administering to the subject (a) a membrane-destabilizing polymer, and (b) a nucleic acid conjugate of Formula (X):

A-B-C (X)

wherein A is a targeting ligand, B is an optional linker, and C is a nucleic acid, wherein the nucleic acid is delivered to the cytosol of the target cell.

In one embodiment the invention provides a method comprising administering to an animal, 1) a membrane-destabilizing polymer; and 2) a nucleic acid conjugate of Formula (X):

A-B-C (X)

wherein A is a targeting ligand, B is an optional linker, and C is a nucleic acid.

In one embodiment the invention provides a composition comprising: a) a pharmaceutically acceptable carrier, b) a membrane-destabilizing polymer; and c) a nucleic acid conjugate of Formula (X):

A-B-C (X)

wherein A is a targeting ligand, B is an optional linker, and C is a nucleic acid. In one embodiment the composition is formulated for administration by injection. In one embodiment the composition is formulated for administration by subcutaneous injection.

In one embodiment the invention provides a method for treating a disease characterized by overexpression of a polypeptide, comprising administering to an animal having the disease a therapeutically effective amount of (a) a membrane-destabilizing polymer; and b) a nucleic acid conjugate of Formula (X):

A-B-C (X)

wherein A is a targeting ligand, B is an optional linker, and C is an siRNA that targets expression of the overexpressed polypeptide.

In one embodiment the invention provides a method to deliver an siRNA to the liver of an animal, comprising administering to the animal, (a) a membrane-destabilizing polymer that comprises a targeting moiety (T⁵) selected to promote hepatocyte-specific delivery of the polymer; and b) a nucleic acid conjugate of Formula (X):

A-B-C (X)

wherein A is a targeting ligand, B is an optional linker, and C is the siRNA.

In one embodiment the invention provides a method to treat a hepatitis B viral infection in an animal, comprising administering to the animal: (a) a membrane-destabilizing polymer, comprising a targeting moiety (T⁵) selected to promote hepatocyte-specific delivery of the polymer, and (b) a nucleic acid conjugate of formula (X):

A-B-C (X)

wherein A is a targeting ligand selected to promote hepatocyte-specific delivery of the conjugate, B is an optional linker, and C is an siRNA that is effective to treat the hepatitis B viral infection.

In one embodiment the invention provides a kit comprising: 1) a membrane-destabilizing polymer; 2) a nucleic acid conjugate of Formula (X):

A-B-C (X)

wherein A is a targeting ligand, B is an optional linker, and C is a nucleic acid; and 3) instructions for delivering a nucleic acid to a cell comprising contacting the cell with the nucleic acid conjugate and the membrane-destabilizing polymer.

In one embodiment the invention provides a kit comprising: 1) a membrane-destabilizing polymer; 2) a nucleic acid conjugate of Formula (X):

A-B-C (X)

wherein A is a targeting ligand, B is an optional linker, and C is a nucleic acid; and 3) instructions for delivering a nucleic acid to the cytosol of a target cell within a subject by administering the nucleic acid conjugate and the membrane-destabilizing polymer to the subject.

In one embodiment the invention provides a kit comprising: 1) a membrane-destabilizing polymer; 2) a nucleic acid conjugate of Formula (X):

A-B-C (X)

wherein A is a targeting ligand, B is an optional linker, and C is a nucleic acid; and 3) instructions for administering the nucleic acid conjugate and the membrane-destabilizing polymer to an animal.

In one embodiment the invention provides a membrane-destabilizing polymer and a nucleic acid conjugate of Formula (X):

A-B-C (X)

wherein A is a targeting ligand, B is an optional linker, and C is a nucleic acid; for use in medical therapy.

In one embodiment the invention provides a nucleic acid conjugate of Formula (X):

A-B-C (X)

wherein A is a targeting ligand, B is an optional linker, and C is a nucleic acid; for the prophylactic or therapeutic treatment of a disease treatable with the nucleic acid, in combination with a membrane-destabilizing polymer.

In one embodiment the invention provides the use of a nucleic acid conjugate of Formula (X):

A-B-C (X)

wherein A is a targeting ligand, B is an optional linker, and C is a nucleic acid; to prepare a medicament for treating a disease treatable with the nucleic acid, in combination with a membrane-destabilizing polymer.

In one embodiment the invention provides a nucleic acid conjugate of Formula (X):

A-B-C (X)

wherein A is a targeting ligand, B is an optional linker, and C is a nucleic acid, wherein the nucleic acid conjugate is associated non-covalently with a membrane-destabilizing polymer.

In one embodiment the invention provides a nucleic acid conjugate of Formula (X):

A-B-C (X)

wherein A is a targeting ligand, B is an optional linker, and C is a nucleic acid, wherein the nucleic acid conjugate is partially or fully encapsulated by a micelle that comprises a plurality of membrane-destabilizing polymers.

In one embodiment the invention provides a nucleic acid conjugate of Formula (X):

A-B-C (X)

wherein A is a targeting ligand, B is an optional linker, and C is a nucleic acid, wherein the nucleic acid conjugate is partially encapsulated by a micelle that comprises a plurality of membrane-destabilizing polymers.

In one embodiment the invention provides a nucleic acid conjugate of Formula (X):

A-B-C (X)

wherein A is a targeting ligand, B is an optional linker, and C is a nucleic acid, wherein the nucleic acid conjugate is fully encapsulated by a micelle that comprises a plurality of membrane-destabilizing polymers.

In one embodiment the invention provides a pharmaceutical composition comprising a pharmaceutically acceptable carrier, and a nucleic acid conjugate of Formula (X):

A-B-C (X)

wherein A is a targeting ligand, B is an optional linker, and C is a nucleic acid, wherein the nucleic acid conjugate is partially or fully encapsulated by a micelle that comprises a plurality of membrane-destabilizing polymers.

In one embodiment, the invention provides compounds, compositions, and methods that can be used to target delivery of therapeutic nucleic acids (e.g. to the liver). Specifically, it includes the use of a polymer micelle as a potency enhancer to a subcutaneously-administered conjugate platform for targeted delivery of nucleci acid therapeutics to the liver. The polymer micelles typically remain intact during delivery to hepatocytes and exert their functionality, for example, when administered subcutaneously. Gene silencing is examined by measuring the inhibition or reduction in expression of the target gene relative to the vehicle control.

Favorable results were obtained in mice, where co-administration of a membrane-destabilizing polymer and a nucleic acid conjugate enhanced potency by about 5-fold; a more rapid onset of action and a longer duration of effect were also seen.

Other objects, features, and advantages of the present invention will be apparent to one of skill in the art from the following detailed description and figures.

### DETAILED DESCRIPTION

### Membrane Destabilizing Polymers

Membrane destabilizing polymers are reported in United States Patent Application Publication Numbers: US2010/0160216, US2010/0210504, US2011/0143434, US2011/0123636, US2016/0250338, US2017/0239360, and US2016/0206750, and in International Patent Application Publication Numbers: WO2009/140427, WO2009/140429, WO2015/017519, and WO2016/118697. Additionally, descriptions of the synthesis of certain specific membrane-destabilizing polymers can be found in the supplemental section of Prieve et al., Mol. Ther., 2018, 26, 3.

In one embodiment, the membrane destabilizing polymer comprises three distinct regions:

First, hepatocyte targeting can be achieved with a targeting moiety such as a single N-Acetylgalactoseamine monosaccharide unit that interacts with one of the three trivalent domains of the ASGPr receptor which is highly expressed on the surface of hepatocytes. This monosaccharide unit forms the “head’ of the polymer chain. The N-acetyl galactose amine (GalNAc or NAG) can be attached to the second functional domain of the polymer via a PEG12 amino acid spacer coupled to ethyl carbonotrithioate (ECT). This represents the starting “chain transfer agent” or CTA. Subsequent polymerization reactions can take place on the fully deprotected monosaccharide.

The second “solubilizing” or hydrophilic region is comprised of polyethyleneglycol methacrylate 4-5 (PEGMA 4-5) The number 4 and 5 refers to the number of ethylene glycol repeats in the monomer) and hydroxyethyl methacrylate (HMA). Usually in a ratio around 75/25 PEGMA/HMA. The polymerization can occur using reversible addition-fragmentation chain transfer (RAFT) which allows control over the generated molecular weight and polydispersity during a free-radical polymerization initiated with azobisisobutyronitrile (AIBN). The reaction can proceed at a fixed time at a certain concentration and temperature to produce a hydrophilic polymer around 4 kDa capped with a terminal trithiocarbonate functionality that allows further polymerization.

The third region of the polymer provides the endosomal release functionality. It can also be synthesized using RAFT polymersiation, however in this case the monomeric units in the reaction are dimethylaminoethyl acrylate (DMAEA), butyl methyacrylate (BMA) and propylacrylic acid (PAA) (typically in ratios of about 33%/55%/12%). This second polymerization step extends the polymer out by around another 5 kDa. Following polymerization, the polymer end group (trithiocarbonate) can be removed by radical induced reduction and the final polymer characterized by 1H NMR, HPLC and GPC (to determine MW and polydispersity)

The combination of the two polymeric regions helps maximize efficacy. At physiological or neutral pH the polymer is typically neutral. Moreover at neutral pH the second endosomal release region displays hydrophobic character. In conjugation with the hydrophilic domain, if the polymer is above the critical micelle concentration (CMC) in aqueous media, small micelle structures will spontaneously form. These have been shown to have pH responsive membrane destabilizing activity in red blood cell hemolysis assays: below the CMC, hemolysis drops off precipitously. During endocytosis and subsequent decrease in pH, the polymer can become positively charged and consequently promote endosomal release.

In one embodiment, the a membrane-destabilizing polymer is a polymer of formula (XX):

T⁵-L-[PEGMAₘ-M²ₙ]ᵥ-[DMAEMA_{q}-PAAᵣ-BMAₛ]_{w} (XX)

wherein:

PEGMA is polyethyleneglycol methacrylate residue with 2-20 ethylene glycol units;

M² is a methacrylate residue selected from the group consisting of
a (C₄-C₁₈)alkyl-methacrylate residue;
a (C₄-C₁₈) branched alkyl-methacrylate residue;
a cholesteryl methacrylate residue;
a (C₄-C₁₈)alkyl-methacrylate residue substituted with one or more fluorine atoms; and
a (C₄-C₁₈) branched alkyl-methacrylate residue substituted with one or more fluorine atoms;

BMA is butyl methacrylate residue;

PAA is propyl acrylic acid residue;

DMAEMA is dimethylaminoethyl methacrylate residue;

m and n are each a mole fraction greater than 0, wherein m is greater than n and m+n=1;

q is a mole fraction of 0.2 to 0.75;

r is a mole fraction of 0.05 to 0.6;

s is a mole fraction of 0.2 to 0.75;

q+r+s=1;

v is 1 to 25 kDa;

w is 1 to 25 kDa;

T⁵ is a targeting moiety (e.g., a peptide, polymer or saccharide); and

L is absent or is a linking moiety.

In one embodiment, M² is selected from the group consisting of:
2,2,3,3,4,4,4-heptafluorobutyl methacrylate residue,
3,3,4,4,5,6,6,6-octafluoro-5(trifluoromethyl)hexyl methacrylate residue,
2,2,3,3,4,4,5,5,6,6,7,7,8,8,8-pentadecafluorooctyl 2-methylacrylate residue,
3,3,4,4,5,5,6,6,6-nonafluorohexyl methacrylate residue,
3,3,4,4,5,5,6,6,7,7,8,8,8-tridecafluorooctyl methacrylate residue,
1,1,1-trifluoro-2-(trifluoromethyl)-2-hydroxy-4-methyl-5-pentyl methacrylate residue, 2-[(1′,1′, 1′-trifluoro-2′-(trifluoro methyl)-2′-hydroxy)propyl]-3-norbornyl methacrylate residue, 2-ethylhexyl methacrylate residue,
butyl methacrylate residue,
hexyl methacrylate residue,
octyl methacrylate residue,
n-decyl methacrylate residue,
lauryl methacrylate residue,
myristyl methacrylate residue,
stearyl methacrylate residue,
cholesteryl methacrylate residue,
ethylene glycol phenyl ether methacrylate residue,
2-propenoic acid, 2-methyl-, 2-phenylethyl ester residue,
2-propenoic acid, 2-methyl-, 2-[[(1,1-dimethylethoxy)carbonyl]amino]ethyl ester residue,
2-propenoic acid, 2-methyl-, 2-(1H-imidazol-1-yl)ethyl ester residue,
2-propenoic acid, 2-methyl-, cyclohexyl ester residue,
2-propenoic acid, 2-methyl-, 2-[bis(1-methylethyl)amino]ethyl ester residue,
2-propenoic acid, 2-methyl-, 3-methylbutyl ester residue,
neopentyl methacrylate residue,
tert-butyl methacrylate residue,
3,3,5-trimethyl cyclohexyl methacrylate residue,
2-hydroxypropyl methacrylate residue,
5-nonyl methacrylate residue,
2-butyl-1-octyl methacrylate residue,
2-hexyl-1-decyl methacrylate residue, and
2-(tert-butyl amino)ethyl methacrylate residue.

Targeting moiety T⁵ is a moiety that can be, e.g., a peptide, polymer or saccharide. The targeting moiety T⁵ in certain embodiments targets delivery to a location in the body, e.g., targets delivery to a specific organ or cell type. In certain embodiments, T⁵ is a peptide. In certain embodiments, T⁵ is a polymer. In certain embodiments, T is a saccharide.

In one embodiment, the a membrane-destabilizing polymer is a polymer of formula (XXI):

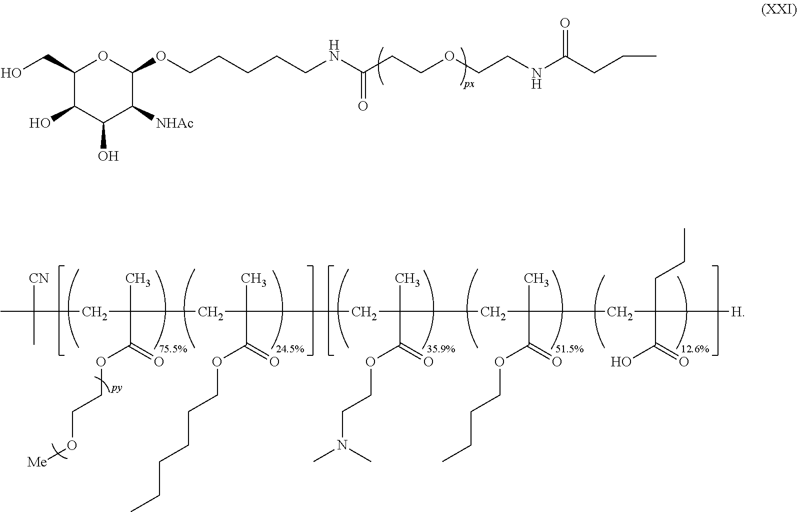

In some embodiments, px is an integer of from about 2 to about 50, e.g., from about 2 to about 20, e.g., from 4 to 12 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50). In some embodiments, px is an integer of from about 8 to about 16 (e.g., 8, 9, 10, 11, 12, 13, 14, 15, or 16). In some embodiments, px is about 12. In some embodiments, py is an integer of from about 2 to about 20 (e.g., 2, 3, 4, 5, 6, 7, 8, 9, 10, 11, 12, 13, 14, 15, 16, 17, 18, 19, or 20). In some embodiments, py is an integer of from about 2 to about 10 (e.g., 2, 3, 4, 5, 6, 7, 8, 9, or 10). In some embodiments, py is an integer of from about 4 to about 5 (e.g., 4 or 5).

In a polymer of formula (X), it should be understood that the representation of the polymer block:

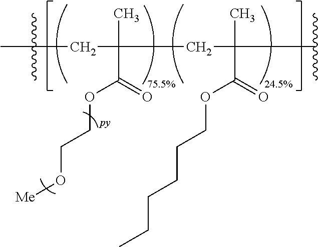

designates a polymer block with the two monomer groups a and b:

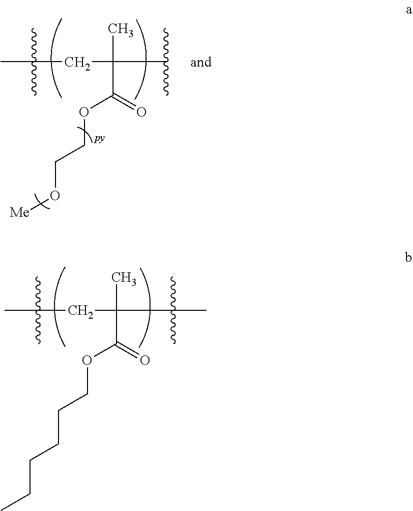

distributed throughout the block, wherein about 75.5 weight percent of the block is monomer group a and about 24.5 weight percent of the block is monomer group b. The representation of the polymer block:

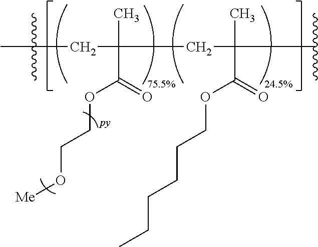

does not designate a polymer block comprising one homo-polymer block of monomer group a and one homo-polymer block of monomer group b. The same is true for the representation of the polymer block:

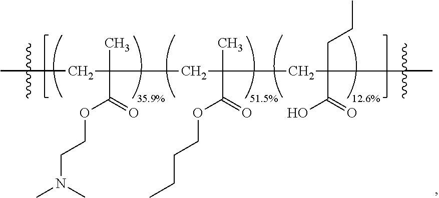

which has three monomer units distributed throughout the block, in approximately the total weight ratios shown.

### Targeted Nucleic Acid Conjugates

The terms “alkoxy,” and “alkylthio”, are used in their conventional sense, and refer to those alkyl groups attached to the remainder of the molecule via an oxygen atom (“oxy”) or thio group, and further include mono- and poly-halogenated variants thereof.

The term “alkyl”, by itself or as part of another substituent, means, unless otherwise stated, a straight or branched chain hydrocarbon radical, having the number of carbon atoms designated (i.e., C₁₋₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. The term “alkenyl” refers to an unsaturated alkyl radical having one or more double bonds. Similarly, the term “alkynyl” refers to an unsaturated alkyl radical having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “animal” includes mammalian species, such as a human, mouse, rat, dog, cat, hamster, guinea pig, rabbit, livestock, and the like.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (e.g., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “gene” refers to a nucleic acid (e.g., DNA or RNA) sequence that comprises partial length or entire length coding sequences necessary for the production of a polypeptide or precursor polypeptide.

“Gene product,” as used herein, refers to a product of a gene such as an RNA transcript or a polypeptide.

The terms “halo” or “halogen” mean, unless otherwise stated, a fluorine, chlorine, bromine, or iodine atom.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl and furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “saccharide” includes monosaccharides, disaccharides and trisaccharides, all of which can be optionally substituted. The term includes glucose, sucrose fructose, galactose and ribose, as well as deoxy sugars such as deoxyribose and amino sugar such as galactosamine. Saccharide derivatives can conveniently be prepared as described in International Patent Applications Publication Numbers WO 96/34005 and 97/03995. A saccharide can conveniently be linked to the remainder of a compound of formula I through an ether bond, a thioether bond (e.g. an S-glycoside), an amine nitrogen (e.g., an N-glycoside), or a carbon-carbon bond (e.g. a C-glycoside). In one embodiment the saccharide can conveniently be linked to the remainder of a compound of formula I through an ether bond.

The term “small-interfering RNA” or “siRNA” as used herein refers to double stranded RNA (i.e., duplex RNA) that is capable of reducing or inhibiting the expression of a target gene or sequence (e.g., by mediating the degradation or inhibiting the translation of mRNAs which are complementary to the siRNA sequence) when the siRNA is in the same cell as the target gene or sequence. The siRNA may have substantial or complete identity to the target gene or sequence, or may comprise a region of mismatch (i.e., a mismatch motif). In certain embodiments, the siRNAs may be about 19-25 (duplex) nucleotides in length, and is preferably about 20-24, 21-22, or 21-23 (duplex) nucleotides in length. siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand.

In certain embodiments, the 5′ and/or 3′ overhang on one or both strands of the siRNA comprises 1-4 (e.g., 1, 2, 3, or 4) modified and/or unmodified deoxythymidine (t or dT) nucleotides, 1-4 (e.g., 1, 2, 3, or 4) modified (e.g., 2′OMe) and/or unmodified uridine (U) ribonucleotides, and/or 1-4 (e.g., 1, 2, 3, or 4) modified (e.g., 2′OMe) and/or unmodified ribonucleotides or deoxyribonucleotides having complementarity to the target sequence (e.g., 3′overhang in the antisense strand) or the complementary strand thereof (e.g., 3′ overhang in the sense strand).

Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

The phrase “inhibiting expression of a target gene” refers to the ability of a siRNA of the invention to silence, reduce, or inhibit expression of a target gene. To examine the extent of gene silencing, a test sample (e.g., a biological sample from an organism of interest expressing the target gene or a sample of cells in culture expressing the target gene) is contacted with a siRNA that silences, reduces, or inhibits expression of the target gene. Expression of the target gene in the test sample is compared to expression of the target gene in a control sample (e.g., a biological sample from an organism of interest expressing the target gene or a sample of cells in culture expressing the target gene) that is not contacted with the siRNA. Control samples (e.g., samples expressing the target gene) may be assigned a value of 100%. In particular embodiments, silencing, inhibition, or reduction of expression of a target gene is achieved when the value of the test sample relative to the control sample (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays include, without limitation, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

An “effective amount” or “therapeutically effective amount” of a therapeutic nucleic acid such as siRNA is an amount sufficient to produce the desired effect, e.g., an inhibition of expression of a target sequence in comparison to the normal expression level detected in the absence of a siRNA. In particular embodiments, inhibition of expression of a target gene or target sequence is achieved when the value obtained with a siRNA relative to the control (e.g., buffer only, an siRNA sequence that targets a different gene, a scrambled siRNA sequence, etc.) is about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 70%, 65%, 60%, 55%, 50%, 45%, 40%, 35%, 30%, 25%, 20%, 15%, 10%, 5%, or 0%. Suitable assays for measuring the expression of a target gene or target sequence include, but are not limited to, examination of protein or mRNA levels using techniques known to those of skill in the art, such as, e.g., dot blots, Northern blots, in situ hybridization, ELISA, immunoprecipitation, enzyme function, as well as phenotypic assays known to those of skill in the art.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs). Additionally, nucleic acids can include one or more UNA moieties.

The term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl, silyl and 2,2-dimethoxy propene. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

The term “synthetic activating group” refers to a group that can be attached to an atom to activate that atom to allow it to form a covalent bond with another reactive group. It is understood that the nature of the synthetic activating group may depend on the atom that it is activating. For example, when the synthetic activating group is attached to an oxygen atom, the synthetic activating group is a group that will activate that oxygen atom to form a bond (e.g. an ester, carbamate, or ether bond) with another reactive group. Such synthetic activating groups are known. Examples of synthetic activating groups that can be attached to an oxygen atom include, but are not limited to, acetate, succinate, triflate, and mesylate. When the synthetic activating group is attached to an oxygen atom of a carboxylic acid, the synthetic activating group can be a group that is derivable from a known coupling reagent (e.g. a known amide coupling reagent). Such coupling reagents are known. Examples of such coupling reagents include, but are not limited to, N,N′-Dicyclohexylcarbodimide (DCC), hydroxybenzotriazole (HOBt), N-(3-Dimethylaminopropyl)-N′-ethylcarbonate (EDC), (Benzotriazol-1-yloxy)tris(dimethylamino)phosphonium hexafluorophosphate (BOP), benzotriazol-1-yl-oxytripyrrolidinophosphonium hexafluorophosphate (PyBOP), (1-[Bis(dimethylamino)methylene]-1H-1,2,3-triazolo[4,5-b]pyridinium 3-oxid hexafluorophosphate (HATU), propylphosphonic anhydride solution (T3P) or O-benzotriazol-1-yl-N,N,N′,N′-tetramethyluronium hexafluorophosphate (HBTU).

### Nucleic Acids

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. RNA may be in the form, for example, of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), self-amplifying RNA (sa-RNA), and combinations thereof. Accordingly, in the context of this invention, the terms “polynucleotide” and “oligonucleotide” refer to a polymer or oligomer of nucleotide or nucleoside monomers consisting of naturally-occurring bases, sugars and intersugar (backbone) linkages. The terms “polynucleotide” and “oligonucleotide” also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

As described herein, certain embodiments of the invention provide methods and compositions for delivering a nucleic acid to a cell. In certain embodiments, the nucleic acid is a nucleic acid described herein. For example, the nucleic acids used herein can be single-stranded DNA or RNA, or double-stranded DNA or RNA, or DNA-RNA hybrids. Examples of double-stranded RNA are described herein and include, e.g., siRNA and other RNAi agents such as aiRNA and pre-miRNA. Single-stranded nucleic acids include, e.g., antisense oligonucleotides, ribozymes, mature miRNA, and triplex-forming oligonucleotides.

In certain embodiments, the nucleic acid is an oligonucleotide. In particular embodiments, the oligonucleotide ranges from about 10 to about 100 nucleotides in length. In various related embodiments, oligonucleotides, both single-stranded, double-stranded, and triple-stranded, may range in length from about 10 to about 60 nucleotides, from about 15 to about 60 nucleotides, from about 20 to about 50 nucleotides, from about 15 to about 30 nucleotides, or from about 20 to about 30 nucleotides in length.

In certain embodiments, the nucleic acid is selected from the group consisting of small interfering RNA (siRNA), Dicer-substrate dsRNA, small hairpin RNA (shRNA), asymmetrical interfering RNA (aiRNA), microRNA (miRNA), tRNA, rRNA, tRNA, viral RNA (vRNA), self-amplifying RNA (sa-RNA), and combinations thereof.

In certain embodiments, the nucleic acid is an antisense molecule. In certain embodiments, the nucleic acid is a miRNA molecule. In certain embodiments, the nucleic acid is a siRNA. Suitable siRNA, as well as method and intermediates useful for their preparation are reported in International Patent Application Publication Number WO2016/054421.

Target Genes

In certain embodiments, the nucleic acid (e.g., siRNA) may be used to downregulate or silence the translation (i.e., expression) of a gene of interest. Genes of interest include, but are not limited to, genes associated with viral infection and survival, genes associated with metabolic diseases and disorders (e.g., liver diseases and disorders), genes associated with tumorigenesis and cell transformation (e.g., cancer), angiogenic genes, immunomodulator genes such as those associated with inflammatory and autoimmune responses, ligand receptor genes, and genes associated with neurodegenerative disorders. In certain embodiments, the gene of interest is expressed in hepatocytes.

Genes associated with viral infection and survival include those expressed by a virus in order to bind, enter, and replicate in a cell. Of particular interest are viral sequences associated with chronic viral diseases. Viral sequences of particular interest include sequences of Filoviruses such as Ebola virus and Marburg virus (see, e.g., Geisbert et al., J. Infect. Dis., 193:1650-1657 (2006)); Arenaviruses such as Lassa virus, Junin virus, Machupo virus, Guanarito virus, and Sabia virus (Buchmeier et al., Arenaviridae: the viruses and their replication, In: FIELDS VIROLOGY, Knipe et al. (eds.), 4th ed., Lippincott-Raven, Philadelphia, (2001)); Influenza viruses such as Influenza A, B, and C viruses, (see, e.g., Steinhauer et al., Annu Rev Genet., 36:305-332 (2002); and Neumann et al., J Gen Virol., 83:2635-2662 (2002)); Hepatitis viruses (see, e.g., Hamasaki et al., FEBS Lett., 543:51 (2003); Yokota et al., EMBO Rep., 4:602 (2003); Schlomai et al., Hepatology, 37:764 (2003); Wilson et al., Proc. Natl. Acad. Sci. USA, 100:2783 (2003); Kapadia et al., Proc. Natl. Acad. Sci. USA, 100:2014 (2003); and FIELDS VIROLOGY, Knipe et al. (eds.), 4th ed., Lippincott-Raven, Philadelphia (2001)); Human Immunodeficiency Virus (HIV) (Banerjea et al., Mol. Ther., 8:62 (2003); Song et al., J. Virol., 77:7174 (2003); Stephenson, JAMA, 289:1494 (2003); Qin et al., Proc. Natl. Acad. Sci. USA, 100:183 (2003)); Herpes viruses (Jia et al., J. Virol., 77:3301 (2003)); and Human Papilloma Viruses (HPV) (Hall et al., J. Virol., 77:6066 (2003); Jiang et al., Oncogene, 21:6041 (2002)).

Exemplary Filovirus nucleic acid sequences that can be silenced include, but are not limited to, nucleic acid sequences encoding structural proteins (e.g., VP30, VP35, nucleoprotein (NP), polymerase protein (L-pol)) and membrane-associated proteins (e.g., VP40, glycoprotein (GP), VP24). Complete genome sequences for Ebola virus are set forth in, e.g., Genbank Accession Nos. NC_002549; AY769362; NC_006432; NC_004161; AY729654; AY354458; AY142960; AB050936; AF522874; AF499101; AF272001; and AF086833. Ebola virus VP24 sequences are set forth in, e.g., Genbank Accession Nos. U77385 and AY058897. Ebola virus L-pol sequences are set forth in, e.g., Genbank Accession No. X67110. Ebola virus VP40 sequences are set forth in, e.g., Genbank Accession No. AY058896. Ebola virus NP sequences are set forth in, e.g., Genbank Accession No. AY058895. Ebola virus GP sequences are set forth in, e.g., Genbank Accession No. AY058898; Sanchez et al., Virus Res., 29:215-240 (1993); Will et al., J. Virol., 67:1203-1210 (1993); Volchkov et al., FEBS Lett., 305:181-184 (1992); and U.S. Pat. No. 6,713,069.

Additional Ebola virus sequences are set forth in, e.g., Genbank Accession Nos. L11365 and X61274. Complete genome sequences for Marburg virus are set forth in, e.g., Genbank Accession Nos. NC_001608; AY430365; AY430366; and AY358025. Marburg virus GP sequences are set forth in, e.g., Genbank Accession Nos. AF005734; AF005733; and AF005732. Marburg virus VP35 sequences are set forth in, e.g., Genbank Accession Nos. AF005731 and AF005730. Additional Marburg virus sequences are set forth in, e.g., Genbank Accession Nos. X64406; Z29337; AF005735; and Z12132. Non-limiting examples of siRNA molecules targeting Ebola virus and Marburg virus nucleic acid sequences include those described in U.S. Patent Publication No. 20070135370, the disclosure of which is herein incorporated by reference in its entirety for all purposes.

Exemplary Influenza virus nucleic acid sequences that can be silenced include, but are not limited to, nucleic acid sequences encoding nucleoprotein (NP), matrix proteins (M1 and M2), nonstructural proteins (NS1 and NS2), RNA polymerase (PA, PB1, PB2), neuraminidase (NA), and haemagglutinin (HA). Influenza A NP sequences are set forth in, e.g., Genbank Accession Nos. NC_004522; AY818138; AB166863; AB188817; AB189046; AB189054; AB189062; AY646169; AY646177; AY651486; AY651493; AY651494; AY651495; AY651496; AY651497; AY651498; AY651499; AY651500; AY651501; AY651502; AY651503; AY651504; AY651505; AY651506; AY651507; AY651509; AY651528; AY770996; AY790308; AY818138; and AY818140. Influenza A PA sequences are set forth in, e.g., Genbank Accession Nos. AY818132; AY790280; AY646171; AY818132; AY818133; AY646179; AY818134; AY551934; AY651613; AY651610; AY651620; AY651617; AY651600; AY651611; AY651606; AY651618; AY651608; AY651607; AY651605; AY651609; AY651615; AY651616; AY651640; AY651614; AY651612; AY651621; AY651619; AY770995; and AY724786. Non-limiting examples of siRNA molecules targeting Influenza virus nucleic acid sequences include those described in U.S. Patent Publication No. 20070218122, the disclosure of which is herein incorporated by reference in its entirety for all purposes.

Exemplary hepatitis virus nucleic acid sequences that can be silenced include, but are not limited to, nucleic acid sequences involved in transcription and translation (e.g., En1, En2, X, P) and nucleic acid sequences encoding structural proteins (e.g., core proteins including C and C-related proteins, capsid and envelope proteins including S, M, and/or L proteins, or fragments thereof) (see, e.g., FIELDS VIROLOGY, supra). Exemplary Hepatitis C virus (HCV) nucleic acid sequences that can be silenced include, but are not limited to, the 5′-untranslated region (5′-UTR), the 3′-untranslated region (3′-UTR), the polyprotein translation initiation codon region, the internal ribosome entry site (IRES) sequence, and/or nucleic acid sequences encoding the core protein, the E1 protein, the E2 protein, the p7 protein, the NS2 protein, the NS3 protease/helicase, the NS4A protein, the NS4B protein, the NS5A protein, and/or the NS5B RNA-dependent RNA polymerase. HCV genome sequences are set forth in, e.g., Genbank Accession Nos. NC_004102 (HCV genotype 1a), AJ238799 (HCV genotype 1b), NC_009823 (HCV genotype 2), NC_009824 (HCV genotype 3), NC_009825 (HCV genotype 4), NC_009826 (HCV genotype 5), and NC_009827 (HCV genotype 6). Hepatitis A virus nucleic acid sequences are set forth in, e.g., Genbank Accession No. NC_001489; Hepatitis B virus nucleic acid sequences are set forth in, e.g., Genbank Accession No. NC-003977; Hepatitis D virus nucleic acid sequence are set forth in, e.g., Genbank Accession No. NC_001653; Hepatitis E virus nucleic acid sequences are set forth in, e.g., Genbank Accession No. NC_001434; and Hepatitis G virus nucleic acid sequences are set forth in, e.g., Genbank Accession No. NC_001710. Silencing of sequences that encode genes associated with viral infection and survival can conveniently be used in combination with the administration of conventional agents used to treat the viral condition. Non-limiting examples of siRNA molecules targeting hepatitis virus nucleic acid sequences include those described in U.S. Patent Publication Nos. 20060281175, 20050058982, and 20070149470; U.S. Pat. No. 7,348,314; and U.S. Provisional Application No. 61/162,127, filed Mar. 20, 2009, the disclosures of which are herein incorporated by reference in their entirety for all purposes.

Genes associated with metabolic diseases and disorders (e.g., disorders in which the liver is the target and liver diseases and disorders) include, for example, genes expressed in dyslipidemia (e.g., liver X receptors such as LXRα and LXRβ (Genback Accession No. NM-007121), farnesoid X receptors (FXR) (Genbank Accession No. NM_005123), sterol-regulatory element binding protein (SREBP), site-1 protease (SIP), 3-hydroxy-3-methylglutaryl coenzyme-A reductase (HMG coenzyme-A reductase), apolipoprotein B (ApoB) (Genbank Accession No. NM_000384), apolipoprotein CIII (ApoC3) (Genbank Accession Nos. NM_000040 and NG-008949 REGION: 5001.8164), and apolipoprotein E (ApoE) (Genbank Accession Nos. NM_000041 and NG-007084 REGION: 5001.8612)); and diabetes (e.g., glucose 6-phosphatase) (see, e.g., Forman et al., Cell, 81:687 (1995); Seol et al., Mol. Endocrinol., 9:72 (1995), Zavacki et al., Proc. Natl. Acad. Sci. USA, 94:7909 (1997); Sakai et al., Cell, 85:1037-1046 (1996); Duncan et al., J. Biol. Chem., 272:12778-12785 (1997); Willy et al., Genes Dev., 9:1033-1045 (1995); Lehmann et al., J. Biol. Chem., 272:3137-3140 (1997); Janowski et al., Nature, 383:728-731 (1996); and Peet et al., Cell, 93:693-704 (1998)). One of skill in the art will appreciate that genes associated with metabolic diseases and disorders (e.g., diseases and disorders in which the liver is a target and liver diseases and disorders) include genes that are expressed in the liver itself as well as and genes expressed in other organs and tissues. Silencing of sequences that encode genes associated with metabolic diseases and disorders can conveniently be used in combination with the administration of conventional agents used to treat the disease or disorder. Non-limiting examples of siRNA molecules targeting the ApoB gene include those described in U.S. Patent Publication No. 20060134189, the disclosure of which is herein incorporated by reference in its entirety for all purposes. Non-limiting examples of siRNA molecules targeting the ApoC3 gene include those described in U.S. Provisional Application No. 61/147,235, filed Jan. 26, 2009, the disclosure of which is herein incorporated by reference in its entirety for all purposes.

Examples of gene sequences associated with tumorigenesis and cell transformation (e.g., cancer or other neoplasia) include mitotic kinesins such as Eg5 (KSP, KIF11; Genbank Accession No. NM_004523); serine/threonine kinases such as polo-like kinase 1 (PLK-1) (Genbank Accession No. NM_005030; Barr et al., Nat. Rev. Mol. Cell. Biol., 5:429-440 (2004)); tyrosine kinases such as WEE1 (Genbank Accession Nos. NM_003390 and NM-001143976); inhibitors of apoptosis such as XIAP (Genbank Accession No. NM_001167); COP9 signalosome subunits such as CSN1, CSN2, CSN3, CSN4, CSN5 (JAB1; Genbank Accession No. NM_006837); CSN6, CSN7A, CSN7B, and CSN8; ubiquitin ligases such as COP1 (RFWD2; Genbank Accession Nos. NM_022457 and NM_001001740); and histone deacetylases such as HDAC1, HDAC2 (Genbank Accession No. NM_001527), HDAC3, HDAC4, HDAC5, HDAC6, HDAC7, HDAC8, HDAC9, etc. Non-limiting examples of siRNA molecules targeting the Eg5 and XIAP genes include those described in U.S. patent application Ser. No. 11/807,872, filed May 29, 2007, the disclosure of which is herein incorporated by reference in its entirety for all purposes. Non-limiting examples of siRNA molecules targeting the PLK-1 gene include those described in U.S. Patent Publication Nos. 20050107316 and 20070265438; and U.S. patent application Ser. No. 12/343,342, filed Dec. 23, 2008, the disclosures of which are herein incorporated by reference in their entirety for all purposes. Non-limiting examples of siRNA molecules targeting the CSN5 gene include those described in U.S. Provisional Application No. 61/045,251, filed Apr. 15, 2008, the disclosure of which is herein incorporated by reference in its entirety for all purposes.

Additional examples of gene sequences associated with tumorigenesis and cell transformation include translocation sequences such as MLL fusion genes, BCR-ABL (Wilda et al., Oncogene, 21:5716 (2002); Scherr et al., Blood, 101:1566 (2003)), TEL-AML1, EWS-FLI1, TLS-FUS, PAX3-FKHR, BCL-2, AML1-ETO, and AML1-MTG8 (Heidenreich et al., Blood, 101:3157 (2003)); overexpressed sequences such as multidrug resistance genes (Nieth et al., FEBS Lett., 545:144 (2003); Wu et al, Cancer Res. 63:1515 (2003)), cyclins (Li et al., Cancer Res., 63:3593 (2003); Zou et al., Genes Dev., 16:2923 (2002)), beta-catenin (Verma et al., Clin Cancer Res., 9:1291 (2003)), telomerase genes (Kosciolek et al., Mol Cancer Ther., 2:209 (2003)), c-MYC, N-MYC, BCL-2, growth factor receptors (e.g., EGFR/ErbB1 (Genbank Accession Nos. NM_005228, NM_201282, NM_201283, and NM_201284; see also, Nagy et al. Exp. Cell Res., 285:39-49 (2003), ErbB2/HER-2 (Genbank Accession Nos. NM_004448 and NM_001005862), ErbB3 (Genbank Accession Nos. NM_001982 and NM_001005915), and ErbB4 (Genbank Accession Nos. NM_005235 and NM_001042599); and mutated sequences such as RAS (reviewed in Tuschl and Borkhardt, Mol. Interventions, 2:158 (2002)). Non-limiting examples of siRNA molecules targeting the EGFR gene include those described in U.S. patent application Ser. No. 11/807,872, filed May 29, 2007, the disclosure of which is herein incorporated by reference in its entirety for all purposes.

Silencing of sequences that encode DNA repair enzymes find use in combination with the administration of chemotherapeutic agents (Collis et al., Cancer Res., 63:1550 (2003)). Genes encoding proteins associated with tumor migration are also target sequences of interest, for example, integrins, selectins, and metalloproteinases. The foregoing examples are not exclusive. Those of skill in the art will understand that any whole or partial gene sequence that facilitates or promotes tumorigenesis or cell transformation, tumor growth, or tumor migration can be included as a template sequence.

Angiogenic genes are able to promote the formation of new vessels. Of particular interest is vascular endothelial growth factor (VEGF) (Reich et al., Mol. Vis., 9:210 (2003)) or VEGFR. siRNA sequences that target VEGFR are set forth in, e.g., GB 2396864; U.S. Patent Publication No. 20040142895; and CA 2456444, the disclosures of which are herein incorporated by reference in their entirety for all purposes.

Anti-angiogenic genes are able to inhibit neovascularization. These genes are particularly useful for treating those cancers in which angiogenesis plays a role in the pathological development of the disease. Examples of anti-angiogenic genes include, but are not limited to, endostatin (see, e.g., U.S. Pat. No. 6,174,861), angiostatin (see, e.g., U.S. Pat. No. 5,639,725), and VEGFR2 (see, e.g., Decaussin et al., J. Pathol., 188: 369-377 (1999)), the disclosures of which are herein incorporated by reference in their entirety for all purposes. Immunomodulator genes are genes that modulate one or more immune responses. Examples of immunomodulator genes include, without limitation, cytokines such as growth factors (e.g., TGF-α, TGF-β, EGF, FGF, IGF, NGF, PDGF, CGF, GM-CSF, SCF, etc.), interleukins (e.g., IL-2, IL-4, IL-12 (Hill et al., J. Immunol., 171:691 (2003)), IL-15, IL-18, IL-20, etc.), interferons (e.g., IFN-α, IFN-β, IFN-γ, etc.) and TNF. Fas and Fas ligand genes are also immunomodulator target sequences of interest (Song et al., Nat. Med., 9:347 (2003)). Genes encoding secondary signaling molecules in hematopoietic and lymphoid cells are also included in the present invention, for example, Tec family kinases such as Bruton's tyrosine kinase (Btk) (Heinonen et al., FEBS Lett., 527:274 (2002)).

Cell receptor ligands include ligands that are able to bind to cell surface receptors (e.g., insulin receptor, EPO receptor, G-protein coupled receptors, receptors with tyrosine kinase activity, cytokine receptors, growth factor receptors, etc.), to modulate (e.g., inhibit, activate, etc.) the physiological pathway that the receptor is involved in (e.g., glucose level modulation, blood cell development, mitogenesis, etc.). Examples of cell receptor ligands include, but are not limited to, cytokines, growth factors, interleukins, interferons, erythropoietin (EPO), insulin, glucagon, G-protein coupled receptor ligands, etc. Templates coding for an expansion of trinucleotide repeats (e.g., CAG repeats) find use in silencing pathogenic sequences in neurodegenerative disorders caused by the expansion of trinucleotide repeats, such as spinobulbular muscular atrophy and Huntington's Disease (Caplen et al., Hum. Mol. Genet., 11:175 (2002)).

Certain other target genes, which may be targeted by a nucleic acid (e.g., by siRNA) to downregulate or silence the expression of the gene, include but are not limited to, Actin, Alpha 2, Smooth Muscle, Aorta (ACTA2), Alcohol dehydrogenase 1A (ADH1A), Alcohol dehydrogenase 4 (ADH4), Alcohol dehydrogenase 6 (ADH6), Afamin (AFM), Angiotensinogen (AGT), Serine-pyruvate aminotransferase (AGXT), Alpha-2-HS-glycoprotein (AHSG), Aldo-keto reductase family 1 member C4 (AKR1C4), Serum albumin (ALB), alpha-1-microglobulin/bikunin precursor (AMBP), Angiopoietin-related protein 3 (ANGPTL3), Serum amyloid P-component (APCS), Apolipoprotein A-II (APOA2), Apolipoprotein B-100 (APOB), Apolipoprotein C3 (APOC3), Apolipoprotein C-IV (APOC4), Apolipoprotein F (APOF), Beta-2-glycoprotein 1 (APOH), Aquaporin-9 (AQP9), Bile acid-CoA:amino acid N-acyltransferase (BAAT), C4b-binding protein beta chain (C4BPB), Putative uncharacterized protein encoded by LINC01554 (C5orf27), Complement factor 3 (C3), Complement Factor 5 (C5), Complement component C6 (C6), Complement component C8 alpha chain (C8A), Complement component C8 beta chain (C8B), Complement component C8 gamma chain (C8G), Complement component C9 (C9), Calmodulin Binding Transcription Activator 1 (CAMTA1), CD38 (CD38), Complement Factor B (CFB), Complement factor H-related protein 1 (CFHR1), Complement factor H-related protein 2 (CFHR2), Complement factor H-related protein 3 (CFHR3), Cannabinoid receptor 1 (CNR1), ceruloplasmin (CP), carboxypeptidase B2 (CPB2), Connective tissue growth factor (CTGF), C-X-C motif chemokine 2 (CXCL2), Cytochrome P450 1A2 (CYP1A2), Cytochrome P450 2A6 (CYP2A6), Cytochrome P450 2C8 (CYP2C8), Cytochrome P450 2C9 (CYP2C9), Cytochrome P450 Family 2 Subfamily D Member 6 (CYP2D6), Cytochrome P450 2E1 (CYP2E1), Phylloquinone omega-hydroxylase CYP4F2 (CYP4F2), 7-alpha-hydroxycholest-4-en-3-one 12-alpha-hydroxylase (CYP8B1), Dipeptidyl peptidase 4 (DPP4), coagulation factor 12 (F12), coagulation factor II (thrombin) (F2), coagulation factor IX (F9), fibrinogen alpha chain (FGA), fibrinogen beta chain (FGB), fibrinogen gamma chain (FGG), fibrinogen-like 1 (FGL1), flavin containing monooxygenase 3 (FMO3), flavin containing monooxygenase 5 (FMO5), group-specific component (vitamin D binding protein) (GC), Growth hormone receptor (GHR), glycine N-methyltransferase (GNMT), hyaluronan binding protein 2 (HABP2), hepcidin antimicrobial peptide (HAMP), hydroxyacid oxidase (glycolate oxidase) 1 (HAO1), HGF activator (HGFAC), haptoglobin-related protein; haptoglobin (HPR), hemopexin (HPX), histidine-rich glycoprotein (HRG), hydroxysteroid (11-beta) dehydrogenase 1 (HSD 11B1), hydroxysteroid (17-beta) dehydrogenase 13 (HSD17B13), Inter-alpha-trypsin inhibitor heavy chain H1 (ITIH1), Inter-alpha-trypsin inhibitor heavy chain H2 (ITIH2), Inter-alpha-trypsin inhibitor heavy chain H3 (ITIH3), Inter-alpha-trypsin inhibitor heavy chain H4 (ITIH4), Prekallikrein (KLKB1), Lactate dehydrogenase A (LDHA), liver expressed antimicrobial peptide 2 (LEAP2), leukocyte cell-derived chemotaxin 2 (LECT2), Lipoprotein (a) (LPA), mannan-binding lectin serine peptidase 2 (MASP2), S-adenosylmethionine synthase isoform type-1 (MAT1A), NADPH Oxidase 4 (NOX4), Poly [ADP-ribose] polymerase 1 (PARP1), paraoxonase 1 (PON1), paraoxonase 3 (PON3), Vitamin K-dependent protein C (PROC), Retinol dehydrogenase 16 (RDH16), serum amyloid A4, constitutive (SAA4), serine dehydratase (SDS), Serpin Family A Member 1 (SERPINA1), Serpin A11 (SERPINA11), Kallistatin (SERPINA4), Corticosteroid-binding globulin (SERPINA6), Antithrombin-III (SERPINC1), Heparin cofactor 2 (SERPIND1), Serpin Family H Member 1 (SERPINH1), Solute Carrier Family 5 Member 2 (SLC5A2), Sodium/bile acid cotransporter (SLC10A1), Solute carrier family 13 member 5 (SLC13A5), Solute carrier family 22 member 1 (SLC22A1), Solute carrier family 25 member 47 (SLC25A47), Solute carrier family 2, facilitated glucose transporter member 2 (SLC2A2), Sodium-coupled neutral amino acid transporter 4 (SLC38A4), Solute carrier organic anion transporter family member 1B1 (SLCO1B1), Sphingomyelin Phosphodiesterase 1 (SMPD1), Bile salt sulfotransferase (SULT2A1), tyrosine aminotransferase (TAT), tryptophan 2,3-dioxygenase (TDO2), UDP glucuronosyltransferase 2 family, polypeptide B10 (UGT2B10), UDP glucuronosyltransferase 2 family, polypeptide B15 (UGT2B15), UDP glucuronosyltransferase 2 family, polypeptide B4 (UGT2B4) and vitronectin (VTN).

In addition to its utility in silencing the expression of any of the above-described genes for therapeutic purposes, certain nucleic acids (e.g., siRNA) described herein are also useful in research and development applications as well as diagnostic, prophylactic, prognostic, clinical, and other healthcare applications. As a non-limiting example, certain nucleic acids (e.g., siRNA) can be used in target validation studies directed at testing whether a gene of interest has the potential to be a therapeutic target. Certain nucleic acids (e.g., siRNA) can also be used in target identification studies aimed at discovering genes as potential therapeutic targets.

Generating siRNA Molecules

siRNA can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. In some embodiments, siRNA may be produced enzymatically or by partial/total organic synthesis, and modified ribonucleotides can be introduced by in vitro enzymatic or organic synthesis. In certain instances, each strand is prepared chemically. Methods of synthesizing RNA molecules are known in the art, e.g., the chemical synthesis methods as described in Verma and Eckstein (1998) or as described herein.

Methods for isolating RNA, synthesizing RNA, hybridizing nucleic acids, making and screening cDNA libraries, and performing PCR are well known in the art (see, e.g., Gubler and Hoffian, Gene, 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994). The disclosures of these references are herein incorporated by reference in their entirety for all purposes.

Typically, siRNA are chemically synthesized. The oligonucleotides that comprise the siRNA molecules of the invention can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of oligonucleotides makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol. Alternatively, syntheses at the 0.2 μmol scale can be performed on a 96-well plate synthesizer from Protogene (Palo Alto, Calif.). However, a larger or smaller scale of synthesis is also within the scope of this invention. Suitable reagents for oligonucleotide synthesis, methods for RNA deprotection, and methods for RNA purification are known to those of skill in the art.

siRNA molecules can be assembled from two distinct oligonucleotides, wherein one oligonucleotide comprises the sense strand and the other comprises the antisense strand of the siRNA. For example, each strand can be synthesized separately and joined together by hybridization or ligation following synthesis and/or deprotection.

### Linking Group

The conjugates of the invention may include one or more linking groups (e.g. L³ or L⁴). The structure of each linking group can vary, provided the conjugate functions as described herein. For example, the structure of each linking group vary in length and atom composition, and each linking group can be branched, non-branched, cyclic, or a combination thereof. The linking group may also modulate the solubility, stability, or aggregation properties of the conjugate.

In one embodiment each linking group comprises about 3-1000 atoms. In one embodiment each linking group comprises about 3-500 atoms. In one embodiment each linking group comprises about 3-200 atoms. In one embodiment each linking group comprises about 3-50 atoms. In one embodiment each linking group comprises about 10-1000 atoms.

In one embodiment each linking group comprises about 10-500 atoms. In one embodiment each linking group comprises about 10-200 atoms. In one embodiment each linking group comprises about 10-50 atoms.

In one embodiment each linking group comprises atoms selected from H, C, N, S and O.

In one embodiment each linking group comprises atoms selected from H, C, N, S, P and O.

In one embodiment each linking group comprises a branched or unbranched, saturated or unsaturated, hydrocarbon chain, having from about 1 to 1000 (or 1-750, 1-500, 1-250, 1-100, 1-50, 1-25, 1-10, 1-5, 5-1000, 5-750, 5-500, 5-250, 5-100, 5-50, 5-25, 5-10 or 2-5 carbon atoms) wherein one or more of the carbon atoms is optionally replaced independently by —O—, —S, —N(R^{a})—, 3-7 membered heterocycle, 5-6-membered heteroaryl or carbocycle and wherein each chain, 3-7 membered heterocycle, 5-6-membered heteroaryl or carbocycle is optionally and independently substituted with one or more (e.g. 1, 2, 3, 4, 5 or more) substituents selected from (C₁-C₆)alkyl, (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, —N(R^{a})₂, hydroxy, oxo (═O), carboxy, aryl, aryloxy, heteroaryl, and heteroaryloxy, wherein each R is independently H or (C₁-C₆)alkyl. In one embodiment the linker comprises a branched or unbranched, saturated or unsaturated, hydrocarbon chain, having from about 1 to 1000 (or 1-750, 1-500, 1-250, 1-100, 1-50, 1-25, 1-10, 1-5, 5-1000, 5-750, 5-500, 5-250, 5-100, 5-50, 5-25, 5-10 or 2-5 carbon atoms) wherein one or more of the carbon atoms is optionally replaced independently by —O—, —S, —N(R^{a})—, wherein each R^{a} is independently H or (C₁-C₆)alkyl.

In one embodiment each linking group comprises a polyethylene glycol. In one embodiment the linking group comprises a polyethylene glycol linked to the remainder of the targeted conjugate by a carbonyl group. In one embodiment the polyethylene glycol comprises about 1 to about 500 or about 5 to about 500 or about 3 to about 100 repeat (e.g., —CH₂CH₂O—) units (Greenwald, R. B., et al., Poly (ethylene glycol) Prodrugs: Altered Pharmacokinetics and Pharmacodynamics, Chapter, 2.3.1., 283-338; Filpula, D., et al., Releasable PEGylation of proteins with customized linkers, Advanced Drug Delivery, 60, 2008, 29-49; Zhao, H., et al., Drug Conjugates with Poly(Ethylene Glycol), Drug Delivery in Oncology, 2012, 627-656).

### EMBODIMENTS

In one embodiment, A is a targeting ligand that specifically binds to a molecule on the surface of the target cell.

In one embodiment, the nucleic acid conjugate and the membrane-destabilizing polymer are administered separately.

In one embodiment, the membrane-destabilizing polymer is administered after administration of the nucleic acid conjugate.

In one embodiment, the nucleic acid conjugate and the membrane-destabilizing polymer are administered together within a single composition.

In one embodiment, the targeting ligand and T⁵ are different and either (i) specifically bind to the same cell surface molecule or (ii) specifically bind to a different cell surface molecule on the target cell.

In one embodiment, the targeting ligand and the T⁵ are the same and each specifically binds to the same cell surface molecule.

In one embodiment, the cell is a secretory cell, a chondrocyte, an epithelial cell, a nerve cell, a muscle cell, a blood cell, an endothelial cell, a pericyte, a fibroblast, a glial cell, or a dendritic cell.

In one embodiment, the cell is a cancer cell, an immune cell, a bacterially-infected cell, a virally-infected cell, or a cell having an abnormal metabolic activity.

In one embodiment, the targeting ligand specifically binds to a cell surface molecule selected from the group consisting of transferrin receptor type 1, transferrin receptor type 2, the EGF receptor, HER2/Neu, a VEGF receptor, a PDGF receptor, an integrin, an NGF receptor, CD2, CD3, CD4, CD8, CD19, CD20, CD22, CD33, CD43, CD38, CD56, CD69, the asialoglycoprotein receptor (ASGPR), prostate-specific membrane antigen (PSMA), a folate receptor, and a sigma receptor.

In one embodiment, the targeting ligand comprises a small molecule targeting moiety.

In one embodiment, the small molecule targeting moiety is a sugar, a vitamin, a bisphosphonate, or an analogue thereof.

In one embodiment, the sugar is selected from lactose, galactose. N-acetyl galactosamine (NAG), mannose, and mannose-6-phosphate (M6P).

In one embodiment, the vitamin is folate.

In one embodiment, the targeting ligand comprises a protein.

In one embodiment, the protein is an antibody, a peptide aptamer, or a protein derived from a natural ligand of the cell surface molecule.

In one embodiment, the targeting ligand comprises a peptide.

In one embodiment, the peptide is an integrin-binding peptide, a LOX-1-binding peptide, and epidermal growth factor (EGF) peptide, a neurotensin peptide, an NL4 peptide, or a YIGSR laminin peptide.

In one embodiment, the cell is a hepatocyte.

In one embodiment, the targeting ligand specifically binds to the asialoglycoprotein receptor (ASGPR).

In one embodiment, the targeting ligand comprises an N-acetylgalactosamine (NAG) residue.

In one embodiment, the membrane destabilizing polymer comprises of three regions:

a monosaccharide,

a hydrophilic region comprising polyethyleneglycol methacrylate 4-5 (PEGMA 4-5) and hydroxyethyl methacrylate (HMA); and

a region that provides endosomal release

In one embodiment, the membrane destabilizing polymer is a polymer of formula (XX)

T⁵-L-[PEGMAₘ-M²ₙ]ᵥ-[DMAEMA_{q}-PAAᵣ-BMAₛ]_{w} (XX)

wherein:

PEGMA is polyethyleneglycol methacrylate residue with 2-20 ethylene glycol units;

M² is a methacrylate residue selected from the group consisting of
a (C₄-C₁₈)alkyl-methacrylate residue;
a (C₄-C₁₈) branched alkyl-methacrylate residue;
a cholesteryl methacrylate residue;
a (C₄-C₁₈)alkyl-methacrylate residue substituted with one or more fluorine atoms; and
a (C₄-C₁₈) branched alkyl-methacrylate residue substituted with one or more fluorine atoms;

BMA is butyl methacrylate residue;

PAA is propyl acrylic acid residue;

DMAEMA is dimethylaminoethyl methacrylate residue;

m and n are each a mole fraction greater than 0, wherein m is greater than n and m+n=1;

q is a mole fraction of 0.2 to 0.75;

r is a mole fraction of 0.05 to 0.6;

s is a mole fraction of 0.2 to 0.75;

q+r+s=1;

v is 1 to 25 kDa;

w is 1 to 25 kDa;

T⁵ is a targeting moiety (e.g., a peptide, polymer or saccharide); and

L is absent or is a linking moiety.

In one embodiment, M² is selected from the group consisting of:
2,2,3,3,4,4,4-heptafluorobutyl methacrylate residue,
3,3,4,4,5,6,6,6-octafluoro-5(trifluoromethyl)hexyl methacrylate residue,
2,2,3,3,4,4,5,5,6,6,7,7,8,8,8-pentadecafluorooctyl 2-methylacrylate residue,
3,3,4,4,5,5,6,6,6-nonafluorohexyl methacrylate residue,
3,3,4,4,5,5,6,6,7,7,8,8,8-tridecafluorooctyl methacrylate residue,
1,1,1-trifluoro-2-(trifluoromethyl)-2-hydroxy-4-methyl-5-pentyl methacrylate residue, 2-[(1′,1′, 1′-trifluoro-2′-(trifluoro methyl)-2′-hydroxy)propyl]-3-norbornyl methacrylate residue, 2-ethylhexyl methacrylate residue,
butyl methacrylate residue,
hexyl methacrylate residue,
octyl methacrylate residue,
n-decyl methacrylate residue,
lauryl methacrylate residue,
myristyl methacrylate residue,
stearyl methacrylate residue,
cholesteryl methacrylate residue,
ethylene glycol phenyl ether methacrylate residue,
2-propenoic acid, 2-methyl-, 2-phenylethyl ester residue,
2-propenoic acid, 2-methyl-, 2-[[(1,1-dimethylethoxy)carbonyl]amino]ethyl ester residue,
2-propenoic acid, 2-methyl-, 2-(1H-imidazol-1-yl)ethyl ester residue,
2-propenoic acid, 2-methyl-, cyclohexyl ester residue,
2-propenoic acid, 2-methyl-, 2-[bis(1-methylethyl)amino]ethyl ester residue,
2-propenoic acid, 2-methyl-, 3-methylbutyl ester residue, neopentyl methacrylate residue,
tert-butyl methacrylate residue,
3,3,5-trimethyl cyclohexyl methacrylate residue,
2-hydroxypropyl methacrylate residue,
5-nonyl methacrylate residue,
2-butyl-1-octyl methacrylate residue,
2-hexyl-1-decyl methacrylate residue, and
2-(tert-butyl amino)ethyl methacrylate residue.

In one embodiment, PEGMA has 4-5 ethylene glycol units or 7-8 ethylene glycol units.

In one embodiment, T¹ and L are present and T¹ comprises an N-acetylgalactosamine (NAG) residue.

In one embodiment, L is comprises a polyethylene glycol (PEG) moiety having 2-20 ethylene glycol units.

In one embodiment, the membrane destabilizing polymer is a polymer of formula (XXI):

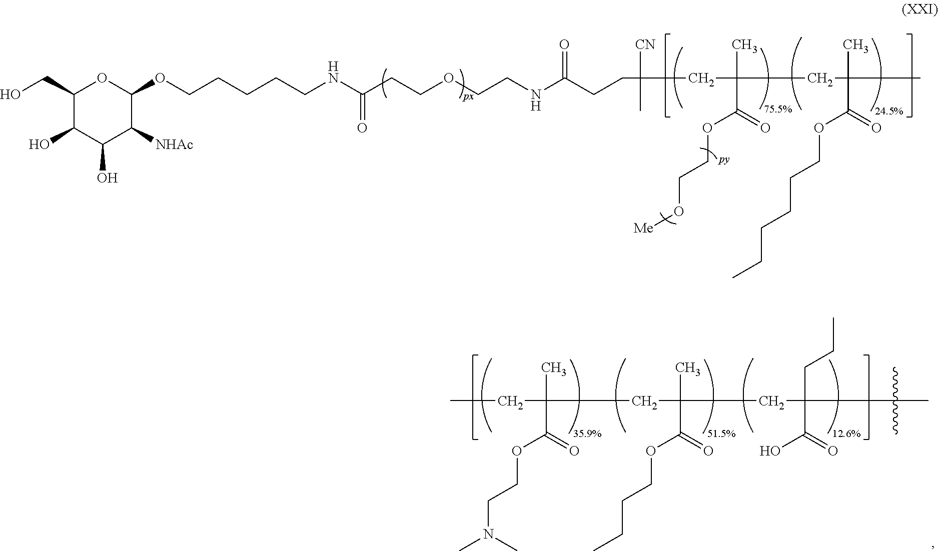

wherein px is an integer of from about 2 to about 50, e.g., from about 2 to about 20, e.g., from 4 to 12 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50). In some embodiments, px is an integer of from about 8 to about 16 (e.g., 8, 9, 10, 11, 12, 13, 14, 15, or 16). In some embodiments, px is about 12. In some embodiments, py is an integer of from about 2 to about 20 (e.g., 2, 3, 4, 5, 6, 7, 8, 9, 10, 11, 12, 13, 14, 15, 16, 17, 18, 19, or 20). In some embodiments, py is an integer of from about 2 to about 10 (e.g., 2, 3, 4, 5, 6, 7, 8, 9, or 10). In some embodiments, py is an integer of from about 4 to about 5 (e.g., 4 or 5).

In one embodiment, the compound of formula (X) is a compound of formula (I):

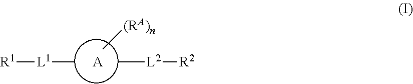

wherein:

R¹ a is targeting ligand;

L¹ is absent or a linking group;

L² is absent or a linking group;

R² is the nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10;

or a salt thereof.

In one embodiment,

R¹ a is targeting ligand;

L¹ is absent or a linking group;

L² is absent or a linking group;

R² is the nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B} and C₁₋₈ alkyl that is optionally substituted with one or more groups independently selected from halo, hydroxy, and C₁₋₃ alkoxy; R^{B} is hydrogen, a protecting group, a covalent bond to a solid support, or a bond to a linking group that is bound to a solid support; and

n is 0, 1, 2, 3, 4, 5, 6, 7, 8, 9, or 10.

In one embodiment, R¹ is —C(H)₍₃₋ₚ₎(L³-saccharide)ₚ,

wherein each L³ is independently a linking group;

p is 1, 2, or 3; and

saccharide is a monosaccharide or disaccharide.

In one embodiment, the saccharide is:

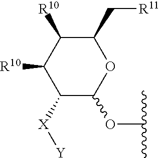

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy

In one embodiment, the saccharide is selected from the group consisting of:

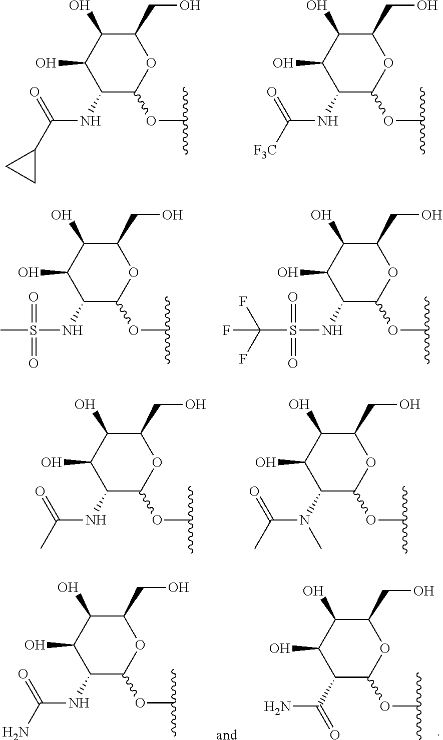

In one embodiment, the saccharide is:

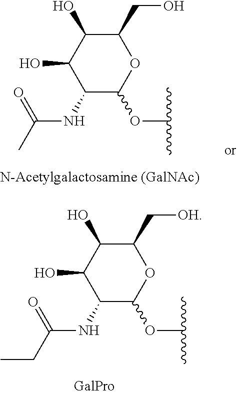 [SMILES: *OC1O[C@H](CO)[C@H](O)[C@H](O)[C@H]1NC(C)=O ; "or" ; *OC1O[C@H](CO)[C@H](O)[C@H](O)[C@H]1NC(=O)CC]

In one embodiment, each L³ is independently a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment, each L³ is independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment, L³ is:

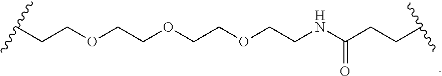 [SMILES: *CCOCCOCCOCCNC(=O)CC*]

In one embodiment, R¹ is:

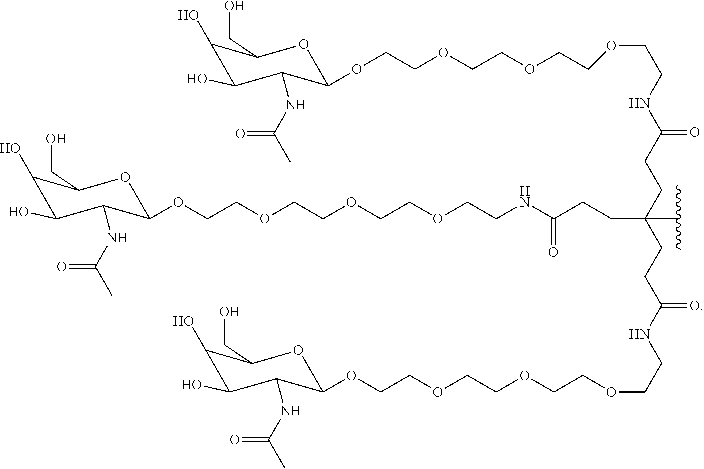 [SMILES: *C(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In one embodiment, R¹ is:

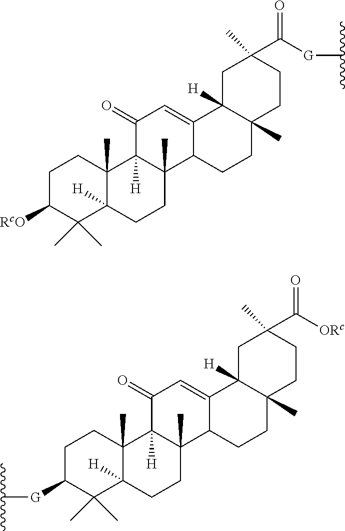

wherein:

G is —NH— or —O—;

R^{C} is hydrogen, (C₁-C₈)alkyl, (C₁-C₈)haloalkyl, (C₁-C₈)alkoxy, (C₁-C₆)alkanoyl, (C₃-C₂₀)cycloalkyl, (C₃-C₂₀)heterocycle, aryl, heteroaryl, monosaccharide, disaccharide or trisaccharide; and wherein the cycloalkyl, heterocyle, ary, heteroaryl and saccharide are optionally substituted with one or more groups independently selected from the group consisting of halo, carboxyl, hydroxyl, amino, (C₁-C₄)alkyl, (C₁-C₄)haloalkyl, (C₁-C₄)alkoxy and (C₁-C₄)haloalkoxy.

In one embodiment, R^{C} is:

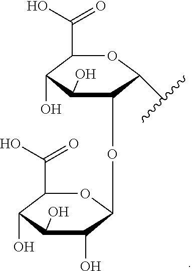

In one embodiment, R¹ is:

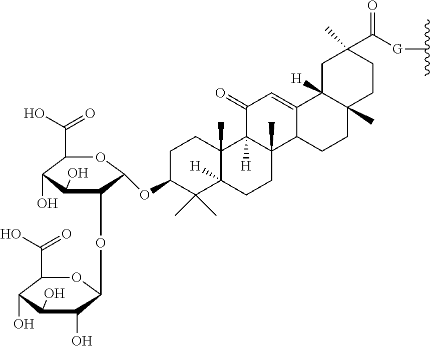

In one embodiment, R^{C} is:

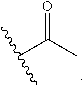 [SMILES: *C(C)=O]

In one embodiment, G is —NH—.

In one embodiment, R¹ is:

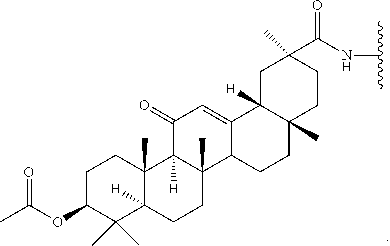

In one embodiment, R¹ is:

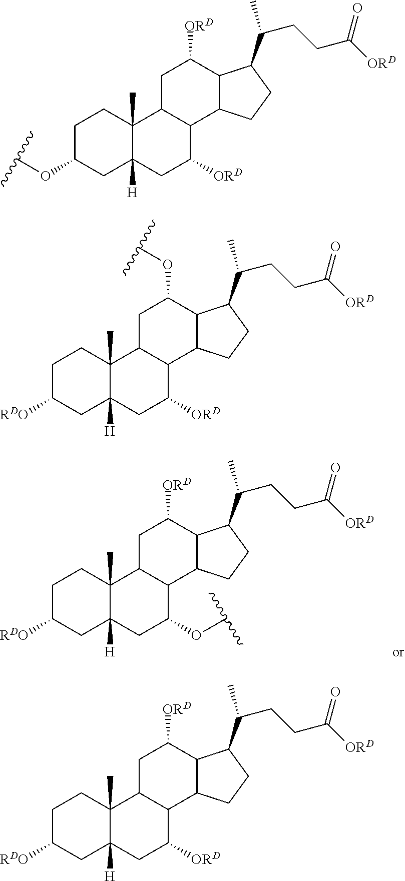

wherein each R^{D} is independently selected from the group consisting of hydrogen, (C₁-C₆)alkyl, (C₉-C₂₀)alkylsilyl, (R^{W})₃Si—, (C₂-C₆)alkenyl, tetrahydropyranyl, (C₁-C₆)alkanoyl, benzoyl, aryl(C₁-C₃)alkyl, TMTr (Trimethoxytrityl), DMTr (Dimethoxytrityl), MMTr (Monomethoxytrityl), and Tr (Trityl); and

each R^{W} is independently selected from the group consisting of (C₁-C₄)alkyl and aryl.

In one embodiment, L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment, L¹ and L² are independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment, L¹ and L² are independently, a divalent, branched or unbranched, saturated or unsaturated, hydrocarbon chain, having from 1 to 14 carbon atoms, wherein one or more (e.g. 1, 2, 3, or 4) of the carbon atoms in the hydrocarbon chain is optionally replaced —O—, —NR^{X}—, —NR^{X}—C(═O)—, —C(═O)—NR^{X}- or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment, L¹ is connected to R¹ through —NH—, —O—, —S—, —(C═O)—, —(C═O)—NH—, —NH—(C═O)—, —(C═O)—O—, —NH—(C═O)—NH—, or —NH—(SO₂)—.

In one embodiment, L² is connected to R² through —O—.

In one embodiment, L¹ is selected from the group consisting of:

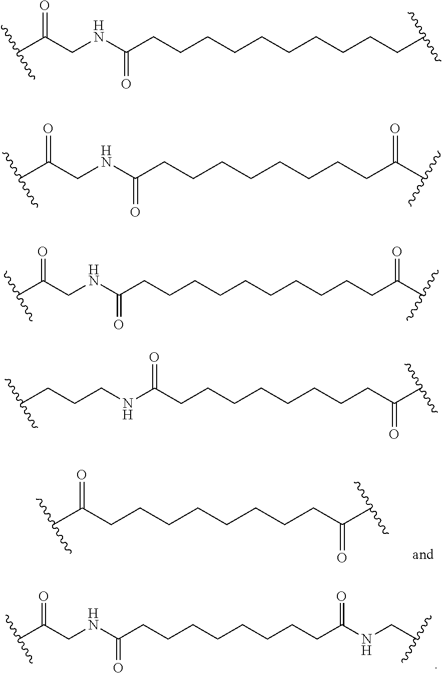

In one embodiment, L² is —CH₂—O- or —CH₂—CH₂—O—.

In one embodiment,

the compound of formula (I) is a compound of formula (Ia):

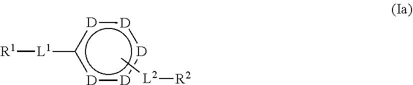

wherein:

each D is independently selected from the group consisting of

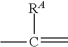

### and —N═;

or a salt thereof.

In one embodiment, the compound of formula (I) is selected from the group consisting of:

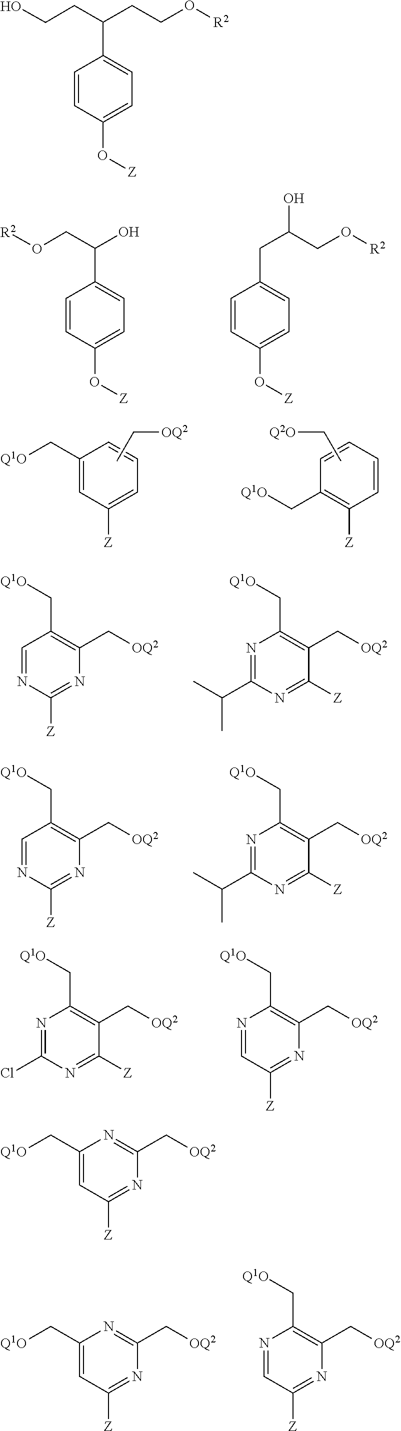

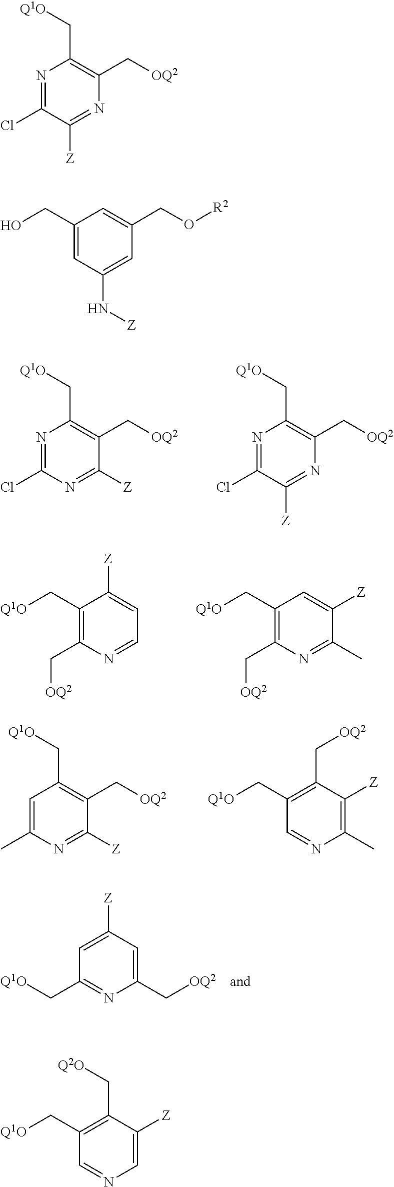

wherein:

Q¹ is hydrogen and Q² is R²; or Q¹ is R² and Q² is hydrogen; and

Z is -L¹-R¹.

In one embodiment, the compound of formula (I) is a compound of formula (Ib):

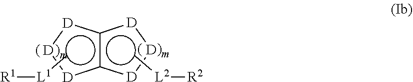

wherein:

each D is independently selected from the group consisting of

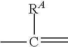

### and —N═; and

each m is independently 1 or 2.

In one embodiment, the compound of formula (I) is selected from the group consisting of:

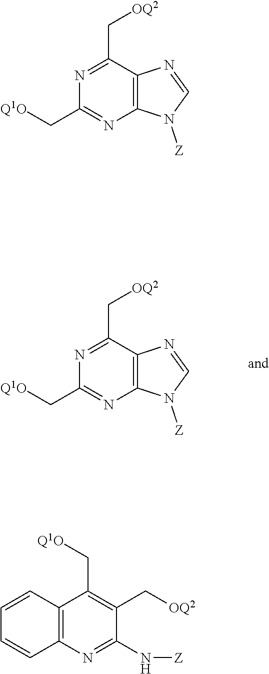

wherein:

Q¹ is hydrogen and Q² is R²; or Q is R² and Q² is hydrogen; and

Z is -L¹-R¹.

In one embodiment, the compound of formula (I) is a compound of formula (Ic):

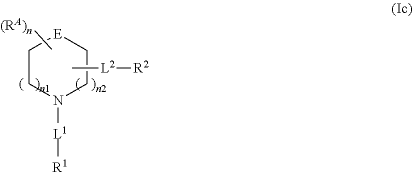

wherein:

E is —O— or —CH₂—;

n is selected from the group consisting of 0, 1, 2, 3, and 4; and

n1 and n2 are each independently selected from the group consisting of 0, 1, 2, and 3.

In one embodiment, the compound of formula (I) is selected from the group consisting of:

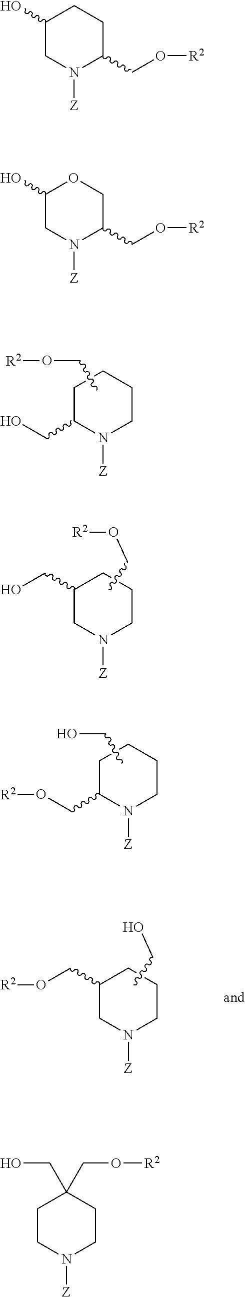

wherein: Z is -L¹-R¹.

In one embodiment, -A-L²-R² is:

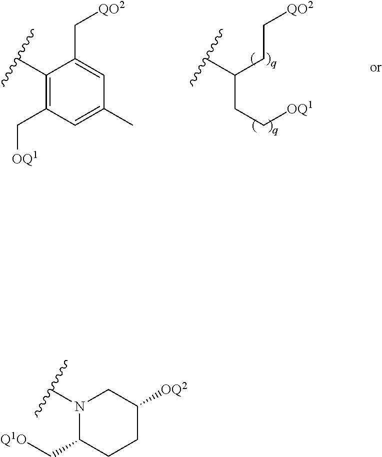

wherein:

Q¹ is hydrogen and Q² is R²; or Q¹ is R² and Q² is hydrogen; and

each q is independently 0, 1, 2, 3, 4 or 5.

In one embodiment, the compound of formula (I) is selected from the group consisting of:

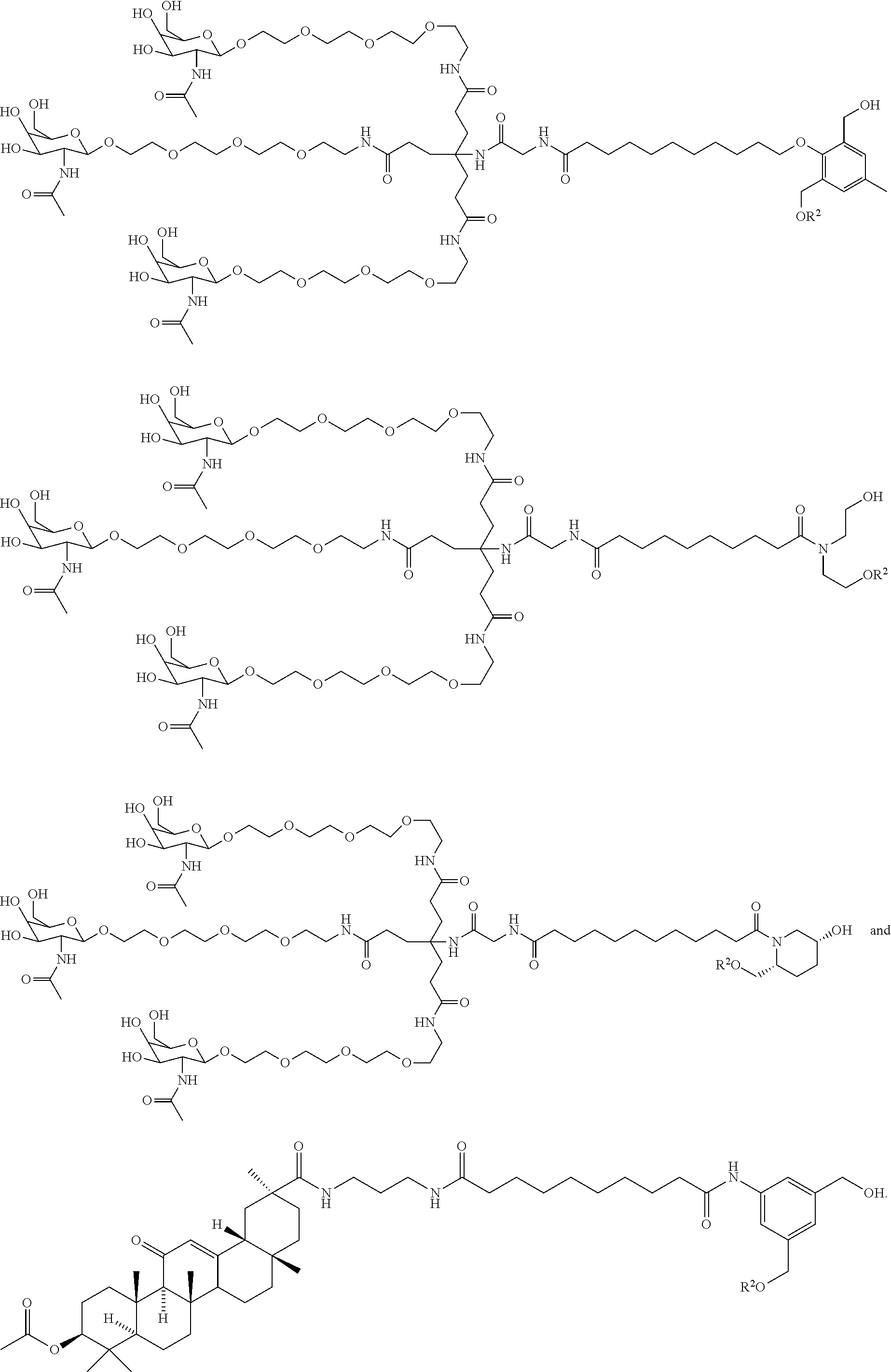

In one embodiment, R¹ is selected from the group consisting of

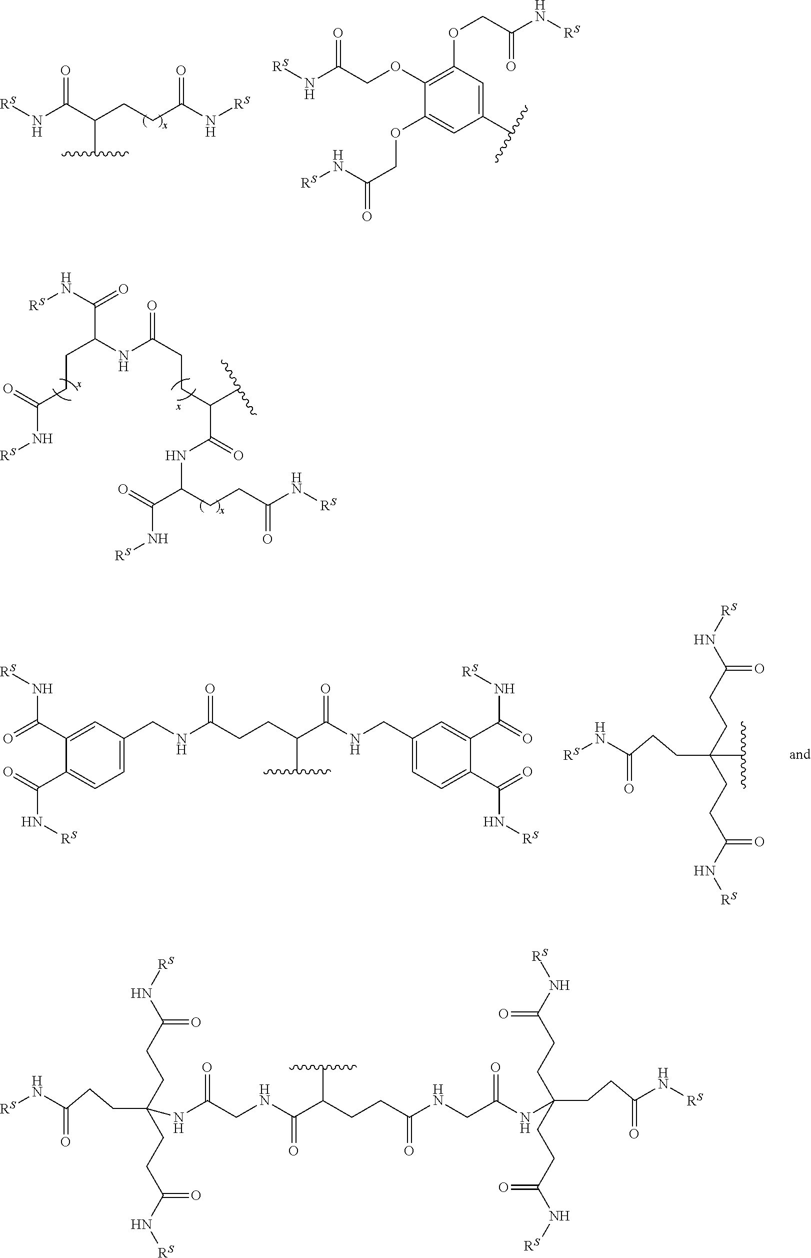

wherein:

R^{S} is

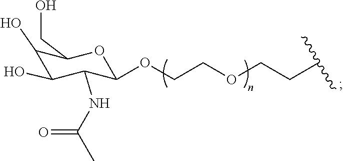

n is 2, 3, or 4; and

x is 1 or 2.

In one embodiment, L¹ is selected from the group consisting of:

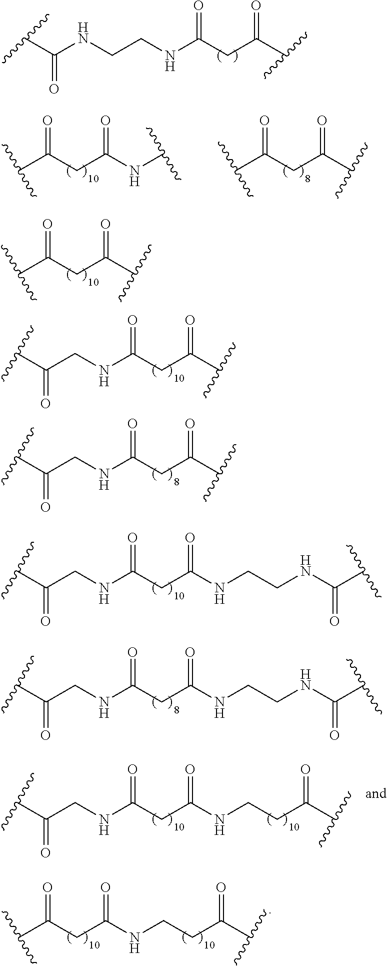

In one embodiment, A is absent, phenyl, pyrrolidinyl, or cyclopentyl.

In one embodiment, L² is C₁₋₄ alkylene-O— that is optionally substituted with hydroxy.

In one embodiment, L² is —CH₂O—, —CH₂CH₂O—, or —CH(OH)CH₂O—.

In one embodiment, each R^{A} is independently hydroxy or C₁₋₈ alkyl that is optionally substituted with hydroxyl.

In one embodiment, each R^{A} is independently selected from the group consisting of hydroxy, methyl and —CH₂OH.

In one embodiment, the compound of formula (I) is a compound formula (Ig):

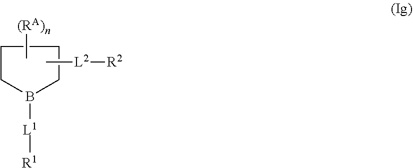

wherein:

B is —N— or —CH—;

L² is C₁₋₄ alkylene-O— that is optionally substituted with hydroxyl or halo; and

n is 0, 1, 2, 3, 4, 5, 6, or 7.

In one embodiment, the compound of formula (I) is selected from the group consisting of:

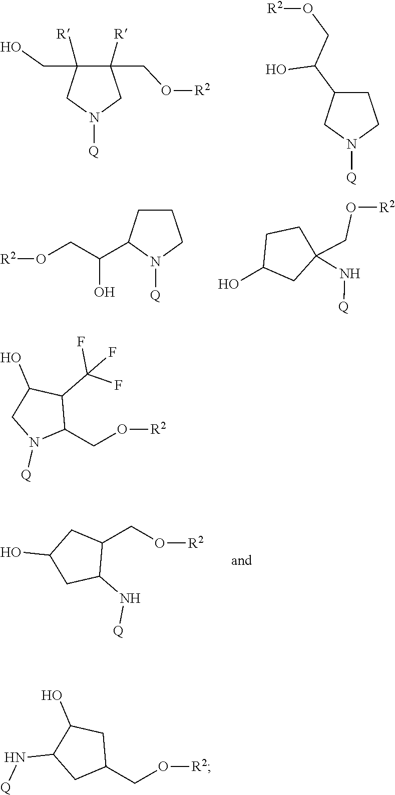

wherein Q is -L¹-R¹; and

R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

In one embodiment, the compound of formula (I) is selected from the group consisting of:

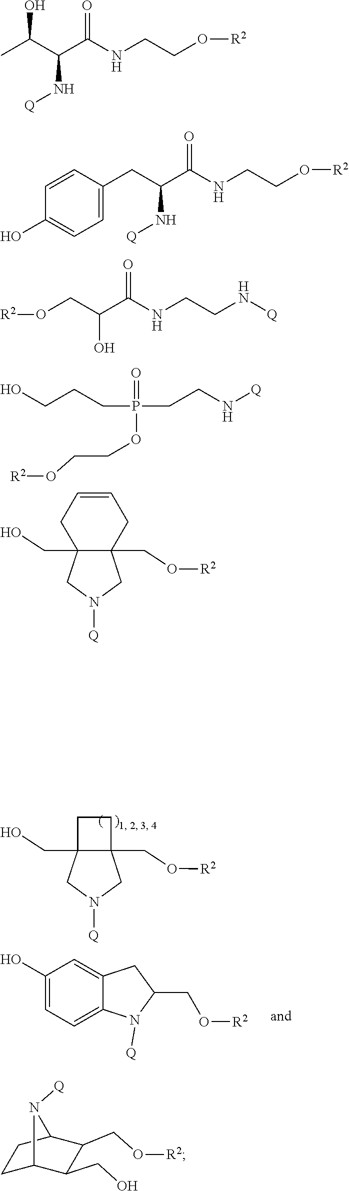

wherein Q is -L¹-R¹

In one embodiment, the compound of formula (I) is selected from the group consisting of:

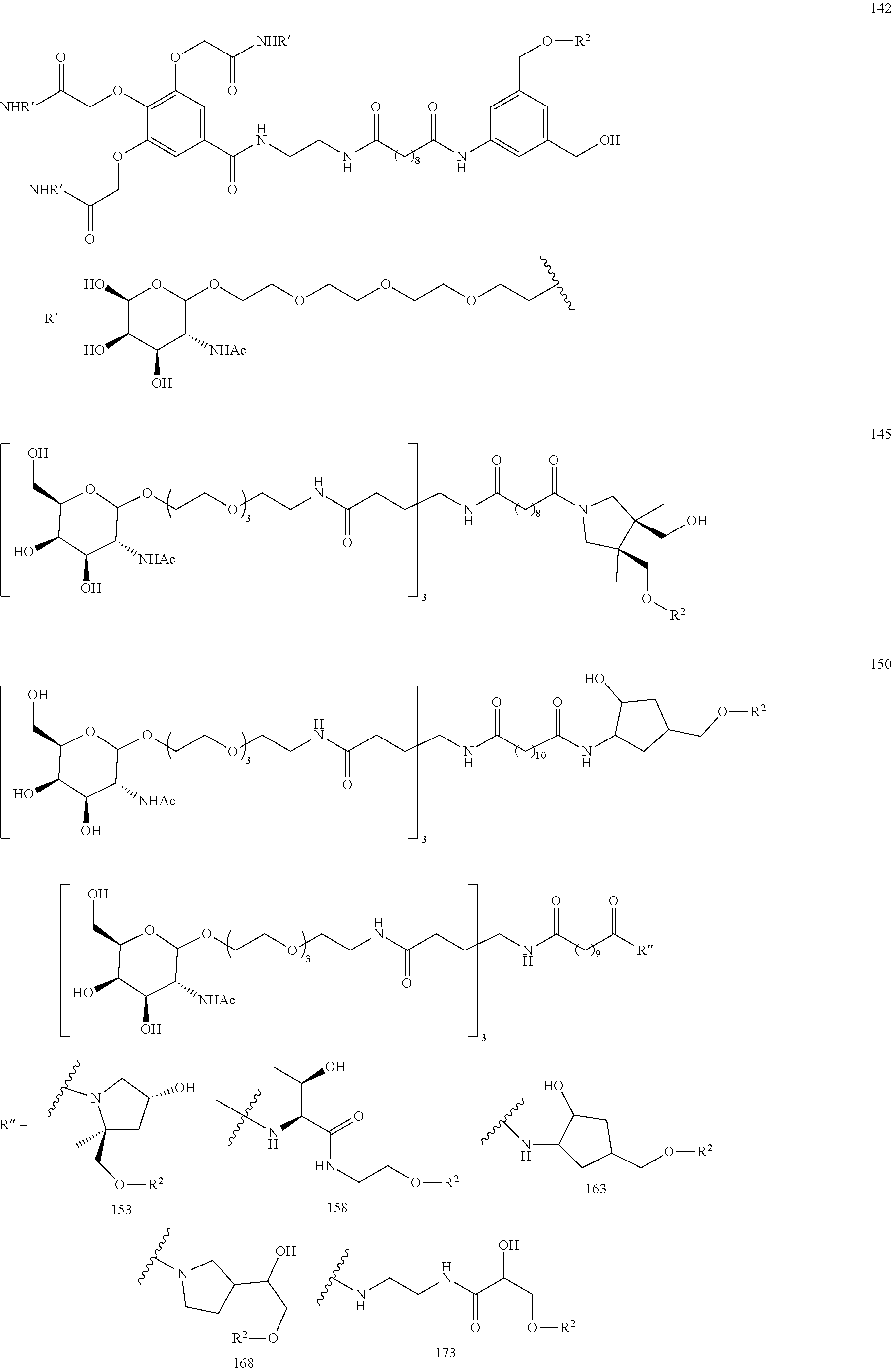

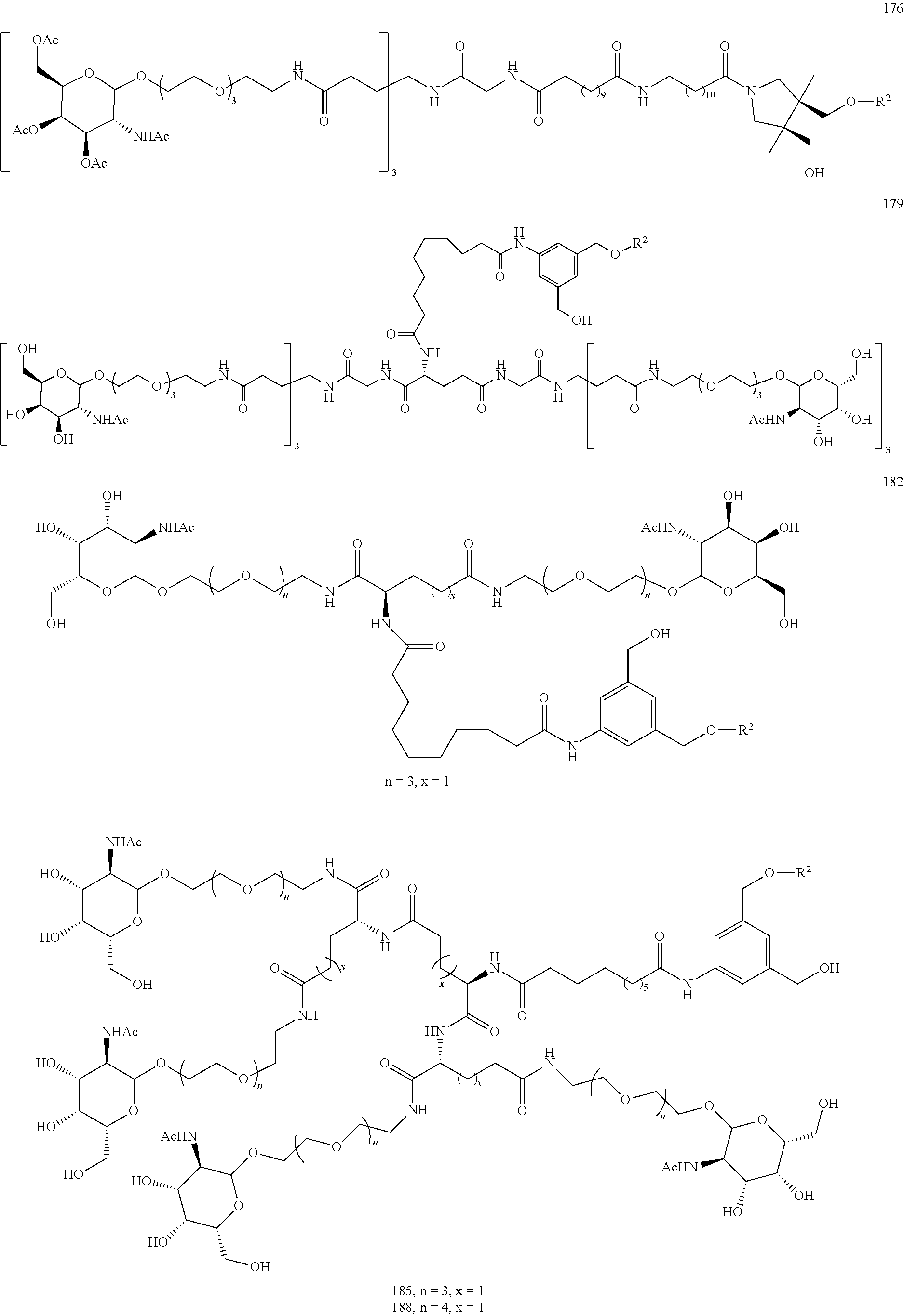

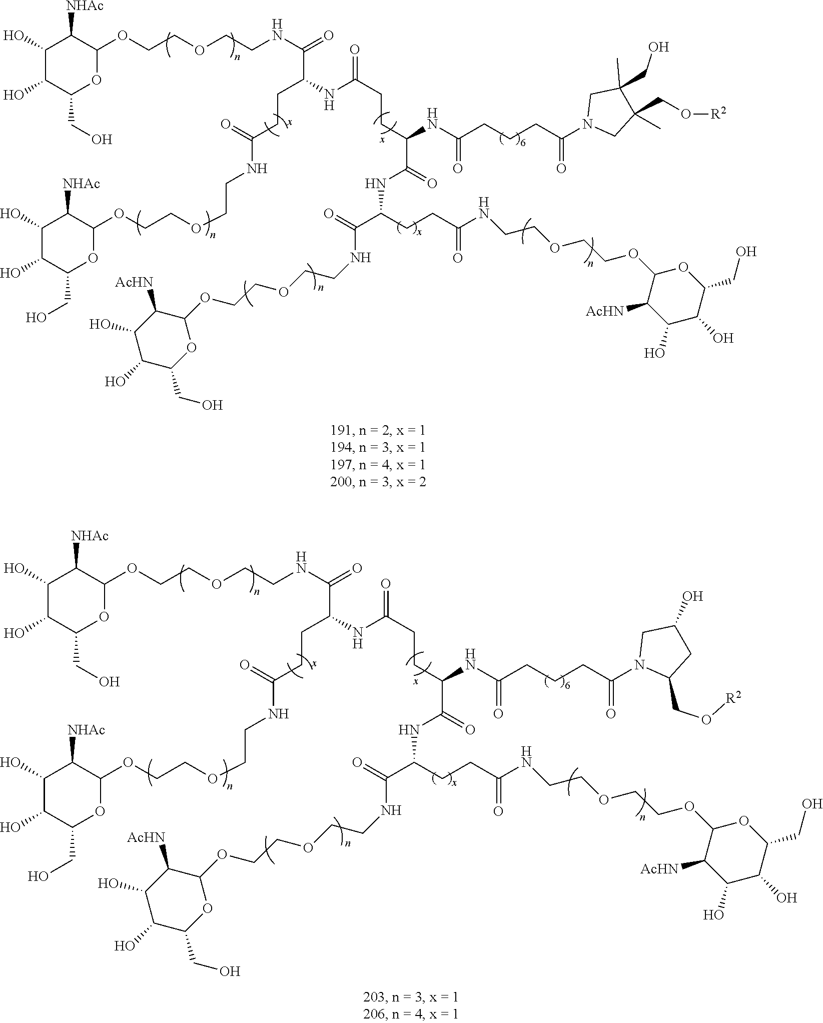

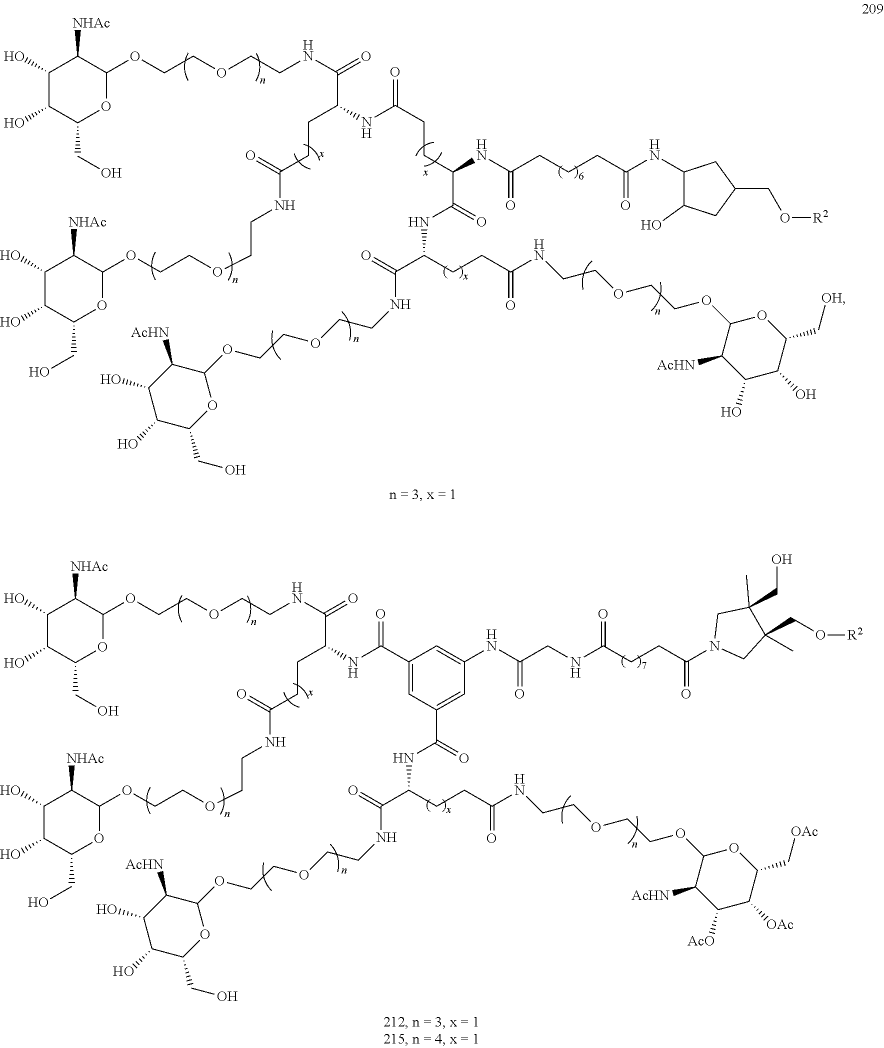

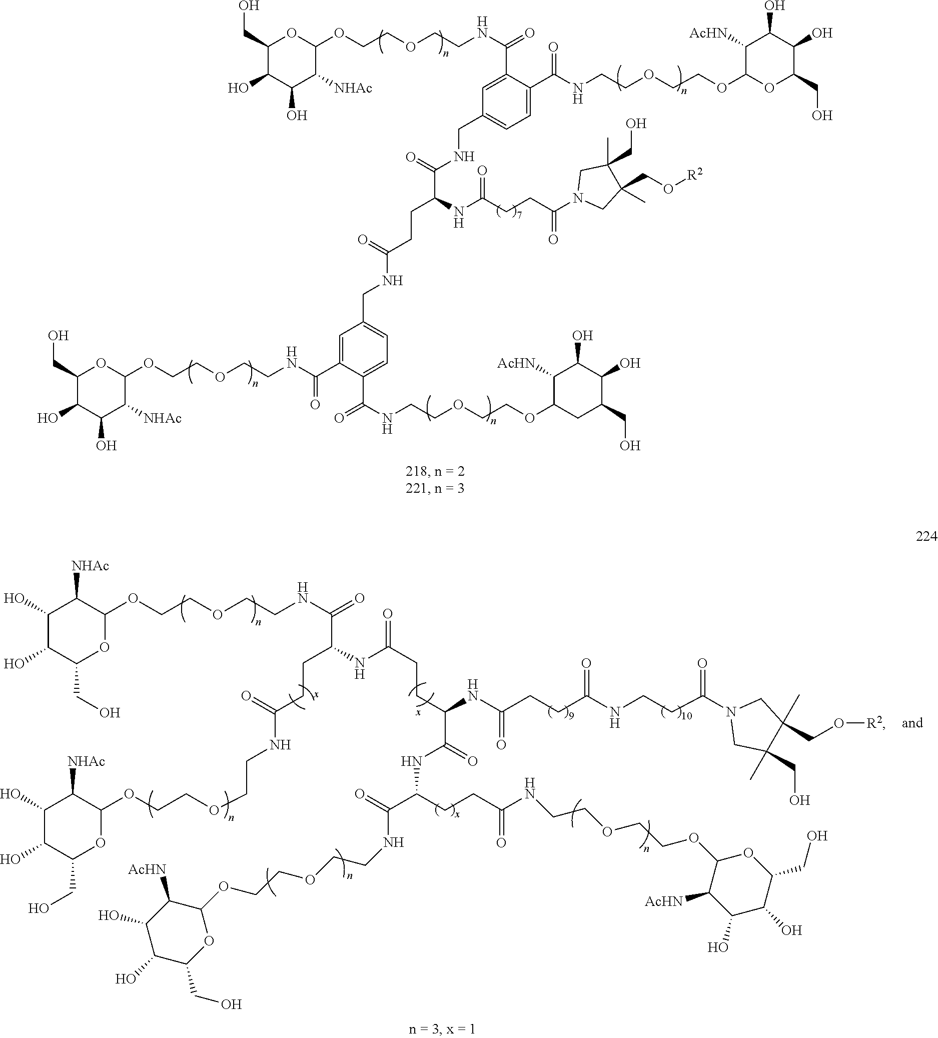

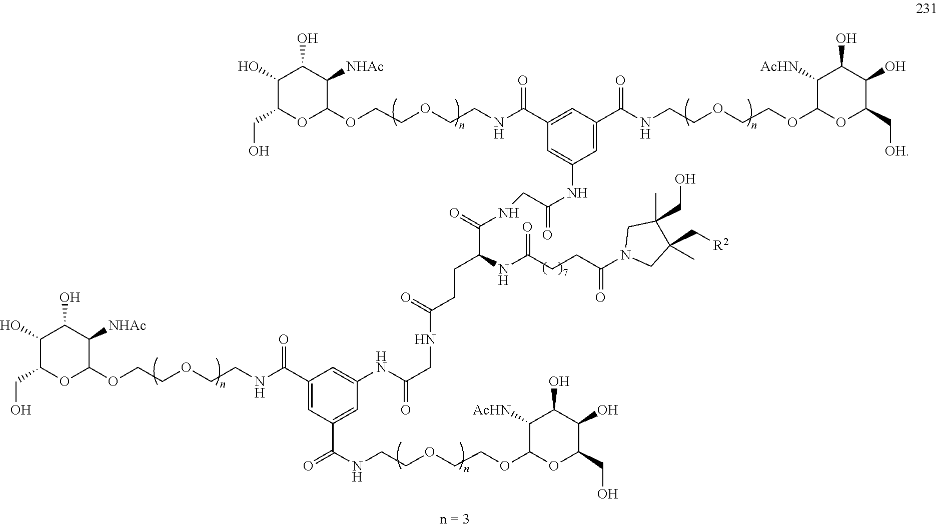

In one embodiment, the compound of formula (I) is a compound formula (Id):

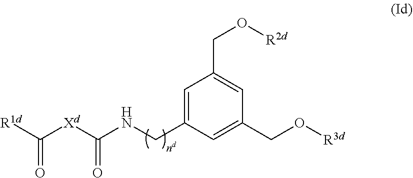

wherein:

R^{1d} is selected from:

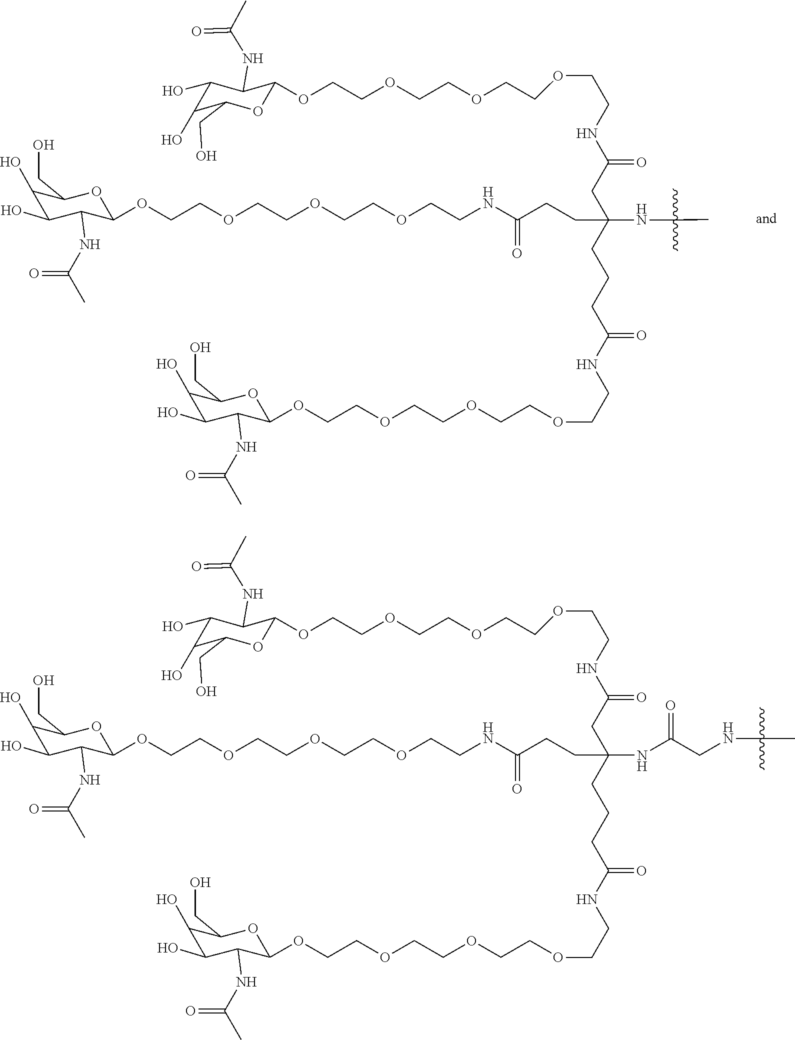

X^{d} is C₂₋₁₀ alkylene;

n^{d} is 0 or 1;

R^{2d} is a nucleic acid; and

R^{3d} is H.

In one embodiment, R^{1d} is:

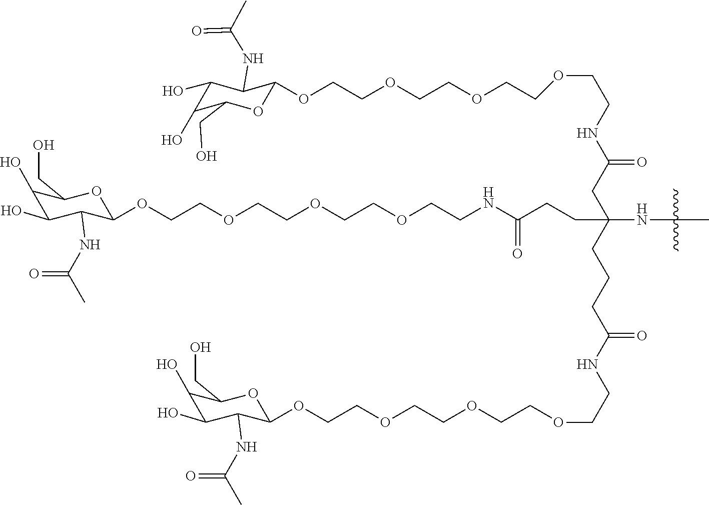 [SMILES: *NC(CCCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CC(=O)NCCOCCOCCOCCOC1OC(CO)C(O)C(O)C1NC(C)=O]

In one embodiment, R^{1d} is:

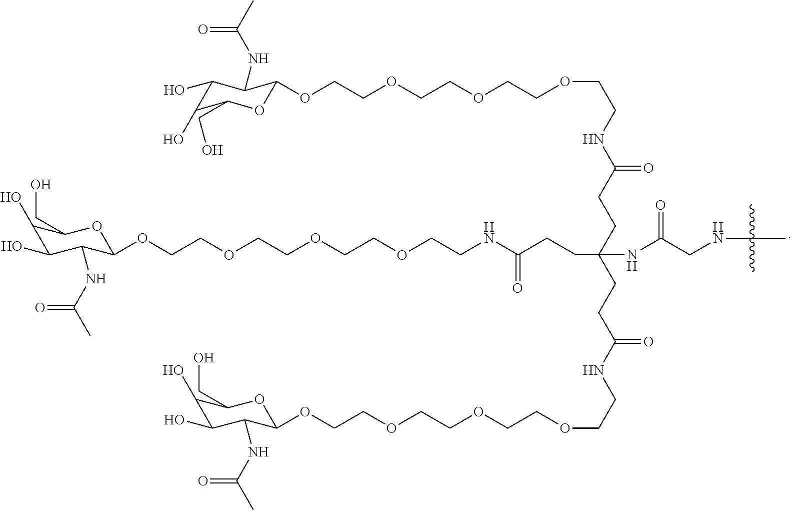 [SMILES: *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

In one embodiment, X^{d} is C₈alkylene.

In one embodiment, n^{d} is 0.

In one embodiment, R^{3d} is H.

In one embodiment, the compound of formula (I) is selected from the group consisting of:

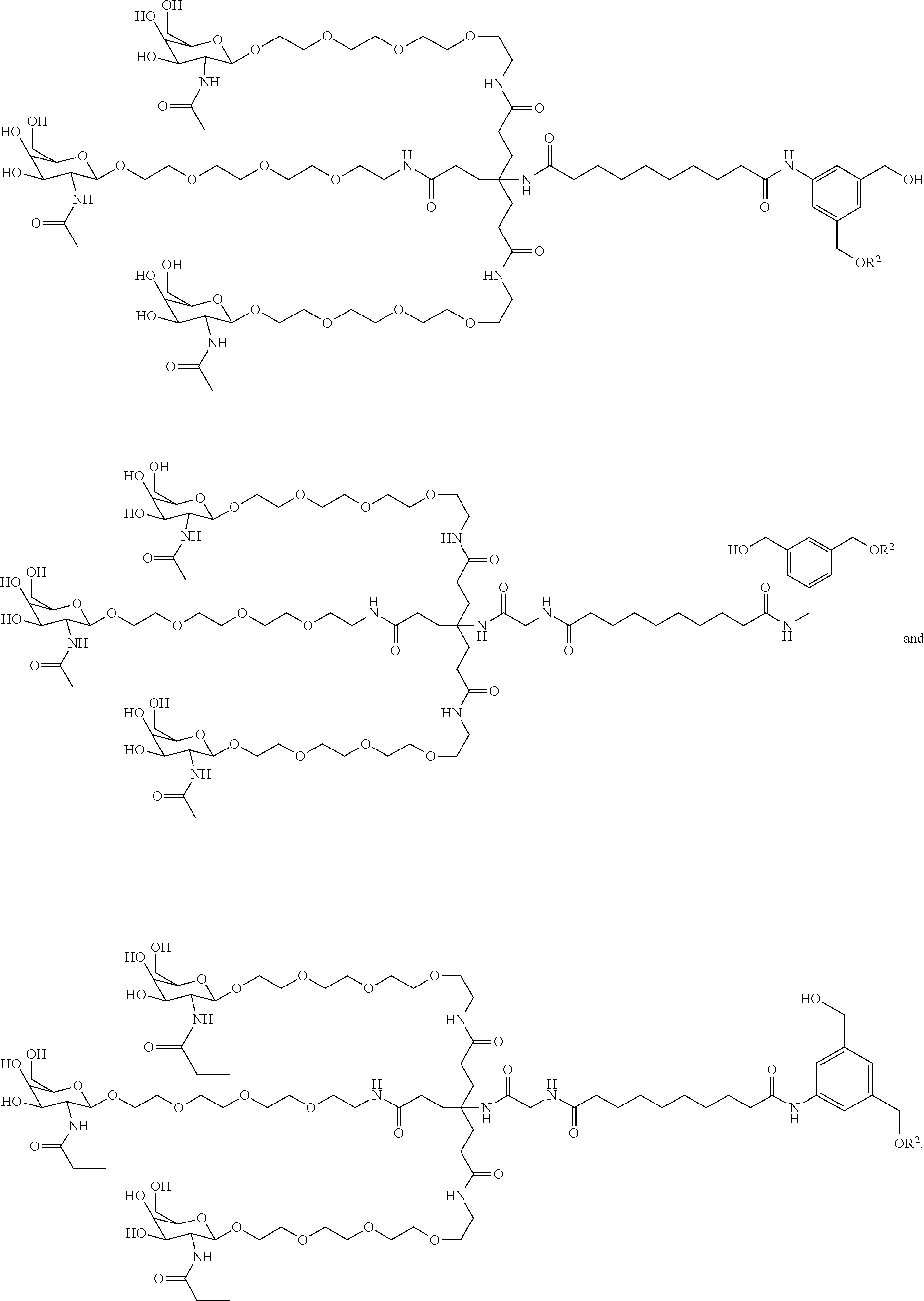

In one embodiment, the compound of formula (I) is a compound of formula (Ig):

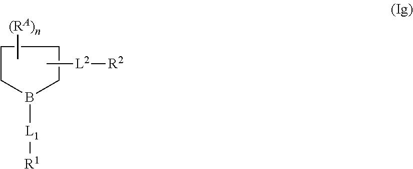

wherein:

B is —N— or —CH—;

L² is C₁₋₄ alkylene-O— that is optionally substituted with hydroxyl or halo; and

n is 0, 1, 2, 3, 4, 5, 6, or 7.

In one embodiment, the compound of formula (I) is selected from the group consisting of:

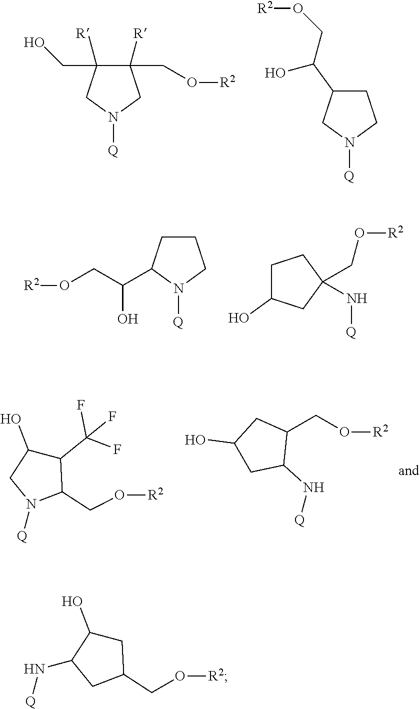

wherein:

Q is -L¹-R¹; and

R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxy.

In one embodiment, the compound of formula (I) is selected from the group consisting of:

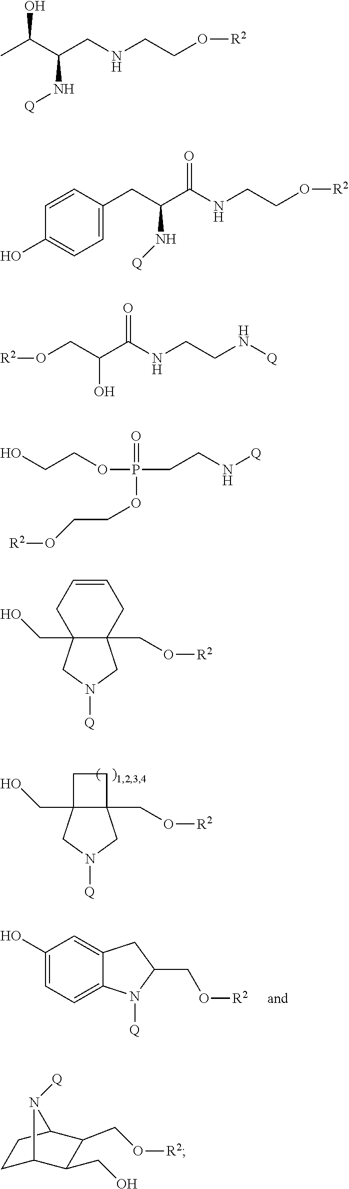

wherein: Q is -L¹-R¹.

In one embodiment, the compound of formula (X) is a compound of formula (XX):

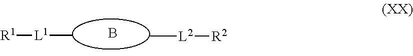

wherein:

R¹ a is targeting ligand;

L¹ is absent or a linking group;

L² is absent or a linking group;

R² is a nucleic acid;

B is divalent and is selected from the group consisting of:

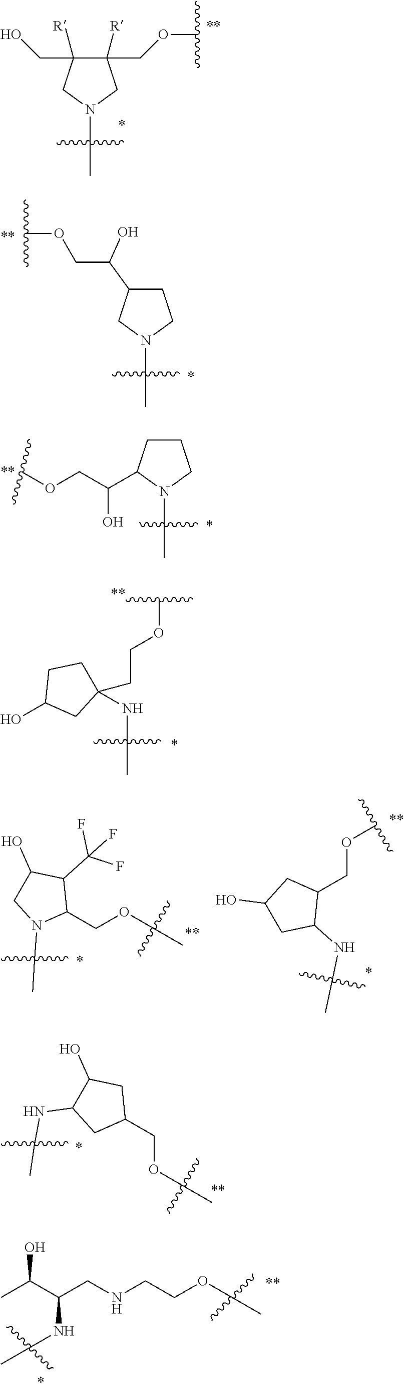

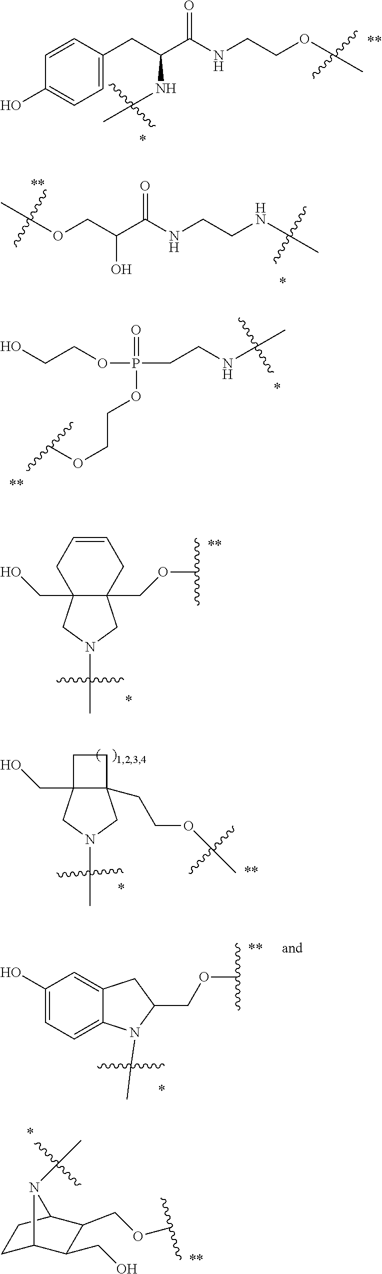

wherein:

each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

the valence marked with * is attached to L¹ or is attached to R¹ if L¹ is absent; and

the valence marked with ** is attached to L² or is attached to R² if L² is absent.

In one embodiment, R¹ comprises 2-8 saccharides.

In one embodiment, R¹ comprises 2-4 saccharides.

In one embodiment, R¹ comprises 3-8 saccharides.

In one embodiment, R¹ comprises 3-6 saccharides.

In one embodiment, R¹ comprises 3-4 saccharides.

In one embodiment, R¹ comprises 2 saccharides.

In one embodiment, R¹ comprises 3 saccharides.

In one embodiment, R¹ comprises 4 saccharides.

In one embodiment, R¹ has the following formula:

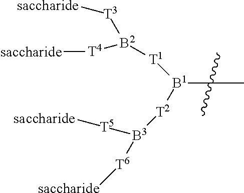

wherein:

B¹ is a trivalent group comprising about 1 to about 20 atoms and is covalently bonded to L¹, T¹, and T².

B² is a trivalent group comprising about 1 to about 20 atoms and is covalently bonded to T¹, T³, and T⁴;

B³ is a trivalent group comprising about 1 to about 20 atoms and is covalently bonded to T², T⁵, and T⁶;

T¹ is absent or a linking group;

T² is absent or a linking group;

T³ is absent or a linking group;

T⁴ is absent or a linking group;

T⁵ is absent or a linking group; and

T⁶ is absent or a linking group.

In one embodiment, each saccharide is independently selected from:

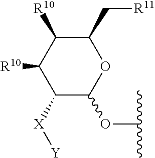

wherein:

X is NR³, and Y is selected from —(C═O)R⁴, —SO₂R⁵, and —(C═O)NR⁶R⁷; or X is —(C═O)— and Y is NR⁸R⁹;

R³ is hydrogen or (C₁-C₄)alkyl;

R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;

R¹⁰ is —OH, —NR⁸R⁹ or —F; and

R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

In one embodiment, each saccharide is independently selected from the group consisting of:

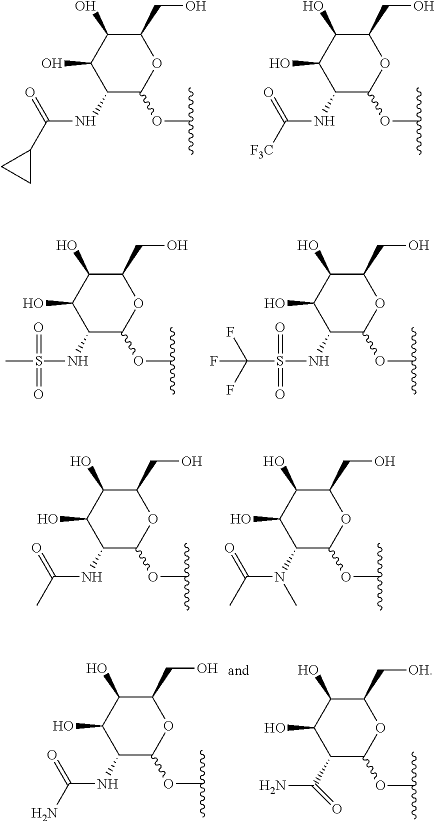

In one embodiment, each saccharide is independently:

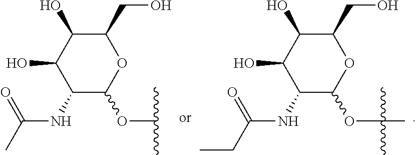 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In one embodiment, one of T¹ and T² is absent.

In one embodiment, both T¹ and T² are absent.

In one embodiment, each of T¹, T², T³, T⁴, T⁵, and T⁶ is independently absent or a branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}- or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment, each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}- or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment, each of T¹, T², T³, T⁴, T⁵, and T⁶ is independently absent or a branched or unbranched, saturated or unsaturated, hydrocarbon chain, having from 1 to 50 carbon atoms, or a salt thereof, wherein one or more of the carbon atoms in the hydrocarbon chain is optionally replaced by —O— or —NR^{X}—, and wherein R^{X} is hydrogen or (C₁-C₆)alkyl, and wherein the hydrocarbon chain, is optionally substituted with one or more (e.g. 1, 2, 3, or 4) substituents selected from halo, hydroxy, and oxo (═O).

In one embodiment, each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

In one embodiment, each of T¹, T², T³, T⁴, T³, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

In one embodiment, at least one of T³, T⁴, T³, and T⁶ is:

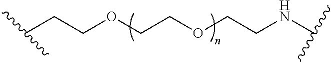

wherein:

n=1, 2, 3.

In one embodiment, each of T³, T⁴, T³, and T⁶ is independently selected from the group consisting of:

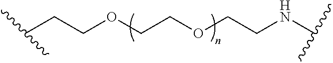

wherein:

n=1, 2, 3.

In one embodiment, at least one of T¹ and T² is glycine.

In one embodiment, each of T¹ and T² is glycine.

In one embodiment, B¹ is a trivalent group comprising 1 to 15 atoms and is covalently bonded to L¹, T¹, and T².

In one embodiment, B¹ is a trivalent group comprising 1 to 10 atoms and is covalently bonded to L¹, T¹, and T².

In one embodiment, B¹ comprises a (C₁-C₆)alkyl.

In one embodiment, B¹ comprises a C₃₋₈ cycloalkyl.

In one embodiment, B¹ comprises a silyl group.

In one embodiment, B¹ comprises a D- or L-amino acid.

In one embodiment, B¹ comprises a saccharide.

In one embodiment, B¹ comprises a phosphate group.

In one embodiment, B¹ comprises a phosphonate group.

In one embodiment, B¹ comprises an aryl.

In one embodiment, B¹ comprises a phenyl ring.

In one embodiment, B¹ is a phenyl ring.

In one embodiment, B¹ is CH.

In one embodiment, B¹ comprises a heteroaryl.

In one embodiment, B¹ is selected from:

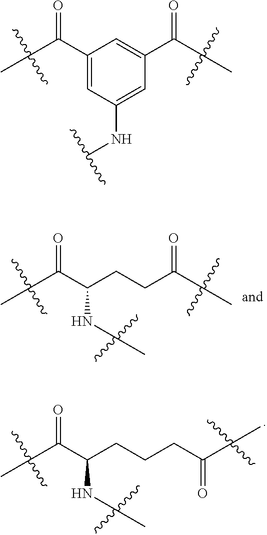 [SMILES: *Nc1cc(C(*)=O)cc(C(*)=O)c1 ; *N[C@@H](CCC(*)=O)C(*)=O ; *N[C@H](CCCC(*)=O)C(*)=O]

In one embodiment, B² is a trivalent group comprising 1 to 15 atoms and is covalently bonded to T², T⁵, and T⁶.

In one embodiment, B² is a trivalent group comprising 1 to 10 atoms and is covalently bonded to T², T⁵, and T⁶.

In one embodiment, B² comprises a (C₁-C₆)alkyl.

In one embodiment, B² comprises a C₃₋₈ cycloalkyl.

In one embodiment, B² comprises a silyl group.

In one embodiment, B² comprises a D- or L-amino acid.

In one embodiment, B² comprises a saccharide.

In one embodiment, B² comprises a phosphate group.

In one embodiment, B² comprises a phosphonate group.

In one embodiment, B² comprises an aryl.

In one embodiment, B² comprises a phenyl ring.

In one embodiment, B² is a phenyl ring.

In one embodiment, B² is CH.

In one embodiment, B² comprises a heteroaryl.

In one embodiment B² is selected from the group consisting of:

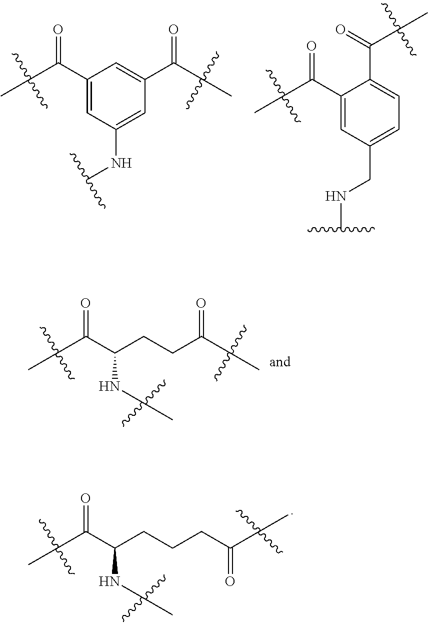 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

In one embodiment, B³ is a trivalent group comprising 1 to 15 atoms and is covalently bonded to L¹, T¹, and T².

In one embodiment, B³ is a trivalent group comprising 1 to 10 atoms and is covalently bonded to L¹, T¹, and T².

In one embodiment, B³ comprises a (C₁-C₆)alkyl.

In one embodiment, B³ comprises a C₃₋₈ cycloalkyl.

In one embodiment, B³ comprises a silyl group.

In one embodiment, B³ comprises a D- or L-amino acid.

In one embodiment, B³ comprises a saccharide.

In one embodiment, B³ comprises a phosphate group.

In one embodiment, B³ comprises a phosphonate group.

In one embodiment, B³ comprises an aryl.

In one embodiment, B³ comprises a phenyl ring.

In one embodiment, B³ is a phenyl ring.

In one embodiment, B³ is CH.

In one embodiment, B³ comprises a heteroaryl.

In one embodiment, B³ is selected from the group consisting of:

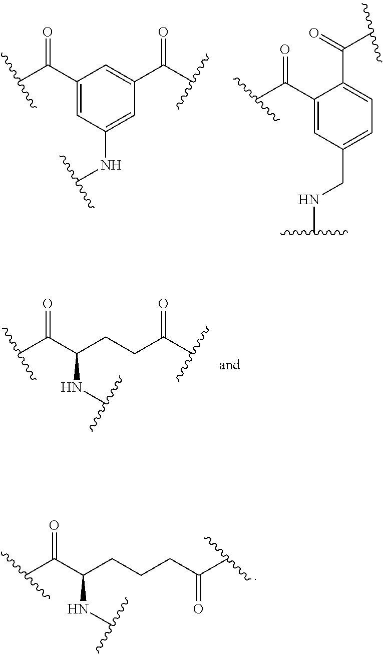 [SMILES: *Nc1cc(C(*)=O)cc(C(*)=O)c1 ; *NCc1ccc(C(*)=O)c(C(*)=O)c1 ; *N[C@H](CCC(*)=O)C(*)=O ; *N[C@H](CCCC(*)=O)C(*)=O]

In one embodiment, B³ is selected from the group consisting of:

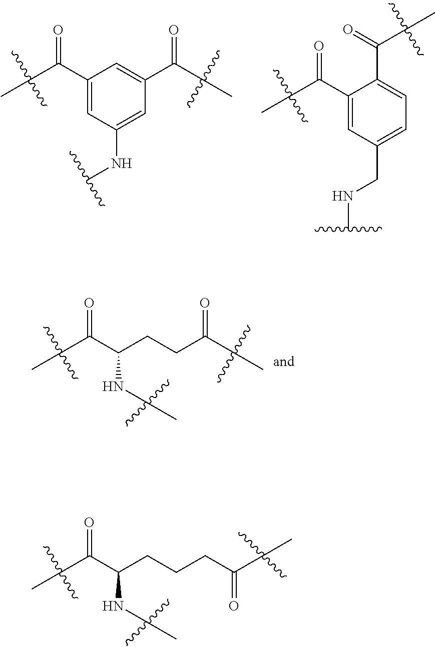 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

or a salt thereof.

In one embodiment, L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

In one embodiment, L¹ is selected from the group consisting of:

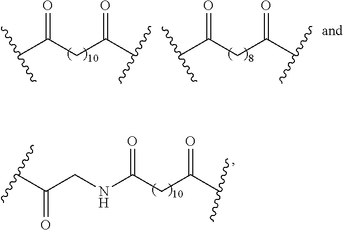

or a salt thereof.

In one embodiment, L¹ is connected to B¹ through a linkage selected from the group consisting of: —O—, —S—, —(C═O)—, —(C═O)—NH—, —NH—(C═O), —(C═O)—O—, —NH—(C═O)—NH—, or —NH—(SO₂)—.

In one embodiment, L¹ is selected from the group consisting of:

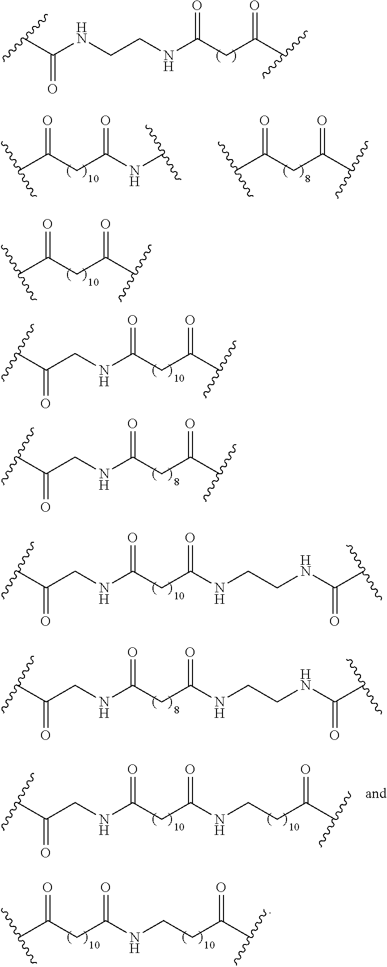

In one embodiment, L² is connected to R² through —O—.

In one embodiment, L² is C₁₋₄ alkylene-O— that is optionally substituted with hydroxy.

In one embodiment, L² is connected to R² through —O—.

In one embodiment, L² is absent.

In one embodiment, the compound of formula (I) is selected from the group consisting of:

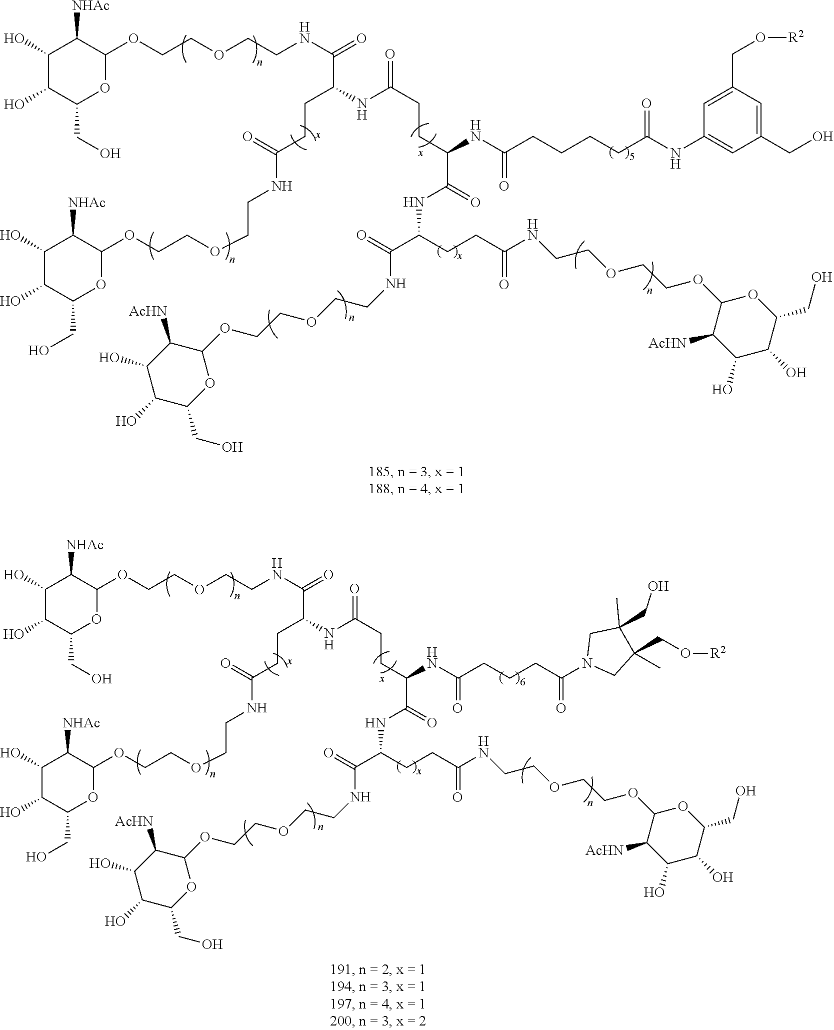

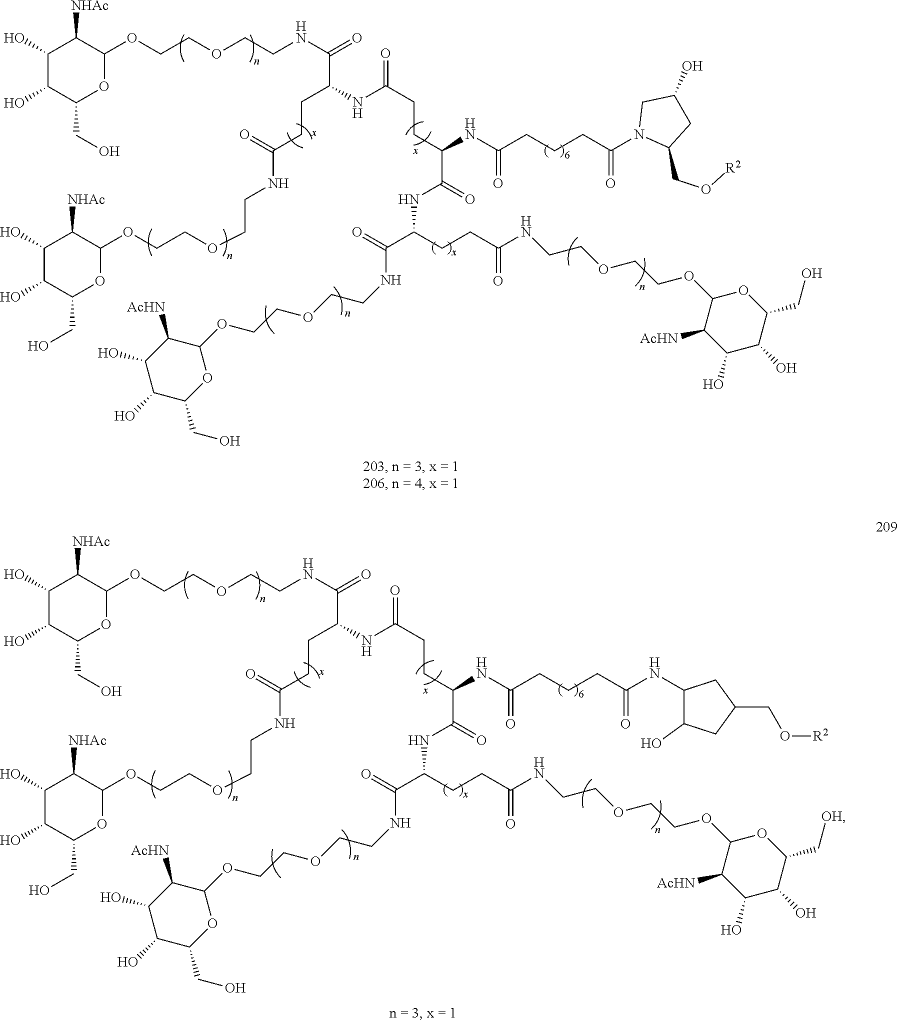

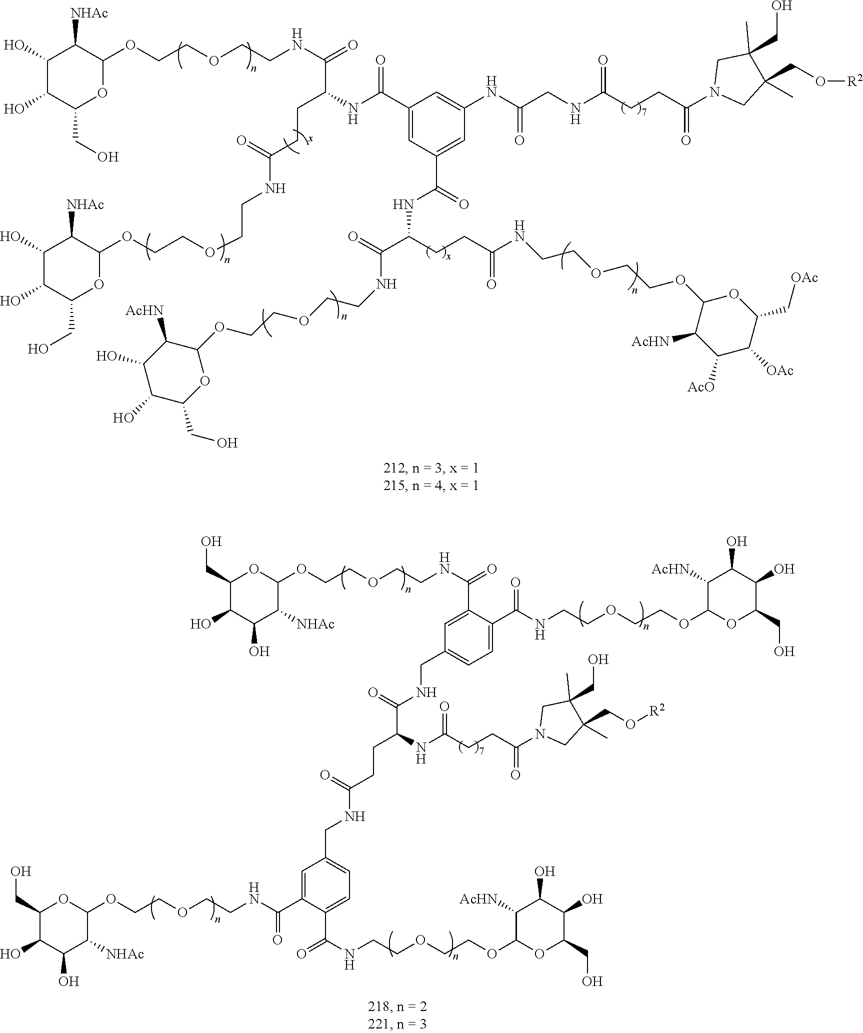

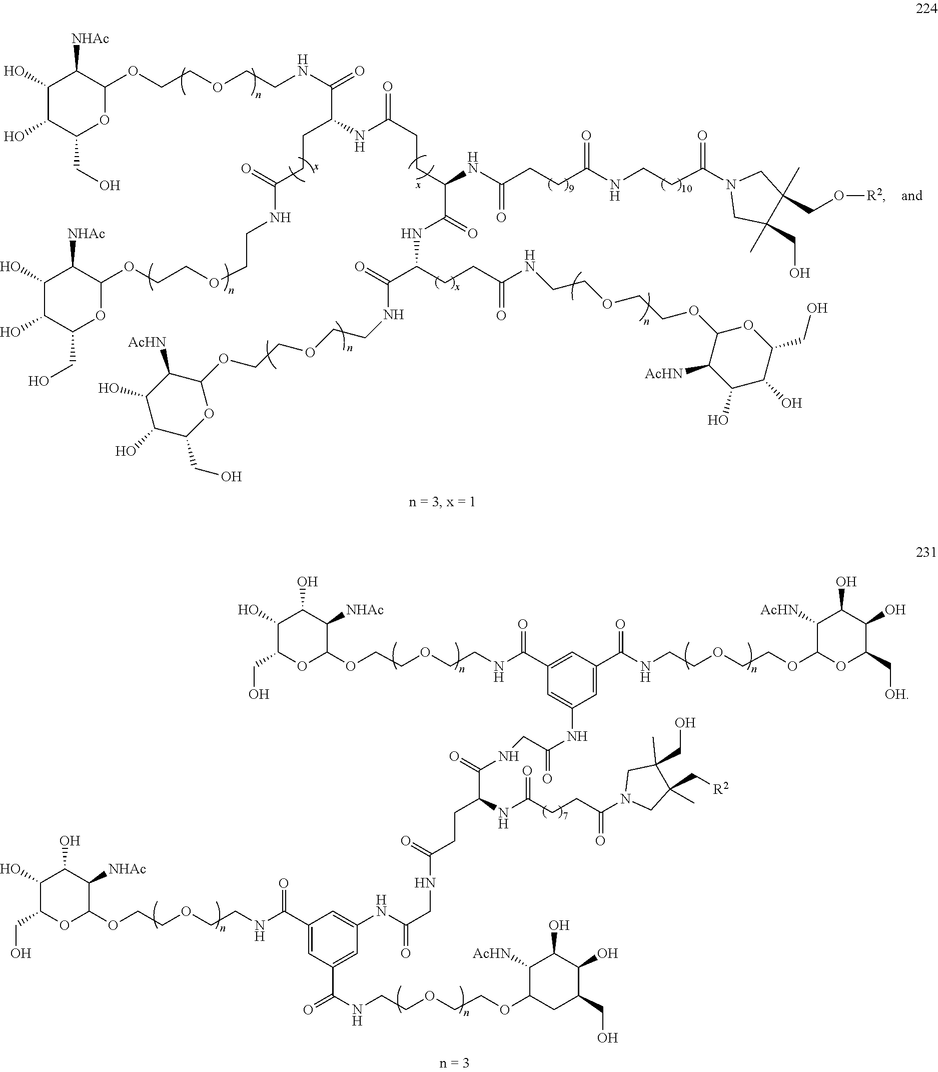

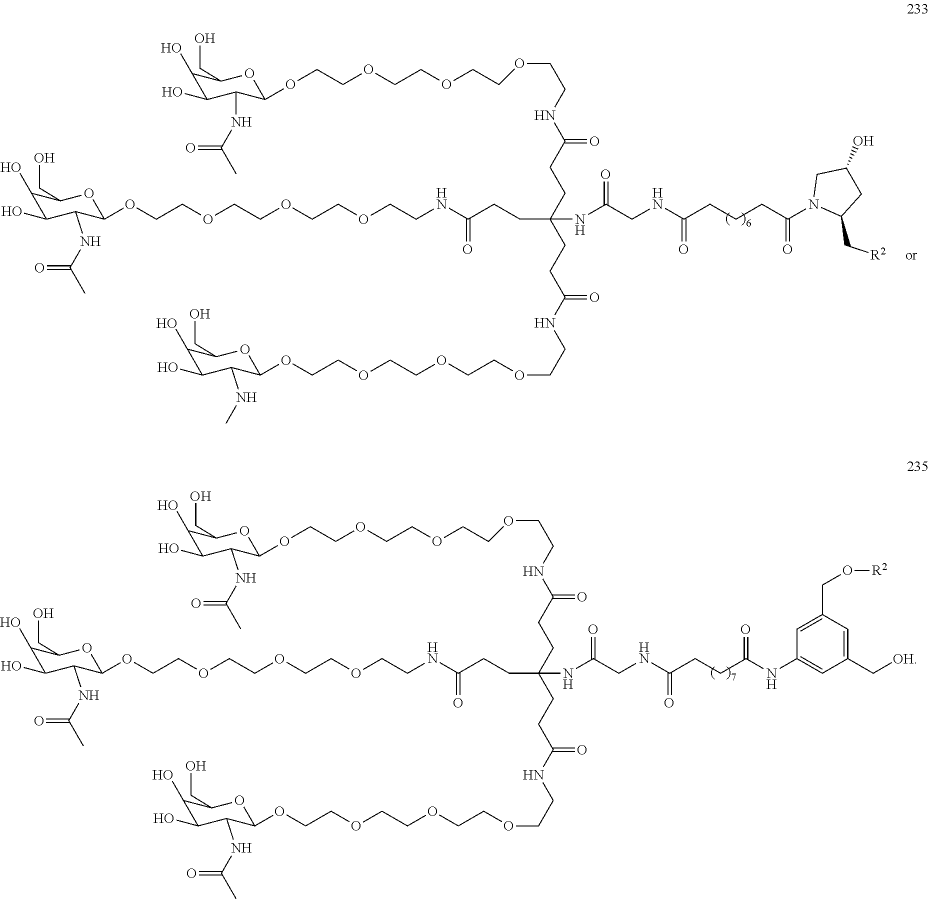

In one embodiment, the compound of formula (I) is the compound,

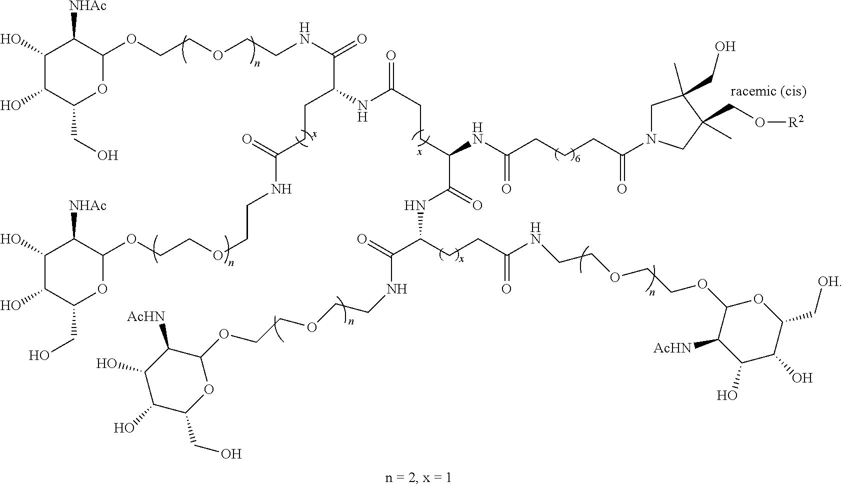

In one embodiment, the compound of formula (I) is the compound,

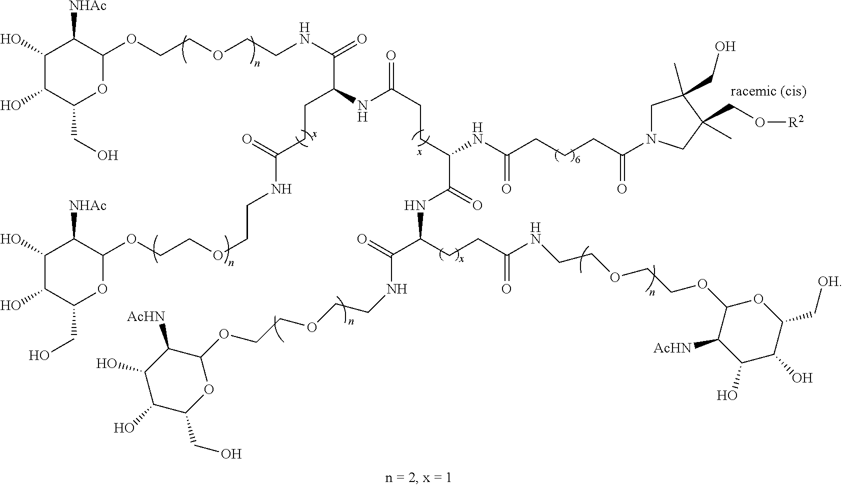

In one embodiment, the compound of formula (I) is the compound,

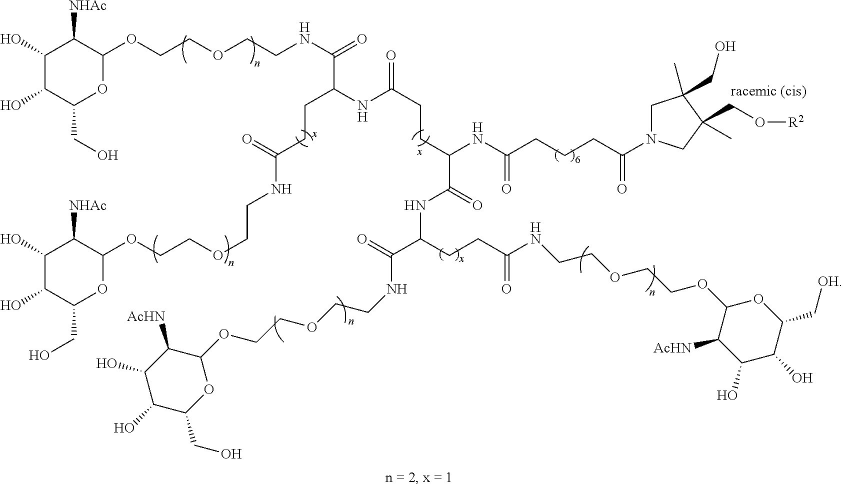

In one embodiment, the compound of formula (I) is the compound,

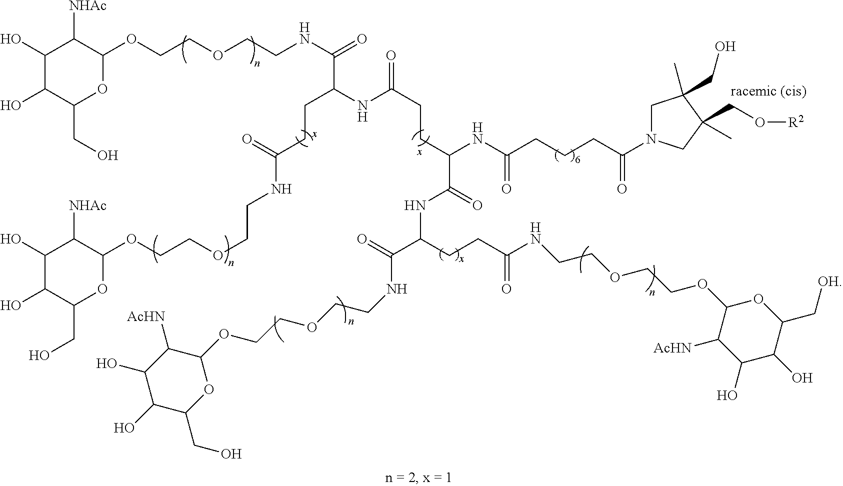

In one embodiment, the compound of formula (I) is the compound,

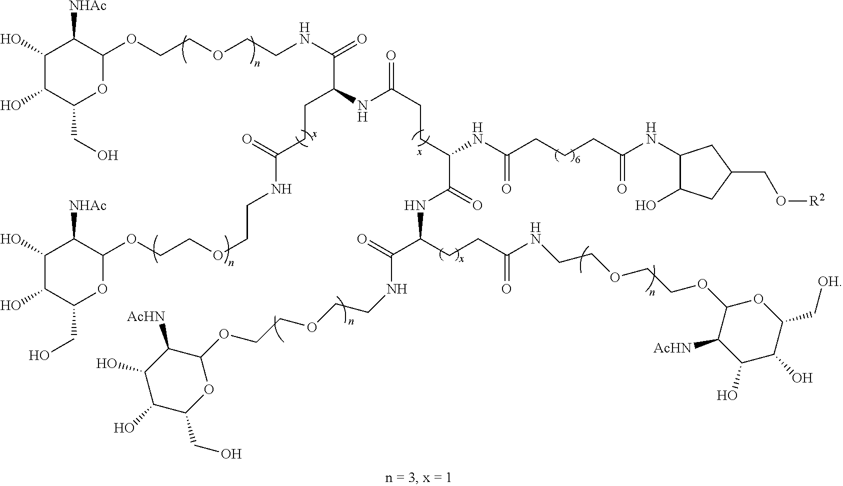

In one embodiment, the compound of formula (I) is the compound,

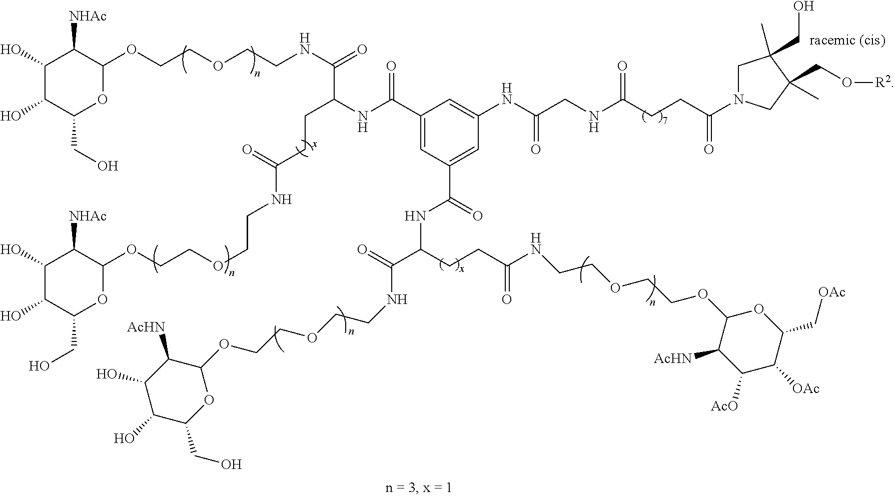

In one embodiment, the compound of formula (I) is the compound,

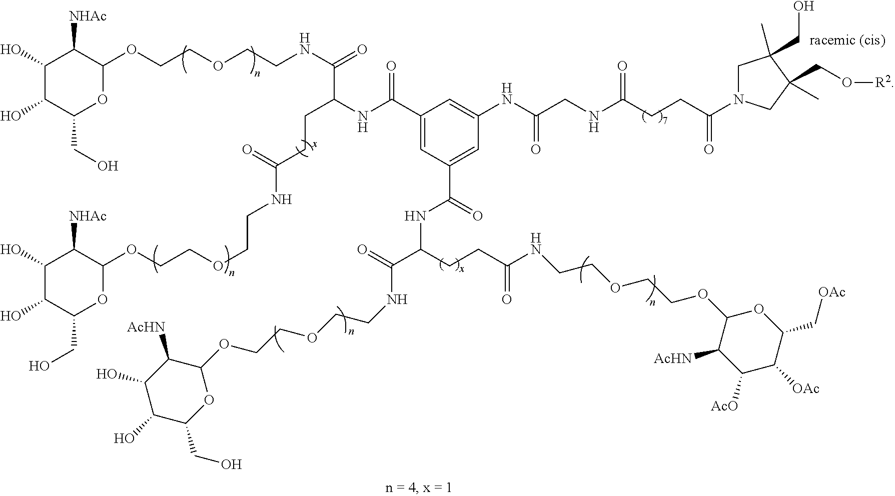

In one embodiment, the compound of formula (I) is the compound,

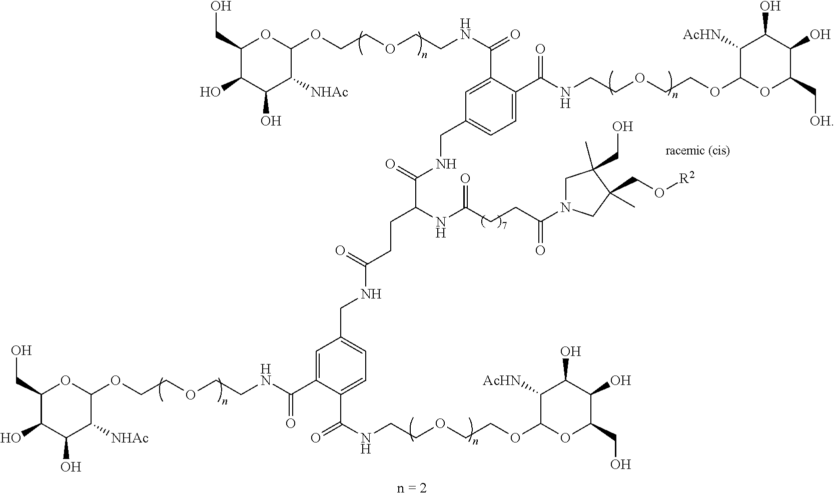

In one embodiment, the compound of formula (I) is the compound,

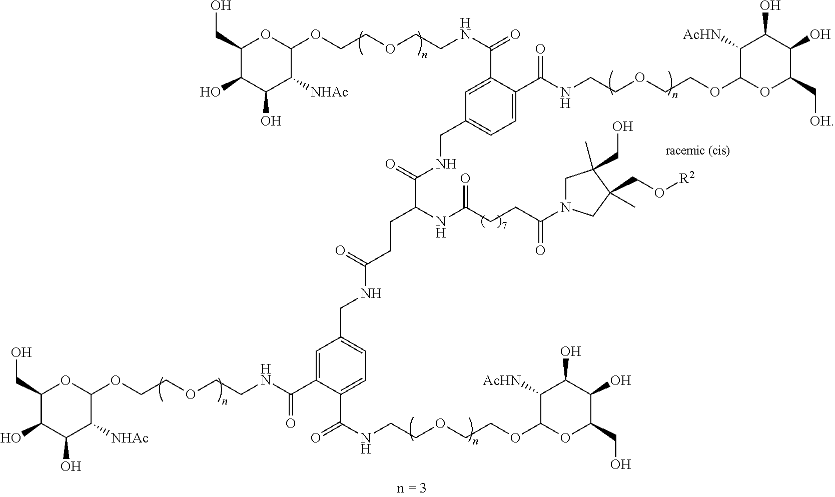

In one embodiment, the compound of formula (I) is the compound,

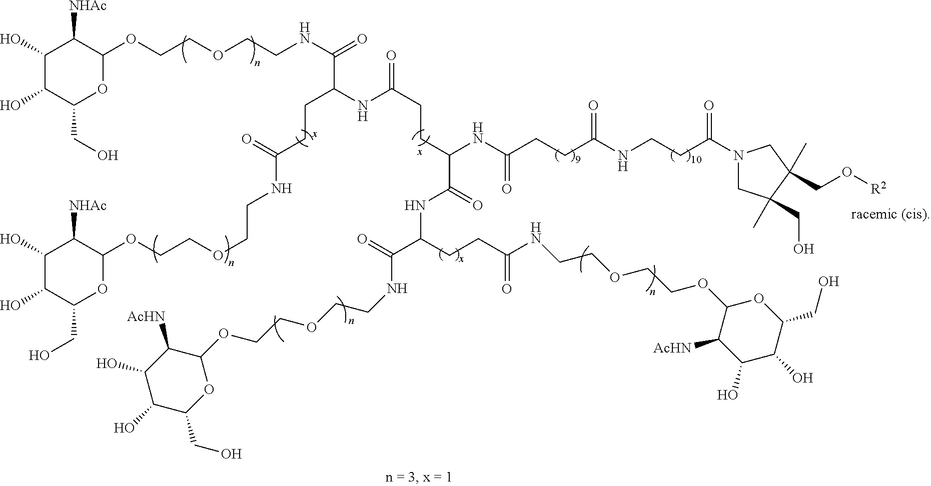

In one embodiment, the compound of formula (I) is the compound,

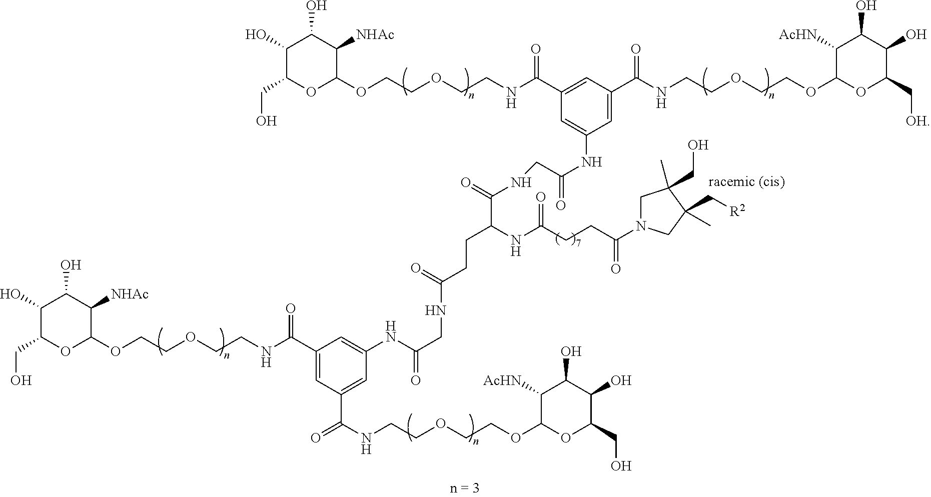

In one embodiment, the compound of formula (I) is the compound,

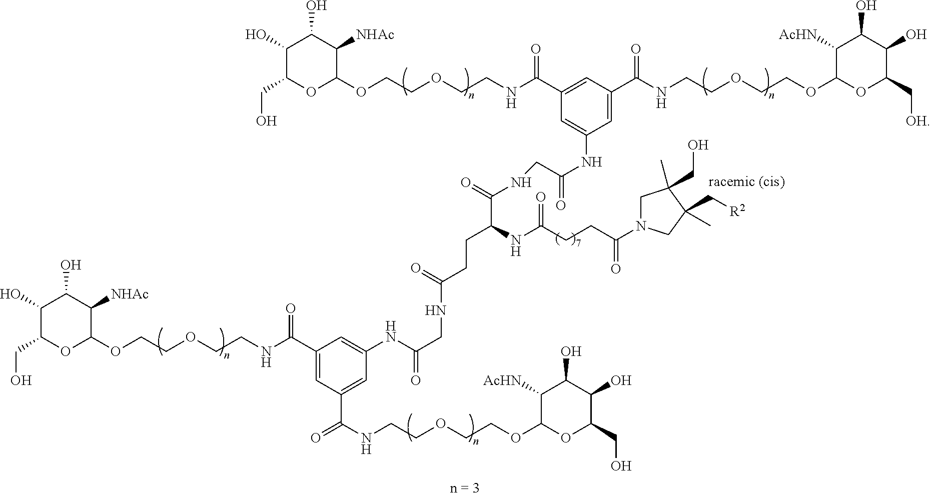

In one embodiment, the compound of formula (I) is the compound,

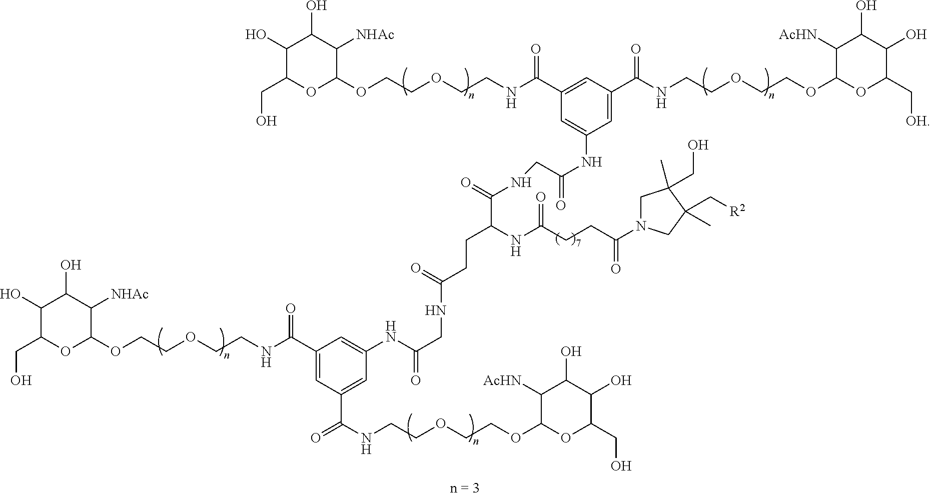

In one embodiment, the compound of formula (I) is the compound,

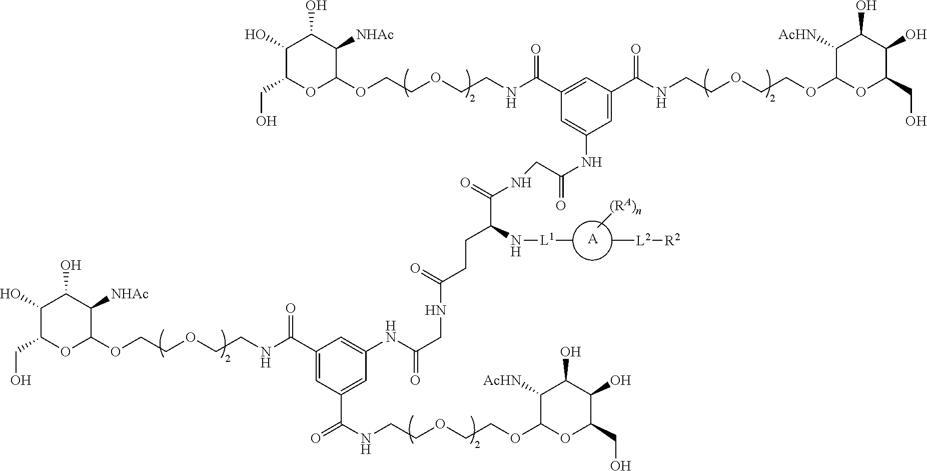

wherein:

L¹ is absent or a linking group;

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10.

In one embodiment, the compound of formula (I) is the compound,

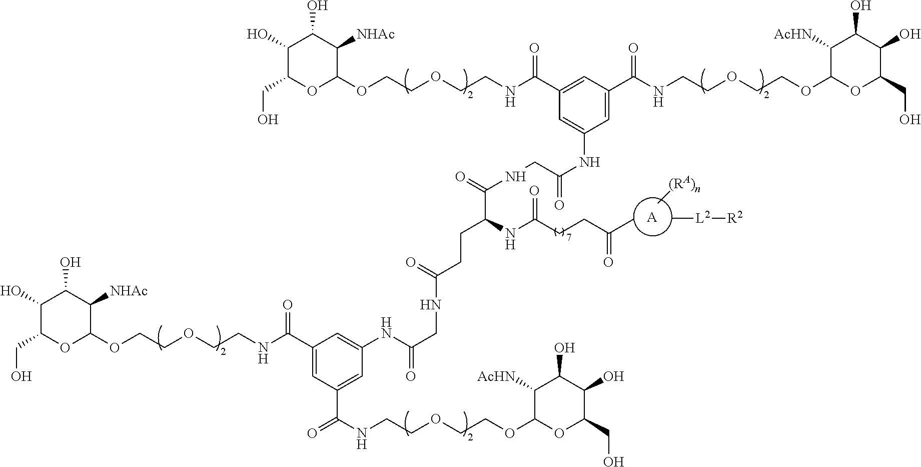

wherein:

L² is absent or a linking group;

R² is a nucleic acid;

the ring A is absent, a 3-20 membered cycloalkyl, a 5-20 membered aryl, a 5-20 membered heteroaryl, or a 3-20 membered heterocycloalkyl;

each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

R^{B} is hydrogen, a protecting group, a covalent bond to a solid support, or a bond to a linking group that is bound to a solid support; and

n is 0, 1, 2, 3, 4, 5, 6, 7, 8, 9, or 10;

or a salt thereof.

In one embodiment, the compound of formula (I) is the compound,

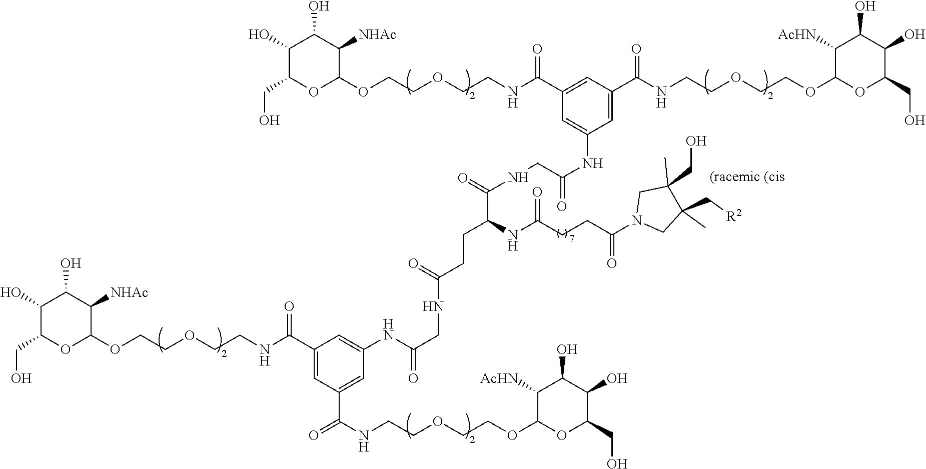

or a salt thereof wherein R² is a nucleic acid.

In one embodiment, the compound of formula (I) is the compound,

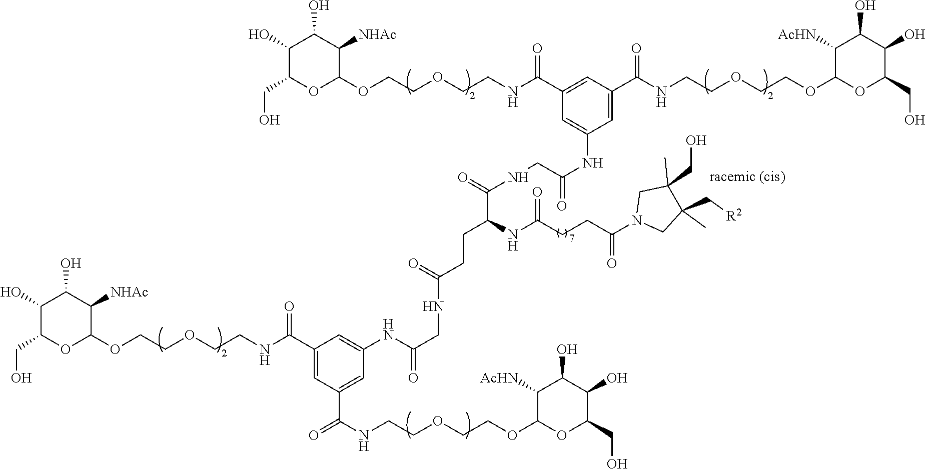

In one embodiment, the compound of formula (I) is the compound,

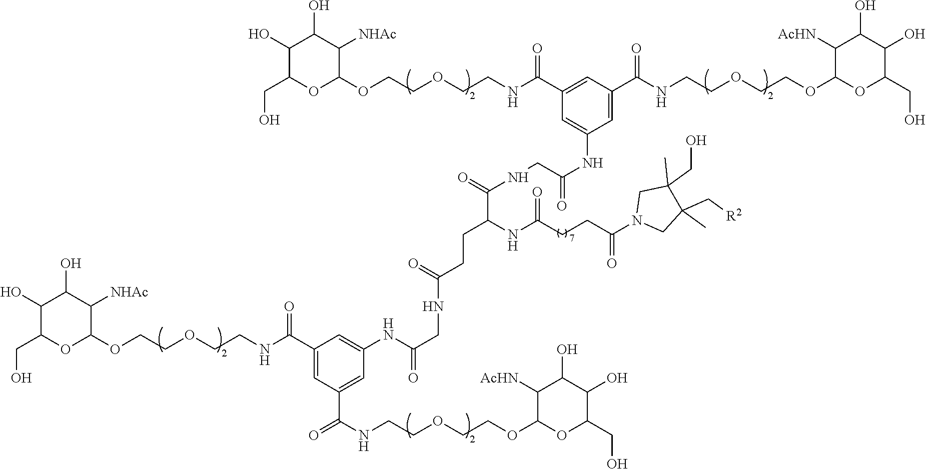

In one embodiment, the compound of formula (I) is the compound,

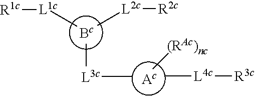

wherein:

R^{1c} is a saccharide;

L^{1c} is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;

B^{c} is a 5-10 membered aryl or a 5-10 membered heteroaryl, which 5-10 membered aryl or 5-10 membered heteroaryl is optionally substituted with one or more groups independently selected from the group consisting of halo, hydroxy, cyano, trifluoromethyl, trifluoromethoxy, (C₁-C₆)alkyl, (C₁-C₆)alkoxy, (C₁-C₆)alkoxycarbonyl, (C₁-C₆)alkanoyloxy, (C₃-C₆)cycloalkyl, and (C₃-C₆)cycloalkyl(C₁-C₆)alkyl

L^{2c} is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;

R^{2c} is a saccharide;

L^{3c} is absent or a linking group;

A^{c} is a 3-20 membered cycloalkyl, a 5-20 membered aryl, a 5-20 membered heteroaryl, or a 3-20 membered heterocycloalkyl;

each R^{Ac} is independently selected from the group consisting of hydrogen, hydroxy, CN, F, Cl, Br, I, —C₁₋₂ alkyl-OR^{a}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;

nc is 0, 1, 2, 3, 4, 5, 6, 7, 8, 9, or 10;

L^{4c} is absent or a linking group;

R^{3c} is a nucleic acid;

R^{ac} is hydrogen; and

L^{5c} is a linking group;

or a salt thereof.

In one embodiment, B^{c} is a 5-10 membered aryl.

In one embodiment, B^{c} is naphthyl or phenyl.

In one embodiment, B^{c} is phenyl.

In one embodiment, the group:

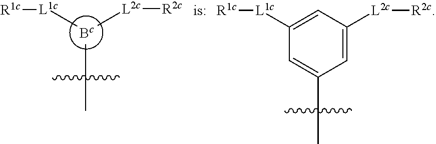

In one embodiment, B^{c} is a 5-10 membered heteroaryl.

In one embodiment, B^{c} is pyridyl, pyrimidyl, quinolyl, isoquinolyl, imidazolyl, thiazolyl, oxadiazolyl or oxazolyl.

In one embodiment, the group:

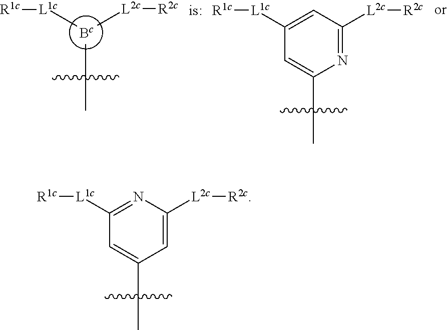

In one embodiment, the group:

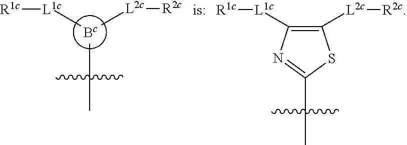

In one embodiment, L^{1c} is a divalent, unbranched, saturated hydrocarbon chain, having from 0 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo.

In one embodiment, L^{1c} is a divalent, unbranched, saturated hydrocarbon chain, having from 0 to 12 carbon atoms, wherein one or more (e.g. 1, 2, 3, or 4) of the carbon atoms in the hydrocarbon chain is optionally replaced by —O—, —NR^{X}—C(═O)—, or —C(═O)—NR^{X}—, and wherein R^{X} is hydrogen or (C₁-C₆)alkyl.

In one embodiment, L^{1c} is:
—C(═O)N(H)—CH₂CH₂OCH₂CH₂OCH₂CH₂—,
—C(═O)N(H)—CH₂CH₂OCH₂CH₂OCH₂CH₂OCH₂CH₂—,
—C(═O)N(CH₃)—CH₂CH₂OCH₂CH₂OCH₂CH₂—, or
—C(═O)N(CH₃)—CH₂CH₂OCH₂CH₂OCH₂CH₂OCH₂CH₂—.

In one embodiment, L^{2c} is a divalent, unbranched, saturated hydrocarbon chain, having from 0 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo.

In one embodiment, L^{2c} is a divalent, unbranched, saturated hydrocarbon chain, having from 0 to 12 carbon atoms, wherein one or more (e.g. 1, 2, 3, or 4) of the carbon atoms in the hydrocarbon chain is optionally replaced by —O—, —NR^{X}—C(═O)—, or —C(═O)—NR^{X}—, and wherein R^{X} is hydrogen or (C₁-C₆)alkyl.

In one embodiment, L^{2c} is:
—C(═O)N(H)—CH₂CH₂OCH₂CH₂OCH₂CH₂—,
—C(═O)N(H)—CH₂CH₂OCH₂CH₂OCH₂CH₂OCH₂CH₂—,
—C(═O)N(CH₃)—CH₂CH₂OCH₂CH₂OCH₂CH₂—, or
—C(═O)N(CH₃)—CH₂CH₂OCH₂CH₂OCH₂CH₂OCH₂CH₂—.

In one embodiment, R^{1c} is:

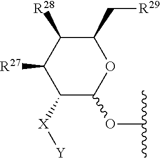

wherein:

X is NR²⁰ and Y is selected from —(C═O)R²¹, —SO₂R², and —(C═O)NR²³R²⁴; or X is —(C═O)— and Y is NR²⁵R²⁶; or X is —NR³⁷R³⁸ and Y is absent

R²⁰ is hydrogen or (C₁-C₄)alkyl;

R²¹, R²², R²³, R²⁴, R²⁵ and R²⁶ are each independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo, (C₁-C₄)alkyl, and (C₁-C₄)alkoxy;

R²⁷ is —OH, —NR²⁵R²⁶ or —F;

R²⁸ is —OH, —NR²⁵R²⁶ or —F;

R²⁹ is —OH, —NR²⁵R²⁶, —F, —N₃, —NR³⁵R³⁶, or 5 membered heterocycle that is optionally substituted with one or more groups independently selected from the group consisting of halo, hydroxyl, carboxyl, amino, (C₁-C₄)alkyl, aryl, and (C₁-C₄)alkoxy, wherein any (C₁-C₄)alkyl, and (C₁-C₄)alkoxy is optionally substituted with one or more groups independently selected from the group consisting of halo, and wherein any aryl is optionally substituted with one or more groups independently selected from the group consisting of halo, hydroxyl, nitro, cyano, amino, (C₁-C₈)alkyl, (C₁-C₈)alkoxy, (C₁-C₈)alkanoyl, (C₁-C₈)alkoxycarbonyl, (C₁—C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy;

each R³⁵ and R³⁶ is independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo and (C₁-C₄)alkoxy; or R³⁵ and R³⁸ taken together with the nitrogen to which they are attached form a 5-6 membered heteroaryl ring, which heteroaryl ring is optionally substituted with one or more groups independently selected from the group consisting of (C₁-C₈)alkyl, (C₁-C₈)alkoxy, aryl, and (C₃-C₆)cycloalkyl, wherein any aryl, and (C₃-C₆)cycloalkyl is optionally substituted with one or more groups R³⁹;

each R³⁷ and R³⁸ is independently selected from the group consisting of hydrogen, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy; or R³⁷ and R³⁸ taken together with the nitrogen to which they are attached form a 5-8 membered heterocycle that is optionally substituted with one or more groups independently selected from the group consisting of halo, hydroxyl, carboxyl, amino, oxo (═O), (C₁-C₄)alkyl, and (C₁-C₄)alkoxy, wherein any (C₁-C₄)alkyl, and (C₁-C₄)alkoxy is optionally substituted with one or more groups independently selected from halo; and

each R³⁹ is independently selected from the group consisting of (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from halo.

In one embodiment, R^{1c} is:

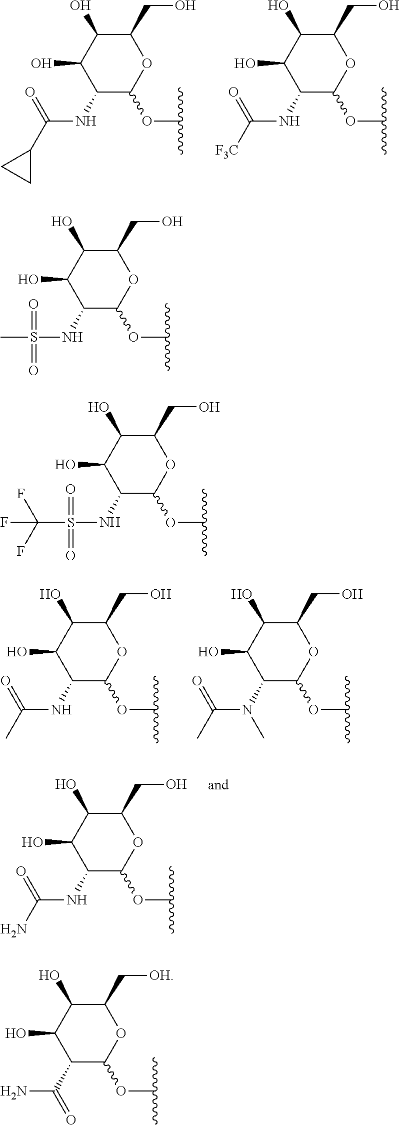

In one embodiment, R^{1c} is:

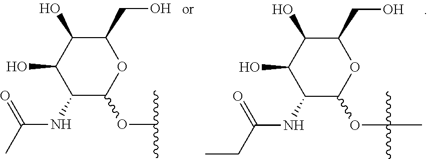 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

In one embodiment, R^{1c} is:

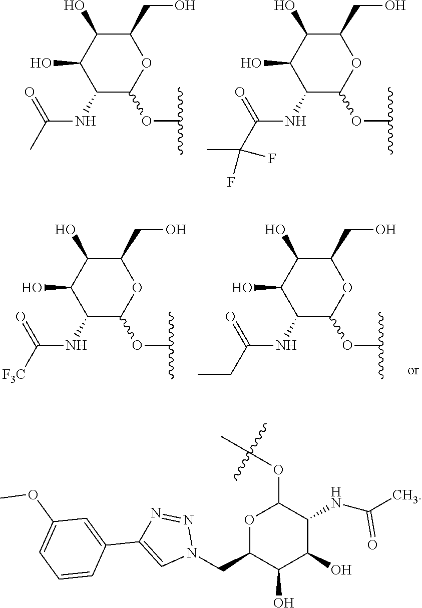

In one embodiment, R^{1c} is

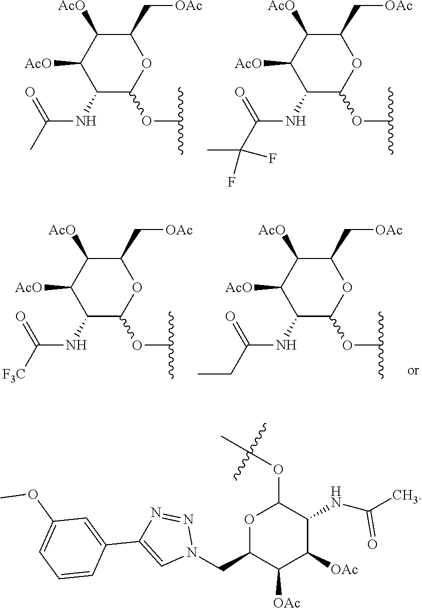

In one embodiment, R^{2c} is:

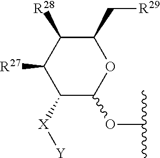

wherein:

X is NR²⁰ and Y is selected from —(C═O)R²¹, —SO₂R²², and —(C═O)NR²³R²⁴; or X is —(C═O)— and Y is NR²⁵R²⁶; or X is —NR³⁷R³⁸ and Y is absent

R²⁰ is hydrogen or (C₁-C₄)alkyl;

R²¹, R²², R²³, R²⁴, R²¹ and R²⁶ are each independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo, (C₁-C₄)alkyl, and (C₁-C₄)alkoxy;

R²⁷ is —OH, —NR²⁵R²⁶ or —F;

R²⁸ is —OH, —NR²⁵R²⁶ or —F;

R²⁹ is —OH, —NR²⁵R²⁶, —F, —N₃, —NR³⁵R³⁶, or 5 membered heterocycle that is optionally substituted with one or more groups independently selected from the group consisting of halo, hydroxyl, carboxyl, amino, (C₁-C₄)alkyl, aryl, and (C₁-C₄)alkoxy, wherein any (C₁-C₄)alkyl, and (C₁-C₄)alkoxy is optionally substituted with one or more groups independently selected from the group consisting of halo, and wherein any aryl is optionally substituted with one or more groups independently selected from the group consisting of halo, hydroxyl, nitro, cyano, amino, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy;

each R³⁵ and R³⁶ is independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo and (C₁-C₄)alkoxy; or R³⁵ and R³⁶ taken together with the nitrogen to which they are attached form a 5-6 membered heteroaryl ring, which heteroaryl ring is optionally substituted with one or more groups independently selected from the group consisting of (C₁-C₈)alkyl, (C₁-C₈)alkoxy, aryl, and (C₃-C₆)cycloalkyl, wherein any aryl, and (C₃-C₆)cycloalkyl is optionally substituted with one or more groups R³⁹;

each R³⁷ and R³⁸ is independently selected from the group consisting of hydrogen, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy; or R³⁷ and R³⁸ taken together with the nitrogen to which they are attached form a 5-8 membered heterocycle that is optionally substituted with one or more groups independently selected from the group consisting of halo, hydroxyl, carboxyl, amino, oxo (═O), (C₁-C₄)alkyl, and (C₁-C₄)alkoxy, wherein any (C₁-C₄)alkyl, and (C₁-C₄)alkoxy is optionally substituted with one or more groups independently selected from halo; and

each R³⁹ is independently selected from the group consisting of (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from halo.

In one embodiment, R^{2c} is:

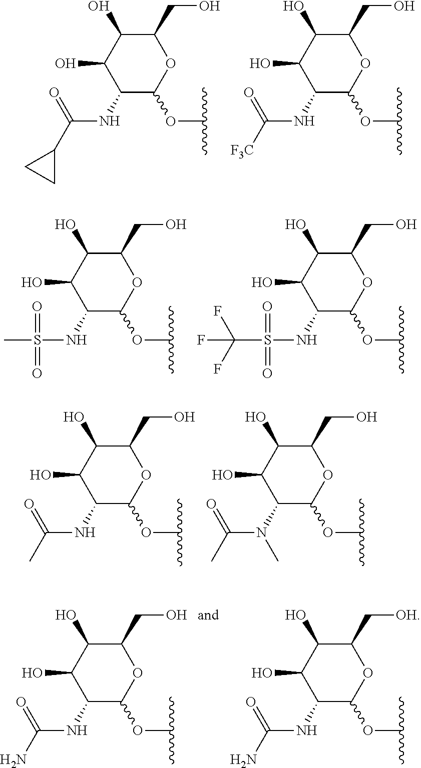

In one embodiment, R^{2c} is:

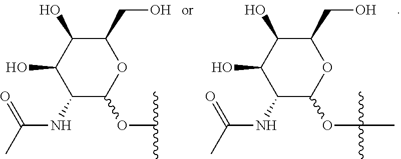 [SMILES: *OC1O[C@H](CO)[C@H](O)[C@H](O)[C@H]1NC(C)=O ; *OC1O[C@H](CO)[C@H](O)[C@H](O)[C@H]1NC(C)=O]

In one embodiment, R^{2c} is:

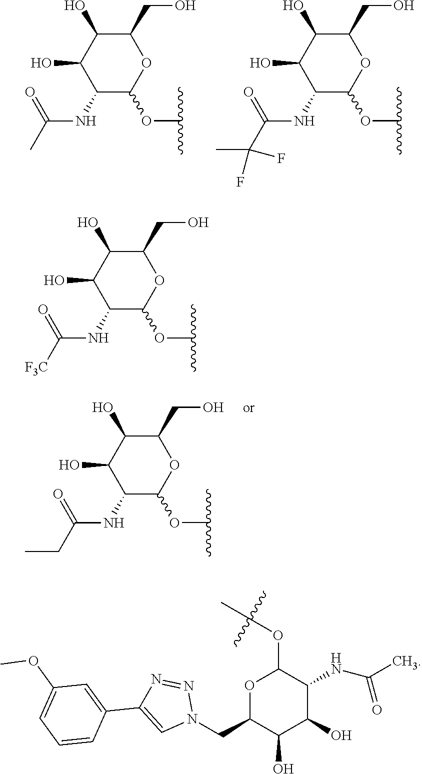

In one embodiment, R^{2c} is

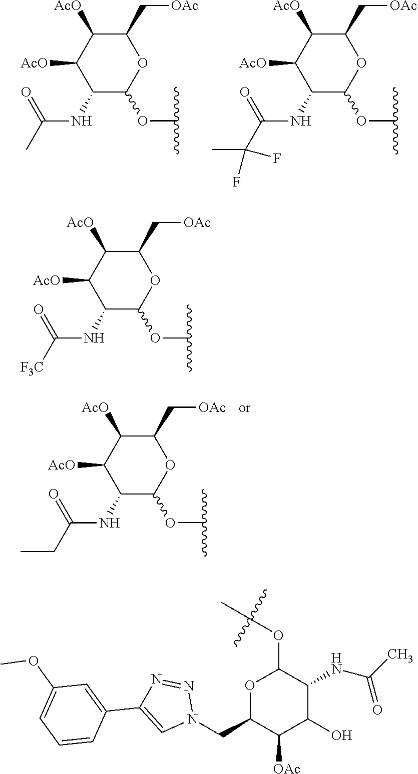

In one embodiment, L^{3c} is a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (0), carboxy, aryl, aryloxy, heteroaryl, and heteroaryloxy.

In one embodiment, L^{3c} is a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)Cycloalkyl, (C₁-C₆)alkanoyl, (C₁-C₆)alkanoyloxy, (C₁—C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment, L^{3c} is a divalent, branched or unbranched, saturated or unsaturated, hydrocarbon chain, having from 1 to 30 carbon atoms, wherein one or more of the carbon atoms is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more halo or oxo (═O).

In one embodiment, L^{3c} is:

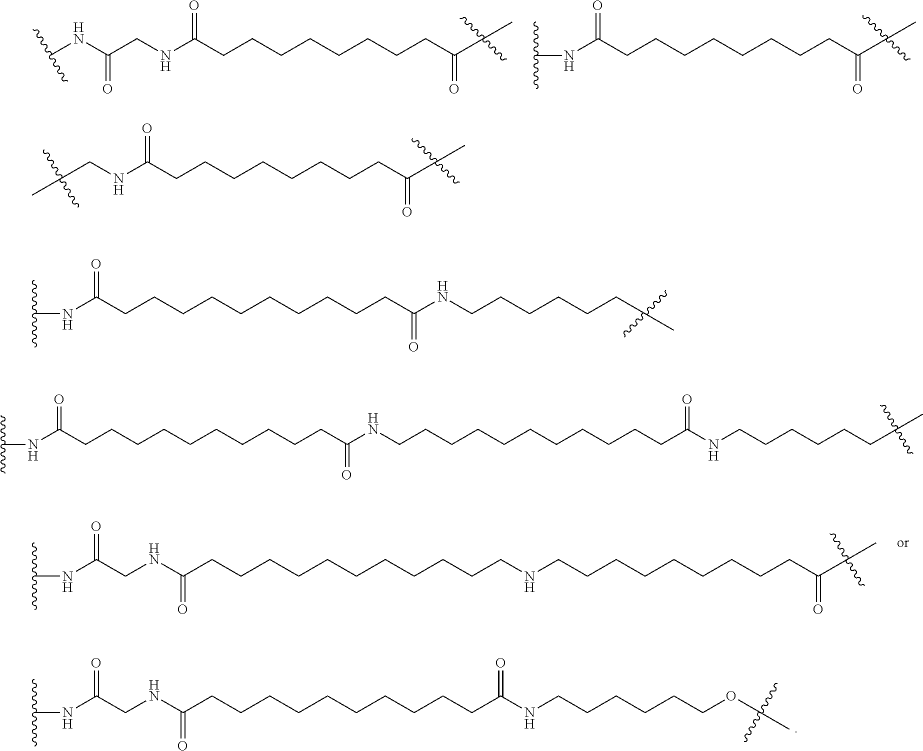

In one embodiment, L^{3c} is connected to B through —NH—, —O—, —S—, —(C═O)—, —(C═O)—NH—, —NH—(C═O)—, —(C═O)—O—, —NH—(C═O)—NH—, or —NH—(SO)—.

In one embodiment, L^{4c} is a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment, L^{4c} is a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

In one embodiment, L^{4c} is a divalent, branched or unbranched, saturated or unsaturated, hydrocarbon chain, having from 1 to 30 carbon atoms, wherein one or more of the carbon atoms is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more halo or oxo (═O).

In one embodiment, the group:

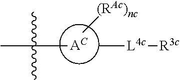

is selected from the group consisting of:

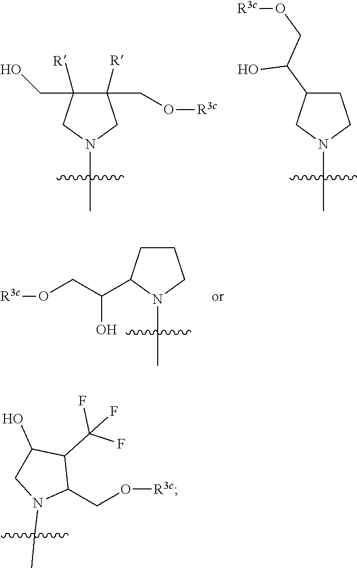

wherein

each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

In one embodiment, the group:

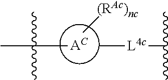

is selected from the group consisting of:

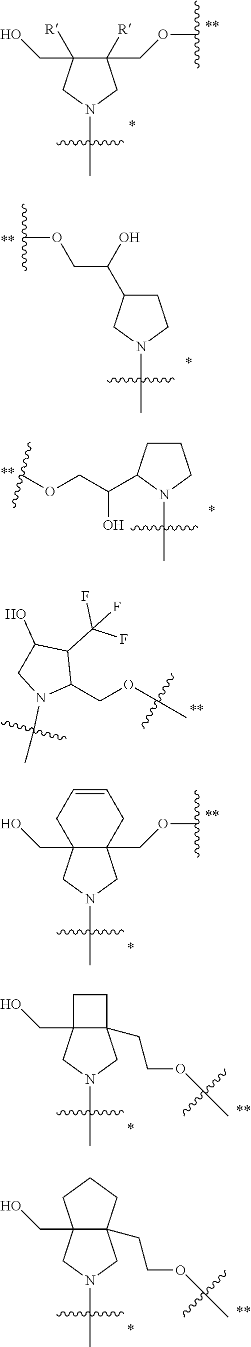

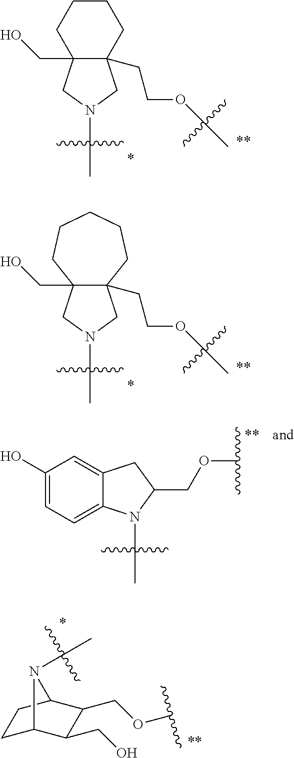

wherein:

each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;

the valence marked with * is attached to L^{3c}; and

the valence marked with ** is attached to R^{3c}.

In one embodiment, the group:

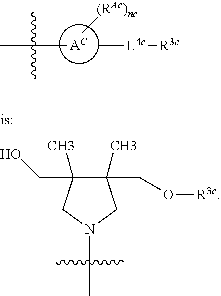

In one embodiment, L^{4c} is connected to R^{3c} through —O—.

In one embodiment, R^{3c} is attached to the reminder of the conjugate through the oxygen of a phosphate of the nucleic acid molecule.

In one embodiment, R^{3c} is attached to the reminder of the conjugate through the oxygen of a phosphate at the 5′-end of a sense or the antisense strand.

In one embodiment, R^{3c} is attached to the reminder of the conjugate through the oxygen of a phosphate at the 3′-end of a sense or the antisense strand.

In one embodiment, R^{3c} is attached to the reminder of the conjugate through the oxygen of a phosphate at the 3′-end of a sense strand.

In one embodiment, the compound of formula (I) is selected from the group consisting of:

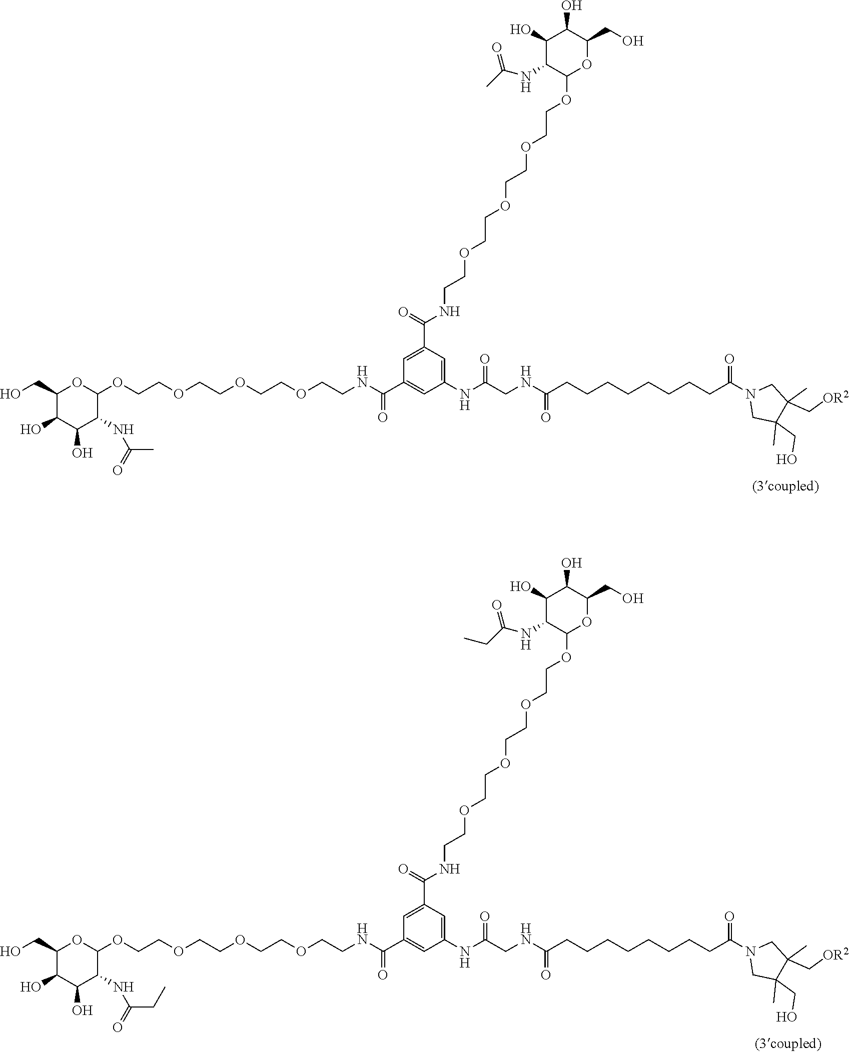

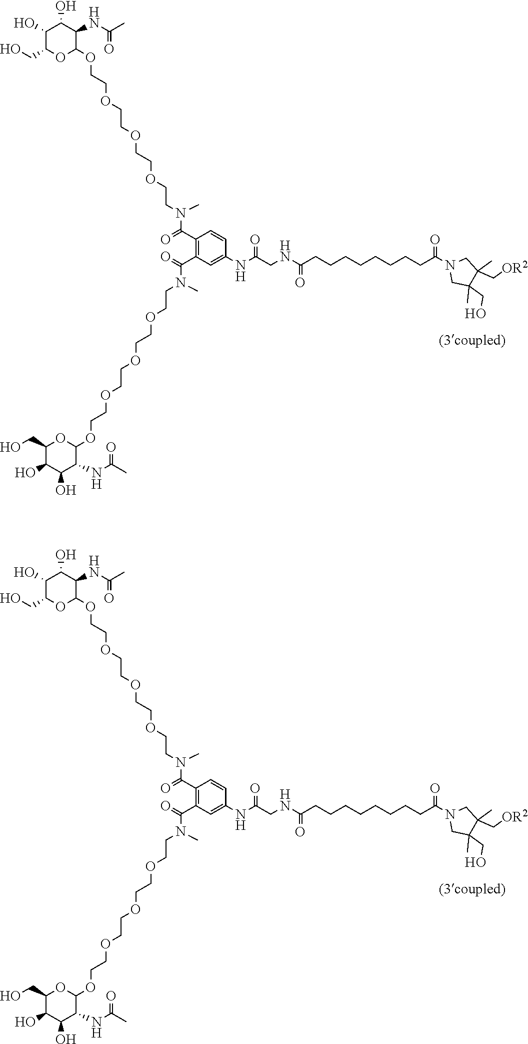

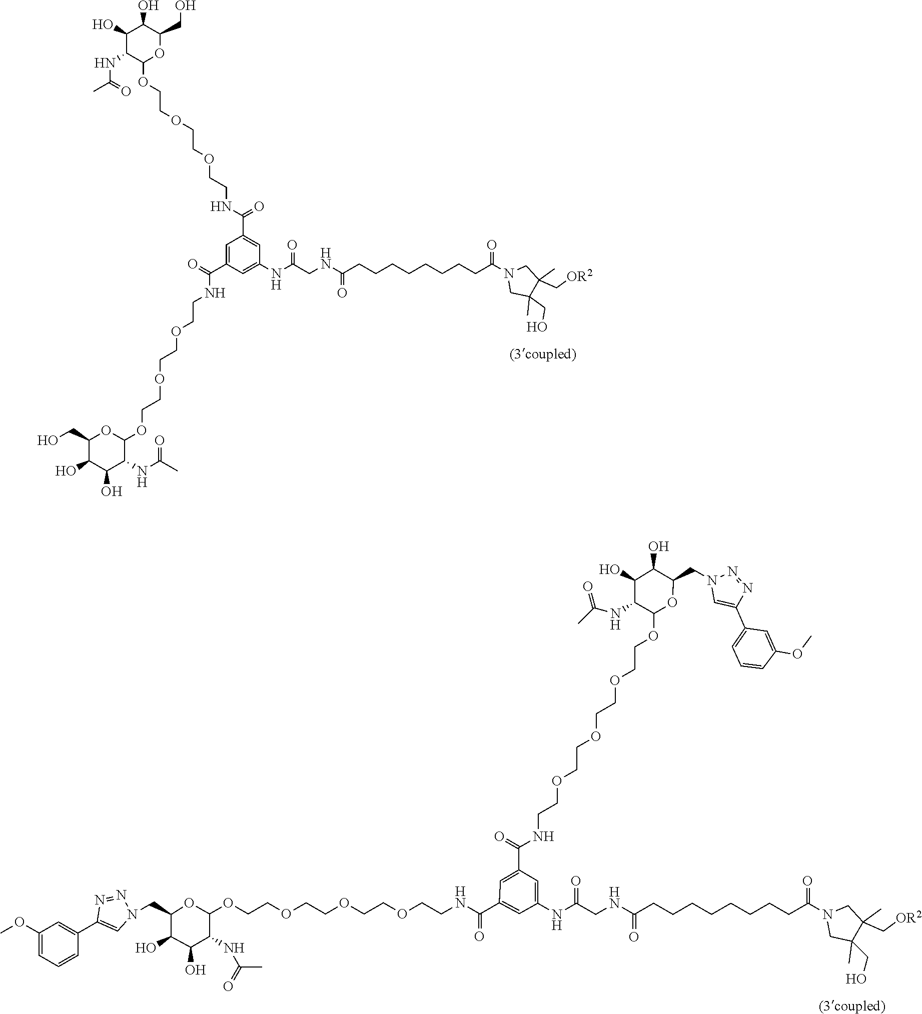

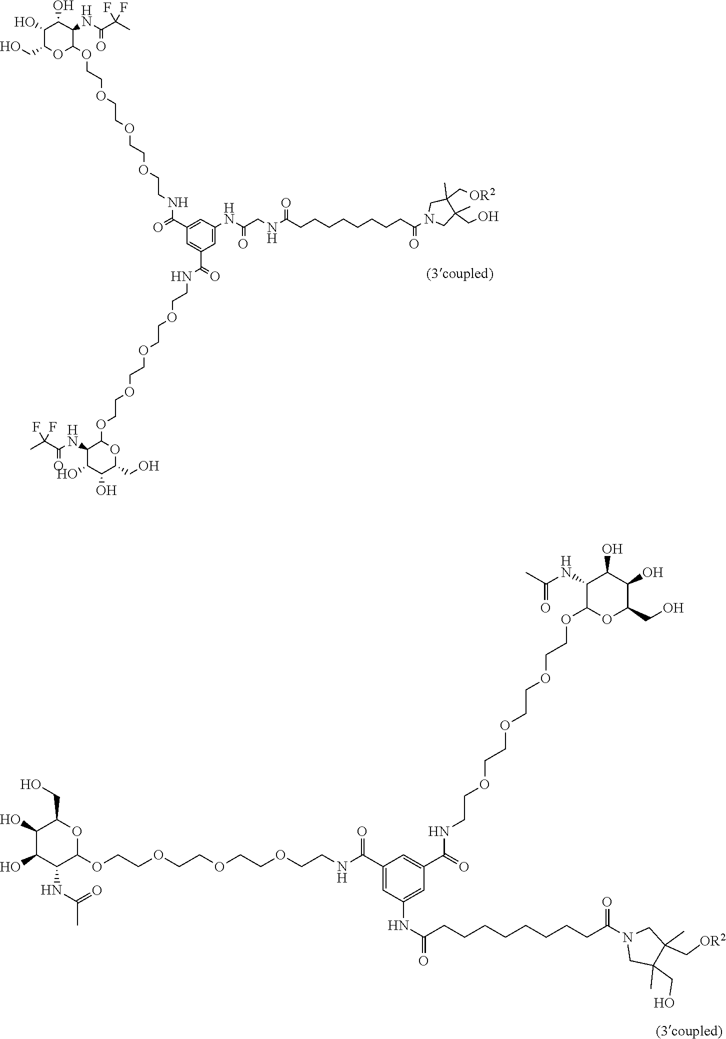

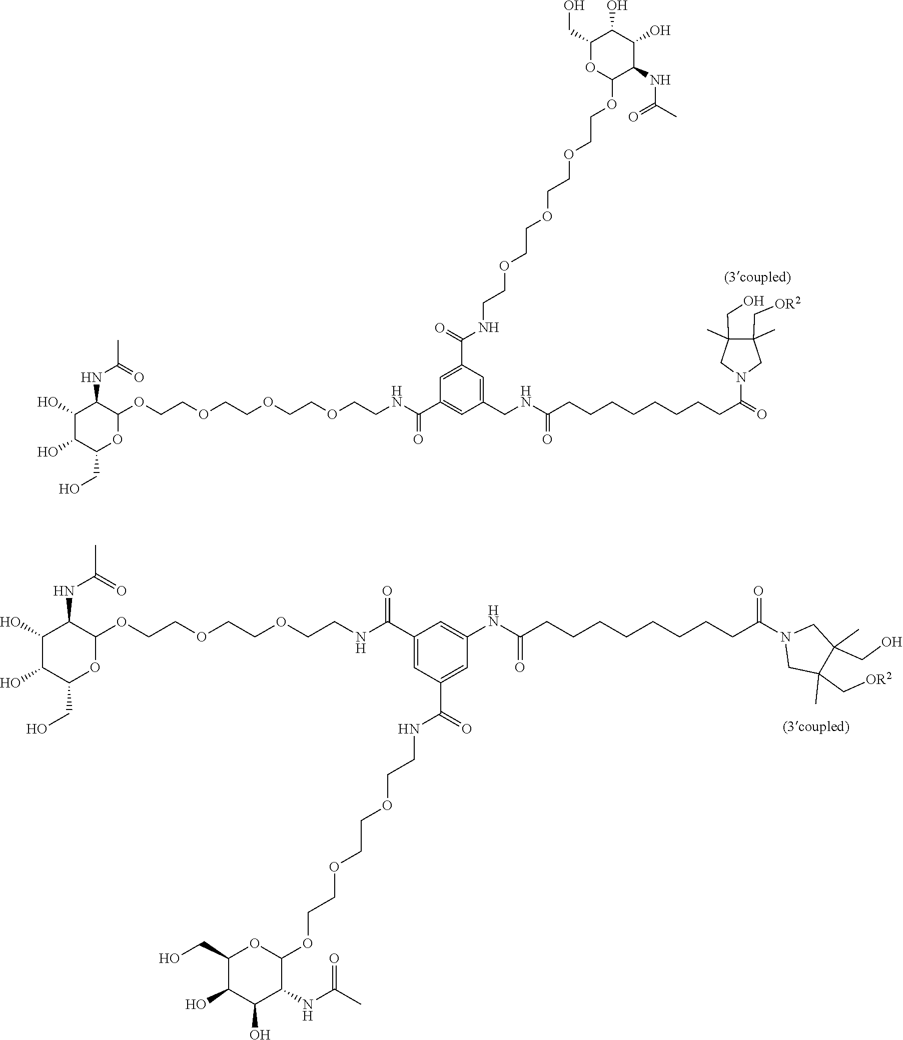

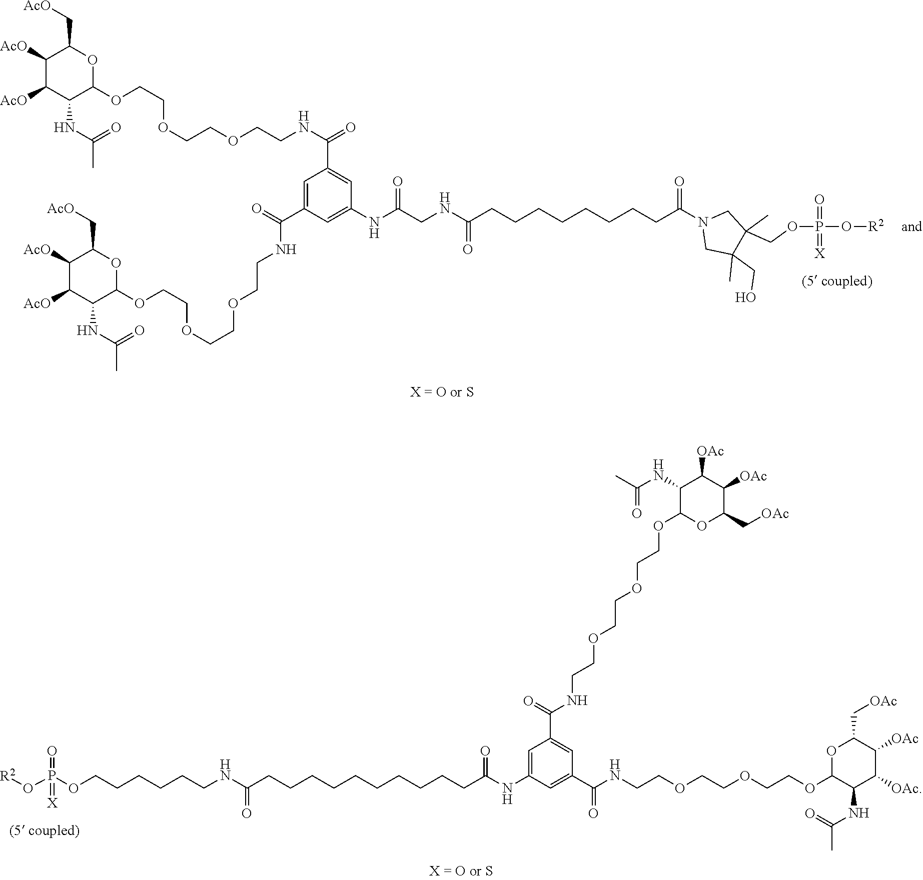

In one embodiment the targeted nucleic acid conjugate is a targeted nucleic acid conjugate as described in WO2015/006740, WO2016/028649, U.S. Pat. No. 8,106,022B2, U.S. Pat. No. 8,450,467B2, U.S. Pat. No. 8,828,956B2, WO2016/149020, WO2017/156012, WO2018/044350, WO2016/100401, WO2018/039364, WO2018/044350, WO2017/174657, WO2018/185210, WO2018/185252, WO2018/185253, U.S. Pat. No. 9,943,604B2, or U.S. Pat. No. 9,714,421B2 The present invention will be described in greater detail by way of specific examples.

The following examples are offered for illustrative purposes, and are not intended to limit the invention in any manner. Those of skill in the art will readily recognize a variety of noncritical parameters which can be changed or modified to yield essentially the same results.

### EXAMPLES

Membrane destabilizing polymers can be prepared using starting materials and synthetic methods that are similar to those described in International Patent Application Publication Numbers WO2015/017519 and WO2016/118697.

Targeted nucleic acid conjugates can be prepared as described in International Patent Application Publication Number WO2017/177326 and as described below.

### Example 1. Synthesis of Conjugate 1

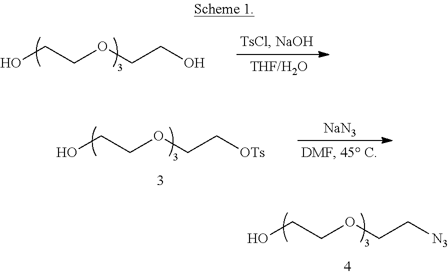

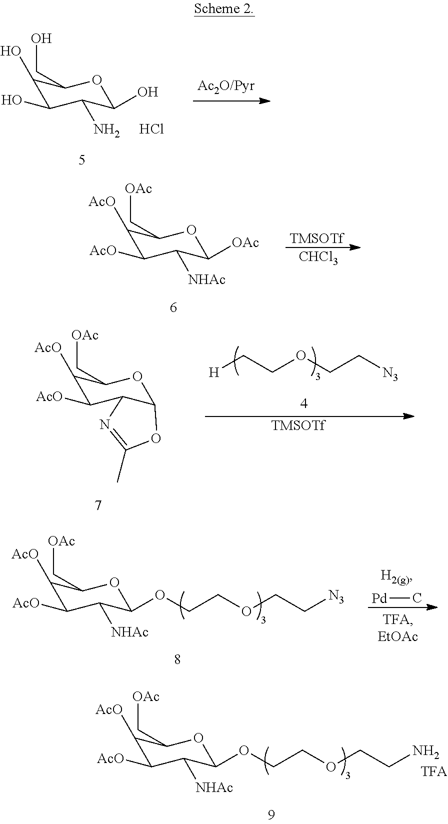

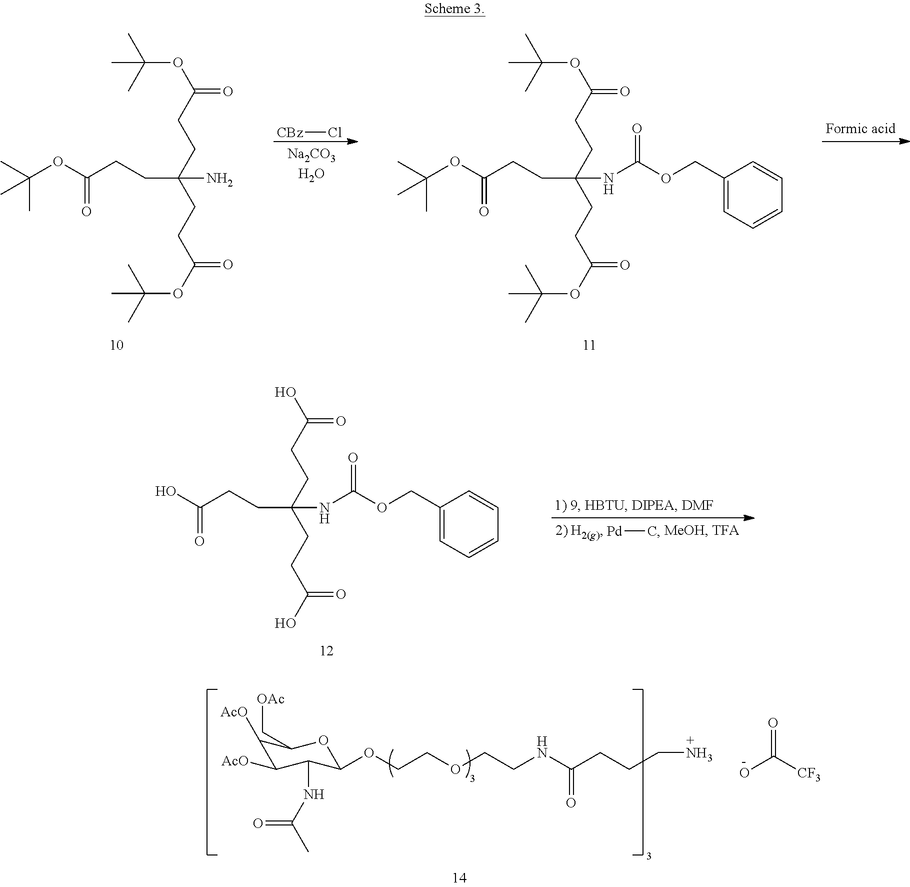

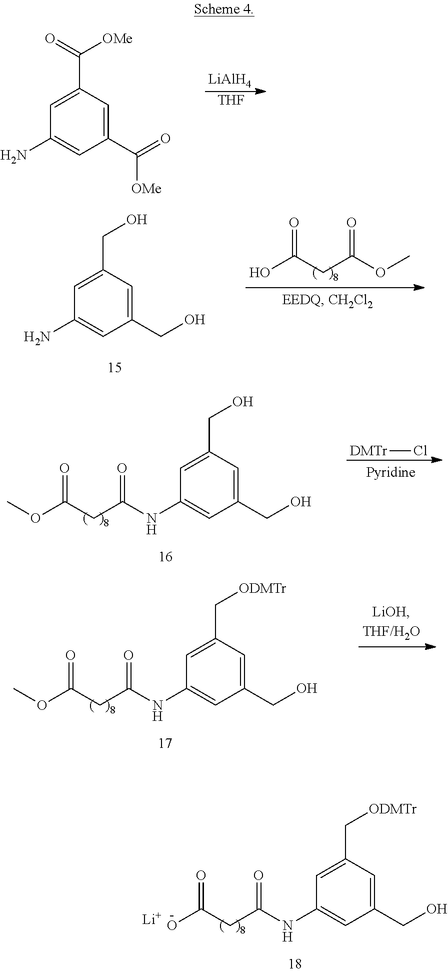

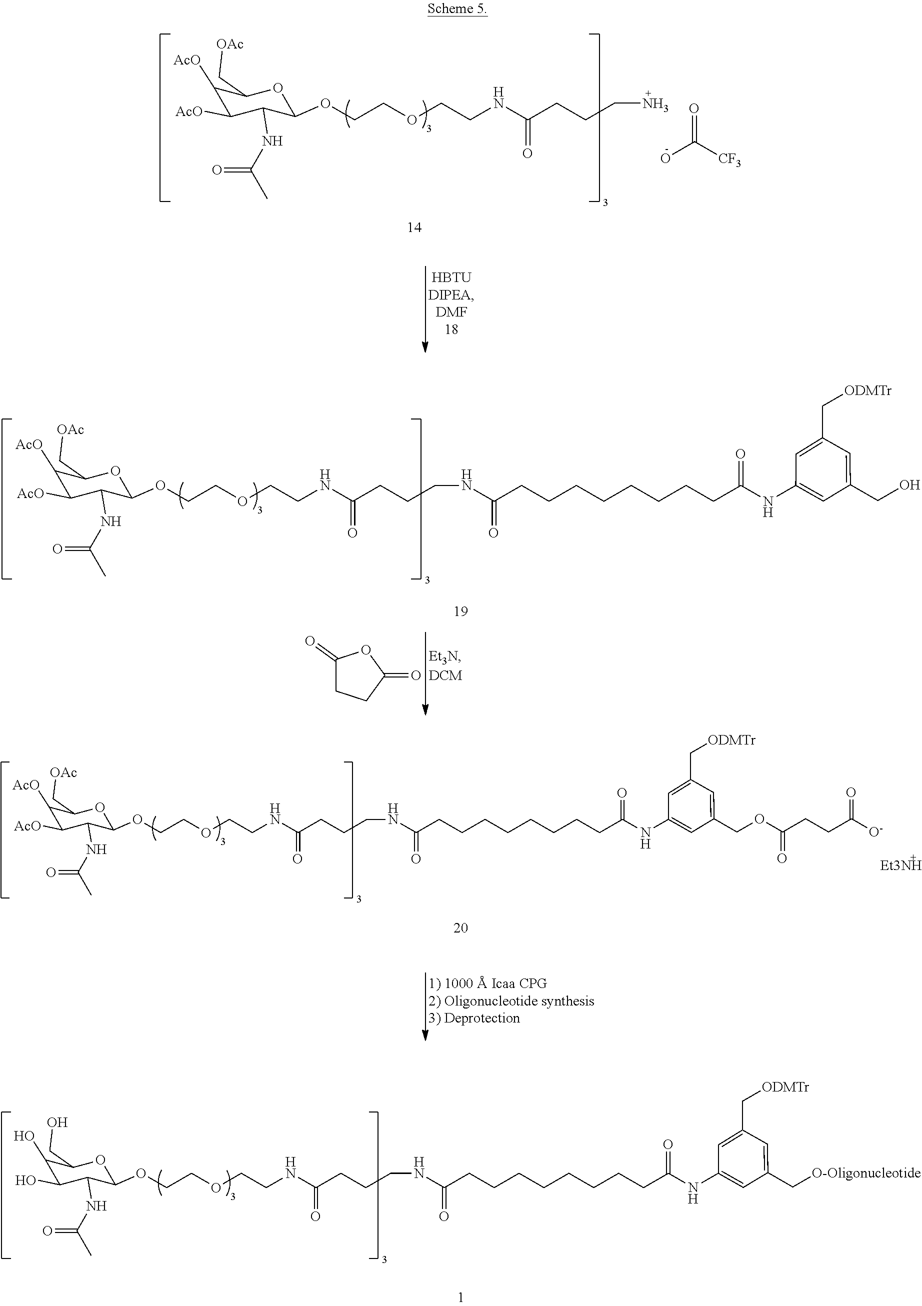

### Step 1. Preparation of 2-(2-(2-(2-hydroxyethoxy)ethoxy)ethoxy)ethyl 4-methylbenzenesulfonate 3

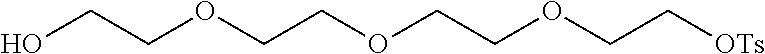 [SMILES: Cc1ccc(S(=O)(=O)OCCOCCOCCOCCO)cc1]

A solution of tetraethylene glycol (934 g, 4.8 mol) in THF (175 mL) and aqueous NaOH (5M, 145 mL) was cooled (0° C.) and treated with p-Toluensulfonyl chloride (91.4 g, 480 mmol) dissolved in THF (605 mL) and then stirred for two hours (0° C.). The reaction mixture was diluted with water (3 L) and extracted (3×500 mL) with CH₂Cl₂. The combined extracts were washed with water and brine then dried (MgSO₄), filtered and concentrated to afford 2-(2-(2-(2-hydroxyethoxy)ethoxy)ethoxy)ethyl 4-methylbenzenesulfonate 3 (140 g, 84%) as a pale yellow oil. Rr (0.57, 10% MeOH—CH₂Cl₂).

### Step 2. Preparation of 2-(2-(2-(2-azidoethoxy)ethoxy)ethoxy)ethan-1-ol 4

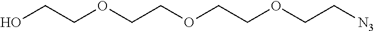 [SMILES: [N-]=[N+]=NCCOCCOCCOCCO]

A solution of 3 (140 g, 403 mmol) in DMF (880 mL) was treated with sodium azide (131 g, 2.02 mol) and heated (45° C.) overnight. A majority of the DMF was removed under reduced pressure and the residue was dissolved in CH₂Cl₂ (500 mL) and washed (3×500 mL) with brine then dried (MgSO₄), filtered and concentrated. The residue was passed through a short bed of silica (5% MeOH—CH₂Cl₂) and concentrated to yield 2-(2-(2-(2-azidoethoxy)ethoxy)ethoxy)ethan-1-ol 4 (65 g, 74%) as a yellow oil. Rr (0.56, 10% MeOH—CH₂Cl₂).

### Step 3. Preparation of Peracetylated Galactosamine 6

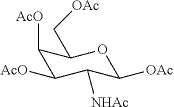 [SMILES: CC(=O)NC1C(OC(C)=O)OC(COC(C)=O)C(OC(C)=O)C1OC(C)=O]

D-Galactosamine hydrochloride 5 (250 g, 1.16 mol) in pyridine (1.5 L) was treated with acetic anhydride (1.25 L, 13.2 mol) over 45 minutes. After stirring overnight the reaction mixture was divided into three 1 L portions. Each 1 L portion was poured into 3 L of ice water and mixed for one hour. After mixing the solids were filtered off, combined, frozen over liquid nitrogen and then lyophilized for five days to yield peracetylated galactosamine 6 (369.4 g, 82%) as a white solid. Rf (0.58, 10% MeOH—CH₂Cl₂).

### Step 4. Preparation of (3aR,5R,6R,7R,7aR)-5-(acetoxymethyl)-2-methyl-3a,6,7,7a-tetrahydro-5H-pyrano[3,2-d]oxazole-6,7-diyl diacetate 7

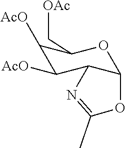 [SMILES: CC(=O)OCC1OC2OC(C)=NC2C(OC(C)=O)C1OC(C)=O]

A solution of per-acetylated galactosamine 6 (8.45 g, 21.7 mmol) in CHCl₃ (320 mL) was treated dropwise with TMSOTf (4.32 mL, 23.9 mmol). After stirring (1.5 hr, 40° C.) the reaction was quenched by the addition of triethylamine (5 mL) and concentrated to dryness to afford compound 7 as a pale yellow glass (7.2 g, Quant.). The product was used without further purification. Rf (0.59, 10% MeOH—CH₂Cl₂).

### Step 5. Preparation of (2R,3R,4R,5R,6R)-5-acetamido-2-(acetoxymethyl)-6-(2-(2-(2-(2-azidoethoxy)ethoxy)ethoxy)ethoxy)tetrahydro-2H-pyran-3,4-diyl diacetate 8

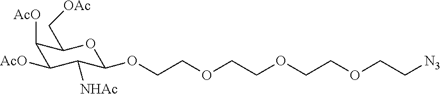 [SMILES: CC(=O)NC1C(OCCOCCOCCOCCN=[N+]=[N-])OC(COC(C)=O)C(OC(C)=O)C1OC(C)=O]

Compound 7 (7.2 g, 21.7 mmol) and 2-(2-(2-(2-azidoethoxy)ethoxy)ethoxy)ethan-1-ol 4 (2.65 g, 15.2 mmol) were azeotroped (3×) from toluene (150 mL) to remove traces of water. The dried material was dissolved in 1,2-dichloroethane (150 mL), cooled (˜5° C.) and treated with TMSOTf (784 μL, 4.34 mmol). After stirring overnight the reaction was quenched by the addition of triethylamine (5 mL) and concentrated. The residue was purified by chromatography (1%→5% MeOH—CH₂Cl₂) to afford 8 (7.12 g, 85%) as a brown oil. Rf (0.3, 10% MeOH—CH₂Cl₂).

### Step 6. Preparation of 2-(2-(2-(2-(((2R,3R,4R,5R,6R)-3-acetamido-4,5-diacetoxy-6-(acetoxymethyl)tetrahydro-2H-pyran-2-yl)oxy)ethoxy)ethoxy)ethoxy)ethan-1-aminium 2,2,2-trifluoroacetate 9

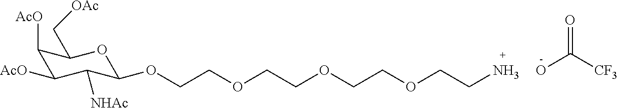

A solution of the azide 8 (7.12 g, 13 mmol) in EtOAc (150 mL) and trifluoroacetic acid (2 mL) was treated with palladium on charcoal (1.5 g, 10% w/w wet basis). The reaction mixture was then purged with hydrogen and stirred vigorously overnight. After purging with nitrogen, the mixture was filtered through Celite, rinsing with MeOH. The filtrate was concentrated and purified via chromatography (5%→10%→20% MeOH—CH₂Cl₂) to afford 9 (5.8 g, 72%) as a brown oil. Rf (0.34, 15% MeOH—CH₂Cl₂).

### Step 7. Preparation of di-tert-butyl 4-(((benzyloxy)carbonyl)amino)-4-(3-(tert-butoxy)-3-oxopropyl)heptanedioate 11

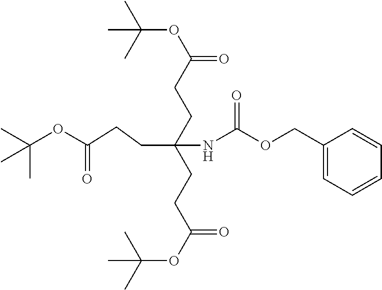 [SMILES: CC(C)(C)OC(=O)CCC(CCC(=O)OC(C)(C)C)(CCC(=O)OC(C)(C)C)NC(=O)OCc1ccccc1]

To a solution of di-tert-butyl 4-amino-4-(3-(tert-butoxy)-3-oxopropyl)heptanedioate 10 (13.5 g, 33 mmol), 25% Na₂CO_{3 (aq)} (150 mL) and dichloromethane (300 mL) was added slowly benzyl chloroformate (14 mL, 98 mmol). The solution was stirred vigorously overnight (16h) at room temperature. Upon completion, additional dichloromethane (100 mL) was added and the dichloromethane layer was separated. The aqueous layer was extracted with dichloromethane (2×100 mL). The combine dichloromethane extracts were dried on magnesium sulfate, filtered and concentrated to dryness. The product 11 was isolated as a colorless oil that required no further purification (15.8 g, 88%). Rf (0.7, 1:1 EtOAc-Hexane).

### Step 8. Preparation of 4-(((benzyloxy)carbonyl)amino)-4-(2-carboxyethyl)heptanedioic acid 12

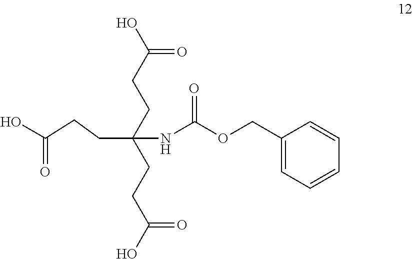 [SMILES: O=C(O)CCC(CCC(=O)O)(CCC(=O)O)NC(=O)OCc1ccccc1]

A solution of 11 (15.6 g, 28.8 mmol) in formic acid (50 mL) was stirred at room temperature for 2 hours. The solution was concentrated to dryness and dissolved in ethyl acetate (˜25 mL). Upon standing, the product crystallized as a colorless solid. The solid was filtered, washed with ethyl acetate and air dried to afford 12 as a colorless solid (10.2 g, 93%). Rf (0.1, 10% MeOH—CH₂Cl₂).

### Step 9. Preparation of Compound 13

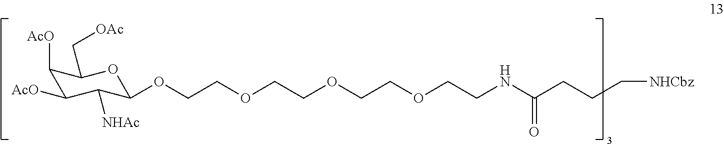

A solution of 12 (793 mg, 2.08 mmol) and 9 (5.8 g, 9.36 mmol) in DMF (50 mL) was treated with BOP (3.67 g, 8.32 mmol) then N,N-diisopropylethylamine (4.31 mL, 25 mmol). After stirring overnight the mixture was concentrated to dryness and subjected to chromatography (1%→2%→5%→10%→15% MeOH—CH₂Cl₂) to afford 13 (5.71 g [crude], >100%—contained coupling by-products that did not affect the next step). Rf (0.45, 10% MeOH—CH₂Cl₂).

### Step 10. Preparation of Compound 14

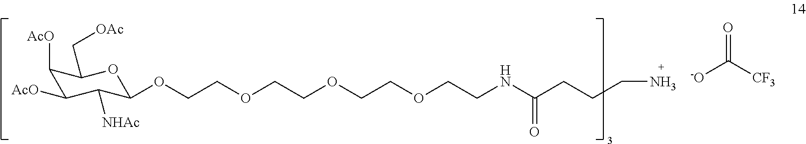

Compound 13 (5.7 g) was dissolved in MeOH (150 mL) and TFA (1.5 mL) and treated with palladium on charcoal (1 g, 10% w/w wet basis). The reaction mixture was then purged with hydrogen and stirred vigorously overnight. After purging with nitrogen, the mixture was filtered through Celite, rinsing with MeOH. The filtrate was concentrated and purified via chromatography (5%→10%→20% MeOH—CH₂Cl₂) to afford 14 as a brown oil (2.15 g, 56% over two steps). Rf (0.32, 10% MeOH—CH₂Cl₂).

### Step 11. Preparation of (5-amino-1,3-phenylene)dimethanol 15

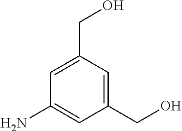 [SMILES: Nc1cc(CO)cc(CO)c1]

A solution of dimethyl 5-aminoisophthalate (20.0 g, 96 mmol) in THF (350 mL) was added, dropwise, to a refluxing mixture of 3.75 eq LiAlH₄ (13.6 g, 358 mmol) in THF (440 mL) over one hour. The mixture was stirred at reflux for a further two hours, then cooled to room temperature and quenched by the careful addition of MeOH (27 mL) then water (40 mL). After stirring the quenched mixture for two hours it was filtered and concentrated to dryness. The residue was recrystallized (2×) from EtOAc to afford 15 as brownish-yellow crystals (10.2 g, 70%).

### Step 12. Preparation of methyl 10-((3,5-bis(hydroxymethyl)phenyl)amino)-10-oxodecanoate 16

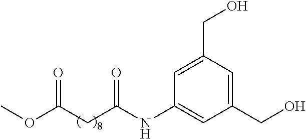

A solution of methyl sebacate (3.8 g, 17 mmol), 15 (2.5 g, 17 mmol) and EEDQ (8.1 g, 33 mmol) in 2:1 dichloromethane/methanol (200 mL) was stirred at room temperature for 2 hours. Upon completion the solution was concentrated to dryness. The solid obtained was triturated with dichloromethane (50 mL) and filtered. The solid was rinsed with cold dichloromethane and air dried to afford 16 as a colorless solid (4.3 g, 72%). Rf (0.33, EtOAc).

### Step 13. Preparation of methyl 10-((3-((bis(4-methoxyphenyl)(phenyl)methoxy)methyl)-5-(hydroxymethyl)phenyl)amino)-10-oxodecanoate 17

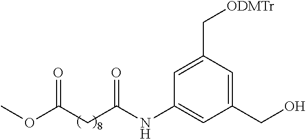

To a solution of 16 (4.3 g, 12 mmol) in pyridine (50 mL) was added 4,4′-(chloro(phenyl)methylene)bis(methoxybenzene) (4.1 g, 12 mmol). The solution was stirred under nitrogen overnight at room temperature. Upon completion the solution was concentrated to dryness and the residue was purified by column chromatography (0.5%→0.75%→1%→1.5% MeOH—CH₂Cl₂) to afford 17 as a yellow solid (2.9 g, 35%). Rf (0.6, 10% MeOH—CH₂Cl₂).

### Step 14. Preparation of lithium 10-((3-((bis(4-methoxyphenyl)(phenyl)methoxy)methyl)-5-(hydroxymethyl)phenyl)amino)-10-oxodecanoate 18

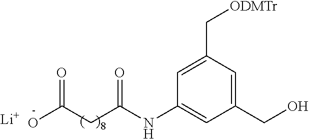

To a solution of 17 (2.9 g, 4.3 mmol) in THF (60 mL) was added water (15 mL) and lithium hydroxide (112 mg, 4.7 mmol). The solution was stirred overnight at room temperature. Upon completion the solution was concentrated to remove the THF. The remaining aqueous solution was flash frozen on liquid nitrogen and lyophilized overnight to afford a colorless solid (2.9 g, quant.). Rf (0.3, 10% MeOH—CH₂Cl₂).

### Step 15. Preparation of Compound 19

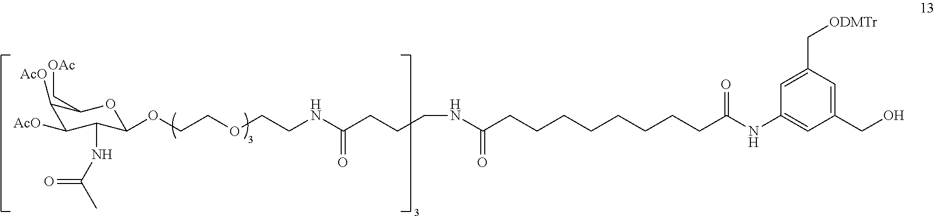

To a solution 14 (454 mg, 0.67 mmol), 18 (1.25 g, 0.67 mmol) and HBTU (381 mg, 1.0 mmol) in anhydrous DMF (25 mL) was added N,N-diisopropylethylamine (0.35 mL, 2.0 mmol). The solution was stirred overnight at room temperature. Upon completion, the solution was poured into ethyl acetate (250 mL) and washed with brine (3×200 mL). The ethyl acetate layer was dried on magnesium sulfate, filtered and concentration to dryness. Purification by column chromatography (5%→7.5%→10%→15% MeOH in CH₂Cl₂) afforded 19 as a pale orange foam (1.5 g, 94%). Rf (0.25, 10% MeOH—CH₂Cl₂).

### Step 16. Preparation of Compound 20

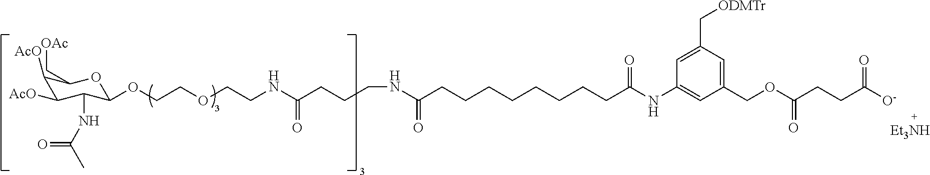

A solution of compound 19 (1.5 g, 0.6 mmol), succinic anhydride (120 mg, 1.2 mmol), DMAP (220 mg, 1.8 mmol) and trimethylamine (250 μL, 1.8 mmol) in anhydrous CH₂Cl₂ (50 mL) was stirred overnight at room temperature. Upon completion, the solution was concentrated to dryness and filtered through a short plug of silica (100% CH₂Cl₂→15% MeOH in CH₂Cl₂) to afford the product 20 as a light beige foam (1.1 g, 70%). Mass m/z (ES-TOF MS) 727.7 [M+3H-DMTr]⁺, 1091.1 [M+2H-DMTr]. ¹H NMR (400 MHz, CDCl₃) δ 8.92 (br s, 1H), 7.78 (s, 1H), 7.49-7.47 (m, 3H), 7.41 (br s, 1H), 7.38-7.34 (m, 5H), 7.32-7.26 (m, 4H), 7.24-7.08 (br s, 3H), 7.08 (s, 1H), 6.90-6.80 (m, 7H), 5.31 (d, 3H, J=2.7 Hz), 5.12 (s, 2H), 5.06 (dd, 3H, J=11.2, 3.2 Hz), 4.78 (d, 3H, J=8.5 Hz), 4.24-4.08 (m, 12H), 3.95-3.88 (m, 7H), 3.85-3.76 (m, 4H), 3.78 (s, 6H), 3.68-3.56 (m, 34H), 3.54-3.44 (m, 8H), 3.41-3.33 (m, 6H), 2.70-2.60 (m, 4H), 2.52-2.30 (m, 30H), 2.24-2.16 (m, 8H), 2.14 (s, 9H), 2.04 (s, 9H), 2.02-1.96 (m, 6H), 1.98 (s, 9H), 1.96 (s, 9H), 1.74-1.52 (m, 4H), 1.36-1.24 (m, 12H).

### Step 17. Preparation of Conjugate 1

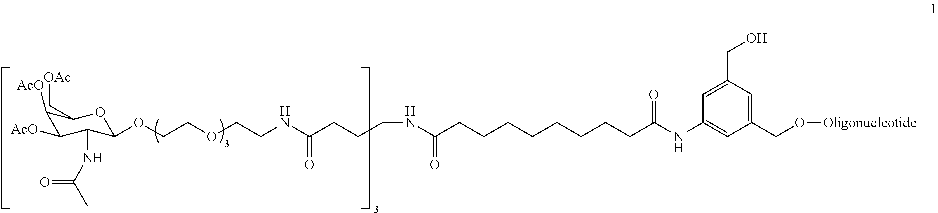

The succinate 20 was loaded onto 1000 Å LCAA (long chain aminoalkyl) CPG (control pore glass) using standard amide coupling chemistry. A solution of diisopropylcarbodiimide (52.6 μmol), N-hydroxy succinimide (0.3 mg, 2.6 μmol) and pyridine (10 μL) in anhydrous acetonitrile (0.3 mL) was added to 20 (20.6 mg, 8 μmol) in anhydrous dichloromethane (0.2 mL). This mixture was added to LCAA CPG (183 mg). The suspension was gently mixed overnight at room temperature. Upon disappearance of 20 (HPLC), the reaction mixture was filtered and the CPG was washed with 1 mL of each dichloromethane, acetonitrile, a solution of 5% acetic anhydride/5% N-methylimidazole/5% pyridine in THF, then THF, acetonitrile and dichloromethane. The CPG was then dried overnight under high vacuum. Loading was determined by standard DMTr assay by UV/Vis (504 nm) to be 25 μmol/g. The resulting GalNAc loaded CPG solid support was employed in automated oligonucleotide synthesis using standard procedures. Nucleotide deprotection followed by removal from the solid support (with concurrent galactosamine acetate deprotection) afforded the GalNAc-oligonucleotide conjugate 1 as a representative example.

### Example 2: Synthesis of Conjugate 34

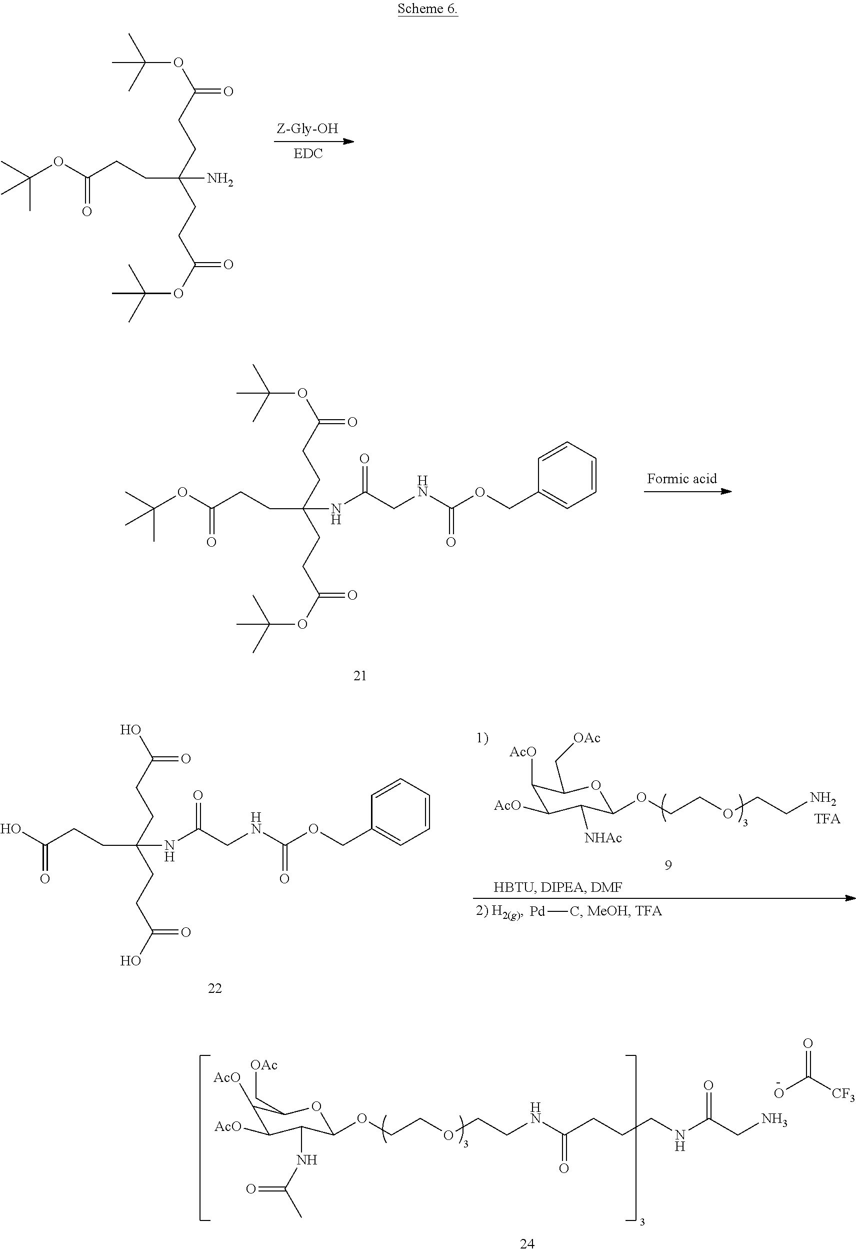

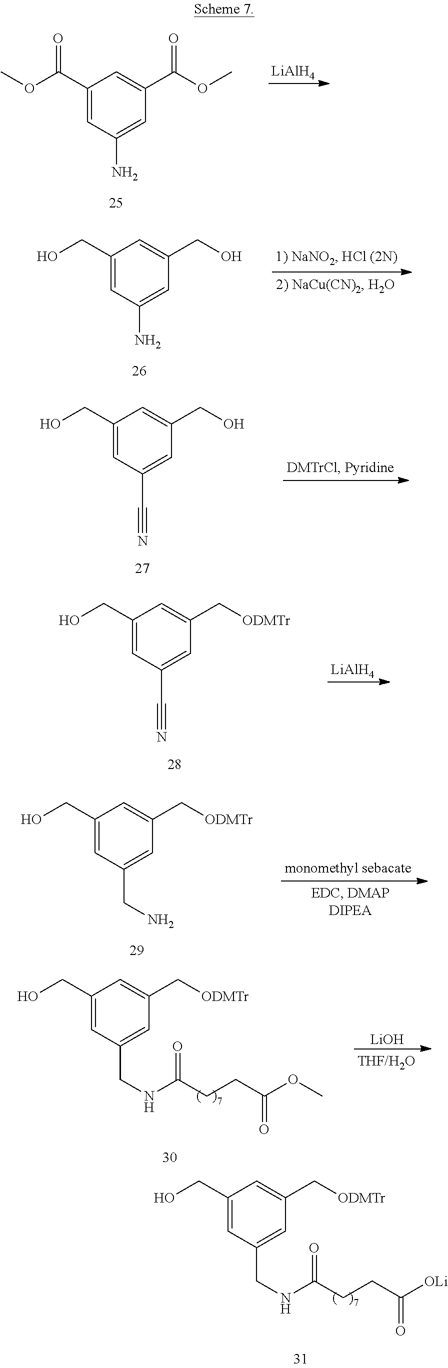

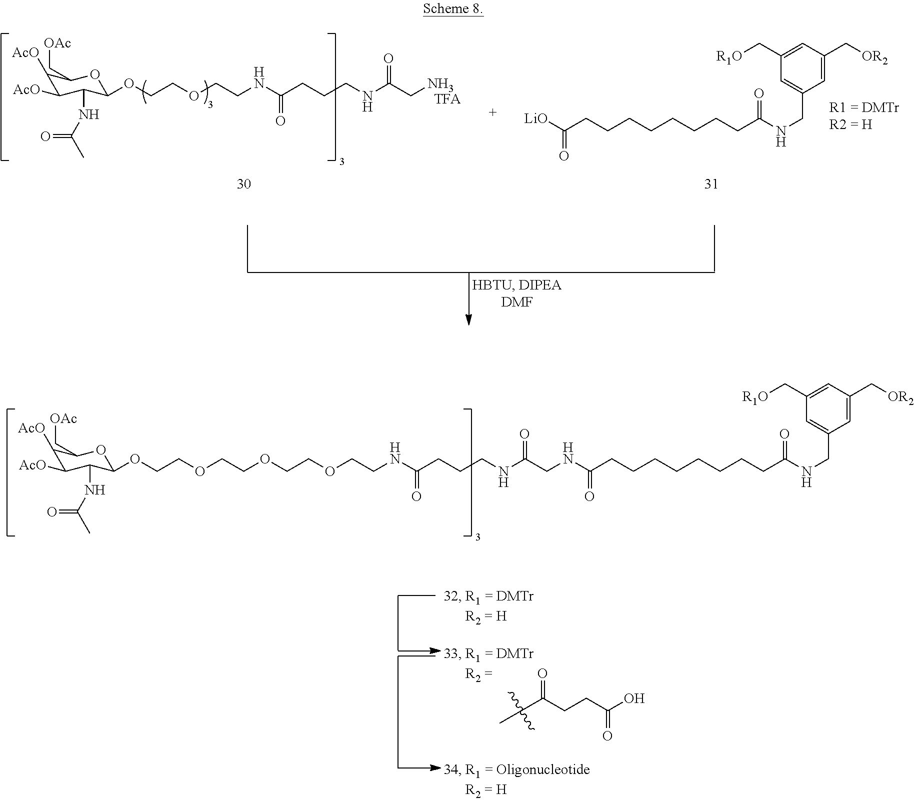

### Step 1. Preparation of di-tert-butyl 4-(2-(((benzyloxy)carbonyl)amino)acetamido)-4-(3-(tert-butoxy)-3-oxopropyl)heptanedioate 21

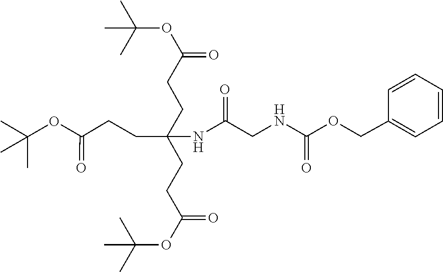 [SMILES: CC(C)(C)OC(=O)CCC(CCC(=O)OC(C)(C)C)(CCC(=O)OC(C)(C)C)NC(=O)CNC(=O)OCc1ccccc1]

A solution of di-tert-butyl 4-amino-4-(3-(tert-butoxy)-3-oxopropyl)heptanedioate (25 g, 60 mmol) and Z-glycine (18.9 g, 90.2 mmol) in CH₂Cl₂ (300 mL) was treated successively with EDC (23 g, 120 mmol), Diisopropylethylamine (32 mL, 180 mmol) and DMAP (Cat. 17 mg). After stirring (16h) the reaction mixture was poured into NaHCO₃ (Sat. Aq.), extracted with CH₂Cl₂, washed with brine, dried (MgSO₄), filtered and concentrated to afford di-tert-butyl 4-(2-(((benzyloxy)carbonyl)amino)acetamido)-4-(3-(tert-butoxy)-3-oxopropyl)heptanedioate 21 as an amorphous solid and was used without further processing (36 g, quant.). Rf (0.85, 10% MeOH—CH₂Cl₂).

### Step 2. Preparation of 4-(2-(((benzyloxy)carbonyl)amino)acetamido)-4-(2-carboxyethyl)heptanedioic acid 22

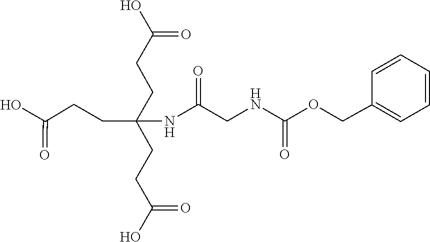 [SMILES: O=C(O)CCC(CCC(=O)O)(CCC(=O)O)NC(=O)CNC(=O)OCc1ccccc1]

A solution of di-tert-butyl 4-(2-(((benzyloxy)carbonyl)amino)acetamido)-4-(3-(tert-butoxy)-3-oxopropyl)heptanedioate 21 (59.3 mmol, 36 g) was stirred in neat formic acid (150 mL) for 72 hours. Upon completion, the formic acid was removed under reduced pressure and the crude solid was dried overnight on high-vacuum to yield 22 as a colorless solid (15.9 g, 61%). Rf (0.15, 10% MeOH—CH₂Cl₂).

### Step 3. Preparation of Compound 23

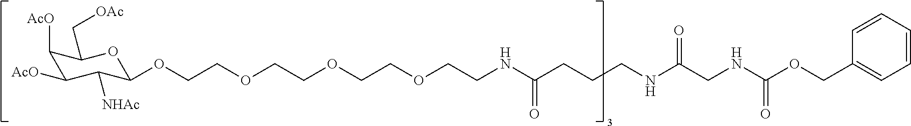

A solution of 22 (6.2 g, 14.1 mmol) and 2-(2-(2-(2-(((2R,3R,4R,5R,6R)-3-acetamido-4,5-diacetoxy-6-(acetoxymethyl)tetrahydro-2H-pyran-2-yl)oxy)ethoxy)ethoxy)ethoxy)ethan-1-aminium 2,2,2-trifluoroacetate (35 g, 56.5 mmol) in DMF (250 mL) was treated with BOP (25 g, 56.5 mmol) then N,N-diisopropylethylamine (29 mL, 170 mmol). After stirring overnight the mixture was concentrated to dryness and subjected to chromatography (100% CH₂Cl₂ to 15% MeOH—CH₂Cl₂) to afford compound 23 (24.6 g, 89%). Rf (0.55, 15% MeOH—CH₂Cl₂).

### Step 4. Preparation of Compound 24

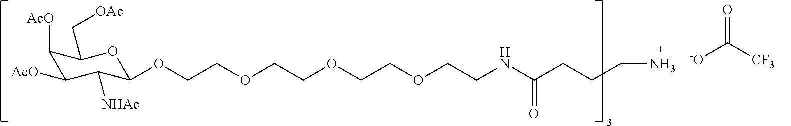

Compound 23 (24.6 g) was dissolved in MeOH (200 mL) and TFA (1.5 mL) and purged with nitrogen. Palladium on charcoal (1 g, 10% w/w wet basis) was added and then the reaction mixture was purged with hydrogen and stirred vigorously overnight. Upon completion, the reaction was purged with nitrogen, filtered through Celite and rinsed with MeOH. The filtrate was concentrated and purified by column chromatography on silica gel 60 (gradient: 5%→10%→20% MeOH—CH₂Cl₂) to afford 24 as a pale brown viscous oil (23 g). Rf (0.32, 10% MeOH—CH₂Cl₂).

### Step 5. Preparation of (5-amino-1,3-phenylene)dimethanol 26

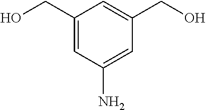 [SMILES: Nc1cc(CO)cc(CO)c1]

A suspension of lithium aluminum hydride (13.6 g, 358 mmol) in anhydrous tetrahydrofuran (450 mL) was brought to reflux under a nitrogen atmosphere and treated, dropwise, with a solution of dimethyl-5-aminoisophthalte 25 (20 g, 96 mmol) in anhydrous tetrahydrofuran (350 mL). After the addition was complete the mixture was heated to reflux for an additional 2 hours. Upon completion, the solution was cooled to room temperature and quenched by the slow addition of MeOH (27 mL) then water (40 mL). After stirring for 2 hours the mixture was filtered, concentrated and recrystallized from EtOAc to yield (5-amino-1,3-phenylene)dimethanol 26 as off-white crystals (10.2 g, 70%). Rf 0.5 (15% MeOH—CH₂Cl₂).

### Step 6. Preparation of 3,5-bis(hydroxymethyl)benzonitrile 27

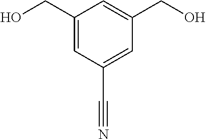 [SMILES: N#Cc1cc(CO)cc(CO)c1]

A solution of 26 (5 g, 33 mmol) in 2N hydrochloric acid (100 mL) was cooled to 0° C. and treated with a cold solution of sodium nitrite (3.53 g, 36 mmol) in water (50 mL). The reaction mixture was maintained at a temperature 5 5° C. for 30 min then treated with a solution of copper(I) cyanide (3.19 g, 35.6 mmol) and sodium cyanide (3.53 g, 72 mmol) in water (50 mL) in a single portion. After stirring overnight at room temperature the mixture was filtered, extracted with dichloromethane (3×100 mL), concentrated and used without further purification. The diol, 3,5-bis(hydroxymethyl)benzonitrile 27 was obtained as a yellow solid (2.19 g, 41%). Rf 0.75 (15% MeOH—CH₂Cl₂).

### Step 7. Preparation of 3-((bis(4-methoxyphenyl)(phenyl)methoxy)methyl)-5-(hydroxymethyl)benzonitrile 28

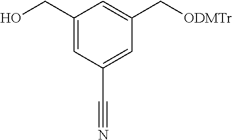

A solution of 3,5-bis(hydroxymethyl)benzonitrile 27 (538 mg, 3.3 mmol) in pyridine (14 mL) was treated with 4,4′-Dimethoxytrityl chloride (1.17 g, 3.46 mmol) and stirred overnight at room temperature. Once complete, the mixture was concentrated and dispersed in diethyl ether (25 mL), filtered and concentrated. The crude product was purified by column chromatography of silica gel 60 (gradient: 10% to 50% EtOAc-Hexane) to yield the 28 as a yellow solid (725 mg, 47%). Rf 0.5 (1:1 EtOAc-hexane).

### Step 8. Preparation of (3-(aminomethyl)-5-((bis(4-methoxyphenyl)(phenyl)methoxy)methyl)phenyl)methanol 29

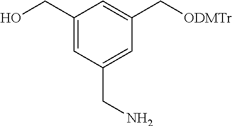

A solution of the 28 (100 mg, 0.22 mmol) in methyl tetrahydrofuran (5 mL) was cooled to 0° C. and treated slowly with lithium aluminum hydride (0.64 mmol=0.28 mL of a 2.3M solution in MeTHF). After stirring for one hour the reaction was quenched by the addition of methanol (1 mL) then water (0.3 mL) and stirred for 30 min. The mixture was filtered and concentrated, to yield (3-(aminomethyl)-5-((bis(4-methoxyphenyl)(phenyl)methoxy)methyl)phenyl)methanol 29 (78 mg, 77%). Rf 0.15 (10% MeOH—CH₂Cl₂).

### Step 9. Preparation of methyl 10-((3-((bis(4-methoxyphenyl)(phenyl)methoxy)methyl)-5-(hydroxymethyl)benzyl)amino)-10-oxodecanoate 30

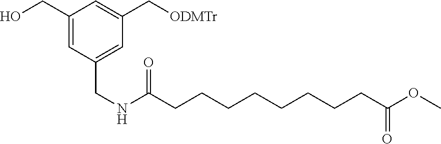

A solution of (3-(aminomethyl)-5-((bis(4-methoxyphenyl)(phenyl)methoxy)-methyl)phenyl)methanol 29 (78 mg, 0.17 mmol) and monomethyl sebacate (38 mg, 0.17 mmol) in dichloromethane (5 mL) were treated successively with EDC (48 mg, 0.25 mmol), DMAP (cat., 5 mg) and diisopropylethylamine (57 μL, 0.33 mmol). After stirring (3.5 hr) the reaction mixture was poured into saturated sodium bicarbonate solution (50 mL). The sodium bicarbonate solution was extracted with dichloromethane (3×50 mL), washed with brine (50 mL), dried on magnesium sulfate, filtered and concentrated to dryness. The crude material was purified by column chromatography on silica gel 60 (gradient: 2% to 5% MeOH—CH₂Cl₂) to afford methyl 10-((3-((bis(4-methoxyphenyl)(phenyl)methoxy)methyl)-5-(hydroxymethyl)benzyl)amino)-10-oxodecanoate 30 as a yellow oil (57 mg, 53%). Rf 0.45 (10% MeOH—CH₂Cl₂).

### Step 10. Preparation of lithium 10-((3-((bis(4-methoxyphenyl)(phenyl)methoxy)methyl)-5-(hydroxymethyl)benzyl)amino)-10-oxodecanoate 31

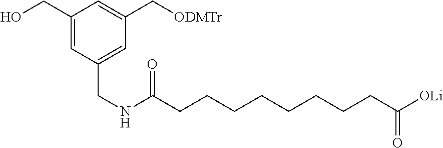

Compound 30 (188 mg, 0.28 mmol) was dissolved in tetrahydrofuran (5 mL) and treated with a solution of LiOH (7 mg, 0.30 mmol) in water (1 mL). Upon completion, the tetrahydrofuran was removed in vacuo and the remaining aqueous mixture was frozen and lyophilized to afford lithium 10-((3-((bis(4-methoxyphenyl)(phenyl)methoxy)methyl)-5-(hydroxymethyl)benzyl)amino)-10-oxodecanoate 31 as a colorless solid (180 mg, 99%). Rf 0.45 (10% MeOH—CH₂Cl₂).

### Step 11. Preparation of Compounds 32, 33, and 34

Compounds 32, 33 and 34 were prepared according to same procedure used to synthesize compounds 19, 20, and 1 respectfully.

### Example 3. Synthesis of Conjugate 36

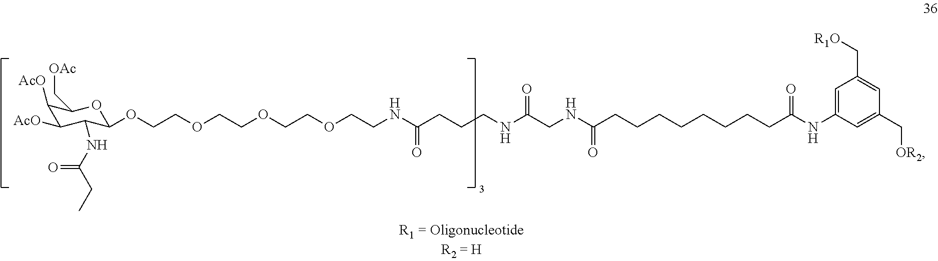

### Step 1. Preparation of Conjugate 36

Conjugate 36 was prepared using identical procedures as used to synthesize compound 34 and all corresponding intermediates. The only exception being the synthesis of compound 6 where propanoic anhydride was used in place of acetic anhydride.

### Example 4. Synthesis of Conjugate 42

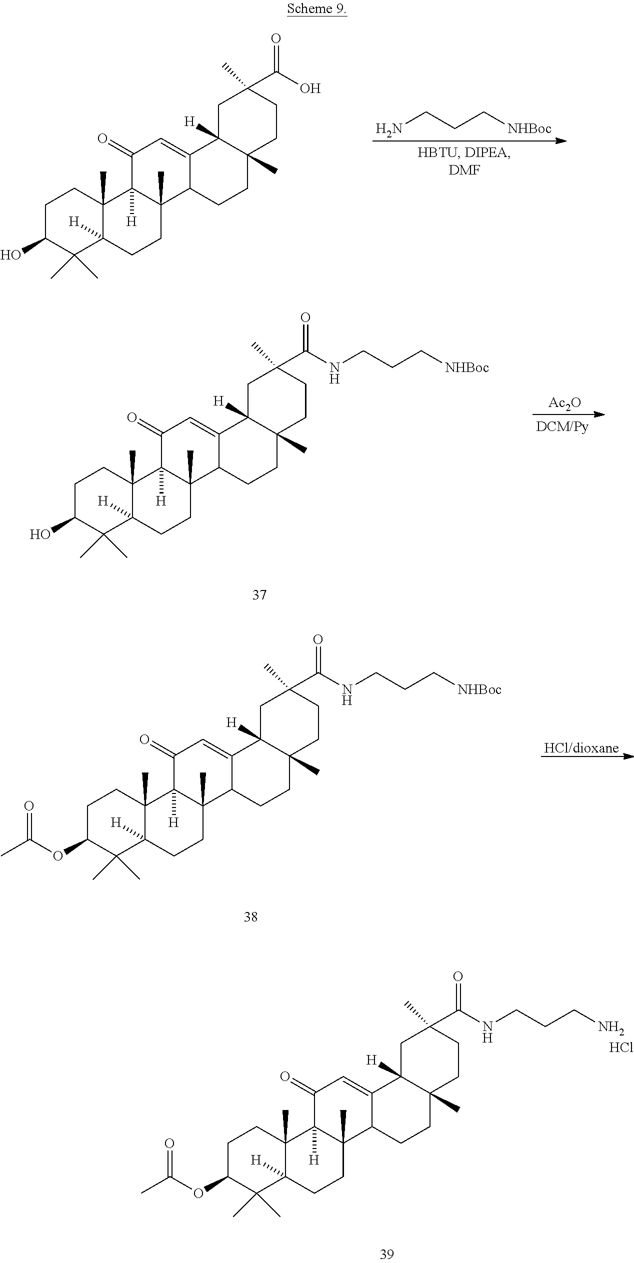

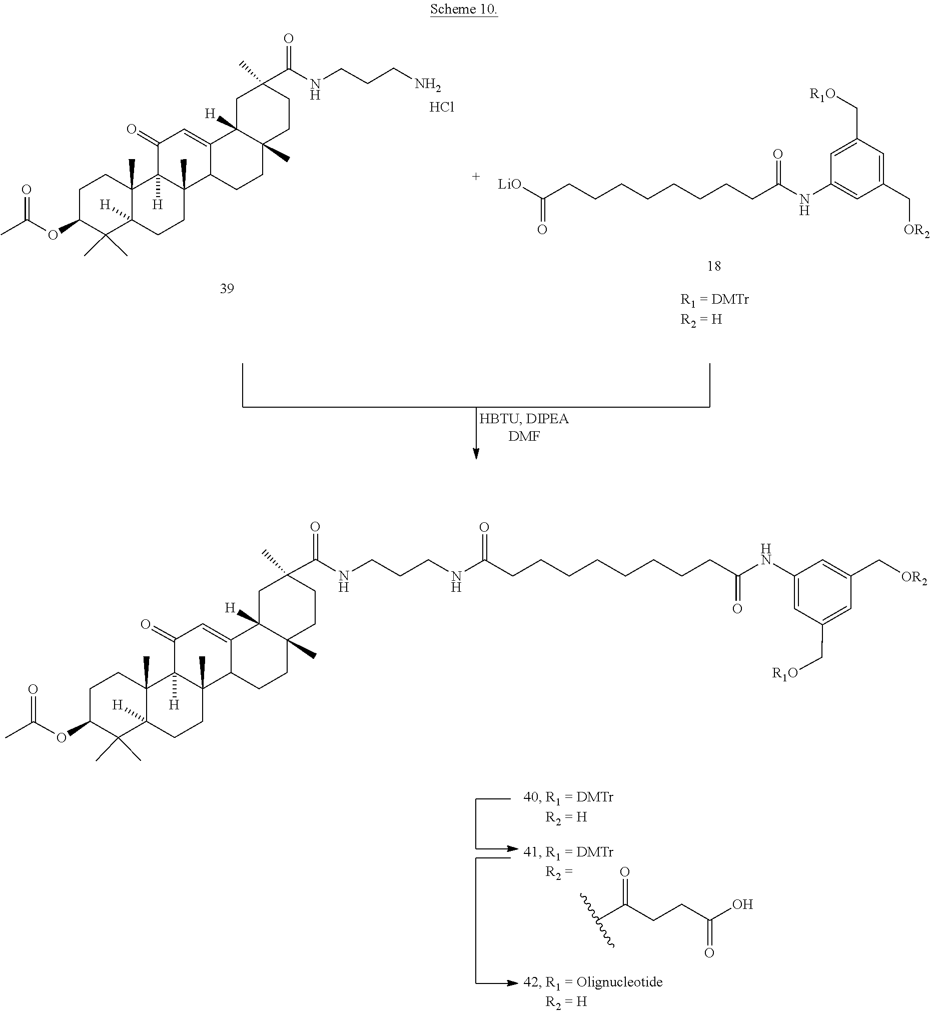

### Step 1. Preparation of Compound 37

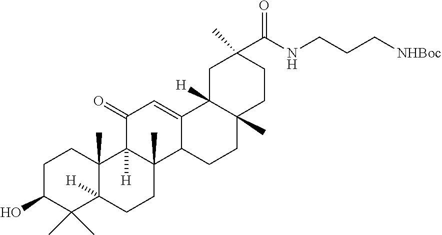

A solution of 18p-glycyrrhetinic acid (2.5 g, 5.3 mmol), tert-butyl (3-aminopropyl)carbamate (1.1 g, 6.4 mmol) and HBTU (3.0 g, 8.0 mmol) in N,N-dimethylformamide (20 mL) was added diisopropylethylamine (2.75 mL, 15.9 mmol). The solution was stirred overnight at room temperature. Upon completion, the solution was concentrated in vacuo to dryness. The residue was purified by column chromatography on silica gel 60 (gradient: 2% to 5% MeOH/CH₂Cl₂) to afford the product as a colorless solid (2.1 g, 63%).

### Step 2. Preparation of Compound 38

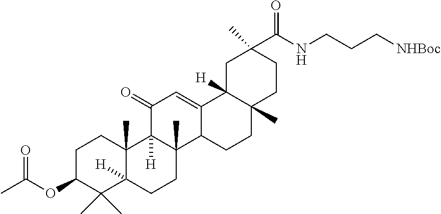

To a solution of 37 (2.1 g, 3.3 mmol) and triethylamine (3.5 mL, 10 mmol) in dichloromethane (25 mL) was added acetic anhydride (850 μL, 5.3 mmol) and DMAP (5 mg). The solution was stirred overnight at room temperature. Upon completion, the solution was concentrated to dryness and dissolved in ethyl acetate (100 mL), washed with water (100 mL), dried on magnesium sulfate, filtered and concentrated to dryness to afford a pale brown foam (1.9 g, 85%).

### Step 3. Preparation of Compound 39

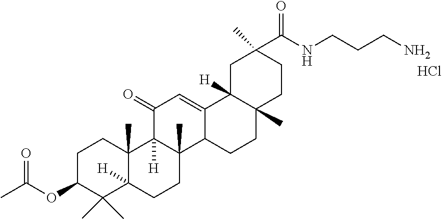

To a solution of 38 (1.5 g, 2.3 mmol) in anhydrous dioxane (25 mL) was added 2M Hydrogen chloride in dioxane (25 mL). The solution was stirred overnight at room temperature then concentrated in vacuo to dryness to afford a light brown solid (1.3 g, 96%).

### Step 4. Preparation of Compounds 40, 41 and 42

Compounds 40, 41 and 42 were prepared according to the same procedure used to synthesize compounds 19, 20, and 1 respectfully.

### Example 5. Synthesis of Conjugate 43

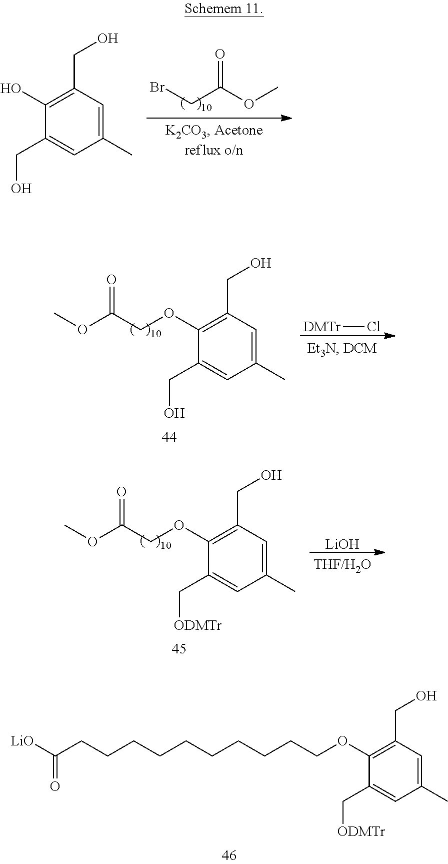

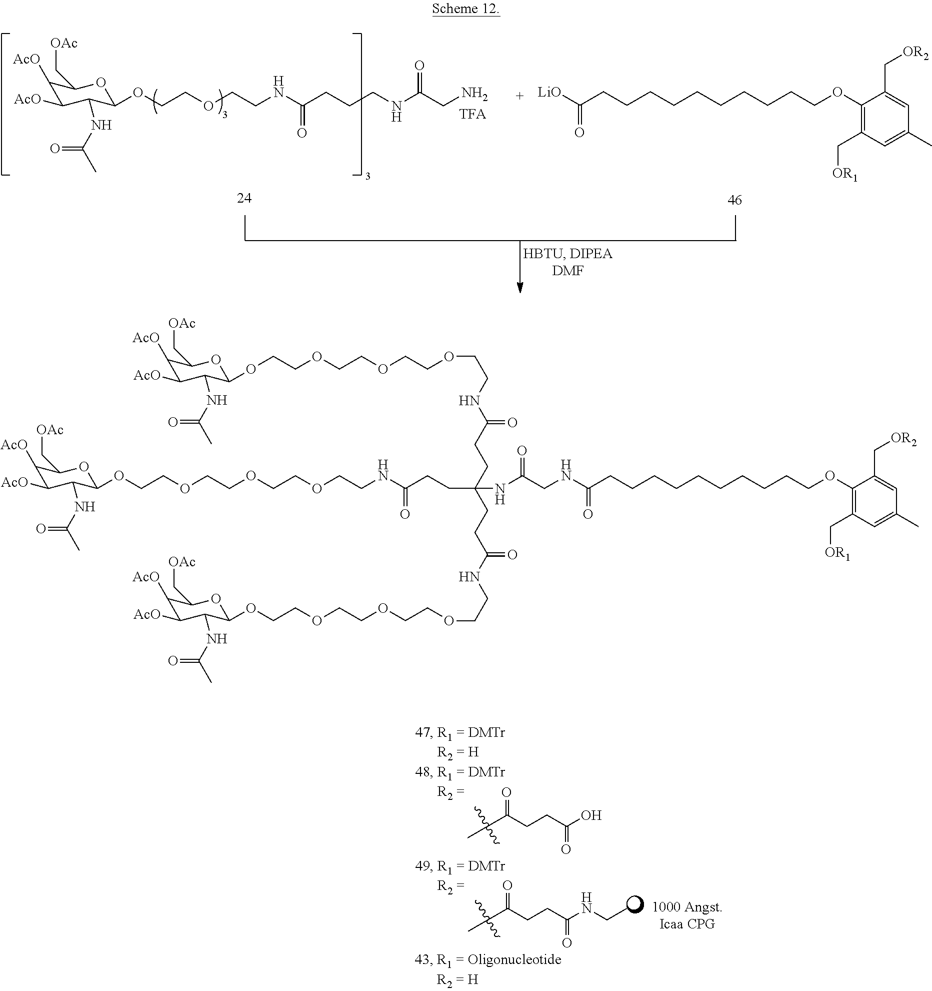

### Step 1. Preparation of methyl 11-(2,6-bis(hydroxymethyl)-4-methylphenoxy)undecanoate 44

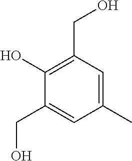

To a solution of 2,6-bis(hydroxymethyl)-p-cresol (2.7 g, 16.3 mmol), methyl 11-bromoundecanoate (5.0 g, 17.9 mmol) and potassium carbonate (4.5 g, 32.6 mmol) in acetone (100 mL) was refluxed for 16 hours. Upon completion the solution was concentrated in vacuo to dryness, suspended in ethyl acetate (150 mL) and washed with water (2×100 mL) and brine (100 mL). The ethyl acetate layer was dried on magnesium sulfate, filtered and concentrated in vacuo to dryness. The residue was purified by column chromatography on silica gel 60 (gradient 100% Hex→50% EtOAc/Hex) to afford methyl 11-(2,6-bis(hydroxymethyl)-4-methylphenoxy)undecanoate 44 as a colorless oil (1.6 g, 27%).

### Step 2. Preparation of methyl 11-(2-((bis(4-methoxyphenyl)(phenyl)methoxy)methyl)-6-(hydroxymethyl)-4-methylphenoxy)undecanoate 45

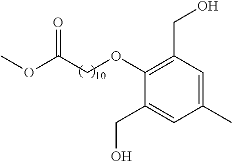

To a solution of methyl 11-(2,6-bis(hydroxymethyl)-4-methylphenoxy)undecanoate 44 (1.5 g, 4.1 mmol) in anhydrous pyridine (20 mL) was added 4,4′-Dimethoxytrityl chloride (1.4 g, 4.1 mmol). The solution was stirred overnight at room temperature. Upon completion the solution was concentrated in vacuo to dryness and purified by column chromatography on silica gel 60 (0.5 to 1% MeOH in CH₂Cl₂) to afford Methyl 11-(2-((bis(4-methoxyphenyl)(phenyl)methoxy)methyl)-6-(hydroxymethyl)-4-methylphenoxy)undecanoate 45 as a pale yellow solid (1.1 g, 40%).

### Step 3. Preparation of lithium 11-(2-((bis(4-methoxyphenyl)(phenyl)methoxy)methyl)-6-(hydroxymethyl)-4-methylphenoxy)undecanoate 46

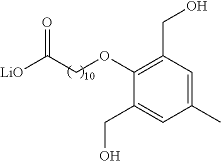

To a solution of Methyl 11-(2-((bis(4-methoxyphenyl)(phenyl)methoxy)methyl)-6-(hydroxymethyl)-4-methylphenoxy)undecanoate 45 (1.1 g, 1.7 mmol) in anhydrous tetrahydrofuran (40 mL) and water (10 mL) was added lithium hydroxide (44 mg, 1.8 mmol). The solution was concentrated in vacuo to remove all tetrahydrofuran. The remaining aqueous solution was flash frozen on liquid nitrogen then lyophilized overnight to afford lithium 11-(2-((bis(4-methoxyphenyl)(phenyl)methoxy)methyl)-6-(hydroxymethyl)-4-methylphenoxy)undecanoate 46 as a pale pink solid (1.1 g, 94%).

### Step 4. Preparation of Compound 47

A solution of 10 (1.33 g, 0.66 mmol), 46 (0.5 g, 0.73 mmol), HBTU (400 mg, 1 mmol) in N,N-dimethylformamide (25 mL) was added diisopropylethylamine (0.35 mL, 2 mmol). The solution was stirred overnight (18 hours) at room temperature. Upon completion, the solvent was remove in vacuo and the residue was purified by column chromatography on silica gel (gradient: 100% CH₂Cl₂-5%-10%-15% MeOH in CH₂Cl₂) to afford 47 as a colorless solid (710 mg, 41%).

### Step 5. Preparation of Compound 48

To a solution of 47 (0.71 g, 0.3 mmol), triethylamine (0.4 mL, 3.0 mmol) and polystyrene-DMAP (3 mmol/g loading, 200 mg, 0.6 mmol) in dichloromethane (15 mL) was added succinic anhydride (60 mg, 0.6 mmol). The solution was stirred overnight at room temperature and upon completion filtered and concentrated in vacuo to dryness. The residue was purified by column chromatography on silica gel 60 (gradient 5% to 20% MeOH in CH₂Cl₂) to afford the 48 as a pale yellow solid (570 mg, 70%). ¹H NMR (DMSO-d₆, 400 MHz) δ 7.91 (m, 1H), 7.86-7.76 (m, 6H), 7.45-7.40 (m, 2H), 7.36-7.14 (m, 10H), 7.10 (s, 1H), 6.91 (d, J=8.9 Hz, 4H), 5.21 (d, J=3.3 Hz, 3H), 5.01 (s, 2H), 4.97 (dd, J=11.2, 3.4 Hz, 3H), 4.56 (d, J=8.5 Hz, 3H), 4.06-3.98 (m, 11H), 3.93-3.84 (m, 3H), 3.81-3.72 (m, 3H), 3.74 (s, 6H), 3.65-3.46 (m, 38H), 3.40-3.35 (m, 6H), 3.20-3.16 (m, 6H), 2.56-2.44 (m, 4H), 2.33 (s, 3H), 2.15-2.08 (m, 2H), 2.10 (s, 9H), 2.04-1.96 (m, 6H), 1.89 (s, 9H), 1.82-1.76 (m, 4H), 1.77 (s, 9H), 1.54-1.34 (m, 4H), 1.28-1.10 (m, 12H),

### Step 6. Preparation of Compound 49

To a solution of 48 (100 mg, 40 μmol), N-Hydroxysuccinimide (30 mg/mL soln in acetonitrile, 50 μL, 13 μmol), N,N-Diisopropylcarbodiimide (40 μL, 264 μmol) and pyridine (50 μL) in dichloromethane (2 mL) and acetonitrile (3 mL) was added 1000 Å lcaa CPG (prime synthesis, 920 mg). The solution was stirred overnight at room temperature on an orbital shaker. TLC analysis of the reaction solution showed only partial consumption of the activated N-Hydroxysuccinic ester so additional CPG (500 mg) was added. The solution was stirred again overnight. Upon completion, the CPG was filtered and washed with dichloromethane (25 mL), acetonitrile (25 mL) and tetrahydrofuran (25 mL). The unreacted amine residues on the CPG were acetylated (capped) by adding a 1:1 solution of acetic anhydride in acetonitrile (3 mL) and 10% N-methylimidazole/10% pryridine in tetrahydrofuran (3 mL). The suspension was left for 2 hours then filtered and rinsed with equal parts tetrahydrofuran (25 mL), acetonitrile (25 mL) and dichloromethane (25 mL). The loaded CPG 49 was dried under high vacuum overnight. The ligand loading efficiency was determined to be 22 μmole/g using a standard DMT loading assay (3% trichloroacetic acid in CH₂Cl₂, UV-VIS, A₅₀₄).

### Step 7. Preparation of Conjugate 43

The resulting GalNAc loaded CPG solid support 49 was employed in automated oligonucleotide synthesis using standard procedures. Nucleotide deprotection followed by removal from the solid support (with concurrent galactosamine acetate deprotection) afforded a GalNAc-oligonucleotide conjugate 43.

### Example 6. Synthesis of Conjugate 50

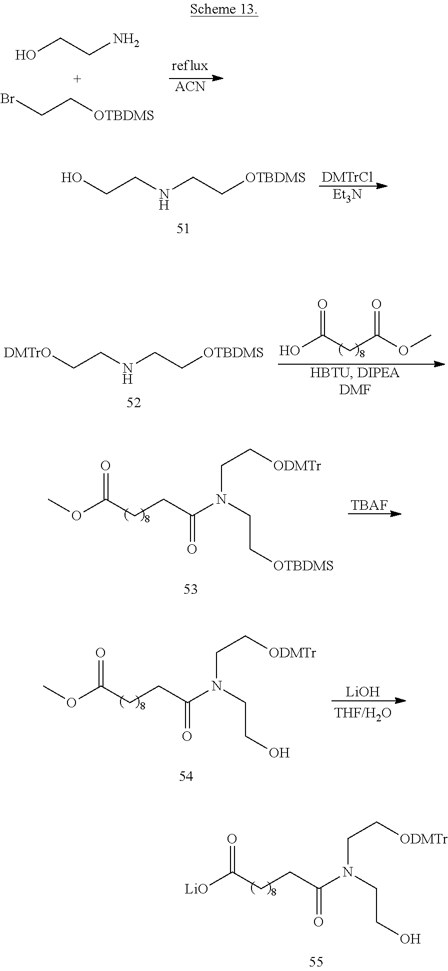

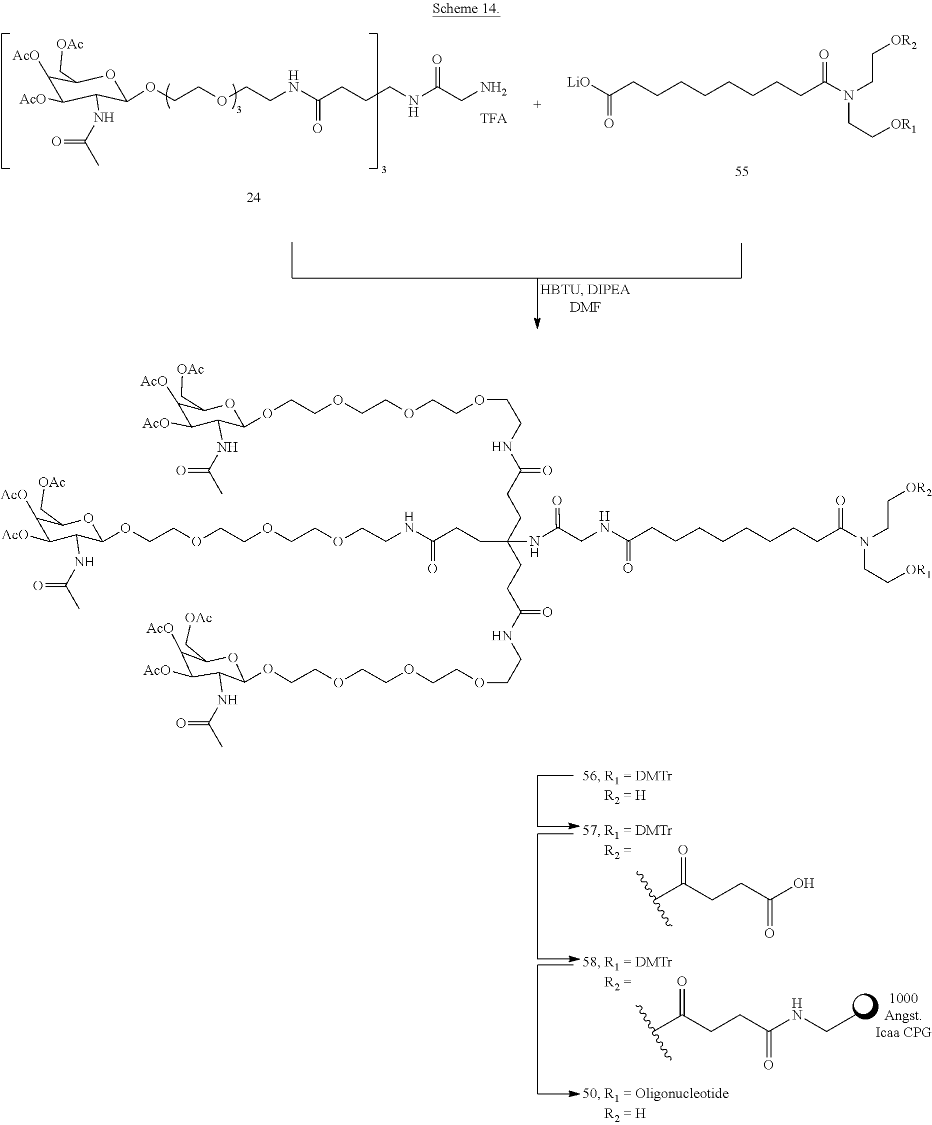

### Step 1. Preparation of 2-((2-((tert-butyldimethylsilyl)oxy)ethyl)amino)ethan-1-ol 51

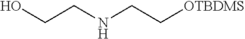 [SMILES: CC(C)(C)[Si](C)(C)OCCNCCO]

A solution of ethanolamine (77 mL, 1.25 mol) and (2-bromoethoxy)-tert-butyl dimethylsilane (15 g, 62.7 mmol) in anhydrous acetonitrile (200 mL) was refluxed for 3 hours. Upon completion the reaction was cooled to room temperature, diluted with water (400 mL) and extracted with ethyl acetate (3×150 mL). The combined ethyl acetate extracts were dried on magnesium sulfate, filtered and concentrated in vacuo to dryness. The residue was purified by filtration through a pad of silica first with 50% ethyl acetate/hexanes then 50% MeOH/EtOAc to afford 51 as a pale yellow oil (14 g, 100%).

### Step 2. Preparation of 2-(bis(4-methoxyphenyl)(phenyl)methoxy)-N-(2-((tert-butyldimethylsilyl)oxy)ethyl)ethan-1-amine 52

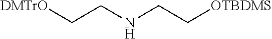

To a solution of 2-((2-((tert-butyldimethylsilyl)oxy)ethyl)amino)ethan-1-ol 51 (14 g, 64 mmol) and triethylamine (17.5 mL, 128 mmol) in anhydrous dichloromethane (250 mL) was added 4,4′-Dimethoxytrityl chloride (24 g, 70 mmol). The solution was stirred overnight at room temperature then concentrated in vacuo to dryness. The residue was dissolved in ethyl acetate (300 mL) and washed with water (250 mL) and brine (250 mL). The ethyl acetate was dried on magnesium sulfate, filtered and concentrated in vacuo to dryness. Purification by column chromatography on silica gel 60 (1% to 5% MeOH in CH₂Cl₂) afforded 52 as a pale yellow viscous oil (13 g, 39%).

### Step 3. Preparation of methyl 10-((2-(bis(4-methoxyphenyl)(phenyl)methoxy)ethyl)(2-((tert-butyldimethylsilyl)oxy)ethyl)amino)-10-oxodecanoate 53

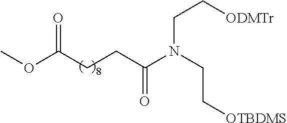

A solution of 2-(bis(4-methoxyphenyl)(phenyl)methoxy)-N-(2-((tert-butyldimethylsilyl)oxy)ethyl)ethan-1-amine 52 (5.4 g, 10.3 mmol), monomethyl sebacate (2.2 g, 10.3 g), HBTU (4.9 g, 12.9 mmol), DIPEA (5.3 mL, 30.9 mmol) in N,N-dimethylformamide (100 mL) was stirred for 3 hours at room temperature. Upon completion, the solution was poured into water (400 mL) and extracted with ethyl acetate (1×500 mL). The ethyl acetate extract was washed with brine (2×250 mL), dried on magnesium sulfate, filtered and concentrated in vacuo to dryness. Purification by column chromatography on silica gel 60 (10% to 25% ethyl acetate in hexanes) afforded 53 as a viscous yellow oil (6.5 g, 87%).

### Step 4. Preparation of methyl 10-((2-(bis(4-methoxyphenyl)(phenyl)methoxy)ethyl)(2-hydroxyethyl)amino)-10-oxodecanoate 54

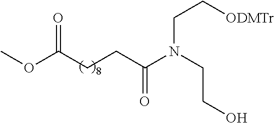

To a solution of methyl 10-((2-(bis(4-methoxyphenyl)(phenyl)methoxy)ethyl)(2-((tert-butyldimethylsilyl)oxy)ethyl)amino)-10-oxodecanoate 53 (2.0 g, 2.8 mmol) and triethylamine (1 mL) in anhydrous tetrahydrofuran (20 mL) was added TBAF (1M in THF, 3.4 mL, 3.3 mmol). The solution was stirred for 6h, but only partial conversion observed by TLC (5% MeOH in CH₂Cl₂). Additional 1.7 mL TBAF added and the solution was stirred overnight at room temperature. Upon completion, the solution was concentrated in vacuo and purified by column chromatography on silica gel 60 (10% to 50% EtOAc in hexanes then 100% EtOAc) to afford 54 as a viscous colorless oil (0.5 g, 29%).

### Step 5. Preparation of lithium 10-((2-(bis(4-methoxyphenyl)(phenyl)methoxy)ethyl)(2-hydroxyethyl)amino)-10-oxodecanoate 55

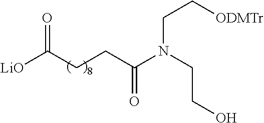

To a solution of methyl 10-((2-(bis(4-methoxyphenyl)(phenyl)methoxy)ethyl)(2-hydroxyethyl)amino)-10-oxodecanoate 54 (0.5 g, 0.83 mmol) in THF (40 mL) was added water (10 mL) and lithium hydroxide (24 mg, 1.0 mmol). The solution was stirred overnight at room temperature then concentrated in vacuo to remove the THF. The remaining aqueous solution was flash frozen on liquid nitrogen and lyophilized to afford 55 as a colorless solid (485 mg, 95%).

### Step 6. Preparation of Compounds 56, 57, 58 and 50

Compounds 56, 57, 58 and 50 were prepared using the identical procedures to those used to synthesize compounds 47, 48, 49 and 43 respectfully.

### Example 7. Synthesis of Conjugate 59

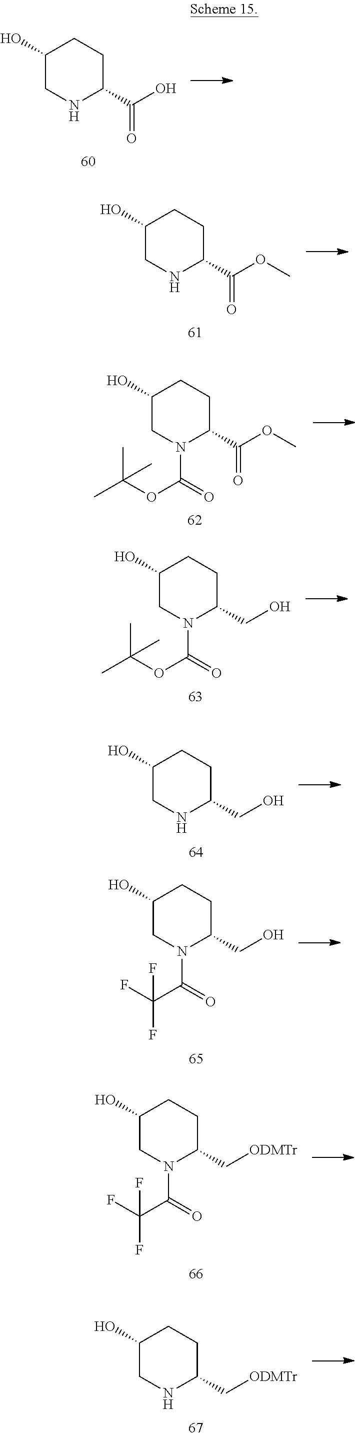

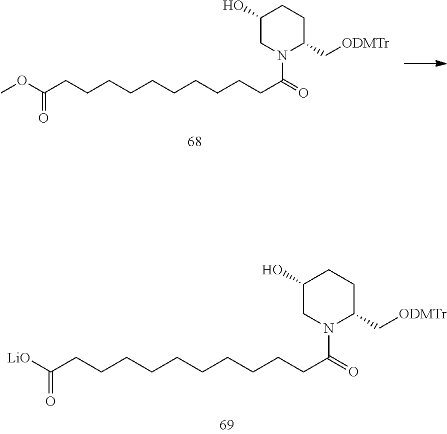

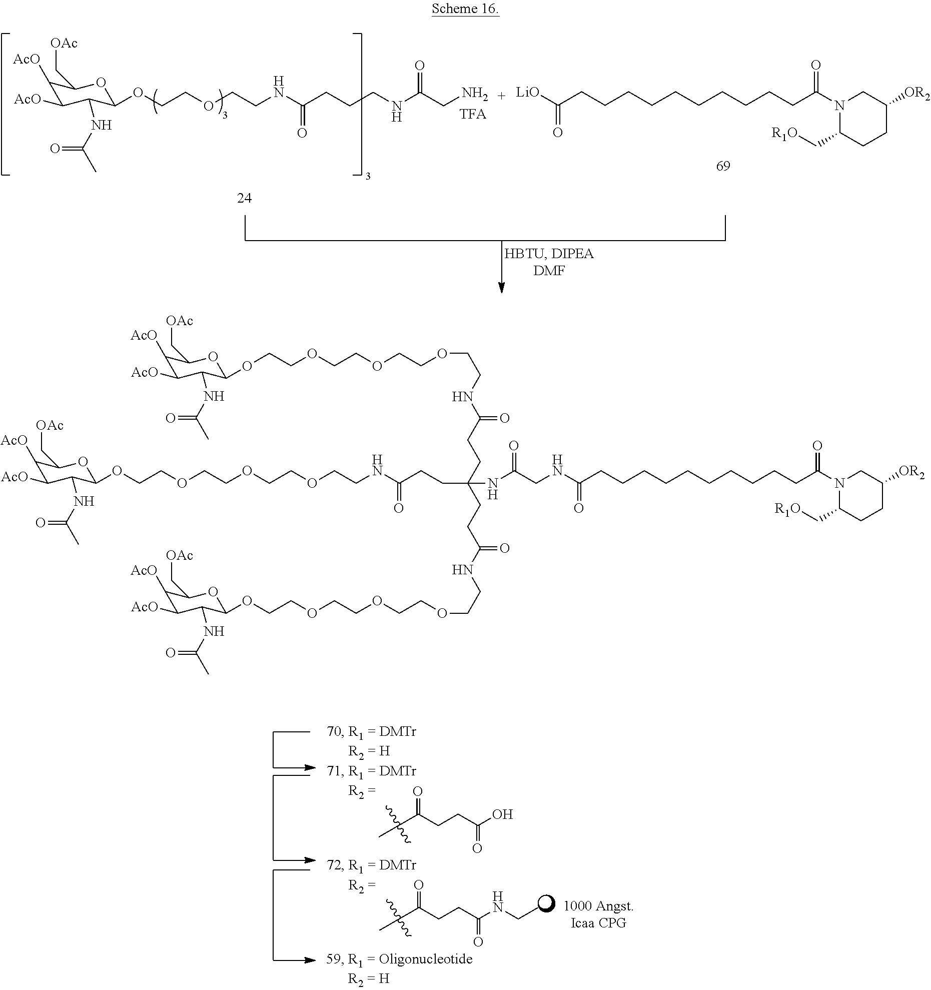

### Step 1. Preparation of methyl (2R,5R)-5-hydroxypiperidine-2-carboxylate 61

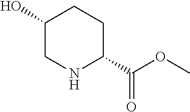

(2R,5R)-5-hydroxypiperidine-2-carboxylic acid 60 (3.5 g, 24.1 mmol) was stirred in MeOH (50 mL). HCl (g) was bubbled through the solution for 2 mins and the reaction stirred at reflux for 1.5 h. The reaction was concentrated in-vacuo to give methyl (2R,5R)-5-hydroxypiperidine-2-carboxylate 61 in quantitative yield which was used without further purification.

### Step 2. Preparation of 1-(tert-butyl) 2-methyl (2R,5R)-5-hydroxypiperidine-1,2-dicarboxylate 62

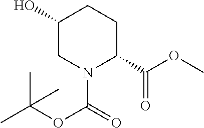 [SMILES: COC(=O)[C@H]1CC[C@@H](O)CN1C(=O)OC(C)(C)C]

Methyl (2R,5R)-5-hydroxypiperidine-2-carboxylate 61 (24.1 mmol) and TEA (7.2 mL, 53.02 mmol) were stirred in DCM (100 mL) at RT. Di-tert-butyl-di-carbonate (5.7 g, 26.5 mmol) was added in portions and the reaction stirred for 2 h. The reaction was diluted with DCM (100 mL) and washed sequentially with 1 M HCl (2×75 mL), saturated NaHCO₃ (2×75 mL), H₂O (2×75 mL) and saturated NaCl solution (2×75 mL). The organics were separated, dried (Na₂SO₄) and concentrated in-vacuo to give 1-(tert-butyl) 2-methyl (2R,5R)-5-hydroxypiperidine-1,2-dicarboxylate 62 (5.53 g, 88%) which was used without further purification.

### Step 3. Preparation of tert-butyl (2R,5R)-5-hydroxy-2-(hydroxymethyl)piperidine-1-carboxylate 63

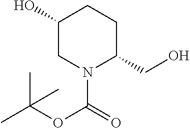 [SMILES: CC(C)(C)OC(=O)N1C[C@H](O)CC[C@@H]1CO]

(2R,5R)-1-(tert-Butoxycarbonyl)-5-hyroxypiperidine-2-carboxylic acid 62 (5.53 g, 21.4 mmol) was stirred in THF at 0° C. LiBH₄ (3.0 M solution in THF) (8.9 mL, 27.7 mmol) was added dropwise over 1 hr. The reaction was allowed to warm to RT and stirring continued for 16 h. Reaction was quenched with 1M NaOH, THF removed in-vacuo and the aqueous exhaustively extracted with EtOAc (10×100 mL). The combined organics were washed with H₂O (50 mL), saturated NaCl solution (2×50 mL), dried (Na₂SO₄) and concentrated in-vacuo to give tert-butyl (2R,5R)-5-hydroxy-2-(hydroxymethyl)piperidine-1-carboxylate 63 (2.4 g, 49.0%) which was used without further purification.

### Step 4. Preparation of (3R,6R)-6-(hydroxymethyl)piperidin-3-ol 64

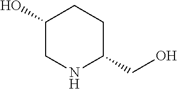

tert-Butyl (2R,5R)-5-hydroxy-2-(hydroxymethyl)piperidine-1-carboxylate 63 (2.4 g, 10.4 mmol) was stirred in Et₂O at RT. HCl (g) was bubbled through for 45 secs and the reaction stirred at RT for 45 mins. The reaction was concentrated in-vacuo and dried under hi-vac to afford (3R,6R)-6-(hydroxymethyl)piperidin-3-ol 64. The product was used without further purification.

### Step 5. Preparation of 2,2,2-trifluoro-1-((2R,5R)-5-hydroxy-2-(hydroxymethyl)piperidin-1-yl)ethan-1-one 65

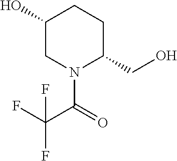 [SMILES: O=C(N1C[C@H](O)CC[C@@H]1CO)C(F)(F)F]

Crude (3R,6R)-6-(hydroxymethyl)piperidin-3-ol 64 from the previous reaction was stirred in MeCN (50 mL) with TEA (3.5 mL, 25.2 mmol) at RT. Ethyl trifluoroacetate (3 mL, 25.2 mmol) was added and the reaction stirred at RT for 16 hr, then concentrated in-vacuo to give 2,2,2-trifluoro-1-((2R,5R)-5-hydroxy-2-(hydroxymethyl)piperidin-1-yl)ethan-1-one 65. The product was used without further purification.

### Step 6. Preparation of 1-((2R,5R)-2-((bis(4-methoxyphenyl)(phenyl)methoxy)methyl)-5-hydroxypiperidin-1-yl)-2,2,2-trifluoroethan-1-one 66

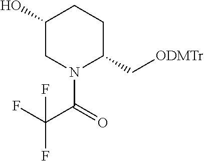

Crude 2,2,2-trifluoro-1-((2R,5R)-5-hydroxy-2-(hydroxymethyl)piperidin-1-yl)ethan-1-one 65 from the previous reaction was stirred in DCM with TEA (50 mL) at RT. 4,4′-Dimethoxytrityl chloride (DMTrCl) (3.87 g, 11.44 mmol) was added in one portion and the reaction stirred at RT for 3 hours. The reaction was diluted with DCM (50 mL) and washed sequentially with saturated NaHCO₃ (2×75 mL), H₂O (2×75 mL) and saturated NaCl solution (2×75 mL). The organics were separated, dried (Na₂SO₄), concentrated in-vacuo and purified by column chromatography (100% hexanes-60% EtOAc/Hexanes) (0.1% TEA) to give 1-((2R,5R)-2-((bis(4-methoxyphenyl)(phenyl)methoxy)methyl)-5-hydroxypiperidin-1-yl)-2,2,2-trifluoroethan-1-one 66 (3.14 g, 57%)

### Step 7. Preparation of (3R,6R)-6-((bis(4-methoxyphenyl)(phenyl)methoxy)methyl)-piperidin-3-ol 67

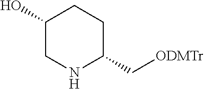

1-((2R,5R)-2-((bis(4-methoxyphenyl)(phenyl)methoxy)methyl)-5-hydroxypiperidin-1-yl)-2,2,2-trifluoroethan-1-one 66 (3.14 g, 6.0 mmol) was stirred in MeOH (50 mL) at RT. KOH (672 mg, 12 mmol) was added and the reaction stirred at RT for 16 hours. Additional KOH (300 mg, 6 mmol) was added and stirring continued for an additional 24 h. The reaction was concentrated in-vacuo, taken up in DCM (150 mL), washed with H₂O (4×50 mL), dried (Na₂SO₄) and concentrated in-vacuo to give (3R,6R)-6-((bis(4-methoxyphenyl)(phenyl)methoxy)methyl)piperidin-3-ol 67 (2.34 g, 90%) which was used without further purification.

### Step 8. Preparation of methyl 12-((2R,5R)-2-((bis(4-methoxyphenyl)(phenyl)-methoxy)methyl)-5-hydroxypiperidin-1-yl)-12-oxododecanoate 68

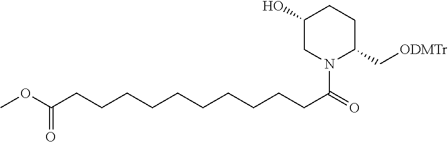

(3R,6R)-6-((Bis(4-methoxyphenyl)(phenyl)methoxy)methyl)piperidin-3-ol 67 (2.34 g, 5.34 mmol) was stirred in DCM (75 mL) at RT. Triethylamine (2.2 mL, 16.2 mmol), HATU (3.5 g, 9.2 mmol) and 12-methoxy-12-oxododecanoic acid (1.32 g, 5.4 mmol) were added and the reaction stirred at RT for 3 h. The resultant solid precipitate was removed by filtration, the filtrate concentrated in-vacuo and the residue purified by column chromatography (2.5% MeOH/DCM, 0.1% TEA) to give methyl 12-((2R,5R)-2-((bis(4-methoxyphenyl)(phenyl)methoxy)methyl)-5-hydroxypiperidin-1-yl)-12-oxododecanoate 68 in quantitative yield.

### Step 9. Preparation of lithium 12-((2R,5R)-2-((bis(4-methoxyphenyl)(phenyl)methoxy)-methyl)-5-hydroxypiperidin-1-yl)-12-oxododecanoate 69

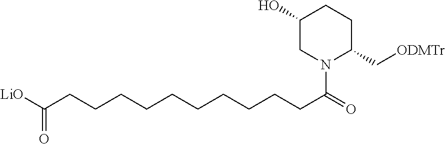

Methyl 12-((2R,5R)-2-((bis(4-methoxyphenyl)(phenyl)methoxy)-5-hydroxypiperidin-1-yl)-12-oxododecanoate 68 (5.4 mmol) and LiOH (140 mg, 5.94 mmol) were stirred in THF:H₂O (1:1, 100 mL) at RT for 48 h. The THF was removed in-vacuo, the aqueous frozen and lyophilized to give lithium 12-((2R,5R)-2-((bis(4-methoxyphenyl)(phenyl)methoxy)methyl)-5-hydroxypiperidin-1-yl)-12-oxododecanoate 69 (3.2 g, 91%). Which was used in subsequent reactions without additional purification.

### Step 10. Preparation of Compounds 70, 71, 72, and 59

Compounds 70, 71, 72 and 59 were prepared using the identical procedures to those used to synthesize compounds 47, 48, 49 and 43 respectfully.

### Example 8. Synthesis of Conjugate 142

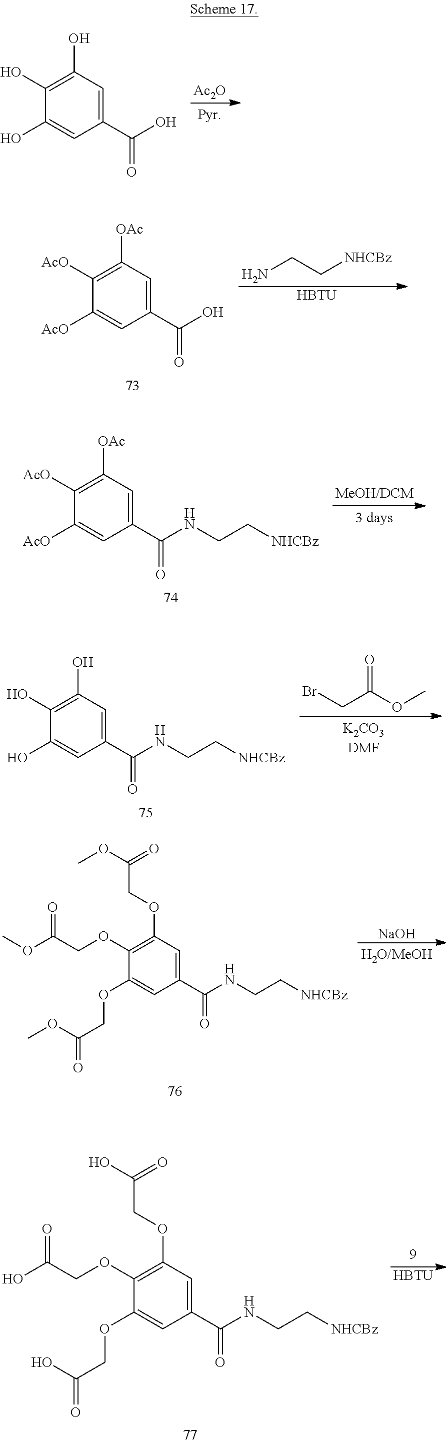

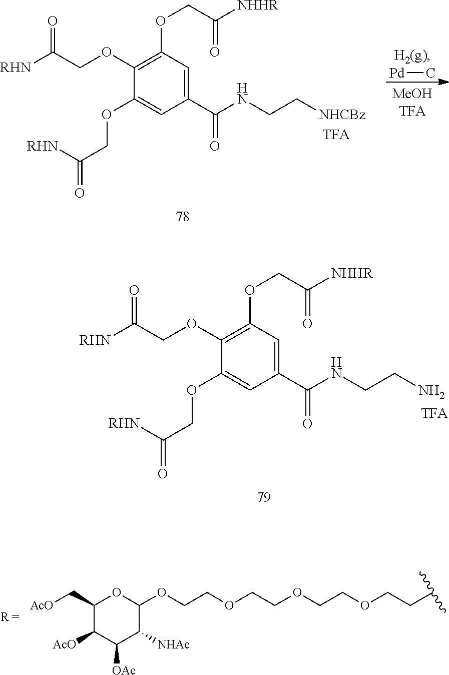

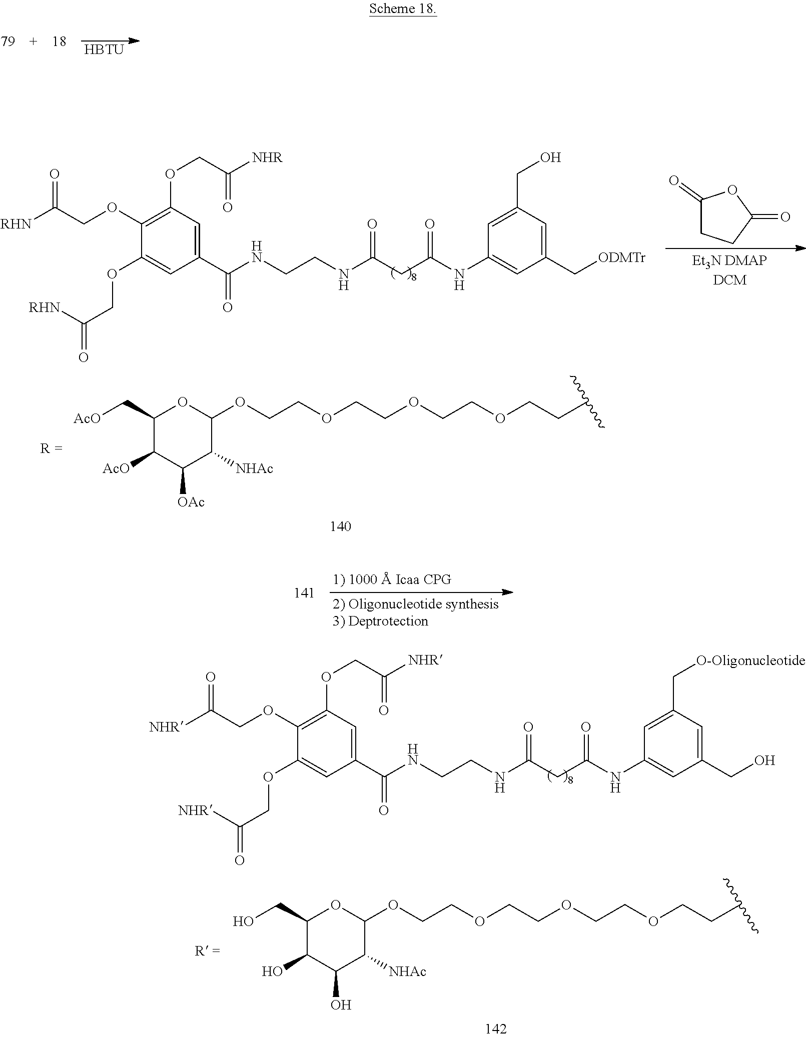

### Step 1. Preparation of 3,4,5-Triacetoxybenzoic acid 73

To a solution of Gallic acid (20 g) in pyridine (50 mL) and acetic anhydride (50 mL). The solution was stirred overnight at room temperature then poured into ice water (1 L). The solution was made acidic with concentrated hydrochloric acid where upon a colorless solid precipitated. The solid was collected via filtration and washed with water (5×100 mL). The wet solid was frozen on liquid nitrogen and freeze dried to afford 3,4,5-triacetoxybenzoic acid (26 g, 75%).

### Step 2. Preparation of 5-((2-((2-Oxo-2-phenyl-1λ²-ethyl)amino)ethyl)carbamoyl)benzene-1,2,3-triyl triacetate 74

To a solution of 3,4,5-triacetoxybenzoic acid (10 g, 33.8 mmol), N-carbobenzoxy-1,2-diaminoethane hydrochloride (5.3 g, 33.8 mmol) and HBTU (13.5 g, 35.5 mmol) in DMF (200 mL) was added DIPEA (17.5 mL, 101 mmol). The solution was stirred for 16 hours then diluted with ethyl acetate (250 mL), washed with brine (3×200 mL), dried on magnesium sulfate, filtered and concentrated in vacuo to dryness. The crude product was purified by column chromatography on silica gel (Gradient 1% to 5% MeOH in DCM) to afford 5-((2-((2-Oxo-2-phenyl-1λ²-ethyl)amino)ethyl)carbamoyl)benzene-1,2,3-triyl triacetate as an off white solid (5.5 g).

### Step 3. Preparation of 3,4,5-Trihydroxy-N-(2-((2-oxo-2-phenyl-1λ²-ethyl)amino)ethyl)benzamide 75

A solution of 5-((2-((2-Oxo-2-phenyl-1λ²-ethyl)amino)ethyl)carbamoyl)benzene-1,2,3-triyl triacetate (5 g, 1.1 mmol) in 1:1 MeOH/CH₂Cl₂ (100 mL) was stirred for 3 days at room temperature. Upon completion the solvent was removed to afford 3,4,5-Trihydroxy-N-(2-((2-oxo-2-phenyl-1λ²-ethyl)amino)ethyl)benzamide as a colorless solid (4 g, quantitative).

### Step 4. Preparation of Trimethyl 2,2′,2″-((5-((2-((2-oxo-2-phenyl-1λ²-ethyl)amino)ethyl)carbamoyl)benzene-1,2,3-triyl)tris(oxy))triacetate 76

A solution of 3,4,5-Trihydroxy-N-(2-((2-oxo-2-phenyl-1λ²-ethyl)amino)ethyl)benzamide (4 g, 11.6 mmol), methyl bromoacetate (7.7 g, 46.4 mmol) and potassium carbonate (9.6 g, 69.4 mmol) in DMF (100 mL) was stirred overnight at 60° C. Upon completion the solution was cooled to room temperature, diluted with ethyl acetate (200 mL), washed with water (200 mL), brine (3×100 mL), dried on magnesium sulfate, filtered and concentrated in vacuo to dryness. The crude product was purified by column chromatography on silica gel (Gradient 2% to 10% MeOH in DCM) to afford trimethyl 2,2′,2″-((5-((2-((2-oxo-2-phenyl-1λ²-ethyl)amino)ethyl)carbamoyl)benzene-1,2,3-triyl)tris(oxy))-triacetate as a beige solid (5 g, 79%)

### Step 5. Preparation of 2,2′,2″-((5-((2-((2-Oxo-2-phenyl-1λ²-ethyl)amino)ethyl)-carbamoyl)benzene-1,2,3-triyl)tris(oxy))triacetic acid 77

A solution of trimethyl 2,2′,2″-((5-((2-((2-oxo-2-phenyl-1λ²-ethyl)amino)ethyl)-carbamoyl)benzene-1,2,3-triyl)tris(oxy))triacetate (5 g, 9.2 mmol) and 1M NaOH (30 mL) in methanol (100 mL) was stirred for 2 hours at room temperature. Upon completion the reaction was concentrated to remove the methanol and diluted with water (75 mL). The mixture was cooled to 0° C., acidified with 2M HCl and extracted with ethyl acetate (5×150 mL). The combined ethyl acetate extracts were dried on magnesium sulfate, filtered and concentrated in vacuo to dryness to afford 2,2′,2″-((5-((2-((2-Oxo-2-phenyl-1λ²-ethyl)amino)ethyl)carbamoyl)benzene-1,2,3-triyl)tris(oxy))triacetic acid as a colorless solid (2.3 g, 50%).

### Step 6. Preparation of Compound 78

Compound 78 was prepared from compounds 9 (2.75 g, 4.3 mmol) and 77 (0.5 g, 0.96 mmol) using an identical procedure to that used for compound 13. Yield: 600 mg.

### Step 7. Preparation of Compound 79

Compound 79 was prepared from compounds 78 (0.6 g) using an identical procedure to that used for compound 14. Yield: 500 mg.

### Step 8. Preparation of Compound 140

Compound 140 was prepared from compound 79 (500 mg, 0.25 mmol) and compound 18 (175 mg, 0.25 mmol) using an identical procedure to that used for compound 19. Yield: 250 mg, 44%.

### Step 9. Preparation of Compound 141

Compound 141 was prepared from compound 140 (250 mg, 0.11 mmol) using an identical procedure to that used for compound 20. Yield: 200 mg.

### Step 10. Preparation of Conjugate 142

Conjugate 142 was prepared from compound 141 (200 mg) and 1000 A lcaa CPG (1.8 g) using an identical procedure to that used for compound 1. Yield: 1.9 g, 22 μmol/g CPG loading. The resulting GalNAc loaded CPG solid support was employed in automated oligonucleotide synthesis using standard procedures. Nucleotide deprotection followed by removal from the solid support (with concurrent galactosamine acetate deprotection) afforded the GalNAc-oligonucleotide conjugate 142.

### Example 9. Synthesis of Conjugate 145

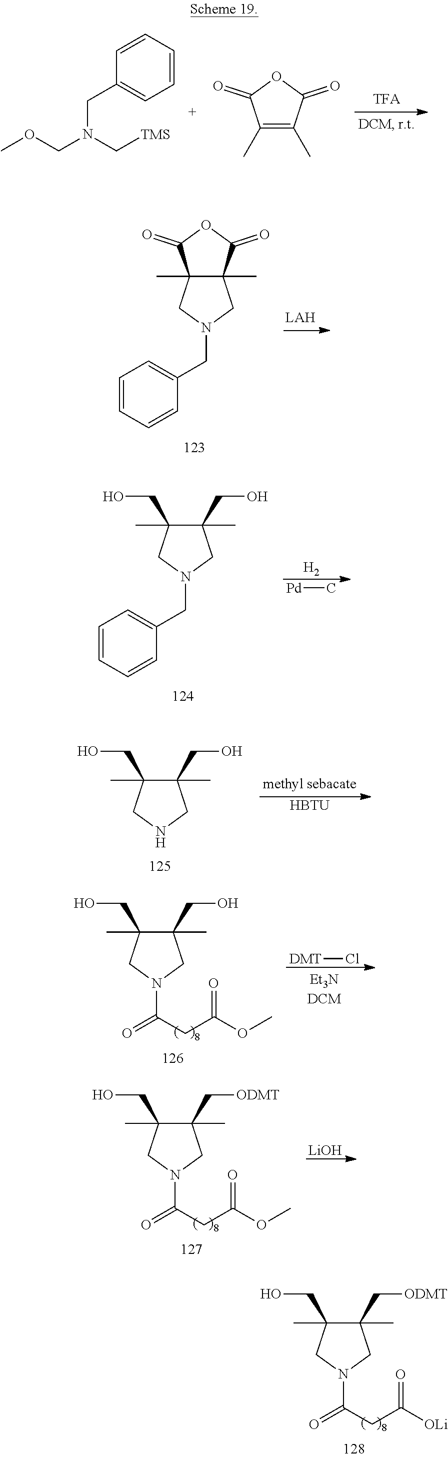

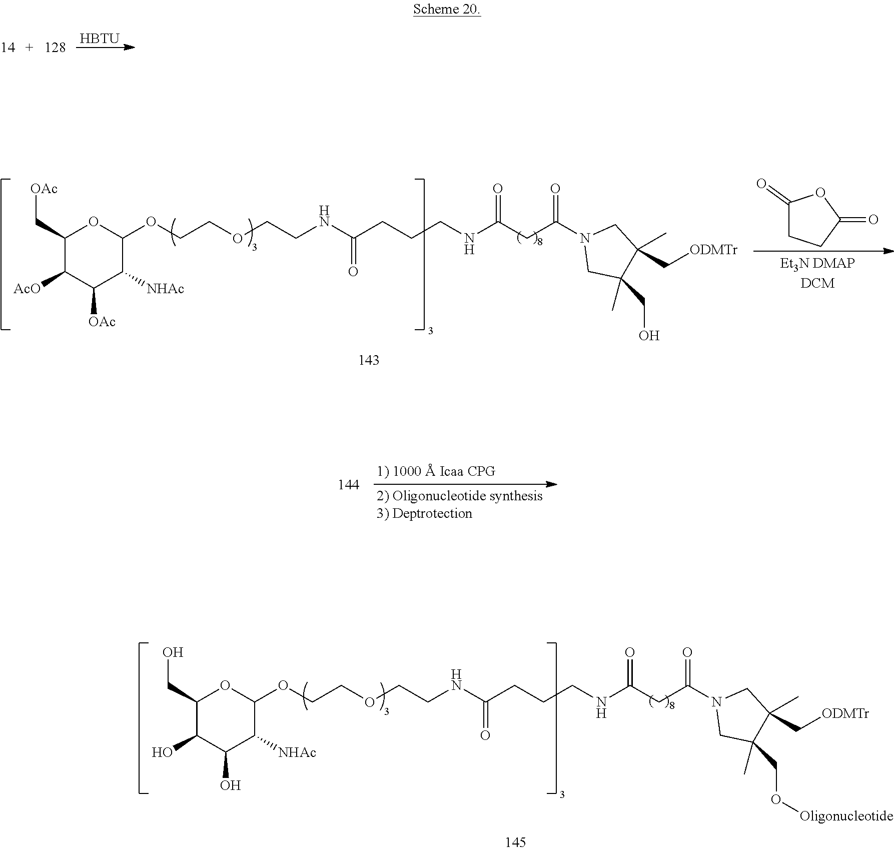

### Step 1. Preparation of Racemic (cis) 5-Benzyl-3a,6a-dimethyltetrahydro-1H-furo[3,4-c]pyrrole-1,3(3aH)-dione 123

To a cooled solution (0° C.) of 3,4-dimethylfuran-2,5-dione (3 g, 24 mmol) and N-benzyl-1-methoxy-N-((trimethylsilyl)methyl)methanamine (7 g, 29.8 mmol) in dichloromethane (75 mL) was slowly added trifluoroacetic acid (75 μL). Stir overnight allowing the solution to slowly warm to room temperature as the ice bath melted. The reaction mixture was concentrated to dryness, dissolved in ethyl acetate (100 mL), washed with saturated sodium bicarbonate (2×100 mL), dried on magnesium sulfate, filtered and concentrated to dryness. Purification by column chromatography on silica gel (gradient: 20% ethyl acetate in hexanes to 100% ethyl acetate) afforded racemic (cis) 5-Benzyl-3a,6a-dimethyltetrahydro-1H-furo[3,4-c]pyrrole-1,3(3aH)-dione as a yellow oil (3.5 g, 56%).

### Step 2. Preparation of Racemic (cis) 1-Benzyl-3,4-dimethylpyrrolidine-3,4-diyl)dimethanol 124

To a cooled (0° C.) solution of (3aR,6aS)-5-Benzyl-3a,6a-dimethyltetrahydro-1H-furo[3,4-c]pyrrole-1,3(3aH)-dione (3.5 g, 13.4 mmol) in anhydrous diethyl ether (50 mL) was added slowly lithium aluminum hydride pellets (1.5 g, 40 mmol) over three portions. The solution was stirred overnight warming to room temperature as the ice water bath melted. Upon completion, the reaction was cooled to 0° C. and very slowly quenched with 1.5 mL of 5M NaOH followed by 1.5 mL of water. Stir for 30 minutes then add magnesium sulfate and filter. The filtrate was concentrated to afford racemic (cis) 1-Benzyl-3,4-dimethylpyrrolidine-3,4-diyl)dimethanol as a colorless oil (2.7 g).

### Step 3. Preparation of Racemic (cis) 3,4-Dimethylpyrrolidine-3,4-diyl)dimethanol 125

To a solution of ((3R,4S)-1-Benzyl-3,4-dimethylpyrrolidine-3,4-diyl)dimethanol (10 g, 40 mmol) in methanol (10 mL) was added 10% palladium on activated charcoal wet (1 g). The solution was stirred vigorously under a hydrogen atmosphere for 16 hours. Upon completion the solution was filtered through Celite, and concentrated to dryness to afford racemic (cis) 3,4-Dimethylpyrrolidine-3,4-diyl)dimethanol as a colorless solid (5.5 g, 86%).

### Step 4. Preparation of Racemic (cis) Methyl 10-(3,4-bis(hydroxymethyl)-3,4-dimethylpyrrolidin-1-yl)-10-oxodecanoate 126

Compound 126 was prepared from compound 125 (1.3 g, 8.2 mmol) and monomethyl sebacate (1.8 g, 8.2 mmol) using an identical procedure to that used for compound 17. Yield: 1.8 g, 61%.

### Step 5. Preparation of Racemic (cis) Methyl 10-(3-((bis(4-methoxyphenyl-)(phenyl)methoxy)-methyl)-4-(hydroxymethyl)-3,4-dimethylpyrrolidin-1-yl)-10-oxodecanoate 127

Compound 127 was prepared from compound 126 (1.8 g, 5.0 mmol) and 4,4′-Dimethoxytrityl chloride (1.7 g, 5.0 mmol) using an identical procedure to that used for compound 18. Yield: 1.4 g, 42%.

### Step 6. Preparation of Racemic (cis) Lithium 10-(3-((bis(4-methoxyphenyl)-(phenyl)methoxy)-methyl)-4-(hydroxymethyl)-3,4-dimethylpyrrolidin-1-yl)-10-oxodecanoate 128

To a solution of compound 127 (3.0 g, 4.6 mmol) in THF (50 mL) and water (50 mL) was added lithium hydroxide (121 mg, 5.0 mmol). The solution was stirred for 4 hours at room temperature then concentrated to remove the THF. The remaining aqueous solution was freeze dried overnight to afford a pale pink solid (2.9 g, quantitative).

### Step 7. Preparation of Compound 143

Compound 143 was prepared from compound 128 (270 mg, 0.42 mmol) and compound 14 (800 mg, 0.42 mmol) using an identical procedure to that used for compound 19. Yield: 900 mg, 87%.

### Step 8. Preparation of Compound 144

Compound 144 was prepared from compound 143 (500 mg, 0.2 mmol) using an identical procedure to that used for compound 20. Yield: 200 mg.

### Step 9. Preparation of Conjugate 145

Conjugate 145 was prepared from compound 144 (200 mg) and 1000 A lcaa CPG (1.8 g) using an identical procedure to that used for compound 1. Yield: 1.9 g, 20 μmol/g CPG loading. The resulting GalNAc loaded CPG solid support was employed in automated oligonucleotide synthesis using standard procedures. Nucleotide deprotection followed by removal from the solid support (with concurrent galactosamine acetate deprotection) afforded the GalNAc-oligonucleotide conjugate 145.

### Example 10. Synthesis of Conjugate 150

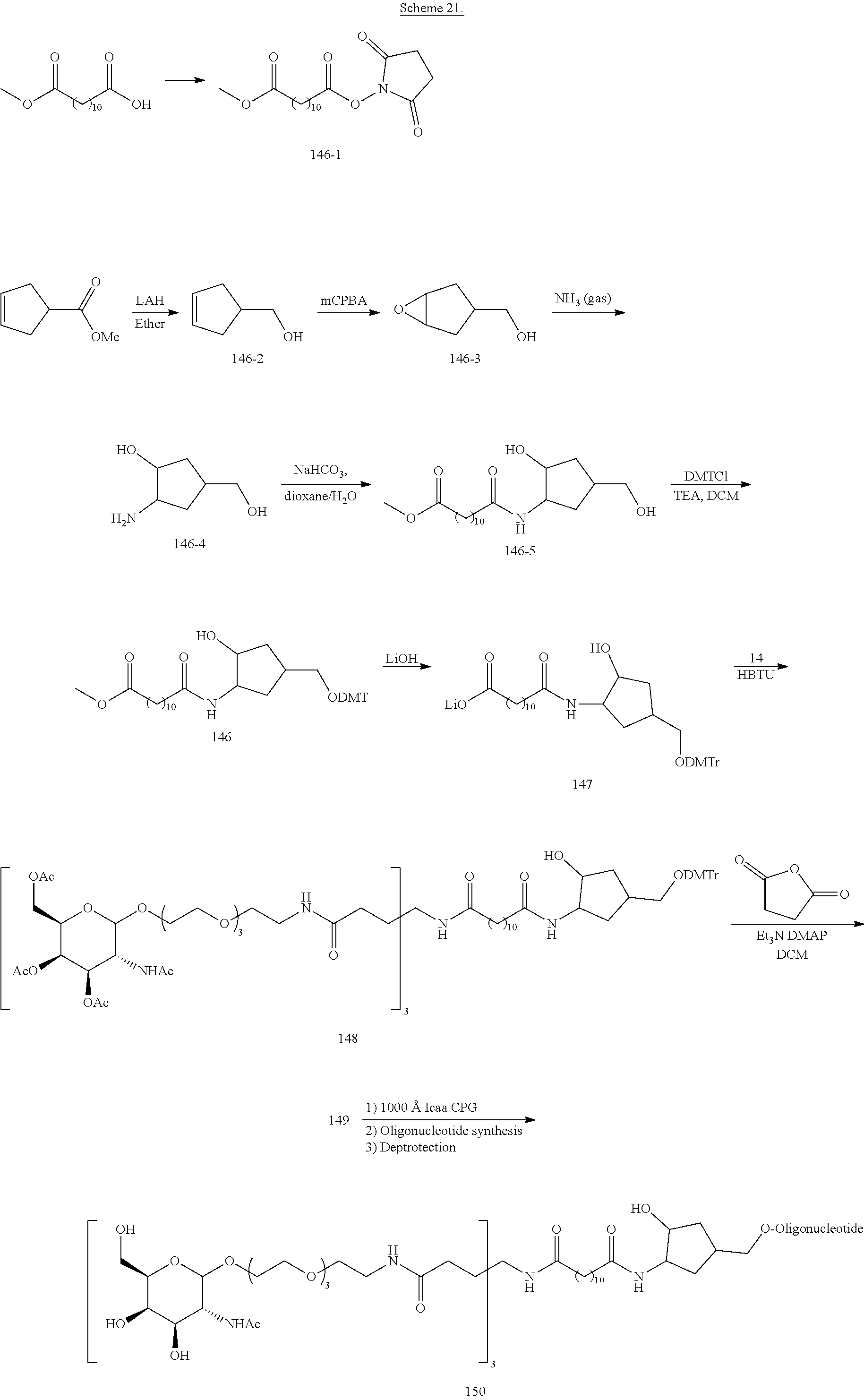

### Step 1. Preparation of 146-1

To a solution of mono methyl ester of dodecanedioic acid (12.2 g, 50.0 mmol) in dichloromethane (300 mL) was added N-hydroxysuccinimide (6.10 g, 53.0 mmol) and 1-ethyl-3-(3-dimethylaminopropyl)carbodiimide hydrochloride (EDC) (10.52 g, 55.0 mmol). The cloudy mixture was stirred overnight at room temperature and the reaction became a clear solution. TLC indicated the reaction was completed. The organics were washed with saturated NH₄Cl (300 mL) and brine (100 mL). The organic layer was separated, dried over MgSO4 and concentrated to dryness to pure 1-(2,5-dioxopyrrolidin-1-yl) 12-methyl dodecanedioate 146-1 as a white solid (16.7 g, 97.8%).

### Step 2. Preparation of cyclopent-3-en-1-ylmethanol 146-2

To a suspension of lithium aluminum hydride (15.2 g, 0.40 mol) in anhydrous ether (1 L) at 0° C. under nitrogen, was added the solution of methyl cyclopent-3-enecarboxylate (50 g, 0.40 mol) in ether (300 mL) dropwise over 5 hrs. The suspension was stirred at room temperature overnight. TLC indicated the completion of the reaction. The reaction was re-cooled to 0° C. Saturated solution of Na₂SO₄ (32 mL) was added dropwise to quench the reaction. After the addition was complete, the mixture was stirred for another 3 hrs and was filtered through a pad of celite. Evaporation of solvent afforded cyclopent-3-enylmethanol 146-2 (37.3 g, 95%) as a colorless liquid.

### Step 3. Preparation of (6-oxabicyclo[3.1.0]hexan-3-yl)methanol 146-3

To a solution of cyclopent-3-enylmethanol 146-2 (4.0 g, 41 mmol) in dichloromethane (150 mL) at 0° C. was added 3-chloroperbenzoic acid (10 g, 45 mmol, 77% purity) by portion. The reaction was stirred overnight. Dichloromethane (150 mL) was added. The organics was washed with sodium thiosulfate (12 g in 10 mL water), followed by saturated NaHCO₃ (40 mL). This was repeated till all the remaining 3-chloroperbenzoic acid was washed away. The organic was dried over MgSO4. Evaporation of solvent gave a mixture of cis- and trans-6-oxabicyclo[3.1.0]hexan-3-ylmethanol 146-3 (2.6 g, 57%) as a yellow oil. GC-MS: m/z 114 (5) (M⁺), 95 (15), 88 (100), 81 (15).

### Step 4. Preparation of 2-amino-4-(hydroxymethyl)cyclopentan-1-ol 146-4

To a solution of 6-oxabicyclo[3.1.0]hexan-3-ylmethanol 146-3 (2.0 g. 17.6 mmol) in methanol (20 mL) at 0° C. was purged ammonia gas for 10 min. The reaction was stirred at room temperature overnight. TLC indicated the incompletion of the reaction. Methanol was removed and NH₃H₂O (50 mL) was added and this was stirred at room temperature over a week. TLC confirmed the completion of the reaction. Water was removed by azeotropically with ethanol to afford 2-amino-4-(hydroxymethyl)cyclopentanol 146-4 (2.1 g, 91%) as a yellow oil.

### Step 5. Preparation of Methyl 12-(2-hydroxy-4-(hydroxymethyl)cyclopentylamino)-12-oxododecanoate 146-5

Compound 146-5 was prepared from 2-amino-4-(hydroxymethyl)cyclopentanol 146-4 and 1-(2,5-dioxopyrrolidin-1-yl) 12-methyl dodecanedioate 146-1, using the same procedure as described in the synthesis of 12-(2-(tert-butoxycarbonylamino)ethylamino)-12-oxododecanoate (3-2). Methyl 12-(2-hydroxy-4-(hydroxymethyl)cyclopentylamino)-12-oxododecanoate 146-5 was obtained in 87.4% yield as an off-white solid.

### Step 6. Preparation of Compound 147

Compound 147 was prepared quantitatively from compound 146 (1.4 g, 2.33 mmol) using an identical procedure to that used for compound 18.

### Step 7. Preparation of Compound 148

Compound 148 was prepared from compound 147 (150 mg, 0.23 mmol) and compound 14 (431 mg, 0.23 mmol) using an identical procedure to that used for compound 19. Yield: 460 mg, 84%.

### Step 8. Preparation of Compound 149

Compound 149 was prepared from compound 148 (460 mg, 0.19 mmol) using an identical procedure to that used for compound 20. Yield: 436 mg, 91%.

### Step 9. Preparation of Conjugate 150

Compound 150 was prepared from compound 149 (436 mg) and 1000 A lcaa CPG (2.62 g) using an identical procedure to that used for compound 1. Yield: 2.7 g, 21.31 μmol/g CPG loading. The resulting GalNAc loaded CPG solid support was employed in automated oligonucleotide synthesis using standard procedures. Nucleotide deprotection followed by removal from the solid support (with concurrent galactosamine acetate deprotection) afforded the GalNAc-oligonucleotide conjugate 150.

### Example 11. Synthesis of Conjugates 153, 158, 163, 168 and 173

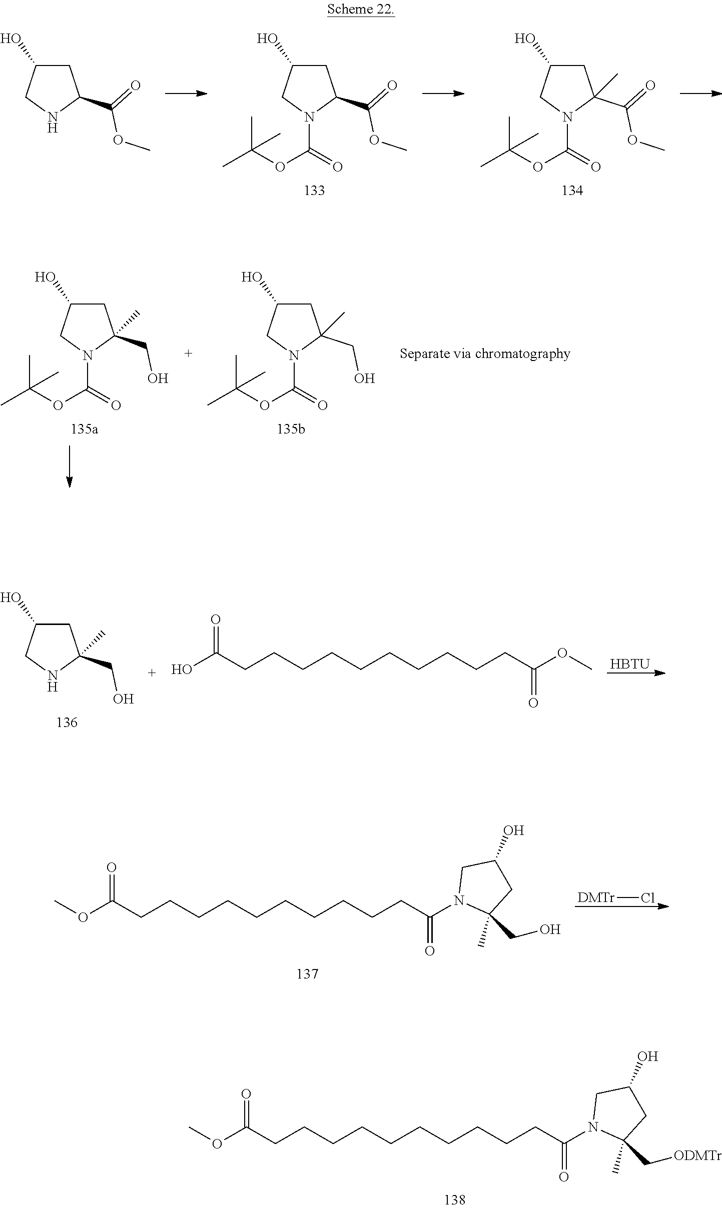

### Step 1. Preparation of 1-(tert-butyl) 2-methyl (2S,4R)-4-hydroxypyrrolidine-1,2-dicarboxylate (133)

Methyl (2S,4R)-4-hydroxypyrrolidine-2-carboxylate (25.9 g, 46 mmol), BOC anhydride (65.9 g, 302.5 mmol) and TEA (42 ml, 302.5 mmol) were stirred in DCM at RT for 16 h. The organics were washed sequentially with 1M HCl (×2), saturated NaHCO₃ (×2), H₂O and brine, dried and concentrated in-vacuo to give 1-(tert-butyl) 2-methyl (2S,4R)-4-hydroxypyrrolidine-1,2-dicarboxylate (133) (58.1 g, 85%).

### Step 2. Preparation of 1-(tert-butyl) 2-methyl (4R)-4-hydroxy-2-methylpyrrolidine-1,2-dicarboxylate (134)

1-(tert-butyl) 2-methyl (2S,4R)-4-hydroxypyrrolidine-1,2-dicarboxylate (133) (5 g, 20.4 mmol) and MeI (12 g, 84.5 mmol) were stirred in anhydrous THF at −40° C. LDA (2.0 M solution in THF) (37.5 mL, 75 mmol) was added dropwise. The reaction was allowed to warm to RT and stirred for 4 h then quenched with saturated NH₄Cl. The reaction was extracted with EtOAc, washed with H₂O and brine, dried (Na₂SO₄) and concentrated in-vacuo. The residue was purified by column chromatography 50:50 EtOAc//hexanes to give 1-(tert-butyl) 2-methyl (4R)-4-hydroxy-2-methylpyrrolidine-1,2-dicarboxylate (134) as a racemic mixture (3.6 g, 68%)

### Step 3. Preparation of tert-butyl (2S,4R)-4-hydroxy-2-(hydroxymethyl)-2-methylpyrrolidine-1-carboxylate (135a)

1-(Tert-butyl) 2-methyl (4R)-4-hydroxy-2-methylpyrrolidine-1,2-dicarboxylate (134) (19 g, 73.5 mmol) was stirred in anhydrous THF under N₂. LiBH₄ solution (48 ml, 96 mmol) was added dropwise and the reaction stirred at RT for 48 h. The reaction was quenched with 1M NaOH, the THF removed in-vacuo and the residual extracted with EtOAc (4×100 ml). The organics were washed with H₂O and brine, dried (Na₂SO₄) and concentrated in-vacuo. The residue was purified by column chromatography (5% MeOH/DCM) to give tert-butyl (2S,4R)-4-hydroxy-2-(hydroxymethyl)-2-methylpyrrolidine-1-carboxylate (135a) as the major product (8 g, 47%). Structure assigned according to literature references.

### Step 4. Preparation of (3R,5S)-5-(hydroxymethyl)-5-methylpyrrolidin-3-ol hydrochloride (136)

tert-Butyl (2S,4R)-4-hydroxy-2-(hydroxymethyl)-2-methylpyrrolidine-1-carboxylate (135a) (8 g, 34.6 mmol) was stirred in EtOAc at RT and gaseous HCl applied for approximately two minutes. The reaction was stirred for one hour then concentrated in-vacuo and dried under high vacuum to give (3R,5S)-5-(hydroxymethyl)-5-methylpyrrolidin-3-ol hydrochloride (136) in quantitative fashion.

### Step 5. Preparation of methyl 12-((2S,4R)-4-hydroxy-2-(hydroxymethyl)-2-methylpyrrolidin-1-yl)-12-oxododecanoate (137)

(3R,5S)-5-(Hydroxymethyl)-5-methylpyrrolidin-3-ol hydrochloride (136) (7.9 g, 47.4 mmol), 12-methoxy-12-oxododecanoic acid (11.5 g, 47.4 mmol), HBTU (36 g, 76 mmol) and TEA 20 mL, 142.2 mmol) were stirred in DCM at RT for 16h. The precipitate was removed by filtration and the organics washed with 1M HCl (×2), saturated NaHCO₃ (×2), H₂O and brine. After drying the organics were concentrated in-vacuo and purified by column chromatography (5% MeOH/DCM) to give methyl 12-((2S,4R)-4-hydroxy-2-(hydroxymethyl)-2-methylpyrrolidin-1-yl)-12-oxododecanoate (137) (3.1 g, 18.3%).

### Step 6. Preparation of methyl 12-((2S,4R)-2-((bis(4-methoxyphenyl)(phenyl)methoxy)-methyl)-4-hydroxy-2-methylpyrrolidin-1-yl)-12-oxododecanoate (138)

Methyl 12-((2S,4R)-4-hydroxy-2-(hydroxymethyl)-2-methylpyrrolidin-1-yl)-12-oxododecanoate (137) (3.1 g, 9.0 mmol), DMTr-Cl (2.8 g, 8.2 mmol) and TEA (1.1 ml, 8.2 mmol) were stirred in DC<at RT for 16 h. The reaction was concentrated in-vacuo and the residue purified by column chromatography (5% MeOH/DCM, 0.1% TEA) to give methyl 12-((2S,4R)-2-((bis(4-methoxyphenyl)(phenyl)methoxy)methyl)-4-hydroxy-2-methylpyrrolidin-1-yl)-12-oxododecanoate (138) (2.7 g, 45.5 mmol).

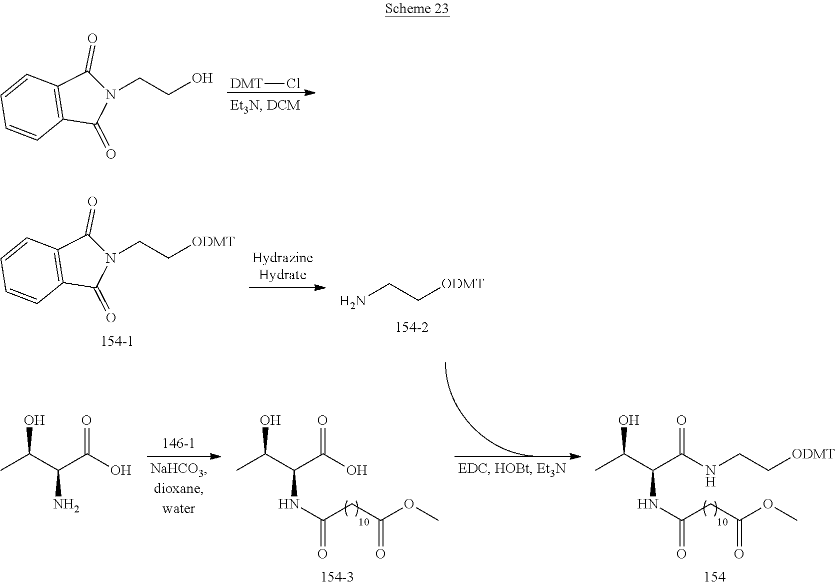

### Step 7. Preparation of Compound 154-1

To a solution of N-(2-hydroxyethyl)phthalimide (4.80 g, 25.0 mmol) and 4,4′-dimethoxytrityl chloride (8.8 g, 26.0 mmol) in dichloromethane (200 mL) at 0° C. under nitrogen, was added triethylamine (10.4 mL, 74.6 mmol) dropwise. The reaction mixture was stirred at room temperature for 3 hrs. TLC indicated the completion of the reaction. The organic layer was washed with brine (100 mL), dried over MgSO4, and concentrated to dryness. This was used directly for the next reaction without purification.

### Step 8. Preparation of Compound 154-2

2-(2-(Bis(4-methoxyphenyl)(phenyl)methoxy)ethyl)isoindoline-1,3-dione (154-1) obtained above and hydrazine monohydrate (3.6 mL, 74 mmol) in ethanol (100 mL) was stirred overnight at room temperature. TLC indicated the completion of the reaction. The precipitate was filtered out. The filtrate was evaporated. The residue was taken up by ethyl acetate (100 mL). The organic solution was washed with 10% NaOH, water and brine, and dried over MgSO4. Evaporation of solvent afforded 2-(bis(4-methoxyphenyl)(phenyl)methoxy)ethanamine (154-2) as a yellow liquid (8.11 g, 89.3% yield over two steps). This was used for the next reaction without further purification.

### Step 9. Preparation of Compound 154-3

To a solution of L-threonine (1.19 g, 10.0 mmol) and NaHCO₃ (2.3 g, 27 mmol) in water (20 mL) and dioxane (10 mL), was added 1-(2,5-dioxopyrrolidin-1-yl) 12-methyl dodecanedioate 146-1 (3.1 g, 9.1 mmol) in dioxane (10 mL) dropwise. The reaction mixture was stirred at room temperature overnight. 4N HCl (10 mL) was added. The precipitate was collected by filtration and washed with water (3×10 mL). The solid was dried over P20s in a desiccator to afford (2S,3R)-3-hydroxy-2-(12-methoxy-12-oxododecanamido)butanoic acid 154-3 as an off-white solid (2.84 g, 82.2%). LC-MS (ESI): m/z: 346 (100), (M+H⁺).

### Step 10. Preparation of Compound 154

(2S,3R)-3-hydroxy-2-(12-methoxy-12-oxododecanamido)butanoic acid 154-3 (2.47 g, 7.15 mmol), 2-(bis(4-methoxyphenyl)(phenyl)methoxy)ethanamine 154-2 (2.60 g, 7.15 mmol), EDC (1.64 g, 8.58 mmol), 1-hydroxybenzotriazole (HOBt) (1.16 g, 8.58 mmol) and TEA (2.4 mL, 17.2 mmol) were stirred in dichloromethane (72 mL) at room temperature for 2 hrs. Water (30 mL) was added. The organic layer was separated and washed with brine (2×30 mL). Evaporation of solvent followed by column chromatography (30% ethyl acetate/hexanes −50% ethyl acetate/hexanes) afforded methyl 12-((2S,3R)-1-(2-(bis(4-methoxyphenyl)(phenyl)methoxy)ethylamino)-3-hydroxy-1-oxobutan-2-ylamino)-12-oxododecanoate 154 as a waxy yellow semi-solid (2.60 g, 52.6%). ¹HNMR (400 MHz, acetone-d6, ppm): δ 7.51 (t, J=5.5 Hz, 1H), 7.45-7.49 (m, 2H), 7.28-7.36 (m, 6H), 7.21 (tt, J=7.2, 1.2 Hz, 1H), 7.08 (d, J=8.1 Hz, 1H), 6.88 (dt, J=8.9, 2.5 Hz, 4H), 4.39 (dd, J=8.2, 3.0 Hz, 1H), 4.20-4.27 (m, 1H), 3.78 (s, 6H), 3.60 (s, 1H), 3.35-3.52 (m, 2H), 3.07-3.16 (m, 2H), 2.23-2.37 (m, 4H), 1.53-1.65 (m, 4H), 1.23-1.36 (m, 12H), 1.10 (d, J=6.4 Hz, 3H).

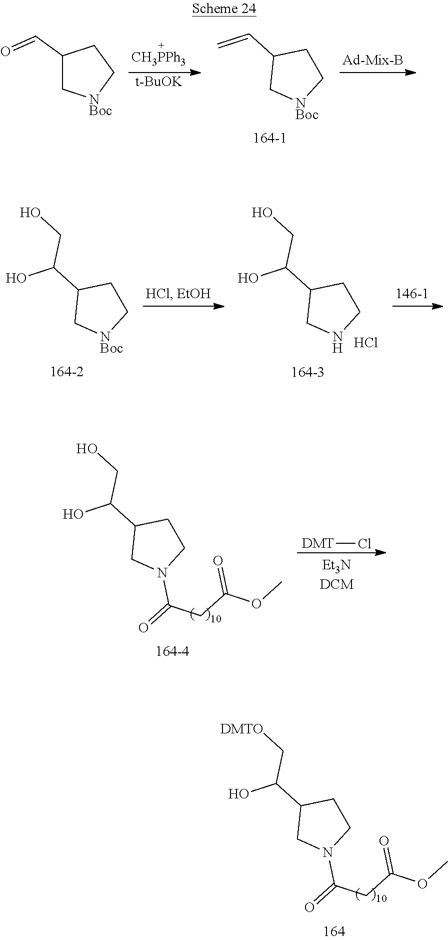

### Step 11. Preparation of Compound 164-1

To a suspension of potassium t-butoxide (14.6 g, 130 mol) in THF (120 mL)/ether (360 mL) was added methyltriphenylphosphonium bromide (46.6 g, 130 mmol). The mixture was refluxed for 2 hrs and then cooled to 0° C. tert-butyl 2-formylpyrrolidine-1-carboxylate (13.0 g, 65.2 mmol) in ether (50 mL) was added dropwise. The reaction mixture was stirred at 0° C. and then quenched by the addition of water (250 mL). The organic layer was separated and the aqueous was extracted with ether (250 mL). The combined extract was dried over MgSO4. Evaporation of solvent, followed by column chromatography purification (5% ethyl acetate/hexanes) gave tert-butyl 3-vinylpyrrolidine-1-carboxylate 164-1 (11.5 g, 89.4%) as a colorless liquid. GC-MS: m/z: 197 (2) (M⁺), 141 (40), 124 (30), 57 (100).

### Step 12. Preparation of Compound 164-2

To a mixture of t-BuOH (140 mL) and water (70 mL), was charged AD-mix-β (47.4 g) and methanesulfonamide (2.89 g, 30.4 mmol). The mixture was stirred at room temperature for 30 min and was then cooled to 0° C. tert-Butyl 3-vinylpyrrolidine-1-carboxylate 164-1 (6.00 g, 30.4 mmol) was added. The reaction was stirred at room temperature overnight. The reaction mixture was cooled to 0° C. Sodium thiosulfate pentahydrate (96 g, 387 mmol) was added and the temperature was allowed to warm to room temperature. Water (700 mL) was added and the mixture was extracted with ethyl acetate (500 mL). The extract was washed with water (2×50 mL) and brine (50 mL), and dried over MgSO₄. Evaporation of solvent, followed by column chromatography (2% methanol/dichloromethane-7% methanol/dichloromethane) gave tert-butyl 3-(1,2-dihydroxyethyl)pyrrolidine-1-carboxylate 164-2 (5.4 g, 77%) as a light brown oil.

### Step 13. Preparation of Compound 164-3

To a solution of tert-butyl 3-(1,2-dihydroxyethyl)pyrrolidine-1-carboxylate 164-2 (3.1 g, 13.4 mmol) in ethanol (10 mL) was added 3N HCl (30 mL, 90 mmol). The reaction mixture was stirred at room temperature overnight. TLC indicated the completion of the reaction. Ethanol was evaporated. Toluene was added and evaporated. This was repeated three times to give 1-(pyrrolidin-3-yl)ethane-1,2-diol hydrochloride 164-3 (2.0 g, 89%) as a brown oil. LC-MS (ESI): m/z: 132 (100), (M+H⁺, free amine).

### Step 14 Preparation of Compound 164-4

To a solution of 1-(pyrrolidin-3-yl)ethane-1,2-diol hydrochloride 164-2 (2.0 g, 12 mmol) in water (30 mL) was added NaHCO₃ (3.7 g, 44 mmol) by portion. Dioxane (20 mL) was then added. To the above solution was added 1-(2,5-dioxopyrrolidin-1-yl) 12-methyl dodecanedioate 146-1 (3.7 g, 11 mmol) in dioxane (30 mL). The reaction mixture was stirred overnight. This was extracted with ethyl acetate (3×100 mL). The combined extract was washed with 0.5N HCl (50 mL) and brine (50 mL), and dried over MgSO4.

### Step 15. Preparation of Compound 164

This substance was prepared from methyl 12-(3-(1,2-dihydroxyethyl)pyrrolidin-1-yl)-12-oxododecanoate 164-4 and 4,4-dimethoxytrityl chloride (1 eq) using the same procedure as described in the synthesis of 2-(2-(bis(4-methoxyphenyl)(phenyl)methoxy)ethyl)isoindoline-1,3-dione 138. The product was purified by column chromatography (1.5% methanol/dichloromethane). Methyl 12-(3-(2-(bis(4-methoxyphenyl)(phenyl)methoxy)-1-hydroxyethyl)pyrrolidin-1-yl)-12-oxododecanoate 164 was obtained in 51% yield as a yellow oil. ¹HNMR (400 MHz, acetone-d6, ppm): δ 7.49-7.54 (m, 2H), 7.35-7.40 (m, 4H), 7.28-7.34 (m, 2H), 7.19-7.25 (m, 1H), 6.86-6.91 (m, 4H), 4.11-4.20 (m, 1H), 3.79 (s, 6H), 3.68-3.77 (m, 1H), 3.60 (s, 3H), 3.29-3.59 (m, 3H), 3.06-3.20 (m, 3H), 2.33-2.55 (m, 1H), 2.29 (t, J=7.4 Hz, 2H), 2.19 (t, J=7.6 Hz, 2H), 1.65-2.0 (m, 2H), 1.51-1.62 (m, 4H), 1.26-1.35 (m, 12H).

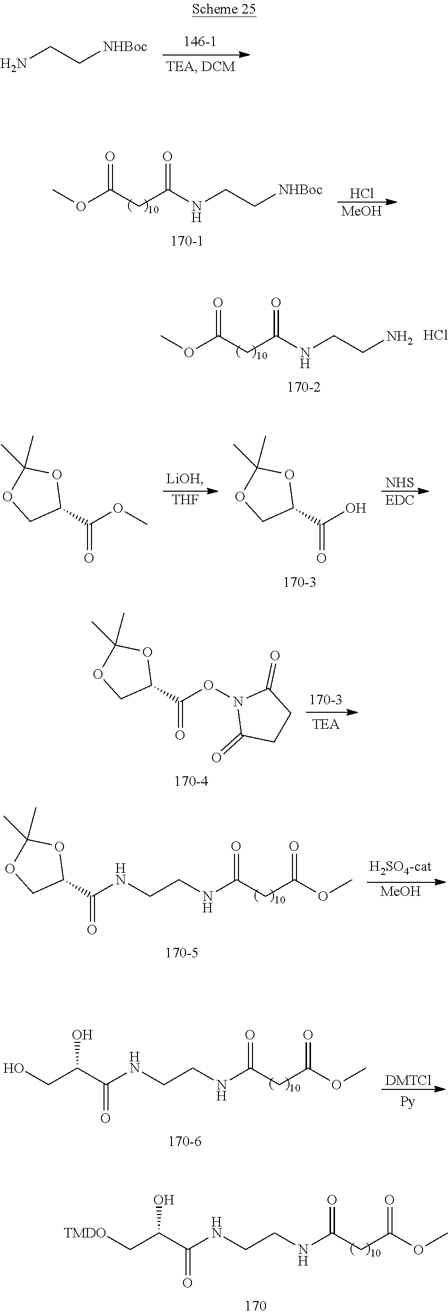

### Step 16. Preparation of Compound 170-1

To a solution of tert-butyl 2-aminoethylcarbamate (2.88 g, 18.0 mmol) and triethylamine (2.98 g, 29.4 mmol) in dichloromethane (100 mL), was added 1-(2,5-dioxopyrrolidin-1-yl) 12-methyl dodecanedioate (146-1) (5.09 g, 14.9 mmol) in dichloromethane (50 mL) dropwise at room temperature. The reaction mixture was stirred overnight and TLC indicated the completion of the reaction. 100 mL brine was added and the organic layer was separated. The organic layer was washed with 0.5N HCl (150 mL), brine (2×100 mL) and dried over MgSO₄. Evaporation of solvent gave pure methyl 12-(2-(tert-butoxycarbonylamino)ethylamino)-12-oxododecanoate 170-1 (5.85 g 100%) as a white solid.

### Step 17. Preparation of Compound 170-2

To a solution of 12-(2-(tert-butoxycarbonylamino)ethylamino)-12-oxododecanoate 170-1 (5.55 g, 14.4 mmol) in methanol (100 mL) at 0° C., was added thionyl chloride (3.3 mL, 45.5 mmol) dropwise. The reaction was then stirred at room temperature overnight. TLC indicated the completion of the reaction. The solvent and volatile organics were evaporated. The residue was then co-evaporated with heptanes twice to give methyl 12-(2-aminoethylamino)-12-oxododecanoate hydrochloride 170-2 quantitatively as a white solid. LC-MS (ESI): m/z: 287 (100), (M+H⁺, free amine).

### Step 18. Preparation of Compound 170-3

(−)-Methyl (S)-2,2-dimethyl-1,3-dioxolane-4-carboxylate (5.01 g, 31.2 mmol) and LiOH.H₂O (2.55 g, 60.8 mmol) in THF (50 mL) and water (50 mL) was stirred overnight. TLC indicated the completion of the reaction. THF was evaporated and the aqueous was acidified with 1N HCl to pH=1. This was extracted with ethyl acetate (5×50 mL). The combined extract was dried over MgSO4. Evaporation of solvent gave (S)-2,2-dimethyl-1,3-dioxolane-4-carboxylic acid 170-3 (2.93 g, 64.3%) as a light yellow liquid.

### Step 19. Preparation of Compound 170-4

Compound 170-4 was synthesized from (S)-2,2-dimethyl-1,3-dioxolane-4-carboxylic acid 170-3 and N-hydroxysuccinimide in 86% yield, using the same procedure as described in the synthesis of 1-(2,5-dioxopyrrolidin-1-yl) 12-methyl dodecanedioate 146-1. (S)-2,5-Dioxopyrrolidin-1-yl 2,2-dimethyl-1,3-dioxolane-4-carboxylate 170-4 was obtained in 86% yield as a white solid.

### Step 20. Preparation of Compound 170-5

To a suspension of methyl 12-(2-aminoethylamino)-12-oxododecanoate hydrochloride 170-2 (14.4 mmol) and (S)-2,5-dioxopyrrolidin-1-yl 2,2-dimethyl-1,3-dioxolane-4-carboxylate 170-4 (3.80 g, 15.6 mmol) in dichloromethane (100 mL) was added triethylamine (6 mL, 43.0 mmol) in dichloromethane (25 mL) over 4 hrs at 0° C. The reaction mixture was then stirred at room temperature overnight. LC-MS indicated that the starting material 170-2 was completely converted. The organic layer was washed with brine (50 mL), IN HCl (50 mL), brine (50 mL), dried over MgSO4 and concentrated to dryness to afford (S)-methyl 12-(2-(2,2-dimethyl-1,3-dioxolane-4-carboxamido)ethylamino)-12-oxododecanoate 170-5 (5.93 g, 99.3%) as a white solid.

### Step 21. Preparation of Compound 170-6

To a solution of (S)-methyl 12-(2-(2,2-dimethyl-1,3-dioxolane-4-carboxamido)ethylamino)-12-oxododecanoate 170-5 (5.93 g, 14.3 mmol) was added one drop of concentrated sulfuric acid. This was refluxed for 6 hrs and then cooled to room temperature. The solid was collected through filtration and washed twice with cold methanol. The solid was dried in the air (3.32 g). The second crop (0.42 g) was obtained from the mother liquid to give (S)-methyl 12-(2-(2,3-dihydroxypropanamido)ethylamino)-12-oxododecanoate 170-6 (3.74 g in total, 69.4%) as a white crystal. LC-MS (ESI): m/z: 375 (100), (M+H⁺). ¹HNMR (400 MHz, DMSO-d6, ppm): δ 7.79 (br, 2H), 5.49 (d, J=5.3 Hz, 1H), 4.66 (t, J=5.8 Hz, 1H), 3.83-3.88 (m, 1H), 3.55-3.61 (m, 4H), 3.41-3.47 (m, 1H), 3.05-3.15 (m, 4H), 2.29 (t, J=7.4 Hz, 2H), 2.03 (t, J=7.6 Hz, 2H), 1.42-1.52 (m, 4H), 1.18-1.29 (m, 12H).

### Step 22. Preparation of Compound 170

To a solution of (S)-methyl 12-(2-(2,3-dihydroxypropanamido)ethylamino)-12-oxododecanoate 170-6 (2.99 g, 7.99 mmol) in dry pyridine (57.5 mL) under nitrogen, was added 4,4′-dimethoxytrityl chloride (2.84 g, 8.38 mmol) in one portion. The reaction was stirred at room temperature for two days. Methanol (5 mL) was added to quench the reaction. Pyridine was evaporated. Toluene was added and then evaporated. This was repeated three times. Water (100 mL) was added and this was extracted with ethyl acetate (5×250 mL). The extracts were combined and dried over MgSO4. Evaporation of solvent, followed by column chromatography (1% methanol/dichloromethane-3% methanol/dichloromethane) gave (S)-methyl 12-(2-(3-(bis(4-methoxyphenyl)(phenyl)methoxy)-2-hydroxypropanamido)-ethylamino)-12-oxododecanoate 170 (1.70 g, 31.4%) as a viscous oil. ¹HNMR (400 MHz, acetone-d6, ppm): δ 7.64-7.70 (br, 1H), 7.47-7.51 (m, 2H), 7.33-7.37 (m, 4H), 7.26-7.32 (m, 2H), 7.20 (dt, J=7.3, 2.1 Hz, 1H), 7.11 (br, 1H), 6.86 (d, J=8.7 Hz, 4H), 4.84 (br, 1H), 4.21 (dd, J=5.1, 3.8 Hz, 1H), 3.78 (s, 6H), 3.60 (s, 1H), 3.25-3.42 (m, 6H), 2.28 (t, J=7.4 Hz, 2H), 1.48-1.62 (m, 4H), 1.21-1.34 (m, 12H).

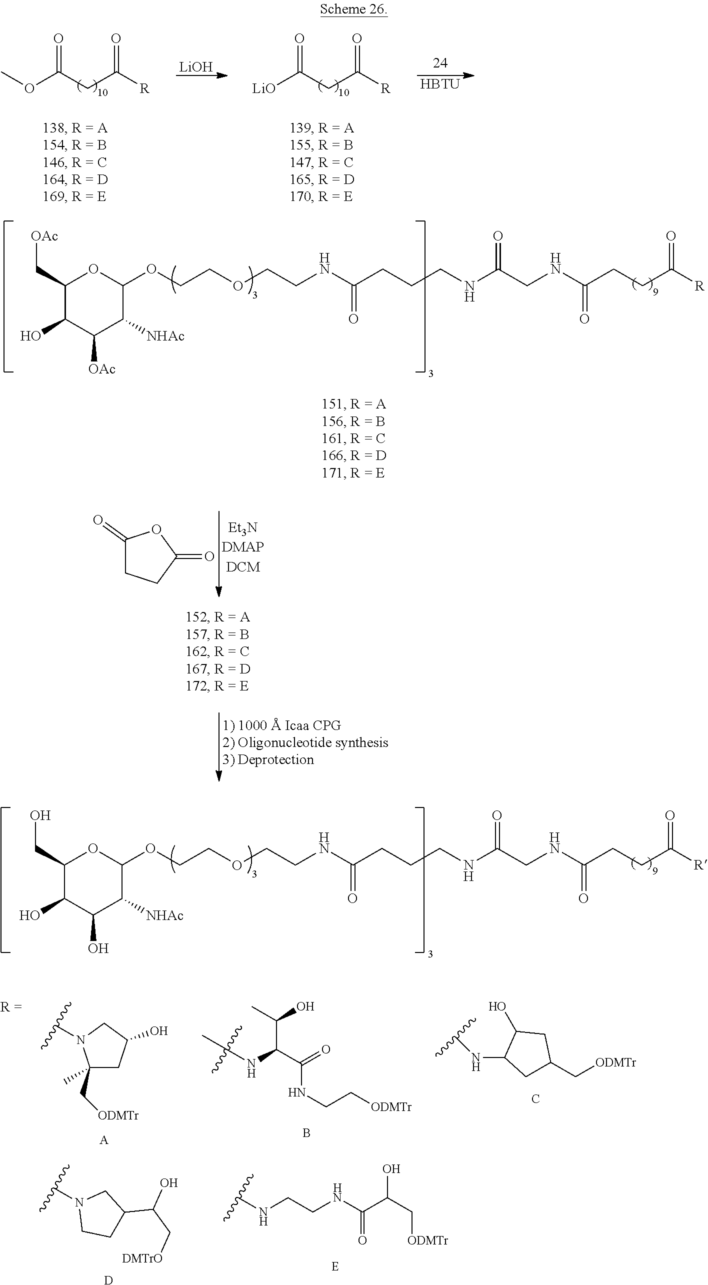

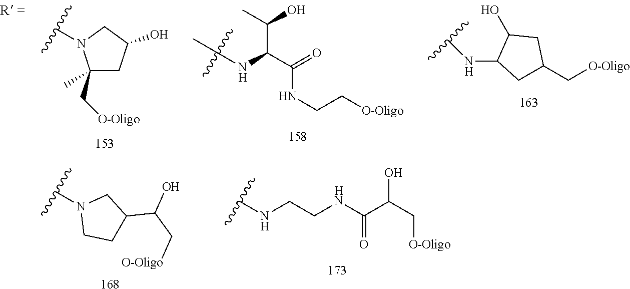

### Step 23. Preparation of Compounds 139, 155, 160, 165 and 170

Compounds 139, 155, 160, 165 and 170 were prepared from compounds 138, 154, 159, 164 and 169 using an identical procedure to that used for compound 18.

### Step 24. Preparation of Conjugates 153, 158, 163, 168 and 173

Conjugates 153, 158, 163, 168 and 173 were prepared from compound 139, 154, 159, 164 and 169 using an identical procedure to that used for compound 1.

### Example 12. Synthesis of Conjugate 176

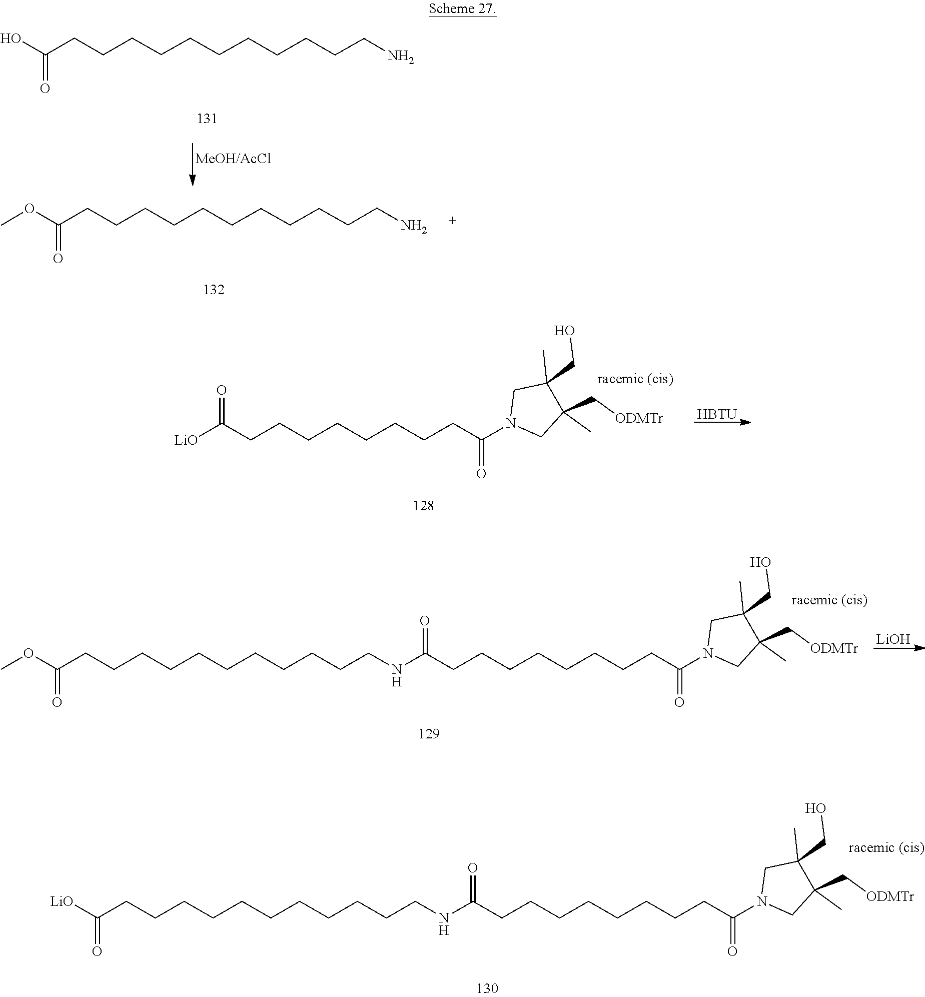

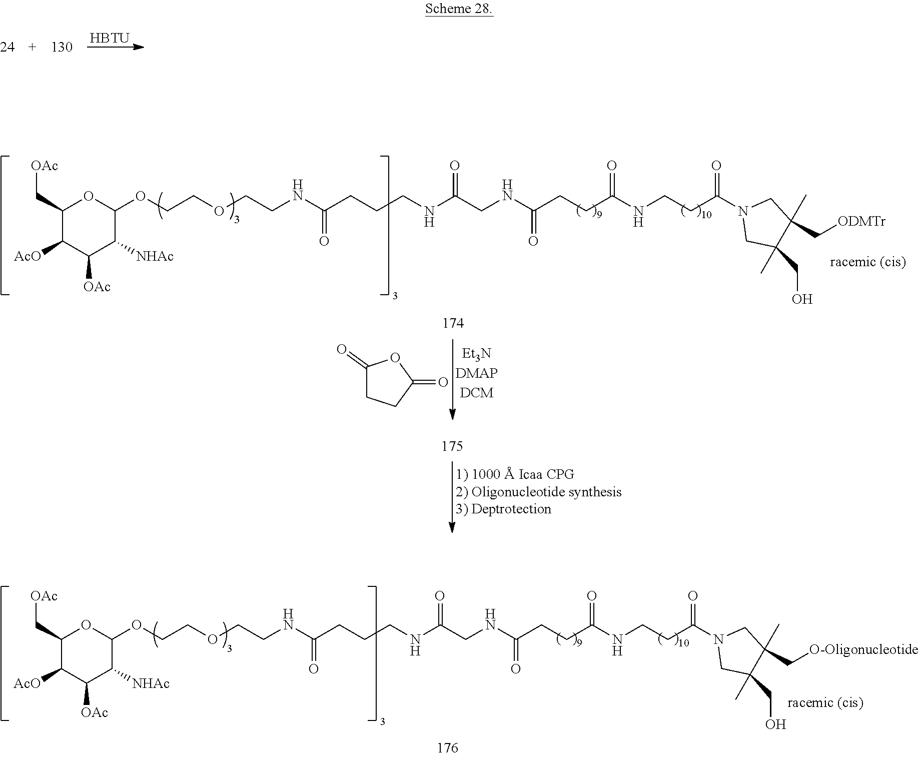

### Step. 1. Preparation of methyl 12-aminododecanoate 132

12-aminoundecanoic acid (131) (10 g, 4.64 mmol) was stirred in MeOH at RT. Acetyl chloride (856 μL, 12 mmol) was added dropwise and the reaction stirred for 1.5 hr. The solvent was removed in-vacuo, the residue taken up in MTBE and chilled in the fridge overnight. The resultant precipitate was collected by filtration, washed with ice cold MTBE and dried under high vacuum to afford methyl 12-aminododecanoate 132.

### Step 2. Preparation of Racemic (cis) Methyl 12-(12-(10-(3-((bis(4-methoxyphenyl)-(phenyl)methoxy)methyl)-4-(hydroxymethyl)-3,4-dimethylpyrrolidin-1-yl)-10-oxodecanamido)dodecanamido)dodecanoate 129

Lithium racemic (cis) 10-(3-((bis(4-methoxyphenyl)(phenyl)methoxy)methyl)-4-(hydroxymethyl)-3,4-dimethylpyrrolidin-1-yl)-10-oxodecanoate (128) (2 g, 3.1 mmol), of methyl 12-aminododecanoate (132) (778 mg, 3.1 mmol), HBTU (1.2 g, 3.1 mmol) and TEA (1.4 mL, 10 mmol) were stirred in DCM at RT O/N. The precipitate was removed by filtration, the filtrate concentrated in-vacuo and the residue purified by column chromatography (5% MeOH, DCM). TLC showed two close running spots with identical mass that were assigned as geometric isomers and pooled together to give of Methyl 12-(12-(10-((3R,4S)-3-((bis(4-methoxyphenyl)(phenyl)methoxy)methyl)-4-(hydroxymethyl)-3,4-dimethylpyrrolidin-1-yl)-10-oxodecanamido)dodecanamido)dodecanoate (129) in quantitative fashion.

### Step 3. Preparation of Racemic (cis) Lithium 12-(12-(10-(-3-((bis(4-methoxyphenyl)(phenyl)-methoxy)methyl)-4-(hydroxymethyl)-3,4-dimethylpyrrolidin-1-yl)-10-oxodecanamido)-dodecanamido)dodecanoate 130

Racemic (cis) methyl 12-(12-(10-(3-((bis(4-methoxyphenyl)(phenyl)methoxy)methyl)-4-(hydroxymethyl)-3,4-dimethylpyrrolidin-1-yl)-10-oxodecanamido)dodecanamido)dodecanoate (129) (3.1 mmol) was stirred in THF:H₂O (50:50) with LiOH (88 mg, 3.7 mmol) at RT O/N. Reaction was confirmed by TLC and the THF removed in-vacuo. The aqueous solution was frozen in liquid N₂ and lyophilized for 48 hours to give racemic (cis) Lithium 12-(12-(10-(3-((bis(4-methoxyphenyl)(phenyl)-methoxy)methyl)-4-(hydroxymethyl)-3,4-dimethylpyrrolidin-1-yl)-10-oxodecanamido)-dodecanamido)dodecanoate 130 quantitatively.

### Step 4. Preparation of Conjugate 176

Conjugate 176 was prepared from compounds 24 and 130 using an identical procedure to that used for compound 1.

### Example 13. Synthesis of Conjugate 179

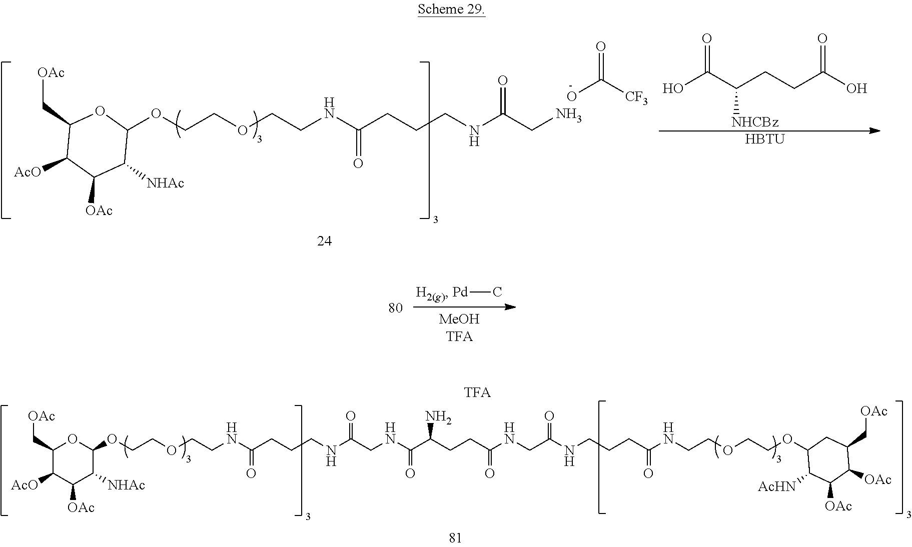

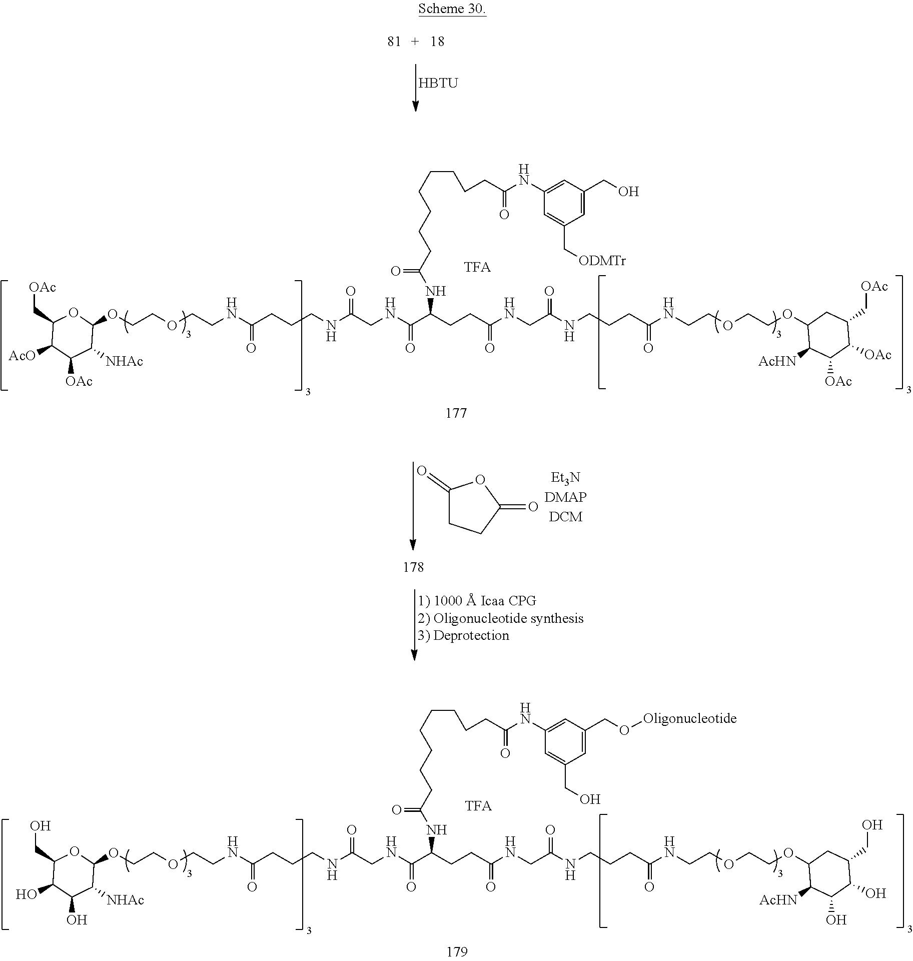

### Step 1. Preparation of Compound 80

Compound 24 (2 g, 0.86 mmol), N-carbobenzoxy-L-glutamic acid (120 mg, 0.43 mmol), HBTU (326 mg, 0.86 mmol) and TEA (353 μL, 2.6 mmol) were stirred in DCM at RT O/N. The mixture was concentrated in-vacuo and purified by column chromatography to give compound 80 (2.88 g, 83%).

### Step 2. Preparation of Compound 81

Compound 81 was prepared from compounds 80 (670 mg, 0.17 mmol) using an identical procedure to that used for compound 14. The compound was used crude in subsequent reactions and the yield taken as quantitative.

### Step 3. Preparation of Conjugate 179

Conjugate 179 was prepared from compounds 18 and 81 using an identical procedure to that used for compound 1.

### Example 14. Synthesis of Conjugate 182

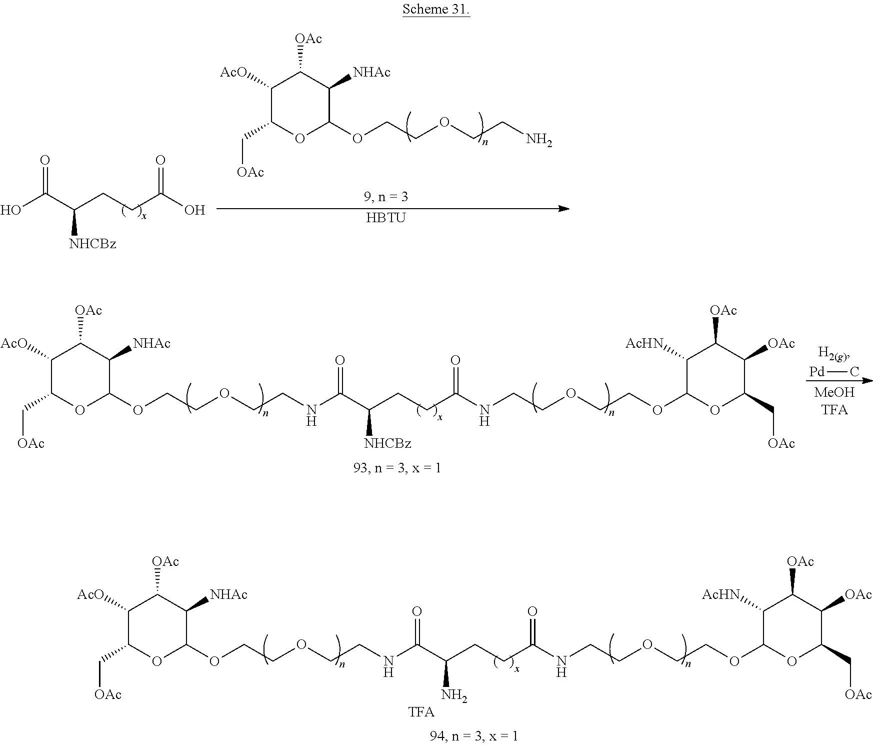

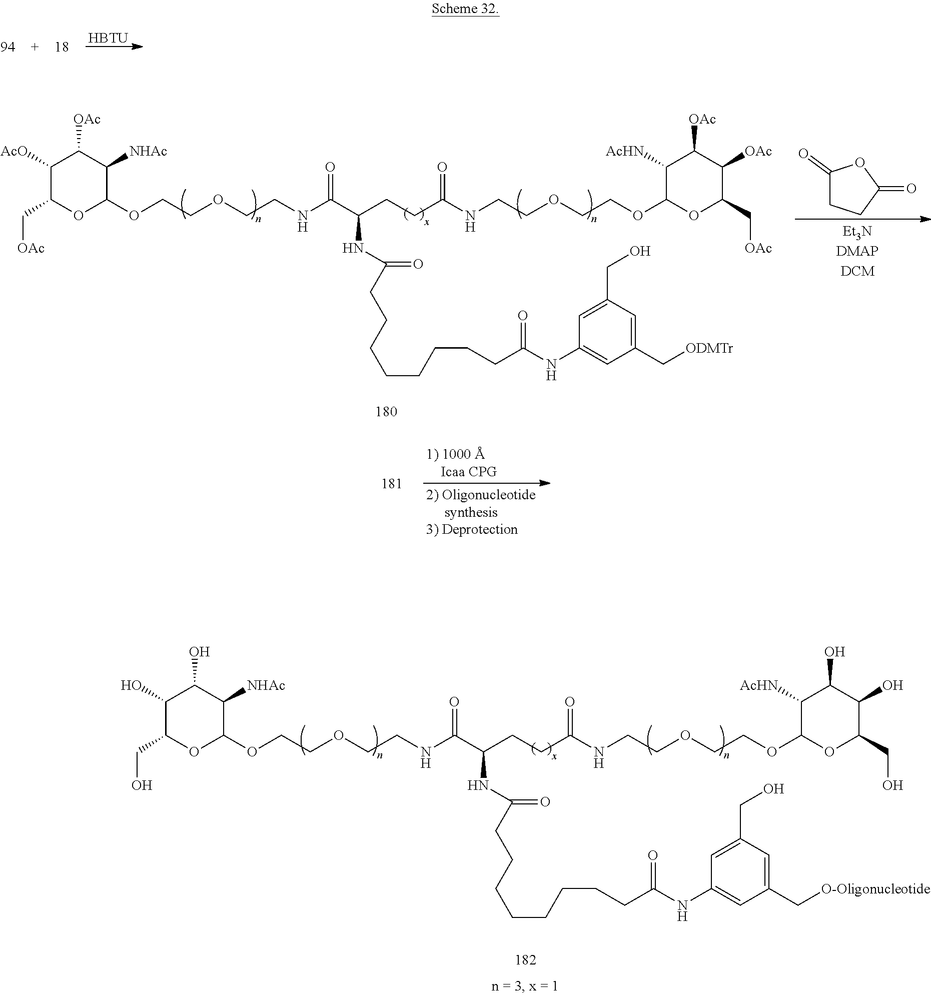

### Step 1. Preparation of Compound 93

Compound 93 was prepared from (2-oxo-2-phenyl-1λ²-ethyl)-D-glutamic acid (2.25 g, 8.1 mmol) and 9 (13 g, 21 mmol) using an identical procedure to that used for compound 89. Yield: 11.2 g.

### Step 2. Preparation of Compound 94

Compound 94 was prepared from compound 93 (11.1 g) using an identical procedure to that used for compound 90. Yield: 10.2 g.

### Step 3. Preparation of Conjugate 182

Conjugate 182 was prepared from compounds 18 and 94 using an identical procedure to that used for compound 1.

### Example 15. Synthesis of Conjugates 185 and 188

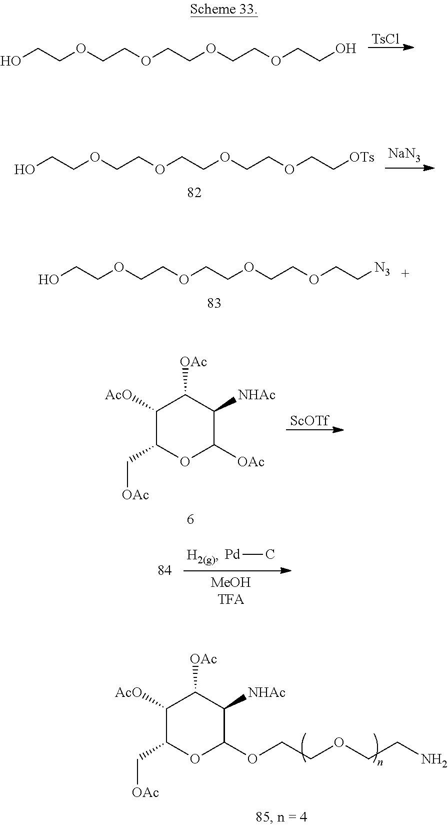

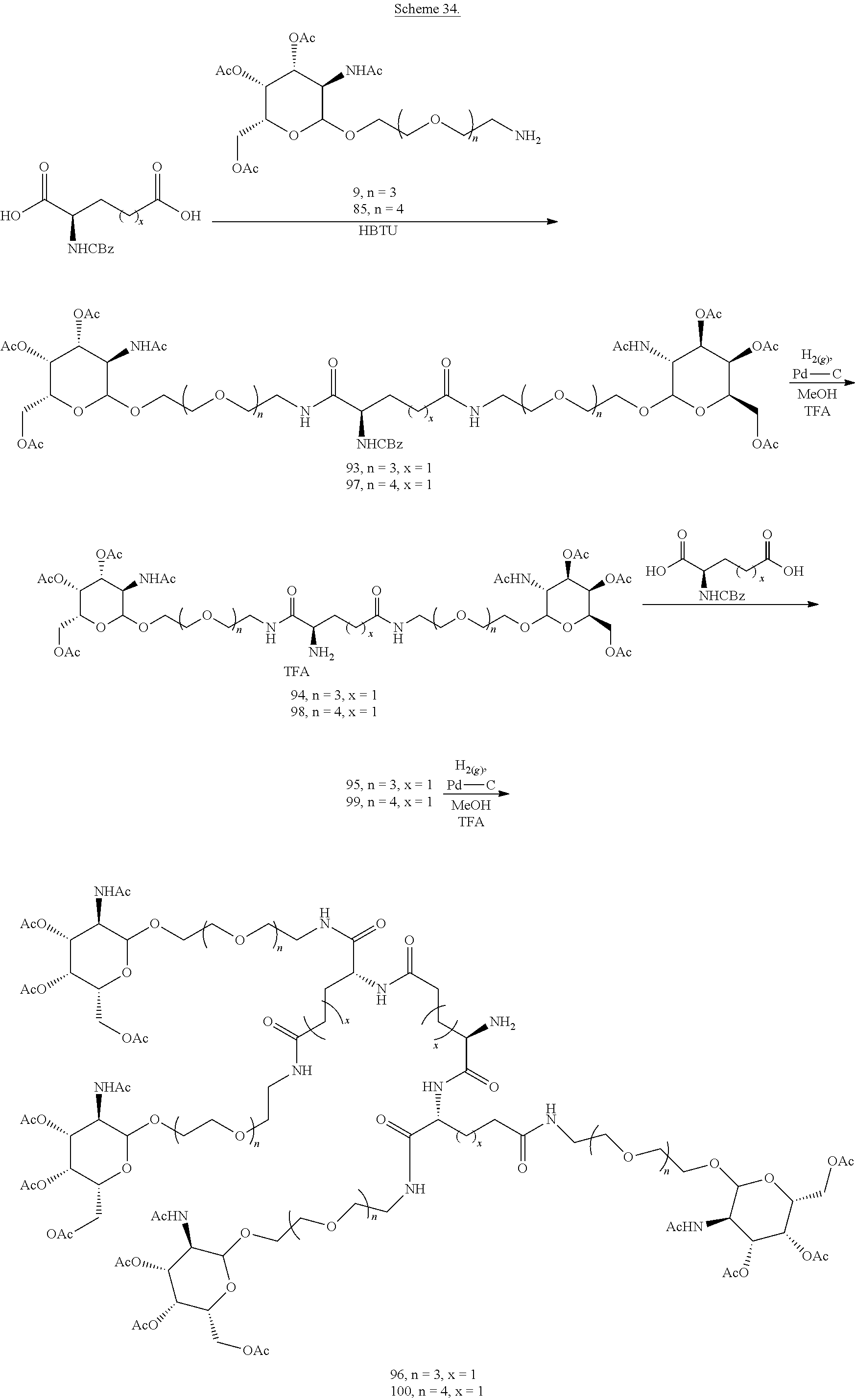

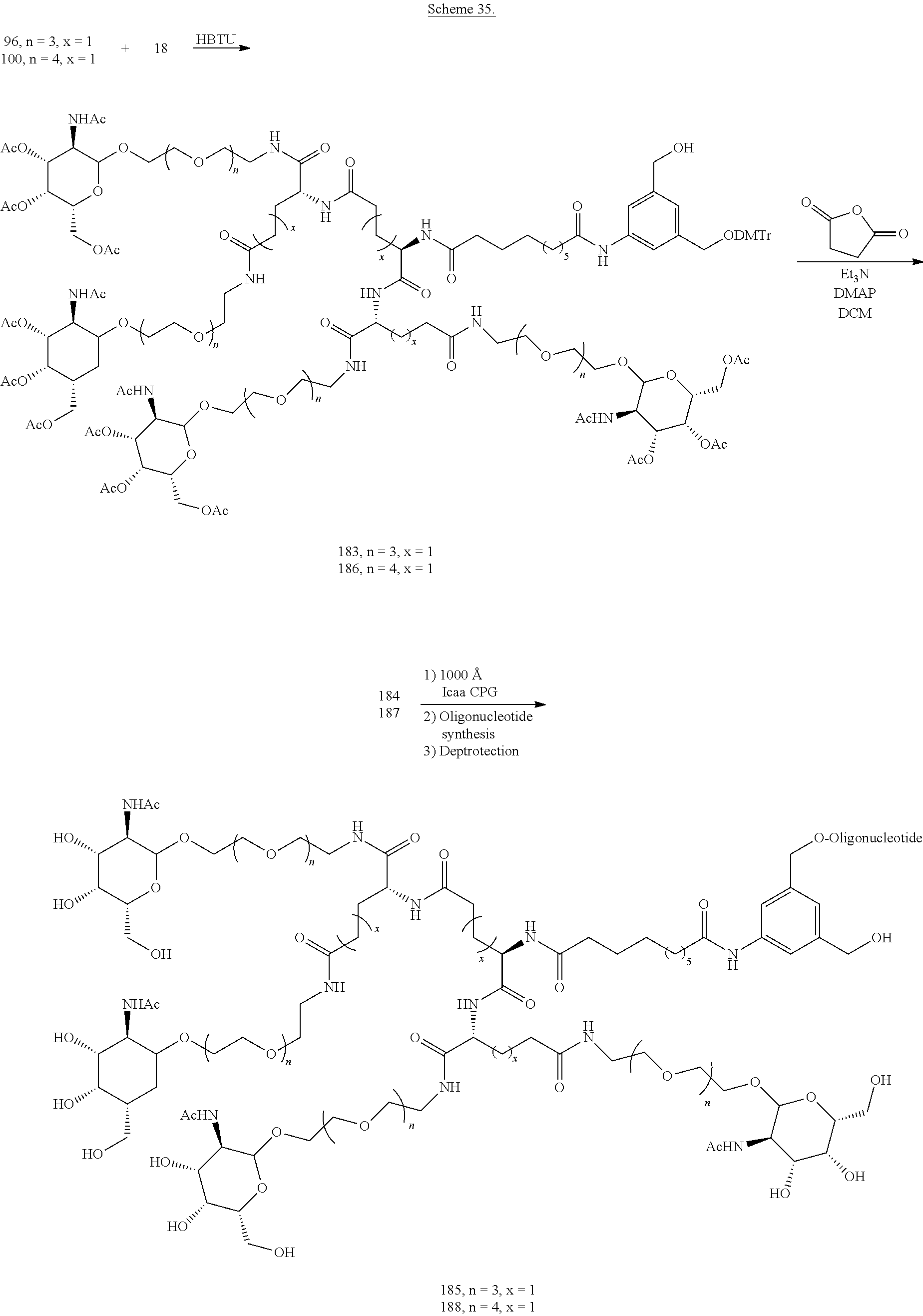

### Step 1. Preparation of 14-Hydroxy-3,6,9,12-tetraoxatetradecyl 4-methylbenzenesulfonate 82

A solution of pentaethylene glycol (35 g, 147 mmol), TEA (41 mL, 294 mmol) and trimethylamine-HCl (1.4 g, 14.7 mmol) in CH₂Cl₂ (600 mL) was treated with tosyl chloride (29.4 g, 154 mmol). After stirring (18h) the reaction mixture was washed with H₂O-brine (1:1), dried (MgSO4), filtered, concentrated and subjected to chromatography to yield 82 (24.6 g, 43%) as a pale yellow oil. Rf 0.8 (10/CH₃OH—CH₂Cl₂).

### Step 2. 14-azido-3,6,9,12-tetraoxatetradecan-1-oy 83

14-azido-3,6,9,12-tetraoxatetradecan-1-ol (83) was prepared from 82 (24.6 g. 62.7 mmol) and sodium azide (7.13 g. 110 mmol) using an identical procedure to that used for compound 4. Yield: 14.8 g. 90/o.

### Step 3. Preparation of Compound 84

A solution of GalNAc 6 (12.2 g, 31.4 mmol) and HO-PEG-N₃ 83 (9.2 g, 35 mmol) in 1,2-dichloroethane (150 mL) was treated with Sc(OTf)₃ (771 mg, 1.6 mmol). After stirring (85° C., 2 hr) the reaction was cooled (RT), quenched by the addition of TEA (40 mL) and concentrated. The crude material was subjected to chromatography to yield 84 (11.16 g, 60%) as a pale yellow foam. Rf 0.7 (10% CH₃OH—CH₂Cl₂).

### Step 4. Preparation of Compound 85

A solution of 84 (11.16 g, 18.8 mmol) and Pd/C (1.1 g, 10%—wet support) in EtOAc (120 mL) was treated with TFA (4.32 mL, 56.5 mmol) and purged with H₂. After stirring vigorously (4.5h) the reaction was purged with N₂, filtered through Celite and concentrated. The crude material was subjected to chromatography to yield 85 (5.77 g, 45%) as a colorless foam. Rf 0.5 (10% CH₃OH—CH₂Cl₂).

### Step 5. Preparation of Compound 95

Compound 95 was prepared from (2-oxo-2-phenyl-1λ²-ethyl)-D-glutamic acid (1.04 g, 3.7 mmol) and compound 94 (10.2 g) using an identical procedure to that used for compound 91. Yield: 7.2 g.

### Step 6. Preparation of Compound 96

Compound 96 was prepared from compound 95 (11.1 g) using an identical procedure to that used for compound 92. Yield: 6.5 g.

### Step 7. Preparation of Compound 97

Compound 97 was prepared from (2-oxo-2-phenyl-1λ²-ethyl)-D-glutamic acid (2 g, 7.1 mmol) and 85 (12.1 g, 17.8 mmol) using an identical procedure to that used for compound 89. Yield: 10 g, quantitative.

### Step 8. Preparation of Compound 98

Compound 98 was prepared from compound 97 (10 g, 7.2 mmol) using an identical procedure to that used for compound 90. Yield: 3.5 g, 36%.

### Step 9. Preparation of Compound 99

Compound 99 was prepared quantitatively from (2-oxo-2-phenyl-1λ²-ethyl)-D-glutamic acid (350 mg, 1.25 mmol) and compound 98 (2.86 mg, 2.5 mmol) using an identical procedure to that used for compound 91.

### Step 10. Preparation of Compound 100

Compound 100 was prepared quantitatively from compound 99 (3.2 g, 1.25 mmol) using an identical procedure to that used for compound 92.

### Step 11. Preparation of Conjugates 185 and 188

Conjugate 185 and 188 were prepared from compounds 18 and 96 or 18 and 100 using an identical procedure to that used for compound 1.

### Example 16. Synthesis of Conjugates 191, 194, 197 and 200

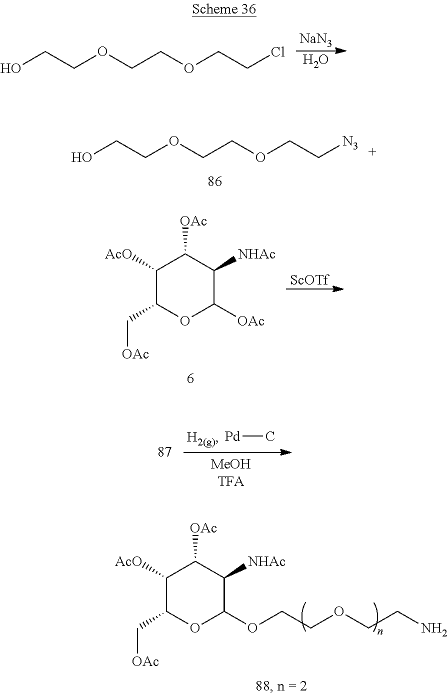

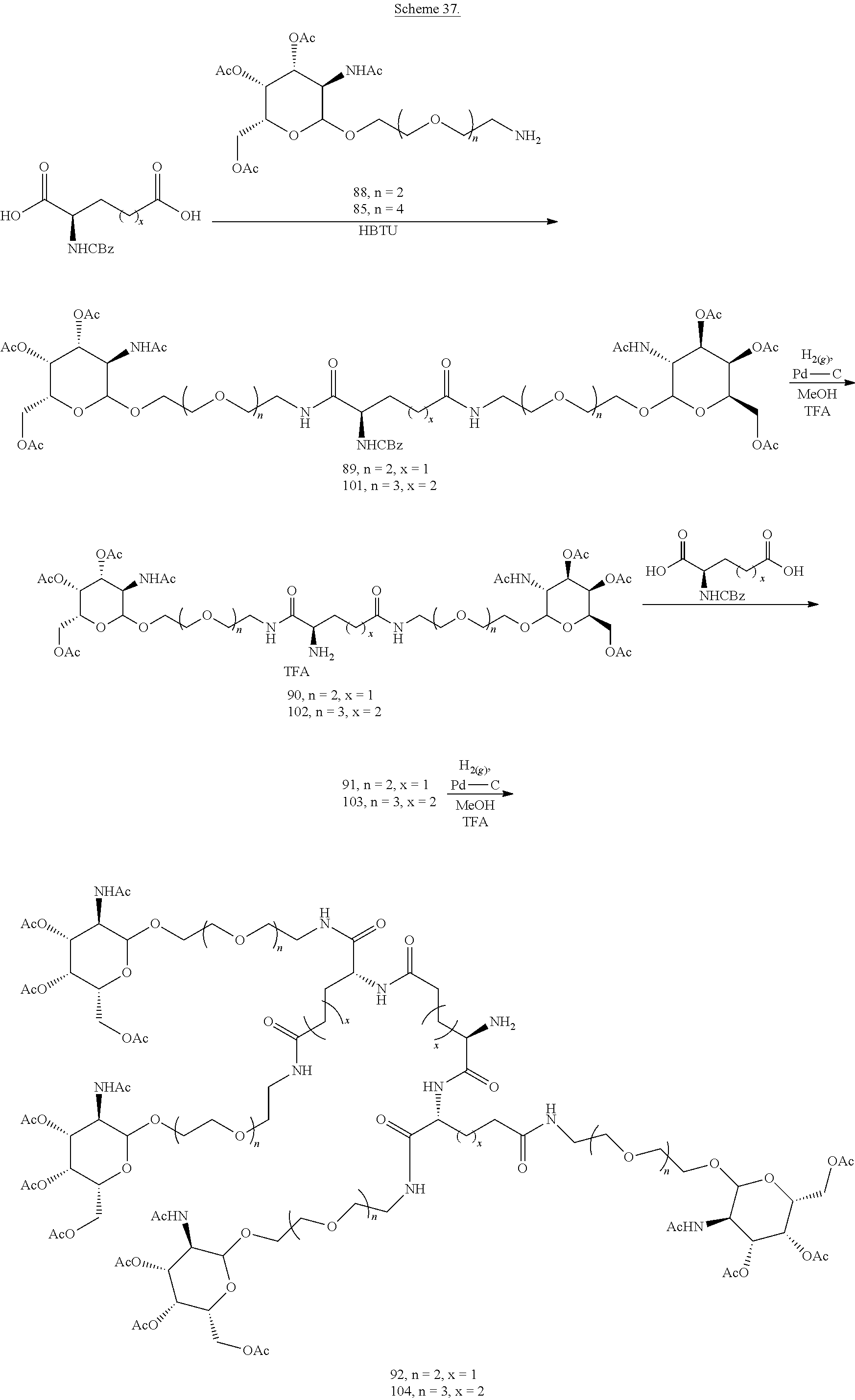

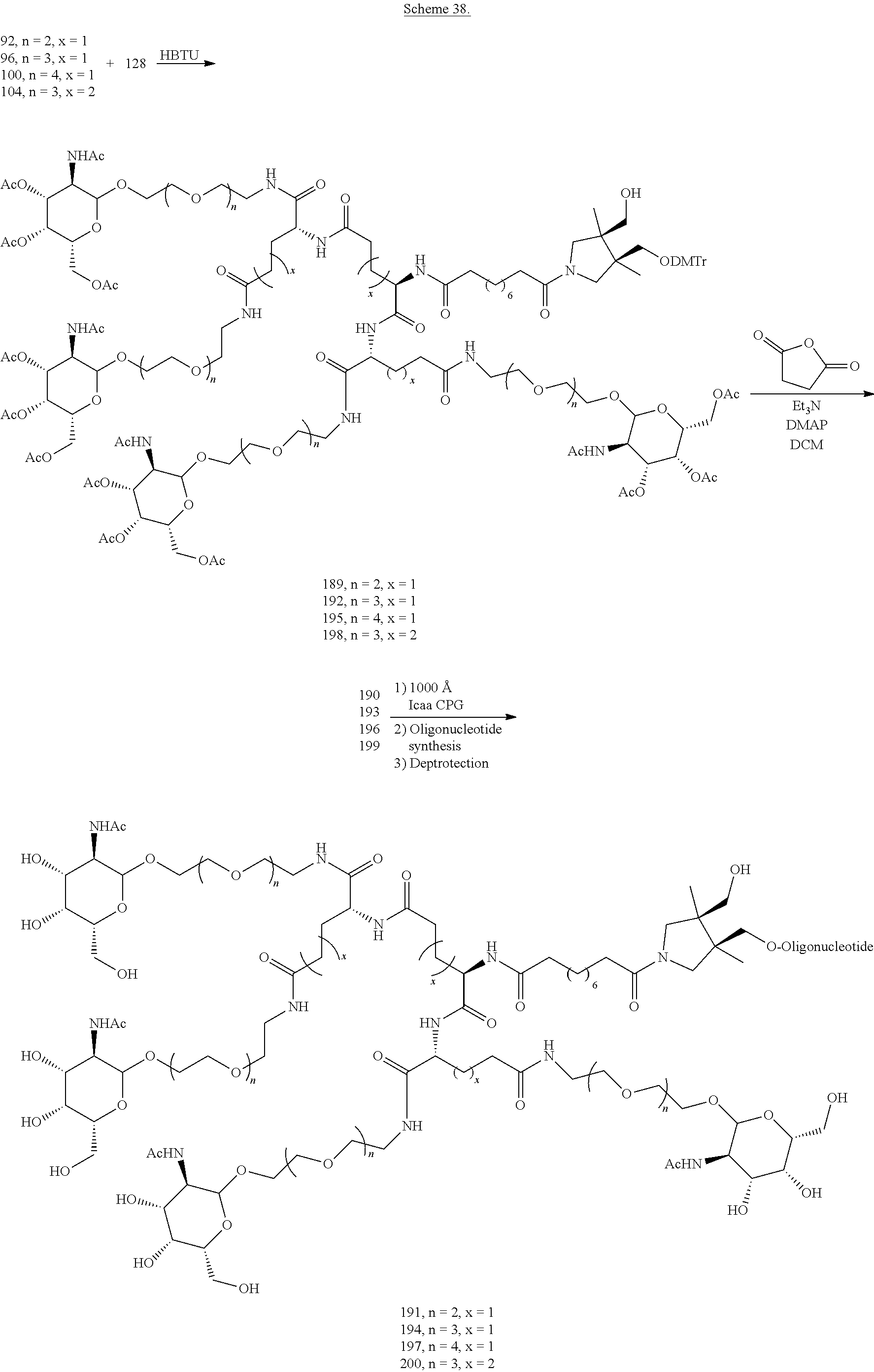

### Step 1. Preparation of 2-(2-(2-azidoethoxy)ethoxy)ethan-1-ol 86

To a solution of 2-(2-(2-chloroethoxy)ethoxy)ethan-1-ol (13 g, 77 mmol) in water (200 mL) is added sodium azide (10 g, 154 mmol). The reaction was heated to 100° C. for 18 hours. The reaction is cooled to room temperature and poured into a 1 L separatory funnel and extracted with dichloromethane (3×200 mL). The combine dichloromethane extracts are dried on magnesium sulfate, filtered and concentrated to dryness to afford 2-(2-(2-azidoethoxy)ethoxy)ethan-1-ol as a colorless oil (11.7 g).

### Step 2. Preparation of Compound 87

Compound 87 is prepared from 86 (4.95 g, 28.3 mmol) and 6 (10 g, 25.7 mmol) using an identical procedure to that used for compound 84. Yield: 10 g, 77%.

### Step 3. Preparation of Compound 88

Compound 88 is prepared from 87 (10 g, 19.8 mmol) using an identical procedure to that used for compound 85. Yield: 7.63 g, 65%.

### Step 4. Preparation of Compound 89

A solution of 88 (2 g, 3.38 mmol) and Z-glutamic acid (427 mg, 1.52 mmol) in CH₂Cl₂ (50 mL) is treated with HBTU (1.41 g, 3.7 mmol) and Hunig's base (1.77 mL, 10.1 mmol). After stirring (18h) the mixture is concentrated and subjected to chromatography to yield 89 (871 mg, 48%) as a colorless foam. Rf 0.5 (10% CH₃OH—CH₂Cl₂).

### Step 5. Preparation of Compound 90

A solution of 89 (870 mg, 0.72 mmol) and Pd/C (90 mg, 10%—wet support) in EtOAc (10 mL) is treated with TFA (84 μL, 1.1 mmol) and purged with H₂. After stirring vigorously (2h) the reaction is purged with N₂, filtered through Celite and concentrated. The crude material is used without further processing and yielded 90 (850 mg, quantitative) as a colorless foam. Rf 0.25 (10% CH₃OH—CH₂Cl₂).

### Step 6. Preparation of Compound 91

A solution of 90 (850 mg, 0.72 mmol) and Z-glutamic acid (91 mg, 0.32 mmol) in CH₂Cl₂ (10 mL) is treated with HBTU (300 mg, 0.79 mmol) and Hunig's base (502 μL, 2.9 mmol). After stirring (1.5h) the mixture is diluted with CH₂Cl₂ and washed with NaHCO₃(Sat. Aq.), dried (MgSO4), filtered and concentrated. The crude material is subjected to chromatography to yield 91 (590 mg, 76%) as a colorless foam. Rf 0.5 (10% CH₃OH—CH₂Cl₂).

### Step 7. Preparation of Compound 92

A solution of 91 (590 mg, 0.25 mmol) and Pd/C (100 mg, 10%—wet support) in CH₃OH (30 mL) is treated with TFA (29 μL, 0.37 mmol) and purged with H₂. After stirring (3h) the mixture is purged with N₂, then filtered through Celite and concentrated. The crude material is used without further processing and yielded 92 (600 mg, quantitative) as a colorless foam. Rf 0.1 (10% CH₃OH—CH₂Cl₂).

### Step 8. Preparation of Compound 101

Compound 101 is prepared from (R)-2-((2-oxo-2-phenyl-112-ethyl)amino)hexanedioic acid (2.51 g, 8.6 mmol) and 9 (11 g, 17.2 mmol) using an identical procedure to that used for compound 89. Yield: 4.2 g, 37%.

### Step 9. Preparation of Compound 102

Compound 102 is prepared from compound 101 (4.2 g, 3.2 mmol) using an identical procedure to that used for compound 90. Yield: 2.1 g, 47%.

### Step 10. Preparation of Compound 103

Compound 103 is prepared from (R)-2-((2-oxo-2-phenyl-112-ethyl)amino)hexanedioic acid (265 mg, 0.9 mmol) and compound 102 (2.1 g, 1.8 mmol) using an identical procedure to that used for compound 91. Yield: (560 mg, 24%).

### Step 11. Preparation of Compound 104

Compound 104 is prepared quantitatively from compound 103 (560 mg) using an identical procedure to that used for compound 92. The compound is used without purification.

### Step 12. Preparation of Conjugates 191, 194, and 197

Conjugates 191, 194, and 197 are prepared from compound 128 and 92, 96, and 100 using an identical procedure to that used for compound 1.

### Example 16a. Synthesis of Conjugates 191a

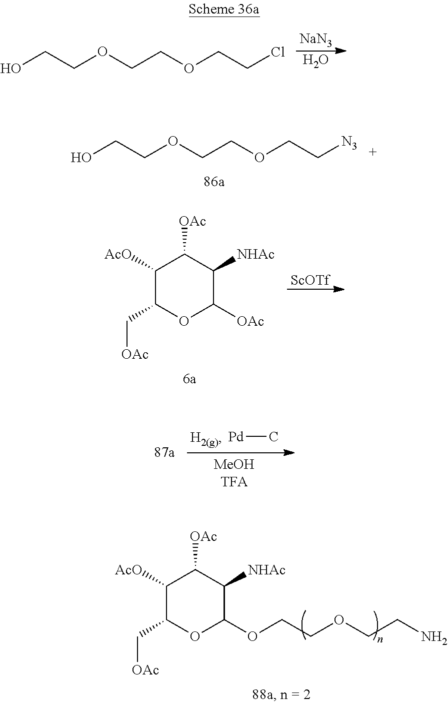

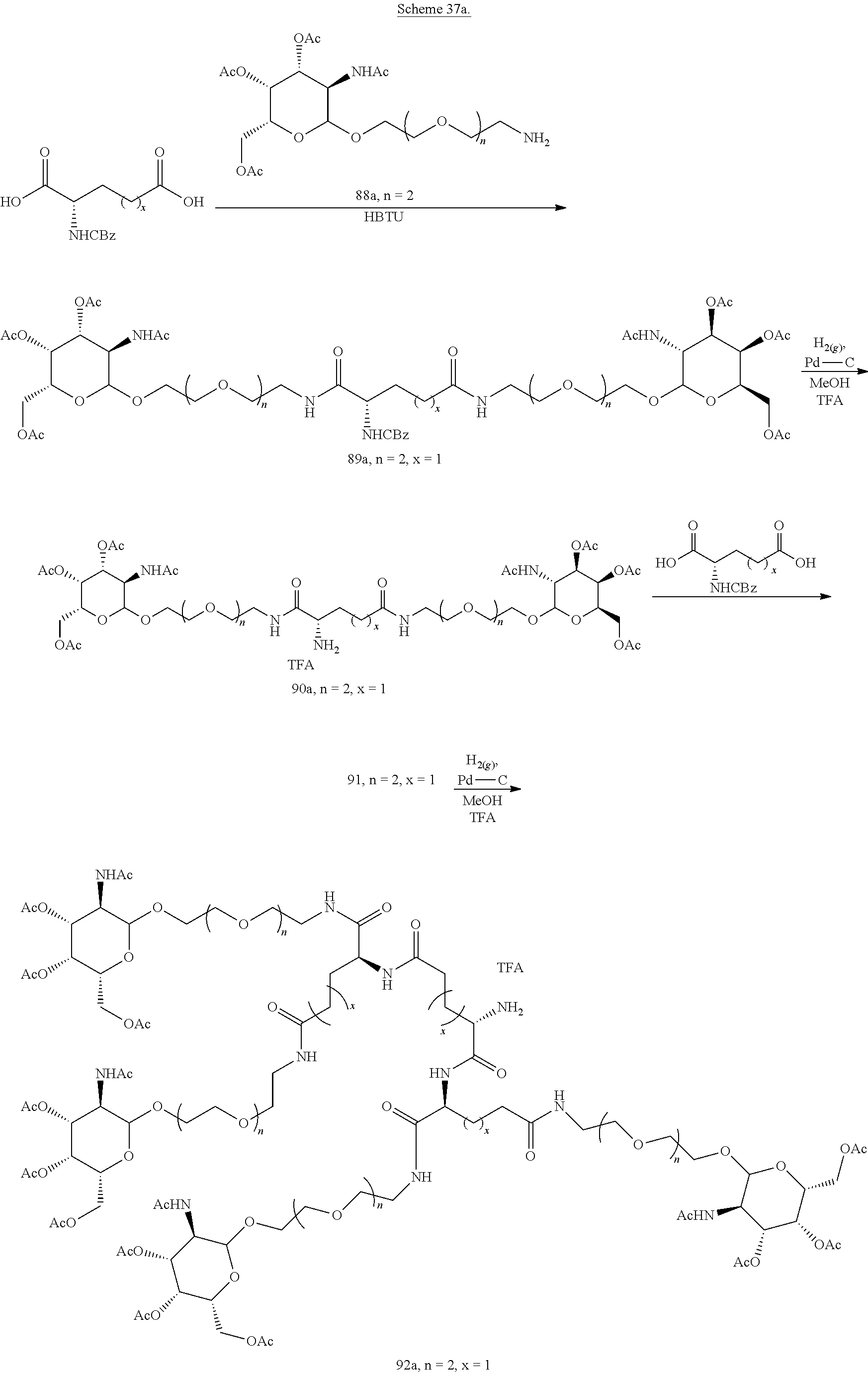

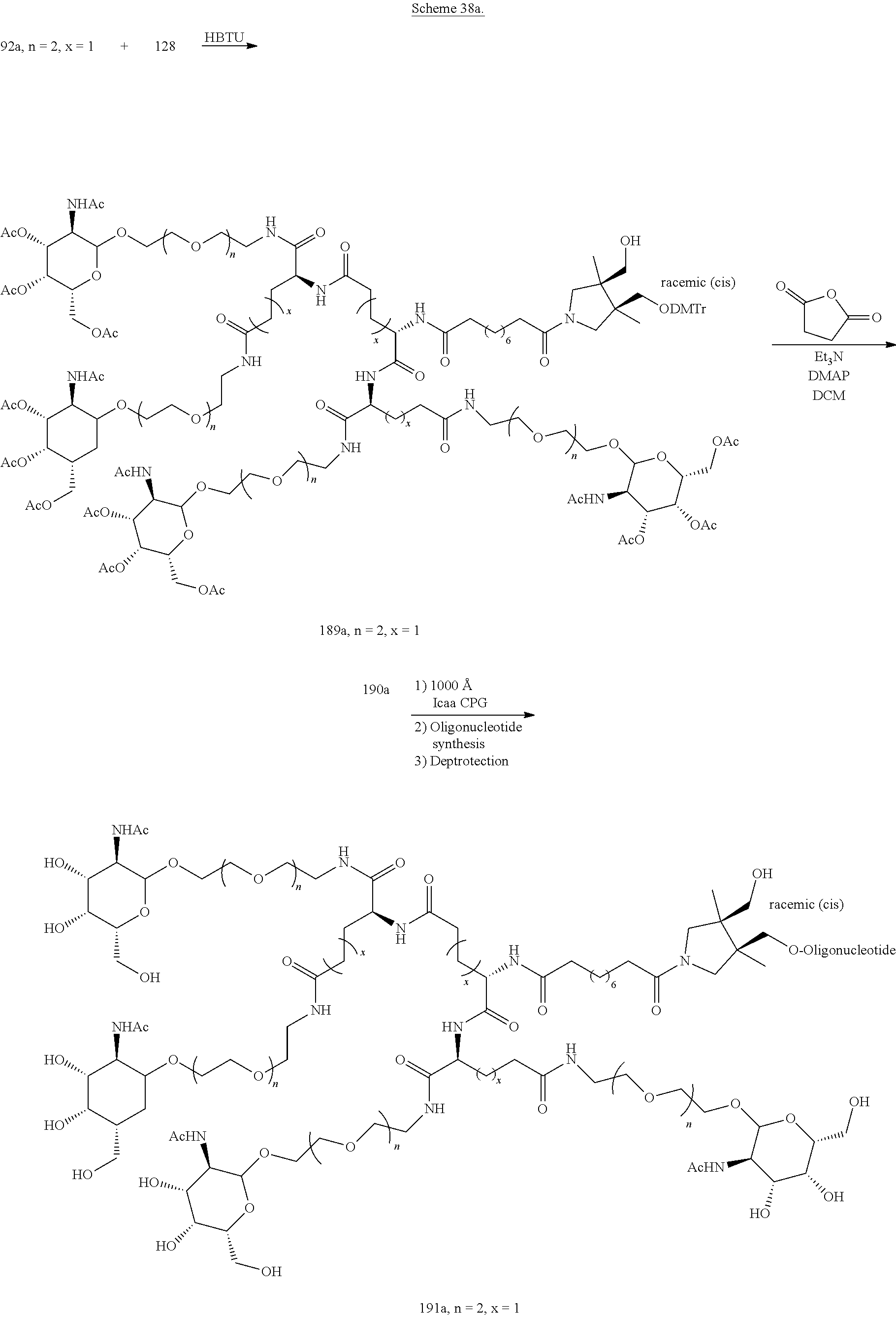

### Step 1. Preparation of 2-(2-(2-azidoethoxy)ethoxy)ethan-1-ol 86a

To a solution of 2-(2-(2-chloroethoxy)ethoxy)ethan-1-ol (13 g, 77 mmol) in water (200 mL) was added sodium azide (10 g, 154 mmol). The reaction was heated to 100° C. for 18 hours. The reaction was cooled to room temperature and poured into a 1 L separatory funnel and extracted with dichloromethane (3×200 mL). The combine dichloromethane extracts were dried on magnesium sulfate, filtered and concentrated to dryness to afford 2-(2-(2-azidoethoxy)ethoxy)ethan-1-ol as a colorless oil (11.7 g).

### Step 2. Preparation of Compound 87a

Compound 87a was prepared from 86a (4.95 g, 28.3 mmol) and 6a (10 g, 25.7 mmol) using an identical procedure to that used for compound 84. Yield: 10 g, 77%.

### Step 3. Preparation of Compound 88a

Compound 88a was prepared from 87a (10 g, 19.8 mmol) using an identical procedure to that used for compound 85. Yield: 7.63 g, 65%.

### Step 4. Preparation of Compound 89a

A solution of 88a (2 g, 3.38 mmol) and Z-L-glutamic acid (427 mg, 1.52 mmol) in CH₂Cl₂ (50 mL) was treated with HBTU (1.41 g, 3.7 mmol) and Hunig's base (1.77 mL, 10.1 mmol). After stirring (18h) the mixture was concentrated and subjected to chromatography to yield 89a (871 mg, 48%) as a colorless foam. Rf 0.5 (10% CH₃OH—CH₂Cl₂).

### Step 5. Preparation of Compound 90a

A solution of 89a (870 mg, 0.72 mmol) and Pd/C (90 mg, 10%—wet support) in EtOAc (10 mL) was treated with TFA (84 μL, 1.1 mmol) and purged with H₂. After stirring vigorously (2h) the reaction was purged with N₂, filtered through Celite and concentrated. The crude material was used without further processing and yielded 90a (850 mg, quantitative) as a colorless foam. Rf 0.25 (10% CH₃OH—CH₂Cl₂).

### Step 6. Preparation of Compound 91a

A solution of 90a (850 mg, 0.72 mmol) and Z-glutamic acid (91 mg, 0.32 mmol) in CH₂Cl₂ (10 mL) was treated with HBTU (300 mg, 0.79 mmol) and Hunig's base (502 μL, 2.9 mmol). After stirring (1.5h) the mixture diluted with CH₂Cl₂ and washed with NaHCO₃ (Sat. Aq.), dried (MgSO₄), filtered and concentrated. The crude material was subjected to chromatography to yield 91a (590 mg, 76%) as a colorless foam. Rf 0.5 (10% CH₃OH—CH₂Cl₂).

### Step 7. Preparation of Compound 92a

A solution of 91a (590 mg, 0.25 mmol) and Pd/C (100 mg, 10%—wet support) in CH₃OH (30 mL) was treated with TFA (29 μL, 0.37 mmol) and purged with H₂. After stirring (3h) the mixture was purged with N₂, then filtered through Celite and concentrated. The crude material was used without further processing and yielded 92a (600 mg, quantitative) as a colorless foam. Rf 0.1 (10% CH₃OH—CH₂Cl₂).

### Step 8. Preparation of Conjugate 191a

Conjugate 191a was prepared from compound 128 and compound 92a using an identical procedure to that used for compound 1.

### Example 16b. Synthesis of Conjugates 191b

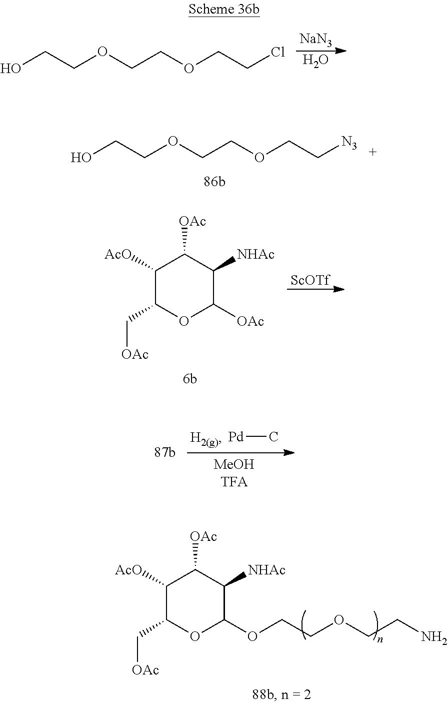

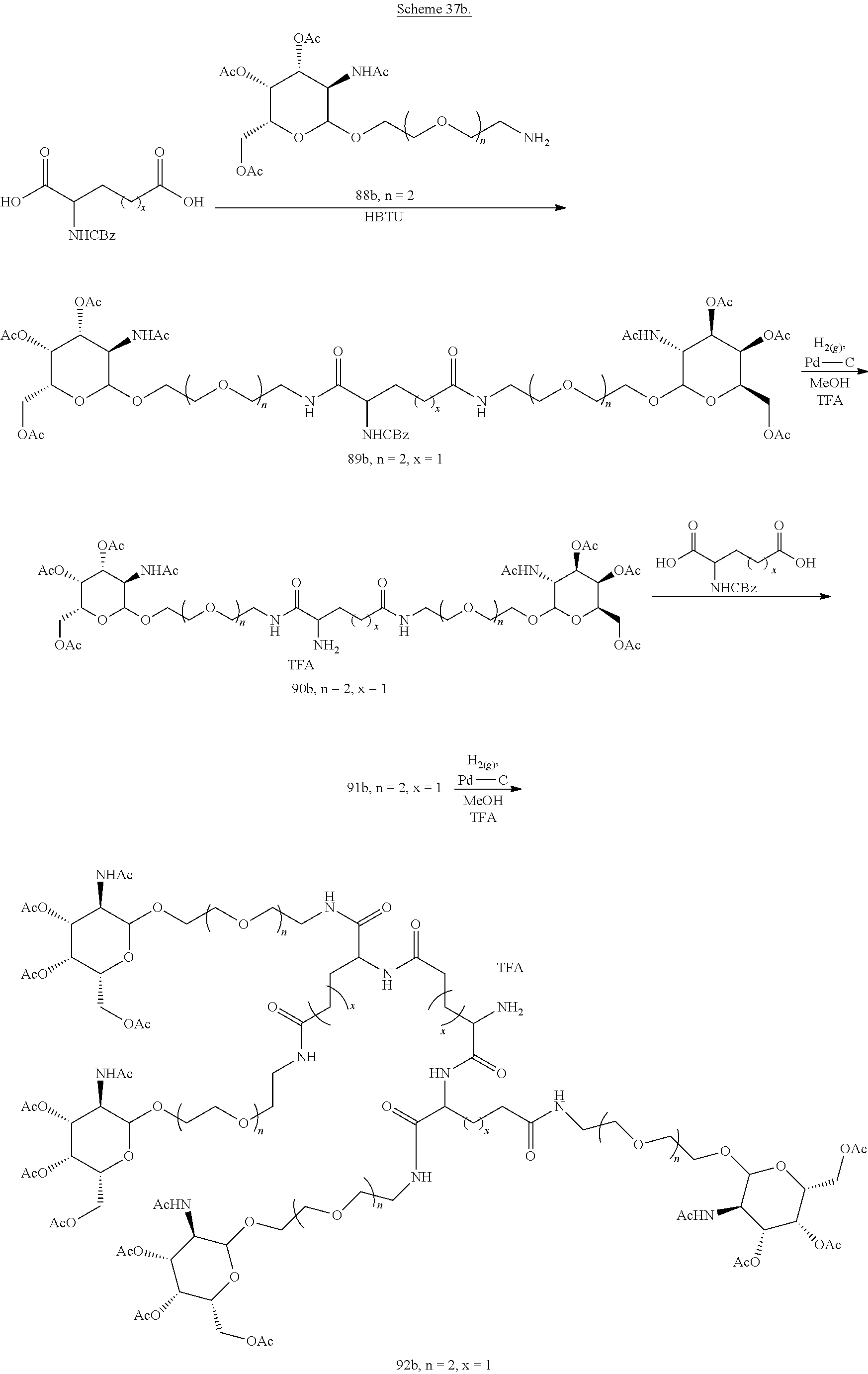

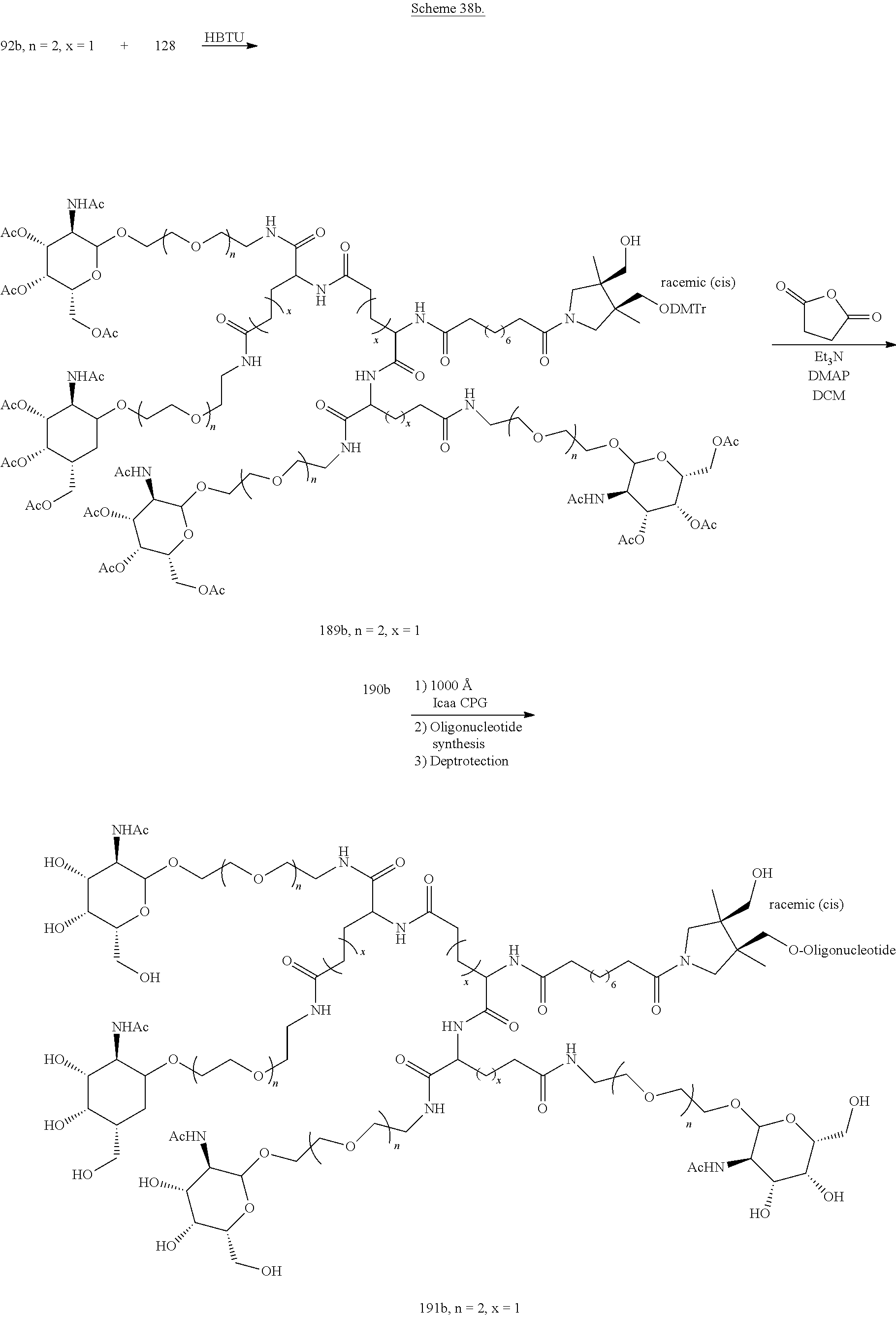

### Step 1. Preparation of 2-(2-(2-azidoethoxy)ethoxy)ethan-1-ol 86b

To a solution of 2-(2-(2-chloroethoxy)ethoxy)ethan-1-ol (13 g, 77 mmol) in water (200 mL) is added sodium azide (10 g 154 mmol). The reaction was heated to 100° C. for 18 hours. The reaction was cooled to room temperature and poured into a 1 L separatory funnel and extracted with dichloromethane (3×200 mL). The combine dichloromethane extracts were dried on magnesium sulfate, filtered and concentrated to dryness to afford 2-(2-(2-azidoethoxy)ethoxy)ethan-1-ol as a colorless oil (11.7 g).

### Step 2. Preparation of Compound 87b

Compound 87a is prepared from 86b (4.95 g, 28.3 mmol) and 6b (10 g, 25.7 mmol) using an identical procedure to that used for compound 84. Yield: 10 g, 77%.

### Step 3. Preparation of Compound 88b

Compound 88a is prepared from 87b (10 g, 19.8 mmol) using an identical procedure to that used for compound 85. Yield: 7.63 g 65%.

### Step 4. Preparation of Compound 89b

A solution of 88b (2 g, 3.38 mmol) and racemic Z-glutamic acid (427 mg 1.52 mmol) in CH₂Cl₂ (50 mL) is treated with HBTU (1.41 g 3.7 mmol) and Hunig's base (1.77 mL, 10.1 mmol). After stirring (18 h) the mixture was concentrated and subjected to chromatography to yield 89b (871 mg, 48%) as a colorless foam. Rf 0.5 (10 CH₃OH—CH₂Cl₂).

### Step 5. Preparation of Compound 90b

A solution of 89b (870 mg, 0.72 mmol) and Pd/C (90 mg, 10%—wet support) in EtOAc (10 mL) is treated with TFA (84 μL, 1.1 mmol) and purged with H₂. After stirring vigorously (2h) the reaction is purged with N₂, filtered through Celite and concentrated. The crude material is used without further processing and yielded 90b (850 mg, quantitative) as a colorless foam. Rf 0.25 (10% CH₃OH—CH₂Cl₂).

### Step 6. Preparation of Compound 91b

A solution of 90b (850 mg, 0.72 mmol) and Z-glutamic acid (91 mg, 0.32 mmol) in CH₂Cl₂ (10 mL) is treated with HBTU (300 mg, 0.79 mmol) and Hunig's base (502 μL, 2.9 mmol). After stirring (1.5h) the mixture is diluted with CH₂Cl₂ and washed with NaHCO₃ (Sat. Aq.), dried (MgSO₄), filtered and concentrated. The crude material is subjected to chromatography to yield 91b (590 mg, 76%) as a colorless foam. Rf 0.5 (10% CH₃OH—CH₂Cl₂).

### Step 7. Preparation of Compound 92b

A solution of 91b (590 mg, 0.25 mmol) and Pd/C (100 mg, 10%—wet support) in CH₃OH (30 mL) is treated with TFA (29 μL, 0.37 mmol) and purged with H₂. After stirring (3h) the mixture is purged with N₂, then filtered through Celite and concentrated. The crude material is used without further processing and yielded 92b (600 mg, quantitative) as a colorless foam. Rf 0.1 (10% CH₃OH—CH₂Cl₂).

### Step 8. Preparation of Conjugate 191b

Conjugate 191b is prepared from compound 128 and compound 92b using an identical procedure to that used for compound 1.

### Example 16c. Synthesis of Conjugates 191c

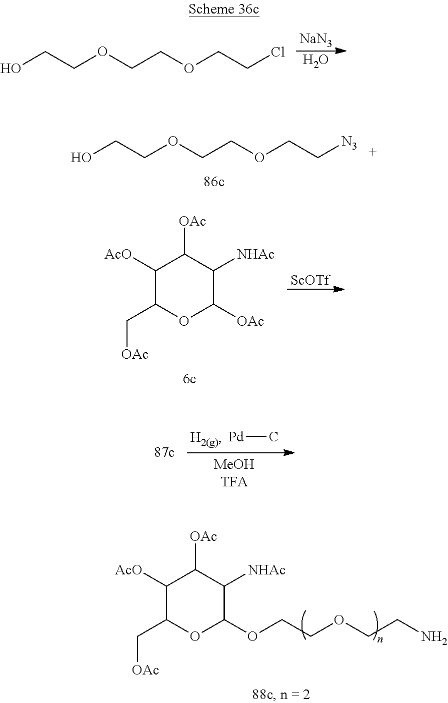

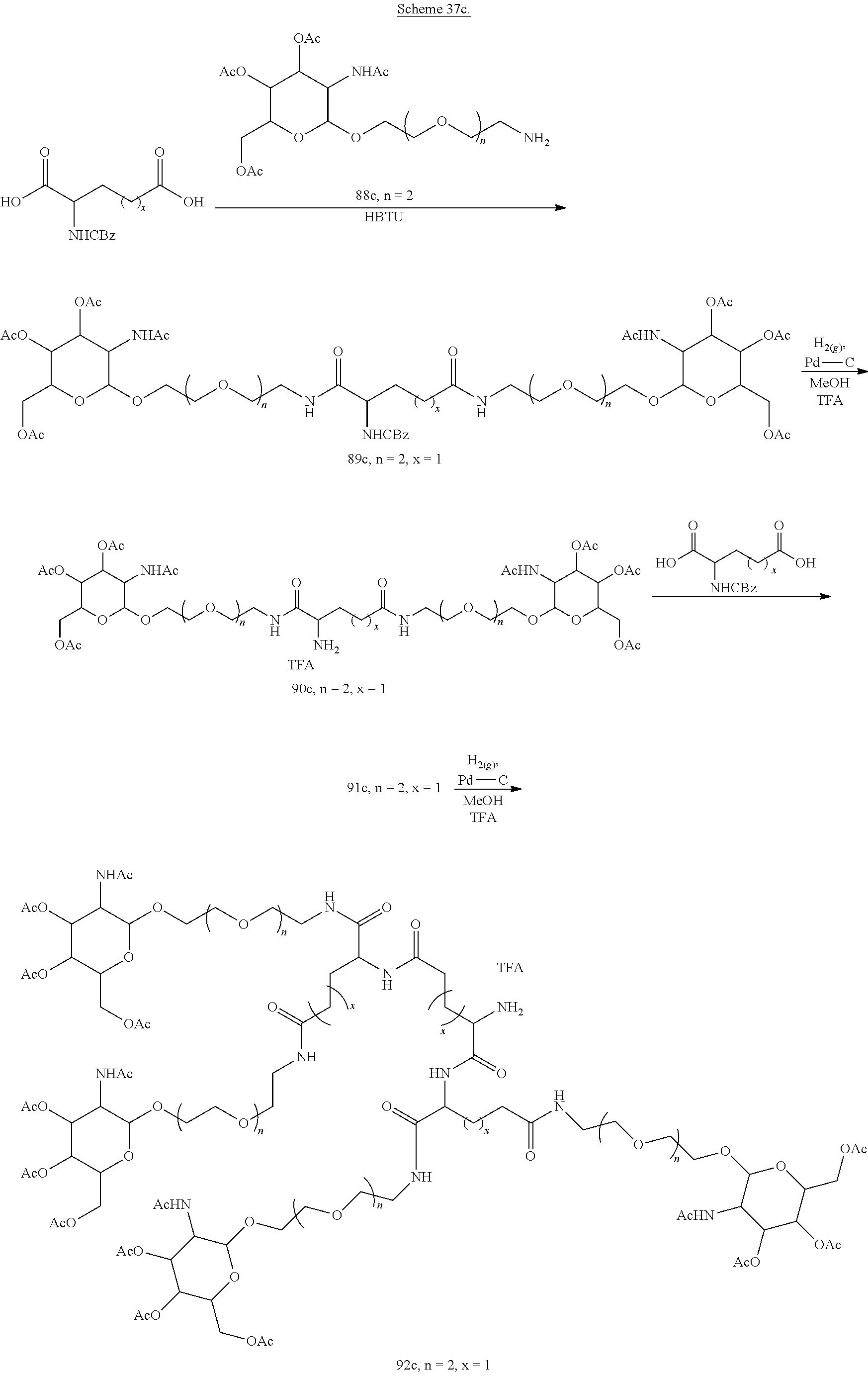

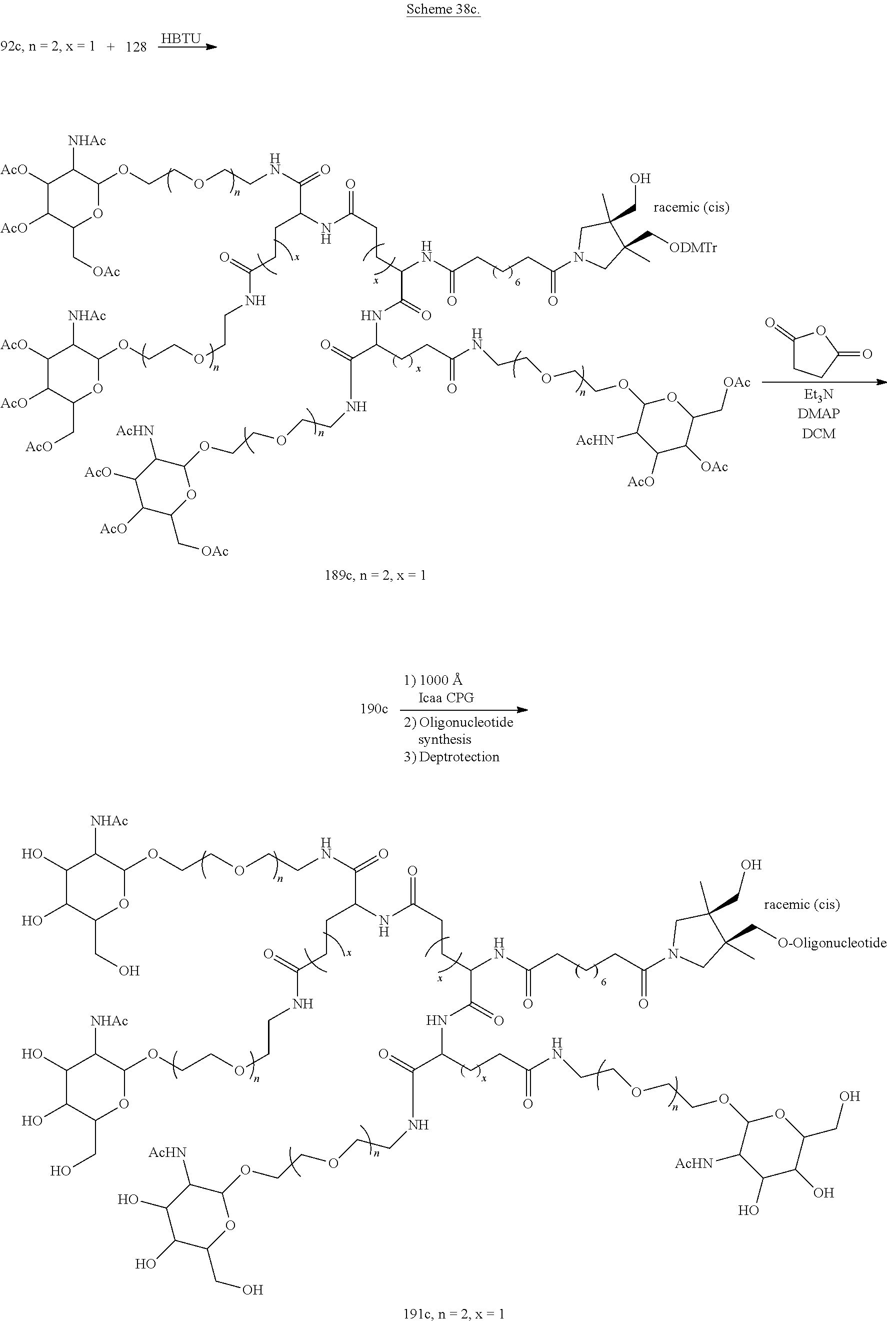

### Step 1. Preparation of 2-(2-(2-azidoethoxy)ethoxy)ethan-1-ol 86c

To a solution of 2-(2-(2-chloroethoxy)ethoxy)ethan-1-ol (13 g, 77 mmol) in water (200 mL) is added sodium azide (10 g, 154 mmol). The reaction was heated to 100° C. for 18 hours. The reaction was cooled to room temperature and poured into a 1 L separatory funnel and extracted with dichloromethane (3×200 mL). The combine dichloromethane extracts were dried on magnesium sulfate, filtered and concentrated to dryness to afford 2-(2-(2-azidoethoxy)ethoxy)ethan-1-ol as a colorless oil (11.7 g).

### Step 2. Preparation of Compound 87c

Compound 87c is prepared from 86c (4.95 g, 28.3 mmol) and 6c (10 g, 25.7 mmol) using an identical procedure to that used for compound 84. Yield: 10 g, 77%.

### Step 3. Preparation of Compound 88c

Compound 88c is prepared from 87c (10 g, 19.8 mmol) using an identical procedure to that used for compound 85. Yield: 7.63 g, 65%.

### Step 4. Preparation of Compound 89c

A solution of 88c (2 g, 3.38 mmol) and racemic Z-glutamic acid (427 mg, 1.52 mmol) in CH₂Cl₂ (50 mL) is treated with HBTU (1.41 g, 3.7 mmol) and Hunig's base (1.77 mL, 10.1 mmol). After stirring (18h) the mixture was concentrated and subjected to chromatography to yield 89c (871 mg, 48%) as a colorless foam. Rf 0.5 (10% CH₃OH—CH₂Cl₂).

### Step 5. Preparation of Compound 90c

A solution of 89c (870 mg, 0.72 mmol) and Pd/C (90 mg, 10%—wet support) in EtOAc (10 mL) is treated with TFA (84 μL, 1.1 mmol) and purged with H₂. After stirring vigorously (2h) the reaction is purged with N₂, filtered through Celite and concentrated. The crude material is used without further processing and yielded 90c (850 mg, quantitative) as a colorless foam. Rf 0.25 (10% CH₃OH—CH₂Cl₂).

### Step 6. Preparation of Compound 91c

A solution of 90c (850 mg, 0.72 mmol) and Z-glutamic acid (91 mg, 0.32 mmol) in CH₂Cl₂ (10 mL) is treated with HBTU (300 mg, 0.79 mmol) and Hunig's base (502 μL, 2.9 mmol). After stirring (1.5h) the mixture is diluted with CH₂Cl₂ and washed with NaHCO₃ (Sat. Aq.), dried (MgSO₄), filtered and concentrated. The crude material is subjected to chromatography to yield 91c (590 mg, 76%) as a colorless foam. Rf 0.5 (10% CH₃OH—CH₂Cl₂).

### Step 7. Preparation of Compound 92c

A solution of 91c (590 mg, 0.25 mmol) and Pd/C (100 mg, 10%—wet support) in CH₃OH (30 mL) is treated with TFA (29 μL, 0.37 mmol) and purged with H₂. After stirring (3h) the mixture is purged with N₂, then filtered through Celite and concentrated. The crude material is used without further processing and yielded 92c (600 mg, quantitative) as a colorless foam. Rf 0.1 (10% CH₃OH—CH₂Cl₂).

### Step 8. Preparation of Conjugate 191c

Conjugate 191c is prepared from compound 128 and compound 92c using an identical procedure to that used for compound 1.

### Example 17. Synthesis of Conjugates 203 and 206

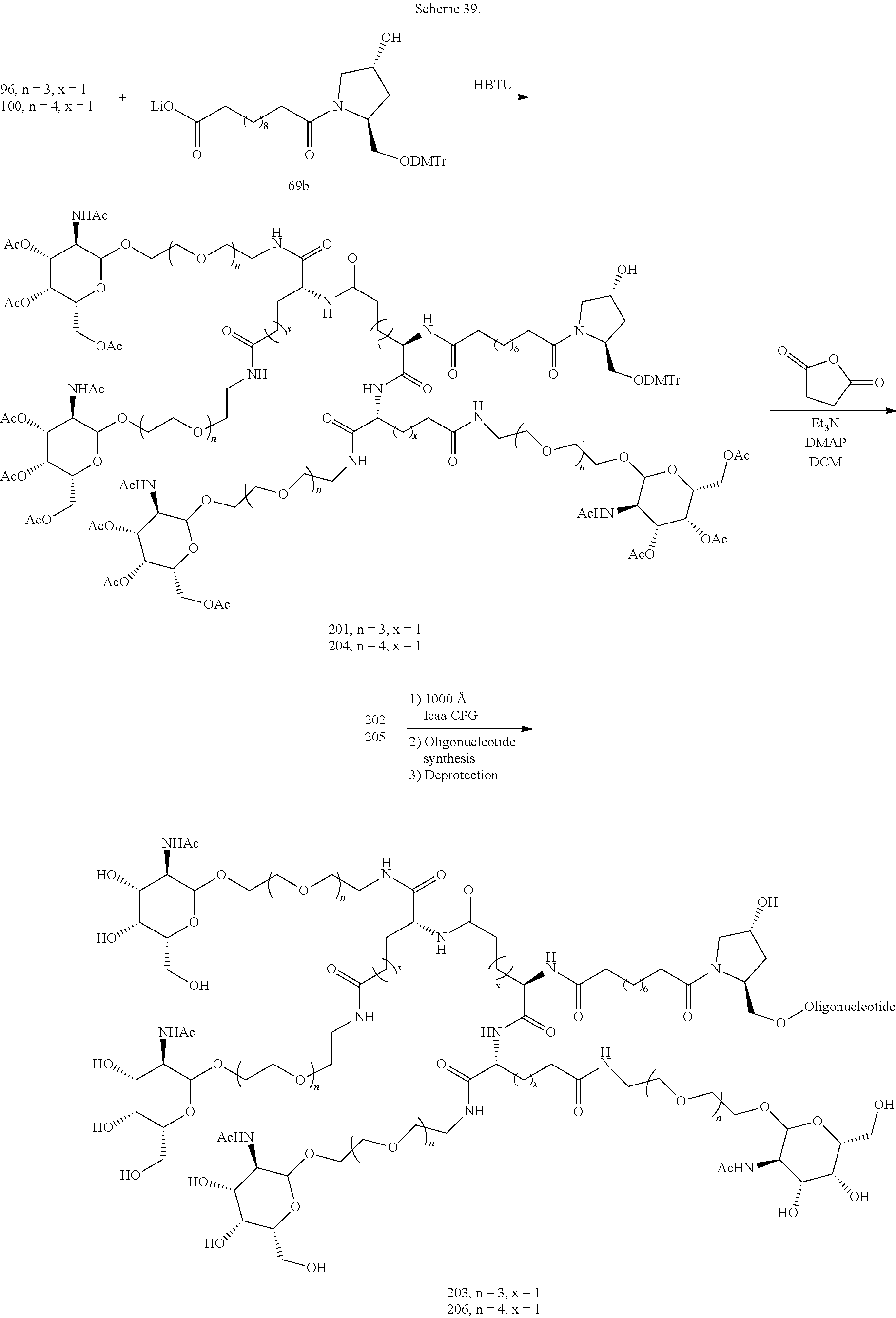

### Step 1. Preparation of Compound 69b

Compound 69b was prepared from (2S,4R)-4-Hydroxypyrrolidine-2-carboxylic acid using an identical procedure to that used for compound 69.

### Step 2. Preparation of Conjugates 203 and 206

Conjugates 203 and 206 were prepared from compound 96 and 100 using an identical procedure to that used for compound 1.

### Example 18. Synthesis of Conjugate 209

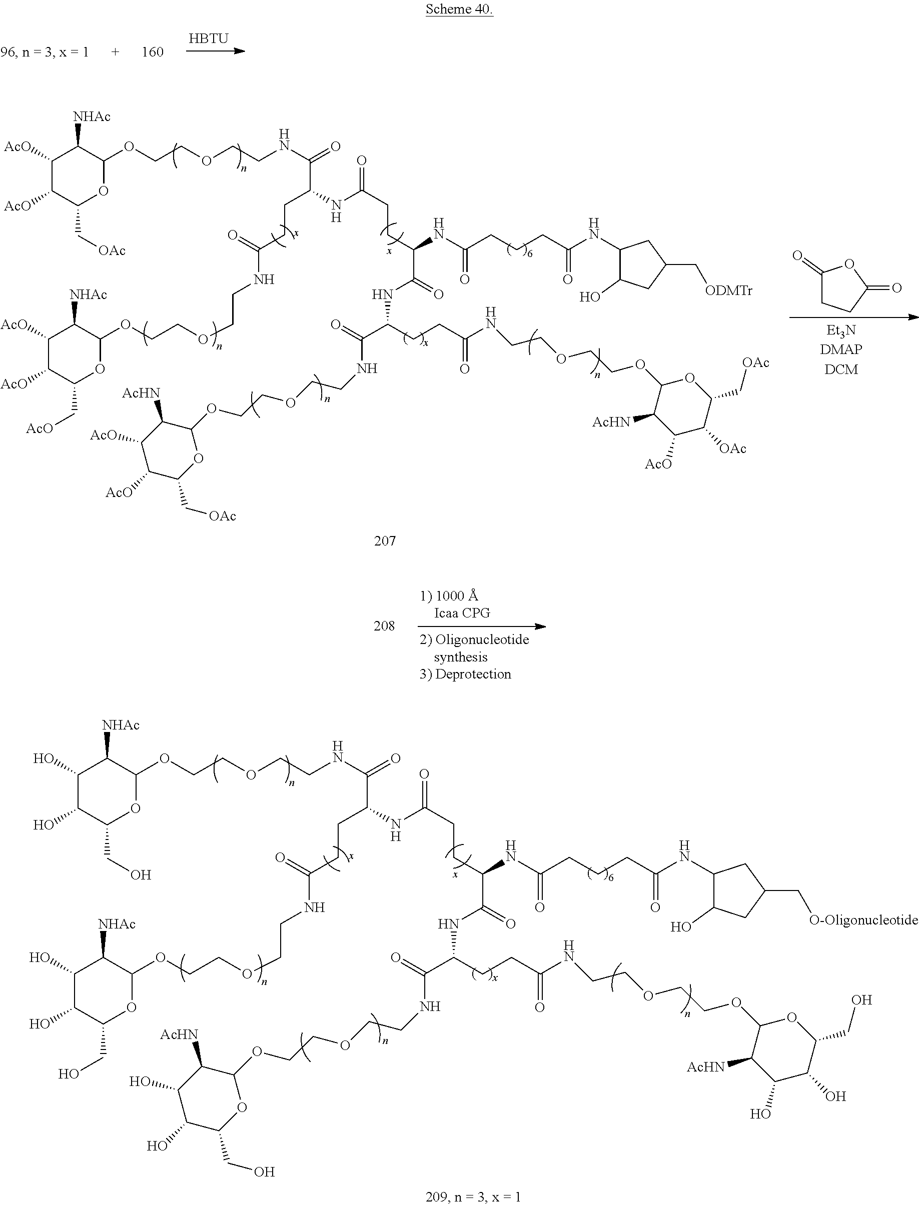

### Step 1. Preparation of Conjugate 209

Conjugate 209 was prepared from compound 96 and 160 using an identical procedure to that used for compound 1.

### Example 18a. Synthesis of Conjugate 209a

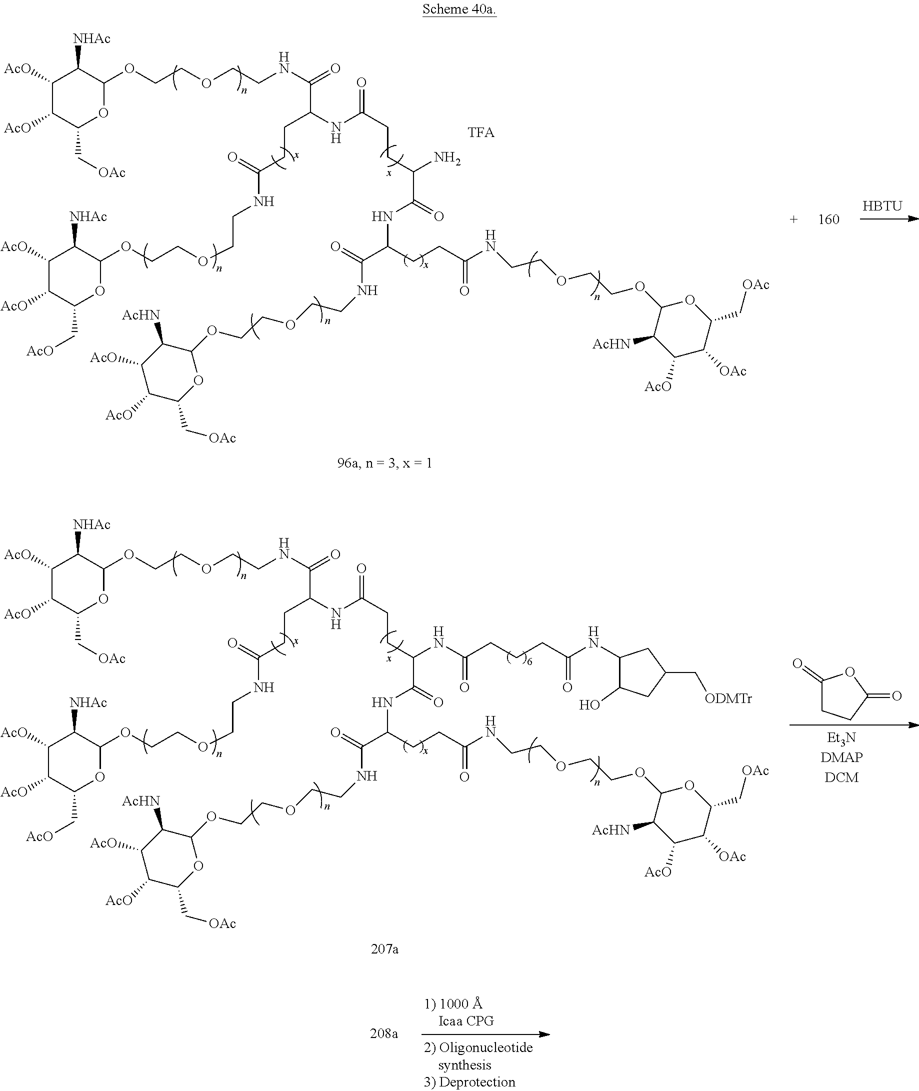

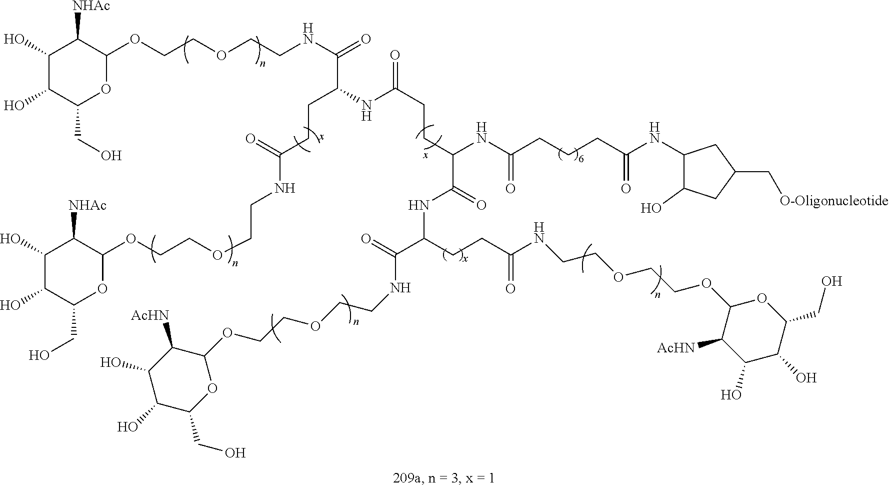

### Step 1. Preparation of Conjugate 209a

Conjugate 209a is prepared from compound 96a and 160 using an identical procedure to that used for compound 1.

### Example 19. Synthesis of Conjugates 212 and 215

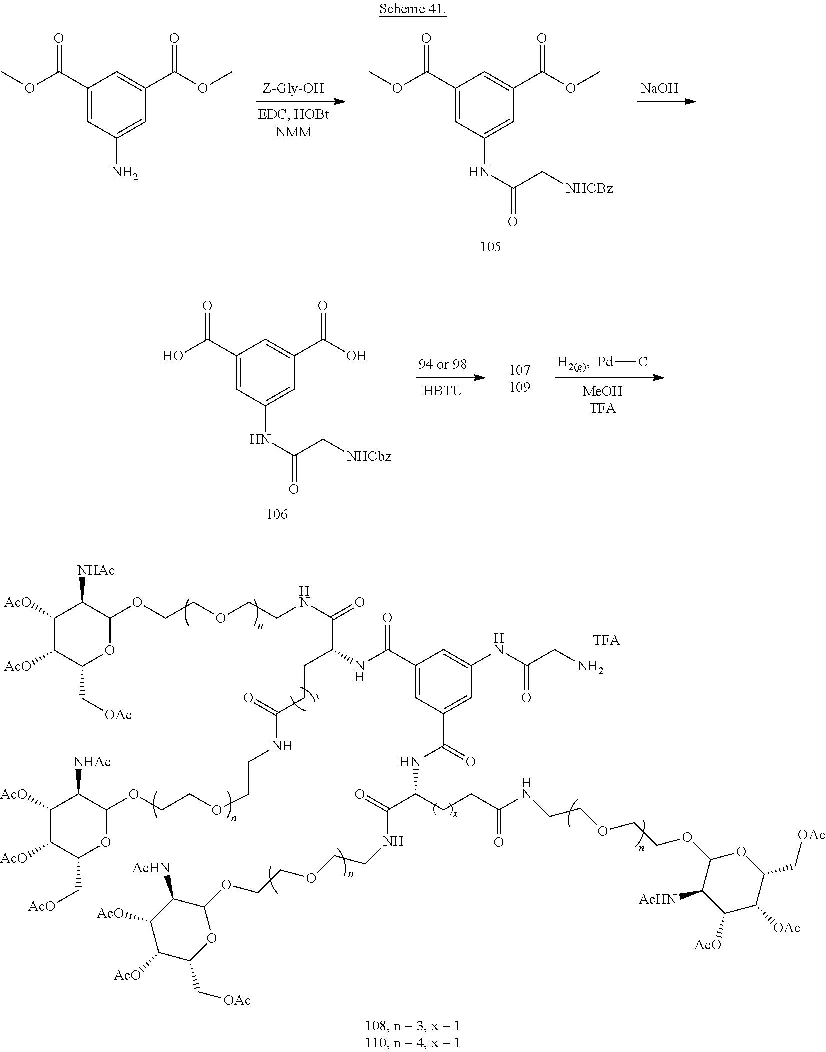

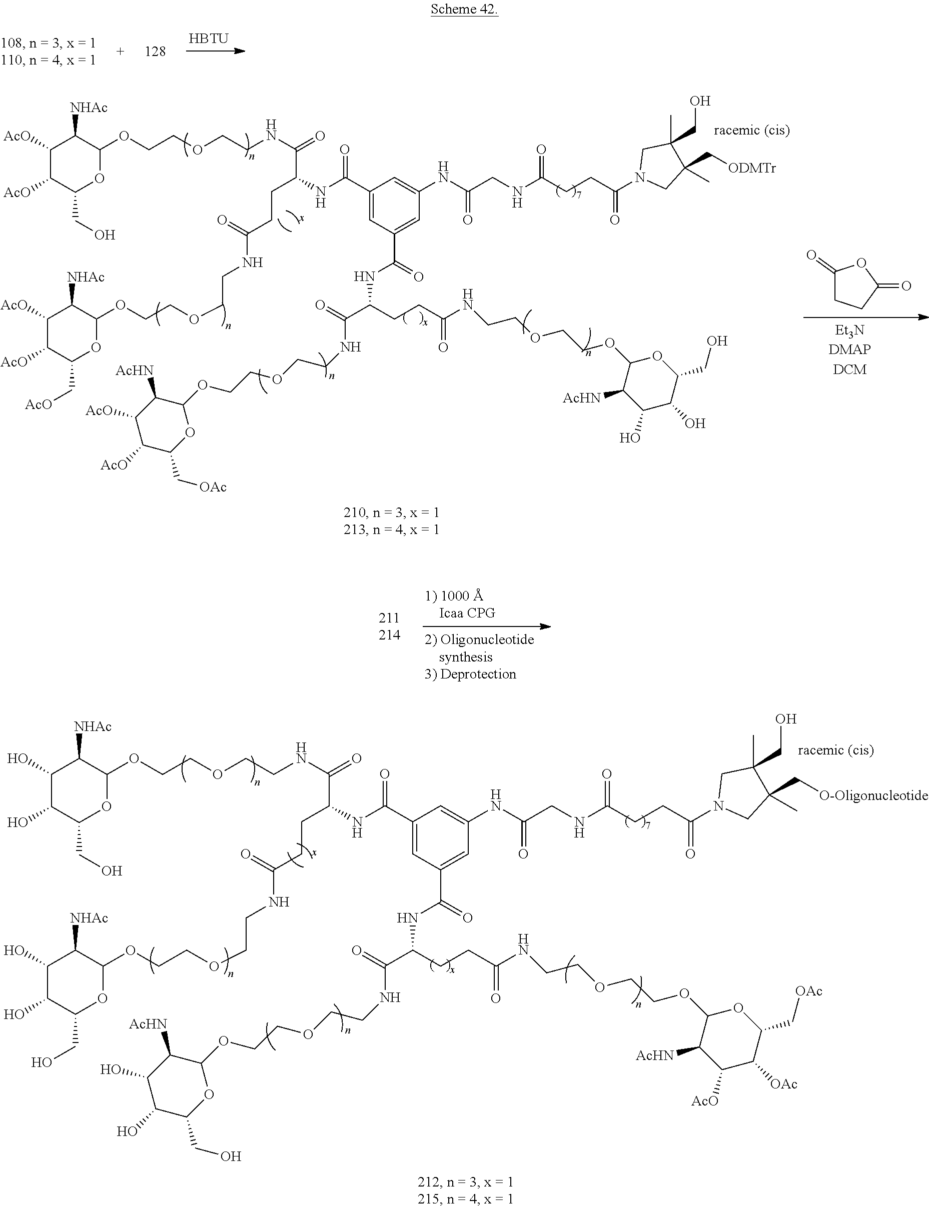

### Step 1. Preparation of Dimethyl 5-(2-((2-oxo-2-phenyl-1λ²-ethyl)amino)acetamido)-isophthalate 105

A solution of dimethyl 5-aminoisophthalate (5 g, 24 mmol), Z-Gly-OH (5 g, 24 mmol), EDC (5 g, 26.3 mmol), HOBt (3.6 g, 26.3 mmol), NMM (2.9 mL, 26.3 mmol) in DMF (50 mL) was stirred overnight at room temperature. Upon completion, the reaction mixture was diluted with ethyl acetate (250 mL) and washed with each 1M HCl (2×100 mL), saturated sodium bicarbonate (1×100 mL) and brine (2×100 mL). Dry on magnesium sulfate, filter and concentrate to dryness to afford Dimethyl 5-(2-((2-oxo-2-phenyl-1λ²-ethyl)amino)acetamido)isophthalate as a colorless solid (7.2 g, 79%).

### Step 2. Preparation of 5-(2-((2-oxo-2-phenyl-1λ²-ethyl)amino)acetamido)isophthalic acid 106

To a solution of methyl 5-(2-((2-oxo-2-phenyl-1λ²-ethyl)amino)acetamido)isophthalate (7.2 g) in methanol (25 mL) and THF (25 mL) was added 1M NaOH (25 mL). The solution was stirred at room temperature for 2 hours then concentrated to remove THF and MeOH. The aqueous solution remaining was diluted with water (75 mL), cooled on an ice water bath and acidified to pH=1 with 6M HCl. The solid was filtered and washed with water (3×100 mL).

The solid was freeze dried to afford 5-(2-((2-oxo-2-phenyl-1λ²-ethyl)amino)acetamido)-isophthalic acid (6.9 g, quantitative).

### Step 3. Preparation of Compound 107

Compound 107 was prepared from 5-(2-((2-oxo-2-phenyl-1λ²-ethyl)amino)acetamido)isophthalic acid 106 (200 mg, 0.54 mmol) and 94 (1.7 g, 1.3 mmol) using an identical procedure to that used for compound 95. Yield: 600 mg.

### Step 4. Preparation of Compound 108

Compound 108 was prepared from compound 107 (600 mg) using an identical procedure to that used for compound 96. Yield: 650 mg, quantitative.

### Step 5. Preparation of Compound 109

Compound 109 was prepared from 5-(2-((2-oxo-2-phenyl-1λ²-ethyl)amino)acetamido)isophthalic acid 106 (180 mg, 0.48 mmol) and 98 (1.5 g, 1.1 mmol) using an identical procedure to that used for compound 99. Yield: 900 mg.

### Step 6. Preparation of Compound 110

Compound 110 was prepared from compound 109 (900 mg) using an identical procedure to that used for compound 100. Yield: 920 mg, quantitative.

### Step 7. Preparation of Conjugates 212 and 215

Conjugates 212 and 215 were prepared from compound 128 and 108 or 110 using an identical procedure to that used for compound 1.

### Example 19a. Synthesis of Conjugates 212a and 215a

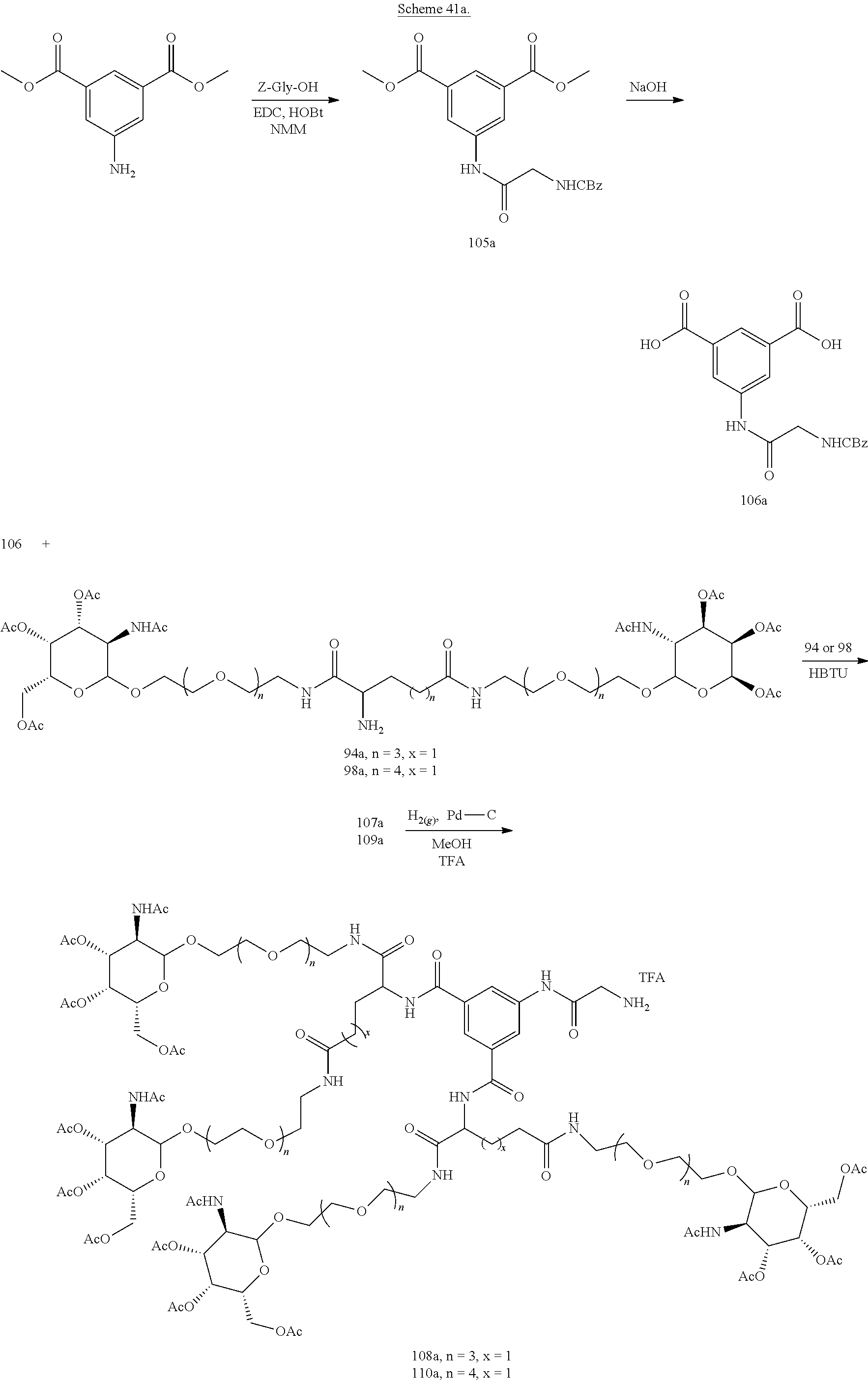

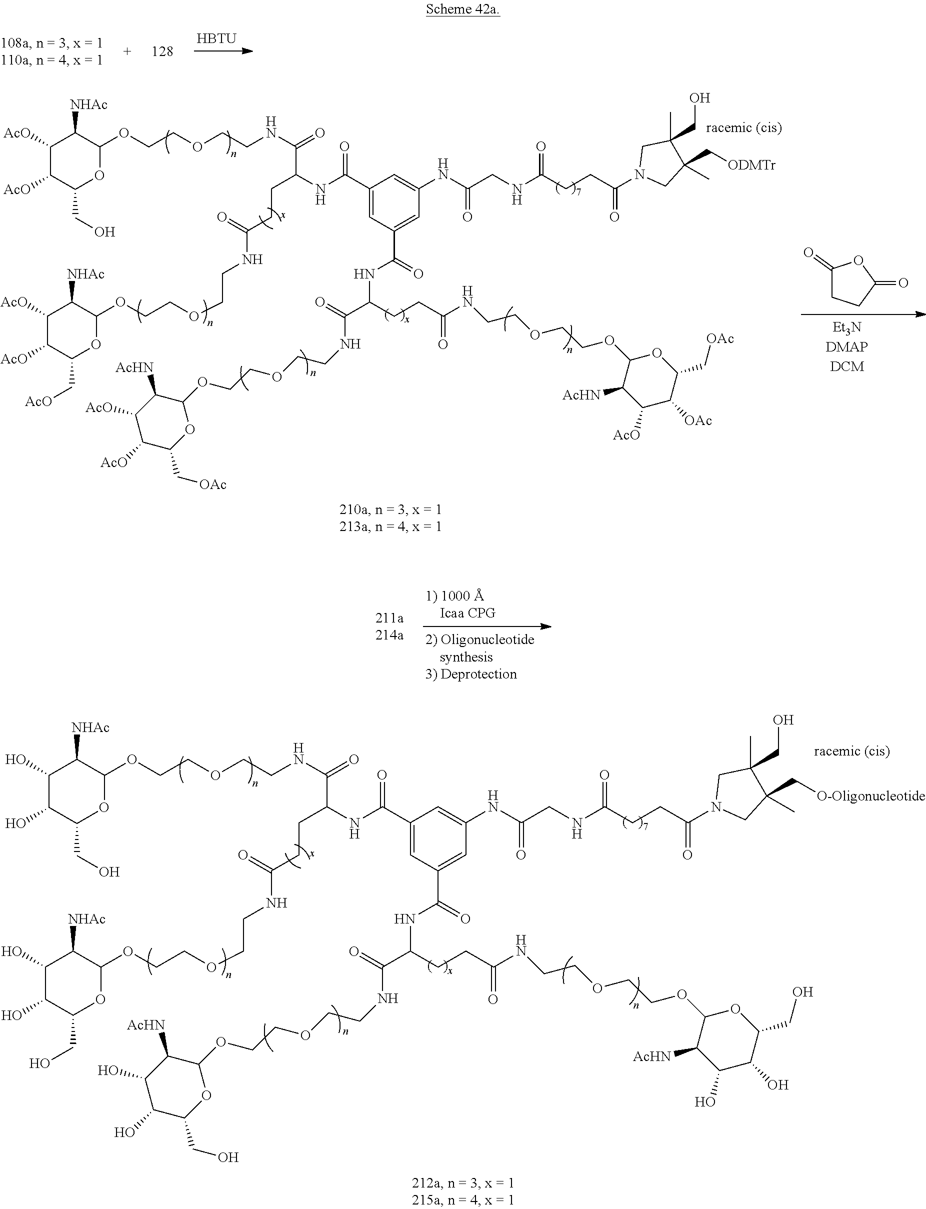

### Step 1. Preparation of Dimethyl 5-(2-((2-oxo-2-phenyl-1λ²-ethyl)amino)acetamido)-isophthalate 105a

A solution of dimethyl 5-aminoisophthalate (5 g, 24 mmol), Z-Gly-OH (5 g, 24 mmol), EDC (5 g, 26.3 mmol), HOBt (3.6 g, 26.3 mmol), NMM (2.9 mL, 26.3 mmol) in DMF (50 mL) is stirred overnight at room temperature. Upon completion, the reaction mixture is diluted with ethyl acetate (250 mL) and washed with each 1M HCl (2×100 mL), saturated sodium bicarbonate (1×100 mL) and brine (2×100 mL). Dry on magnesium sulfate, filter and concentrate to dryness to afford Dimethyl 5-(2-((2-oxo-2-phenyl-1λ²-ethyl)amino)acetamido)isophthalate as a colorless solid (7.2 g, 79%).

### Step 2. Preparation of 5-(2-((2-oxo-2-phenyl-1λ²-ethyl)amino)acetamido)isophthalic acid 106a

To a solution of methyl 5-(2-((2-oxo-2-phenyl-1λ²-ethyl)amino)acetamido)isophthalate (7.2 g) in methanol (25 mL) and THF (25 mL) is added 1M NaOH (25 mL). The solution is stirred at room temperature for 2 hours then concentrated to remove THF and MeOH. The aqueous solution remaining is diluted with water (75 mL), cooled on an ice water bath and acidified to pH=1 with 6M HCl. The solid is filtered and washed with water (3×100 mL). The solid is freeze dried to afford 5-(2-((2-oxo-2-phenyl-1λ²-ethyl)amino)acetamido)-isophthalic acid (6.9 g, quantitative).

### Step 3. Preparation of Compound 107a

Compound 107a is prepared from 5-(2-((2-oxo-2-phenyl-1λ²-ethyl)amino)acetamido)isophthalic acid 106a (200 mg, 0.54 mmol) and 94a (1.7 g, 1.3 mmol) using an identical procedure to that used for compound 95. Yield: 600 mg.

### Step 4. Preparation of Compound 108a

Compound 108a is prepared from compound 107a (600 mg) using an identical procedure to that used for compound 96a. Yield: 650 mg, quantitative.

### Step 5. Preparation of Compound 109a

Compound 109a is prepared from 5-(2-((2-oxo-2-phenyl-1λ²-ethyl)amino)acetamido)isophthalic acid 106a (180 mg, 0.48 mmol) and 9a8 (1.5 g, 1.1 mmol) using an identical procedure to that used for compound 99. Yield: 900 mg.

### Step 6. Preparation of Compound 110a

Compound 110a is prepared from compound 109 (900 mg) using an identical procedure to that used for compound 100. Yield: 920 mg, quantitative.

### Step 7. Preparation of Conjugates 212a and 215a

Conjugates 212a and 21a5 are prepared from compound 128 and 108a or 110a using an identical procedure to that used for compound 1.

### Example 20. Synthesis of Conjugates 218 and 221

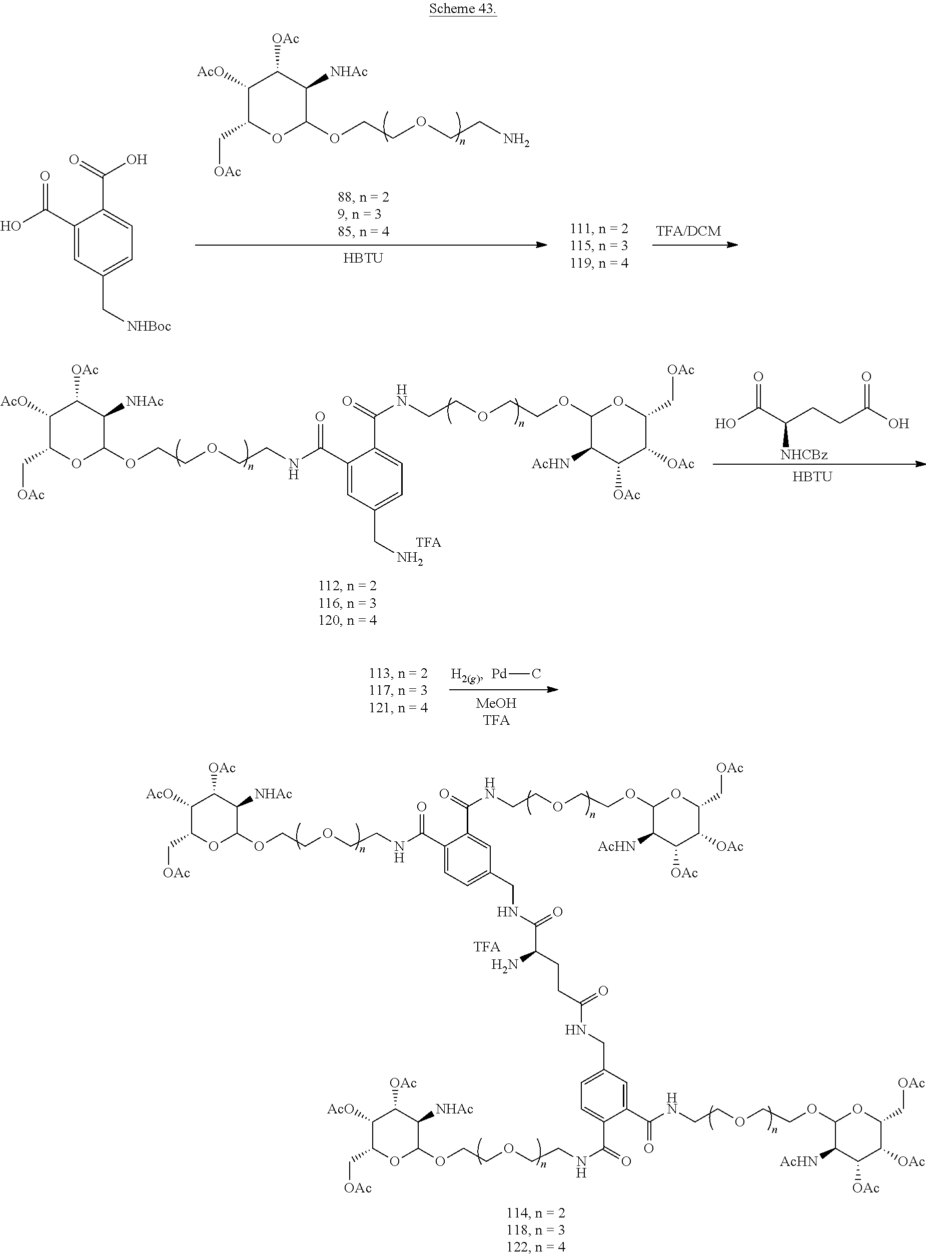

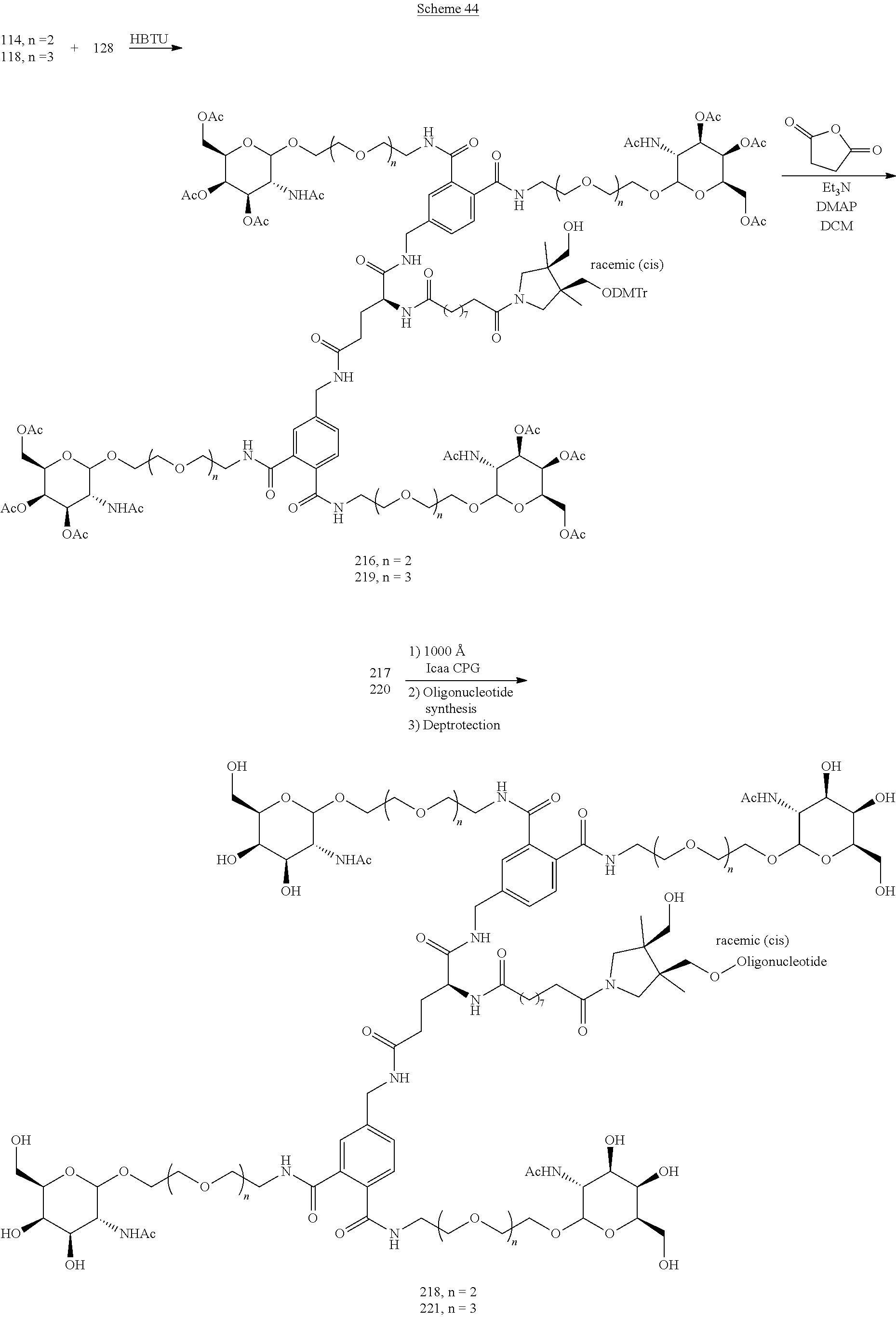

### Step 1. Preparation of Compound 111

Compound 111 was prepared from 4-(((tert-butoxycarbonyl)amino)methyl)phthalic acid (1.13 g, 3.84 mmol) and 88 (5 g, 8.44 mmol) using an identical procedure to that used for compound 89. Yield: 2.21 g, 49%.

### Step 2. Preparation of Compound 112

A solution of 111 (2.21 g, 1.87 mmol) in CH₂Cl₂ (40 mL) was slowly treated with TFA (5 mL). After stirring (2h) the mixture was concentrated and subjected to chromatography to yield 112 (1.08 g, 47%) as a colorless foam. Rf 0.1 (10% CH₃OH—CH₂Cl₂).

### Step 3. Preparation of Compound 113

Compound 113 was prepared from compound 112 (1.08 g, 0.88 mmol) and (2-oxo-2-phenyl-1λ²-ethyl)-D-glutamic acid (112 mg, 0.39 mmol) using an identical procedure to that used for compound 91. Yield: 600 mg, 62%.

### Step 4. Preparation of Compound 114

Compound 114 was prepared from compound 113 using an identical procedure to that used for compound 92.

### Step 5. Preparation of Compound 115

Compound 115 was prepared from 4-(((tert-butoxycarbonyl)amino)methyl)phthalic acid (3.94 g, 13.3 mmol) and 9 (18.2 g, 29.4 mmol) using an identical procedure to that used for compound 93. Yield: 9.02 g, 53%.

### Step 6. Preparation of Compound 116

Compound 116 was prepared from compound 115 (8 g, 6.3 mmol) using an identical procedure to that used for compound 112. Yield: 3.23 g, 39%.

### Step 7. Preparation of Compound 117

Compound 117 was prepared from compound 116 (3.23 g, 2.45 mmol) and (2-oxo-2-phenyl-1λ²-ethyl)-D-glutamic acid (192 mg, 1.1 mmol) using an identical procedure to that used for compound 95. Yield: 2.22 g, 34%.

### Step 8. Preparation of Compound 118

Compound 118 was prepared from compound 117 (2.22 g, 0.84 mmol) using an identical procedure to that used for compound 96. Yield: 2.02 g, 91%.

### Step 9. Preparation of Conjugates 218 and 221

Conjugates 218 and 221 were prepared from compounds 128 and 114 or 118 using an identical procedure to that used for compound 1.

### Example 20a. Synthesis of Conjugates 218a and 221a

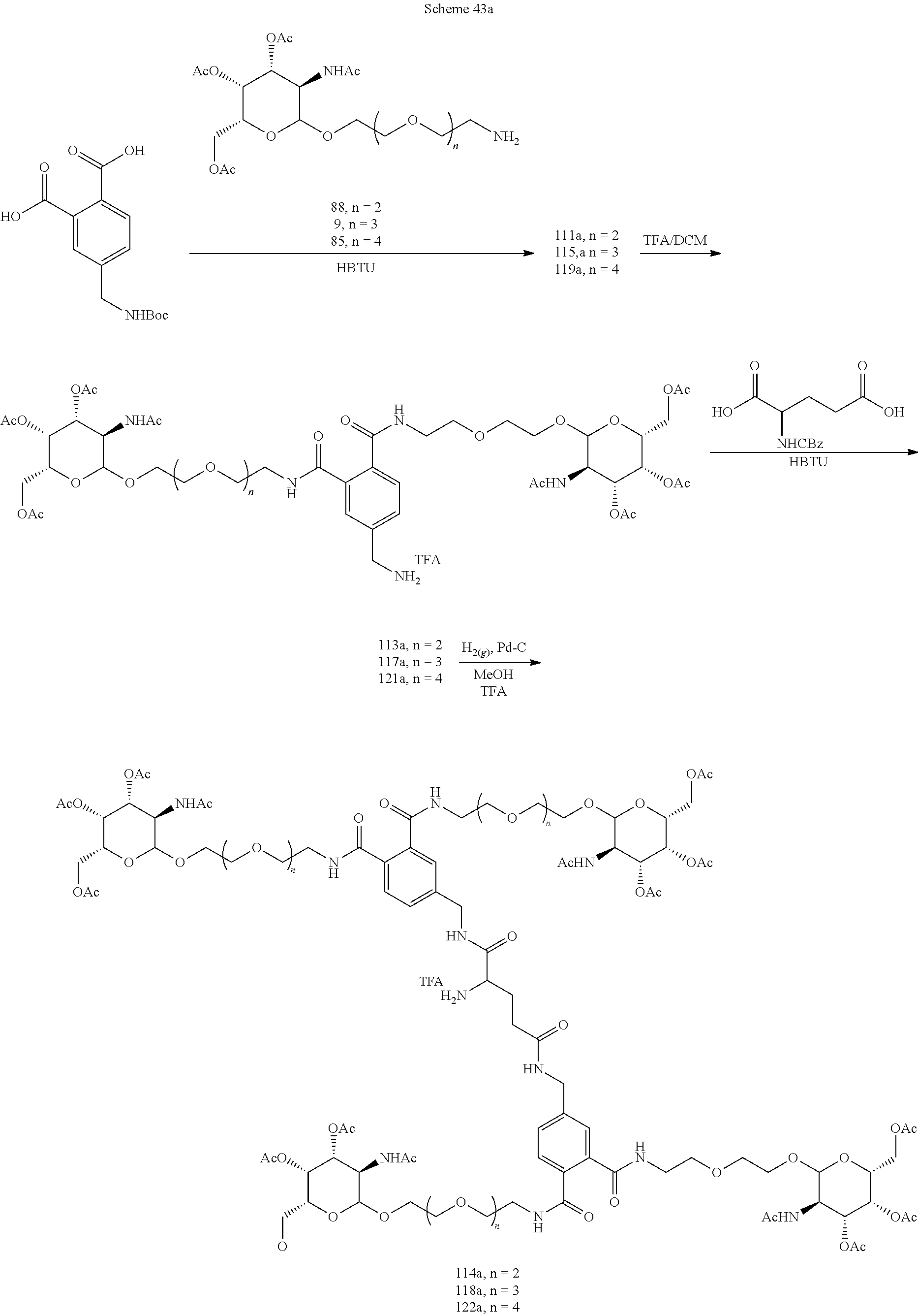

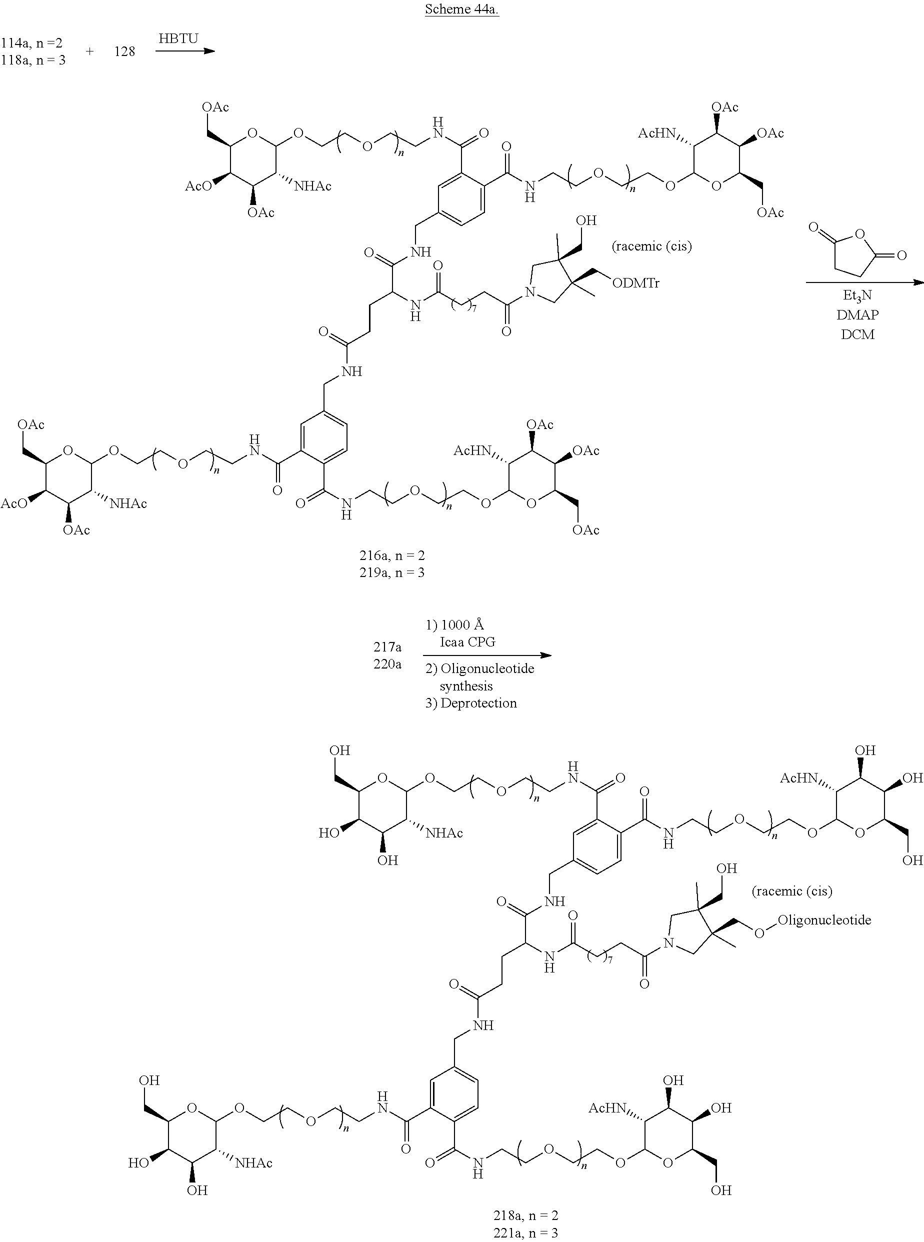

### Step 1. Preparation of Compound 111a

Compound 111a is prepared from 4-(((tert-butoxycarbonyl)amino)methyl)phthalic acid (1.13 g, 3.84 mmol) and 88 (5 g, 8.44 mmol) using an identical procedure to that used for compound 89. Yield: 2.21 g, 49%.

### Step 2. Preparation of Compound 112a

A solution of 111a (2.21 g, 1.87 mmol) in CH₂Cl₂ (40 mL) is slowly treated with TFA (5 mL). After stirring (2h) the mixture is concentrated and subjected to chromatography to yield 112a (1.08 g, 47%) as a colorless foam. Rf 0.1 (10% CH₃OH—CH₂Cl₂).

### Step 3. Preparation of Compound 113a

Compound 113a is prepared from compound 112a (1.08 g, 0.88 mmol) and (2-oxo-2-phenyl-1λ²-ethyl)-D-glutamic acid (112 mg, 0.39 mmol) using an identical procedure to that used for compound 91. Yield: 600 mg, 62%.

### Step 4. Preparation of Compound 114a

Compound 114a is prepared from compound 113a using an identical procedure to that used for compound 92.

### Step 5. Preparation of Compound 115a

Compound 115a is prepared from 4-(((tert-butoxycarbonyl)amino)methyl)phthalic acid (3.94 g, 13.3 mmol) and 9 (18.2 g, 29.4 mmol) using an identical procedure to that used for compound 93. Yield: 9.02 g, 53%.

### Step 6. Preparation of Compound 116a

Compound 116a is prepared from compound 115a (8 g, 6.3 mmol) using an identical procedure to that used for compound 11a. Yield: 3.23 g, 39%.

### Step 7. Preparation of Compound 117a

Compound 117a is prepared from compound 116a (3.23 g, 2.45 mmol) and (2-oxo-2-phenyl-1λ²-ethyl)glutamic acid (192 mg, 1.1 mmol) using an identical procedure to that used for compound 95. Yield: 2.22 g. 34%.

### Step 8. Preparation of Compound 118a

Compound 118a is prepared from compound 117a (2.22 g, 0.84 mmol) using an identical procedure to that used for compound 96. Yield: 2.02 g, 91%.

### Step 9. Preparation of Conjugates 21a8 and 221a

Conjugates 218a and 22a1 are prepared from compounds 128 and 114a or 118a using an identical procedure to that used for compound 1.

### Example 21. Synthesis of Conjugate 224

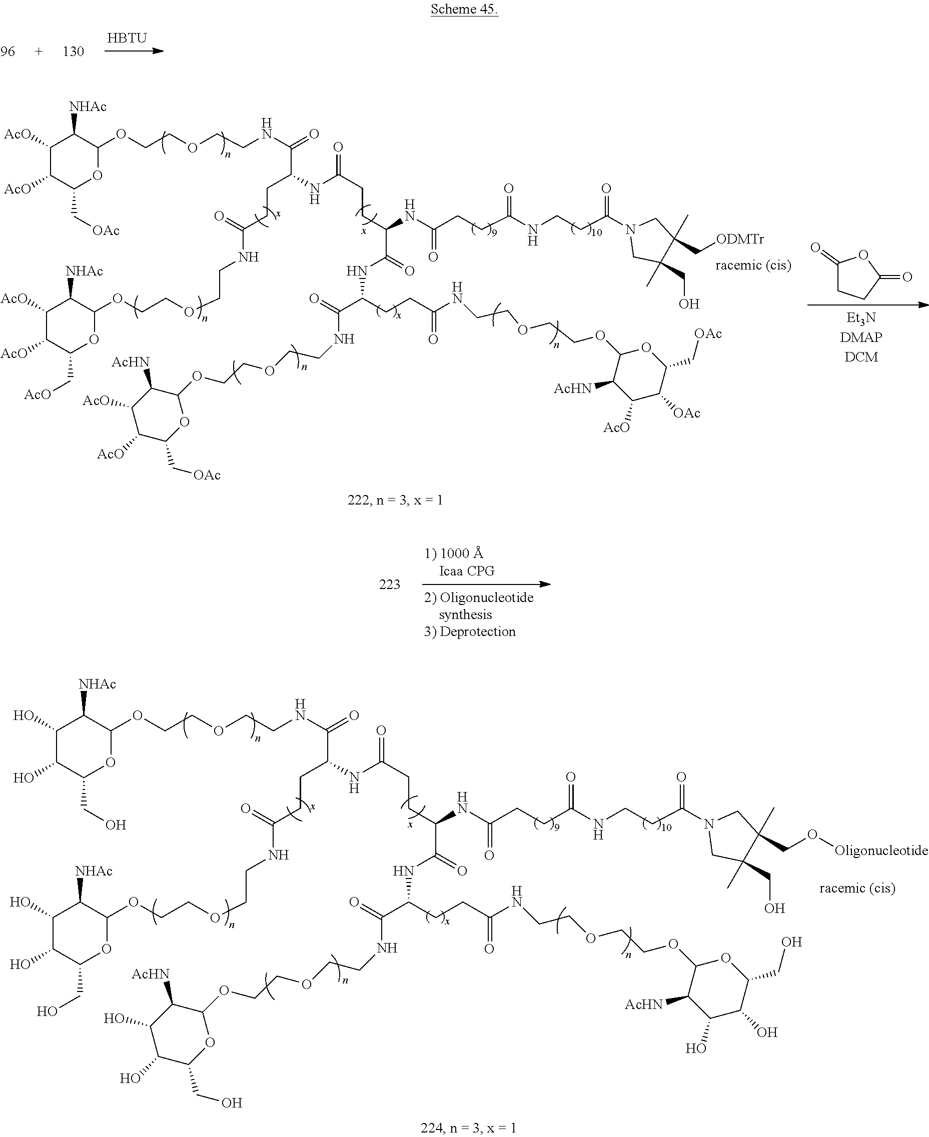

### Step 1. Preparation of Compounds 224

Conjugate 224 was prepared from compounds 96 and 130 using an identical procedure to that used for compound 1.

### Example 21a. Synthesis of Conjugate 224b

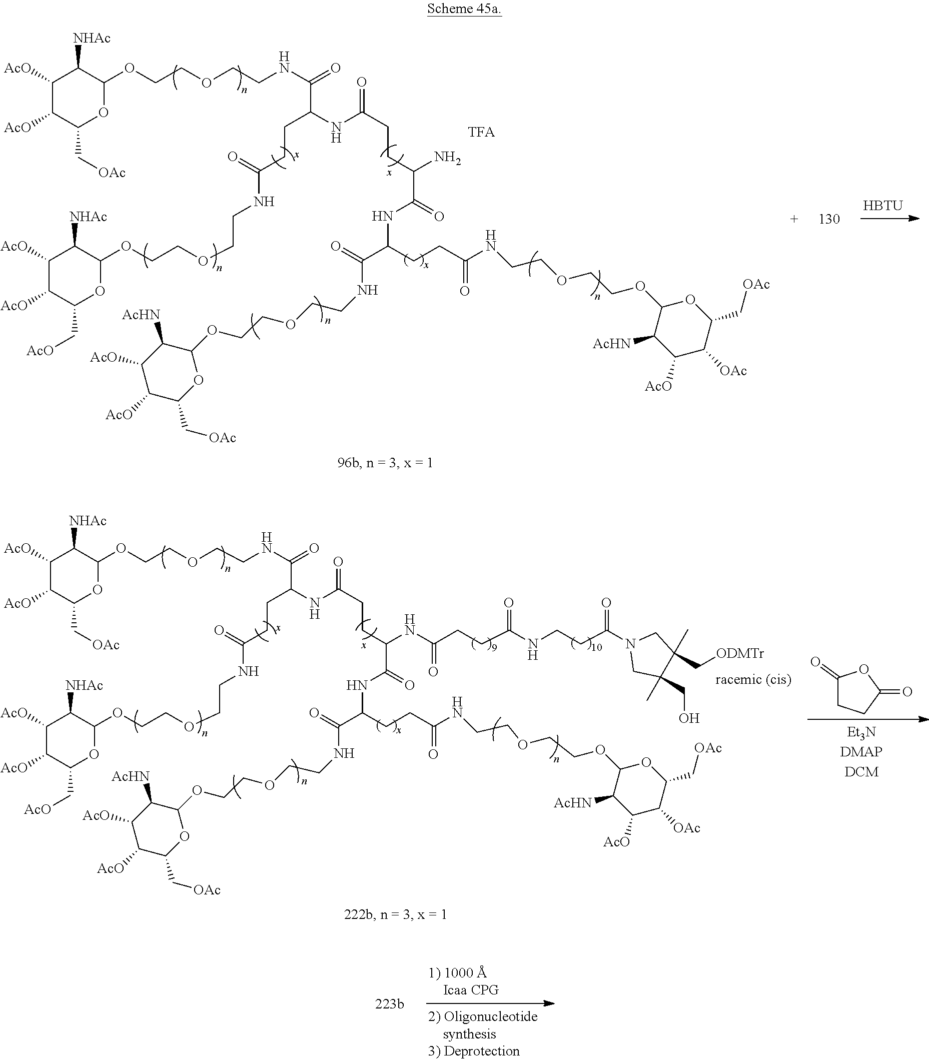

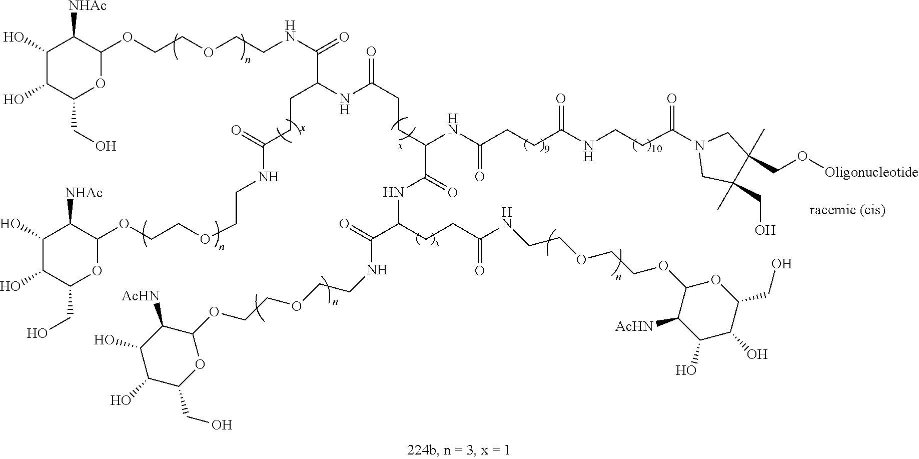

### Step 1. Preparation of Compounds 224b

Conjugate 224b is prepared from compounds 96b and 130 using an identical procedure to that used for compound 1.

### Example 22 Synthesis of Conjugate 231

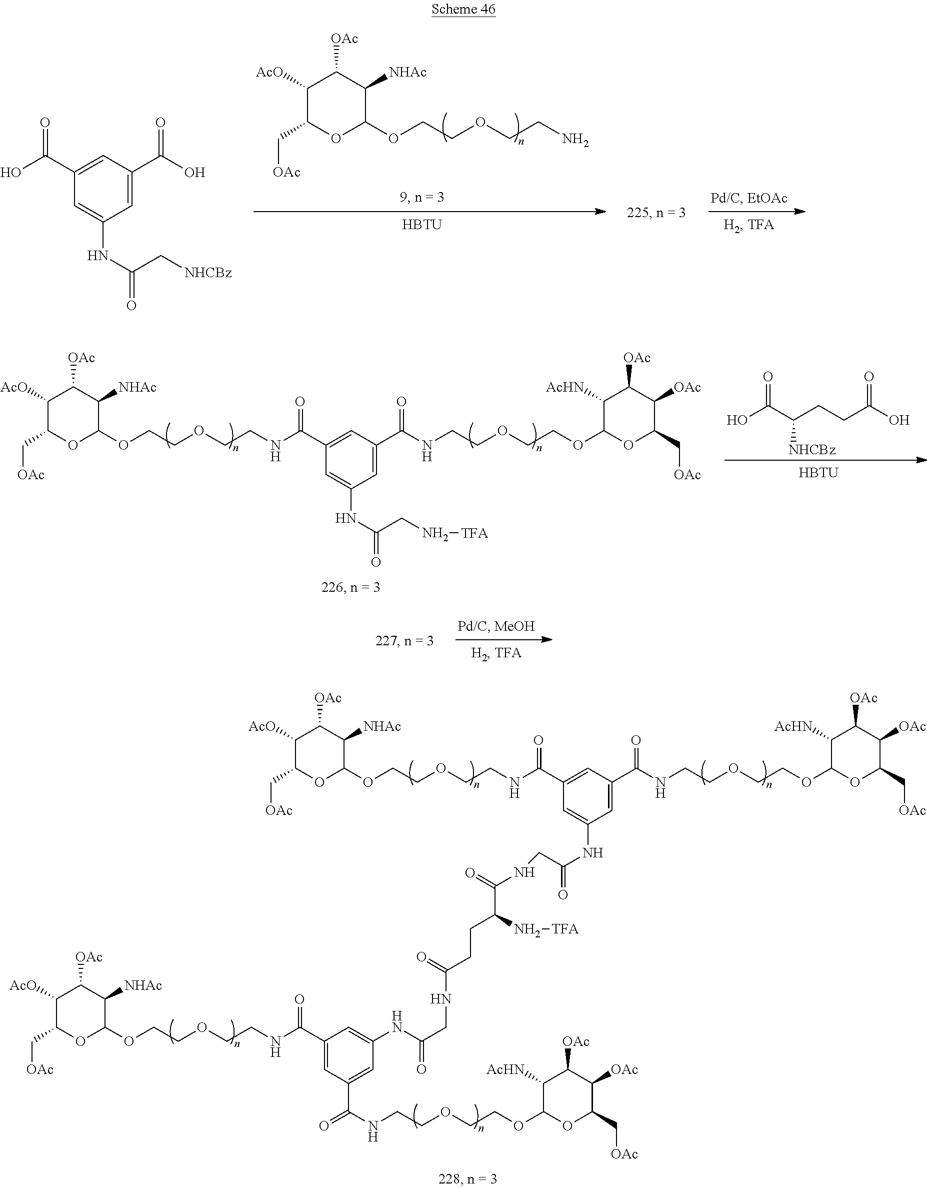

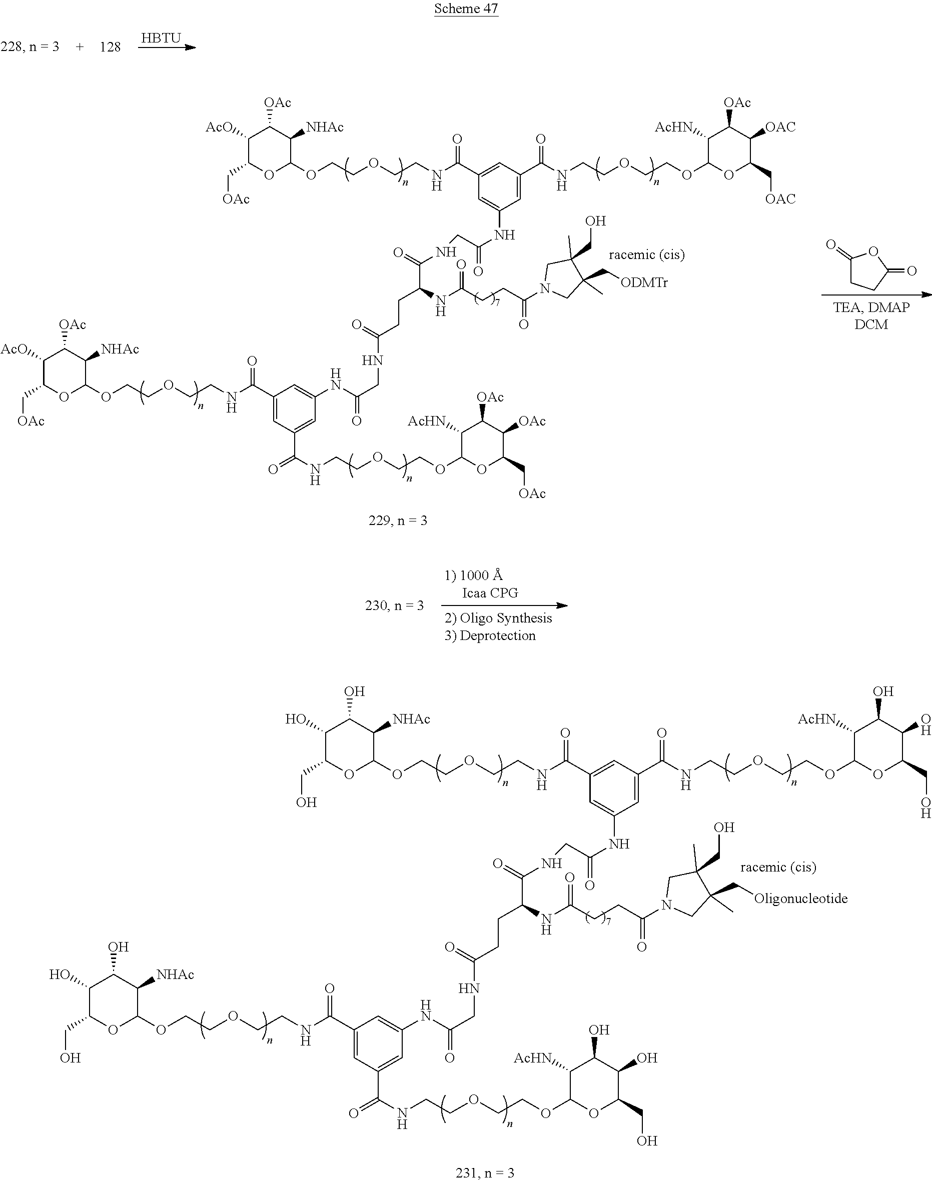

### Step 1 Preparation of Compound 225

Compound 225 was prepared from 5-(2-aminoacetamido)isophthalic acid 106 (560 mg, 1.5 mmol) and 9 (2.24 g, 3.6 mmol) using an identical procedure to that used for 89. Yield 1.6 g, 80%.

### Step 2 Preparation of Compound 226

Compound 226 was prepared in the same fashion as 14. Yield 1.22 g, 78%.

### Step 3 Preparation of Compound 227

Compound 227 was prepared in the same fashion as 89, from Z-glutamic acid (108 mg, 0.38 mmol) and 226 (1.22 g, 0.92 mmol). Yield 471 mg, 45%.

### Step 4 Preparation of Compound 228

Compound 228 was prepared in the same fashion as 14. Yield 460 mg, Quant.

### Step 5 Preparation of Compound 229

Compound 229 was prepared from 228 (460 mg, 0.17 mmol) and 128 (125 mg, 0.19 mmol) in the same fashion as 89. Yield 365 mg, 66%.

### Step 6 Preparation of Compound 231

Conjugate 231 was prepared using an identical procedure to that used for compound 1.

### Example 22a Synthesis of Conjugate 231a

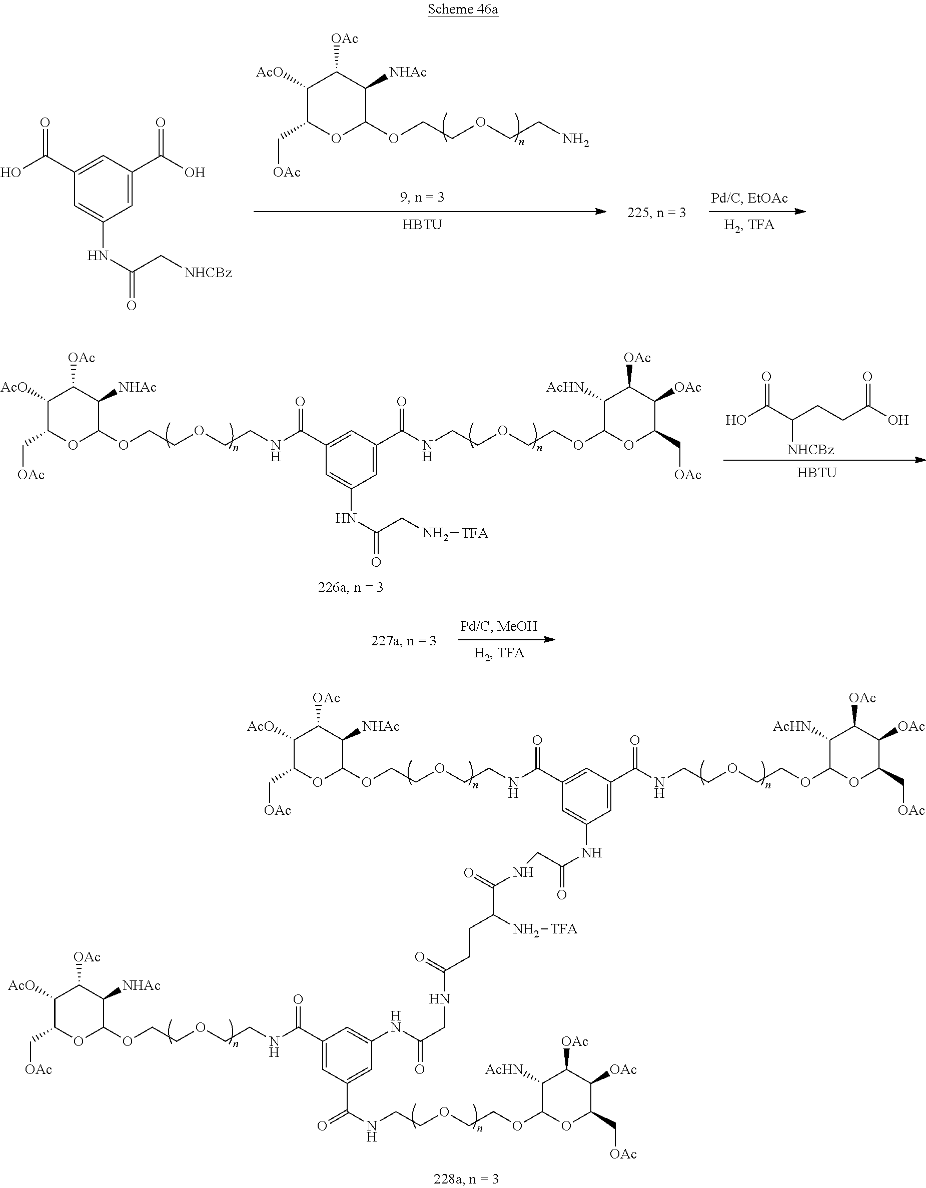

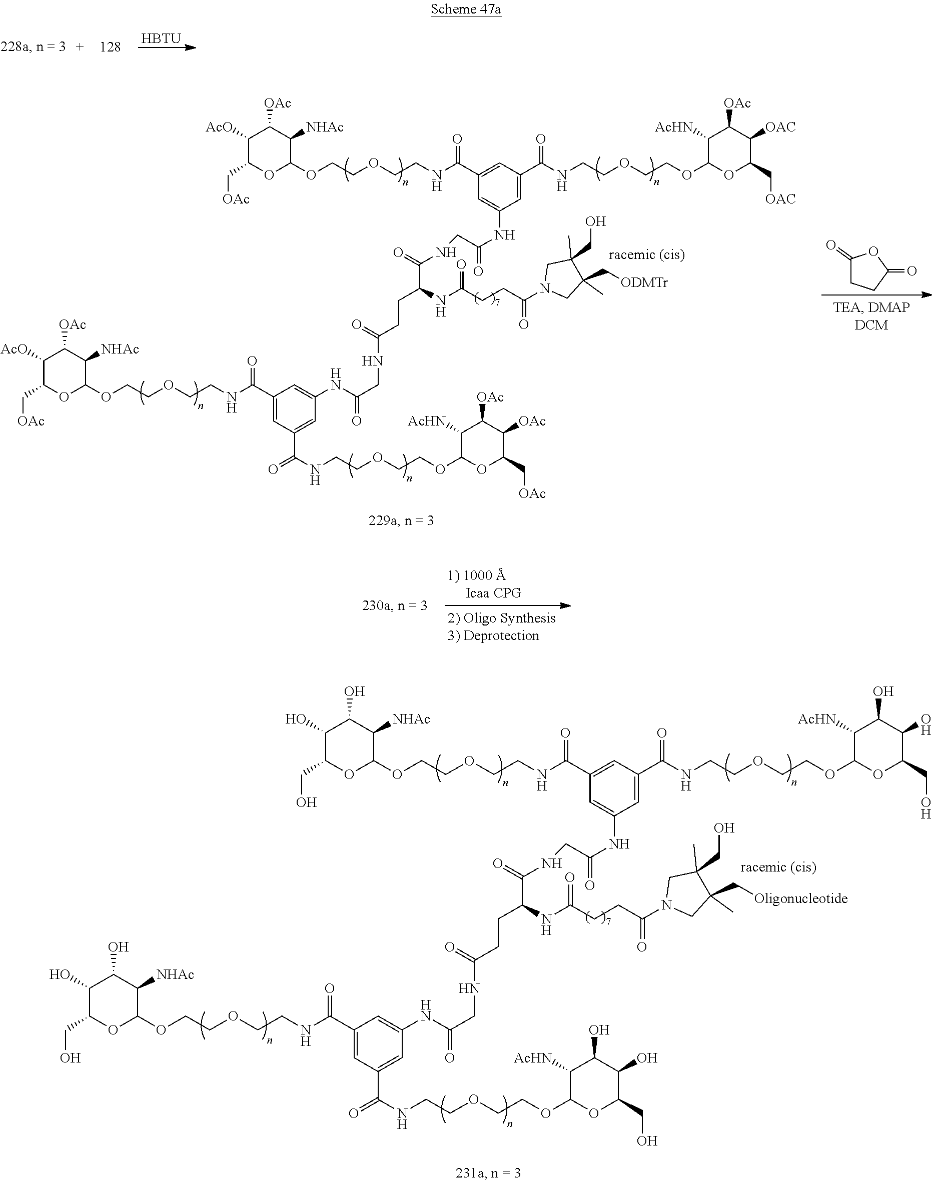

### Step 1 Preparation of Compound 225a

Compound 225a is prepared from 5-(2-aminoacetamido)isophthalic acid 106 (560 mg, 1.5 mmol) and 9 (2.24 g, 3.6 mmol) using an identical procedure to that used for 89. Yield 1.6 g, 80%.

### Step 2 Preparation of Compound 226a

Compound 226a is prepared in the same fashion as 14. Yield 1.22 g, 78%.

### Step 3 Preparation of Compound 227a

Compound 227a is prepared in the same fashion as 89, from Z-glutamic acid (108 mg, 0.38 mmol) and 226a (1.22 g, 0.92 mmol). Yield 471 mg, 45%.

### Step 4 Preparation of Compound 228a

Compound 228a is prepared in the same fashion as 14. Yield 460 mg, Quant.

### Step 5 Preparation of Compound 229a

Compound 229a is prepared from 228a (460 mg, 0.17 mmol) and 128 (125 mg, 0.19 mmol) in the same fashion as 89. Yield 365 mg, 66%.

### Step 6 Preparation of Compound 231a

Conjugate 231a is prepared using an identical procedure to that used for compound 1.

### Example 22b Synthesis of Conjugate 231b

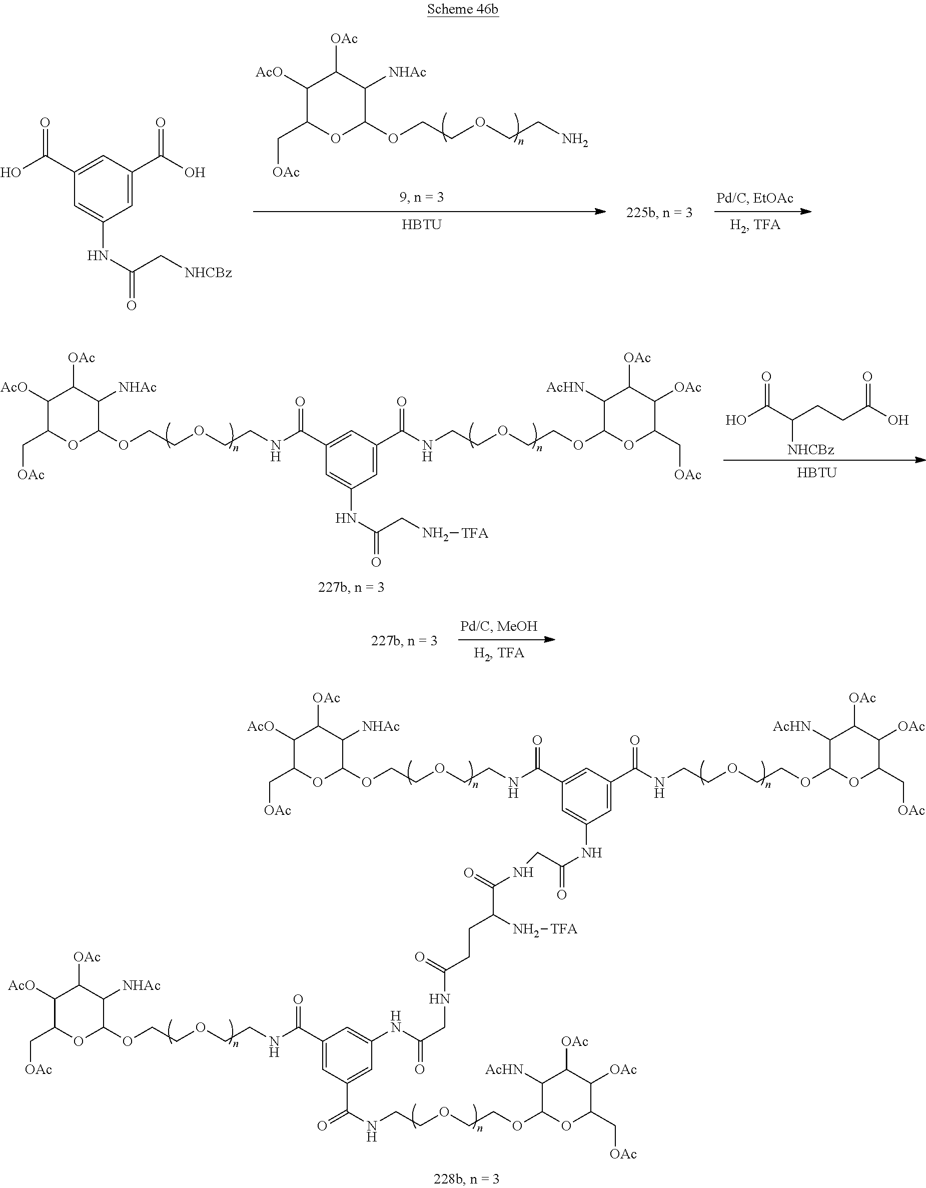

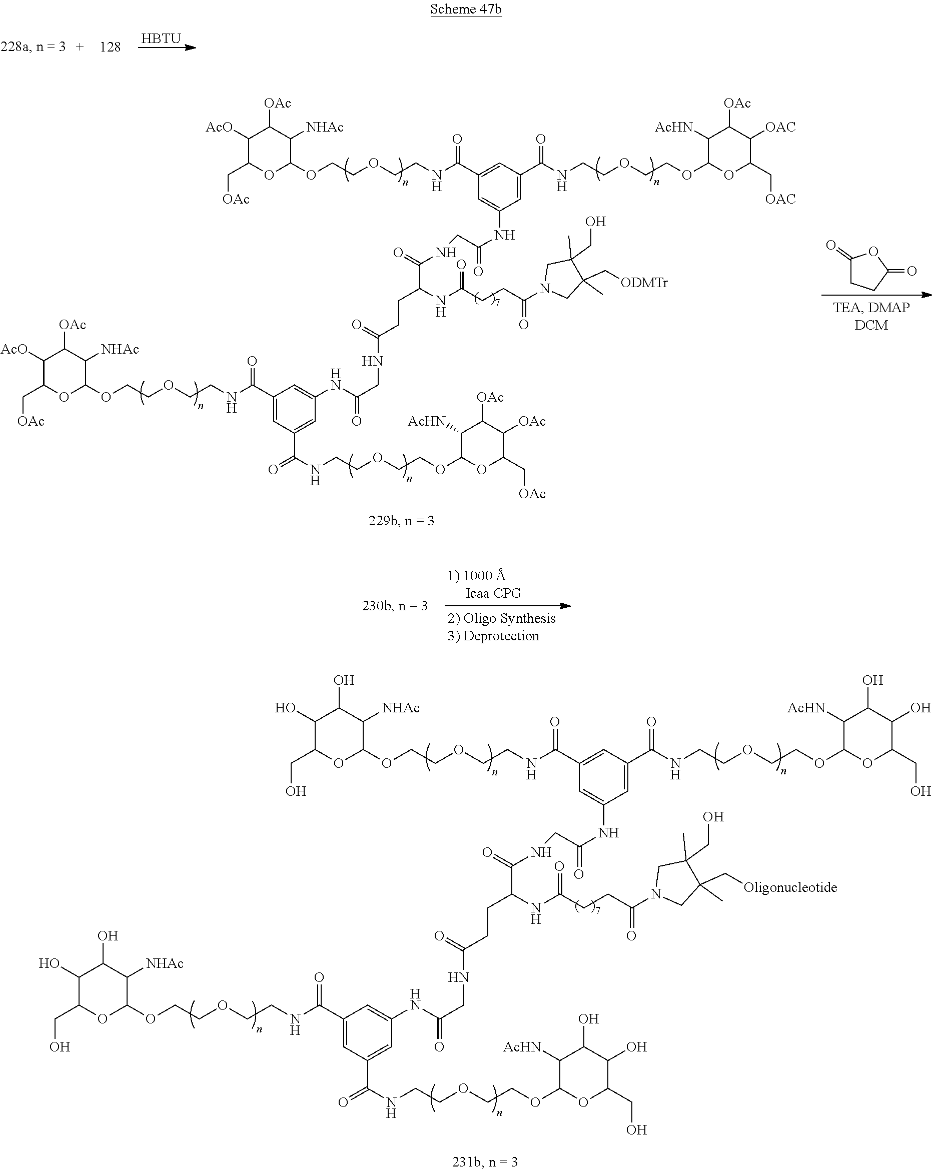

### Step 1 Preparation of Compound 225b

Compound 225b is prepared from 5-(2-aminoacetamido)isophthalic acid 106 (560 mg, 1.5 mmol) and 9 (2.24 g, 3.6 mmol) using an identical procedure to that used for 89. Yield 1.6 g, 80%.

### Step 2 Preparation of Compound 226b

Compound 226b is prepared in the same fashion as 14. Yield 1.22 g, 78%.

### Step 3 Preparation of Compound 227b

Compound 227b is prepared in the same fashion as 89, from Z-glutamic acid (108 mg, 0.38 mmol) and 226b (1.22 g, 0.92 mmol). Yield 471 mg, 45%.

### Step 4 Preparation of Compound 228b

Compound 228b is prepared in the same fashion as 14. Yield 460 mg, Quant.

### Step 5 Preparation of Compound 229b

Compound 229b is prepared from 228b (460 mg, 0.17 mmol) and 128 (125 mg, 0.19 mmol) in the same fashion as 89. Yield 365 mg, 66%.

### Step 6 Preparation of Compound 231b

Conjugate 231b is prepared using an identical procedure to that used for compound 1.

### Example 23. Synthesis of Conjugate 233

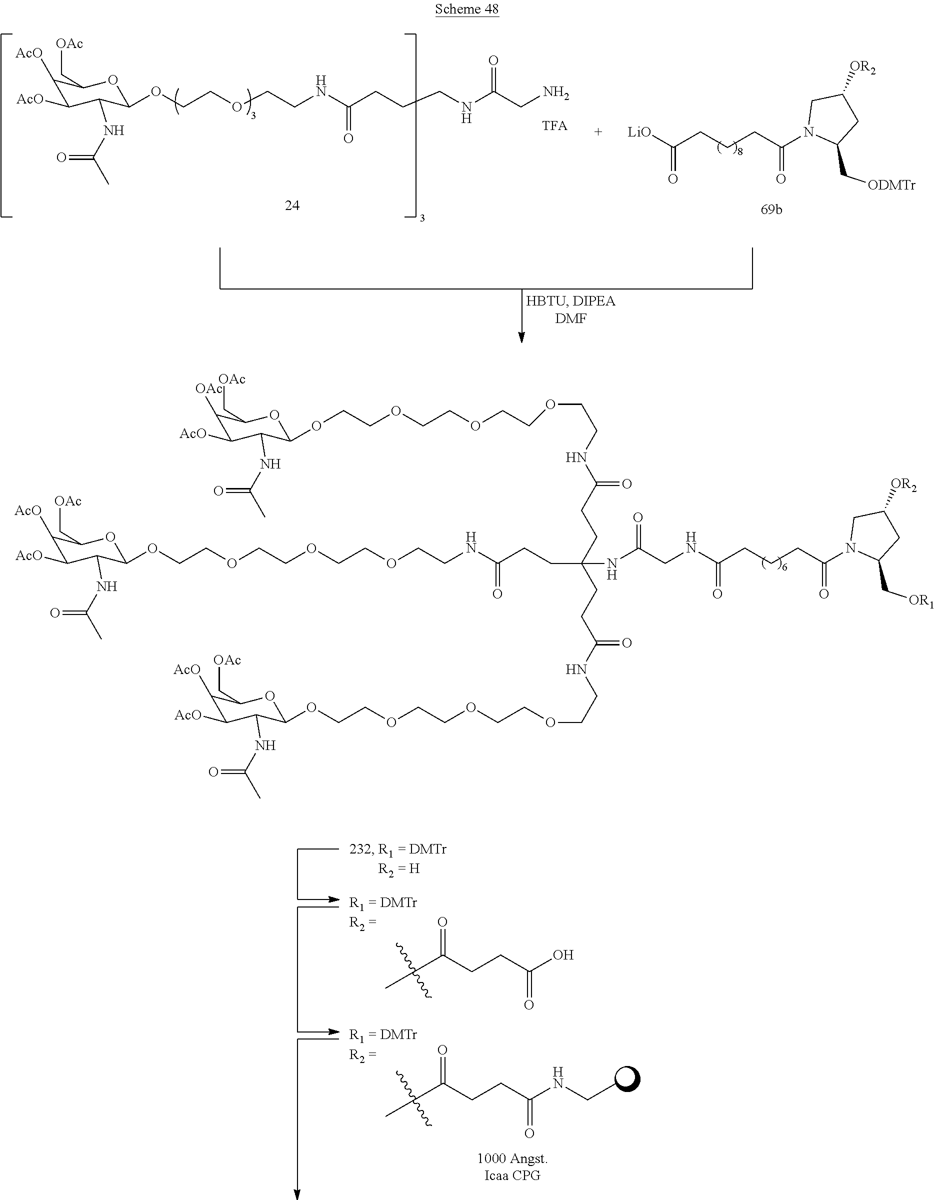

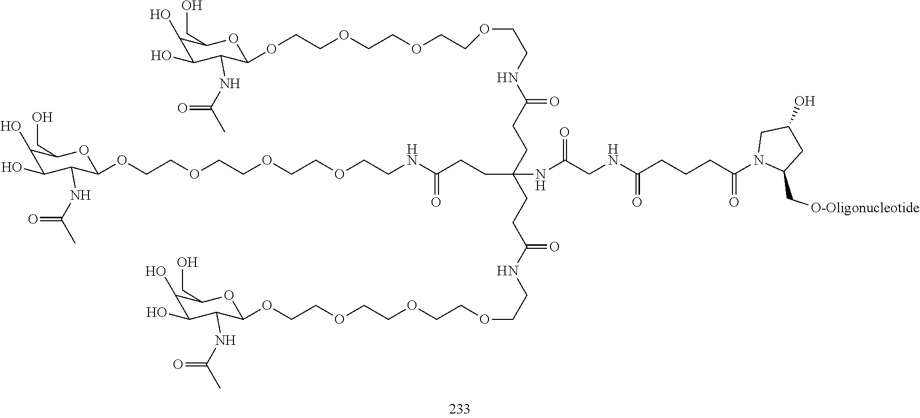

### Step 1. Preparation of Compound 232

Compound 232 was prepared from compound 24 (650 mg, 0.33 mmol) and compound 69b (175 mg, 0.33 mmol) using an identical procedure to that used for compound 19. Yield: 380 mg, 47%.

### Step 2. Preparation of Compound 233

Compound 233 was prepared from compound 232 using identical procedures to that used for compound 1.

### Example 24. Synthesis of Conjugate 235

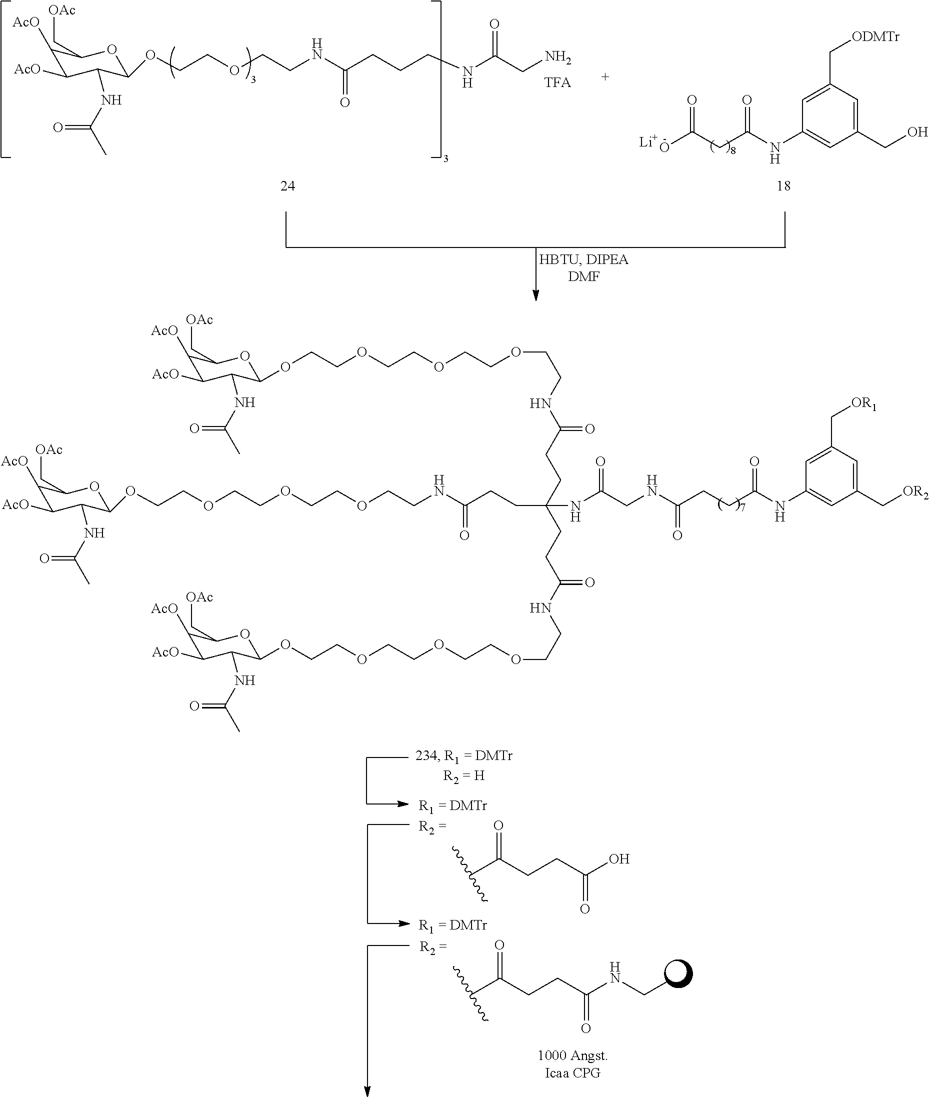

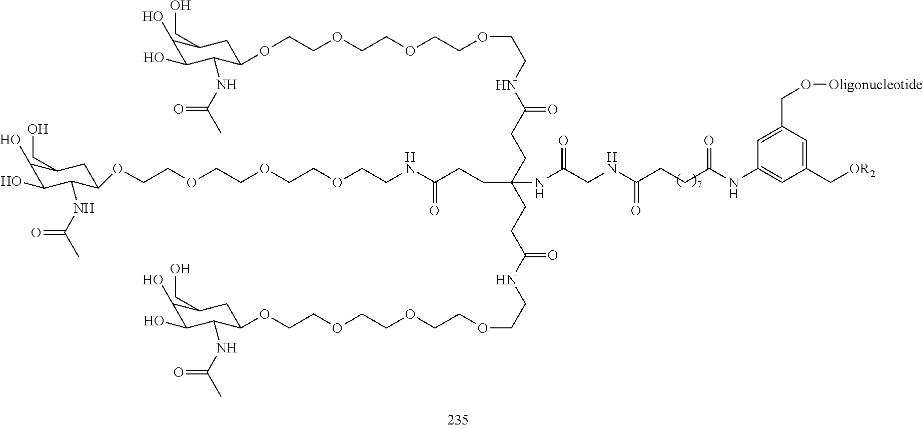

### Step 1. Preparation of Compound 234

Compound 234 was prepared from compound 24 (1.1 g, 0.55 mmol) and compound 18 (175 mg, 0.33 mmol) using an identical procedure to that used for compound 19. Yield: 685 mg, 51%.

### Step 2. Preparation of Compound 235

Compound 235 was prepared from compound 234 using identical procedures to that used for compound 1.

### Example 25 Synthesis of Conjugate 320

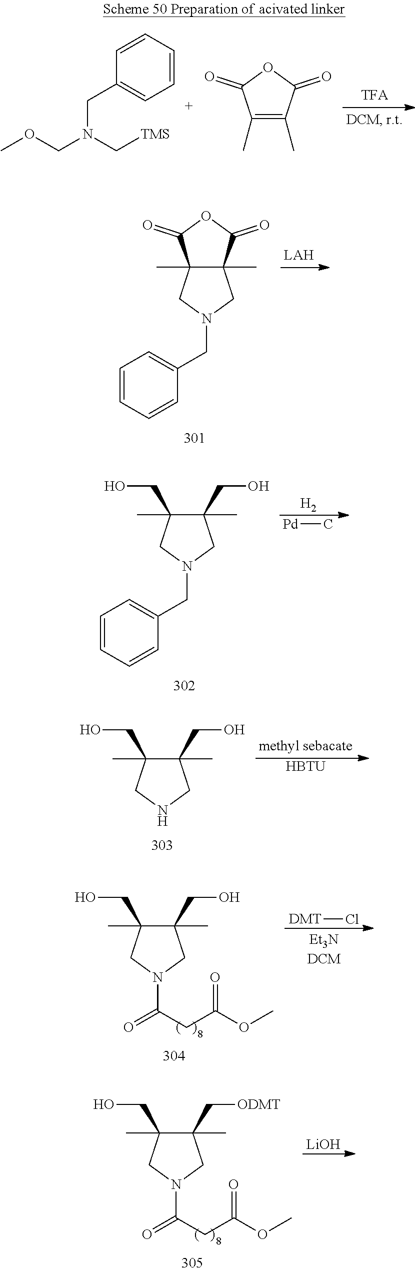

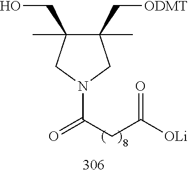

### Step 1. Preparation of Racemic (cis) 5-Benzyl-3a,6a-dimethyltetrahydro-1H-furo[3,4-c]pyrrole-1,3(3aH)-dione 301

To a cooled solution (0° C.) of 3,4-dimethylfuran-2,5-dione (3 g, 24 mmol) and N-benzyl-1-methoxy-N-((trimethylsilyl)methyl)methanamine (7 g, 29.8 mmol) in dichloromethane (75 mL) was slowly added trifluoroacetic acid (75 μL). Stir overnight allowing the solution to slowly warm to room temperature as the ice bath melted. The reaction mixture was concentrated to dryness, dissolved in ethyl acetate (100 mL), washed with saturated sodium bicarbonate (2×100 mL), dried on magnesium sulfate, filtered and concentrated to dryness. Purification by column chromatography on silica gel (gradient: 20% ethyl acetate in hexanes to 100% ethyl acetate) afforded (3aR,6aS)-5-Benzyl-3a,6a-dimethyltetrahydro-1H-furo[3,4-c]pyrrole-1,3(3aH)-dione as a yellow oil (3.5 g, 56%).

### Step 2. Preparation of Racemic (cis) (1-Benzyl-3,4-dimethylpyrrolidine-3,4-diyl)dimethanol 302

To a cooled (0° C.) solution of (3aR,6aS)-5-Benzyl-3a,6a-dimethyltetrahydro-1H-furo[3,4-c]pyrrole-1,3(3aH)-dione (3.5 g, 13.4 mmol) in anhydrous diethyl ether (50 mL) was added slowly lithium aluminum hydride pellets (1.5 g, 40 mmol) over three portions. The solution was stirred overnight warming to room temperature as the ice water bath melted. Upon completion, the reaction was cooled to 0° C. and very slowly quenched with 1.5 mL of 5M NaOH followed by 1.5 mL of water. Stir for 30 minutes then add magnesium sulfate and filter. The filtrate was concentrated to afford ((3R,4S)-1-Benzyl-3,4-dimethylpyrrolidine-3,4-diyl)dimethanol as a colorless oil (2.7 g).

### Step 3. Preparation of Racemic (cis) (3,4-Dimethylpyrrolidine-3,4-diyl)dimethanol 303

To a solution of ((3R,4S)-1-Benzyl-3,4-dimethylpyrrolidine-3,4-diyl)dimethanol (10 g, 40 mmol) in methanol (10 mL) was added 10% palladium on activated charcoal wet (1 g). The solution was stirred vigorously under a hydrogen atmosphere for 16 hours. Upon completion the solution was filtered through Celite, and concentrated to dryness to afford ((3R,4S)-3,4-Dimethylpyrrolidine-3,4-diyl)dimethanol as a colorless solid (5.5 g, 86%).

### Step 4. Preparation of Racemic (cis) Methyl 10-(3,4-bis(hydroxymethyl)-3,4-dimethylpyrrolidin-1-yl)-10-oxodecanoate 304

A solution of 3 (1.3 g, 8.2 mmol) and monomethyl sebacate (1.8 g, 8.2 mmol) in CH₂Cl₂ (100 mL) was treated with HBTU (3.41 g, 9.02 mmol) and Hunig's base (5.71 mL, 32.8 mmol). After stirring overnight the mixture was washed with NaHCO₃ (sat. aq.), water and brine, then dried (MgSO₄), filtered and concentrated. The crude material was subjected to chromatography (gradient: 0% CH₃OH—CH₂Cl₂ to 20%) to yield 4 (1.8 g, 61%).

### Step 5. Preparation of Racemic (cis) Methyl 10-(3-((bis(4-methoxyphenyl)(phenyl)-methoxy)methyl)-4-(hydroxymethyl)-3,4-dimethylpyrrolidin-1-yl)-10-oxodecanoate 305

A solution of 304 (1.8 g, 5.0 mmol) and 4,4′-Dimethoxytrityl chloride (1.7 g, 5.0 mmol) in pyridine (180 mL) was stirred overnight. The pyridine was then removed under reduced pressure and the crude material was subjected to chromatography (gradient: 0% CH₃OH—CH₂Cl₂ to 10%) to yield 5 (1.4 g, 42%) as a yellow oil.

### Step 6. Preparation of Racemic (cis) Lithium 10-(3-((bis(4-methoxyphenyl)-(phenyl)methoxy)methyl)-4-(hydroxymethyl)-3,4-dimethylpyrrolidin-1-yl)-10-oxodecanoate 306

To a solution of compound 305 (3.0 g, 4.6 mmol) in THF (50 mL) and water (50 mL) was added lithium hydroxide (121 mg, 5.0 mmol). The solution was stirred for 4 hours at room temperature then concentrated to remove the THF. The remaining aqueous solution was freeze dried overnight to afford a pale pink solid (2.9 g, quantitative). Compound 306 was prepared as a mixture of two cis-diastereomers.

### Scheme 51 Synthesis of peracetylated galactosamine 307

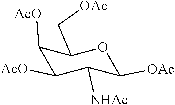 [SMILES: CC(=O)NC1C(OC(C)=O)OC(COC(C)=O)C(OC(C)=O)C1OC(C)=O]

D-Galactosamine hydrochloride (250 g, 1.16 mol) in pyridine (1.5 L) was treated with acetic anhydride (1.25 L, 13.2 mol) over 45 minutes. After stirring overnight the reaction mixture was divided into three 1 L portions. Each 1 L portion was poured into 3 L of ice water and mixed for one hour. After mixing the solids were filtered off, combined, frozen over liquid nitrogen and then lyophilized for five days to yield peracetylated galactosamine 7 (369.4 g, 82%) as a white solid. Rf (0.58, 10% MeOH—CH₂Cl₂).

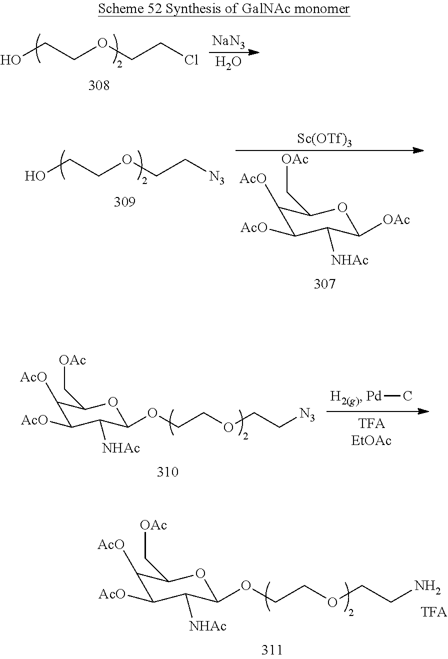

### Step 1 Preparation of Compound 309

A solution of 2-[2-(2-chloroethoxy)]ethanol 308 (100 g, 593 mmol) in water (1 L) was treated with NaN₃ (77 g, 1.19 mol) and heated (90° C.). After stirring (72 hours) the solution was cooled (RT) and extracted (4×) with CH₂Cl₂. The combined organics were washed with brine, dried (MgSO4), filtered, concentrated and used without further processing. Compound 9 (88.9 g, 86%) was obtained as a pale yellow oil.

### Step 2 Preparation of Compound 310

A solution of 7 (2.76 g, 7.1 mmol) and 309 (1.37 g, 7.8 mmol) in 1,2-dichloroethane (40 mL) was treated with Sc(OTf)₃ (174 mg, 0.36 mmol) and heated (85° C.). After stirring (2 hours) the mixture was cooled (RT) and quenched by the addition of TEA (4 mL) and concentrated. The crude material was subjected to chromatography to yield 310 (3.03 g, 85%) as a pale yellow foam.

### Step 3 Preparation of Compound 311

A solution of 310 (3.02 g, 5.99 mmol) and Pd/C (300 mg, 10% Pd loading—wet support) in EtOAc (30 mL) was treated with TFA (576 μL, 7.5 mmol). The reaction mixture was purged with hydrogen gas (45 min) then purged with nitrogen gas (10 min), then filtered through celite. The filtrate was concentrated and then subjected to chromatography to yield 311 (2.67 g, 75%) as a brown foam.

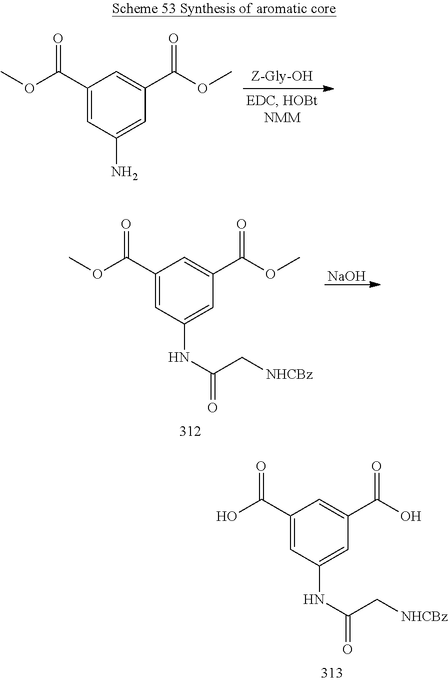

### Step 1. Preparation of Dimethyl 5-(2-((2-oxo-2-phenyl-1λ²-ethyl)amino)acetamido)-isophthalate 312

A solution of dimethyl 5-aminoisophthalate (5 g, 24 mmol), Z-Gly-OH (5 g, 24 mmol), EDC (5 g, 26.3 mmol), HOBt (3.6 g, 26.3 mmol), NMM (2.9 mL, 26.3 mmol) in DMF (50 mL) was stirred overnight at room temperature. Upon completion, the reaction mixture was diluted with ethyl acetate (250 mL) and washed with each 1M HCl (2×100 mL), saturated sodium bicarbonate (1×100 mL) and brine (2×100 mL). Dry on magnesium sulfate, filter and concentrate to dryness to afford Dimethyl 5-(2-((2-oxo-2-phenyl-1λ²-ethyl)amino)-acetamido)isophthalate as a colorless solid (7.2 g, 79%).

### Step 2. Preparation of 5-(2-((2-oxo-2-phenyl-1λ²-ethyl)amino)acetamido)isophthalic acid 313

To a solution of methyl 5-(2-((2-oxo-2-phenyl-1λ²-ethyl)amino)acetamido)isophthalate (7.2 g) in methanol (25 mL) and THF (25 mL) was added 1M NaOH (25 mL). The solution was stirred at room temperature for 2 hours then concentrated to remove THF and MeOH. The aqueous solution remaining was diluted with water (75 mL), cooled on an ice water bath and acidified to pH=1 with 6M HCl. The solid was filtered and washed with water (3×100 mL). The solid was freeze dried to afford 5-(2-((2-oxo-2-phenyl-1λ²-ethyl)amino)acetamido)-isophthalic acid (6.9 g, quantitative).

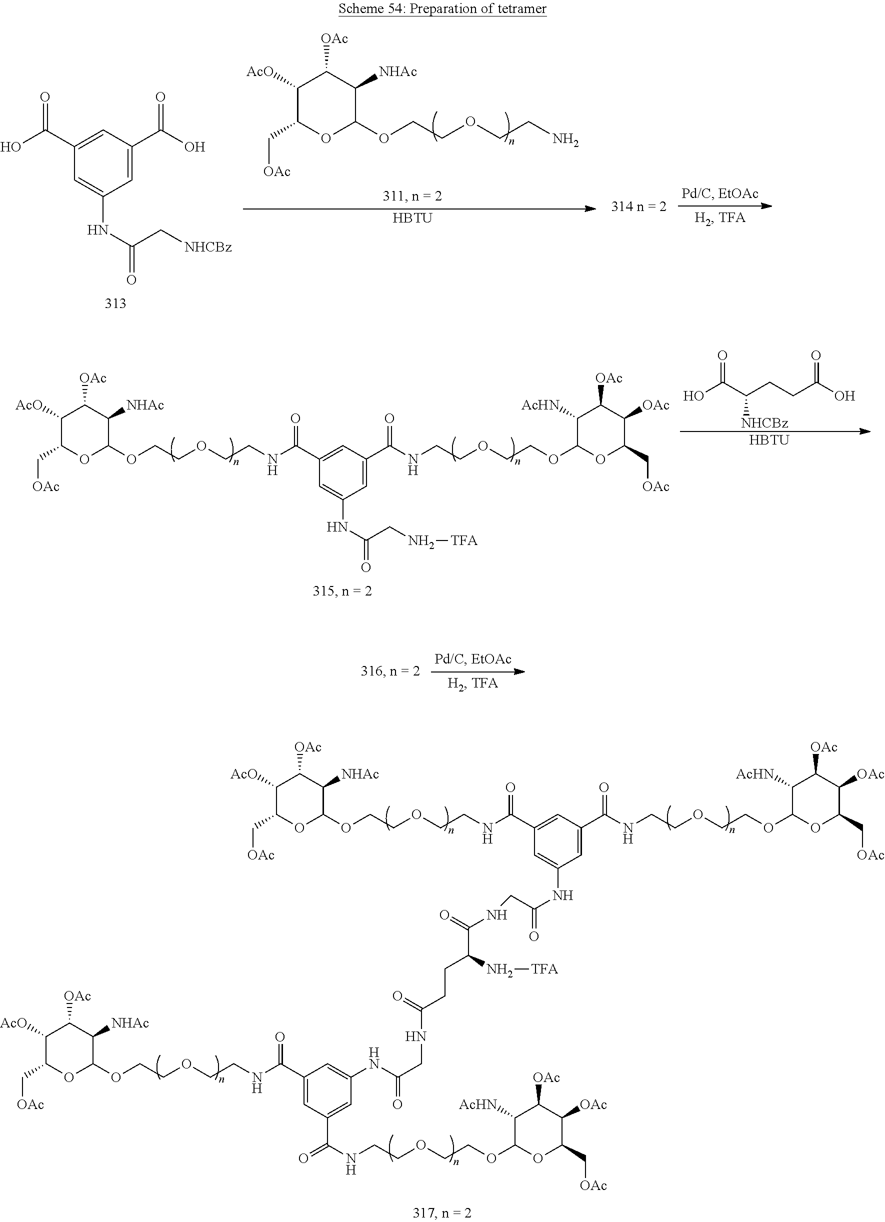

### Step 1 Preparation of Compound 314

A solution of 313 (2.09 g, 5.6 mmol) and 311 (8.34 g, 14.07 mmol) in CH₂Cl₂ (150 mL) was treated with HBTU (6.4 g, 16.9 mmol) and Hunig's base (7.35 mL, 42.2 mmol). After stirring (overnight) the reaction mixture was poured into NaHCO₃ (sat. aq.) then washed with water and brine, dried (MgSO₄), filtered and concentrated. The crude material was subjected to chromatography (gradient 1-12% CH₃OH—CH₂Cl₂) to yield 6 (3.97 g, 55%) as a pale yellow foam.

### Step 2 Preparation of Compound 315

Compound 314 (3.92 g. 3.07 mmol), Pd/C (400 mg. 10% loading—wet support) and trifluoroacetic acid (308 μL, 4 mmol) was purged with H₂. After stirring under H₂ (overnight), the mixture was purged with N₂ (15-20 min) then filtered through celite and concentrated. The crude material was subjected to chromatography to yield 7 (3.36 g, 86%) as a white to cream colored foam.

### Step 3 Preparation of Compound 316

Compound 316 was prepared in the same fashion as 314, from Z-glutamic acid (306 mg, 1.09 mmol) and 315 (3.3 g, 2.6 mmol). Yield 1.66 g, 60%.

### Step 4 Preparation of Compound 317

Compound 317 was prepared in the same fashion as 315. Yield 1.65 g, Quant.

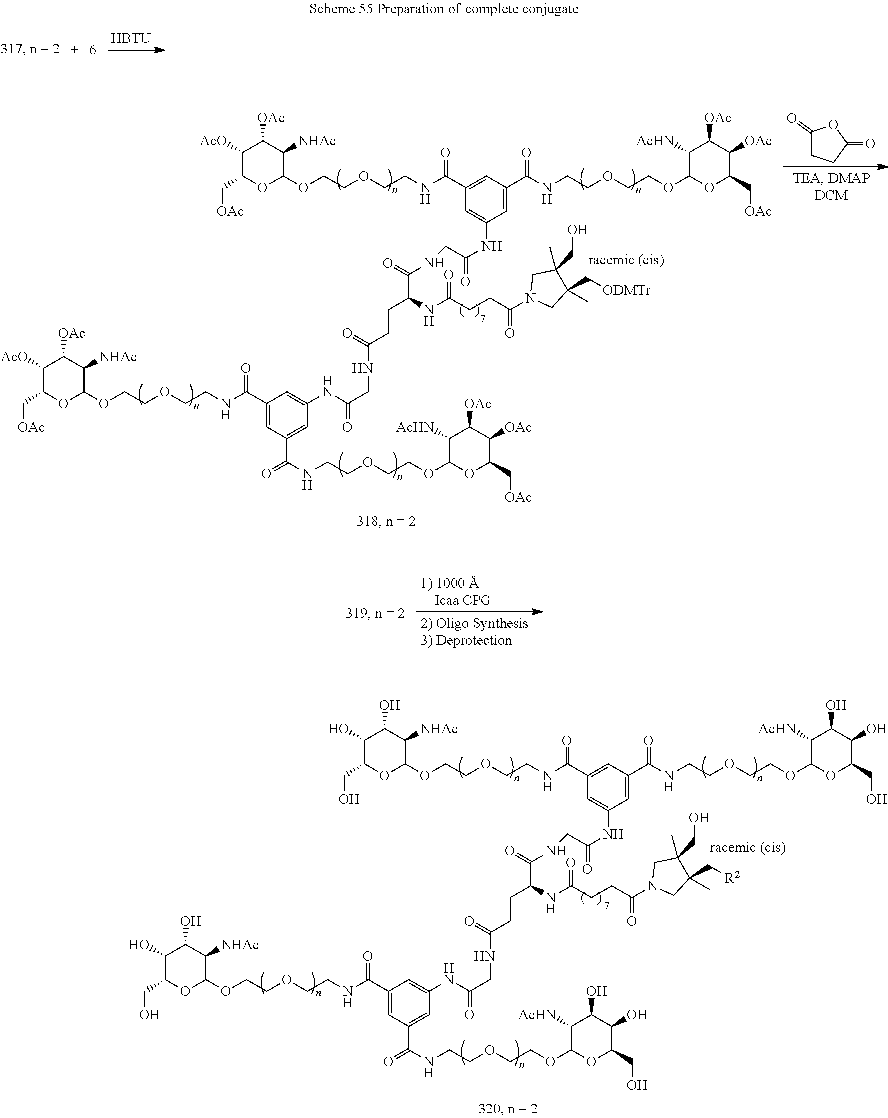

### Step 1 Preparation of Compound 318

A solution of 317 (1.91 g, 0.75 mmol) in CH₂Cl₂ (100 mL) was treated first with Hunig's base (392 μL, 2.25 mmol) then 6 (a mixture of two cis-diastereomers, 509 mg, 0.79 mmol) followed by HBTU (356 mg, 0.94 mmol). After stirring (overnight) the solution was poured into NaHCO₃ (sat. aq.) then washed with water and brine, dried (MgSO4), filtered and concentrated. The crude material was subjected to chromatography to yield 318 (1.19 g, 52%) as a white foam.

### Step 2 Preparation of Compound 319

A solution of 318 (1.19 g, 0.39 mmol) in 1,2 dichloroethane (100 mL) was treated with TEA (542 μL, 3.9 mmol), DMAP (238 mg, 1.95 mmol) and succinic anhydride (195 mg, 1.95 mmol) and heated (85° C.). After stirring (2.5 hours) the solution was removed from heat and treated with CH₃OH (10 mL) and allowed to stir (1 hour). After stirring the mixture was poured into NaHCO₃ (sat. aq.) then washed with brine, dried (MgSO4), filtered and concentrated. The residue obtained was used without further processing. Yield=1.4 g, Quant.

### Step 3 Preparation of conjugate 320

The succinate 319 was loaded onto 1000 Å LCAA (long chain aminoalkyl) CPG (control pore glass) using standard amide coupling chemistry. A solution of diisopropylcarbodiimide (52.6 μmol), N-hydroxy succinimide (0.3 mg, 2.6 μmol) and pyridine (10 μL) in anhydrous acetonitrile (0.3 mL) was added to 319 (20.6 mg, 8 μmol) in anhydrous dichloromethane (0.2 mL). This mixture was added to LCAA CPG (183 mg). The suspension was gently mixed overnight at room temperature. Upon disappearance of 319 (HPLC), the reaction mixture was filtered and the CPG was washed with 1 mL of each dichloromethane, acetonitrile, a solution of 5% acetic anhydride/5% N-methylimidazole/5% pyridine in THF, then THF, acetonitrile and dichloromethane. The CPG was then dried overnight under high vacuum. Loading was determined by standard DMTr assay by UV/Vis (504 nm) to be 19 μmol/g. The resulting GalNAc loaded CPG solid support was employed in automated oligonucleotide synthesis using standard procedures. Nucleotide deprotection followed by removal from the solid support (with concurrent galactosamine acetate deprotection) afforded the GalNAc-oligonucleotide conjugate 320.

### Example 26 Synthesis of Conjugate 520

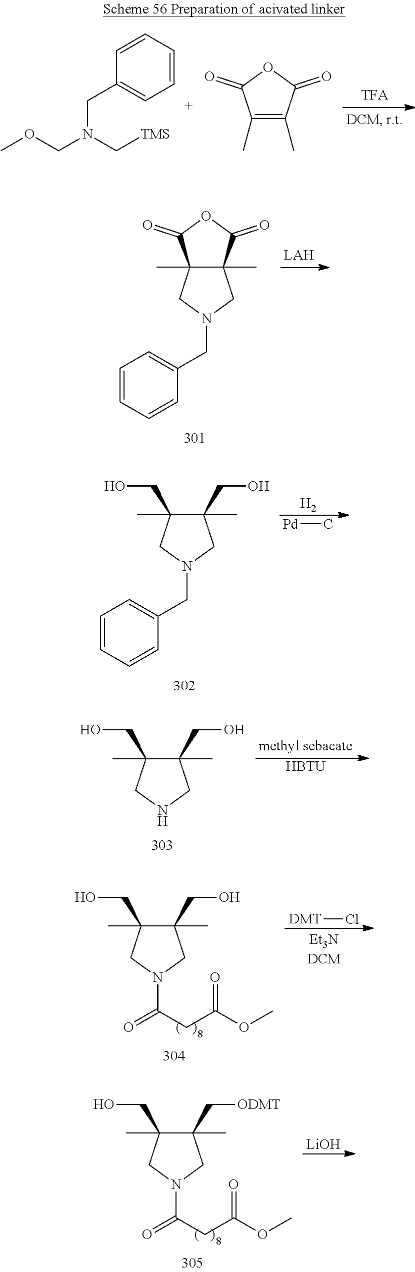

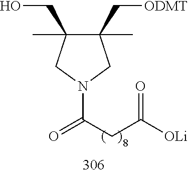

### Step 1. Preparation of Racemic (cis) 5-Benzyl-3a,6a-dimethyltetrahydro-1H-furo[3,4-c]pyrrole-1,3(3aH)-dione 301

To a cooled solution (0° C.) of 3,4-dimethylfuran-2,5-dione (3 g, 24 mmol) and N-benzyl-1-methoxy-N-((trimethylsilyl)methyl)methanamine (7 g, 29.8 mmol) in dichloromethane (75 mL) was slowly added trifluoroacetic acid (75 μL). Stir overnight allowing the solution to slowly warm to room temperature as the ice bath melted. The reaction mixture was concentrated to dryness, dissolved in ethyl acetate (100 mL), washed with saturated sodium bicarbonate (2×100 mL), dried on magnesium sulfate, filtered and concentrated to dryness. Purification by column chromatography on silica gel (gradient: 20% ethyl acetate in hexanes to 100% ethyl acetate) afforded (3aR,6aS)-5-Benzyl-3a,6a-dimethyltetrahydro-1H-furo[3,4-c]pyrrole-1,3(3aH)-dione as a yellow oil (3.5 g, 56%).

### Step 2. Preparation of Racemic (cis) (1-Benzyl-3,4-dimethylpyrrolidine-3,4-diyl)dimethanol 302

To a cooled (0° C.) solution of (3aR,6aS)-5-Benzyl-3a,6a-dimethyltetrahydro-1H-furo[3,4-c]pyrrole-1,3(3aH)-dione (3.5 g, 13.4 mmol) in anhydrous diethyl ether (50 mL) was added slowly lithium aluminum hydride pellets (1.5 g, 40 mmol) over three portions. The solution was stirred overnight warming to room temperature as the ice water bath melted. Upon completion, the reaction was cooled to 0° C. and very slowly quenched with 1.5 mL of 5M NaOH followed by 1.5 mL of water. Stir for 30 minutes then add magnesium sulfate and filter. The filtrate was concentrated to afford ((3R,4S)-1-Benzyl-3,4-dimethylpyrrolidine-3,4-diyl)dimethanol as a colorless oil (2.7 g).

### Step 3. Preparation of Racemic (cis) (3,4-Dimethylpyrrolidine-3,4-diyl)dimethanol 303

To a solution of ((3R,4S)-1-Benzyl-3,4-dimethylpyrrolidine-3,4-diyl)dimethanol (10 g, 40 mmol) in methanol (10 mL) was added 10% palladium on activated charcoal wet (1 g). The solution was stirred vigorously under a hydrogen atmosphere for 16 hours. Upon completion the solution was filtered through Celite, and concentrated to dryness to afford ((3R,4S)-3,4-Dimethylpyrrolidine-3,4-diyl)dimethanol as a colorless solid (5.5 g, 86%).

### Step 4. Preparation of Racemic (cis) Methyl 10-(3,4-bis(hydroxymethyl)-3,4-dimethylpyrrolidin-1-yl)-10-oxodecanoate 304

A solution of 3 (1.3 g, 8.2 mmol) and monomethyl sebacate (1.8 g, 8.2 mmol) in CH₂Cl₂ (100 mL) was treated with HBTU (3.41 g, 9.02 mmol) and Hunig's base (5.71 mL, 32.8 mmol). After stirring overnight the mixture was washed with NaHCO₃ (sat. aq.), water and brine, then dried (MgSO₄), filtered and concentrated. The crude material was subjected to chromatography (gradient: 0% CH₃OH—CH₂Cl₂ to 20%) to yield 4 (1.8 g, 61%).

### Step 5. Preparation of Racemic (cis) Methyl 10-(3-((bis(4-methoxyphenyl)(phenyl)-methoxy)methyl)-4-(hydroxymethyl)-3,4-dimethylpyrrolidin-1-yl)-10-oxodecanoate 305

A solution of 304 (1.8 g, 5.0 mmol) and 4,4′-Dimethoxytrityl chloride (1.7 g, 5.0 mmol) in pyridine (180 mL) was stirred overnight. The pyridine was then removed under reduced pressure and the crude material was subjected to chromatography (gradient: 0% CH₃OH—CH₂Cl₂ to 10%) to yield 5 (1.4 g, 42%) as a yellow oil.

### Step 6. Preparation of Racemic (cis) Lithium 10-(3-((bis(4-methoxyphenyl)-(phenyl)methoxy)methyl)-4-(hydroxymethyl)-3,4-dimethylpyrrolidin-1-yl)-10-oxodecanoate 306

To a solution of compound 305 (3.0 g, 4.6 mmol) in THF (50 mL) and water (50 mL) was added lithium hydroxide (121 mg, 5.0 mmol). The solution was stirred for 4 hours at room temperature then concentrated to remove the THF. The remaining aqueous solution was freeze dried overnight to afford a pale pink solid (2.9 g, quantitative). Compound 306 was prepared as a mixture of two cis-diastereomers.

### Scheme 57 Synthesis of Peracetylated Galactosamine 507

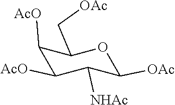 [SMILES: CC(=O)NC1C(OC(C)=O)OC(COC(C)=O)C(OC(C)=O)C1OC(C)=O]

Galactosamine hydrochloride (250 g, 1.16 mol) in pyridine (1.5 L) is treated with acetic anhydride (1.25 L, 13.2 mol) over 45 minutes. After stirring overnight the reaction mixture is divided into three 1 L portions. Each 1 L portion is poured into 3 L of ice water and mixed for one hour. After mixing the solids are filtered off, combined, frozen over liquid nitrogen and then lyophilized for five days to yield peracetylated galactosamine 507 (369.4 g, 82%) as a white solid. Rf (0.58, 10% MeOH—CH₂Cl₂).

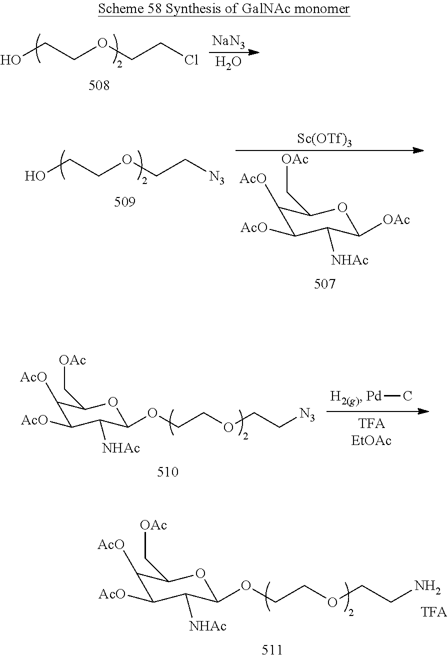

### Step 1 Preparation of Compound 509

A solution of 2-[2-(2-chloroethoxy)]ethanol 508 (100 g, 593 mmol) in water (1 L) is treated with NaN₃ (77 g, 1.19 mol) and heated (90° C.). After stirring (72 hours) the solution is cooled (RT) and extracted (4×) with CH₂Cl₂. The combined organics are washed with brine, dried (MgSO4), filtered, concentrated and used without further processing. Compound 509 (88.9 g, 86%) is obtained as a pale yellow oil.

### Step 2 Preparation of Compound 510

A solution of 507 (2.76 g, 7.1 mmol) and 509 (1.37 g, 7.8 mmol) in 1,2-dichloroethane (40 mL) is treated with Sc(OTf)₃ (174 mg, 0.36 mmol) and heated (85° C.). After stirring (2 hours) the mixture is cooled (RT) and quenched by the addition of TEA (4 mL) and concentrated. The crude material is subjected to chromatography to yield 510 (3.03 g, 85%) as a pale yellow foam.

### Step 3 Preparation of Compound 511

A solution of 510 (3.02 g, 5.99 mmol) and Pd/C (300 mg, 10% Pd loading—wet support) in EtOAc (30 mL) is treated with TFA (576 μL, 7.5 mmol). The reaction mixture is purged with hydrogen gas (45 min) then purged with nitrogen gas (10 min), then filtered through celite. The filtrate is concentrated and then subjected to chromatography to yield 511 (2.67 g, 75%) as a brown foam.

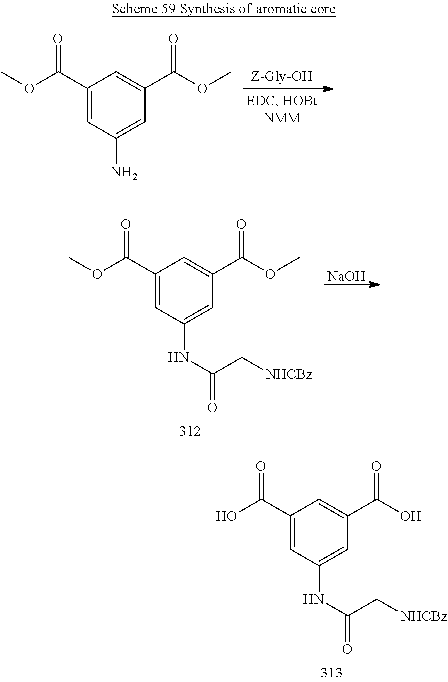

### Step 1. Preparation of Dimethyl 5-(2-((2-oxo-2-phenyl-1λ²-ethyl)amino)acetamido)-isophthalate 312

A solution of dimethyl 5-aminoisophthalate (5 g, 24 mmol), Z-Gly-OH (5 g, 24 mmol), EDC (5 g, 26.3 mmol), HOBt (3.6 g, 26.3 mmol), NMM (2.9 mL, 26.3 mmol) in DMF (50 mL) was stirred overnight at room temperature. Upon completion, the reaction mixture was diluted with ethyl acetate (250 mL) and washed with each 1M HCl (2×100 mL), saturated sodium bicarbonate (1×100 mL) and brine (2×100 mL). Dry on magnesium sulfate, filter and concentrate to dryness to afford Dimethyl 5-(2-((2-oxo-2-phenyl-1λ²-ethyl)amino)-acetamido)isophthalate as a colorless solid (7.2 g, 79%).

### Step 2. Preparation of 5-(2-((2-oxo-2-phenyl-1λ²-ethyl)amino)acetamido)isophthalic acid 313

To a solution of methyl 5-(2-((2-oxo-2-phenyl-1λ²-ethyl)amino)acetamido)isophthalate (7.2 g) in methanol (25 mL) and THF (25 mL) was added 1M NaOH (25 mL). The solution was stirred at room temperature for 2 hours then concentrated to remove THF and MeOH. The aqueous solution remaining was diluted with water (75 mL), cooled on an ice water bath and acidified to pH=1 with 6M HCl. The solid was filtered and washed with water (3×100 mL). The solid was freeze dried to afford 5-(2-((2-oxo-2-phenyl-1λ²-ethyl)amino)acetamido)-isophthalic acid (6.9 g, quantitative).

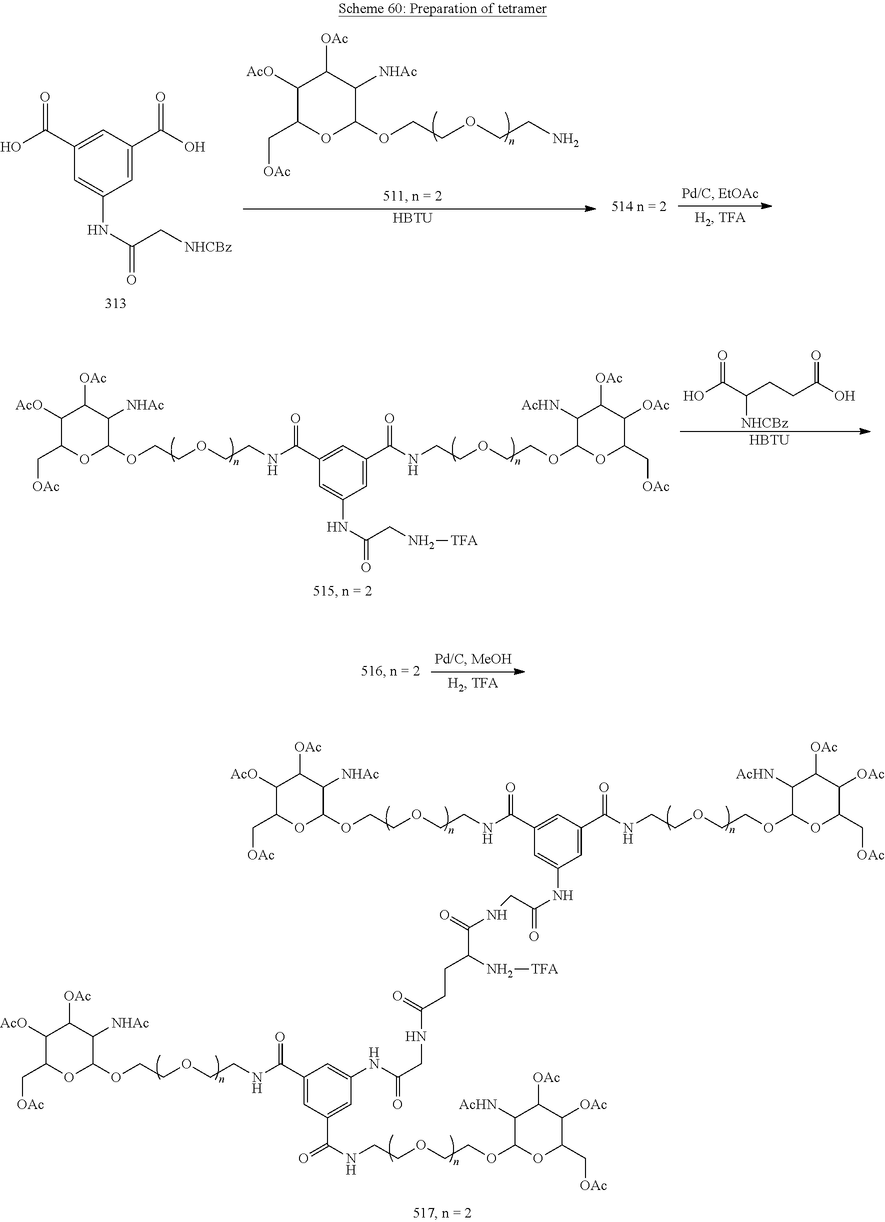

### Step 1 Preparation of Compound 514

A solution of 313 (2.09 g, 5.6 mmol) and 511 (8.34 g, 14.07 mmol) in CH₂Cl₂ (150 mL) is treated with HBTU (6.4 g, 16.9 mmol) and Hunig's base (7.35 mL, 42.2 mmol). After stirring (overnight) the reaction mixture is poured into NaHCO₃ (sat. aq.) then washed with water and brine, dried (MgSO₄), filtered and concentrated. The crude material is subjected to chromatography (gradient 1-12% CH₃OH—CH₂Cl₂) to yield 6 (3.97 g, 55%) as a pale yellow foam.

### Step 2 Preparation of Compound 515

Compound 514 (3.92 g, 3.07 mmol), Pd/C (400 mg, 10% loading—wet support) and trifluoroacetic acid (308 μL, 4 mmol) is purged with H₂. After stirring under H₂ (overnight), the mixture is purged with N₂ (15-20 min) then filtered through celite and concentrated. The crude material is subjected to chromatography to yield 7 (3.36 g, 86%) as a white to cream colored foam.

### Step 3 Preparation of Compound 516

Compound 516 is prepared in the same fashion as 514, from Z-glutamic acid (306 mg, 1.09 mmol) and 515 (3.3 g, 2.6 mmol). Yield 1.66 g, 60%.

### Step 4 Preparation of Compound 517

Compound 517 is prepared in the same fashion as 515. Yield 1.65 g, Quant.

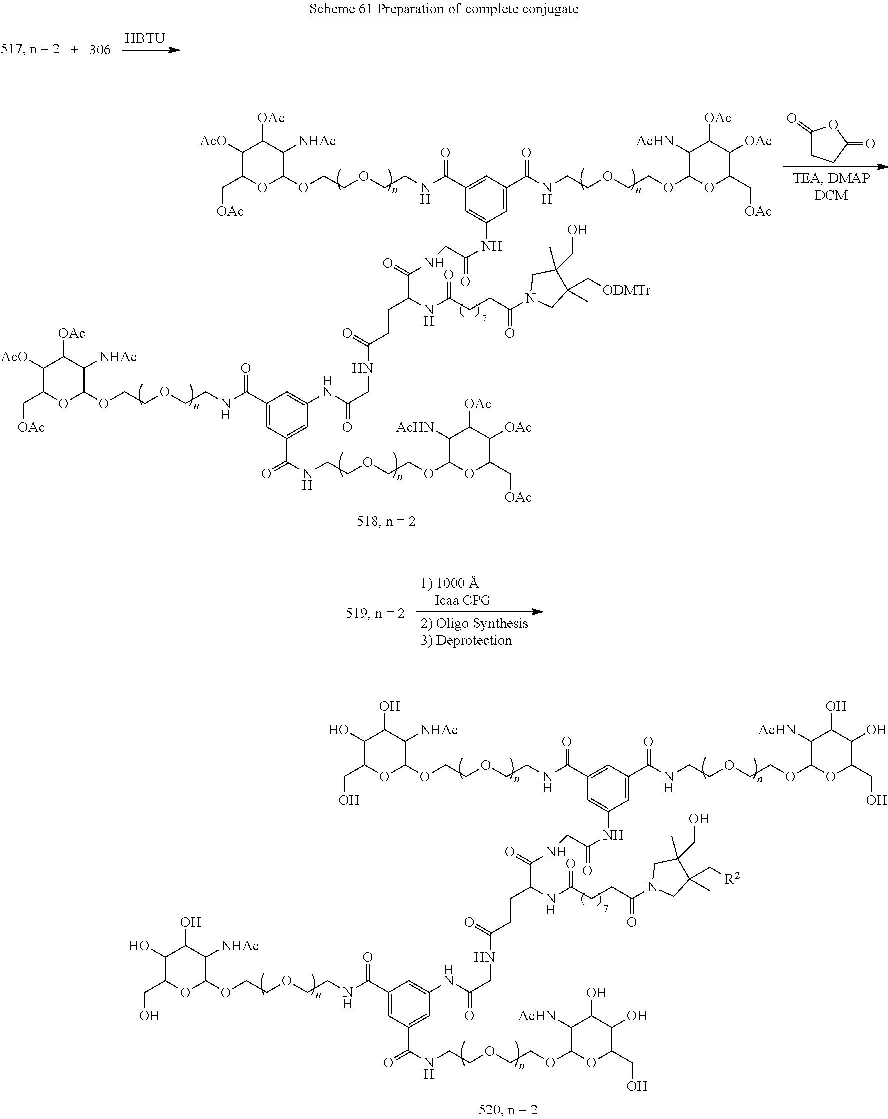

### Step 1 Preparation of Compound 518

A solution of 517 (1.91 g, 0.75 mmol) in CH₂Cl₂ (100 mL) is treated first with Hunig's base (392 μL, 2.25 mmol) then 306 (a mixture of two cis-diastereomers, 509 mg, 0.79 mmol) followed by HBTU (356 mg, 0.94 mmol). After stirring (overnight) the solution was poured into NaHCO₃ (sat. aq.) then washed with water and brine, dried (MgSO4), filtered and concentrated. The crude material is subjected to chromatography to yield 518 (1.19 g, 52%) as a white foam.

### Step 2 Preparation of Compound 519

A solution of 518 (1.19 g, 0.39 mmol) in 1,2 dichloroethane (100 mL) is treated with TEA (542 μL, 3.9 mmol), DMAP (238 mg, 1.95 mmol) and succinic anhydride (195 mg, 1.95 mmol) and heated (85° C.). After stirring (2.5 hours) the solution is removed from heat and treated with CH₃OH (10 mL) and allowed to stir (1 hour). After stirring the mixture is poured into NaHCO₃ (sat. aq.) then washed with brine, dried (MgSO4), filtered and concentrated. The residue obtained is used without further processing. Yield=1.4 g, Quant.

### Step 3 Preparation of conjugate 520

The succinate 519 is loaded onto 1000 Å LCAA (long chain aminoalkyl) CPG (control pore glass) using standard amide coupling chemistry. A solution of diisopropylcarbodiimide (52.6 μmol), N-hydroxy succinimide (0.3 mg, 2.6 μmol) and pyridine (10 μL) in anhydrous acetonitrile (0.3 mL) is added to 519 (20.6 mg, 8 μmol) in anhydrous dichloromethane (0.2 mL). This mixture is added to LCAA CPG (183 mg). The suspension was gently mixed overnight at room temperature. Upon disappearance of 519 (HPLC), the reaction mixture is filtered and the CPG is washed with 1 mL of each dichloromethane, acetonitrile, a solution of 5% acetic anhydride/5% N-methylimidazole/5% pyridine in THF, then THF, acetonitrile and dichloromethane. The CPG is then dried overnight under high vacuum. Loading was determined by standard DMTr assay by UV/Vis (504 nm) to be 19 μmol/g. The resulting GalNAc loaded CPG solid support is employed in automated oligonucleotide synthesis using standard procedures. Nucleotide deprotection followed by removal from the solid support (with concurrent galactosamine acetate deprotection) affords the GalNAc-oligonucleotide conjugate 520.

### Example 27 Synthesis of Targeted Nucleic Acid Conjugates

The following Schemes 101-122 illustrate the preparation of intermediate compounds that can be used to prepare conjugates of formula I. The intermediate compounds and the synthetic processes illustrated in Schemes 1-22 are embodiments of the present invention.

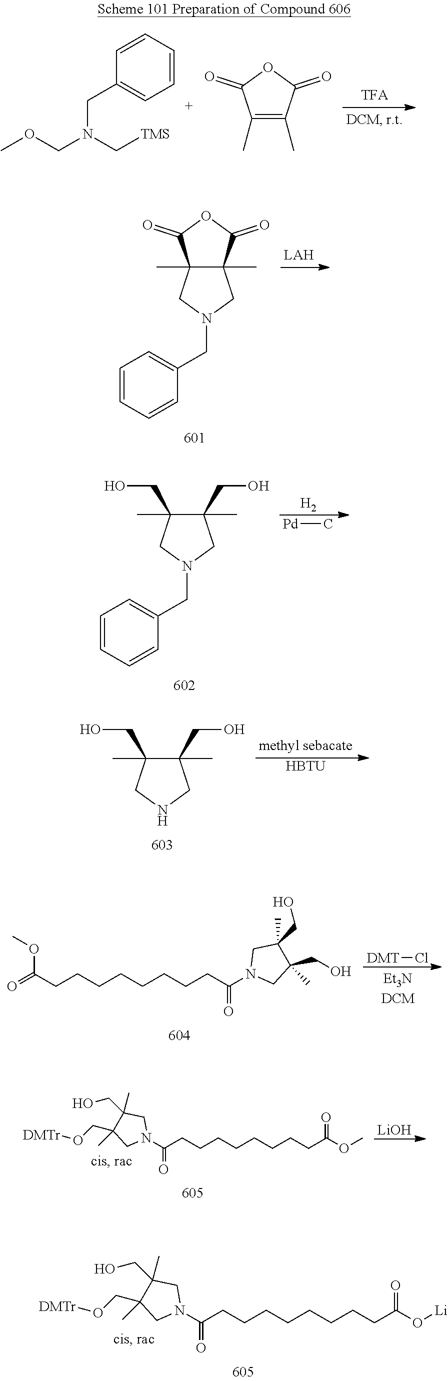

### Step 1. Preparation of (3aR,6aS)-5-Benzyl-3a,6a-dimethyltetrahydro-1H-furo[3,4-c]pyrrole-1,3(3aH)-dione 601

To a cooled solution (0° C.) of 3,4-dimethylfuran-2,5-dione (40 g, 317 mmol) and N-benzyl-1-methoxy-N-((trimethylsilyl)methyl)methanamine (94.1 g, 396.5 mmol) in DCM (600 ml) was slowly added trifluoroacetic acid (732 μl). Stir overnight allowing the solution to slowly warm to RT. The reaction mixture was concentrated to dryness, dissolved in EtOAc (500 ml), washed with saturated sodium bicarbonate (2×500 ml), dried on magnesium sulfate, filtered and concentrated to dryness. Purification by column chromatography on silica gel (gradient: 20% ethyl acetate in hexanes to 100% ethyl acetate) afforded (3aR,6aS)-5-Benzyl-3a,6a-dimethyltetrahydro-1H-furo[3,4-c]pyrrole-1,3(3aH)-dione as a yellow oil (53.7 g, 65%). Rf 0.85 40% EtOAc-Hexane

### Step 2. Preparation of ((3R,4S)-1-Benzyl-3,4-dimethylpyrrolidine-3,4-diyl)dimethanol 602

To a cooled (0° C.) solution of (3aR,6aS)-5-Benzyl-3a,6a-dimethyltetrahydro-1H-furo[3,4-c]pyrrole-1,3(3aH)-dione (53.7 g, 205.7 mmol) in anhydrous diethyl ether (750 ml) was added slowly lithium aluminum hydride pellets (17.6 g, 463 mmol) in portions over an afternoon. The solution was stirred overnight warming to room temperature as the ice water bath melted. Upon completion, the reaction was cooled to 0° C. and very slowly quenched with 25 ml of 5M NaOH followed by 12 ml of water. Stir for 30 minutes then add magnesium sulfate and filter. The filtrate was concentrated to afford ((3R,4S)-1-Benzyl-3,4-dimethylpyrrolidine-3,4-diyl)dimethanol as a colorless oil (33.6 g, 65%). Rf 0.25 10% CH₃OH—CH₂Cl₂

### Step 3. Preparation of ((3R,4S)-3,4-Dimethylpyrrolidine-3,4-diyl)dimethanol 603

To a solution of ((3R,4S)-1-Benzyl-3,4-dimethylpyrrolidine-3,4-diyl)dimethanol (40.1 g, 161 mmol) in methanol (300 ml) was added 10% palladium on activated charcoal wet (4 g). The solution was stirred vigorously under a hydrogen atmosphere for 16 hours. Upon completion the solution was filtered through Celite, and concentrated to dryness to afford ((3R,4S)-3,4-Dimethylpyrrolidine-3,4-diyl)dimethanol as a colorless solid (24 g, 94%). Rf 0.05 10% CH₃OH—CH₂Cl₂

### Step 4. Preparation of Methyl 10-((3R,4S)-3,4-bis(hydroxymethyl)-3,4-dimethylpyrrolidin-1-yl)-10-oxodecanoate 604

A solution of 3 (24 g, 151 mmol) and monomethyl sebacate (34.2 g, 159 mmol) in CH₂Cl₂ (11) was treated with HBTU (62.9 g, 166 mmol) and Hunig's base (105 ml, 604 mmol). After stirring overnight the mixture was washed with NaHCO₃ (sat. aq.), water and brine, then dried (MgSO4), filtered and concentrated. The crude material was subjected to chromatography (gradient: 0% CH₃OH—CH₂Cl₂ to 20%) to yield 604 (41.5 g, 77%). Rf 0.55 10% CH₃OH—CH₂Cl₂

### Step 5. Preparation of methyl 10-(3-((bis(4-methoxyphenyl)(phenyl)methoxy)methyl)-4-(hydroxymethyl)-3,4-dimethylpyrrolidin-1-yl)-10-oxodecanoate 605

A solution of 604 (41.5 g, 116 mmol) and 4,4′-Dimethoxytrityl chloride (38.8 g, 116 mmol) in pyridine (400 ml) was stirred overnight. The pyridine was then removed under reduced pressure and the crude material was subjected to chromatography (gradient: 0% CH₃OH—CH₂Cl₂ to 10%) to yield 605 (29.5 g, 39%) as a yellow oil. Rf 0.5 5% CH₃OH—CH₂Cl₂

### Step 6. Preparation of lithium 10-(3-((bis(4-methoxyphenyl)(phenyl)methoxy)methyl)-4-(hydroxymethyl)-3,4-dimethylpyrrolidin-1-yl)-10-oxodecanoate 606

To a solution of compound 605 (29.5 g, 45 mmol) in THF (250 ml) and water (250 ml) was added lithium hydroxide (1.19 g, 50 mmol). The solution was stirred for 18 hours at room temperature then concentrated to remove the THF. The remaining aqueous solution was freeze dried overnight to afford 606 as a pale purple solid (28.5 g, 98%). Rf 0.56 10% CH₃OH—CH₂Cl₂

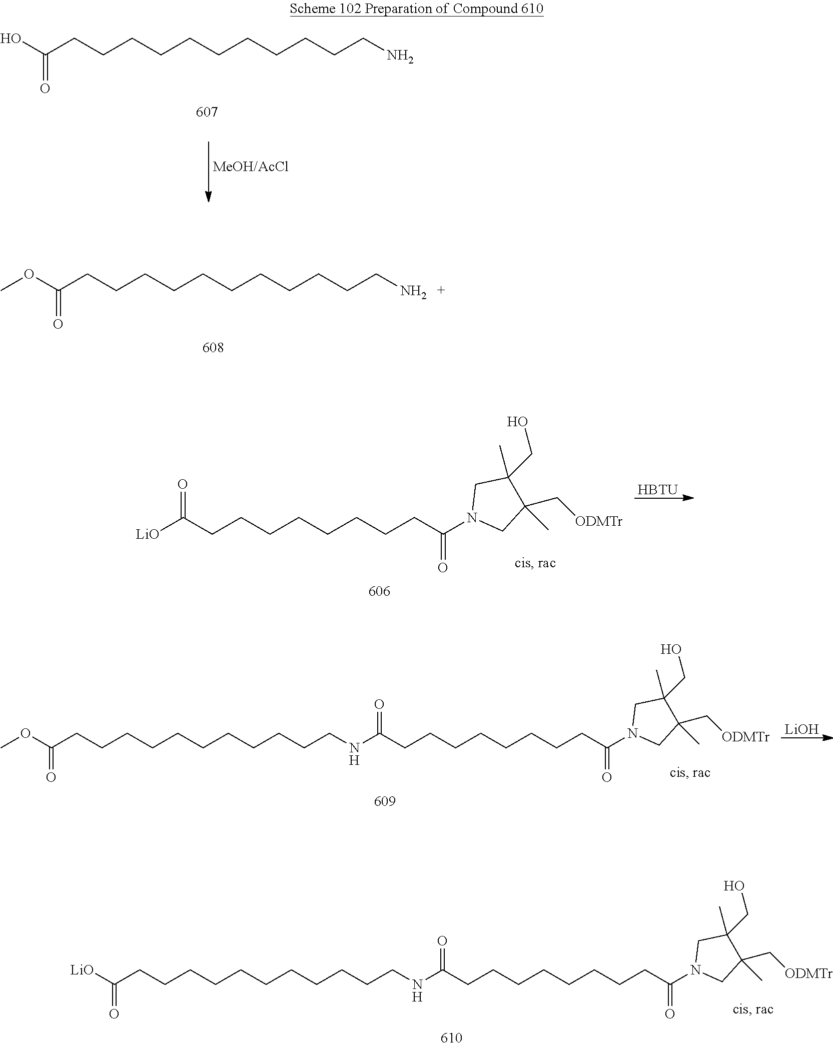

### Step. 1. Preparation of methyl 12-aminododecanoate 608

12-Aminoundecanoic acid 607 (10 g, 4.64 mmol) was stirred in MeOH at RT. Acetyl chloride (856 μl, 12 mmol) was added dropwise and the reaction stirred for 1.5 hr. The solvent was removed in-vacuo, the residue taken up in MTBE and chilled in the fridge overnight. The resultant precipitate was collected by filtration, washed with ice cold MTBE and dried under high vacuum to afford methyl 12-aminododecanoate 608.

### Step 2. Preparation of methyl 12-(10-(3-((bis(4-methoxyphenyl)(phenyl)methoxy)methyl)-4-(hydroxymethyl)-3,4-dimethylpyrrolidin-1-yl)-10-oxodecanamido)dodecanoate 609

Lithium 10-(3-((bis(4-methoxyphenyl)(phenyl)methoxy)methyl)-4-(hydroxymethyl)-3,4-dimethylpyrrolidin-1-yl)-10-oxodecanoate (606) (2 g, 3.1 mmol), methyl 12-aminododecanoate (608) (778 mg, 3.1 mmol), HBTU (1.2 g, 3.1 mmol) and TEA (1.4 ml, 10 mmol) were stirred in DCM at RT O/N. The precipitate was removed by filtration, the filtrate concentrated in-vacuo and the residue purified by column chromatography (5% MeOH, DCM). TLC showed two close running spots with identical mass that were assigned as geometric isomers and pooled together to methyl 12-(10-(3-((bis(4-methoxyphenyl)(phenyl)methoxy)methyl)-4-(hydroxymethyl)-3,4-dimethylpyrrolidin-1-yl)-10-oxodecanamido)dodecanoate (609) in quantitative fashion.

### Step 3. Preparation of lithium 12-(10-(3-((bis(4-methoxyphenyl)(phenyl)methoxy)-methyl)-4-(hydroxymethyl)-3,4-dimethylpyrrolidin-1-yl)-10-oxodecanamido)-dodecanoate 610

Methyl 12-(10-(3-((bis(4-methoxyphenyl)(phenyl)methoxy)methyl)-4-(hydroxymethyl)-3,4-dimethylpyrrolidin-1-yl)-10-oxodecanamido)dodecanoate 609 (3.1 mmol) was stirred in THF:H₂O (50:50) with LiOH (88 mg, 3.7 mmol) at RT O/N. Reaction was confirmed by TLC and the THF removed in-vacuo. The aqueous solution was frozen in liquid N₂ and lyophilized for 48 hours to give lithium 12-(10-(3-((bis(4-methoxyphenyl)(phenyl)methoxy)methyl)-4-(hydroxymethyl)-3,4-dimethylpyrrolidin-1-yl)-10-oxodecanamido)dodecanoate 610 quantitatively.

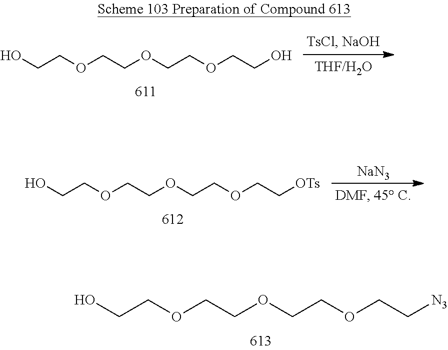 [SMILES: OCCOCCOCCOCCO ; Cc1ccc(S(=O)(=O)OCCOCCOCCOCCO)cc1 ; [N-]=[N+]=NCCOCCOCCOCCO ; "NaN3"]

### Step 1. Preparation of 2-(2-(2-(2-hydroxyethoxy)ethoxy)ethoxy)ethyl 4-methylbenzenesulfonate 612

A solution of tetraethylene glycol (611) (934 g, 4.8 mol) in THF (175 ml) and aqueous NaOH (5M, 145 ml) was cooled (0° C.) and treated with p-Toluensulfonyl chloride (91.4 g, 480 mmol) dissolved in THF (605 ml) and then stirred for two hours (0° C.). The reaction mixture was diluted with water (3 L) and extracted (3×500 ml) with CH₂Cl₂. The combined extracts were washed with water and brine then dried (MgSO₄), filtered and concentrated to afford 2-(2-(2-(2-hydroxyethoxy)ethoxy)ethoxy)ethyl 4-methylbenzenesulfonate (612) (140 g, 84%) as a pale yellow oil. Rr (0.57, 10% MeOH—CH₂Cl₂).

### Step 2. Preparation of 2-(2-(2-(2-azidoethoxy)ethoxy)ethoxy)ethan-1-ol 613

A solution of 612 (140 g, 403 mmol) in DMF (880 ml) was treated with sodium azide (131 g, 2.02 mol) and heated (45° C.) overnight. A majority of the DMF was removed under reduced pressure and the residue was dissolved in CH₂Cl₂ (500 ml) and washed (3×500 ml) with brine then dried (MgSO₄), filtered and concentrated. The residue was passed through a short bed of silica (5% MeOH—CH₂Cl₂) and concentrated to yield 2-(2-(2-(2-azidoethoxy)ethoxy)ethoxy)ethan-1-ol 613 (65 g, 74%) as a yellow oil. Rr (0.56, 10% MeOH—CH₂Cl₂).

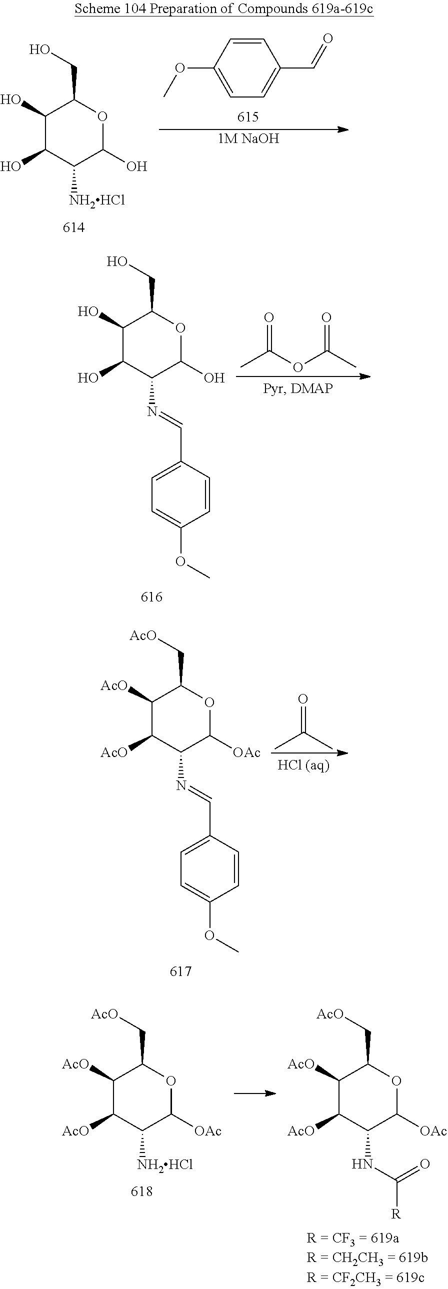

### Step 1. Preparation of (3R,4R,5R,6R)-6-(hydroxymethyl)-3-(((E)-4-methoxybenzylidene) amino)tetrahydro-2H-pyran-2,4,5-triol 616 D-Galactosamine HCl (614) (9 g, 41.7 mmol) was stirred in 1 M NaOH solution at RT

Anisaldehyde (51 ml, 420 mmol) was added and the reaction stirred vigorously until solidification. The solid reaction was kept at 4° C. for 16 h. Ice cold water (200 ml) was added and the resultant solid collected by filtration, washing with ice cold EtOH/Et₂O (1:1). The solid was dried to a constant weight to give (3R,4R,5R,6R)-6-(hydroxymethyl)-3-(((E)-4-methoxybenzylidene) amino)tetrahydro-2H-pyran-2,4,5-triol (616) (9.81 g, 78%).

### Step 2. Preparation of (3R,4R,5R,6R)-6-(acetoxymethyl)-3-(((E)-4-methoxybenzylidene)amino) tetrahydro-2H-pyran-2,4,5-triyl triacetate 6 17

(3R,4R,5R,6R)-6-(Hydroxymethyl)-3-(((E)-4-methoxybenzylidene)amino)tetrahydro-2H-pyran-2,4,5-triol (616) (9.81 g, 30 mmol) was stirred in pyridine at 0° C. Acetic anhydride (34 ml) followed by DMAP (100 mg, cat) was added and the reaction stirred for 16 h allowing to warm to RT slowly. The resultant solution was poured onto crushed ice and kept at 4° C. for 16 h. The reaction was extracted with EtOAc (×3) and the combined organics washed with H₂O and brine, dried (Na₂SO₄) and concentrated in-vacuo to give (3R,4R,5R,6R)-6-(acetoxymethyl)-3-(((E)-4-methoxybenzylidene)amino)tetrahydro-2H-pyran-2,4,5-triyl triacetate (617) (6.0 g, 43%).

### Step 3. Preparation of (3R,4R,5R,6R)-6-(acetoxymethyl)-3-aminotetrahydro-2H-pyran-2,4,5-triyl triacetate hydrochloride 618

(3R,4R,5R,6R)-6-(Acetoxymethyl)-3-(((E)-4-methoxybenzylidene)amino)tetrahydro-2H-pyran-2,4,5-triyl triacetate (617) (6.0 g, 43%) was heated at reflux in acetone (300 ml). HCl (aq) (5N, 3.0 ml) was added and the reaction stirred for 15 mins. After cooling, Et₂O (400 ml) was added and the reaction kept at 4° C. for 16 h. The resultant solid was collected by filtration, washing twice with ice cold Et₂O. The solid was dried to a constant weight to give (3R,4R,5R,6R)-6-(acetoxymethyl)-3-aminotetrahydro-2H-pyran-2,4,5-triyl triacetate hydrochloride (618) (4.17 g, 84.4%).

### Step 4a. Preparation of (3R,4R,5R,6R)-6-(acetoxymethyl)-3-(2,2,2-trifluoroacetamido) tetrahydro-2H-pyran-2,4,5-triyl triacetate 619a

(3R,4R,5R,6R)-6-(Acetoxymethyl)-3-aminotetrahydro-2H-pyran-2,4,5-triyl triacetate hydrochloride (618) (13.5 g, 35.2 mmol) and TEA (7.83 g, 77.4 mmol) were stirred in DCM at RT. TFAA (8.13 g, 38.7 mmol) in DCM was added dropwise and the reaction stirred for 1h. The reaction was diluted with DCM, washed sequentially with 1M HCl, saturated NaHCO₃, water and brine, dried (Na₂SO₄) and concentrated in-vacuo. The residue was purified by automated flash chromatography (5% MeOH/DCM) to give (3R,4R,5R,6R)-6-(acetoxymethyl)-3-(2,2,2-trifluoroacetamido)tetrahydro-2H-pyran-2,4,5-triyl triacetate (619a) (9.64 g, 61.8%). Product confirmed by MS (ESI+ve).

### Step 4b. Preparation of (3R,4R,5R,6R)-6-(acetoxymethyl)-3-propionamidotetrahydro-2H-pyran-2,4,5-triyl triacetate 619b

This compound was prepared in an analogous fashion to (3R,4R,5R,6R)-6-(acetoxymethyl)-3-(2,2,2-trifluoroacetamido)tetrahydro-2H-pyran-2,4,5-triyl triacetate (619a) using propionic anhydride instead of TFAA to give (3R,4R,5R,6R)-6-(acetoxymethyl)-3-propionamidotetrahydro-2H-pyran-2,4,5-triyl triacetate (619b) (1.2 g, 85.3%). Product confirmed by MS (ESI+ve).

### Step 4c. Preparation of (3R,4R,5R,6R)-6-(acetoxymethyl)-3-(2,2-difluoropropanamido) tetrahydro-2H-pyran-2,4,5-triyl triacetate 619c

(3R,4R,5R,6R)-6-(Acetoxymethyl)-3-aminotetrahydro-2H-pyran-2,4,5-triyl triacetate hydrochloride (618) (15.34 g, 39.98 mmol), 2,2-difluoropropionic acid (4.4 g, 39.98 mmol), HATU (24.37 g, 64 mmol) and TEA (12.14 g, 120 mmol) were stirred in DMF at RT for 16 h. The reaction was partitioned between EtOAc and water. The organics were separated, washed sequentially with 1M HCl, saturated NaHCO₃, water and brine, dried (Na₂SO₄) and concentrated in-vacuo. The residue was purified by automated flash chromatography (3% MeOH/DCM) to give (3R,4R,5R,6R)-6-(acetoxymethyl)-3-(2,2-difluoropropanamido)-tetrahydro-2H-pyran-2,4,5-triyl triacetate (619c) (15.8 g, 90%). Product confirmed by MS (ESI+ve).

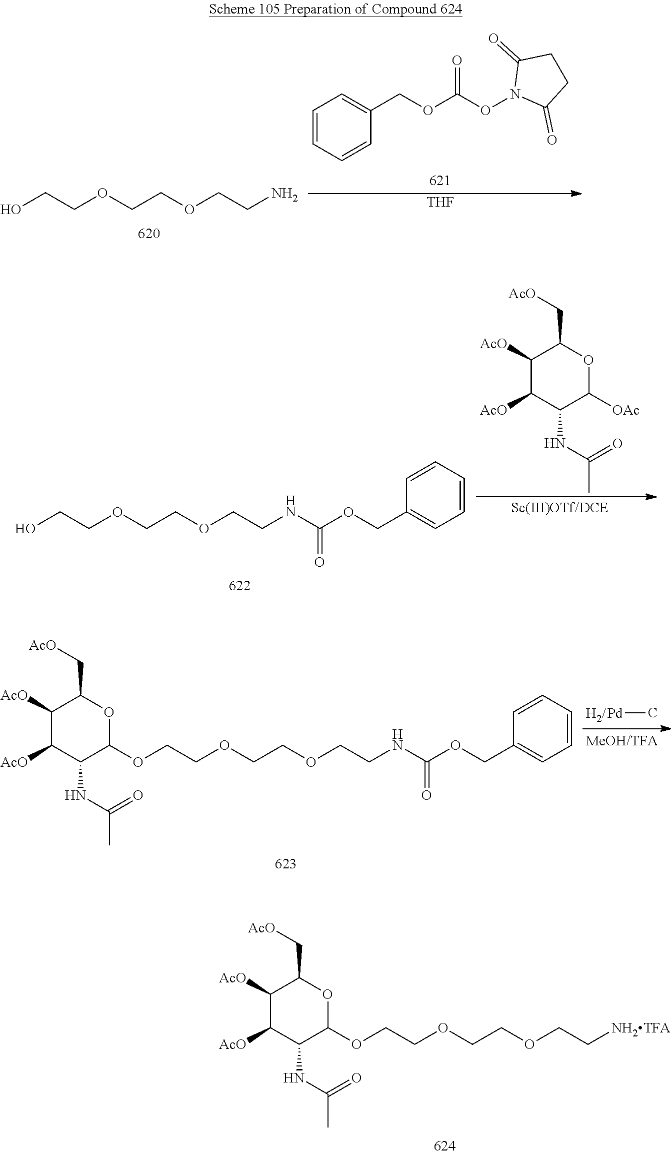

### Step 1. Preparation of benzyl (2-(2-(2-hydroxyethoxy)ethoxy)ethyl)carbamate 622

A solution of the amino alcohol (620) (313.6 g 2.1 mol) in THF (3.5 L) was treated, portion-wise, with N-(Benzyloxycarbonyloxy)succinimide (621) (550 g, 2.21 mol). Once the reaction was complete (18 h) the THF was removed under reduced pressure and the residue dissolved in CH₂Cl₂ (2.5 L), then washed with an equal volume of HCl (1 M), NaHCO₃ (Sat. Aq.), H₂O and brine. The organic extract was dried (MgSO₄), filtered and concentrated. The crude material (600 g) was subjected to chromatography (4 kg silica; 1-12% CH₃OH—CH₂Cl₂) to yield HO-Trig-NHZ (622) (468 g, 78%) as a clear-yellow viscous oil.

### Step 2. Preparation of (2R,3R,4R,5R)-5-acetamido-2-(acetoxymethyl)-6-((3-oxo-1-phenyl-2,7,10-trioxa-4-azadodecan-12-yl)oxy)tetrahydro-2H-pyran-3,4-diyl diacetate 623

A heterogeneous mixture of galactosamine pentaacetate (715.2 g. 1.84 mol) and HO-Trig-NHZ (622) (400 g, 1.41 mol) in 1,2 dichloroethane (10 L) was treated with 5 mol % Sc(OTf)₃ (34.6 g, 70.5 mmol) and heated (85° C.). After stirring (5.5 h) the solution became clear and homogeneous, the reaction was cooled and washed with NaHCO₃(Sat. Aq.), HCl (1M), H₂O and brine. The organic extracts were dried (MgSO₄), filtered and concentrated. The crude material (900 g) was treated with EtOAc (900 ml) which gave a milky heterogeneous mixture that was filtered through a course frit thus removing residual pentaacetate. The filtrate was concentrated, and the crude material was subjected to chromatography (5 kg silica; 0-10% CH₃OH-EtOAc) to yield the glycosylation product (623) (751 g. 87%) as a light brown foam.

### Step 3. Preparation of (2R,3R,4R,5R)-5-acetamido-2-(acetoxymethyl)-6-(2-(2-(2-((2,2,2-trifluoroacetyl)-14-azaneyl)ethoxy)ethoxy)ethoxy)tetrahydro-2H-pyran-3,4-diyl diacetate 6 24

A solution of Gal-trig-NHZ (623) (750 g, 1.22 mol), TFA (103.8 ml, 1.35 mol) and Pd/C (10%—wet support, 75 g) was purged with H₂. After vigorous stirring (4.5h) the reaction mixture was purged with N₂ (30 min) then filtered through Celite and concentrated. The resultant brown foam (712 g, 99%) was used in the next step without further processing.

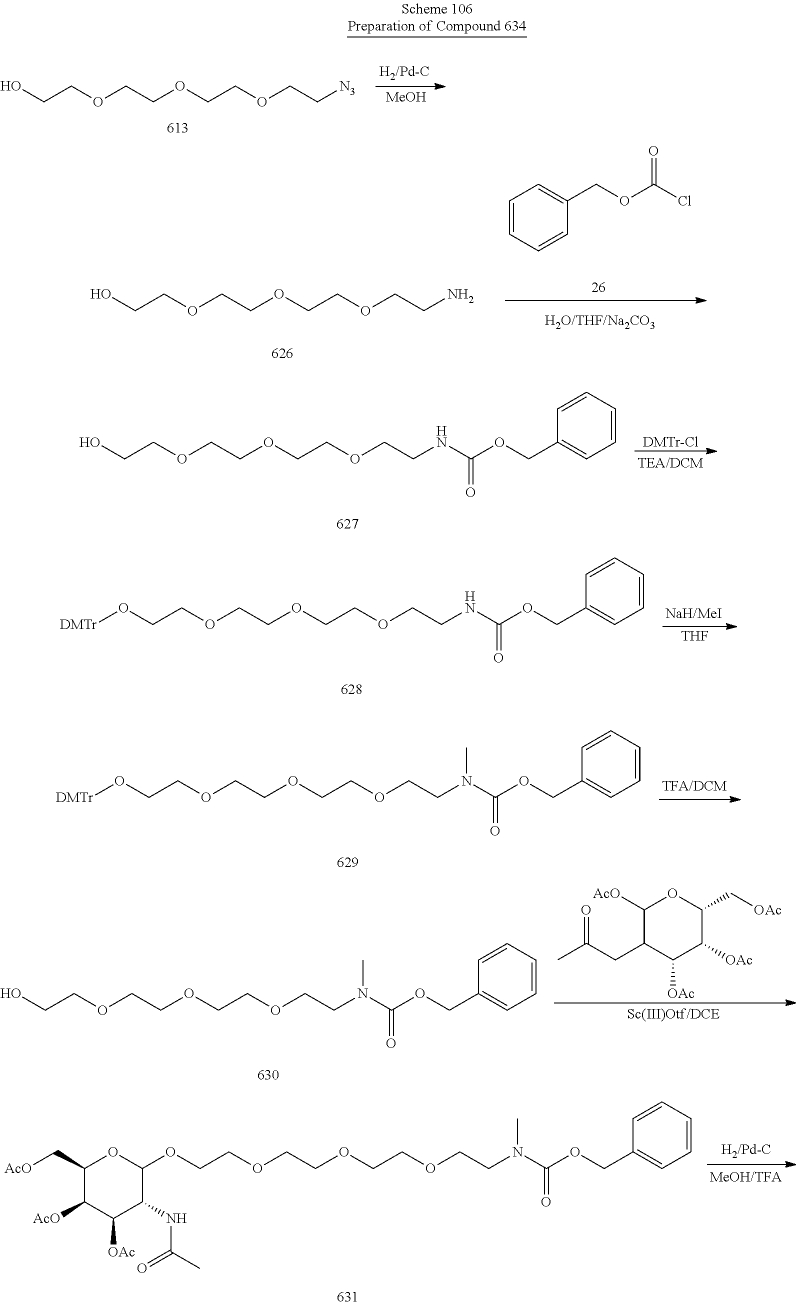

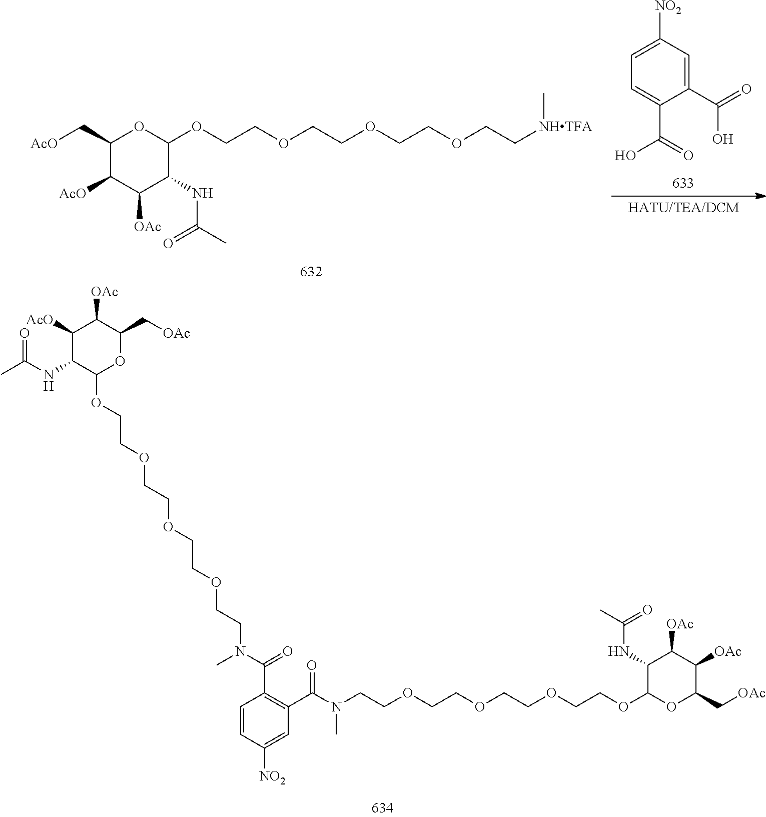

### Step 1. Preparation of 2-(2-(2-(2-aminoethoxy)ethoxy)ethoxy)ethan-1-ol 625

2-(2-(2-(2-Azidoethoxy)ethoxy)ethoxy)ethan-1-ol (613) (70.0 g, 318 mmol) was stirred in MeOH at RT. The reaction was hydrogenated over 10% PD-C (7 g) for 16 h. The reaction was filtered through celite and concentrated in-vacuo to give 2-(2-(2-(2-aminoethoxy)ethoxy)ethoxy)ethan-1-ol (625) (61.4 g, 100%) which was used without further purification. Product confirmed by MS (ESI+ve).

### Step 2. Preparation of benzyl (2-(2-(2-(2-hydroxyethoxy)ethoxy)ethoxy)ethyl)-carbamate 627

2-(2-(2-(2-Aminoethoxy)ethoxy)ethoxy)ethan-1-ol (625) (61.4 g, 318 mmol) was stirred in H₂O (500 ml) with Na₂CO₃ (50.51 g, 476 mmol) at 5° C. Benzyl chloroformate (626) (65.0 g, 381 mmol) in THF (480 ml) was added dropwise and the reaction stirred for 16 h allowing to warm to RT. THF was removed in-vacuo and the aqueous layer extracted with EtOAc (×3). The combined organics were dried (Na₂SO₄), concentrated in-vacuo and the residue purified by automated flash chromatography (5% MeOH/DCM) to give benzyl (2-(2-(2-(2-hydroxyethoxy)ethoxy)ethoxy) ethyl)carbamate (627) (23.6 g, 22.7%). Product confirmed by MS (ESI+ve).

### Step 3. Preparation of benzyl (1,1-bis(4-methoxyphenyl)-1-phenyl-2,5,8,11-tetraoxatridecan-13-yl)carbamate 628

(2-(2-(2-(2-Hydroxyethoxy)ethoxy)ethoxy)ethyl)carbamate (627) (23.6 g, 72.1 mmol) and TEA (7.7 g, 75.7 mmol) were stirred in DCM at RT. DMTr-Cl (25.65 g, 75.7 mmol) was added and the reaction stirred at RT for 2 h. The reaction was washed sequentially with saturated NaHCO₃, water and brine, dried (Na₂SO₄) and concentrated in-vacuo. The residue was purified by automated flash chromatography (50% EtOAc/Hex) to give (1,1-bis(4-methoxyphenyl)-1-phenyl-2,5,8,11-tetraoxatridecan-13-yl)carbamate (v28) (25.5 g, 56.2%). Product confirmed by MS (ESI+ve).

### Step 4. Preparation of benzyl (1,1-bis(4-methoxyphenyl)-1-phenyl-2,5,8,11-tetraoxatridecan-13-yl)(methyl)carbamate 629

(1,1-Bis(4-methoxyphenyl)-1-phenyl-2,5,8,11-tetraoxatridecan-13-yl)carbamate (628) (25.5 g, 40.5 mmol) and MeI (46.0 g, 324 mmol) were stirred in dry THF at 0° C. NaH (60% dispersion in mineral oil) (2.92 g, 121.5 mmol) was added and the reaction stirred at 0° C. then at RT for 1 h. The reaction was partitioned between EtOAc and H₂O. The organics were separated, dried (Na₂SO₄) and concentrated in-vacuo. The residue was purified by automated flash chromatography (50% EtOAc/Hex) to give benzyl (1,1-bis(4-methoxyphenyl)-1-phenyl-2,5,8,11-tetraoxatridecan-13-yl)(methyl)carbamate (629) (26.06 g, 100%). Product confirmed by MS (ESI+ve).

### Step 5. Preparation of benzyl (2-(2-(2-(2-hydroxyethoxy)ethoxy)ethoxy)ethyl)(methyl) carbamate 630

Benzyl (1,1-bis(4-methoxyphenyl)-1-phenyl-2,5,8,11-tetraoxatridecan-13-yl)(methyl)carbamate (629) (26.06 g, 40.5 mmol) was stirred in DCM at RT. TFA (5.1 g, 44.5 mmol) was added and stirred for 1 h. 2 additional equivalents of TFA were added and the reaction stirred for 16 h. The reaction was concentrated in-vacuo and the residue purified by automated flash chromatography (5% MeOH/DCM) to give benzyl (2-(2-(2-(2-hydroxyethoxy)ethoxy)ethoxy)ethyl)(methyl) carbamate (630) (6.76 g, 48.9%). Product confirmed by MS (ESI+ve).

### Step 6. Preparation of (2R,3R,4R,5R)-5-acetamido-2-(acetoxymethyl)-6-((4-methyl-3-oxo-1-phenyl-2,7,10,13-tetraoxa-4-azapentadecan-15-yl)oxy)tetrahydro-2H-pyran-3,4-diyl diacetate 631

(2-(2-(2-(2-Hydroxyethoxy)ethoxy)ethoxy)ethyl)(methyl) carbamate (630) (6.76 g, 19.8 mmol), (3R,4R,5R,6R)-3-acetamido-6-(acetoxymethyl)tetrahydro-2H-pyran-2,4,5-triyl triacetate (7.71 g, 19.8 mmol) and Sc(III)OTf (0.49 g, 1.0 mmol) were heated at reflux in DCE for 2 h. After cooling, the reaction was quenched with TEA and washed sequentially with 1M HCl, saturated NaHCO₃, water and brine, dried (Na₂SO₄) and concentrated in-vacuo. The residue was purified by automated flash chromatography to give (2R,3R,4R,5R)-5-acetamido-2-(acetoxymethyl)-6-((4-methyl-3-oxo-1-phenyl-2,7,10,13-tetraoxa-4-azapentadecan-15-yl)oxy)tetrahydro-2H-pyran-3,4-diyl diacetate (631) (9.37 g, 70.6%). Product confirmed by MS (ESI+ve).

### Step 7. Preparation of (2R,3R,4R,5R)-5-acetamido-2-(acetoxymethyl)-6-((1,1,1-trifluoro-3-methyl-2-oxo-6,9,12-trioxa-314-azatetradecan-14-yl)oxy)tetrahydro-2H-pyran-3,4-diyl diacetate 632

(2R,3R,4R,5R)-5-Acetamido-2-(acetoxymethyl)-6-((4-methyl-3-oxo-1-phenyl-2,7,10,13-tetraoxa-4-azapentadecan-15-yl)oxy)tetrahydro-2H-pyran-3,4-diyl diacetate (631) (9.37 g, 14.0 mmol) and TFA (1.76 g, 15.4 mmol) were stirred in MeOH at RT. The reaction was hydrogenated over 10% Pd—C (1 g) for approx. 2 h. The reaction was filtered through celite and concentrated in-vacuo to give (2R,3R,4R,5R)-5-acetamido-2-(acetoxymethyl)-6-((1,1,1-trifluoro-3-methyl-2-oxo-6,9,12-trioxa-314-azatetradecan-14-yl)oxy)tetrahydro-2H-pyran-3,4-diyl diacetate (632) (9.0 g, 98.9%). The product was used without purification. Product confirmed by MS (ESI+ve).

### Step 8. Preparation of (2R,2′R,3R,3′R,4R,4′R,5R,5′R)-(((4-nitro-1,2-phenylene)bis(2-methyl-1-oxo-5′,8′,11′-trioxa-2′-azatridecane-1,13-diyl))bis(oxy))bis(5-acetamido-2-(acetoxymethyl)tetrahydro-2H-pyran-6,3,4-triyl) tetraacetate 634

(2R,3R,4R,5R)-5-Acetamido-2-(acetoxymethyl)-6-((1,1,1-trifluoro-3-methyl-2-oxo-6,9,12-trioxa-314-azatetradecan-14-yl)oxy)tetrahydro-2H-pyran-3,4-diyl diacetate (32) (4.5 g, 6.93 mmol), 4-nitrophthalic acid (33) (0.73 g, 3.46 mmol), HATU (8.45 g, 22.18 mmol) and TEA (4.21 g, 41.6 mmol) were stirred in DCM at RT for 16 h. The reaction was diluted with DCM and washed sequentially with 1M HCl, saturated NaHCO₃, water and brine, dried (Na₂SO₄) and concentrated in-vacuo. The residue was purified by automated flash column chromatography (10% MeOH/DCM) to give (2R,2′R,3R,3′R,4R,4′R,5R,5′R)-(((4-nitro-1,2-phenylene)bis(2-methyl-1-oxo-5′,8′,11′-trioxa-2′-azatridecane-1,13-diyl))bis(oxy))bis(5-acetamido-2-(acetoxymethyl)tetrahydro-2H-pyran-6,3,4-triyl) tetraacetate (634) (5.0 g, 57.4%). Product confirmed by MS (ESI+ve).

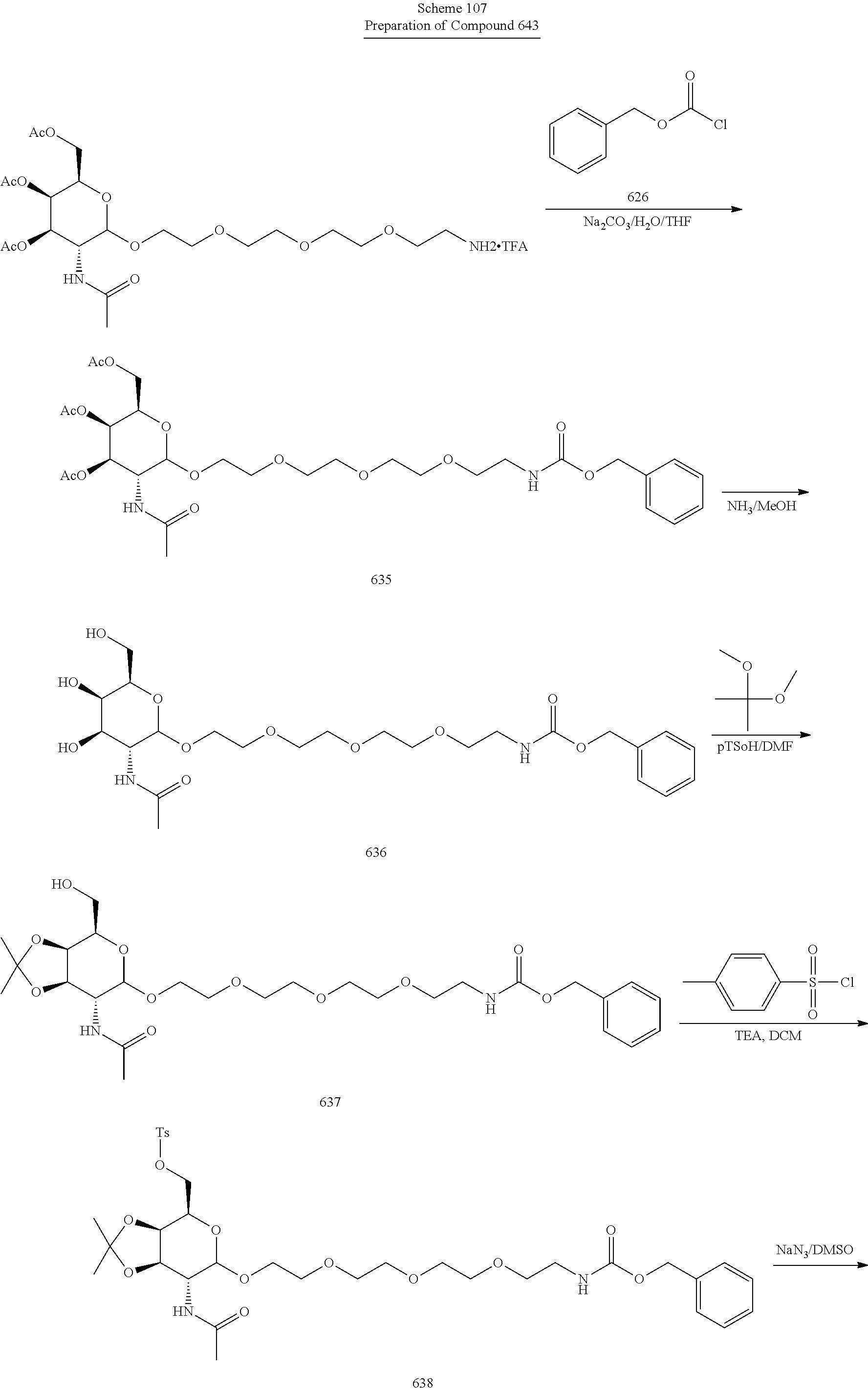

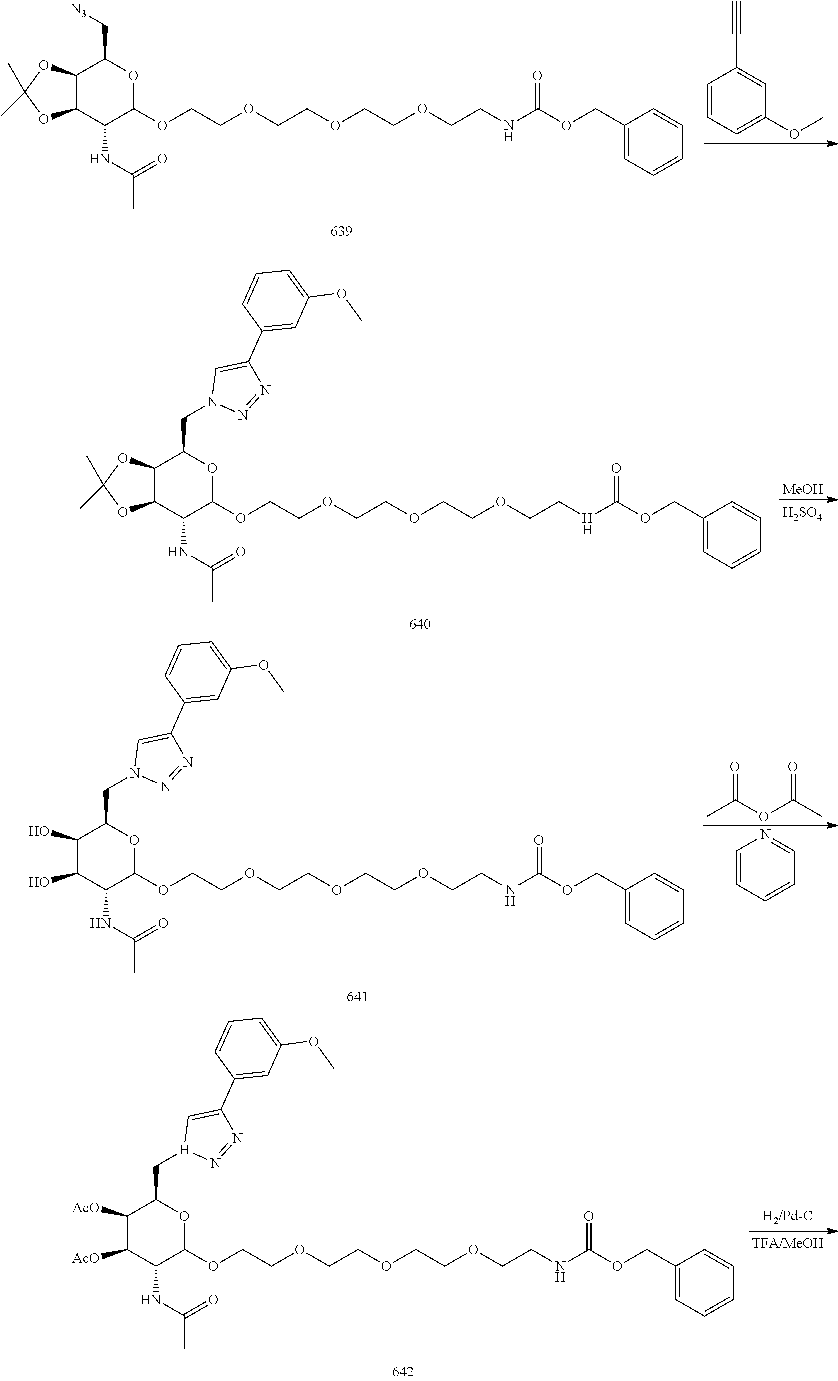

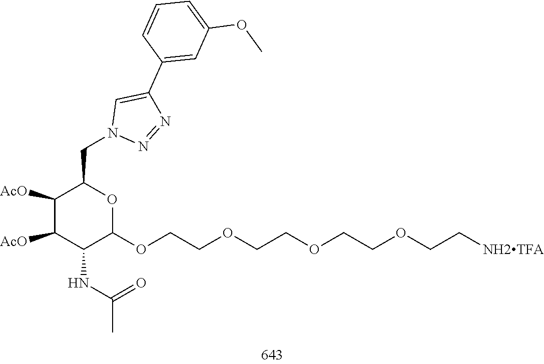

### Step 1. Preparation of (2R,3R,4R,5R)-5-acetamido-2-(acetoxymethyl)-6-((3-oxo-1-phenyl-2,7,10,13-tetraoxa-4-azapentadecan-15-yl)oxy)tetrahydro-2H-pyran-3,4-diyl diacetate

(2R,3R,4R,5R)-5-Acetamido-2-(acetoxymethyl)-6-((1,1,1-trifluoro-2-oxo-6,9,12-trioxa-314-azatetradecan-14-yl)oxy)tetrahydro-2H-pyran-3,4-diyl diacetate (45.0 g, 70.8 mmol) and Na₂CO3 (11.3 g, 106 mmol) were stirred in THF/H₂O (50:50) at RT. Benzyl chloroformate (626) (14.5 g, 85 mmol) was added dropwise and the reaction stirred for 16 h. THF was removed in-vacuo and the aqueous extracted with EtOAc (×3). The organics were washed sequentially with 1M HCl, saturated NaHCO₃, water and brine, dried (Na₂SO₄) and concentrated in-vacuo. The residue was purified by automated flash chromatography (5% MeOH/DCM) to give (2R,3R,4R,5R)-5-acetamido-2-(acetoxymethyl)-6-((3-oxo-1-phenyl-2,7,10,13-tetraoxa-4-azapentadecan-15-yl)oxy)tetrahydro-2H-pyran-3,4-diyl diacetate (635) (25.12 g, 54%). Product confirmed by MS (ESI+ve).

### Step 2. Preparation of benzyl (2-(2-(2-(2-(((3R,4R,5R,6R)-3-acetamido-4,5-dihydroxy-6-(hydroxymethyl)tetrahydro-2H-pyran-2-yl)oxy)ethoxy)ethoxy)ethoxy)ethyl)carbamate

(2R,3R,4R,5R)-5-Acetamido-2-(acetoxymethyl)-6-((3-oxo-1-phenyl-2,7,10,13-tetraoxa-4-azapentadecan-15-yl)oxy)tetrahydro-2H-pyran-3,4-diyl diacetate (635) (25.12 g, 38.3 mmol) was stirred in 7N ammonia solution in MeOH in an airtight sealed reaction vessel at RT for 16 h. The reaction was allowed to evaporate at 50° C. to remove ammonia and the remainder concentrated in-vacuo to give benzyl (2-(2-(2-(2-(((3R,4R,5R,6R)-3-acetamido-4,5-dihydroxy-6-(hydroxymethyl)tetrahydro-2H-pyran-2-yl)oxy)ethoxy)ethoxy)ethoxy)ethyl)carbamate (636) (20.3 g, 100%) which was used in subsequent reactions without further purification. Product confirmed by MS (ESI+ve).

### Step 3. Preparation of benzyl (2-(2-(2-(2-(((3aR,4R,7R,7aR)-7-acetamido-4-(hydroxymethyl)-2,2-dimethyltetrahydro-4H-[1,3]dioxolo[4,5-c]pyran-6-yl)oxy)-ethoxy)ethoxy)ethoxy)ethyl) carbamate 637

Benzyl (2-(2-(2-(2-(((3R,4R,5R,6R)-3-acetamido-4,5-dihydroxy-6-(hydroxymethyl)tetrahydro-2H-pyran-2-yl)oxy)ethoxy)ethoxy)ethoxy)ethyl)carbamate (636) (20.3 g, 38.3 mmol) was stirred in DMF (200 ml) at RT. 2,2-Dimethoxy propane (274 g, 1.6 mol) and pTsOH (cat) were added and the reaction heated at 65° C. for 16 h. The reaction was cooled to RT, TEA (20 ml) added and stirred for 30 min. The solvent was removed in-vacuo, the residue taken up in MeOH/H₂O (10:1) and the reaction refluxed for 1 h. The reaction was concentrated in-vacuo (azeotroping with toluene (×2) and the residue purified by automated flash chromatography (10% MeOH/DCM) to give benzyl (2-(2-(2-(2-(((3aR,4R,7R,7aR)-7-acetamido-4-(hydroxyl-methyl)-2,2-dimethyltetrahydro-4H-[1,3]dioxolo[4,5-c]pyran-6-yl)oxy)ethoxy)ethoxy)-ethoxy)ethyl)carbamate (637) (24.9 g, 100%). Product confirmed by MS (ESI+ve).

### Step 4. Preparation of ((3aR,4R,7R,7aR)-7-acetamido-2,2-dimethyl-6-((3-oxo-1-phenyl-2,7,10,13-tetraoxa-4-azapentadecan-15-yl)oxy)tetrahydro-4H-[1,3]dioxolo[4,5-c]pyran-4-yl)methyl 4-methylbenzenesulfonate 638

Benzyl (2-(2-(2-(2-(((3aR,4R,7R,7aR)-7-acetamido-4-(hydroxymethyl)-2,2-dimethyl-tetrahydro-4H-[1,3]dioxolo[4,5-c]pyran-6-yl)oxy)ethoxy)ethoxy)ethoxy)ethyl)carbamate (637) (25.5 g, 44.8 mmol) and TEA (9.97 g, 98.5 mmol) were stirred in DCM at 0° C. p-Toluene-sulfonyl chloride (18.8 g, 98.5 mmol) in DCM was added and the reaction stirred for 16 h allowing to warm to RT. The reaction was diluted with DCM, washed sequentially with 1M HCl, saturated NaHCO₃, water and brine, dried (Na₂SO₄) and concentrated in-vacuo. The residue was purified by automated flash chromatography (5% MeOH/DCM) to give ((3aR,4R,7R,7aR)-7-acetamido-2,2-dimethyl-6-((3-oxo-1-phenyl-2,7,10,13-tetraoxa-4-azapentadecan-15-yl)oxy)tetrahydro-4H-[1,3]dioxolo[4,5-c]pyran-4-yl)methyl 4-methylbenzenesulfonate (638) (25.5 g, 78.8%). Product confirmed by MS (ESI+ve).

### Step 5. Preparation of benzyl (2-(2-(2-(2-(((3aS,4R,7R,7aR)-7-acetamido-4-(azidomethyl)-2,2-dimethyltetrahydro-4H-[1,3]dioxolo[4,5-c]pyran-6-yl)oxy)ethoxy)ethoxy)ethoxy)-ethyl) carbamate 639

((3aR,4R,7R,7aR)-7-acetamido-2,2-dimethyl-6-((3-oxo-1-phenyl-2,7,10,13-tetraoxa-4-azapentadecan-15-yl)oxy)tetrahydro-4H-[1,3]dioxolo[4,5-c]pyran-4-yl)methyl 4-methylbenzenesulfonate (638) (25.0 g, 34.5 mmol) and NaN₃ (28.7 g, 434.6 mmol) were heated in DMSO/H₂O (200 ml/20 ml) at 100° C. for 12 h. The reaction was cooled and partitioned between EtOAc and saturated NaHCO₃. The aqueous was further extracted another two times and the combined organics washed with saturated NaHCO₃, water and brine, dried (Na₂SO₄) and concentrated in-vacuo. The residue was purified by automated flash chromatography (5% MeOH/DCM) to give benzyl (2-(2-(2-(2-(((3aS,4R,7R,7aR)-7-acetamido-4-(azidomethyl)-2,2-dimethyltetrahydro-4H-[1,3]dioxolo[4,5-c]pyran-6-yl)oxy)ethoxy)ethoxy)ethoxy)ethyl) carbamate (639) (16.1 g, 78.2%). Product confirmed by MS (ESI+ve).

### Step 6. Preparation of benzyl (2-(2-(2-(2-(((3aS,4R,7R,7aR)-7-acetamido-4-((4-(3-methoxyphenyl)-1H-1,2,3-triazol-1-yl)methyl)-2,2-dimethyltetrahydro-4H-[1,3]dioxolo[4,5-c]pyran-6-yl)oxy)ethoxy)ethoxy)ethoxy)ethyl)carbamate 640

Benzyl (2-(2-(2-(2-(((3aS,4R,7R,7aR)-7-acetamido-4-(azidomethyl)-2,2-dimethyltetrahydro-4H-[1,3]dioxolo[4,5-c]pyran-6-yl)oxy)ethoxy)ethoxy)ethoxy)ethyl)carbamate (39) (16.1 g, 27.0 mmol) was stirred in MeOH (200 ml) at RT. 1-Ethynyl-3-methoxybenzene (4.28 g, 32.4 mmol), tris(benzyltriazolylmethyl)amine (0.72 g, 1.35 mmol), CuSO₄ (0.07 g, 0.27 mmol in 1 ml H₂O) and sodium ascorbate (0.53 g, 2.7 mmol in 5 ml H₂O) were added sequentially and the reaction stirred at RT for 16 h. The solvent was removed in-vacuo, the residue taken up in DCM (200 ml) and washed with water. The aqueous layer was back extracted with DCM and the combined organics washed with brine and dried (Na₂SO₄). The reaction was concentrated in-vacuo and the residue purified by automated flash chromatography (10% MeOH/EtOAc) to give benzyl (2-(2-(2-(2-(((3aS,4R,7R,7aR)-7-acetamido-4-((4-(3-methoxyphenyl)-1H-1,2,3-triazol-1-yl)methyl)-2,2-dimethyltetrahydro-4H-[1,3]dioxolo[4,5-c]pyran-6-yl)oxy)ethoxy)ethoxy)ethoxy)ethyl) carbamate (640) (15.0 g, 76.4%). Product confirmed by MS (ESI+ve).

### Step 7. Preparation of benzyl (2-(2-(2-(2-(((3R,4R,5R,6R)-3-acetamido-4,5-dihydroxy-6-((4-(3-methoxyphenyl)-1H-1,2,3-triazol-1-yl)methyl)tetrahydro-2H-pyran-2-yl)oxy)ethoxy) ethoxy)ethoxy)ethyl)carbamate 641

Benzyl (2-(2-(2-(2-(((3aS,4R,7R,7aR)-7-acetamido-4-((4-(3-methoxyphenyl)-1H-1,2,3-triazol-1-yl)methyl)-2,2-dimethyltetrahydro-4H-[1,3]dioxolo[4,5-c]pyran-6-yl)oxy)ethoxy)ethoxy) ethoxy)ethyl)carbamate (640) (15.0 g, 20.6 mmol) was stirred in MeCN (200 ml) and 1.84% H₂SO₄ (180 ml) at RT for 96 h. The reaction was extracted with EtOAc (3×250 ml), washed with saturated NaHCO₃, water and brine, dried (Na₂SO₄) and concentrated in-vacuo to give benzyl (2-(2-(2-(2-(((3R,4R,5R,6R)-3-acetamido-4,5-dihydroxy-6-((4-(3-methoxyphenyl)-1H-1,2,3-triazol-1-yl)methyl)tetrahydro-2H-pyran-2-yl)oxy)ethoxy)ethoxy)ethoxy)ethyl)-carbamate (641) (11.0 g, 16.0 mmol). the product was used in crude in subsequent reactions. Product confirmed by MS (ESI+ve).

### Step 8. Preparation of (2R,3S,4R,5R)-5-acetamido-2-((4-(3-methoxyphenyl)-1H-1,2,3-triazol-1-yl)methyl)-6-((3-oxo-1-phenyl-2,7,10,13-tetraoxa-4-azapentadecan-15-yl)oxy)tetrahydro-2H-pyran-3,4-diyl diacetate 642

Benzyl (2-(2-(2-(2-(((3R,4R,5R,6R)-3-acetamido-4,5-dihydroxy-6-((4-(3-methoxyphenyl)-1H-1,2,3-triazol-1-yl)methyl)tetrahydro-2H-pyran-2-yl)oxy)ethoxy)ethoxy)ethoxy)-ethyl)carbamate (641) (11.0 g, 16.0 mmol) was stirred in pyridine (200 ml) at RT. Acetic anhydride (16.3 g, 160 mmol) was added and the reaction stirred for 16 h at RT followed by 50° C. for 3 h. The reaction was poured over water and extracted three times with DCM (250 ml). The combined organics were washed with saturated NaHCO₃ (×2), IN HCl (×2), water and brine, dried (Na₂SO₄) and concentrated in-vacuo. The residue was purified by automated flash chromatography (5% MeOH/DCM) to give (2R,3S,4R,5R)-5-acetamido-2-((4-(3-methoxyphenyl)-1H-1,2,3-triazol-1-yl)methyl)-6-((3-oxo-1-phenyl-2,7,10,13-tetraoxa-4-azapentadecan-15-yl)oxy)tetrahydro-2H-pyran-3,4-diyl diacetate (642) (10.7 g, 86.7%). Product confirmed by MS (ESI+ve).

### Step 9. Preparation of (2R,3S,4R,5R)-5-acetamido-2-((4-(3-methoxyphenyl)-1H-1,2,3-triazol-1-yl)methyl)-6-((1,1,1-trifluoro-2-oxo-6,9,12-trioxa-314-azatetradecan-14-yl)oxy)tetrahydro-2H-pyran-3,4-diyl diacetate 643

(2R,3S,4R,5R)-5-Acetamido-2-((4-(3-methoxyphenyl)-1H-1,2,3-triazol-1-yl)methyl)-6-((3-oxo-1-phenyl-2,7,10,13-tetraoxa-4-azapentadecan-15-yl)oxy)tetrahydro-2H-pyran-3,4-diyl diacetate (642) (9.06 g, 11.74 mmol) and TFA (1.47 g, 12.91 mmol) were stirred in MeOH at RT. The reaction was hydrogenated over 10% Pd—C for 1 h. The reaction was filtered through celite and concentrated in-vacuo to give (2R,3S,4R,5R)-5-acetamido-2-((4-(3-methoxyphenyl)-1H-1,2,3-triazol-1-yl)methyl)-6-((1,1,1-trifluoro-2-oxo-6,9,12-trioxa-314-azatetradecan-14-yl)oxy)tetrahydro-2H-pyran-3,4-diyl diacetate (643) (8.8 g, 99.7%) which was used in subsequent reactions without purification. Product confirmed by MS (ESI+ve).

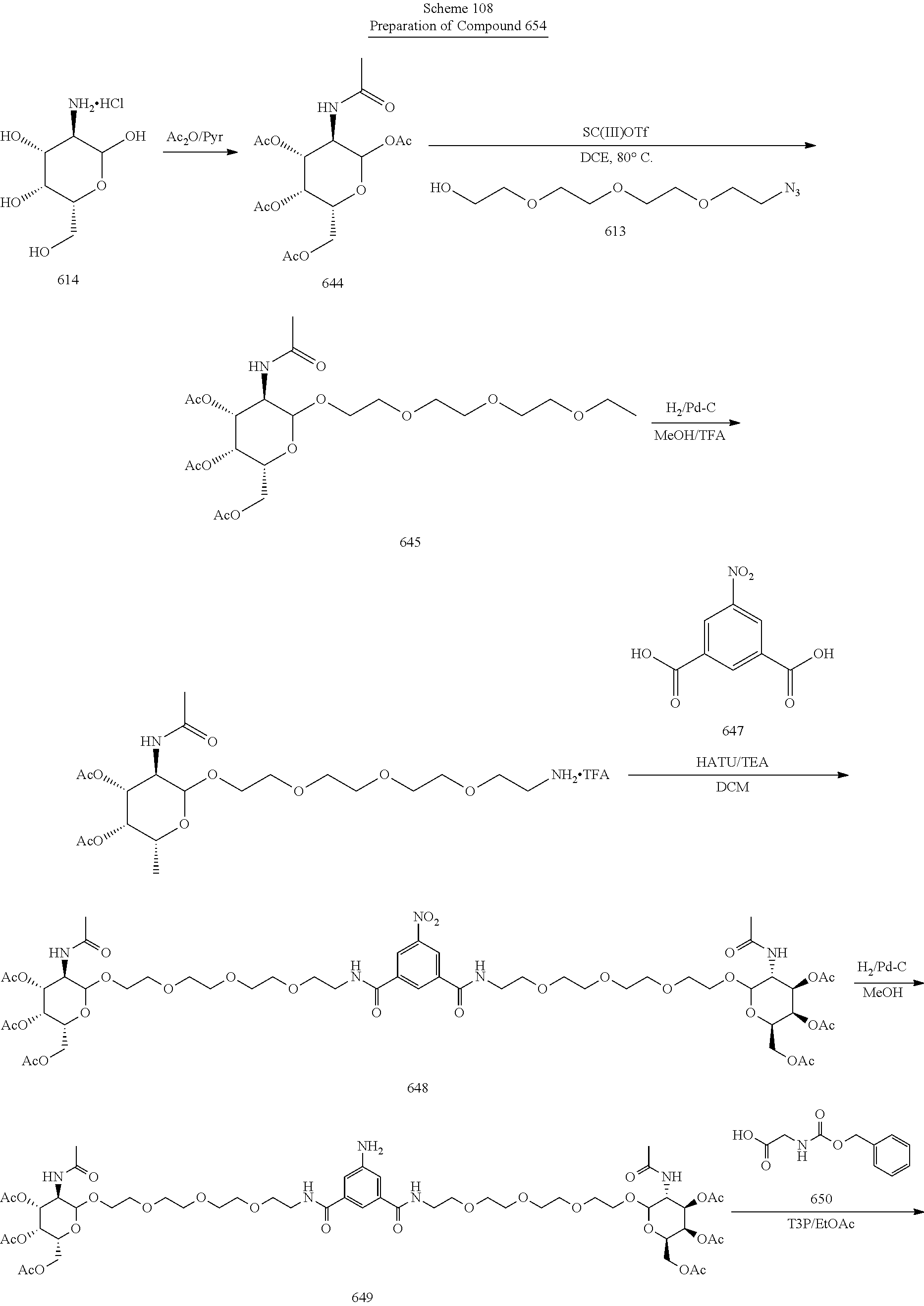

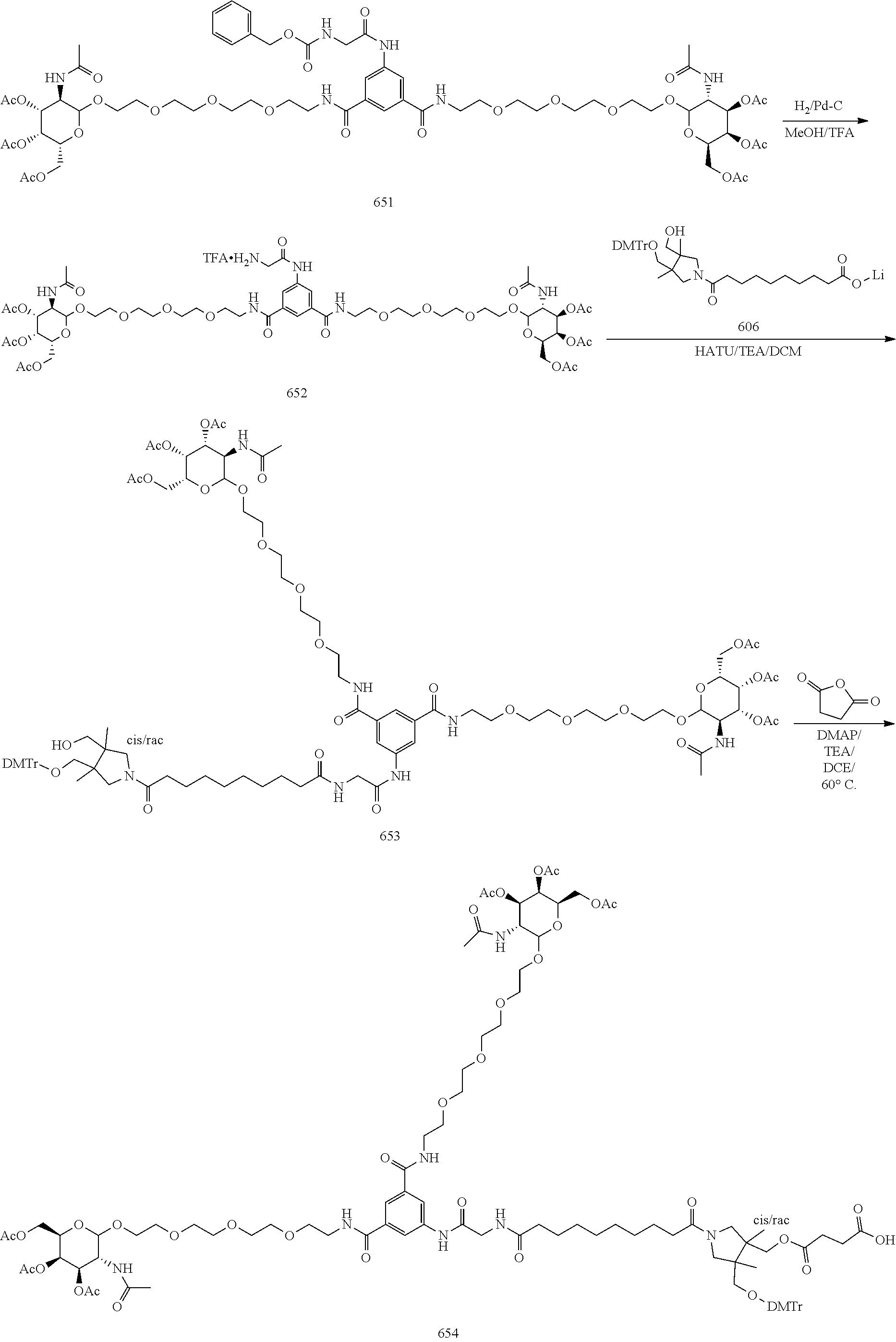

### Step 1. Preparation of Peracetylated Galactosamine 644

D-Galactosamine hydrochloride (614) (250 g, 1.16 mol) in pyridine (1.5 L) was treated with acetic anhydride (1.25 L, 13.2 mol) over 45 minutes. After stirring overnight the reaction mixture was divided into three 1 L portions. Each 1 L portion was poured into 3 L of ice water and mixed for one hour. After mixing the solids were filtered off, combined, frozen over liquid nitrogen and then lyophilized for five days to yield peracetylated galactosamine (644) (369.4 g, 82%) as a white solid. Rf (0.58, 10% MeOH—CH₂Cl₂).

### Step 2. Preparation of (2R,3R,4R,5R,6R)-5-acetamido-2-(acetoxymethyl)-6-(2-(2-(2-(2-azidoethoxy)ethoxy)ethoxy)ethoxy)tetrahydro-2H-pyran-3,4-diyl diacetate 645

Peracetylated galactosamine (644) (25 g, 64.21 mmol) was heated with scandium triflate (1.58 g, 3.21 mmol) in dry DCE at 90° C. for 3 hours. The reaction was cooled to RT, quenched with 5 ml TEA and concentrated in-vacuo. The residue was purified by automated column chromatography (2-10% MeOH/DCM) to give (2R,3R,4R,5R)-5-acetamido-2-(acetoxymethyl)-6-(2-(2-(2-(2-azidoethoxy)ethoxy)ethoxy)ethoxy)tetrahydro-2H-pyran-3,4-diyl diacetate (645) (27 g, 76.5%). Product confirmed by MS.

### Step 3. Preparation of 2-(2-(2-(2-(((2R,3R,4R,5R,6R)-3-acetamido-4,5-diacetoxy-6-(acetoxymethyl)tetrahydro-2H-pyran-2-yl)oxy)ethoxy)ethoxy)ethoxy)ethan-1-aminium 2,2,2-trifluoroacetate 646

A solution of the azide 645 (7.12 g, 13 mmol) in EtOAc (150 ml) and trifluoroacetic acid (2 ml) was treated with palladium on charcoal (1.5 g, 10% w/w wet basis). The reaction mixture was then purged with hydrogen and stirred vigorously overnight. After purging with nitrogen, the mixture was filtered through Celite, rinsing with MeOH. 6Rf (0.34, 15% MeOH—CH₂Cl₂).

### Step 4. Preparation of (2R,2′R,3R,3′R,4R,4′R,5R,5′R)-(((5-nitro-1,3-phenylene)bis(1-oxo-5,8,11-trioxa-2-azatridecane-1,13-diyl))bis(oxy))bis(5-acetamido-2-(acetoxymethyl) tetrahydro-2H-pyran-6,3,4-triyl) tetraacetate 648

(2R,3R,4R,5R)-5-Acetamido-2-(acetoxymethyl)-6-((1,1,1-trifluoro-2-oxo-6,9,12-trioxa-314-azatetradecan-14-yl)oxy)tetrahydro-2H-pyran-3,4-diyl diacetate (646) (13.25 g, 20.84 mmol), 5-nitroisophthalic acid (647) (2.0 g, 9.5 mmol), HATU (12.3 g, 32.21 mmol) and TEA (5.75 g, 59.0 mmol) were stirred in DCM at RT for 16 h. The reaction was diluted with DCM, washed sequentially with 1M HCl, saturated NaHCO₃, water and brine, dried over Na₂SO₄ and concentrated in-vacuo. The residue was purified by automated flash chromatography (5% MeOH/DCM) to give (2R,2′R,3R,3′R,4R,4′R,5R,5′R)-(((5-nitro-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (648) (4.43 g, 38.3%). Product confirmed by MS (ESI+ve).

### Step 5. Preparation of (2R,2′R,3R,3′R,4R,4′R,5R,5′R)-(((5-amino-1,3-phenylene)bis(1-oxo-5,8,11-trioxa-2-azatridecane-1,13-diyl))bis(oxy))bis(5-acetamido-2-(acetoxymethyl)tetra hydro-2H-pyran-6,3,4-triyl) tetraacetate 649

(2R,2′R,3R,3′R,4R,4′R,5R,5′R)-(((5-Nitro-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (648) (26.1 g, 23.05 mmol) was stirred in MeOH at RT. The reaction was hydrogenated over 10% Pd—C (2.6 g) at RT for 2 hours. The reaction was filtered through celite and concentrated in-vacuo to give (2R,2R,3R,3′R,4R,4′R,5R,5′R)-(((5-amino-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (649) (28.0 g, 99.9%) which was used in subsequent reactions without further purification. Product confirmed by MS (ESI+ve).

### Step 6. Preparation of (2R,3R,4R,5R)-5-acetamido-6-((1-(3-((2-(2-(2-(2-(((3R,4R,5R,6R)-3-acetamido-5-acetoxy-6-(acetoxymethyl)-4-hydroxytetrahydro-2H-pyran-2-yl)oxy)ethoxy) ethoxy)ethoxy)ethyl)carbamoyl)-5-(2-(((benzyloxy)carbonyl)amino) acetamido)phenyl)-1-oxo-5,8,11-trioxa-2-azatridecan-13-yl)oxy)-2-(acetoxymethyl)tetrahydro-2H-pyran-3,4-diyl diacetate 651

(2R,2′R,3R,3′R,4R,4′R,5R,5′R)-(((5-Amino-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (649) (0.5 g, 0.45 mmol) and CBZ-gly (650) (0.09 g, 0.45 mmol) were stirred in EtOAc at RT. T3P (50% solution in EtOAc) (0.29 g, 0.91 mmol) was added and the reaction stirred at RT O/N. Additional T3P (0.3 eq) added and the reaction stirred for a further 1 h. The reaction was washed with saturated NaHCO₃ and brine, dried (Na₂SO₄), concentrated in-vacuo and the residue purified by automated flash chromatography (10% MeOH/DCM) to give (2R,3R,4R,5R)-5-acetamido-6-((1-(3-((2-(2-(2-(2-(((3R,4R,5R,6R)-3-acetamido-5-acetoxy-6-(acetoxymethyl)-4-hydroxytetrahydro-2H-pyran-2-yl)oxy)ethoxy)ethoxy)-ethoxy)ethyl)carbamoyl)-5-(2-(((benzyloxy)carbonyl)amino)acetamido)phenyl)-1-oxo-5,8,11-trioxa-2-azatridecan-13-yl)oxy)-2-(acetoxymethyl)tetrahydro-2H-pyran-3,4-diyl diacetate (651) (0.33 g, 56.8%). Product confirmed by MS (ESI+ve).

### Step 7. Preparation of (2R,2′R,3R,3′R,4R,4′R,5R,5′R)-(((5-(2-((2,2,2-trifluoroacetyl)-14-azaneyl)acetamido)-1,3-phenylene)bis(1-oxo-5,8,11-trioxa-2-azatridecane-1,13-diyl))bis (oxy))bis(5-acetamido-2-(acetoxymethyl)tetrahydro-2H-pyran-6,3,4-triyl) tetraacetate 652

(2R,3R,4R,5R)-5-Acetamido-6-((1-(3-((2-(2-(2-(2-(((3R,4R,5R,6R)-3-acetamido-5-acetoxy-6-(acetoxymethyl)-4-hydroxytetrahydro-2H-pyran-2-yl)oxy)ethoxy)ethoxy)ethoxy)ethyl) carbamoyl)-5-(2-(((benzyloxy)carbonyl)amino)acetamido)phenyl)-1-oxo-5,8,11-trioxa-2-azatridecan-13-yl)oxy)-2-(acetoxymethyl)tetrahydro-2H-pyran-3,4-diyl diacetate (651) (3.3 g, 2.39 mmol) and TFA (0.29 g, 2.51 mmol) were stirred in MeOH at RT. The reaction was hydrogenated over 10% Pd—C (400 mg) for two h., filtered through celite and concentrated in-vacuo to give (2R,2′R,3R,3′R,4R,4′R,5R,5′R)-(((5-(2-((2,2,2-trifluoroacetyl)-14-azaneyl)-acetamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (652) (3.21 g, 98.7%) which was used in subsequent reactions without further purification. Product confirmed by MS (ESI+ve).

### Step 8. Preparation of (2R,2′R,3R,3′R,4R,4′R,5R,5′R)-(((5-(2-(10-(3-((bis(4-methoxyphenyl) (phenyl)methoxy)methyl)-4-(hydroxymethyl)-3,4-dimethylpyrrolidin-1-yl)-10-oxodecanamido)acetamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate 653

(2R,2R,3R,3′R,4R,4′R,5R,5′R)-(((5-(2-((2,2,2-Trifluoroacetyl)-14-azaneyl)acetamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (652×1.0 g, 0.73 mmol), lithium 10-(3-((bis(4-methoxyphenyl)(phenyl)methoxy)methyl)-4-(hydroxymethyl)-3,4-dimethyl-pyrrolidin-1-yl)-10-oxodecanoate (606) (0.45 g, 0.73 mmol), HATU (0.47 g, 1.25 mmol) and TEA (0.22 g, 2.2 mmol) were stirred in DCM at RT for 4 h. The reaction was diluted with DCM and washed sequentially with saturated NaHCO₃, water and brine, dried (Na₂SO₄) and concentrated in-vacuo. The residue was purified by automated flash chromatography (5% MeOH/DCM) to give (2R,2′R,3R,3′R,4R,4′R,5R,5′R)-(((5-(2-(10-(3-((bis(4-methoxy-phenyl)(phenyl)methoxy)methyl)-4-(hydroxymethyl)-3,4-dimethylpyrrolidin-1-yl)-10-oxodecanamido)acetamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (653) (1.02 g, 75.2%). Product confirmed by MS (ESI+ve).

### Step 9. Preparation of 4-((1-(10-((2-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid 654

(2R,2R,3R,3′R,4R,4′R,5R,5′R)-(((5-(2-(10-(3-((Bis(4-methoxyphenyl)(phenyl)methoxy)-methyl)-4-(hydroxymethyl)-3,4-dimethylpyrrolidin-1-yl)-10-oxodecanamido)acetamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (653) (1.05 g, 0.57 mmol), succinic anhydride (0.28 g, 2.84 mmol), DMAP (0.35 g, 2.84 mmol) and TEA (0.58 g, 5.68 mmol) were heated in dry DCE at 60° C. for 2 hours. MeOH (5 ml) was added and the reaction stirred for a further 30 mins then cooled and concentrated in-vacuo. The residue was taken up in DCM and washed sequentially with saturated NaHCO₃ (×4), water and brine. The organics were dried (Na₂SO₄), and concentrated in-vacuo to give 4-((1-(10-((2-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid (654) (1.1 g, 99.4%) which was used as a crude product in subsequent reactions. Product confirmed by MS (ESI+ve).

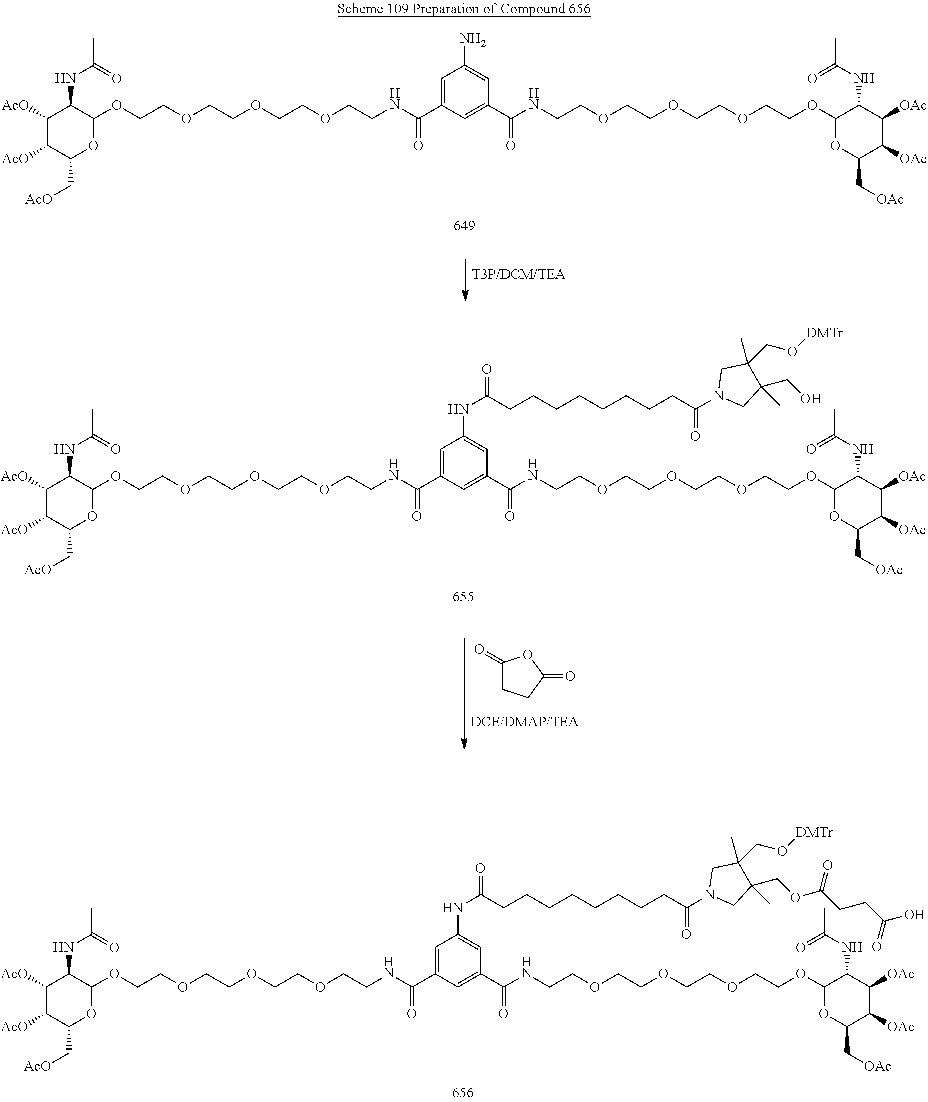

### Step 1. Preparation of (2R,2′R,3,3′R,4R,4′R,5R,5′R)-(((5-(10-(3-((bis(4-methoxy-phenyl)(phenyl)methoxy)methyl)-4-(hydroxymethyl)-3,4-dimethylpyrrolidin-1-yl)-10-oxodecanamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate 655

(2R,2′R,3R,3′R,4R,4′R,5R,5′R)-(((5-Amino-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (649) (48 g, 3.36 mmol), lithium 10-(3-((bis(4-methoxyphenyl)-(phenyl)methoxy) methyl)-4-(hydroxymethyl)-3,4-dimethylpyrrolidin-1-yl)-10-oxodecanoate (6) (2.13 g, 3.36 mmol), TEA (1 ml, 6.7 mmol) and T3P (50% W/W solution in EtOAc) (4.3 g, 6.72 mmol) were stirred in DCM at RT for 16 h. The reaction was washed sequentially with saturated NaHCO₃, water and brine, dried (Na₂SO₄) and concentrated in-vacuo. The residue was purified by automated flash chromatography (10/a MeOH/DCM) to give 2R,2′R,3R,3′R, 4R,4′R,5R,5′R)-(((5-(10-(3-((bis(4-methoxyphenyl)(phenyl)methoxy)methyl)-4-(hydroxyl-methyl)-3,4-dimethylpyrrolidin-1-yl)-10-oxodecanamido)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (655) (1.37 g, 22.5%). Product confirmed by MS (ESI+ve).

### Step 2. Preparation of 4-((1-(10-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl) carbamoyl)phenyl)amino)-10-oxodecanoyl)-4-((bis(4-methoxyphenyl)(phenyl)methoxy) methyl)-3,4-dimethylpyrrolidin-3-yl)methoxy)-4-oxobutanoic acid 656

This compound was prepared in an analogous manner to 4-((1-(10-((2-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid (654)

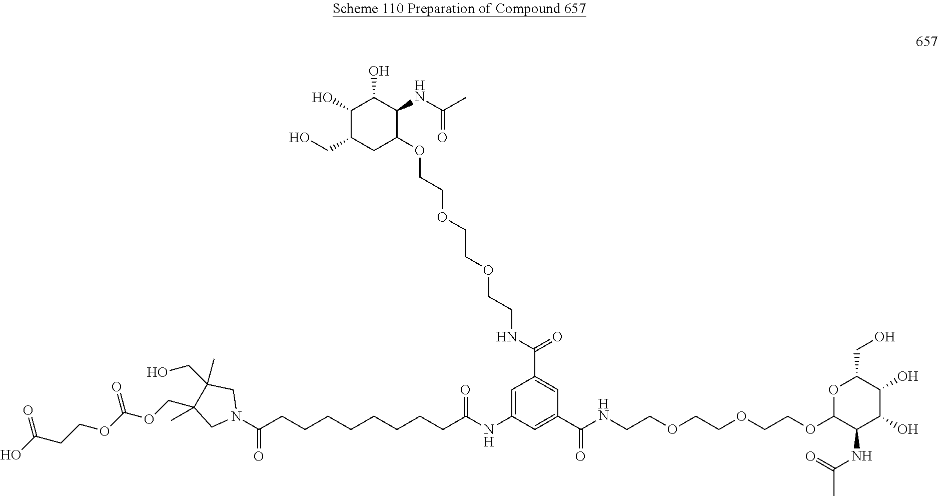 [SMILES: CC(=O)N[C@H]1C(OCCOCCOCCNC(=O)c2cc(NC(=O)CCCCCCCCC(=O)N3CC(C)(CO)C(C)(COC(=O)OCCC(=O)O)C3)cc(C(=O)NCCOCCOCCOC3C[C@H](CO)[C@H](O)[C@H](O)[C@H]3NC(C)=O)c2)O[C@H](CO)[C@H](O)[C@@H]1O]

### Synthesis of 3-((((1-(10-((3,5-bis((2-(2-(2-(((3R,4R,5R,6R)-3-acetamido-4,5-dihydroxy-6-(hydroxymethyl)tetrahydro-2H-pyran-2-yl)oxy)ethoxy)ethoxy)ethyl)carbamoyl) phenyl)amino)-10-oxodecanoyl)-4-(hydroxymethyl)-3,4-dimethylpyrrolidin-3-yl)methoxy)carbonyl)oxy)propanoic acid 657

This compound was prepared in an analogous manner to 4-((1-(10-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)carbamoyl)phenyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid (654)

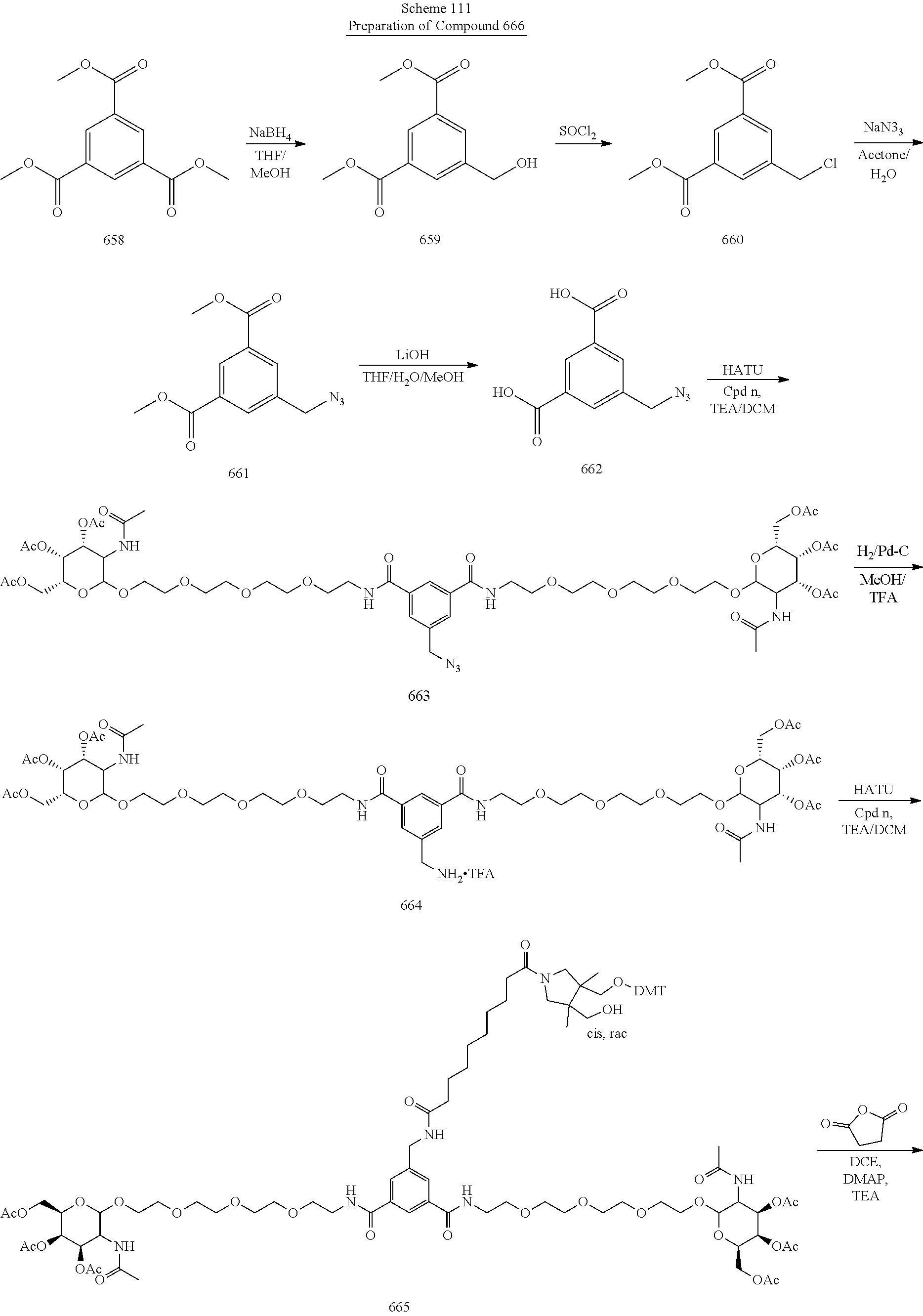

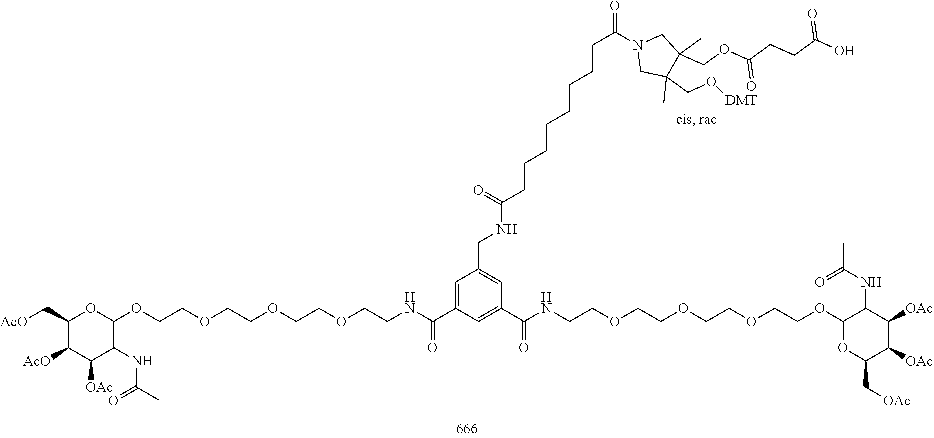

### Step 1. Preparation of dimethyl 5-(hydroxymethyl)isophthalate 659

Trimethyl benzene-1,3,5-tricarboxylate (658) (40 g, 159 mmol) and NaBH₄ were stirred in THF at RT. MeOH (30 ml) in THF (120 ml) was added dropwise slowly. After complete addition the reaction was refluxed for 30 mins. After cooling the reaction was quenched with 1M HCl and extracted into EtOAc. The organics were washed sequentially with 1M HCl, NaHCO₃, water and brine, dried (Na₂SO₄) and concentrated in-vacuo. The residue was purified by automated flash chromatography (50/50 EtOAc/hex) to give dimethyl 5-(hydroxymethyl)isophthalate (659) (20.5 g, 53.2%). ¹H NMR (400 MHz, CDCl₃) δ 8.59 (s, 1H), 8.23 (s, 2H), 4.81 (s, 2H), 3.95 (s, 6H). Product confirmed by MS (ESI+ve).

### Step 2. Preparation of dimethyl 5-(chloromethyl)isophthalate 660

Dimethyl 5-(hydroxymethyl)isophthalate (659) (20.5 g, 80.5%) was refluxed in SOCl₂ (11.1 g, 94 mmol) for 1.5 h. The reaction was cooled, diluted with DCM and washed sequentially with 0.1 M NaOH (×2), water and brine, dried (Na₂SO₄) and concentrated in-vacuo. The residue was purified by automated flash chromatography (20% EtOAc/Hex) to give dimethyl 5-(chloromethyl)isophthalate (660) (10.84 g, 53%). ¹H NMR (400 MHz, CDCl₃) δ 8.65 (s, 1H), 8.27 (s, 2H), 4.66 (s, 2H), 3.97 (s, 6H). Product confirmed by MS (ESI+ve).

### Step 3. Preparation of dimethyl 5-(azidomethyl)isophthalate 661

Dimethyl 5-(chloromethyl)isophthalate (660) (10.84 g, 45 mmol) and NaN₃ (18 g, 270 mmol) were refluxed in acetone/water (3/1) for 16 h. The reaction was cooled, concentrated in-vacuo and the residue taken up in DCM. The organics were washed with water and brine, dried (Na₂SO₄) and concentrated in-vacuo. The residue was purified by flash chromatography (15% EtOAc/Hex) to give dimethyl 5-(azidomethyl)isophthalate (661) (9.84 g, 88%). ¹H NMR (400 MHz, CDCl₃) δ 8.66 (s, 2H), 8.2 (s, 2H), 4.49 (s, 2H), 3.97 (s, 2H). Product confirmed by MS (ESI+ve).

### Step 4. Preparation of 5-(azidomethyl)isophthalic acid 662

Dimethyl 5-(azidomethyl)isophthalate (661) (9.84 g, 39.5 mmol) and LiOH (2.1 g, 87 mmol) were stirred in THF/H₂O/MeOH at RT for 48 h. The organic solvent was removed in-vacuo and the residue acidified with 1M HCl. The aqueous was extracted with EtOAc (×3) and the combined organics dried (Na₂SO₄) and concentrated in-vacuo to give 5-(azidomethyl)isophthalic acid (662) (8.0 g, 91.6%) which was used in subsequent reactions without further purification

### Step 5. Preparation of (2R,2′R,3R,3′R,4R,4′R)-(((5-(azidomethyl)-1,3-phenylene)bis(1-oxo-5,8,11-trioxa-2-azatridecane-1,13-diyl))bis(oxy))bis(5-acetamido-2-(acetoxymethyl) tetrahydro-2H-pyran-6,3,4-triyl) tetraacetate 663

5-(Azidomethyl)isophthalic acid (662) (4.42 g, 20 mmol), 2-(2-(2-(2-(((2R,3R,4R,5R,6R)-3-acetamido-4,5-diacetoxy-6-(acetoxymethyl)tetrahydro-2H-pyran-2-yl)oxy)ethoxy)ethoxy) ethoxy)ethan-1-aminium 2,2,2-trifluoroacetate (646) (25 g, 40 mmol), HATU (24.4 g, 64 mmol) and TEA (17 ml, 120 mmol) were stirred in DCM at RT for 16h. The reaction was washed sequentially with 1M HCl, saturated NaHCO₃, water and brine, dried (Na₂SO₄) and concentrated in-vacuo. The residue was purified by automated flash chromatography (7% MeOH/DCM) to give (2R,2′R,3R,3′R,4R,4′R)-(((5-(azidomethyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (663) (10.9 g, 44.5%). Product confirmed by MS (ESI+ve).

### Step 6. Preparation of (2R,2′R,3R,3′R,4R,4′R)-(((5-(((2,2,2-trifluoroacetyl)-14-azaneyl)methyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate 664

(2R,2′R,3R,3′R,4R,4′R)-(((5-(Azidomethyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (663) (10.9 g, 8.9 mmol) and TFA (0.68 ml, 8.9 mmol) were stirred in MeOH at RT. The reaction was hydrogenated over 10% Pd—C for 1 h. The reaction was filtered through celite, concentrated in-vacuo and the residue purified by automated flash chromatography (15% MeOH/DCM) to give (2R,2′R,3R,3′R,4R,4′R)-(((5-(((2,2,2-trifluoroacetyl)-14-azaneyl)methyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (664) (6.41 g, 54.7%). Product confirmed by MS (ESI+ve).

### Step 7. Preparation of (2R,2′R,3R,3′R,4R,4′R)-(((5-((10-(3-((bis(4-methoxyphenyl)(phenyl) methoxy)methyl)-4-(hydroxymethyl)-3,4-dimethylpyrrolidin-1-yl)-10-oxodecanamido) methyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate 665

(2R,2′R,3R,3′R,4R,4′R)-(((5-(((2,2,2-Trifluoroacetyl)-14-azaneyl)methyl)-1,3-phenylene)bis(1-oxo-5,8,11-trioxa-2-azatridecane-1,13-diyl))bis(oxy))bis(5-acetamido-2-(acetoxymethyl) tetrahydro-2H-pyran-6,3,4-triyl) tetraacetate (3.0 g, 2.3 mmol), lithium 10-(3-((bis(4-methoxyphenyl)(phenyl)methoxy)methyl)-4-(hydroxymethyl)-3,4-dimethylpyrrolidin-1-yl)-10-oxodecanoate (665) (1.5 g, 2.3 mmol), HATU (1.4 g, 3.7 mmol) and TEA (1 ml, 7.0 mmol) were stirred at RT O/N. The reaction was diluted with DCM washed with saturated NaHCO₃, water and brine, dried (Na₂SO₄) and concentrated in-vacuo. The residue was purified by automated flash chromatography (5% MeOH/DCM) to give (2R,2′R,3R,3′R,4R,4R)-(((5-((10-(3-((bis(4-methoxyphenyl)(phenyl)methoxy)methyl)-4-(hydroxymethyl)-3,4-dimethylpyrrolidin-1-yl)-10-oxodecanamido)methyl)-1,3-phenylene)bis(1-oxo-5,8,11-trioxa-2-azatridecane-1,13-diyl))bis(oxy))bis(5-acetamido-2-(acetoxymethyl)tetrahydro-2H-pyran-6,3,4-triyl) tetraacetate (665) (1.8 g, 43.0%). Product confirmed by MS (ESI+ve).

### Step 8. Preparation of 4-((1-(10-((3,5-bis((2-(2-(2-(2-(((4R,5R,6R)-3-acetamido-4,5-diacetoxy-6-(acetoxy methyl)tetrahydro-2H-pyran-2-yl)oxy)ethoxy)ethoxy)-ethoxy)ethyl)carbamoyl)benzyl) amino)-10-oxodecanoyl)-4-((bis(4-methoxyphenyl)(phenyl)methoxy)methyl)-3,4-dimethylpyrrolidin-3-yl)methoxy)-4-oxobutanoic acid 666

This compound was prepared in an analogous manner to 4-((1-(10-((2-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid (654). Product confirmed by MS (ESI+ve).

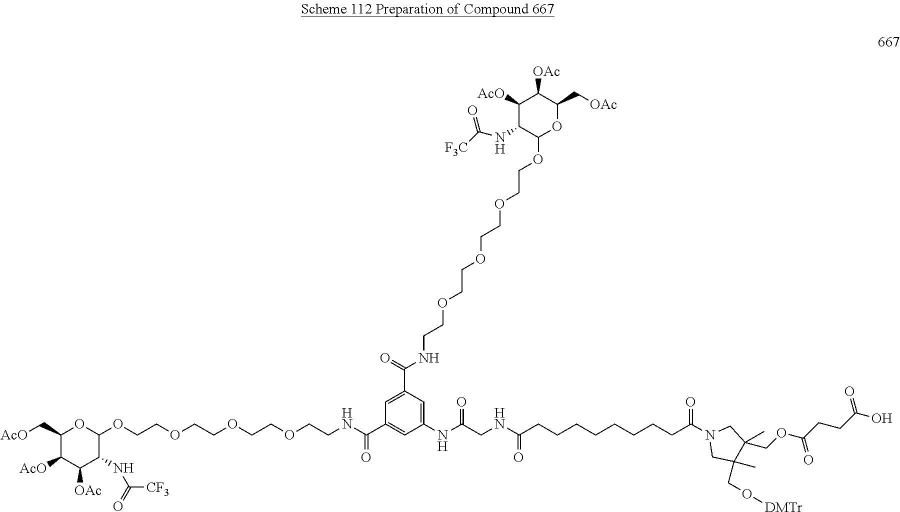

### Synthesis of 4-((1-(10-((2-((3,5-bis((2-(2-(2-(2-(((3R,4R,5R,6R)-4,5-diacetoxy-6-(acetoxy methyl)-3-(2,2,2-trifluoroacetamido)tetrahydro-2H-pyran-2-yl)oxy)ethoxy)ethoxy)ethoxy) ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid 667

This compound was prepared in an analogous fashion to 654 (scheme 8), using (3R,4R,5R,6R)-6-(acetoxymethyl)-3-(2,2,2-trifluoroacetamido)tetrahydro-2H-pyran-2,4,5-triyl triacetate instead of peracetylated galactosamine (606). Product confirmed by MS (ESI+ve).

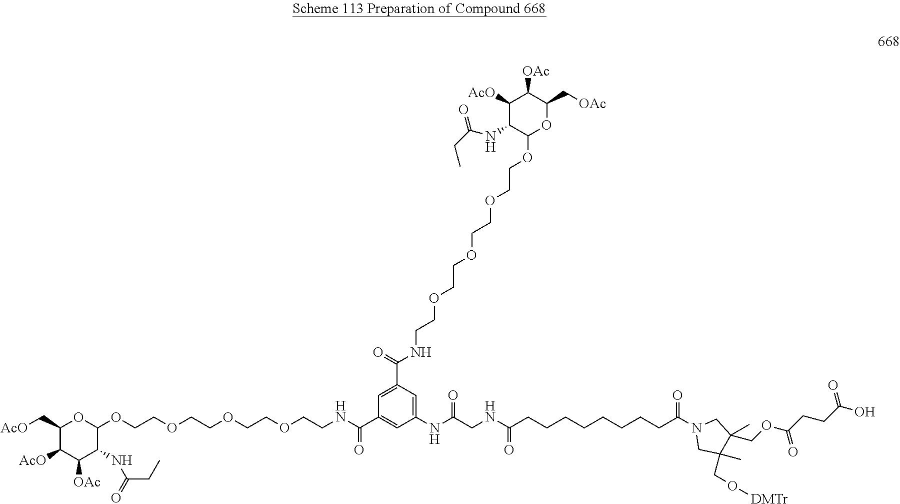

### Synthesis of 4-((1-(10-((2-((3,5-bis((2-(2-(2-(2-(((3R,4R,5R,6R)-4,5-diacetoxy-6-(acetoxy methyl)-3-propionamidotetrahydro-2H-pyran-2-yl)oxy)ethoxy)ethoxy)ethoxy)ethyl) carbamoyl)phenyl)amino)-2-oxoethyl)amino)-10-oxodecanoyl-4-((bis(4-methoxyphenyl) (phenyl)methoxy)methyl)-3,4-dimethylpyrrolidin-3-yl)methoxy-4-oxobutanoic acid 668

This compound was prepared in an analogous fashion to 654 (scheme 8), using (3R,4R,5R,6R)-6-(acetoxymethyl)-3-propionamidotetrahydro-2H-pyran-2,4,5-triyl triacetate (619b) instead of peracetylated galactosamine (644). Product confirmed by MS (ESI+ve).

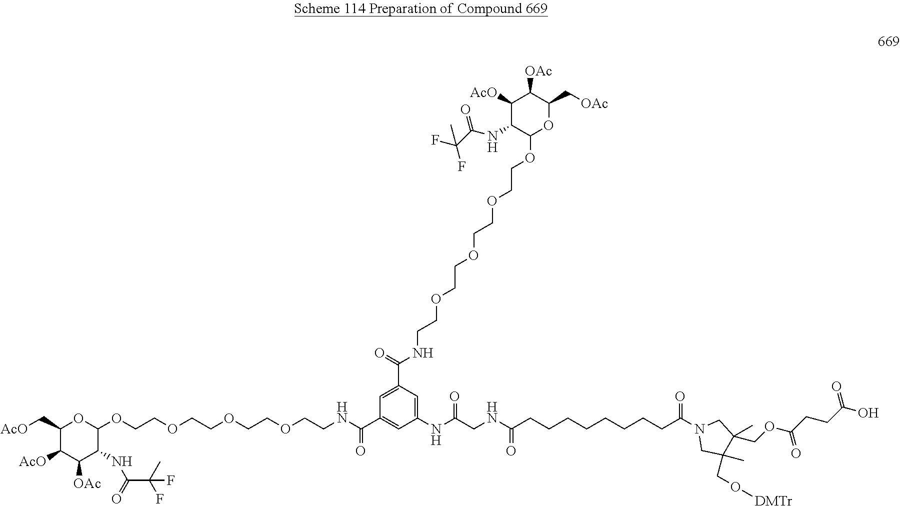

### Synthesis of 4-((1-(10-((2-((3,5-bis((2-(2-(2-(2-(((3R,4R,5R,6R)-4,5-diacetoxy-6-(acetoxy methyl)-3-(2,2-difluoropropanamido)tetrahydro-2H-pyran-2-yl)oxy)ethoxy)ethoxy)-ethoxy) ethyl)carbamoyl)phenyl)amino)-2-oxoethyl)amino)-10-oxodecanoyl)-4-((bis(4-methoxy phenyl)(phenyl)methoxy)methyl)-3,4-dimethylpyrrolidin-3-yl)methoxy)-4-oxobutanoic acid 669

This compound was prepared in an analogous fashion to 654 (scheme 8), using (3R,4R,5R,6R)-6-(acetoxymethyl)-3-(2,2-difluoropropanamido)tetrahydro-2H-pyran-2,4,5-triyl triacetate (619c) instead of peracetylated galactosamine (644). Product confirmed by MS (ESI+ve).

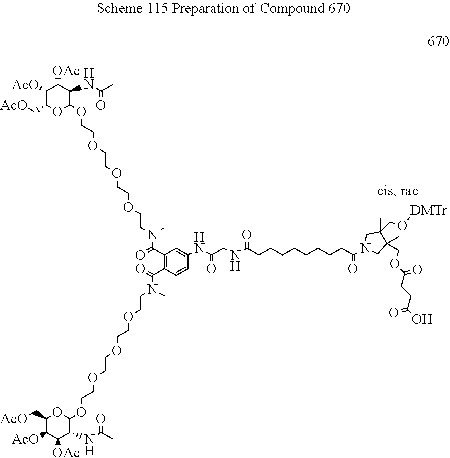

### Synthesis of 4-((1-(10-((2-((3,4-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)ethyl)(methyl) carbamoyl)phenyl)amino)-2-oxoethyl)amino)-10-oxodecanoyl)-4-((bis(4-methoxyphenyl) (phenyl)methoxy)methyl)-3,4-dimethylpyrrolidin-3-yl)methoxy)-4-oxobutanoic acid 670

This compound was prepared in an analogous manner to compound 654 (scheme 8) using (2R,2′R,3R,3′R,4R,4′R,5R,5′R)-(((4-nitro-1,2-phenylene)bis(2-methyl-1-oxo-5′,8′,11′-trioxa-2′-azatridecane-1,13-diyl))bis(oxy))bis(5-acetamido-2-(acetoxymethyl)tetrahydro-2H-pyran-6,3,4-triyl) tetraacetate (634) in place of (2R,2′R,3R,3′R,4R,4′R,5R,5′R)-(((5-nitro-1,3-phenylene)bis(1-oxo-5,8,11-trioxa-2-azatridecane-1,13-diyl))bis(oxy))bis(5-acetamido-2-(acetoxymethyl) tetrahydro-2H-pyran-6,3,4-triyl) tetraacetate (648).

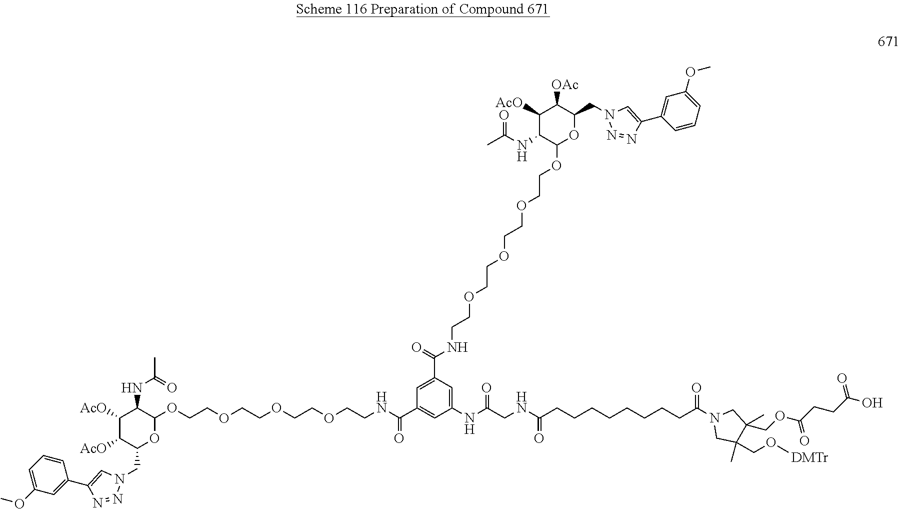

### Synthesis of 4-((1-(10-((2-((3,5-bis((2-(2-(2-(2-(((3R,4R,5S,6R)-3-acetamido-4,5-diacetoxy-6-((4-(3-methoxyphenyl)-1H-1,2,3-triazol-1-yl)methyl)tetrahydro-2H-pyran-2-yl)oxy)ethoxy) ethoxy)ethoxy)ethyl)carbamoyl)phenyl)amino)-2-oxoethyl)amino)-10-oxodecanoyl)-4-((bis(4-methoxyphenyl)(phenyl)methoxy)methyl)-3,4-dimethylpyrrolidin-3-yl)methoxy)-4-oxobutanoic acid 671

This compound was prepared in an analogous manner to compound 654 (scheme 8) using (2R,3S,4R,5R)-5-acetamido-2-((4-(3-methoxyphenyl)-1H-1,2,3-triazol-1-yl)methyl)-6-((1,1,1-trifluoro-2-oxo-6,9,12-trioxa-314-azatetradecan-14-yl)oxy)tetrahydro-2H-pyran-3,4-diyl diacetate (643) in place of 2-(2-(2-(2-(((2R,3R,4R,5R,6R)-3-acetamido-4,5-diacetoxy-6-(acetoxymethyl) tetrahydro-2H-pyran-2-yl)oxy)ethoxy)ethoxy)ethoxy)ethan-1-aminium 2,2,2-trifluoroacetate (646).

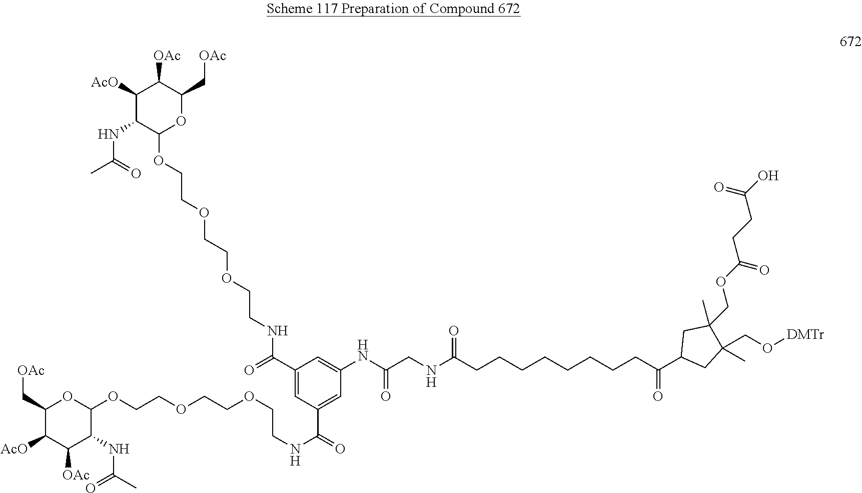

### Synthesis of 4-((4-(10-((2-((3,5-bis((2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethyl)carbamoyl)phenyl) amino)-2-oxoethyl)amino)-10-oxodecanoyl)-2-((bis(4-methoxyphenyl)(phenyl)methoxy) methyl)-1,2-dimethylcyclopentyl)methoxy)-4-oxobutanoic acid 672

This compound was prepared in an analogous manner to compound 654 (scheme 8) using (2R,3R,4R,5R)-5-acetamido-2-(acetoxymethyl)-6-(2-(2-(2-((2,2,2-trifluoroacetyl)-14-azaneyl)ethoxy) ethoxy)ethoxy)tetrahydro-2H-pyran-3,4-diyl diacetate (624) in place of 2-(2-(2-(2-(((2R,3R,4R,5R,6R)-3-acetamido-4,5-diacetoxy-6-(acetoxymethyl)tetrahydro-2H-pyran-2-yl)oxy)ethoxy)ethoxy) ethoxy)ethan-1-aminium 2,2,2-trifluoroacetate (646).

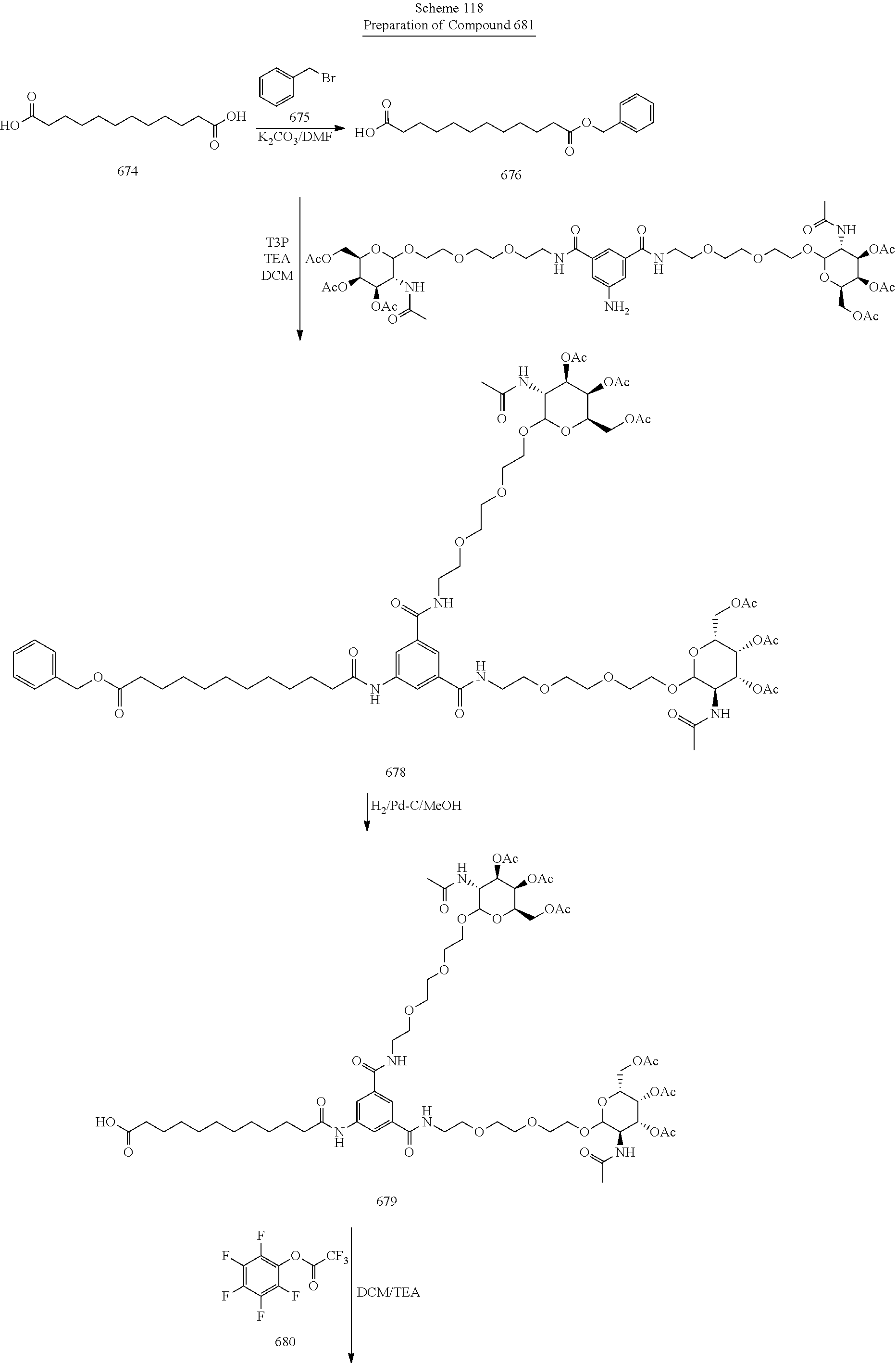

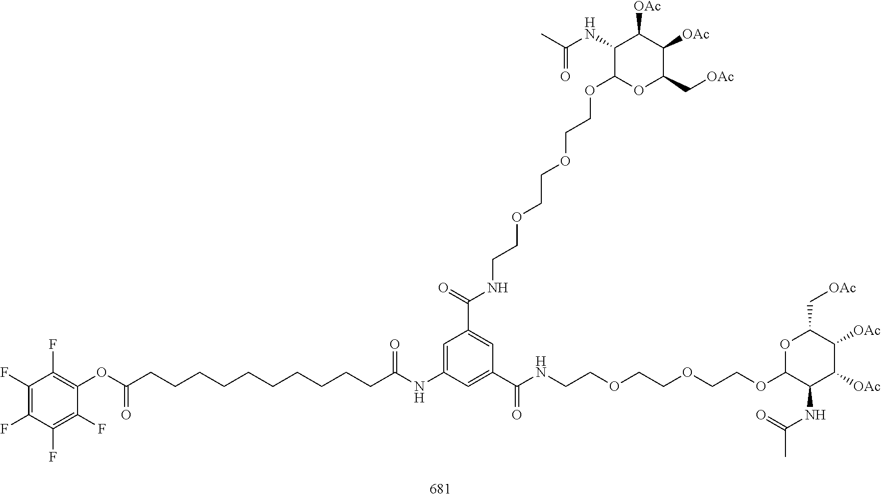 [SMILES: CC(=O)N[C@H]1C(OCCOCCOCCNC(=O)c2cc(NC(=O)CCCCCCCCCCC(=O)Oc3c(F)c(F)c(F)c(F)c3F)cc(C(=O)NCCOCCOCCOC3O[C@H](COC(C)=O)[C@H](OC(C)=O)[C@H](OC(C)=O)[C@H]3NC(C)=O)c2)O[C@H](COC(C)=O)[C@H](OC(C)=O)[C@@H]1OC(C)=O]

### Step 1. Preparation of 12-(benzyloxy)-12-oxododecanoic acid 676

To a solution of dodecanedioic acid (674) (21.0 g, 91.3 mmol) in DMF (200 ml) was added potassium carbonate (10 g, 72.4 mmol) and benzyl bromide (675) (10 ml, 84.2 mmol). The solution was stirred at 80° C. for 4 hours, cooled to 0° C. then carefully acidified with 6M HCl. Dilute with water (250 ml) and extract with ethyl acetate (500 ml). The ethyl acetate extract was washed with brine (3×250 ml), dried on magnesium sulfate, filtered and concentrated to dryness. The solid was suspended in dichloromethane (200 ml) and filtered. The filtrate, which was now enriched in the product, was concentrated then purified by column chromatography on silica gel 60 (Gradient: 0 to 10% methanol in DCM) to afford 12-(benzyloxy)-12-oxododecanoic acid 6 (76) as a colorless solid (13 g, 45%). Structure confirmed by mass spectroscopy

### Step 2. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethyl)carbamoyl)-5-(12-(benzyloxy)-12-oxododecanamido)benzamido)ethoxy)ethoxy) ethoxy)-2-(acetoxymethyl)tetrahydro-2H-pyran-3,4-diyl diacetate 678

To a solution of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)-ethoxy)ethyl) carbamoyl)-5-aminobenzamido)ethoxy)ethoxy)ethoxy)-2-(acetoxymethyl)-tetrahydro-2H-pyran-3,4-diyl diacetate (677) (4.0 g, 3.6 mmol), 12-(benzyloxy)-12-oxododecanoic acid (676) (1.3 g, 4.1 mmol) and triethylamine (1.5 ml, 10.8 mmol) in dichloromethane (75 ml) was added dropwise T3P (4.5 g, −9 ml, 50% solution in ethyl acetate). The solution was stirred overnight at room temperature. Upon completion, the reaction mixture was diluted with dichloromethane and carefully quenched with a saturated solution of sodium bicarbonate (200 ml). The biphasic solution was stirred vigorously for 30 minutes. The DCM layer was separated and the aqueous phase was extracted with dichloromethane (1×100 ml). The combined extracts were dried on magnesium sulfate, filtered and concentrated in vacuo to dryness. The residue was purified by column chromatography on silica gel 60 (Gradient: 0-10% MeOH in DCM) to afford the title compound as a colorless solid (1.5 g, 30%).

### Step 3. Preparation of 12-((3-((2-(2-(2-(((3R,4R,5R,6R)-3-acetamido-4,5-diacetoxy-6-(acetoxy methyl)tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)ethoxy)ethyl)carbamoyl)phenyl)amino)-12-oxododecanoic acid 679

To a solution of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)-ethyl) carbamoyl)-5-(12-(benzyloxy)-12-oxododecanamido)benzamido)ethoxy)ethoxy)-ethoxy)-2-(acetoxymethyl)tetrahydro-2H-pyran-3,4-diyl diacetate (678) (1.5 g, 1.1 mmol) in methanol (25 ml) was added 10% palladium on carbon (wet basis, 150 mg, 10% wt/wt). The solution was sparged with hydrogen gas slowly over 1 hour. Upon completion, the solution was sparged with nitrogen, filtered through celite, and concentrated in vacuo to dryness to afford a colorless solid (1.1 g, 79%).

### Step 4. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy) ethyl)carbamoyl)-5-(12-oxo-12-(perfluorophenoxy)dodecanamido)benzamido)ethoxy) ethoxy)ethoxy)-2-(acetoxymethyl)tetrahydro-2H-pyran-3,4-diyl diacetate 681

To a solution of 12-((3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)ethoxy)ethyl) carbamoyl)phenyl)amino)-12-oxododecanoic acid (679) (0.6 g, 0.46 mmol) and triethylamine (125 μL, 0.92 mmol) in dichloromethane (50 ml) was added pentafluorophenyl trifluoroacetate (680) (150 mg, 1.1 mmol). The solution was stirred for 30 minutes at room temperature then concentrated in vacuo to dryness. The residue was purified by column chromatography on silica gel 60 (gradient: 0 to 10% methanol in dichloromethane) to afford the title compound as a colorless solid (475 mg, 70%). Mass (ESI+) m/z 741.0 (M+2H). 1H NMR (400 MHz, DMSO-d6) δ 10.12 (s, 1H), 8.52 (t, J=5.6 Hz, 2H), 8.14 (d, J=1.4 Hz, 2H), 7.91 (t, J=1.6 Hz, 1H), 7.80 (d, J=9.2 Hz, 2H), 5.21 (d, J=3.4 Hz, 2H), 4.97 (dd, J=11.2, 3.4 Hz, 2H), 4.54 (d, J=8.5 Hz, 2H), 4.06-3.99 (m, 7H), 3.88 (dt, J=11.2, 8.8 Hz, 2H), 3.77 (ddd, J=11.1, 5.6, 3.9 Hz, 2H), 3.62-3.46 (m, 22H), 3.46-3.38 (m, 5H), 2.77 (t, J=7.2 Hz, 2H), 2.31 (t, J=7.4 Hz, 2H), 2.10 (s, 7H), 1.99 (s, 7H), 1.89 (s, 7H), 1.77 (s, 7H), 1.69-1.54 (m, 4H), 1.40-1.20 (m, 14H). Mass (ESI+) m/z 741.0 (M+2H).

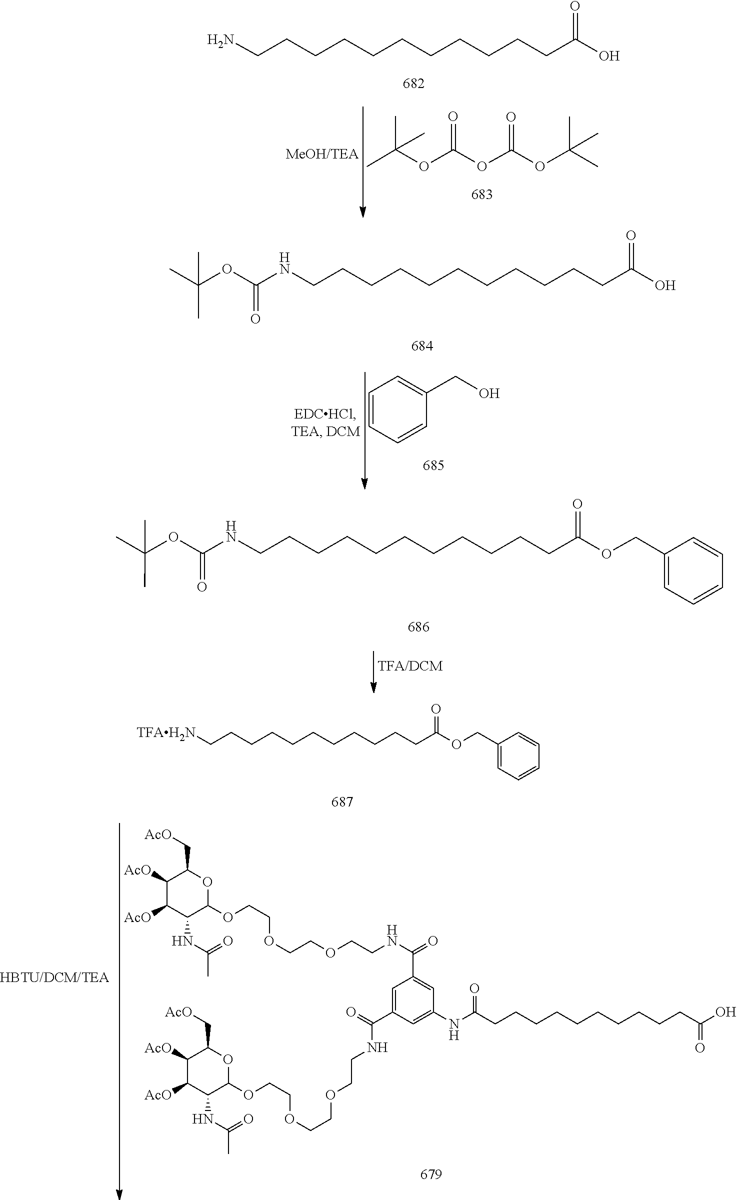

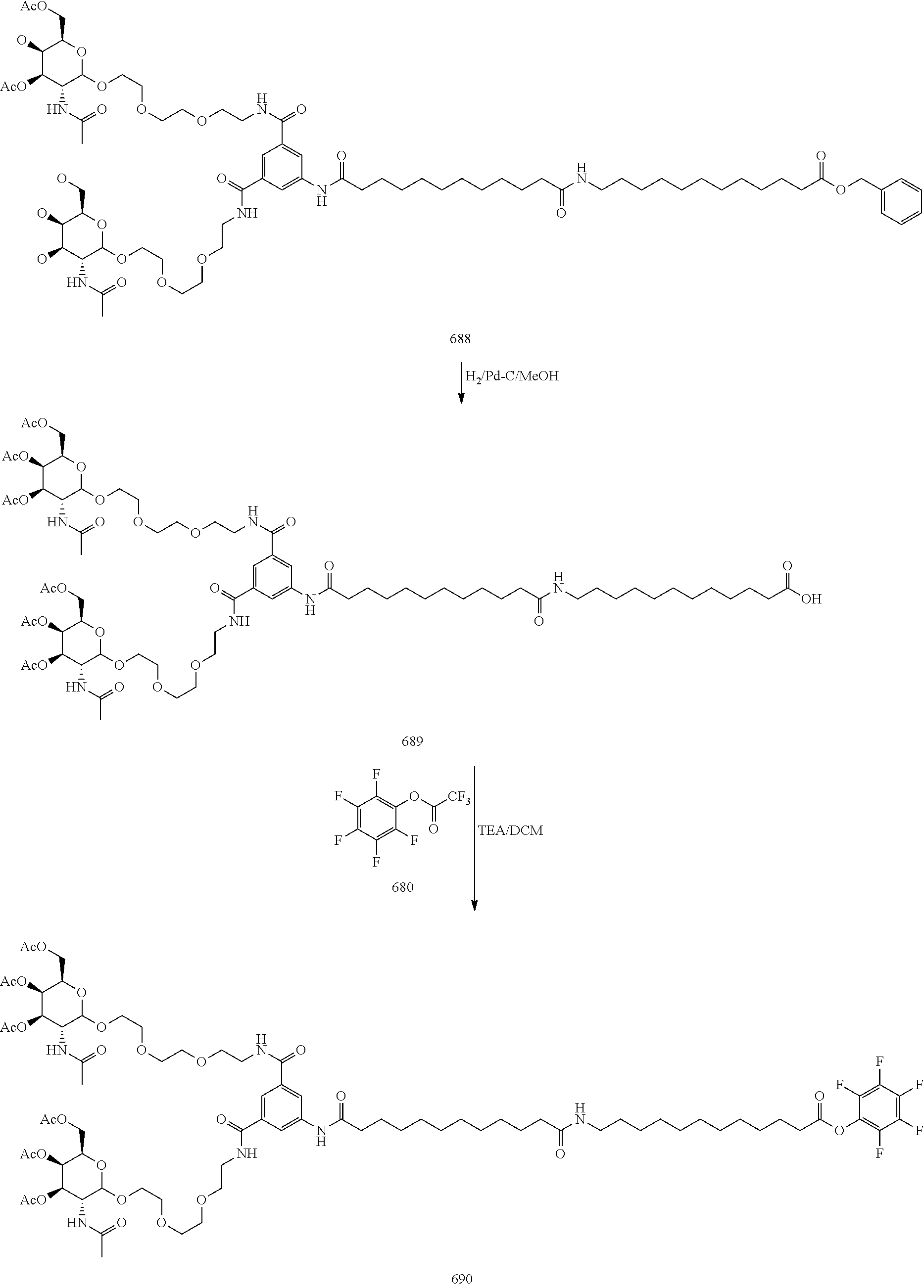

### Step 1. Preparation of 12-((tert-butoxycarbonyl)amino)dodecanoic acid 684

A solution of 12-aminododecanoic acid (682) (5.0 g, 23.3 mmol), di-tert-butyl decarbonate (683) (6.1 g, 27.9 mmol) and triethylamine (6.3 ml, 46.6 mmol) in methanol (75 ml) was heated to 60° C. for 3 h then at room temperature overnight. Upon completion, the solution was concentrated in vacuo to dryness and used in the next step without further purification.

### Step 2. Preparation of benzyl 12-((tert-butoxycarbonyl)amino)dodecanoate 685

A solution of crude 12-((tert-butoxycarbonyl)amino)dodecanoic acid (684) (9.0 g, 30.0 mmol), benzyl alcohol (685) (3.1 g, 30.0 mmol), EDC hydrochloride (6.9 g, 36.0 mmol) and triethylamine (12 ml, 90.0 mmol) in dichloromethane (100 ml) was stirred at room temperature overnight. Upon completion, the solution was washed with saturated sodium bicarbonate solution (100 ml) and brine (100 ml). The dichloromethane solution was dried on magnesium sulfate, filtered and concentrated to dryness. Purification by column chromatography on silica gel 60 (Gradient: 0 to 50% ethyl acetate in hexanes) afforded the title compound as a colorless solid (2.0 g, 21% over two steps).

### Step 3. Preparation of 12-(benzyloxy)-12-oxododecan-1-aminium trifluoroacetate 687

A solution of benzyl 12-((tert-butoxycarbonyl)amino)dodecanoate (686) (2.0 g, 4.9 mmol), dichloromethane (15 ml) and TFA (5 ml) was stirred overnight at room temperature. The reaction mixture was concentrated to dryness to afford the product as a viscous oil (2.1 g. quantitative).

### Step 4. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy) ethyl)carbamoyl)-5-(12-((12-(benzyloxy)-12-oxododecyl)amino)-12-oxododecanamido) benzamido)ethoxy)ethoxy)ethoxy)-2-(acetoxymethyl)tetrahydro-2H-pyran-3,4-diyl diacetate 688

A solution of 12-((3-((2-(2-(2-(((3R,4R,5R,6R)-3-acetamido-4,5-diacetoxy-6-(acetoxymethyl) tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)ethoxy)-ethyl) carbamoyl)phenyl)amino)-12-oxododecanoic acid (688) (750 mg, 0.54 mmol), 12-(benzyloxy)-12-oxododecan-1-aminium trifluoroacetate (687) (225 mg, 0.54 mmol), HBTU (210 mg, 0.54 mmol) and diisopropylethylamine (0.3 ml, 1.62 mmol) in dichloromethane (30 ml) was stirred overnight at room temperature. The solution was diluted with dichloromethane (50 ml) and washed with saturated bicarbonate solution (100 ml). The dichloromethane was dried on magnesium sulfate, filtered and concentrated in vacuo to dryness. The residue was purified by column chromatography on silica gel 60 (gradient: 0 to 10% methanol in dichloromethane) to afford the title compound (688) as a colorless solid (605 mg, 70%).

### Step 5. Preparation of 12-(12-((3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)ethoxy)ethyl)carbamoyl)phenyl)amino)-12-oxododecanamido)dodecanoic acid 689

Hydrogenation was conducted as previously described to give (689) (350 mg, 55%)

### Step 6. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)-ethoxy)ethoxy) ethyl)carbamoyl)-5-(12-oxo-12-((12-oxo-12-(perfluorophenoxy)-dodecyl)amino) dodecanamido)benzamido)ethoxy)ethoxy)ethoxy)-2-(acetoxymethyl)-tetrahydro-2H-pyran-3,4-diyl diacetate 690

PFP ester formation was conducted as described previously to give the required product (690) (112 mg, 23%). ¹H NMR (400 MHz, DMSO-d₆) δ 10.12 (s, 1H), 8.91 (s, 1H), 8.65 (t, J=5.5 Hz, 1H), 8.52 (t, J=5.6 Hz, 1H), 8.23 (d, J=1.5 Hz, 1H), 8.14 (t, J=1.4 Hz, 2H), 7.91 (d, J=1.6 Hz, 1H), 7.80 (d, J=9.2 Hz, 2H), 7.68 (t, J=5.6 Hz, 1H), 5.21 (d, J=3.4 Hz, 2H), 4.97 (dd, J=11.2, 3.4 Hz, 2H), 4.54 (d, J=8.5 Hz, 2H), 4.07-3.96 (m, 6H), 3.88 (dt, J=11.2, 8.9 Hz, 2H), 3.81-3.74 (m, 2H), 3.64-3.36 (m, 24H), 3.15-3.03 (m, 6H), 2.99 (q, J=6.5 Hz, 2H), 2.76 (t, J=7.2 Hz, 1H), 2.31 (t, J=7.4 Hz, 1H), 2.10 (s, 6H), 1.99 (s, 7H), 1.89 (s, 7H), 1.76 (s, 6H), 1.70-1.53 (m, 3H), 1.47 (q, J=7.1 Hz, 2H), 1.40-1.10 (m, 29H). Mass (ESI+) m/z 839.7 (M+2H).

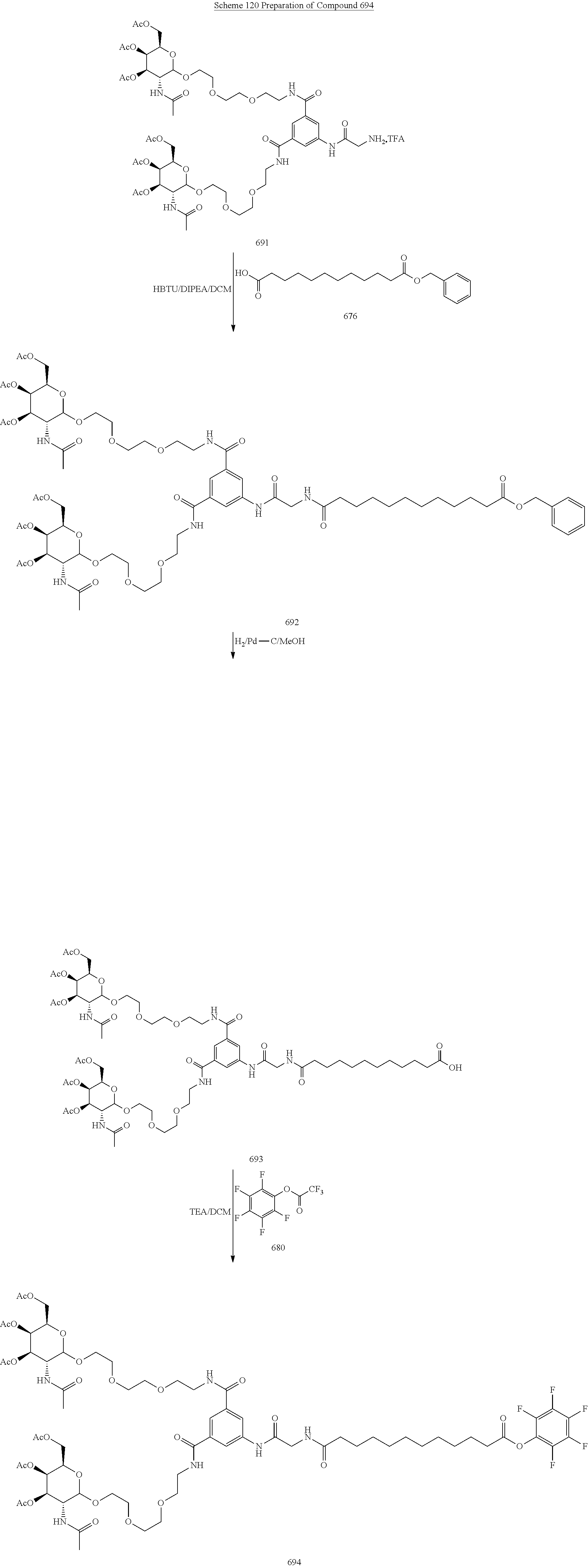

### Step 1. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy) ethyl)carbamoyl)-5-(2-(12-(benzyloxy)-12-oxododecanamido)acetamido)benzamido)ethoxy) ethoxy)ethoxy)-2-(acetoxymethyl)tetrahydro-2H-pyran-3,4-diyl diacetate 692

A solution of 2-((3-((2-(2-(2-(((3R,4R,5R,6R)-3-acetamido-4,5-diacetoxy-6-(acetoxymethyl) tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)-ethoxy)ethyl) carbamoyl)phenyl)amino)-2-oxoethan-1-aminium trifluoroacetate (691) (1.0 g, 0.8 mmol), 12-(benzyloxy)-12-oxododecanoic acid (676) (256 mg, 0.8 mmol), HBTU (341 mg, 0.9 mmol) and diisopropylethylamine (0.4 ml, 2.4 mmol) in dichloromethane (20 ml) was stirred overnight at room temperature. Upon completion, the reaction mixture was diluted with dichloromethane (80 ml) and washed with saturated sodium bicarbonate (100 ml). The solution was dried on magnesium sulfate, filtered and concentrated in vacuo to dryness. The residue was purified by column chromatography on silica gel 60 (gradient: 0 to 10% methanol in dichloromethane) to afford the title compound as a colorless solid (0.8 g, 68%).

### Step 2. Preparation of 12-((2-((3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethyl)carbamoyl)-5-((2-(2-(2-(((3S,4S,5S,6S)-3-acetamido-4,5-diacetoxy-6-(acetoxymethyl)tetrahydro-2H-pyran-2-yl)oxy)ethoxy)ethoxy)ethyl)carbamoyl)phenyl)amino)-2-oxoethyl)amino)-12-oxododecanoic acid 693

Compound 693 was prepared using conditions similar to those described herein for a similar conversion (450 mg, 60%).

### Step 3. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)-ethoxy)ethyl)carbamoyl)-5-(2-(12-oxo-12-(perfluorophenoxy)dodecanamido)acetamido) benzamido)ethoxy)ethoxy)ethoxy)-2-(acetoxymethyl)tetrahydro-2H-pyran-3,4-diyl diacetate 694

Compound 694 was prepared using conditions similar to those described herein for a similar conversion (460 mg, 91%). Mass (ESI+) m/z 1537.8 (M+H).

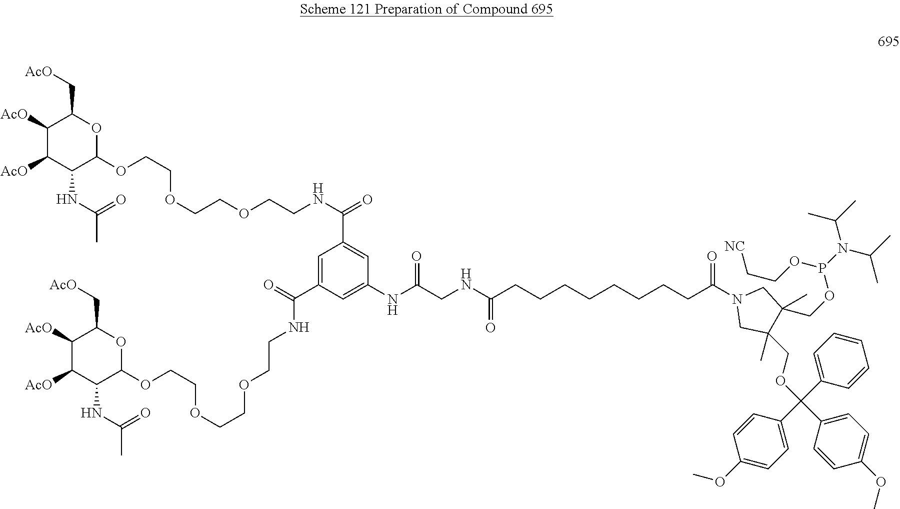 [SMILES: COc1ccc(C(OCC2(C)CN(C(=O)CCCCCCCCC(=O)NCC(=O)Nc3cc(C(=O)NCCOCCOCCOC4O[C@H](COC(C)=O)[C@H](OC(C)=O)[C@H](OC(C)=O)[C@H]4NC(C)=O)cc(C(=O)NCCOCCOCCOC4O[C@H](COC(C)=O)[C@H](OC(C)=O)[C@H](OC(C)=O)[C@H]4NC(C)=O)c3)CC2(C)COP(OCCC#N)N(C(C)C)C(C)C)(c2ccccc2)c2ccc(OC)cc2)cc1]

### Synthesis of (2R,2′R,3R,3′R,4R,4′R,5R,5′R)-((((((((5-(2-(10-(3-((bis(4-methoxyphenyl) (phenyl)methoxy)methyl)-4-((((2-cyanoethoxy)(diisopropylamino)phosphaneyl)-oxy)methyl)-3,4-dimethylpyrrolidin-1-yl)-10-oxodecanamido)acetamido)-isophthaloyl)bis(azanediyl))bis (ethane-2,1-diyl))bis(oxy))bis(ethane-2,1-diyl))bis(oxy))-bis(ethane-2,1-diyl))bis(oxy))bis(5-acetamido-2-(acetoxymethyl)tetrahydro-2H-pyran-6,3,4-triyl) tetraacetate 695

To a solution of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy)-ethyl) carbamoyl)-5-(2-(10-(3-((bis(4-methoxyphenyl)(phenyl)methoxy)methyl)-4-(hydroxymethyl)-3,4-dimethylpyrrolidin-1-yl)-10-oxodecanamido)acetamido)benzamido)-ethoxy)ethoxy)ethoxy)-2-(acetoxymethyl)tetrahydro-2H-pyran-3,4-diyl diacetate (672) (1.6 g, 0.9 mmol) and diisopropylethylamine (0.4 ml, 1.8 mmol) in anhydrous dichloromethane (25 ml) was added 2-Cyanoethyl N,N-diisopropylchlorophosphoramidite (0.3 ml, 1.35 mmol). The solution was stirred for 75 minutes at room temperature then concentrated to dryness. The residue was purified by column chromatography (gradient: 0 to 10% MeOH in DCM (0.1% TEA)) to afford the product as a colorless solid (1.1 g, 62%). 31P NMR (400 MHz, DMSO-d6): δ 146.76 (s), 146.42 (s, 2 overlapping signals), 146.34 (s). 1H NMR (400 MHz, DMSO-d6) δ 10.20 (s, 1H), 8.54 (t, J=5.6 Hz, 2H), 8.17-8.09 (m, 3H), 7.94 (s, 1H), 7.80 (d, J=9.2 Hz, 2H), 7.39-7.26 (m, 4H), 7.26-7.17 (m, 6H), 6.91-6.83 (m, 4H), 5.21 (d, J=3.4 Hz, 2H), 4.97 (dd, J=11.2, 3.4 Hz, 2H), 4.54 (d, J=8.5 Hz, 2H), 4.02 (s, 6H), 3.93-3.82 (m, 4H), 3.73 (s, 10H), 3.66-3.36 (m, 35H), 3.28-3.06 (m, 6H), 3.06-2.87 (m, 3H), 2.72-2.63 (m, J=11.5, 5.8 Hz, 2H), 2.10 (m, 12H), 1.99 (s, 6H), 1.89 (s, 6H), 1.77 (s, 6H), 1.47 (d, J=7.2 Hz, 4H), 1.23 (dq, J=13.9, 6.4 Hz, 18H), 1.17-1.04 (m, 10H), 0.98 (dt, J=13.4, 5.9 Hz, 10H).

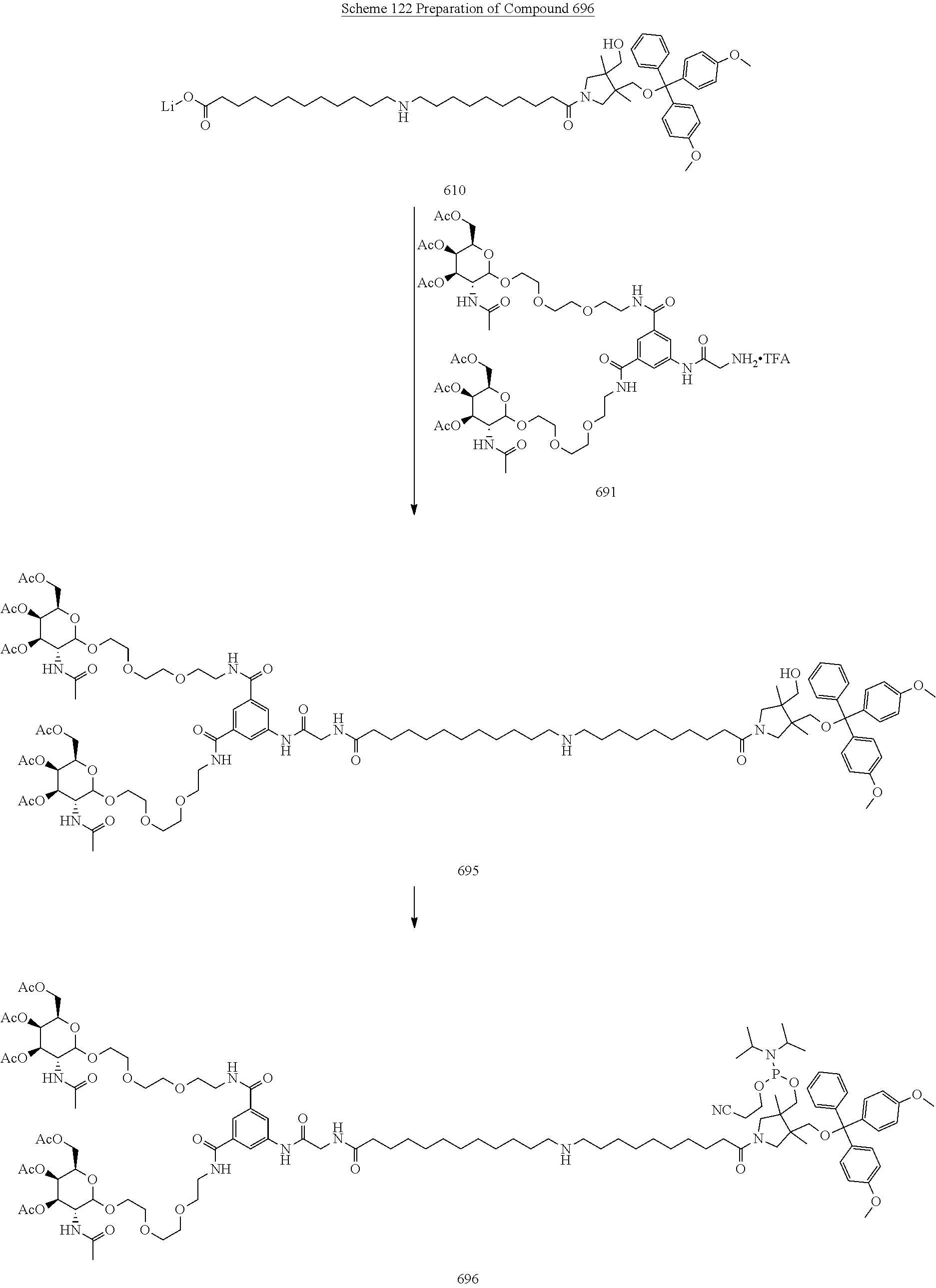

### Step 1. Preparation of (2S,3S,4S,5S)-5-acetamido-6-(2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)ethoxy) ethyl)carbamoyl)-5-(2-(12-((10-(3-((bis(4-methoxyphenyl)(phenyl)methoxy)methyl)-4-(hydroxymethyl)-3,4-dimethylpyrrolidin-1-yl)-10-oxodecyl)amino)dodecanamido) acetamido)benzamido)ethoxy)ethoxy)ethoxy)-2-(acetoxymethyl)tetrahydro-2H-pyran-3,4-diyl diacetate 6 95

Compound 695 was prepared using conditions similar to those described herein for a similar conversion (1.9 g, 61%).

### Step 2: Preparation of (2S,3S,4S,5S)-5-acetamido-6-2-(2-(2-(3-((2-(2-(2-(((3R,4R,5R,6R)-3-acetamido-4,5-diacetoxy-6-(acetoxymethyl)tetrahydro-2H-pyran-2-yl)oxy)ethoxy)-ethoxy) ethyl)carbamoyl)-5-(2-(12-((10-(3-((bis(4-methoxyphenyl)(phenyl)methoxy)-methyl-4-((((2-cyanoethoxy)(diisopropylamino)phosphaneyl)oxy)methyl)-3,4-dimethylpyrrolidin-1-yl)-10-oxodecyl)amino)dodecanamido)acetamido)benzamido)-ethoxy)ethoxy)ethoxy)-2-(acetoxymethyl)tetrahydro-2H-pyran-3,4-diyl diacetate 696

Compound 96 was prepared using conditions similar to those described herein for a similar conversion (1.35 g, 65%). ³¹P NMR (400 MHz, DMSO-d₆): δ 146.79 (s), 146.76 (s), 146.42 (s), 146.36 (s). ¹H NMR (400 MHz, DMSO-d₆) δ 10.19 (s, 1H), 8.54 (t, J=5.6 Hz, 2H), 8.13 (dd, J=6.1, 3.5 Hz, 3H), 7.94 (s, 1H), 7.80 (d, J=9.2 Hz, 2H), 7.71-7.65 (m, 1H), 7.39-7.25 (m, 4H), 7.25-7.17 (m, 4H), 6.92-6.83 (m, 4H), 5.21 (d, J=3.4 Hz, 2H), 4.97 (dd, J=11.2, 3.4 Hz, 2H), 4.54 (d, J=8.5 Hz, 2H), 4.07-3.97 (m, 6H), 3.94-3.82 (m, 4H), 3.82-3.74 (m, 2H), 3.73 (s, 6H), 3.62-3.45 (m, 23H), 3.42 (m, 6H), 3.27-2.92 (m, 14H), 2.73-2.62 (m, 2H), 2.10 (s, 8H), 1.99 (s, 9H), 1.89 (s, 6H), 1.77 (s, 6H), 1.52-1.42 (m, 6H), 1.22 (d, J=8.0 Hz, 24H), 1.17 (t, J=7.3 Hz, 11H), 1.09 (dt, J=6.7, 3.3 Hz, 9H), 1.03-0.92 (m, 9H).

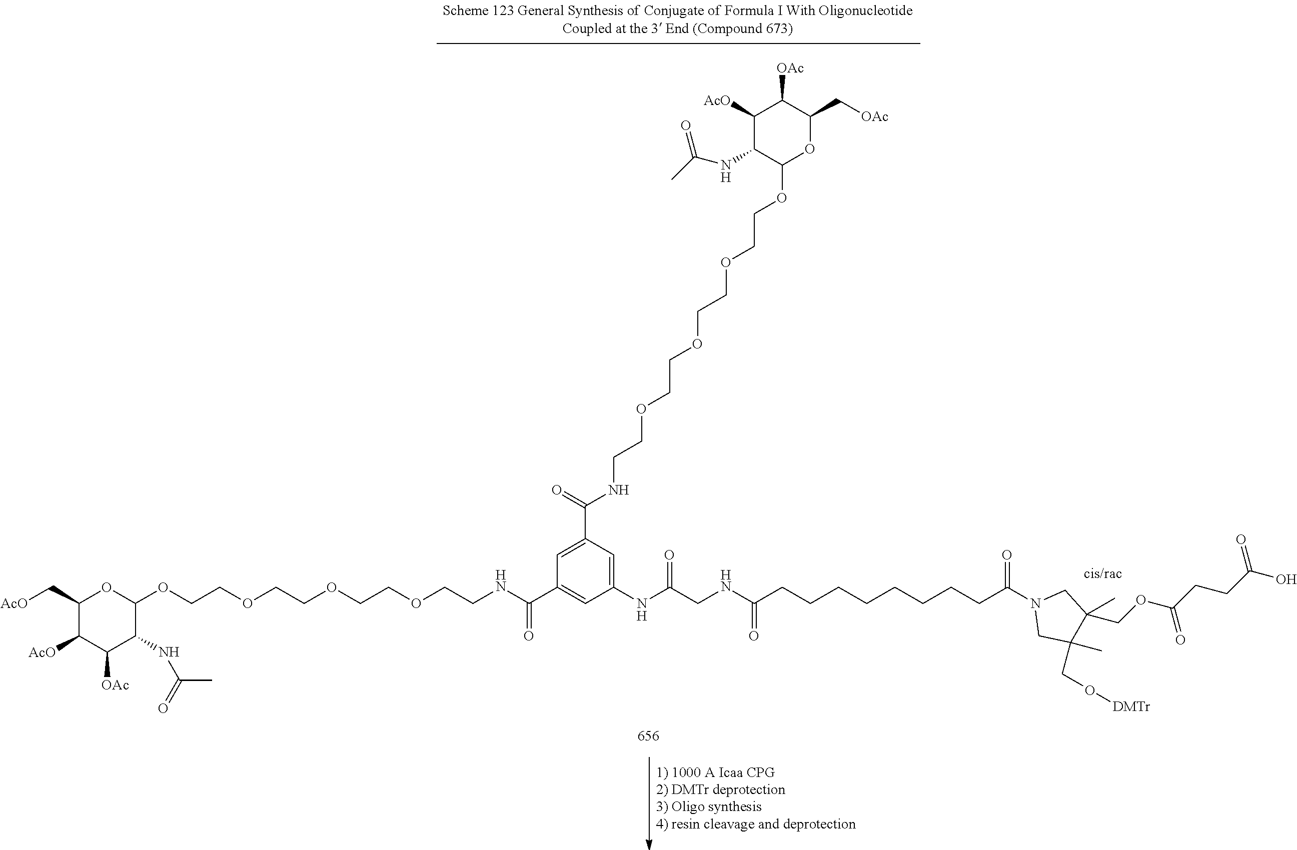

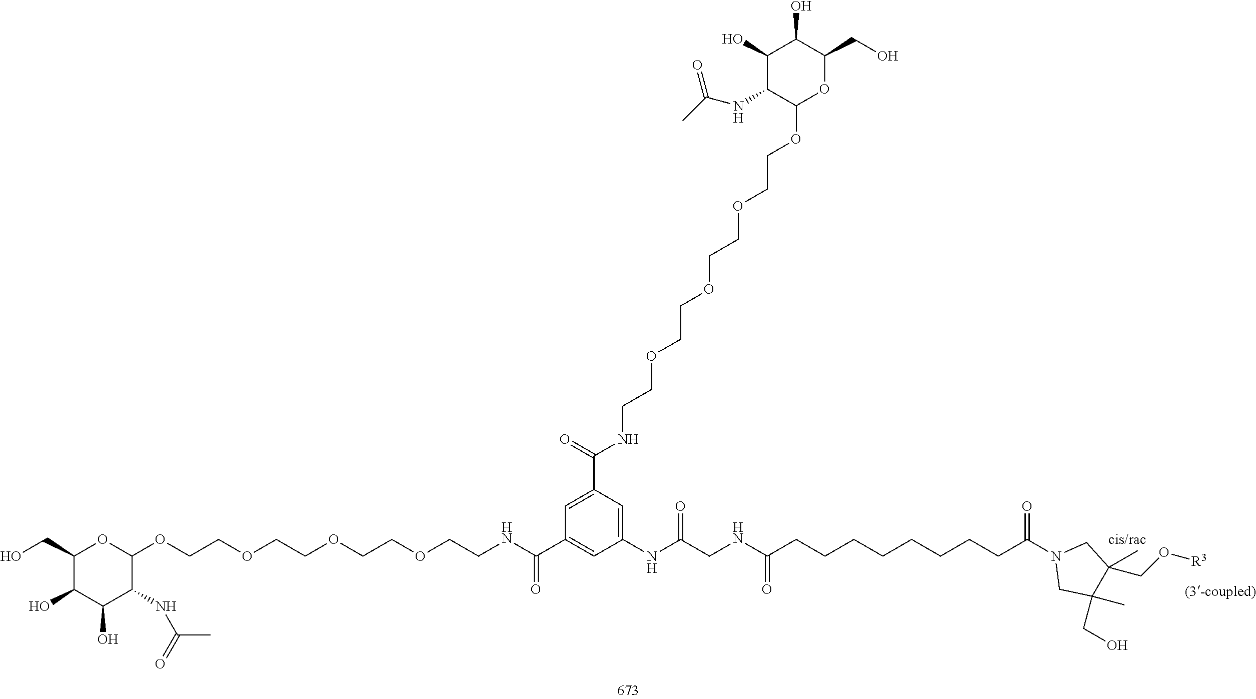

### General Method for Synthesizing Bidentate ASGPr Targeting Ligands from Succinate Ligands Exemplified for 4-((1-(10-((2-((3,5-bis((2-(2-(2-(2-(((3R,4R,5R,6R)-3-acetamido-4,5-diacetoxy-6-(acetoxymethyl)tetrahydro-2H-pyran-2-yl)oxy)ethoxy)ethoxy)ethoxy)-ethyl) carbamoyl)phenyl)amino)-2-oxoethyl)amino)-10-oxodecanoyl)-4-((bis(4-methoxyphenyl) (phenyl)methoxy)methyl)-3,4-dimethylpyrrolidin-3-yl)methoxy)-4-oxobutanoic acid 673

The succinate was loaded onto 1000 Å LCAA (long chain aminoalkyl) CPG (control pore glass) using standard amide coupling chemistry. LCAA CPG (2.0 g) was suspended in DCM (5 ml) and MeCN (7.6 ml). Diisopropylcarbodiimide (100 μl), N-hydroxy succinimide (110 μl, 30 μM/g), pyridine (110 μL) and 656 (200 mg, 0.1 mmol) were added and the suspension was gently mixed for 16 h at RT. The CPG was recovered by filtration, washed with DCM (×3) and MeCN (×3) and dried under high vacuum. A solution of 5% acetic anhydride/5% N-methylimidazole/5% pyridine in THF was added and the suspension agitated at RT for 2 h. The CPG was recovered by filtration, washed with DCM (×3) and MeCN (×3) and dried under high vacuum. Loading was determined to be 31.3 μmol/g (DMTr assay by UV/Vis 504 nm). The resulting GalNAc loaded CPG solid support was employed in automated oligonucleotide synthesis using standard procedures. Nucleotide deprotection followed by removal from the solid support (with concurrent galactosamine acetate deprotection) afforded the GalNAc-oligonucleotide conjugate 673.

### Examples 27a-27i

Using the general procedure illustrated in Scheme 123, the following conjugates (27a-27i) were prepared, wherein R³ is the modified TTR siRNA described in Table A below.

### Example 27a

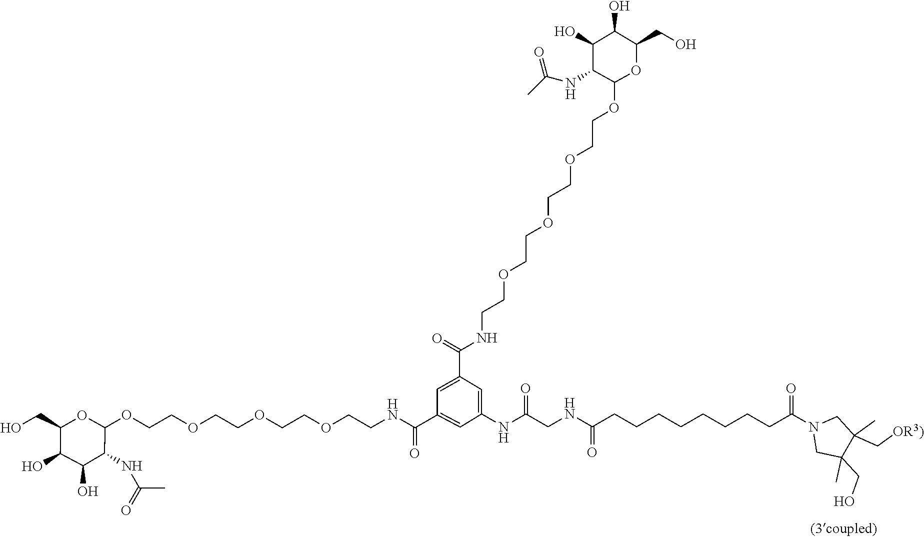

MS (+VE) calculated: 8184.7; measured: 8184.2

### Example 27b

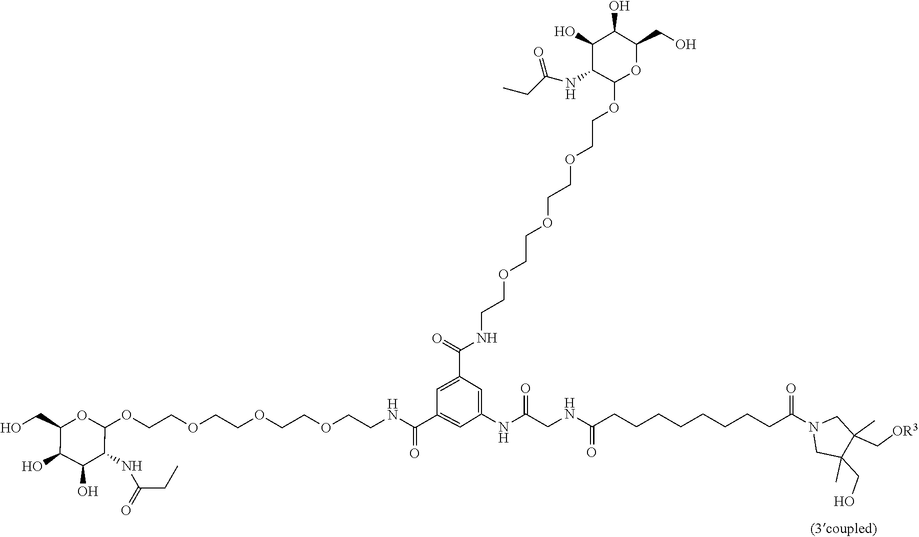

MS (+VE) calculated: 8212.7; measured: 8211.9

### Example 27c

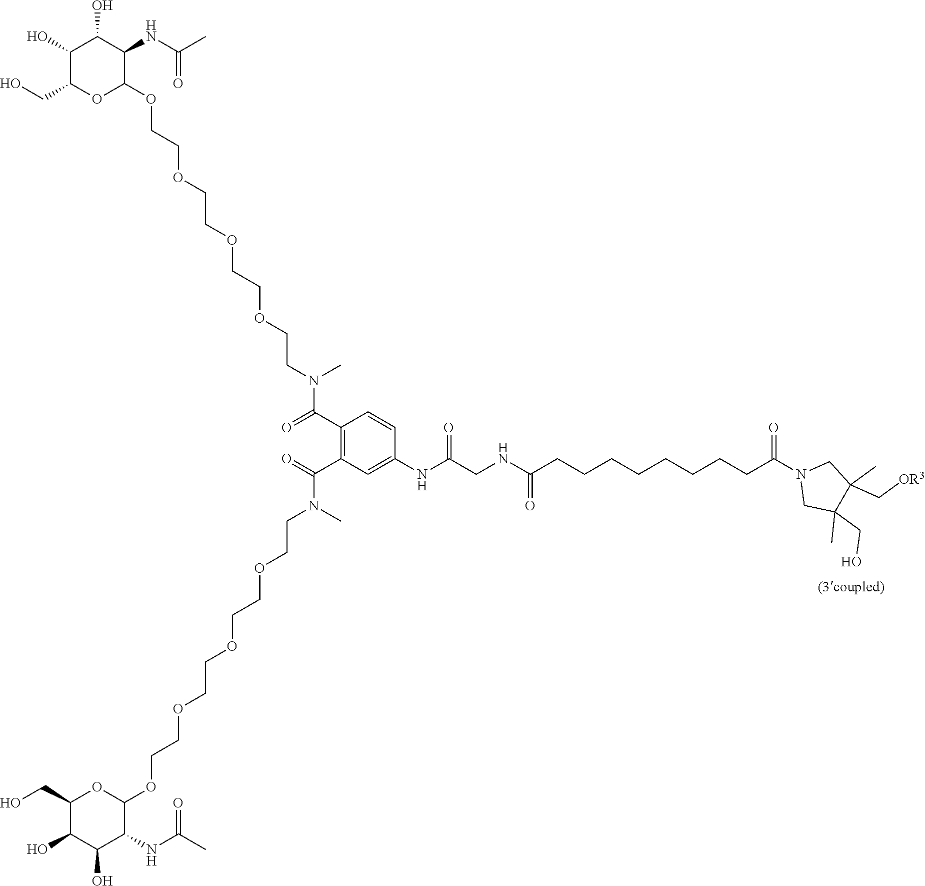

MS (+VE) calculated: 8212.7; measured: 8212.8

### Example 27d

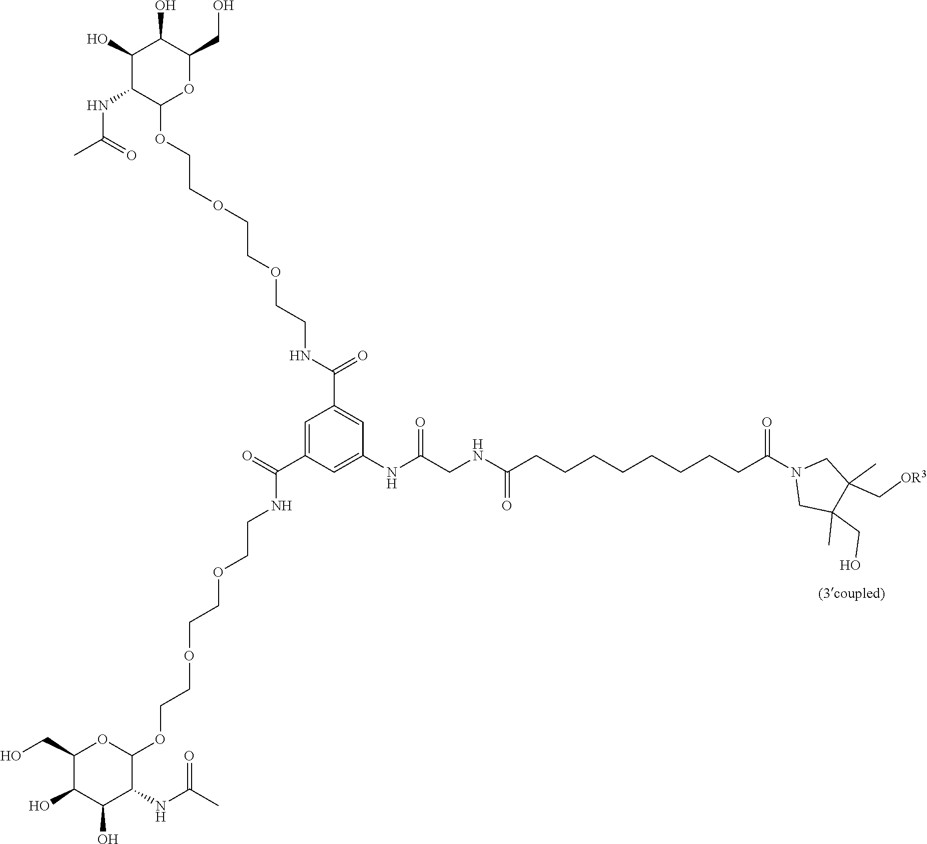

MS (+VE) calculated: 8096.6; measured: 8097.0

### Example 27e

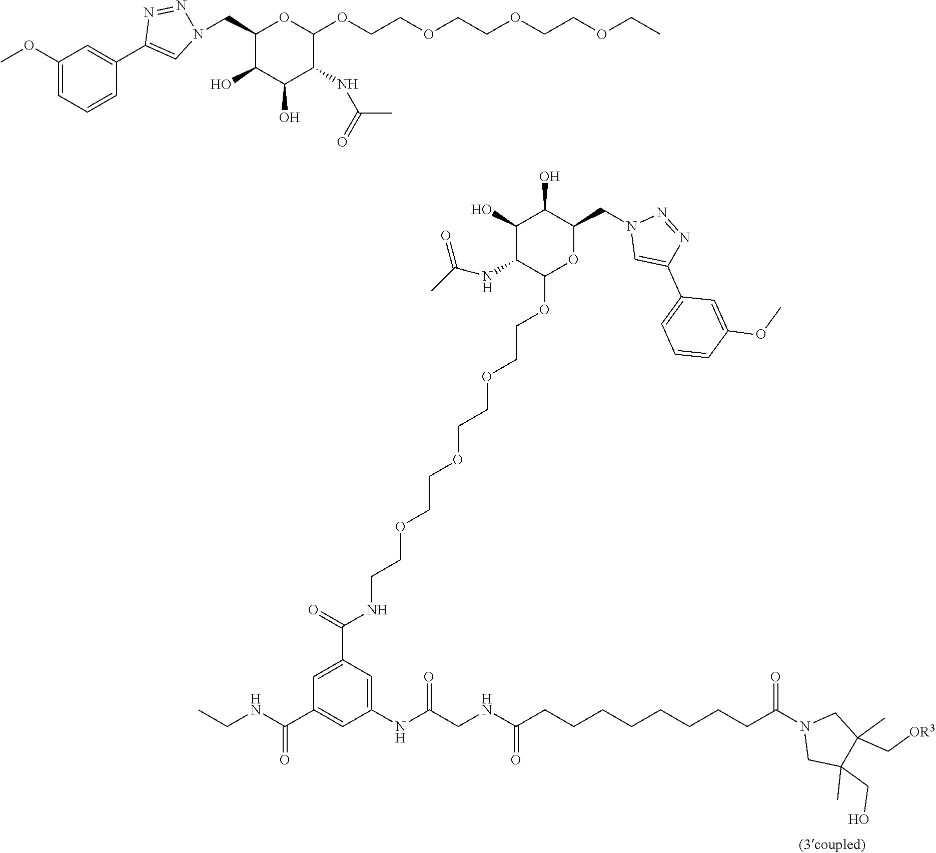

MS (+VE) calculated: 8499.0; measured: 8498.7

### Example 27f

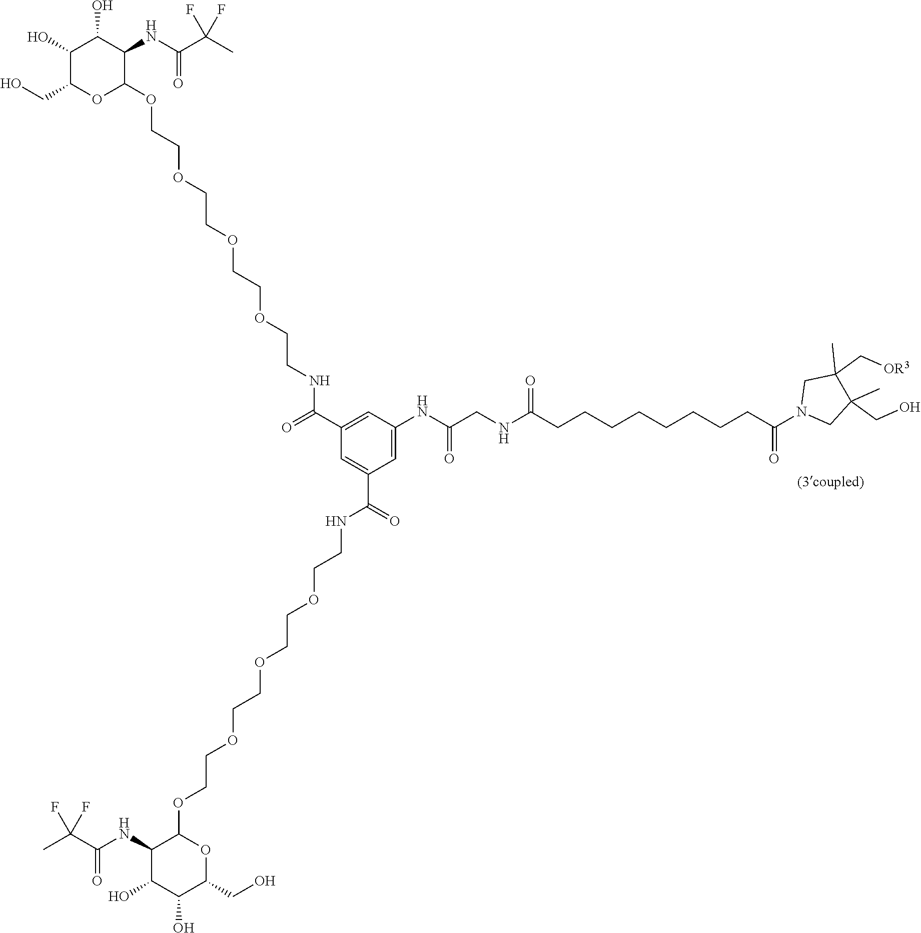

MS (+VE) calculated: 8284.7; measured: 8283.8

### Example 27 g

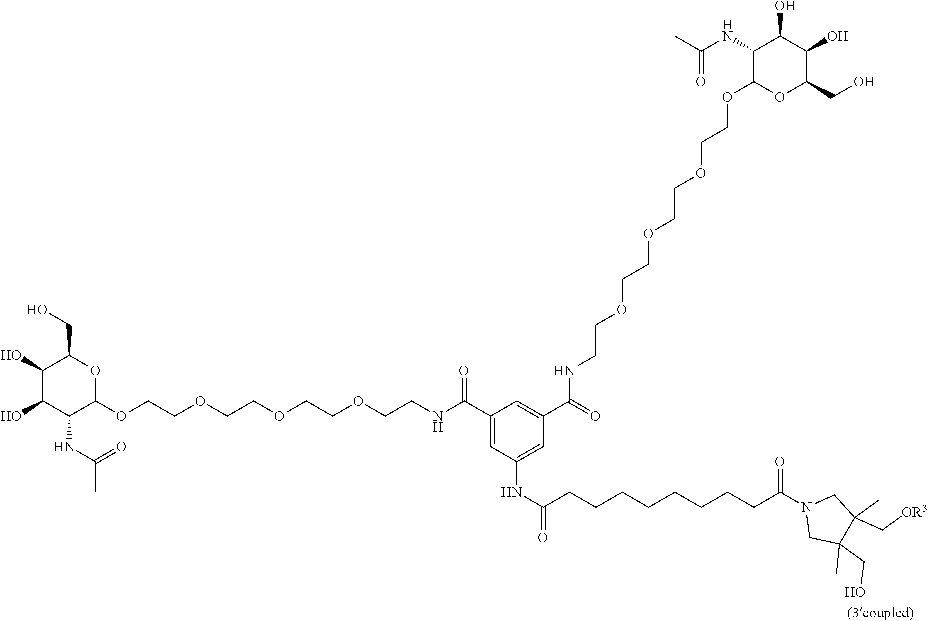

MS (+VE) calculated: 7596.0; measured: 7596.8

### Example 27h

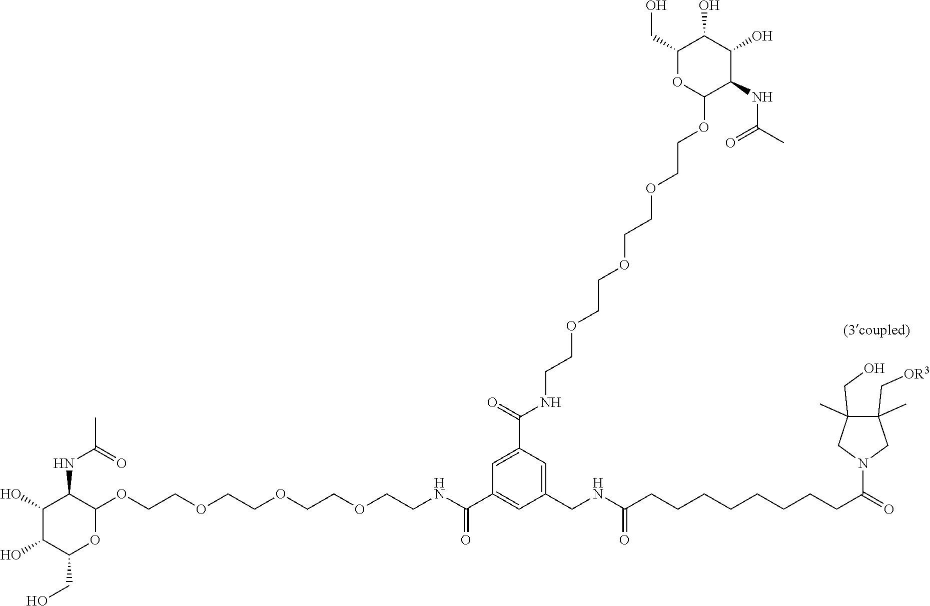

### Example 27i

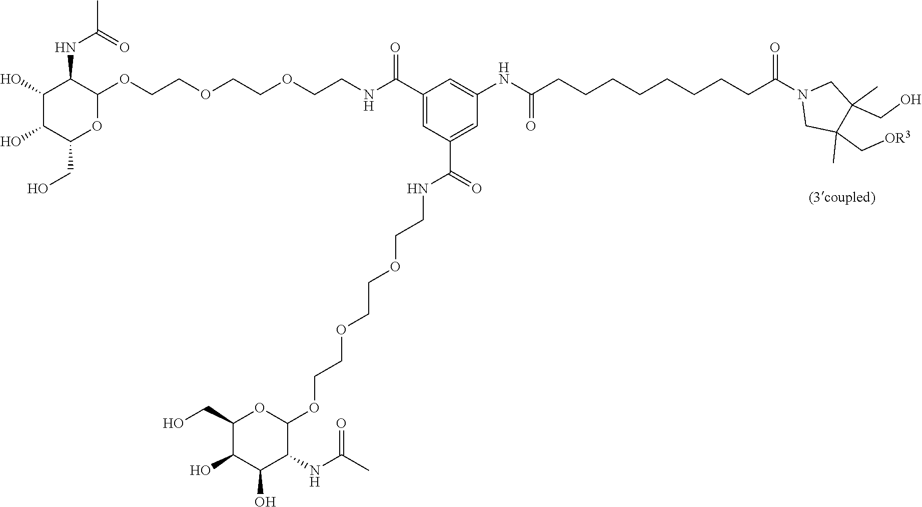

### Schemes 124 and 125 General Synthesis of Conjugates of Formula I with Oligonucleotide Coupled at the 5′ End (Compound 698)

Pentafluorophenyl esters were coupled to a C₆ 5′-amino modifier with phosphate/phosphorothioate linkage on the sense strand oligonucleotide using standard coupling conditions. Standard cleavage and deprotection afforded the desired sense strand conjugate. For example the pentafluorophenyl ester 681 was used to afford the conjugate 698 below (Scheme 124).

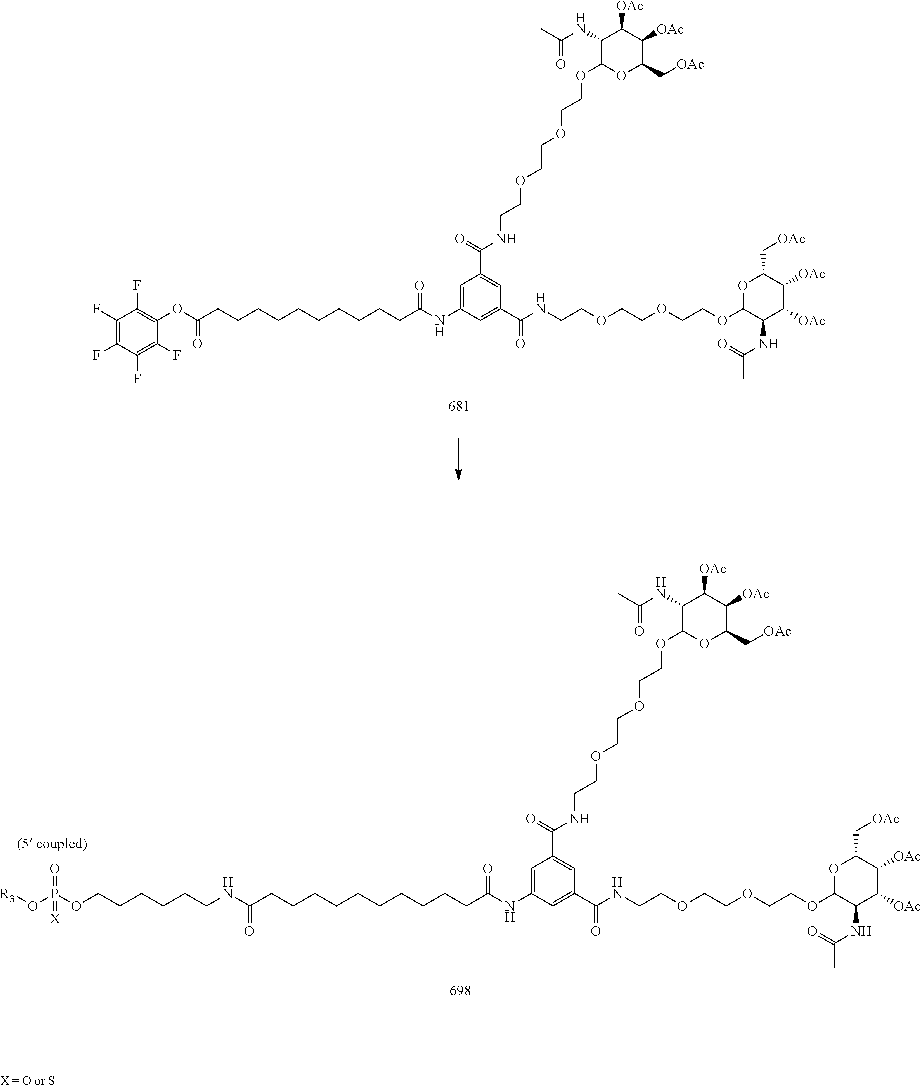

Phosphoramidites were coupled to the 5′ hydroxyl of the sense strand terminal nucleotide using standard phosphoramidite coupling chemistry. Standard cleavage and deprotection afforded the desired sense strand conjugate. For example phosphoramidite 695 was used to afford the conjugate 699 below (Scheme 125).

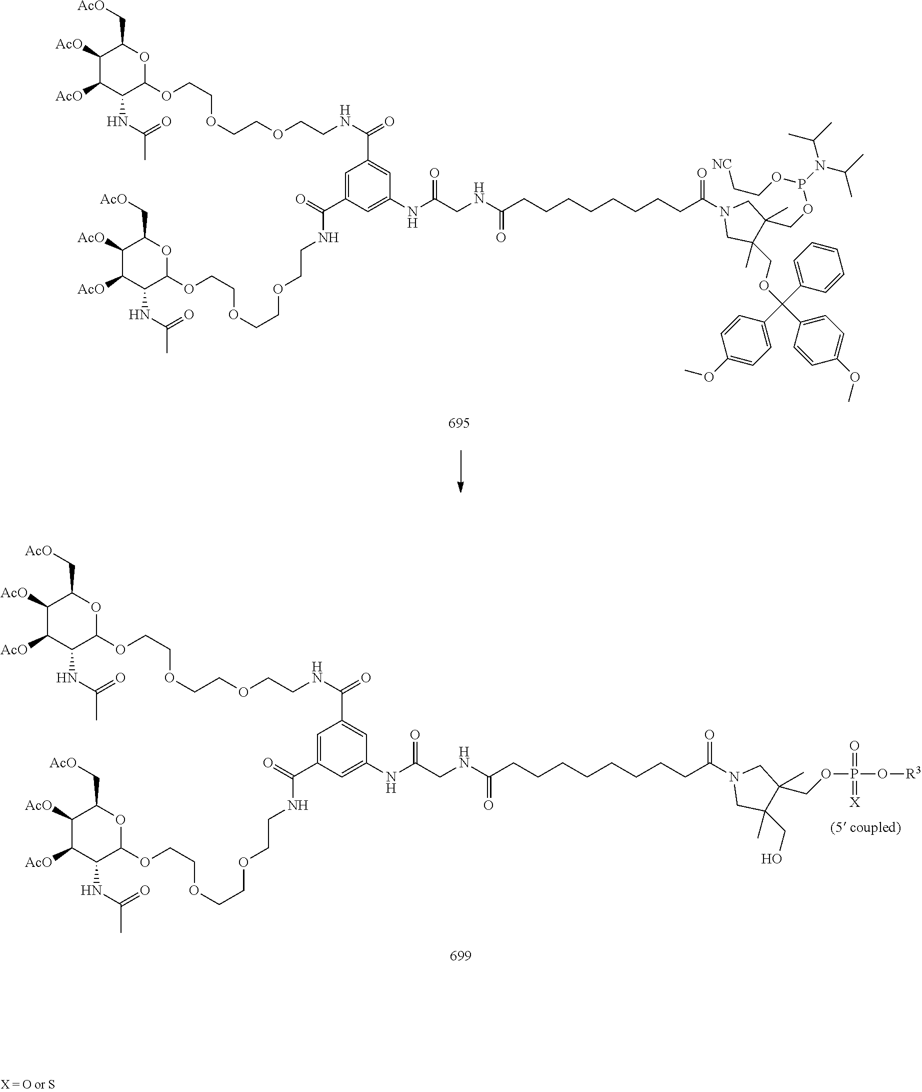

### Examples 27j-27k

Using the general procedure illustrated in Schemes 124 and 125, the following conjugates (27j-27k) were prepared, wherein R³ is the modified TTR siRNA described in Table A below.

### Example 27j

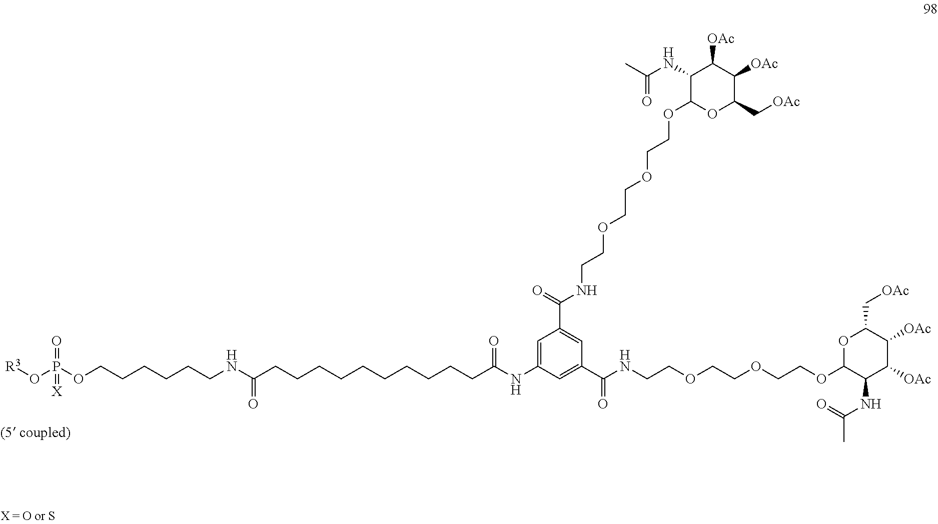

MS (+VE) calculated: 8056.7; measured: 8056.1

### Example 27k

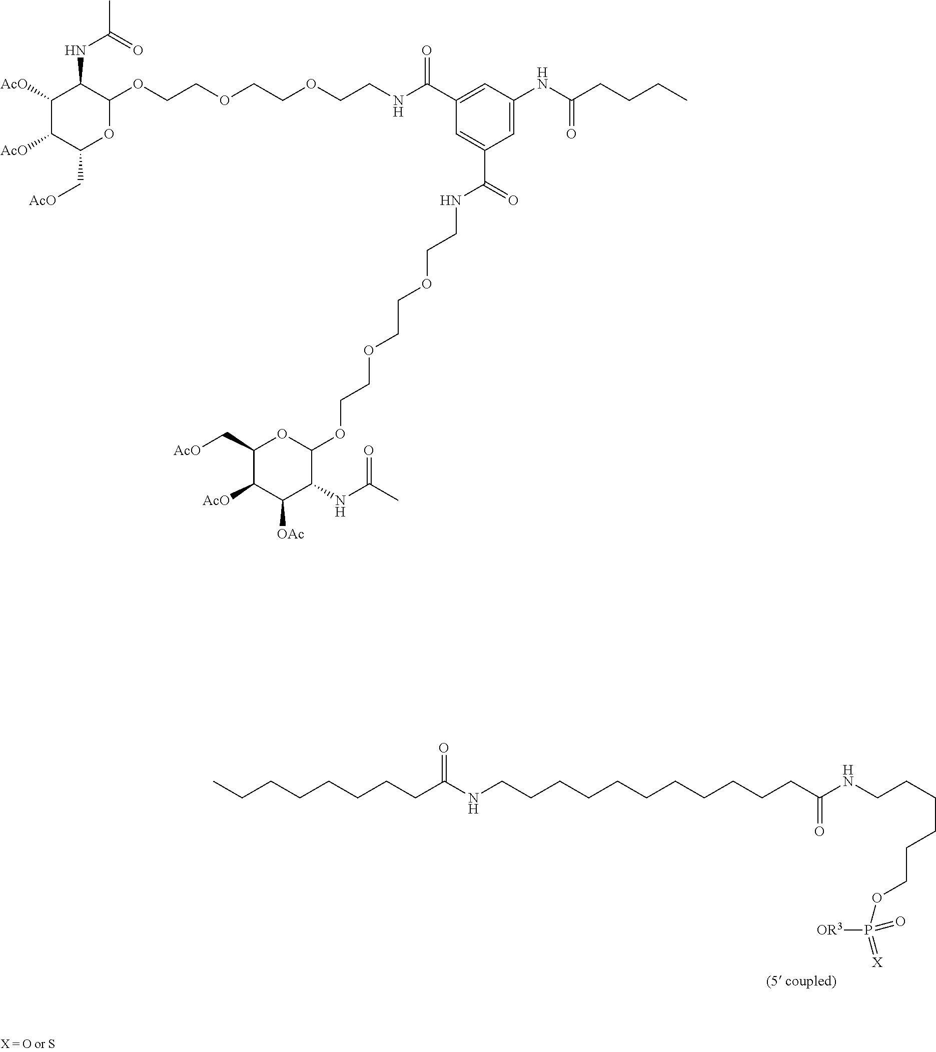

MS (+VE) calculated: 8254.0; measured: 8253.5

### Example 28 In Vivo Testing of TTR siRNA Conjugates Co-Delivered with Polymer Micelle

The conjugate of Example 27d wherein the R³ is the modified TTR siRNA described in Table 28-1 below (the Ligand) was tested for in vivo activity in a wild-type mouse model of TTR knockdown. The Ligand is a possible treatment for the orphan disease of TTR (Transthyretin) amyloidosis. The inclusion of a membrane-destabilizing polymer of formula:

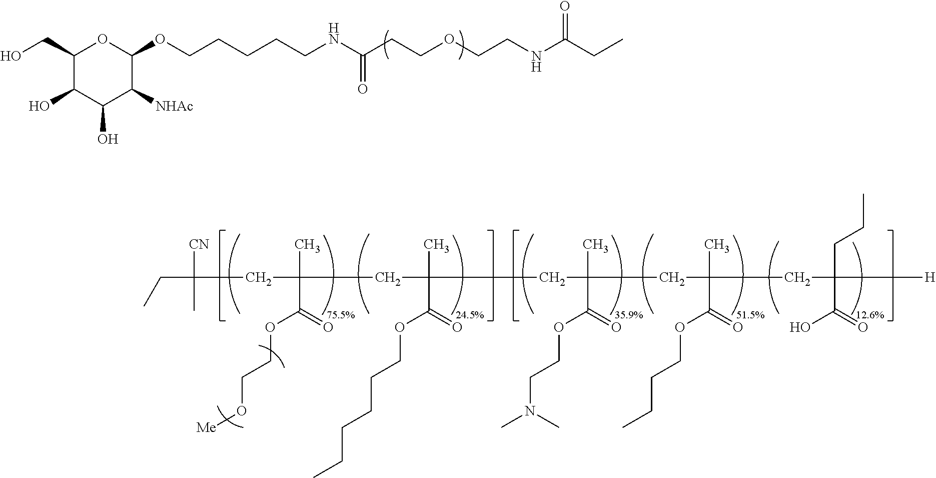

with the Ligand was found to enhance endosomal release of the conjugate following cellular uptake by hepatocytes. In those afflicted with TTR amyloidosis, the misfolding and aggregation of the Transthyretin protein is known to be associated with disease progression. By using the Ligand combined with the membrane-destabilizing polymer, the amount of misfolded/aggregated protein in the patient can be reduced with a possible result of halting the progression of the disease.

TABLE 28-1
Chemically Modified TTR siRNA duplexes

| Sense strand | Antisense strand |
|---|---|
| 5′-3′ | 5′-3′ |
| AsasCaGuGuUCUuGcUcUaUaA | usUsaUaGaGcAagaAcAcUgUususu |
| (SEQ ID NO:1) | (SEQ ID NO :2) |

2′-O-Methyl nucleotides=lower case; 2′-Fluoro nucleotides=UPPER CASE;
Phosphorothioate linker=s; Unmodified=UPPER CASE
Both the TTR siRNA sequence & animal model were described by Nair et al. J. Am. Chem. Soc., 2014, 136 (49), pp 16958-16961. All animal-related procedures were conducted according to written operating procedures, in accordance with Canadian Council on Animal Care (CCAC) Guidelines on Good Animal Practices and approved by the local Institutional Animal Care and Use Committee (IACUC).
Treatment: Three groups of female C57BL/6 mice (n=4) were administered a single 0.35 mg/kg dose of the Ligand combined with 10 mg/kg, 20 mg/kg or 30 mg/kg of the polymer once on Day 0 (1 dose per animal) via subcutaneous injection in the scapular region. As controls, two groups of animals were administered a 1.8 mg/kg or 0.35 mg/kg dose of Ligand only (no polymer). Animals administered vehicle only (PBS) served as the negative control.
Collections: All animals were bled at defined time points after test article administration (Days 2, 5, 7, 14 and 21) to determine maximum reductions in plasma TTR levels and the duration of pharmacologic activity.
Analysis: TTR protein levels in plasma samples were determined using the Abnova Prealbumin (Mouse) ELISA kit (Cedar Lane, catalogue number KA2070) as per the manufacturer's instructions. TTR plasma protein values were calculated for the individual plasma samples and the average of each group was determined. From these averages, the TTR protein levels relative to control (% relative to PBS treated animals) were determined.
Results: Experimental data are presented in Table 28-2. Values represent % TTR protein levels (relative to PBS Control) on Days 2, 5, 7, 14, 21, and 28 post treatment.
Conclusion: Animals treated with Ligand combined with as little as 10 mg/kg of polymer exhibited a marked increase in knockdown of target mRNA compared to Ligand alone. Furthermore, the onset of activity was more rapid in the presence of the polymer and the duration of effect was dramatically extended. Mice treated with polymer alone at the 30 mg/kg dose did not show a reduction in TTR protein relative to PBS.
Plasma TTR Protein Levels in Mice after Single Subcutaneous Administration of Ligand from Table 28-1, in the Presence or Absence of Various Polymer Amounts.
TTR protein data expressed as percent of PBS treated mouse values.

| Ligand | Polymer | | | | | | |
|---|---|---|---|---|---|---|---|
| Dose | Dose | Day | Day | Day | Day | Day | Day |
| (mg/kg) | (mg/kg) | 2 | 5 | 7 | 14 | 21 | 28 |
| 1.8 | 0 | 28.2 | 13.9 | 14.2 | 29.5 | 46.7 | 65.6 |
| 0.35 | 0 | 59.0 | 49.1 | 49.8 | 66.4 | 87.0 | 94.7 |
| 0.35 | 10 | 14.4 | 7.6 | 7.8 | 16.0 | 41.6 | 54.1 |
| 0.35 | 20 | 6.9 | 2.5 | 2.3 | 2.6 | 4.7 | 11.4 |
| 0.35 | 30 | 9.8 | 3.0 | 2.7 | 3.4 | 7.8 | 12.8 |
| 0 | 30 | 84.6 | 110.3 | 102.6 | 97.3 | 89.3 | 84.5 |

### Example 29. Dose Titration of the Ligand from Example 28 Co-Delivered Subcutaneously with a Membrane-Destabilizing Polymer

The Ligand from Example 28 was tested for in vivo activity in a wild-type mouse model of TTR knockdown. A polymer of formula:

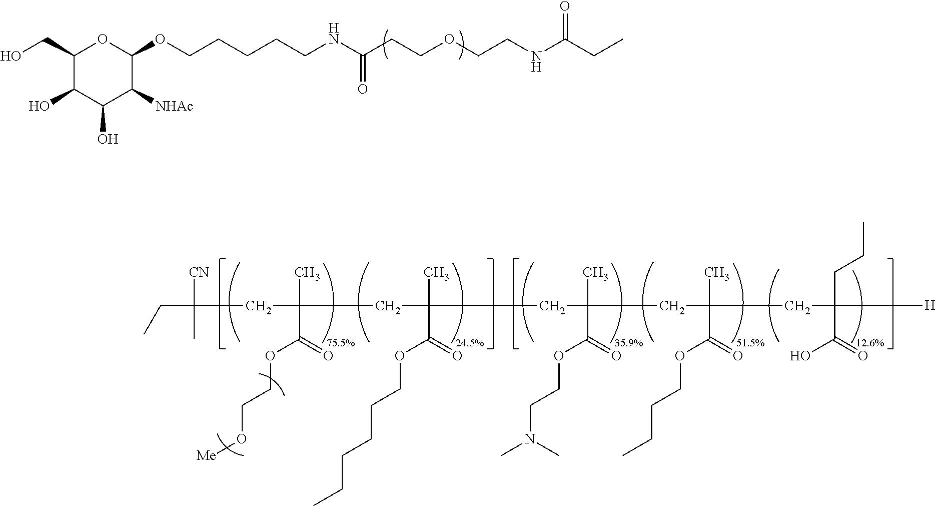

was co-delivered with the ligand.
Treatment: Female C57BL/6 mice (n=3) were treated as a single dose subcutaneously (scapular region) with either PBS, Ligand alone (dosed at 2.5 mg/kg, 0.50 mg/kg, and 0.05 mg/kg conjugate), and Ligand combined with polymer (conjugate dosed at 0.50 mg/kg or 0.05 mg/kg and polymer dosed at 0.3 mg/kg, 1 mg/kg, 3 mg/kg, 10 mg/kg, or 30 mg/kg).
Collections: All animals were bled at defined time points after test article administration (days 1, 2, 6, 9, 14 and 21) to determine maximum reductions in plasma TTR levels and the duration of pharmacologic activity.
Analysis: TTR protein levels in plasma samples were determined using the Abnova Prealbumin (Mouse) ELISA kit (Cedar Lane, catalogue number KA2070) as per the manufacturer's instructions. TTR plasma protein values were calculated for the individual plasma samples and the average of each group was determined. From these averages, the TTR protein levels relative to control (% relative to PBS treated animals) were determined.
Results: Experimental data are presented in Table 29-1. Values represent % TTR protein levels (relative to PBS Control) on Days 1, 2, 6, 9, 14 & 21 post treatment.
Conclusion: Animals treated with the Ligand combined with >10 mg/kg of the polymer exhibited a marked increase in knockdown of target mRNA relative to animals treated with Ligand only. Titration of the polymer demonstrated that a polymer dose of 10 mg/kg or greater enhanced endosomal release, especially at lower conjugate doses (e.g. 0.05 mg/kg). When the polymer dose is increased to 30 mg/kg, similar TTR knockdown was observed between the 0.05 mg/kg and 0.50 mg/kg conjugate doses. Rapid onset of activity and extended duration of effect were also observed.
Plasma TTR Protein Levels in Mice after Single Subcutaneous Administration of Ligand, in the Presence or Absence of Various Polymer Amounts.
TTR protein data expressed as percent of PBS treated mouse values.

| Ligand | Polymer | | | | | | |
|---|---|---|---|---|---|---|---|
| Dose | Dose | Day | Day | Day | Day | Day | Day |
| (mg/kg) | (mg/kg) | 1 | 2 | 6 | 9 | 14 | 21 |
| 2.5 | 0 | 52.1 | 8.8 | 5.1 | 6.8 | | |
| 0.05 | 0 | 102.6 | 74.8 | 90.8 | 99.0 | | |
| 0.05 | 0.3 | 106.2 | 100.8 | 93.0 | 92.5 | | |
| 0.05 | 1 | 93.5 | 92.6 | 73.5 | 92.3 | | |
| 0.05 | 3 | 103.7 | 78.9 | 82.3 | 94.3 | | |
| 0.05 | 10 | 60.8 | 12.8 | 26.0 | 30.6 | | |
| 0.05 | 30 | 25.3 | 3.3 | 2.0 | 2.4 | | |
| 0.50 | 0 | 77.1 | 41.3 | 32.5 | 44.6 | 56.2 | 79.7 |
| 0.50 | 0.3 | 92.9 | 30.9 | 29.2 | 38.8 | 51.1 | 79.4 |
| 0.50 | 1 | 77.5 | 33.2 | 26.7 | 41.1 | 43.0 | 67.3 |
| 0.50 | 3 | 70.3 | 16.6 | 18.6 | 28.0 | 38.9 | 65.0 |
| 0.50 | 10 | 30.5 | 3.1 | 2.4 | 5.1 | 3.0 | 16.8 |
| 0.50 | 30 | 26.3 | 3.2 | 2.0 | 2.4 | 1.7 | 1.8 |

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for delivering a nucleic acid to a cell comprising contacting the cell with, 1) a membrane-destabilizing polymer; and 2) a nucleic acid conjugate of Formula (X):
A-B-C (X)
wherein: A is a targeting ligand, B is an optional linker, and C is a nucleic acid;
wherein the membrane-destabilizing polymer is a polymer of formula (XX):
T⁵-L-[PEGMAₘ-M²ₙ]ᵥ[DMAEMA_{q}-PAAᵣ-BMAₛ]_{w} (XX)
wherein:
PEGMA is polyethyleneglycol methacrylate residue with 2-20 ethylene glycol units;
M² is a methacrylate residue selected from the group consisting of
a (C₄-C₁₈)alkyl-methacrylate residue;
a (C₄-C₁₈) branched alkyl-methacrylate residue;
a cholesteryl methacrylate residue;
a (C₄-C₁₈)alkyl-methacrylate residue substituted with one or more fluorine atoms; and
a (C₄-C₁₈) branched alkyl-methacrylate residue substituted with one or more fluorine atoms;
BMA is butyl methacrylate residue;
PAA is propyl acrylic acid residue;
DMAEMA is dimethylaminoethyl methacrylate residue;
m and n are each a mole fraction greater than 0, wherein m is greater than n and m+n=1;
q is a mole fraction of 0.2 to 0.75;
r is a mole fraction of 0.05 to 0.6;
s is a mole fraction of 0.2 to 0.75;
q+r+s=1;
v is 1 to 25 kDa;
w is 1 to 25 kDa;
T⁵ is a targeting moiety; and
L is absent or is a linking moiety.

2. A method for delivering a nucleic acid to the cytosol of a target cell within an animal, the method comprising: administering to the animal, (a) a membrane-destabilizing polymer, and (b) a nucleic acid conjugate of Formula (X):
A-B-C (X)
wherein A is a targeting ligand, B is an optional linker, and C is a nucleic acid, wherein the nucleic acid is delivered to the cytosol of the target cell;
wherein the membrane-destabilizing polymer is a polymer of formula (XX):
T⁵-L-[PEGMAₘ-M²ₙ]ᵥ[DMAEMA_{q}-PAAᵣ-BMAₛ]_{w} (XX)
wherein:
PEGMA is polyethyleneglycol methacrylate residue with 2-20 ethylene glycol units;
M² is a methacrylate residue selected from the group consisting of
a (C₄-C₁₈)alkyl-methacrylate residue;
a (C₄-C₁₈) branched alkyl-methacrylate residue;
a cholesteryl methacrylate residue;
a (C₄-C₁₈)alkyl-methacrylate residue substituted with one or more fluorine atoms; and
a (C₄-C₁₈) branched alkyl-methacrylate residue substituted with one or more fluorine atoms;
BMA is butyl methacrylate residue;
PAA is propyl acrylic acid residue;
DMAEMA is dimethylaminoethyl methacrylate residue;
m and n are each a mole fraction greater than 0, wherein m is greater than n and m+n=1;
q is a mole fraction of 0.2 to 0.75;
r is a mole fraction of 0.05 to 0.6;
s is a mole fraction of 0.2 to 0.75;
q+r+s=1;
v is 1 to 25 kDa;
w is 1 to 25 kDa;
T⁵ is a targeting moiety; and
L is absent or is a linking moiety.

3. A method comprising, administering to an animal, 1) a membrane-destabilizing polymer; and 2) a nucleic acid conjugate of Formula (X):
A-B-C (X)
wherein A is a targeting ligand, B is an optional linker, and C is a nucleic acid;
wherein the membrane-destabilizing polymer is a polymer of formula (XX):
T⁵-L-[PEGMAₘ-M²ₙ]ᵥ-[DMAEMA_{q}-PAAᵣ-BMAₛ]_{w} (XX)
wherein:
PEGMA is polyethyleneglycol methacrylate residue with 2-20 ethylene glycol units;
M² is a methacrylate residue selected from the group consisting of
a (C₄-C₁₈)alkyl-methacrylate residue;
a (C₄-C₁₈) branched alkyl-methacrylate residue;
a cholesteryl methacrylate residue;
a (C₄-C₁₈)alkyl-methacrylate residue substituted with one or more fluorine atoms; and
a (C₄-C₁₈) branched alkyl-methacrylate residue substituted with one or more fluorine atoms;
BMA is butyl methacrylate residue;
PAA is propyl acrylic acid residue;
DMAEMA is dimethylaminoethyl methacrylate residue;
m and n are each a mole fraction greater than 0, wherein m is greater than n and m+n=1;
q is a mole fraction of 0.2 to 0.75;
r is a mole fraction of 0.05 to 0.6;
s is a mole fraction of 0.2 to 0.75;
q+r+s=1;
v is 1 to 25 kDa;
w is 1 to 25 kDa;
T⁵ is a targeting moiety; and
L is absent or is a linking moiety.

4. The method of claim 1 or 2, wherein A is a targeting ligand that specifically binds to a molecule on the surface of the target cell.

5. The method of claim 1 or 2, wherein T⁵ specifically binds to a molecule on the surface of the target cell.

6. The method of claim 2 or 3, wherein the nucleic acid conjugate and the membrane-destabilizing polymer are administered separately.

7. The method of claim 2 or 3, wherein the membrane-destabilizing polymer is administered after administration of the nucleic acid conjugate.

8. The method of claim 2 or 3, wherein the nucleic acid conjugate and the membrane-destabilizing polymer are administered together within a single composition.

9. The method of claim 5, wherein the targeting ligand and T⁵ are different and either (i) specifically bind to the same cell surface molecule or (ii) specifically bind to a different cell surface molecule on the target cell.

10. The method of claim 5, wherein the targeting ligand and T⁵ are the same and each specifically binds to the same cell surface molecule.

11. The method of any one of claims 1-2 and 4-10 wherein the cell is a secretory cell, a chondrocyte, an epithelial cell, a nerve cell, a muscle cell, a blood cell, an endothelial cell, a pericyte, a fibroblast, a glial cell, or a dendritic cell.

12. The method of any one of claims 1-2 and 4-10, wherein the cell is a cancer cell, an immune cell, a bacterially-infected cell, a virally-infected cell, or a cell having an abnormal metabolic activity.

13. The method of any of claims 1-10 wherein the targeting ligand specifically binds to a cell surface molecule selected from the group consisting of transferrin receptor type 1, transferrin receptor type 2, the EGF receptor, HER2/Neu, a VEGF receptor, a PDGF receptor, an integrin, an NGF receptor, CD2, CD3, CD4, CD8, CD19, CD20, CD22, CD33, CD43, CD38, CD56, CD69, the asialoglycoprotein receptor (ASGPR), prostate-specific membrane antigen (PSMA), a folate receptor, and a sigma receptor.

14. The method of any one of claims 5-13, wherein T⁵ specifically binds to a cell surface molecule selected from the group consisting of transferrin receptor type 1, transferrin receptor type 2, the EGF receptor, HER2/Neu, a VEGF receptor, a PDGF receptor, an integrin, an NGF receptor, CD2, CD3, CD4, CD8, CD19, CD20, CD22, CD33, CD43, CD38, CD56, CD69, the asialoglycoprotein receptor (ASGPR), prostate-specific membrane antigen (PSMA), a folate receptor, and a sigma receptor.

15. The method of any of claims 1-14, wherein the targeting ligand comprises a small molecule targeting moiety.

16. The method of claim 15, wherein the small molecule targeting moiety is a sugar, a vitamin, a bisphosphonate, or an analogue thereof.

17. The method of claim 16, wherein the sugar is selected from lactose, galactose, N-acetyl galactosamine (NAG), N-acetyl galactosamine derivatives, and mannose-6-phosphate (M6P).

18. The method of claim 16, wherein the vitamin is folate.

19. The method of any of claims 1-14, wherein the targeting ligand comprises a protein.

20. The method of claim 19, wherein the protein is an antibody, a peptide aptamer, or a protein derived from a natural ligand of the cell surface molecule.

21. The method of any of claims 1-14, wherein the targeting ligand comprises a peptide.

22. The method of claim 21, wherein peptide is an integrin-binding peptide, a LOX-1-binding peptide, and epidermal growth factor (EGF) peptide, a neurotensin peptide, an NL4 peptide, or a YIGSR laminin peptide.

23. The method of any one of claims 1-2 and 4-22 wherein the cell is a hepatocyte.

24. The method of claim 23, wherein the targeting ligand specifically binds to the asialoglycoprotein receptor (ASGPR).

25. The method of claim 24, wherein the targeting ligand comprises an N-acetylgalactosamine (NAG) residue.

26. The method of any one of claims 1-25 wherein M² is selected from the group consisting of:
2,2,3,3,4,4,4-heptafluorobutyl methacrylate residue,
3,3,4,4,5,6,6,6-octafluoro-5(trifluoromethyl)hexyl methacrylate residue,
2,2,3,3,4,4,5,5,6,6,7,7,8,8,8-pentadecafluorooctyl 2-methylacrylate residue,
3,3,4,4,5,5,6,6,6-nonafluorohexyl methacrylate residue,
3,3,4,4,5,5,6,6,7,7,8,8,8-tridecafluorooctyl methacrylate residue,
1,1,1-trifluoro-2-(trifluoromethyl)-2-hydroxy-4-methyl-5-pentyl methacrylate residue, 2-[(1′,1′, 1′-trifluoro-2′-(trifluoro methyl)-2′-hydroxy)propyl]-3-norbornyl methacrylate residue, 2-ethylhexyl methacrylate residue,
butyl methacrylate residue,
hexyl methacrylate residue,
octyl methacrylate residue,
n-decyl methacrylate residue,
lauryl methacrylate residue,
myristyl methacrylate residue,
stearyl methacrylate residue,
cholesteryl methacrylate residue,
ethylene glycol phenyl ether methacrylate residue,
2-propenoic acid, 2-methyl-, 2-phenylethyl ester residue,
2-propenoic acid, 2-methyl-, 2-[[(1,1-dimethylethoxy)carbonyl]amino]ethyl ester residue,
2-propenoic acid, 2-methyl-, 2-(1H-imidazol-1-yl)ethyl ester residue,
2-propenoic acid, 2-methyl-, cyclohexyl ester residue,
2-propenoic acid, 2-methyl-, 2-[bis(1-methylethyl)amino]ethyl ester residue,
2-propenoic acid, 2-methyl-, 3-methylbutyl ester residue,
neopentyl methacrylate residue,
tert-butyl methacrylate residue,
3,3,5-trimethyl cyclohexyl methacrylate residue,
2-hydroxypropyl methacrylate residue,
5-nonyl methacrylate residue,
2-butyl-1-octyl methacrylate residue,
2-hexyl-1-decyl methacrylate residue, and
2-(tert-butyl amino)ethyl methacrylate residue.

27. The method of claim 26, wherein PEGMA has 4-5 ethylene glycol units or 7-8 ethylene glycol units.

28. The method of claim 27, wherein L comprises a polyethylene glycol (PEG) moiety having 2-20 ethylene glycol units.

29. The method of any one of claims 1-25, wherein the membrane destabilizing polymer is a polymer of formula (XXI): 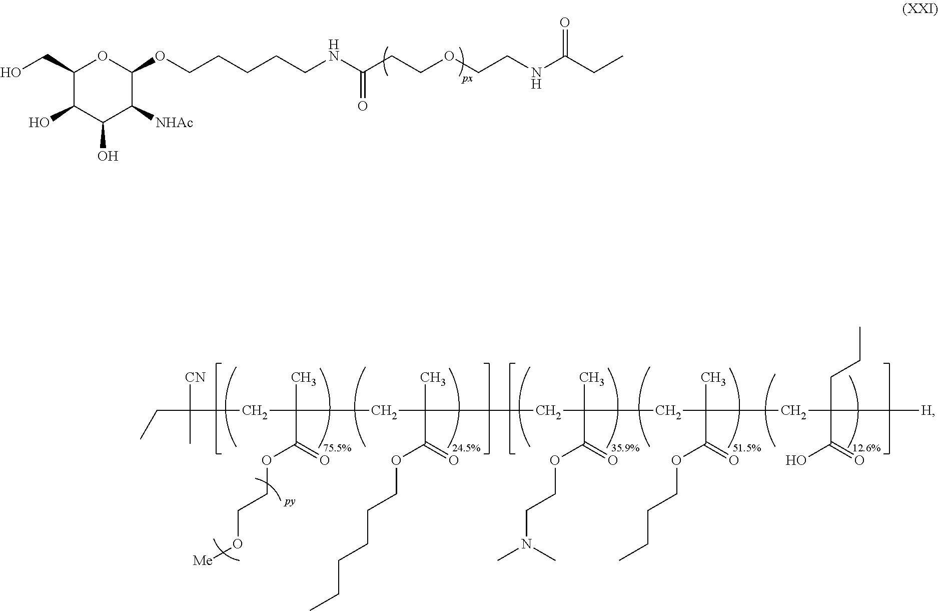
wherein px is an integer of from about 2 to about 50, e.g., from about 2 to about 20, e.g., from 4 to 12, e.g., from about 8 to about 16, e.g., px is about 12, and py is an integer of from about 2 to about 20, e.g., py is an integer of from about 2 to about 10, e.g., py is an integer of from about 4 to about 5 (e.g., 4 or 5).

30. The method of any one of claims 1-29, wherein the compound of formula (X) is a compound of formula (I): 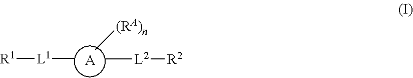
wherein:
R¹ a is targeting ligand;
L¹ is absent or a linking group;
L² is absent or a linking group;
R² is the nucleic acid;
the ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;
R^{B} is hydrogen, a protecting group, a covalent bond to a solid support, or a bond to a linking group that is bound to a solid support; and
n is 0, 1, 2, 3, 4, 5, 6, 7, 8, 9, or 10;
or a salt thereof.

31. The method of claim 30, wherein:
R¹ a is targeting ligand;
L¹ is absent or a linking group;
L² is absent or a linking group;
R² is the nucleic acid;
the ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, —C₁₋₂ alkyl-OR^{B} and C₁₋₈ alkyl that is optionally substituted with one or more groups independently selected from halo, hydroxy, and C₁₋₃ alkoxy;
R^{B} is hydrogen, a protecting group, a covalent bond to a solid support, or a bond to a linking group that is bound to a solid support; and
n is 0, 1, 2, 3, 4, 5, 6, 7, 8, 9, or 10.

32. The method of claim 30 wherein the compound of formula (I) is a compound of formula (Ia): 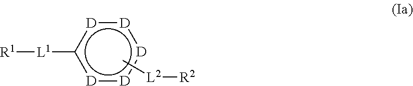
wherein:
each D is independently selected from the group consisting of 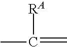
and —N═.

33. The method of claim 30, wherein the compound of formula (I) is a compound of formula (Ib): 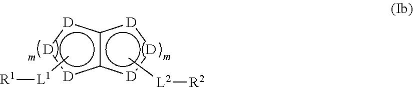
wherein:
each D is independently selected from the group consisting of 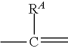
and —N═; and
each m is independently 1 or 2.

34. The method of claim 30, wherein the compound of formula (I) is a compound of formula (Ic): 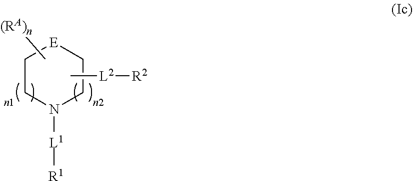
wherein:
E is —O— or —CH₂—;
n is selected from the group consisting of 0, 1, 2, 3, and 4; and
n1 and n2 are each independently selected from the group consisting of 0, 1, 2, and 3.

35. The method of claim 30 wherein the compound of formula (I) is a compound of formula (Ig): 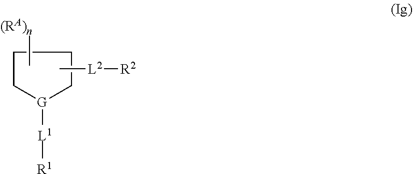
wherein:
G is —N— or —CH—;
L² is C₁₋₄ alkylene-O— that is optionally substituted with hydroxyl or halo; and
n is 0, 1, 2, 3, 4, 5, 6, or 7.

36. The method of any one of claims 1-29, wherein the compound of formula (X) is a compound of formula (XXX): 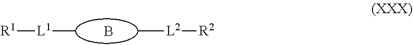
wherein:
R¹ a is targeting ligand;
L¹ is absent or a linking group;
L² is absent or a linking group;
R² is a nucleic acid;
B is divalent and is selected from the group consisting of: 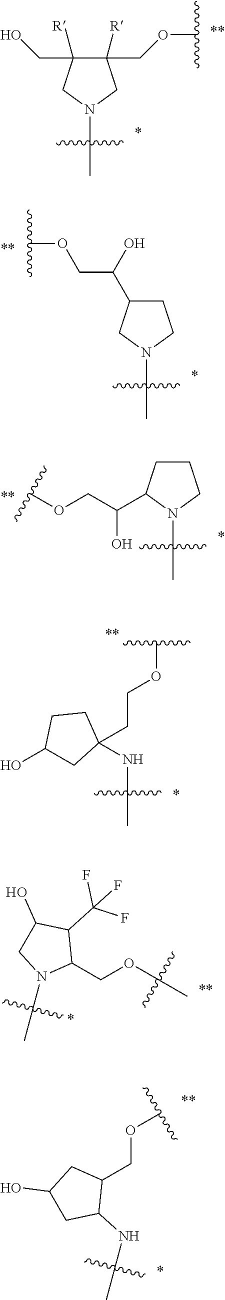 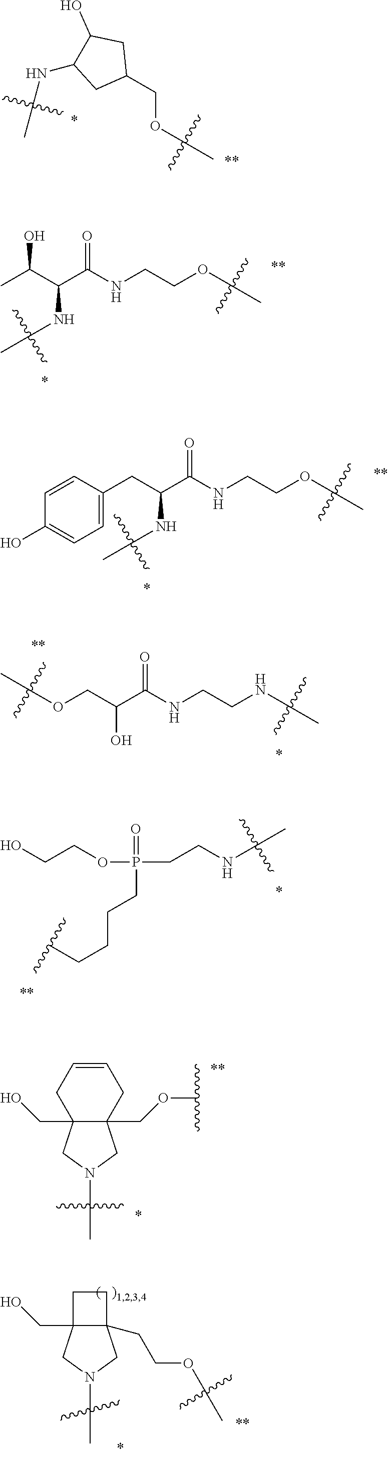 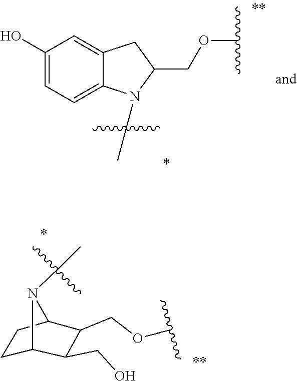
wherein:
each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;
the valence marked with * is attached to L¹ or is attached to R¹ if L¹ is absent; and
the valence marked with ** is attached to L² or is attached to R² if L² is absent.

37. The method of claim 32, the compound of formula (I) is selected from the group consisting of: 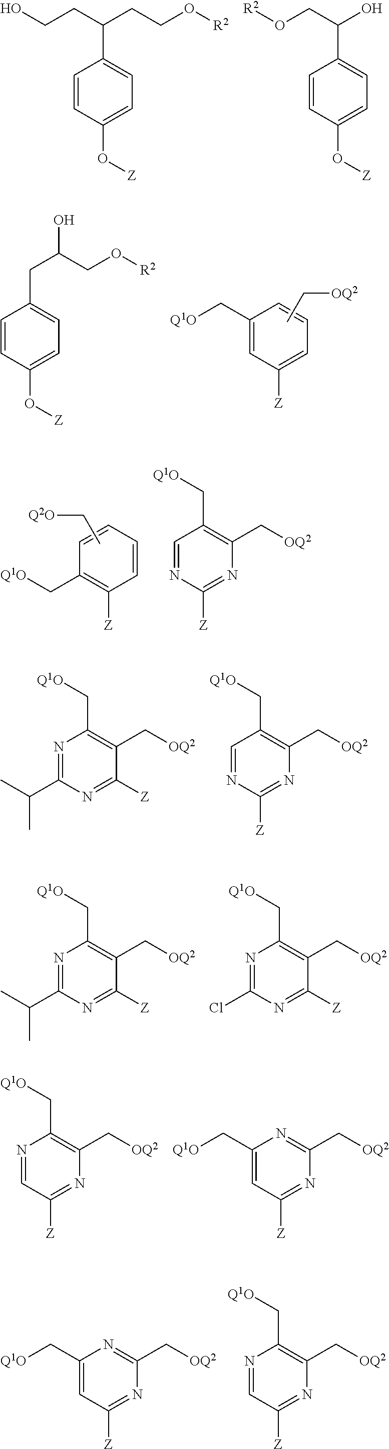 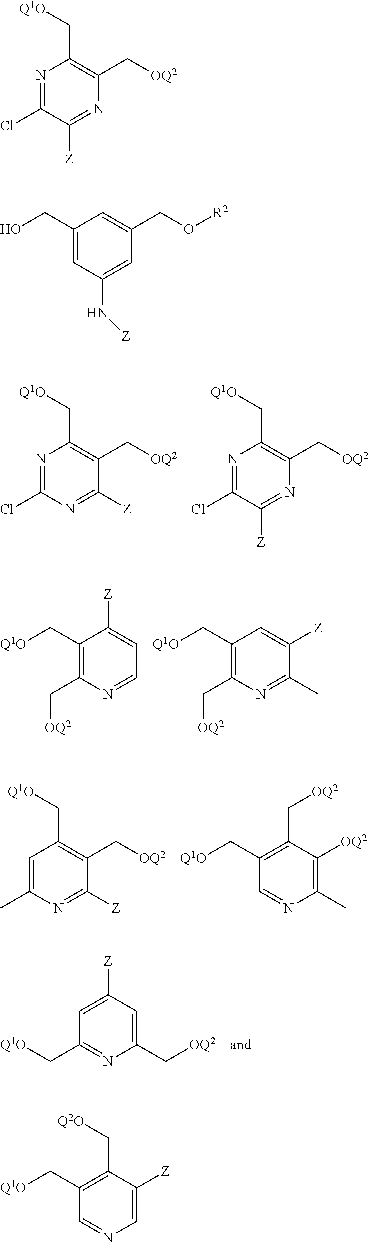
wherein:
Q¹ is hydrogen and Q² is R²; or Q¹ is R² and Q² is hydrogen; and
Z is -L¹-R¹.

38. The method of claim 33, wherein the compound of formula (I) is selected from the group consisting of: 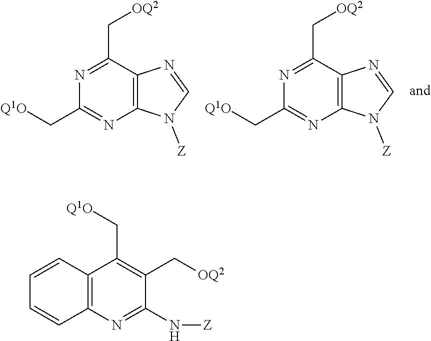
wherein:
Q¹ is hydrogen and Q² is R^{Z}; or Q¹ is W² and Q² is hydrogen; and
Z is -L¹-R¹.

39. The method of claim 34, wherein the compound of formula (I) is selected from the group consisting of: 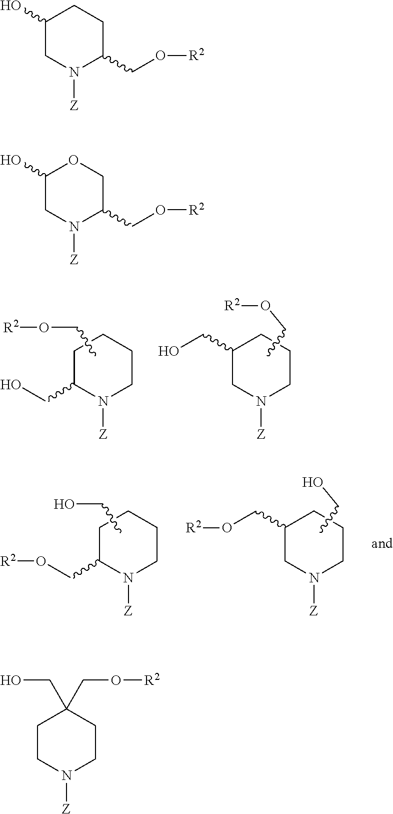
wherein: Z is -L¹-R¹.

40. The method of claim 30, wherein the compound of formula (I) is selected from the group consisting of: 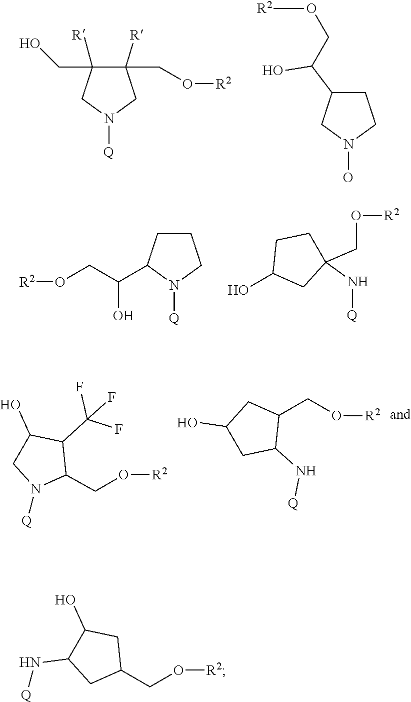
wherein Q is -L¹-R¹; and
R′ is C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

41. The method of claim 30, wherein the compound of formula (I) is selected from the group consisting of: 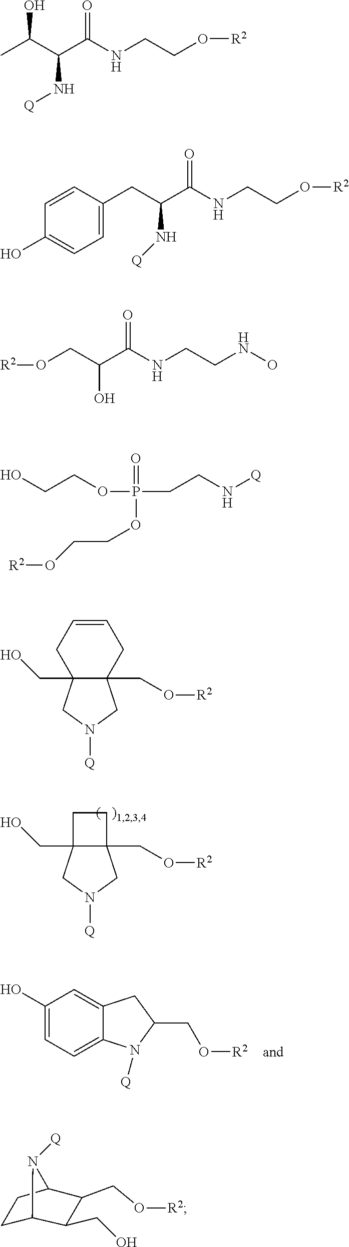
wherein Q is -L¹-R¹.

42. The method of any one of claims 30-31, wherein A is absent, phenyl, pyrrolidinyl, or cyclopentyl.

43. The method of any one of claims 34-35, wherein each R^{A} is independently hydroxy or C₁₋₈ alkyl that is optionally substituted with hydroxyl.

44. The method of any one of claims 34-35, wherein each R^{A} is independently selected from the group consisting of hydroxy, methyl and —CH₂OH.

45. The method of any one of claims 30-44, wherein L¹ is connected to R¹ through —NH—, —O—, —S—, —(C═O)—, —(C═O)—NH—, —NH—(C═O)—, —(C═O)—O—, —NH—(C═O)—NH—, or —NH—(SO₂)—.

46. The method of any one of claims 30-44, wherein L¹ is selected from the group consisting of:

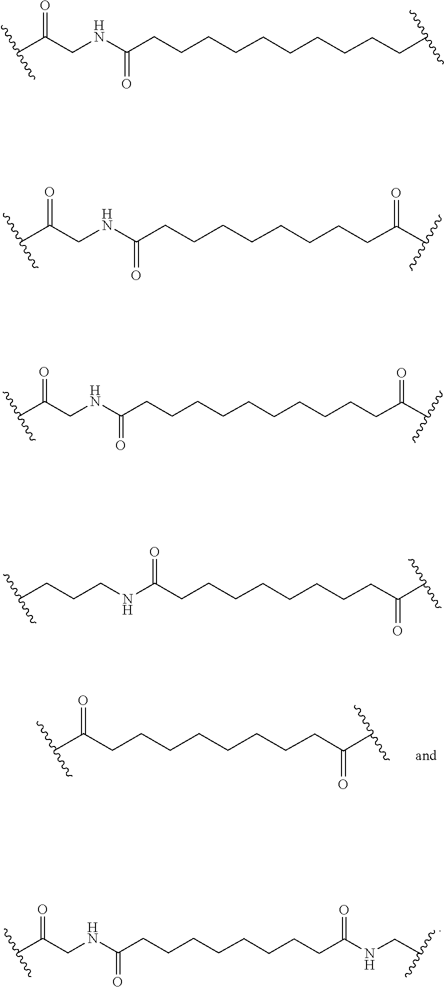 [SMILES: *CCCCCCCCCCC(=O)NCC(*)=O ; *C(=O)CCCCCCCCC(=O)NCC(*)=O ; *C(=O)CCCCCCCCCCC(=O)NCC(*)=O ; *CCCNC(=O)CCCCCCCCC(*)=O ; *C(=O)CCCCCCCCC(*)=O ; *CNC(=O)CCCCCCCCC(=O)NCC(*)=O]

47. The method of any one of claims 30-44, wherein L¹ is selected from the group consisting of:

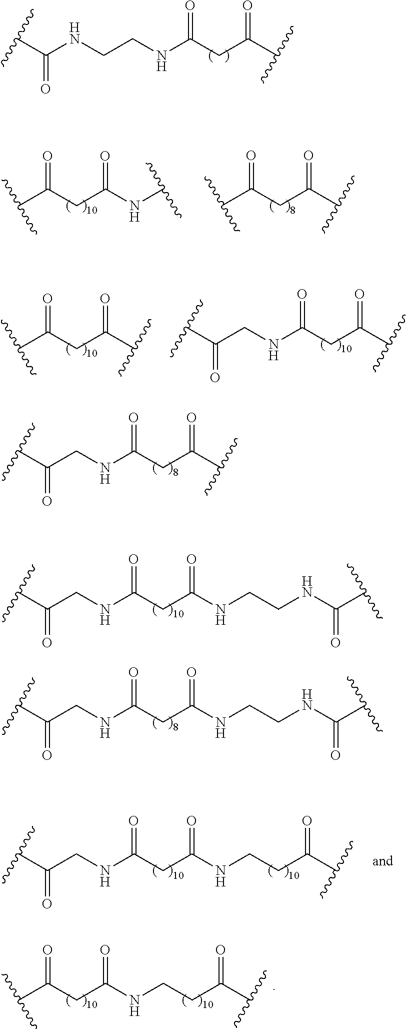

48. The method of any one of claims 30-44, wherein L¹ is selected from the group consisting of:

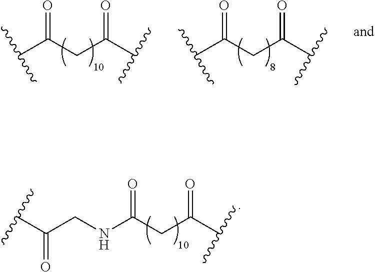

49. The method of any one of claims 30-44, wherein L¹ is connected to B through a linkage selected from the group consisting of: —O—, —S—, —(C═O)—, —(C═O)—NH—, —NH—(C═O), —(C═O)—O—, —NH—(C═O)—NH—, or —NH—(SO₂)—.

50. The method of any one of claims 30-44, wherein L¹ is selected from the group consisting of:

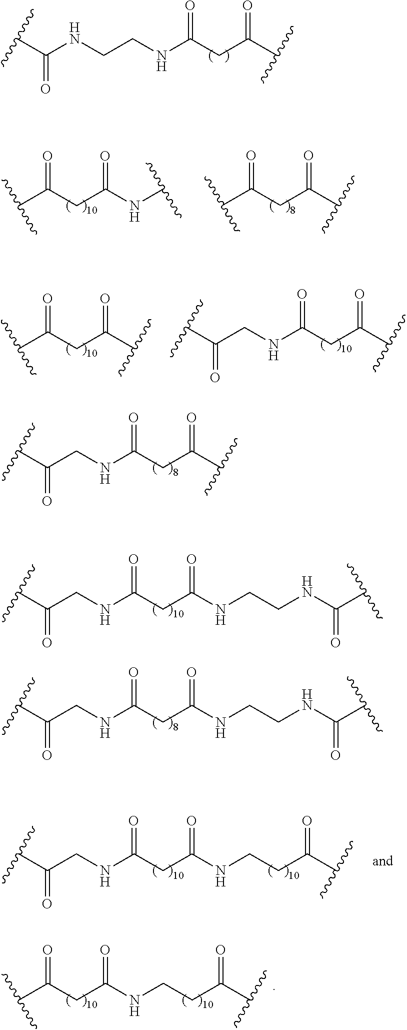

51. The method of any one of claims 30-50, wherein L² is connected to R² through —O—.

52. The method of any one of claims 30-50, wherein L² is C₁₋₄ alkylene-O— that is optionally substituted with hydroxy.

53. The method of any one of claims 30-50, wherein L² is —CH₂O—, —CH₂CH₂O—, or —CH(OH)CH₂O—.

54. The method of any one of claims 30-50, wherein L² is —CH₂—O— or —CH₂—CH₂—O—.

55. The method of any one of claims 30-50, wherein L² is absent.

56. The method of any one of claims 30-44, wherein L¹ and L² are independently a divalent, branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

57. The method of any one of claims 30-44, wherein L¹ and L² are independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

58. The method of any one of claims 30-44, wherein L¹ and L² are independently, a divalent, branched or unbranched, saturated or unsaturated, hydrocarbon chain, having from 1 to 14 carbon atoms, wherein one or more (e.g. 1, 2, 3, or 4) of the carbon atoms in the hydrocarbon chain is optionally replaced —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

59. The method of any one of claims 30-58, wherein the targeting ligand R¹ comprises 3-8 saccharides.

60. The method of any one of claims 30-58, wherein the targeting ligand R¹ comprises 3-6 saccharides.

61. The method of any one of claims 30-58, wherein the targeting ligand R¹ comprises 3-4 saccharides.

62. The method of any one of claims 30-58, wherein the targeting ligand R¹ comprises 3 saccharides.

63. The method of any one of claims 30-58, wherein the targeting ligand R¹ comprises 4 saccharides.

64. The method of any one of claims 30-58, wherein the targeting ligand R¹ has the following formula: 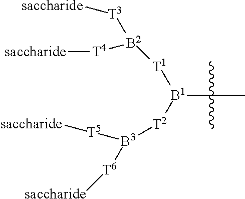
wherein:
B¹ is a trivalent group comprising about 1 to about 20 atoms and is covalently bonded to L¹, T¹, and T².
B² is a trivalent group comprising about 1 to about 20 atoms and is covalently bonded to T¹, T³, and T⁴;
B³ is a trivalent group comprising about 1 to about 20 atoms and is covalently bonded to T², T⁵, and T⁶;
T¹ is absent or a linking group;
T² is absent or a linking group;
T³ is absent or a linking group;
T⁴ is absent or a linking group;
T⁵ is absent or a linking group; and
T⁶ is absent or a linking group.

65. The method of claim 64, wherein one of T¹ and T² is absent.

66. The method of claim 64, wherein both T¹ and T² are absent.

67. The method of claim 64, herein each of T¹, T², T³, T⁴, T, and r is independently absent or a branched or unbranched, saturated or unsaturated, hydrocarbon chain, having from 1 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

68. The method of claim 64, wherein each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C1-C6)alkyl, and wherein the hydrocarbon chain, is optionally substituted with one or more (e.g. 1, 2, 3, or 4) substituents selected from (C1-C6)alkoxy, (C3-C6)cycloalkyl, (C1-C6)alkanoyl, (C1-C6)alkanoyloxy, (C1-C6)alkoxycarbonyl, (C1-C6)alkylthio, azido, cyano, nitro, halo, hydroxy, oxo (═O), carboxy, aryl, aryloxy, heteroaryl, and heteroaryloxy.

69. The method of claim 64, wherein each of T¹, T², T³, T⁴, T⁵, and T⁶ is independently absent or a branched or unbranched, saturated or unsaturated, hydrocarbon chain, having from 1 to 50 carbon atoms, wherein one or more of the carbon atoms in the hydrocarbon chain is optionally replaced by —O— or —NR^{X}—, and wherein R^{X} is hydrogen or (C₁-C₆)alkyl, and wherein the hydrocarbon chain, is optionally substituted with one or more (e.g. 1, 2, 3, or 4) substituents selected from halo, hydroxy, and oxo (═O).

70. The method of claim 64, wherein each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

71. The method of claim 64, wherein each of T¹, T², T³, T⁴, T⁵, and T⁶ is independently absent or a branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O— and wherein the hydrocarbon chain, is optionally substituted with one or more (e.g. 1, 2, 3, or 4) substituents selected from halo, hydroxy, and oxo (═O).

72. The method of any one of claims 64-66, wherein at least one of T³, T⁴, T⁵, and T⁶ is: 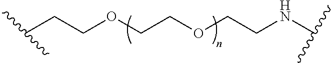
wherein:
n=1, 2, 3.

73. The method of any one of claims 64-66, wherein each of T³, T⁴, T⁵, and T⁶ is independently selected from the group consisting of: 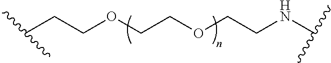
wherein:
n=1, 2, 3.

74. The method of claim 64, wherein at least one of T¹ and T² is glycine.

75. The method of claim 64, wherein each of T¹ and T² is glycine.

76. The method of any one of claims 64-75, wherein B¹ is a trivalent group comprising 1 to 15 atoms and is covalently bonded to L¹, T¹, and T².

77. The method of any one of claims 64-75, wherein B¹ is a trivalent group comprising 1 to 10 atoms and is covalently bonded to L¹, T¹, and T².

78. The method of any one of claims 64-75, wherein B¹ comprises a (C₁-C₆)alkyl.

79. The method of any one of claims 64-75, wherein B¹ comprises a C₃₋₈ cycloalkyl.

80. The method of any one of claims 64-75, wherein B¹ comprises a silyl group.

81. The method of any one of claims 64-75, wherein B¹ comprises a D- or L-amino acid.

82. The method of any one of claims 64-75, wherein B¹ comprises a saccharide.

83. The method of any one of claims 64-75, wherein B¹ comprises a phosphate group.

84. The method of any one of claims 64-75, wherein B¹ comprises a phosphonate group.

85. The method of any one of claims 64-75, wherein B¹ comprises an aryl.

86. The method of any one of claims 64-75, wherein B¹ comprises a phenyl ring.

87. The method of any one of claims 64-75, wherein B¹ is a phenyl ring.

88. The method of any one of claims 64-75, wherein B¹ is CH.

89. The method of any one of claims 64-75, wherein B¹ comprises a heteroaryl.

90. The method of any one of claims 64-75, wherein B¹ is:

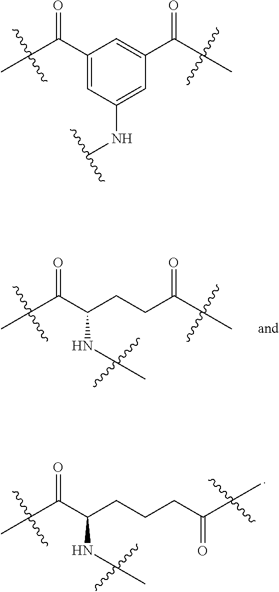 [SMILES: *Nc1cc(C(*)=O)cc(C(*)=O)c1 ; *N[C@@H](CCC(*)=O)C(*)=O ; *N[C@H](CCCC(*)=O)C(*)=O]

91. The method of any one of claims 64-90, wherein B² is a trivalent group comprising 1 to 15 atoms and is covalently bonded to T¹, T³, and T⁴.

92. The method of any one of claims 64-90, wherein B² is a trivalent group comprising 1 to 10 atoms and is covalently bonded to T¹, T³, and T⁴.

93. The method of any one of claims 64-90, wherein B² comprises a (C₁-C₆)alkyl.

94. The method of any one of claims 64-90, wherein B² comprises a C₃₋₈ cycloalkyl.

95. The method of any one of claims 64-90, wherein B² comprises a silyl group.

96. The method of any one of claims 64-90, wherein B² comprises a D- or L-amino acid.

97. The method of any one of claims 64-90, wherein B² comprises a saccharide.

98. The method of any one of claims 64-90, wherein B² comprises a phosphate group.

99. The method of any one of claims 64-90, wherein B² comprises a phosphonate group.

100. The method of any one of claims 64-90, wherein B² comprises an aryl.

101. The method of any one of claims 64-90, wherein B² comprises a phenyl ring.

102. The method of any one of claims 64-90, wherein B² is a phenyl ring.

103. The method of any one of claims 64-90, wherein B² is CH.

104. The method of any one of claims 64-90, wherein B² comprises a heteroaryl.

105. The method of any one of claims 64-90, wherein B² is selected from the group consisting of:

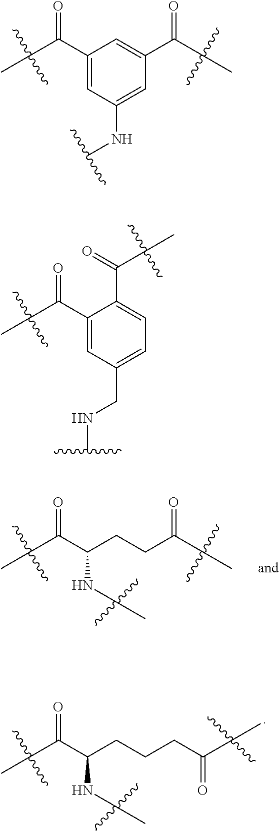 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

106. The method of any one of claims 64-105, wherein B³ is a trivalent group comprising 1 to 15 atoms and is covalently bonded to T², T⁵, and T⁶.

107. The method of any one of claims 64-105, wherein B³ is a trivalent group comprising 1 to 10 atoms and is covalently bonded to T², T⁵, and T⁶.

108. The method of any one of claims 64-105, wherein B³ comprises a (C₁-C₆)alkyl.

109. The method of any one of claims 64-105, wherein B³ comprises a C₃₋₈ cycloalkyl.

110. The method of any one of claims 64-105, wherein B³ comprises a silyl group.

111. The method of any one of claims 64-105, wherein B³ comprises a D- or L-amino acid.

112. The method of any one of claims 64-105, wherein B³ comprises a saccharide.

113. The method of any one of claims 64-105, wherein B³ comprises a phosphate group.

114. The method of any one of claims 64-105, wherein B³ comprises a phosphonate group.

115. The method of any one of claims 64-105, wherein B³ comprises an aryl.

116. The method of any one of claims 64-105, wherein B³ comprises a phenyl ring.

117. The method of any one of claims 64-105, wherein B³ is a phenyl ring.

118. The method of any one of claims 64-105, wherein B³ is CH.

119. The method of any one of claims 64-105, wherein B³ comprises a heteroaryl.

120. The method of any one of claims 64-105, wherein B³ is selected from the group consisting of:

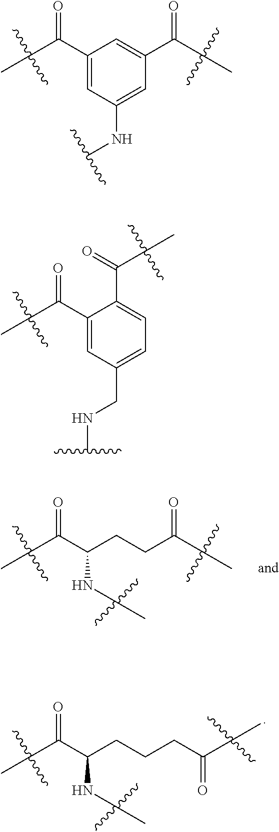 [SMILES: *Nc1cc(C(*)=O)cc(C(*)=O)c1 ; *NCc1ccc(C(*)=O)c(C(*)=O)c1 ; *N[C@@H](CCC(*)=O)C(*)=O ; *N[C@H](CCCC(*)=O)C(*)=O]

121. The method of any one of claims 30-58, wherein R¹ is:

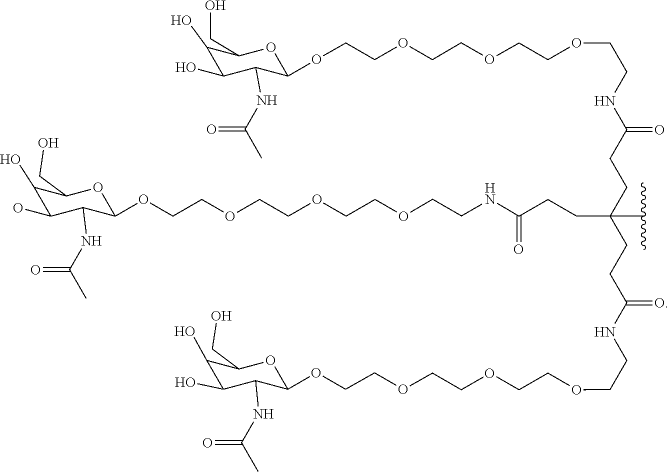

122. The method of any one of claims 30-58, wherein R¹ is: 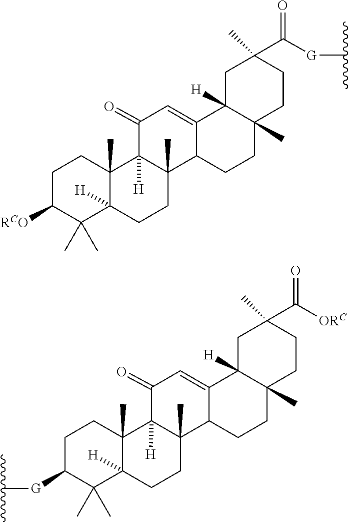
wherein:
G is —NH— or —O—;
R^{C} is hydrogen, (C₁-C₈)alkyl, (C₁-C₈)haloalkyl, (C₁-C₈)alkoxy, (C₁-C₆)alkanoyl, (C₃-C₂₀)cycloalkyl, (C₃-C₂₀)heterocycle, aryl, heteroaryl, monosaccharide, disaccharide or trisaccharide; and wherein the cycloalkyl, heterocyle, ary, heteroaryl and saccharide are optionally substituted with one or more groups independently selected from the group consisting of halo, carboxyl, hydroxyl, amino, (C₁-C₄)alkyl, (C₁-C₄)haloalkyl, (C₁-C₄)alkoxy and (C₁-C₄)haloalkoxy.

123. The method of claim 122, wherein R^{C} is:

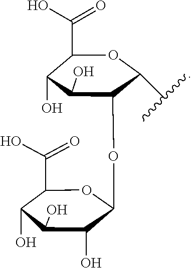

124. The method of claim 122, wherein R^{C} is:

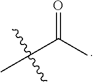 [SMILES: *C(C)=O]

125. The method of any one of claims 30-58, wherein R¹ is:

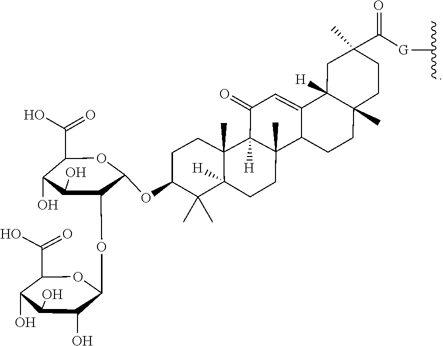

126. The method of any one of claims 122-125, wherein G is —NH—.

127. The method of any one of claims 30-58, wherein R¹ is:

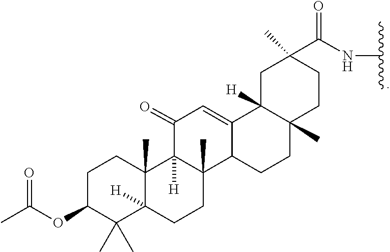

128. The method of any one of claims 30-58, wherein R is: 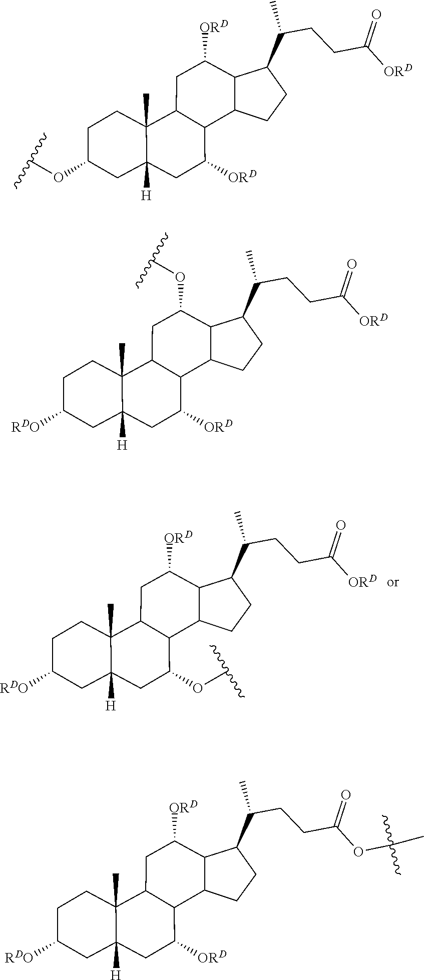
wherein each R^{D} is independently selected from the group consisting of hydrogen, (C₁-C₆)alkyl, (C₉-C₂₀)alkylsilyl, (R^{W})₃Si—, (C₂-C₆)alkenyl, tetrahydropyranyl, (C₁-C₆)alkanoyl, benzoyl, aryl(C₁-C₃)alkyl, TMTr (Trimethoxytrityl), DMTr (Dimethoxytrityl), MMTr (Monomethoxytrityl), and Tr (Trityl); and
each R^{W} is independently selected from the group consisting of (C₁-C₄)alkyl and aryl.

129. The method of any one of claims 30-58, wherein R¹ is selected from the group consisting of: 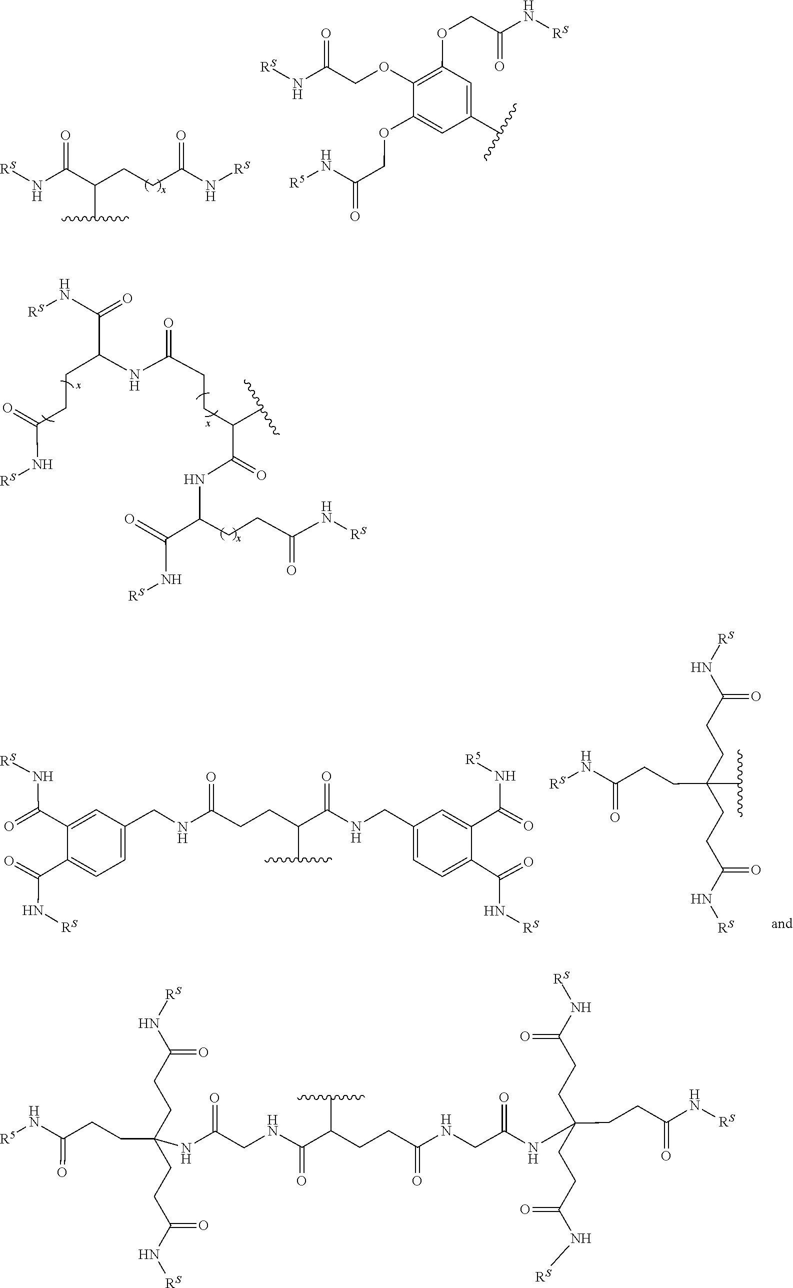
wherein:
R^{S} is 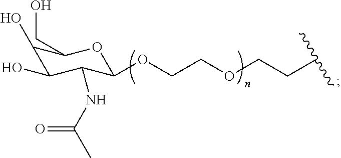
n is 2, 3, or 4; and
x is 1 or 2.

130. The method of any one of claims 30-58, wherein R¹ is —C(H)₍₃₋ₚ₎(L³-saccharide^{a})ₚ,
wherein each L³ is independently a linking group; p is 1, 2, or 3; and saccharide^{a} is a monosaccharide or disaccharide.

131. The method of claim 130, wherein each L³ is independently a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—N— or —S—, and wherein R^{X} is hydrogen or (C₁-C₆)alkyl, and wherein the hydrocarbon chain, is optionally substituted with one or more substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

132. The method of claim 130, wherein each L³ is independently a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

133. The method of claim 130, wherein L³ is:

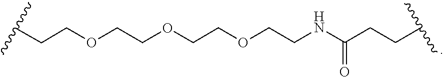 [SMILES: *CCOCCOCCOCCNC(=O)CC*]

134. The method of any one of claims 59-120, wherein each saccharide is independently: 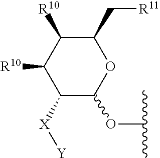
wherein:
X is NR³, and Y is selected from —(C═O)R⁴, —SO₂R⁵, and —(C═O)NR⁶R⁷; or X is —(C═O)— and Y is NR⁸R⁹;
R³ is hydrogen or (C₁-C₄)alkyl;
R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;
R¹⁰ is —OH, —NR⁸R⁹ or —F; and
R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

135. The method of any one of claims 59-120, wherein each saccharide is independently selected from the group consisting of:

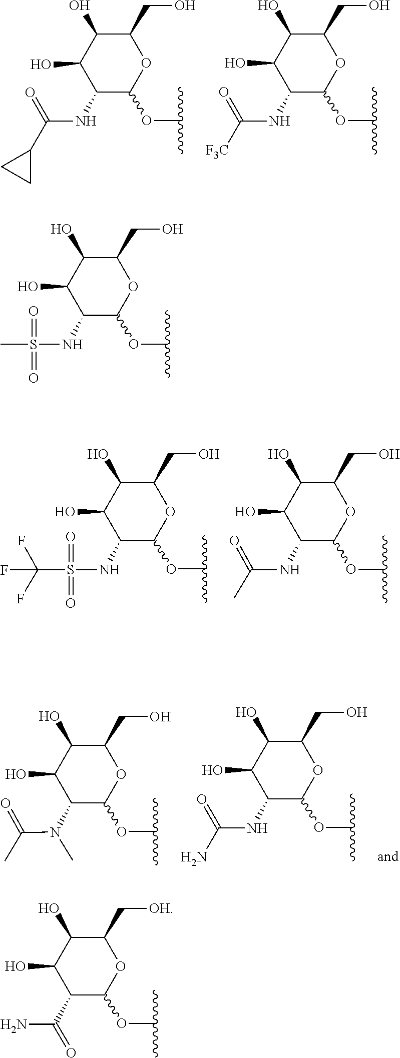

136. The method of any one of claims 59-120, wherein each saccharide is independently:

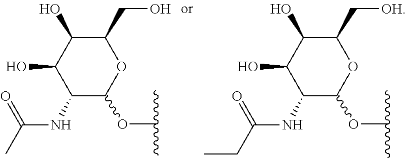 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

137. The method of any one of claims 1-136, wherein T⁵ is: 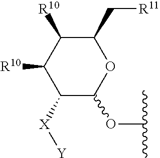
wherein:
X is NR³, and Y is selected from —(C═O)R⁴, —SO₂R⁵, and —(C═O)NR⁶R⁷; or X is —(C═O)— and Y is NR⁸R⁹;
R³ is hydrogen or (C₁-C₄)alkyl;
R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;
R¹⁰ is —OH, —NR⁸R⁹ or —F; and
R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

138. The method of any one of claims 1-136, wherein T⁵ is selected from the group consisting of:

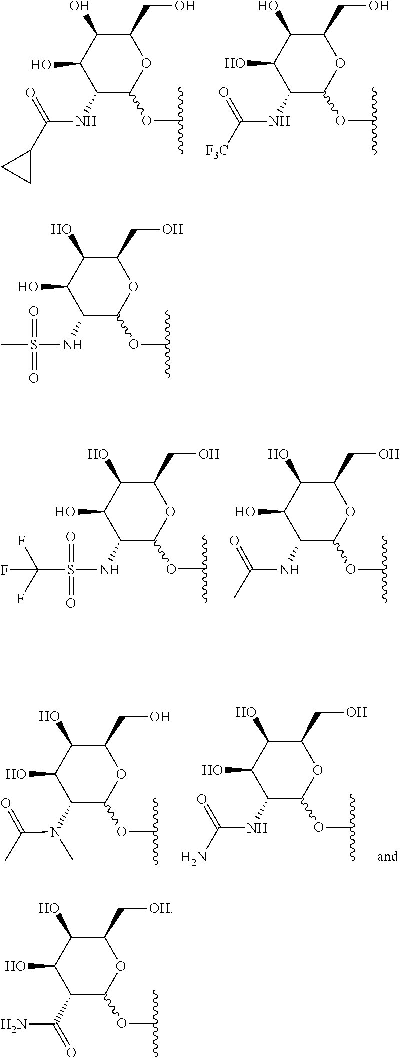

139. The method of any one of claims 1-136, wherein T⁵ is:

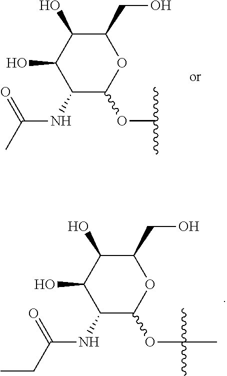 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

140. The method of any one of claims 130-133, wherein saccharide^{a} is: 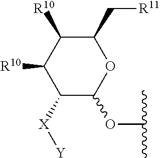
wherein:
X is NR³, and Y is selected from —(C═O)R⁴, —SO₂R⁵, and —(C═O)NR⁶R⁷; or X is —(C═O)— and Y is NR⁸R⁹;
R³ is hydrogen or (C₁-C₄)alkyl;
R⁴, R⁵, R⁶, R⁷, R⁸ and R⁹ are each independently selected from the group consisting of hydrogen, (C₁-C₈)alkyl, (C₁-C₈)haloalkyl, (C₁-C₈)alkoxy and (C₃-C₆)cycloalkyl that is optionally substituted with one or more groups independently selected from the group consisting of halo, (C₁-C₄)alkyl, (C₁-C₄)haloalkyl, (C₁-C₄)alkoxy and (C₁-C₄)haloalkoxy;
R¹⁰ is —OH, —NR⁸R⁹ or —F; and
R¹¹ is —OH, —NR⁸R⁹, —F or 5 membered heterocycle that is optionally substituted with one or more groups independently selected from the group consisting of halo, hydroxyl, carboxyl, amino, (C₁-C₄)alkyl, (C₁-C₄)haloalkyl, (C₁-C₄)alkoxy and (C₁-C₄)haloalkoxy.

141. The method of any one of claims 130-133, wherein saccharide^{a} is selected from the group consisting of:

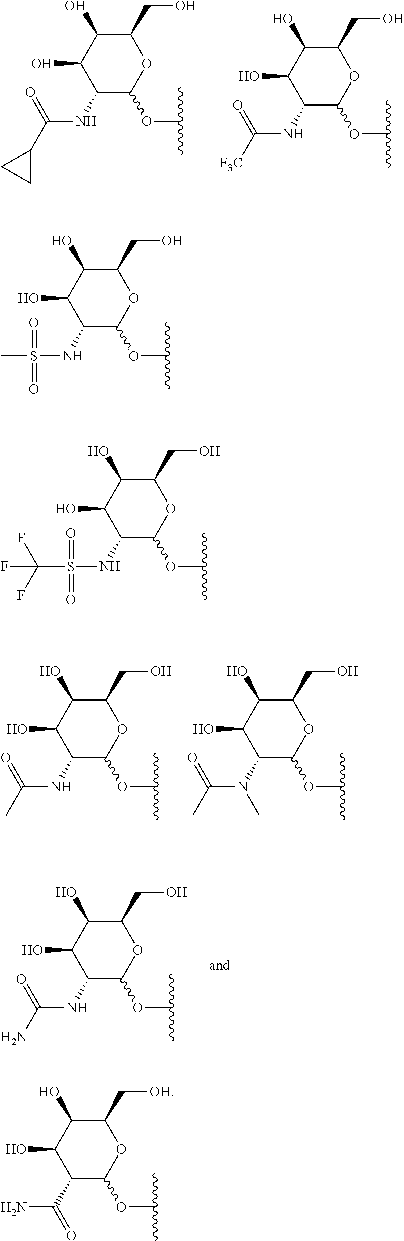

142. The method of any one of claims 130-133, wherein saccharide^{a} is:

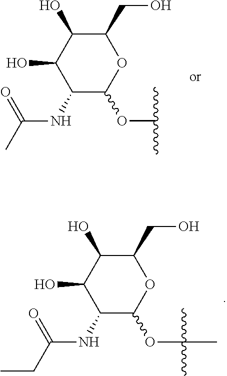 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

143. The method of claim 30, wherein the compound of formula (I) is selected from the group consisting of:

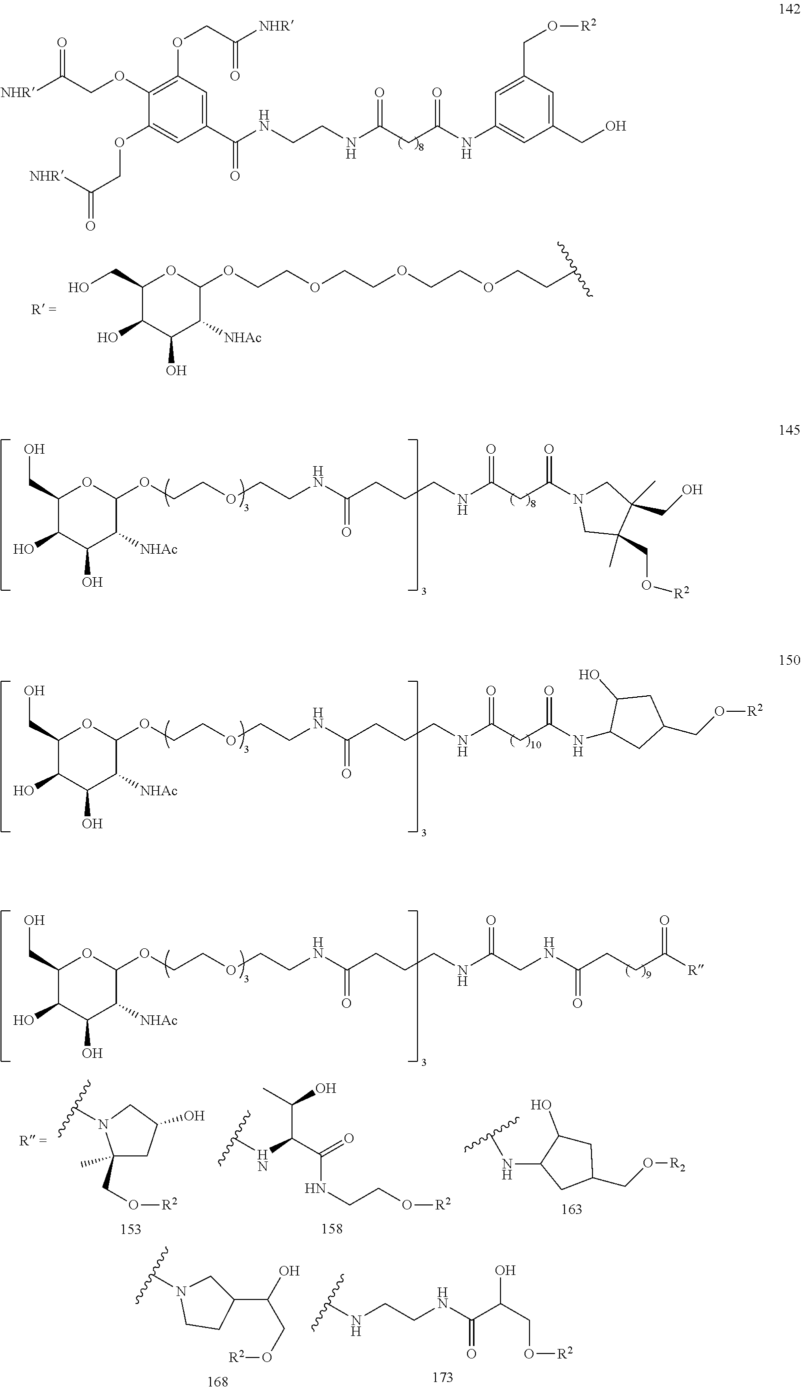

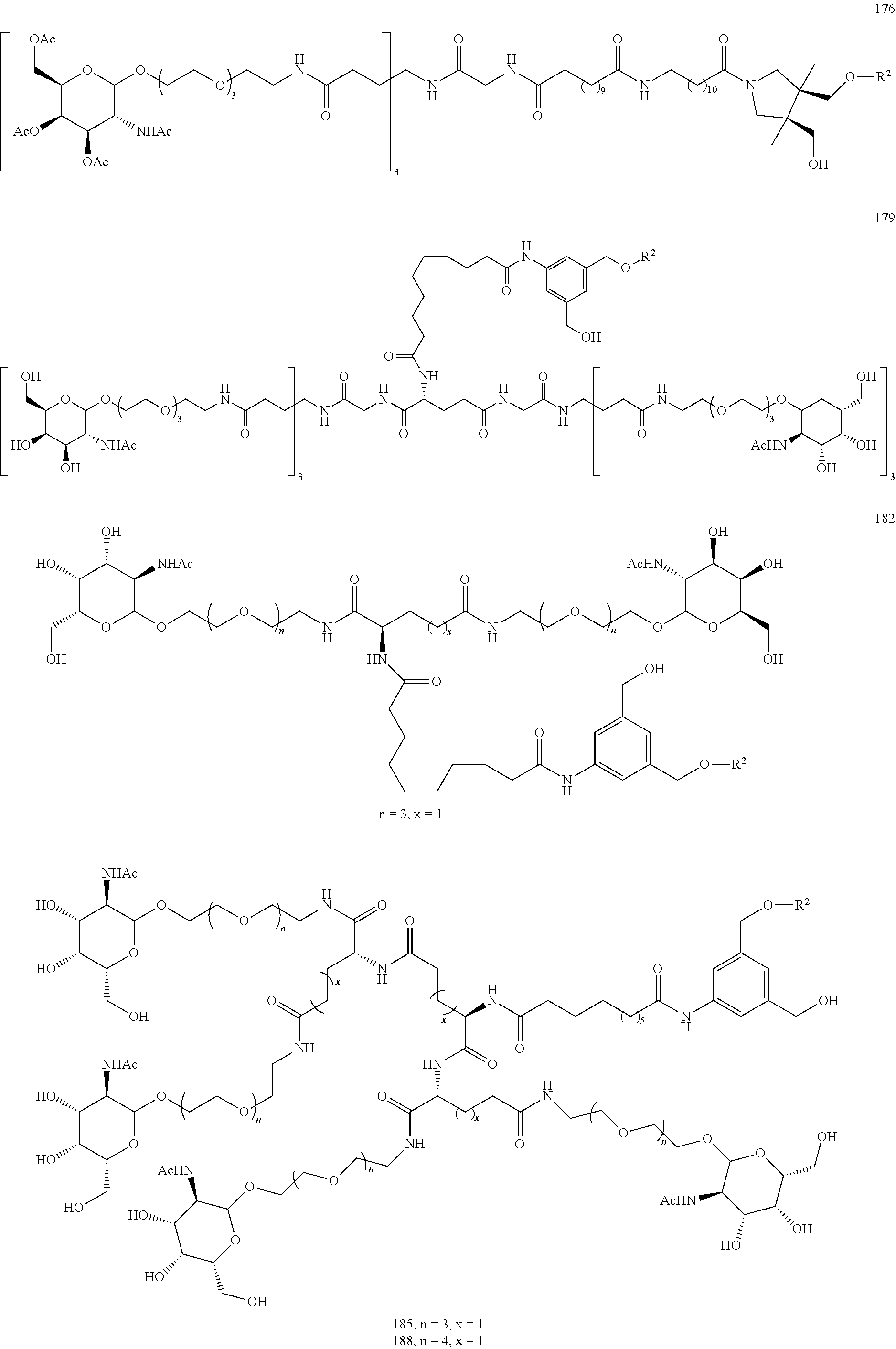

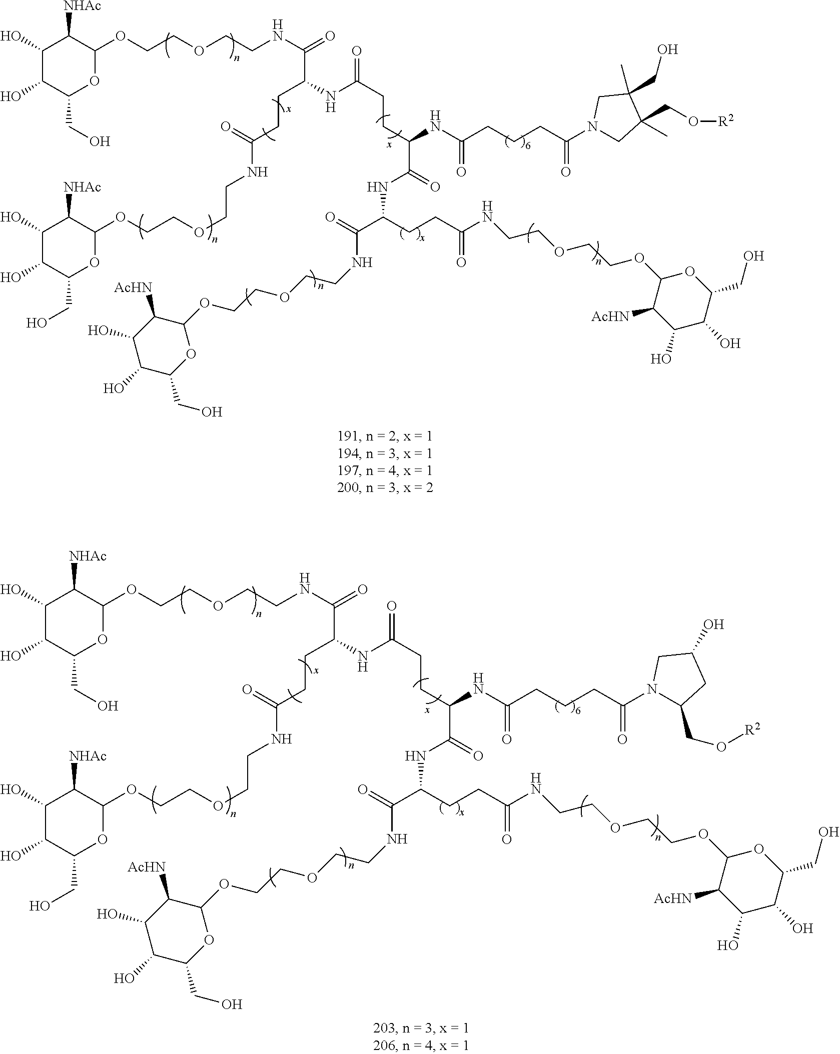

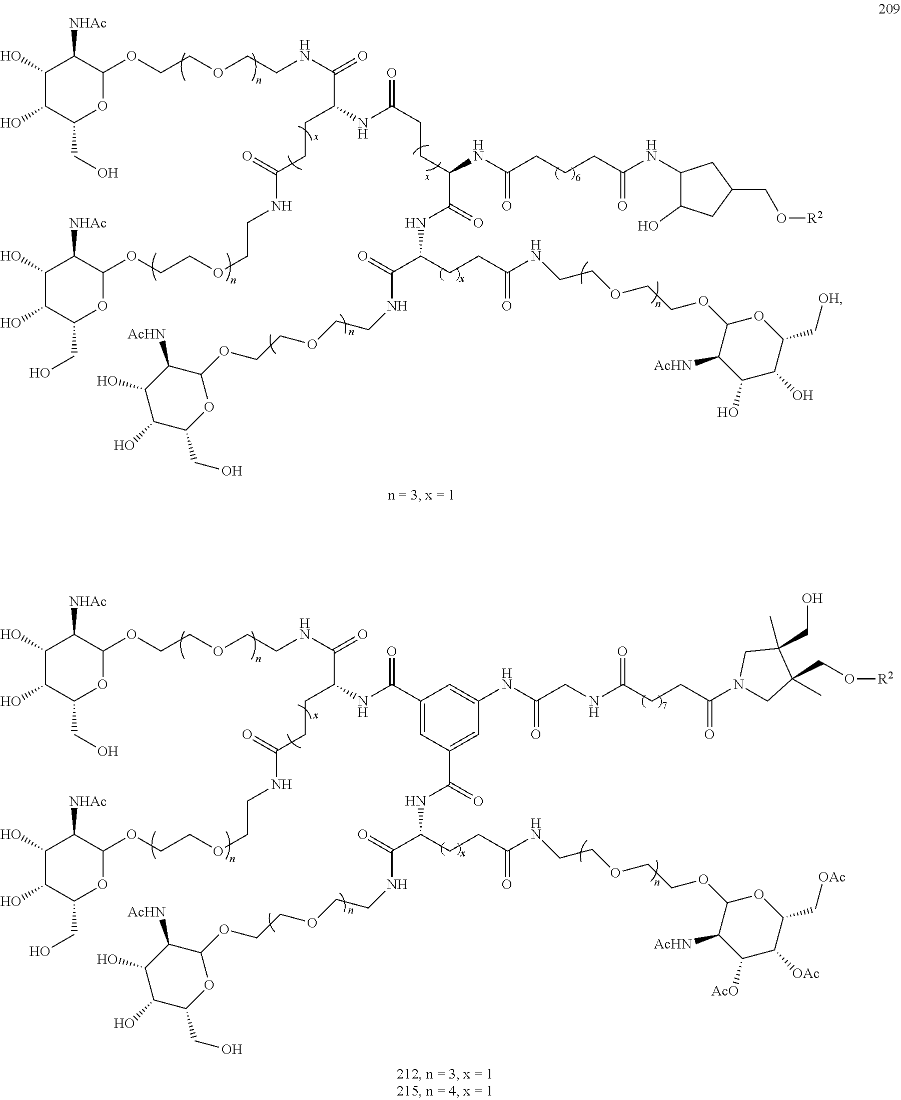

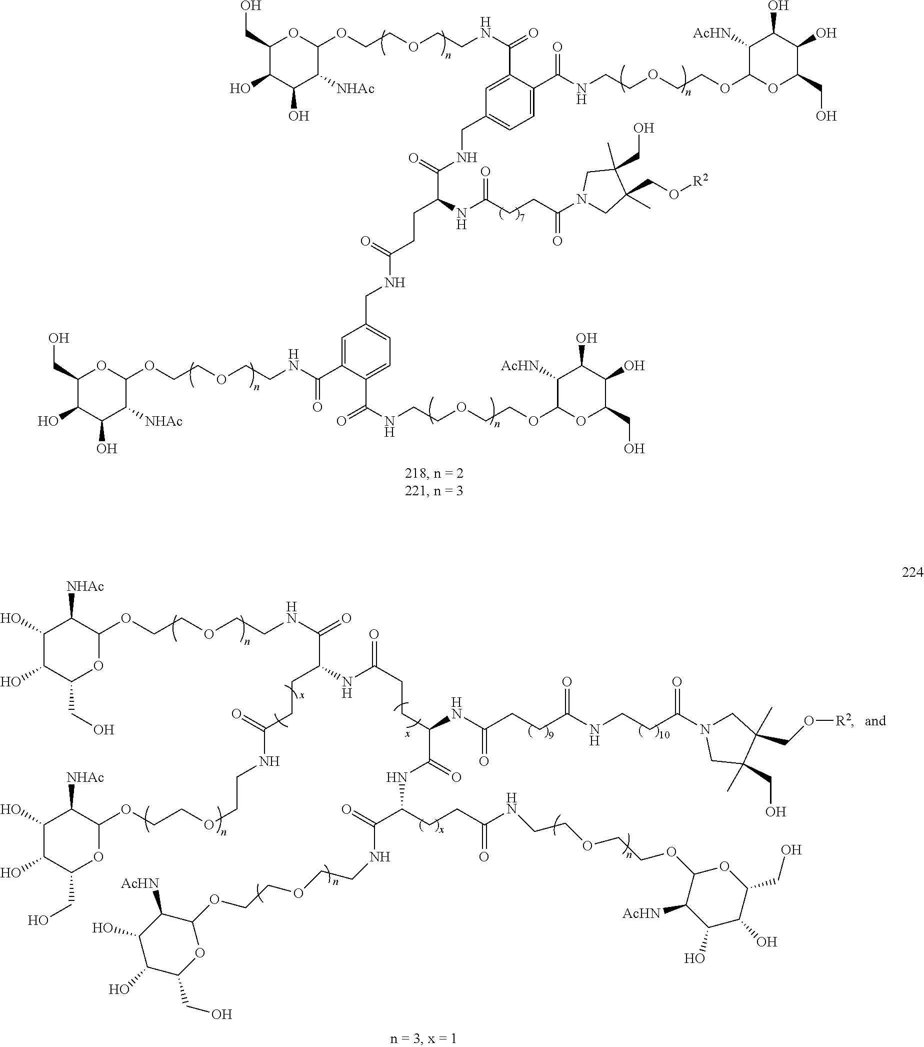

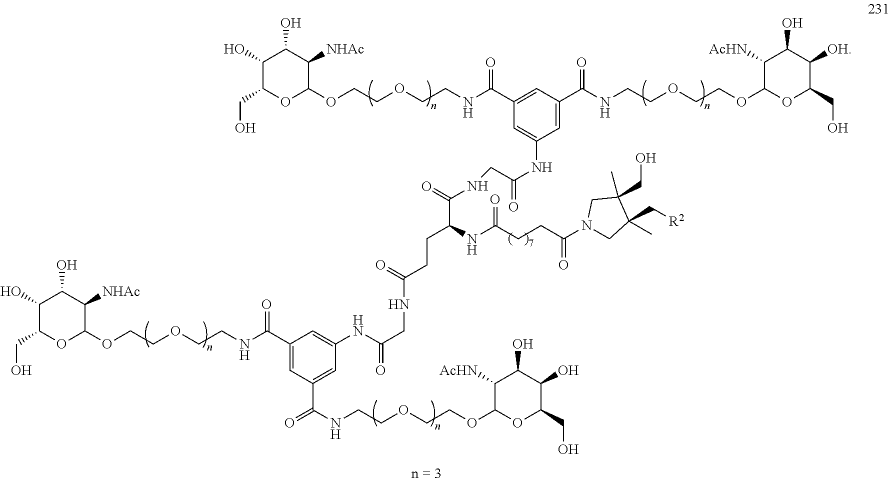

144. The method of claim 30, wherein the compound of formula (I) is a compound formula (Id): 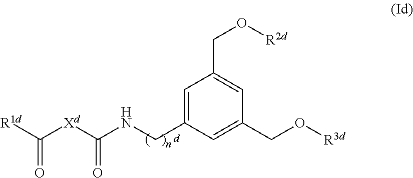
wherein:
R^{1d} is selected from: 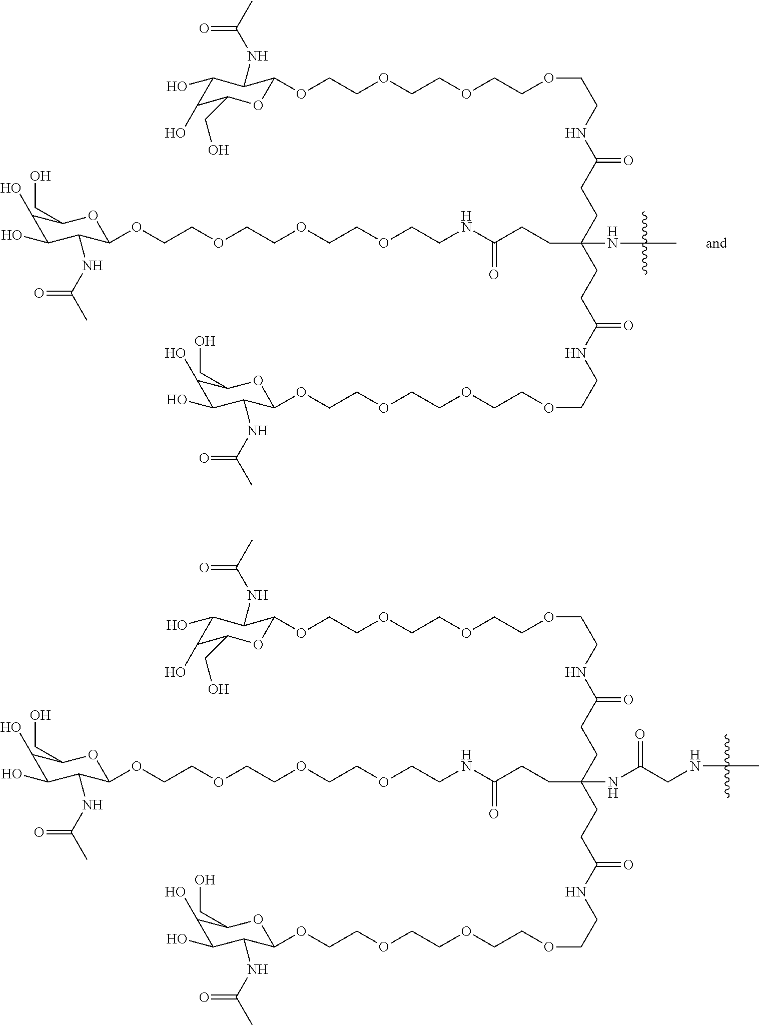 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O ; *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]
X^{d} is C₂₋₁₀ alkylene;
n^{d} is 0 or 1;
R^{2d} is a nucleic acid; and
R^{3d} is H.

145. The method of claim 144, wherein R^{1d} is:

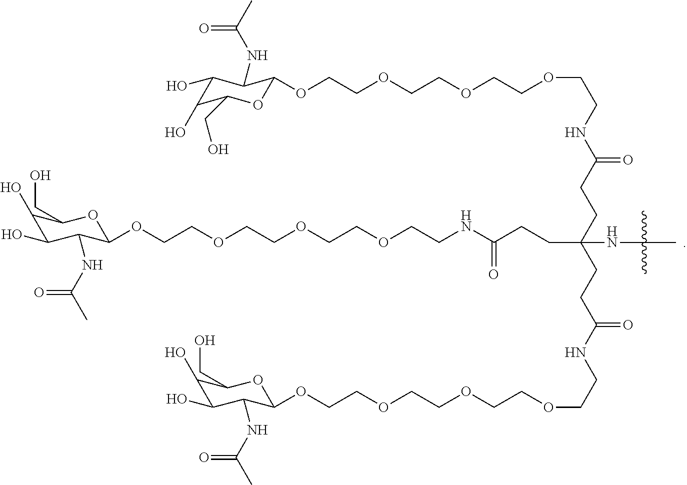 [SMILES: *NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

146. The method of claim 144, wherein R^{1d} is:

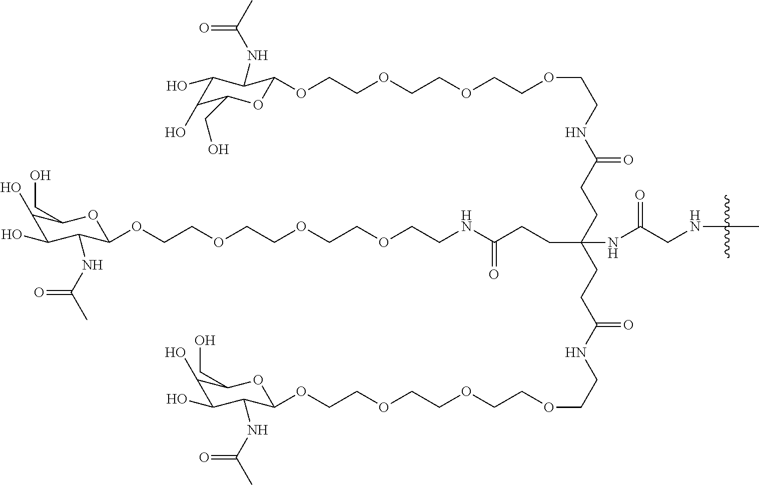 [SMILES: *NCC(=O)NC(CCC(=O)NCCOCCOCCOCCOC1OC(CO)C(O)C(O)C1NC(C)=O)(CCC(=O)NCCOCCOCCOCCOC1OC(CO)C(O)C(O)C1NC(C)=O)CCC(=O)NCCOCCOCCOCCOC1OC(CO)C(O)C(O)C1NC(C)=O]

147. The method of any one of claims 144-146, wherein X^{d} is C₈alkylene.

148. The method of any one of claims 144-146, wherein n^{d} is 0.

149. The method of any one of claims 144-148, wherein R^{3d} is H.

150. The method of any one of claims 1-29, wherein the compound of formula (X) is a compound of formula (I) that is selected from the group consisting of:

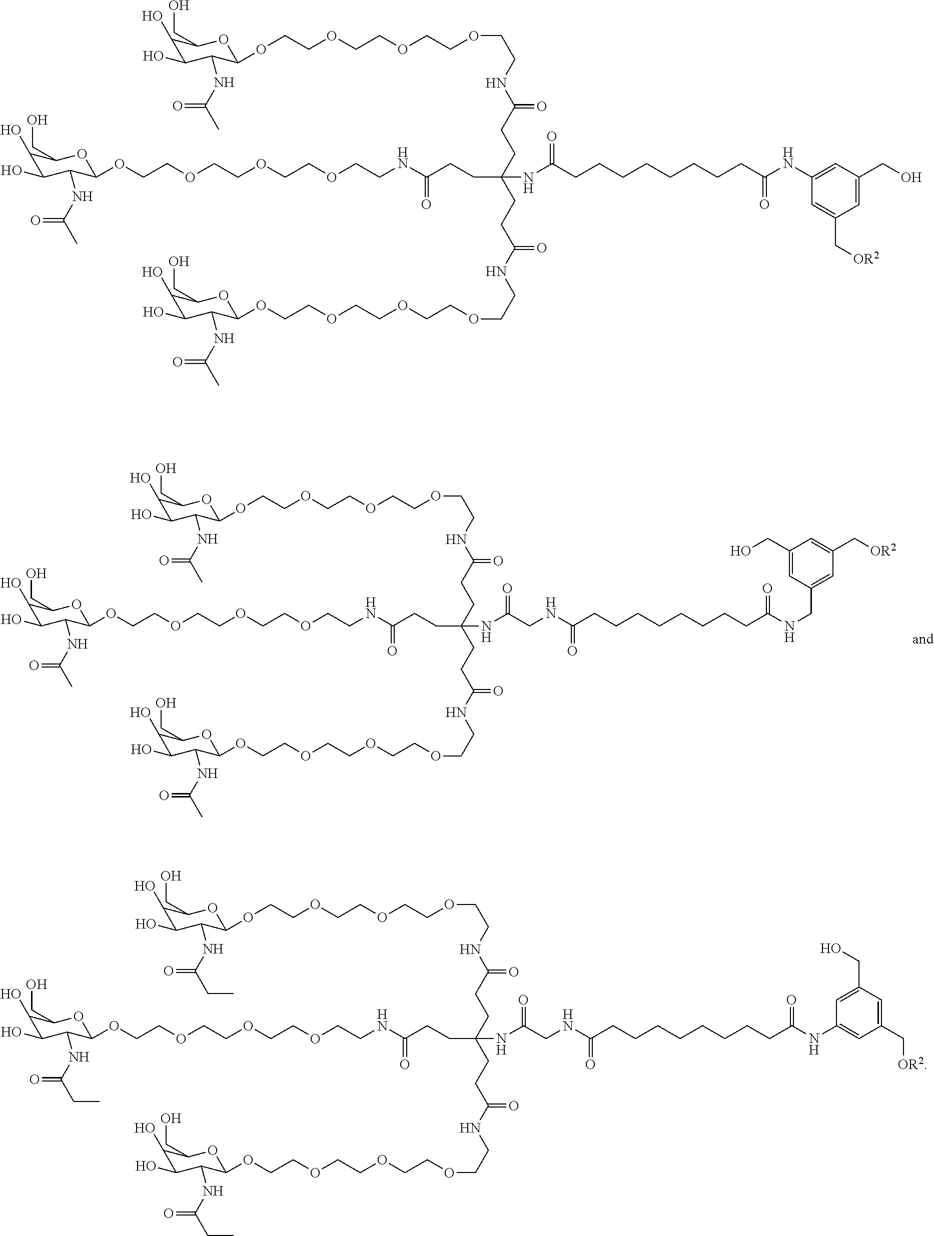

151. The method of any one of claims 1-29, wherein the compound of formula (X) is a compound of formula (I) that is selected from the group consisting of:

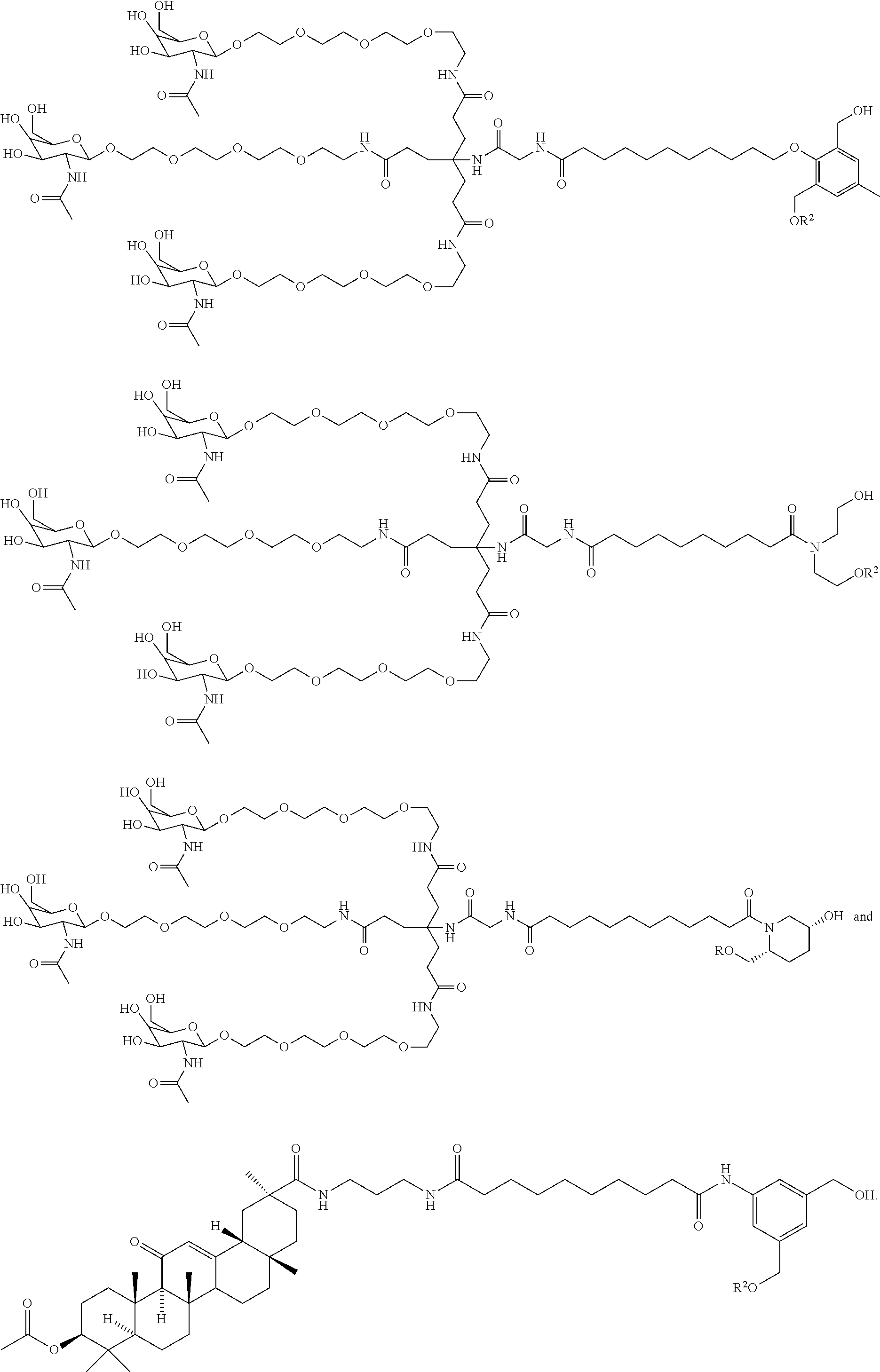

152. The method of any one of claims 1-29, wherein the compound of formula (X) is a compound of formula (I) that is selected from the group consisting of:

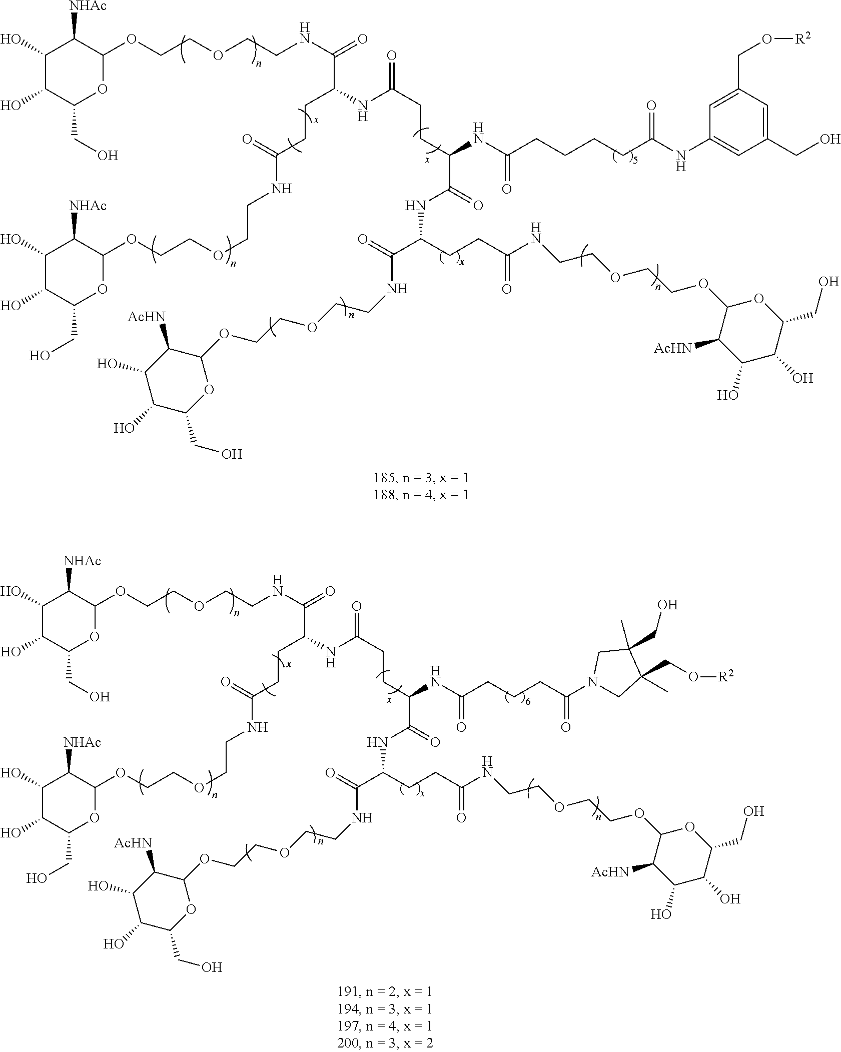

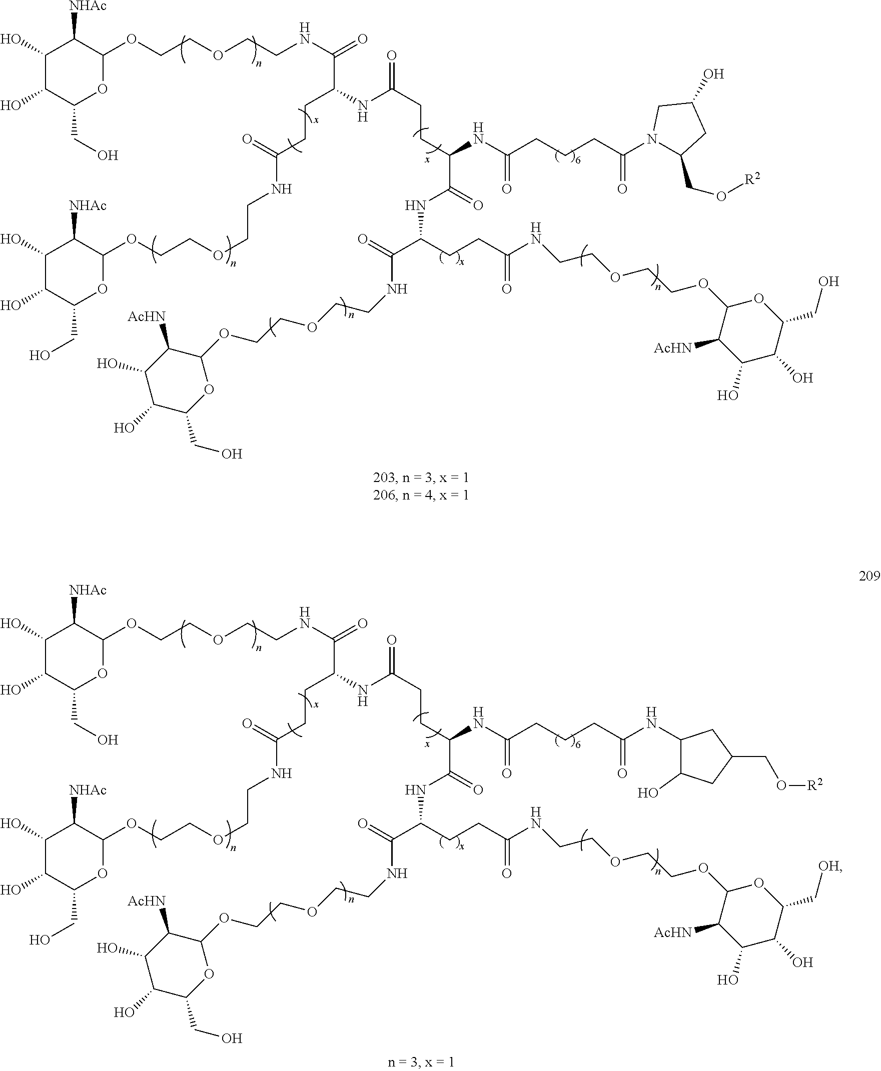

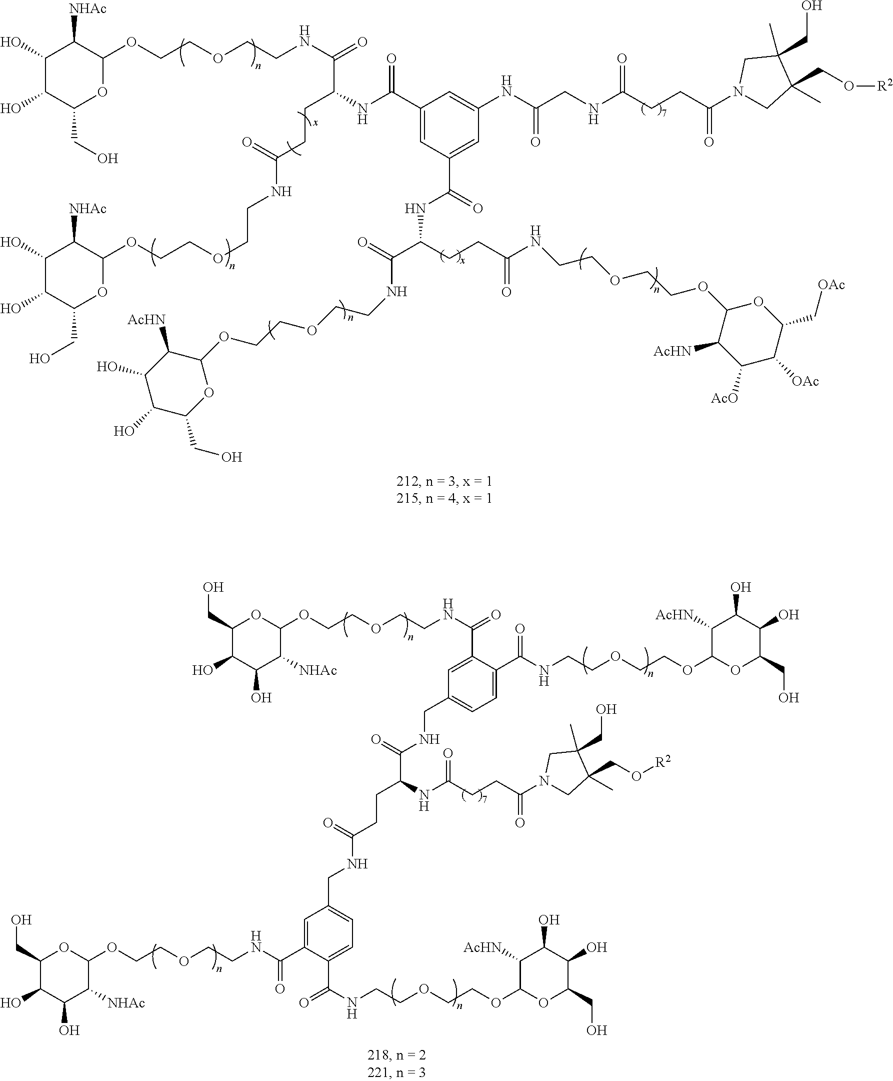

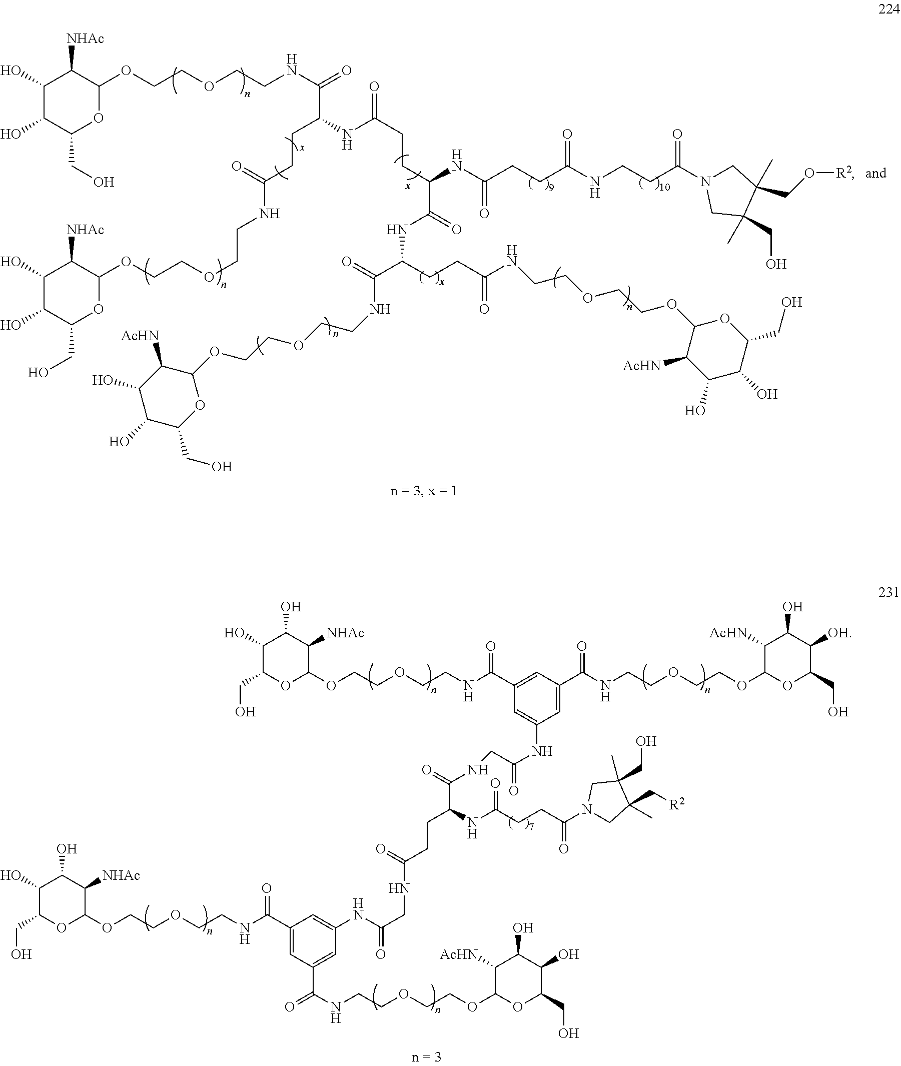

153. The method of any one of claims 1-29, wherein the compound of formula (X) is a compound of formula (I) that is:

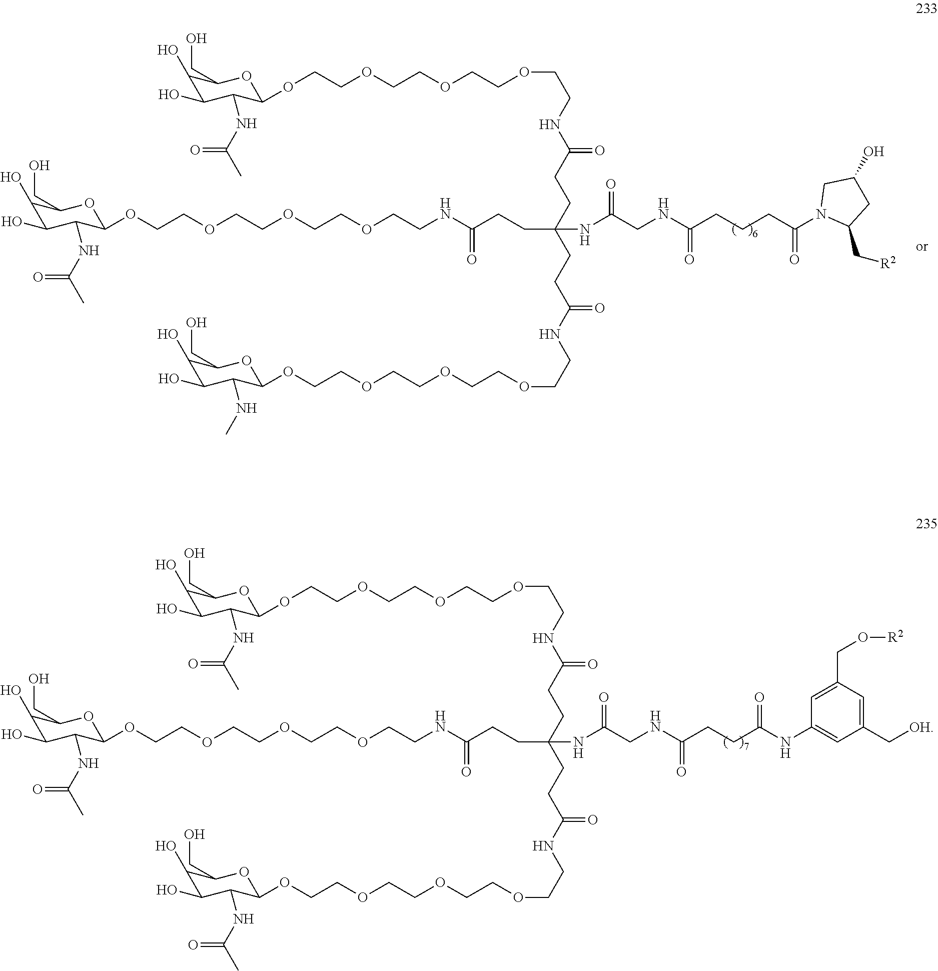

154. The method of any one of claims 1-29, wherein the compound of formula (X) is a compound of formula (I):

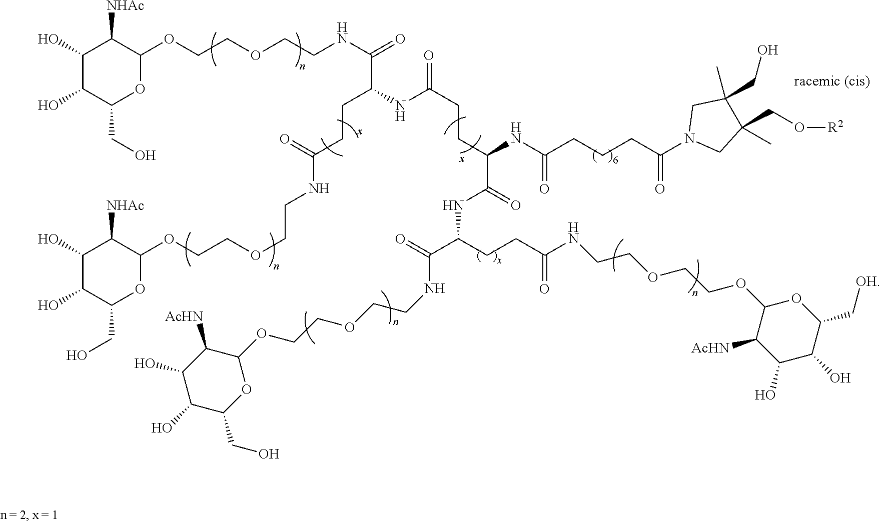

155. The method of any one of claims 1-29, wherein the compound of formula (X) is a compound of formula (I):

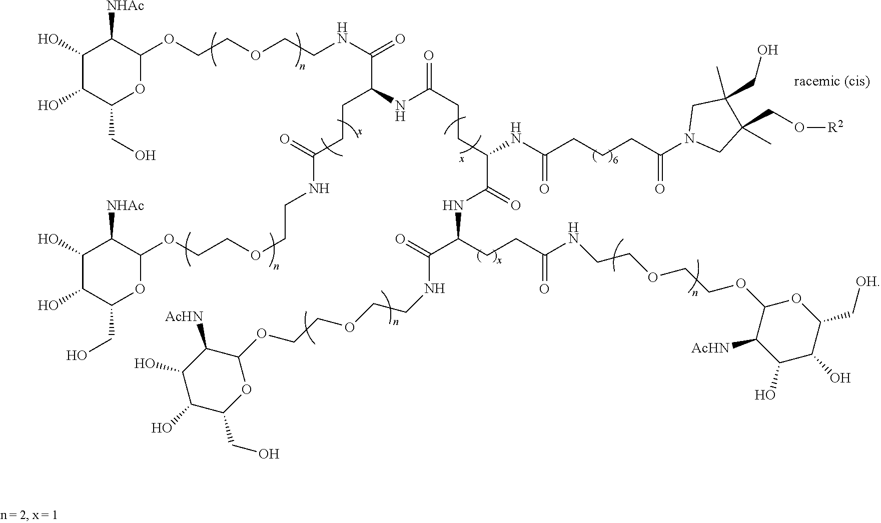

156. The method of any one of claims 1-29, wherein the compound of formula (X) is a compound of formula (I):

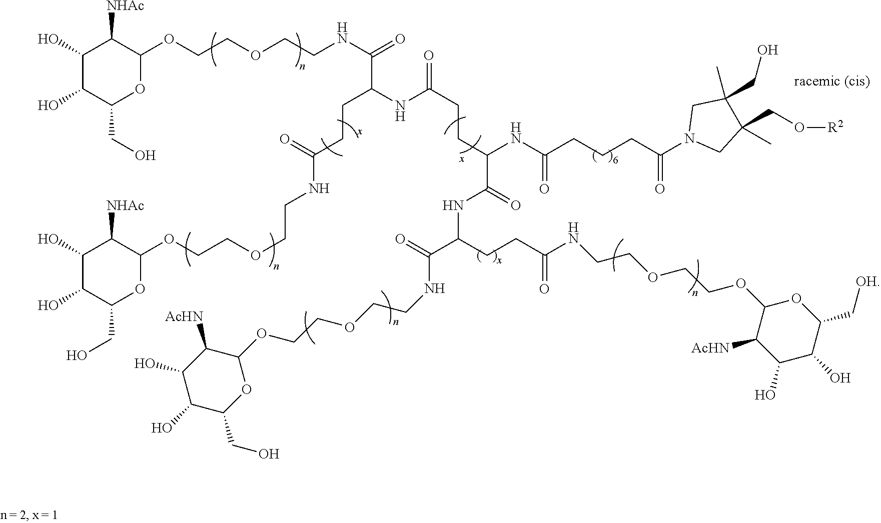

157. The method of any one of claims 1-29, wherein the compound of formula (X) is a compound of formula (I):

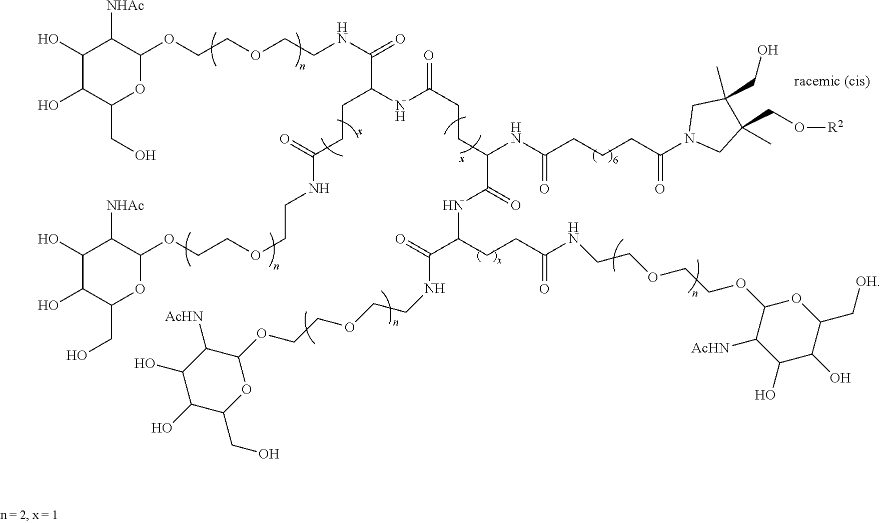

158. The method of any one of claims 1-29, wherein the compound of formula (X) is a compound of formula (I):

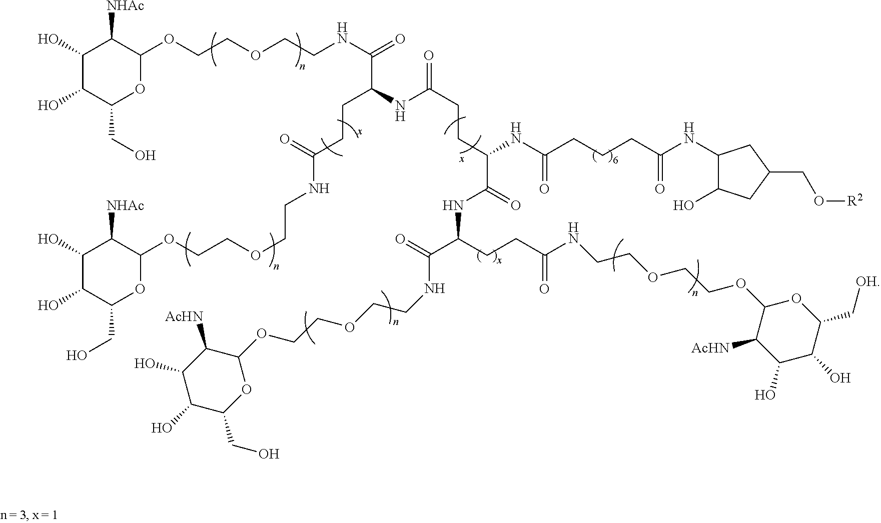

159. The method of any one of claims 1-29, wherein the compound of formula (X) is a compound of formula (I):

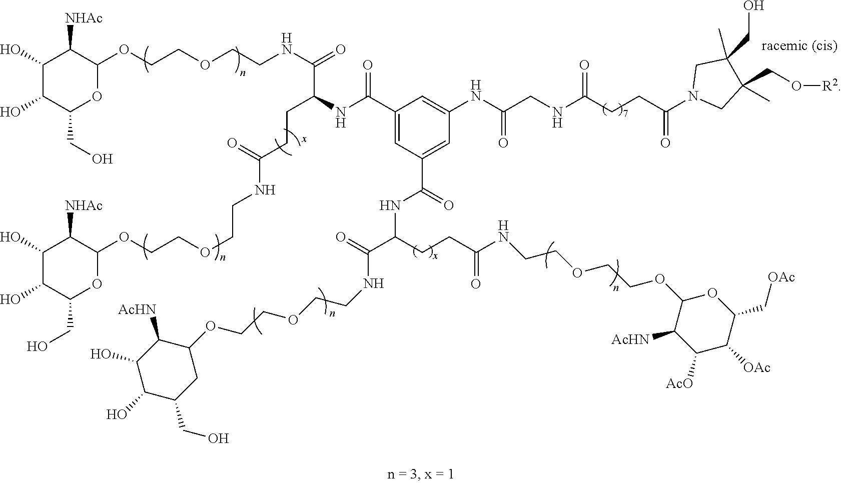

160. The method of any one of claims 1-29, wherein the compound of formula (X) is a compound of formula (I):

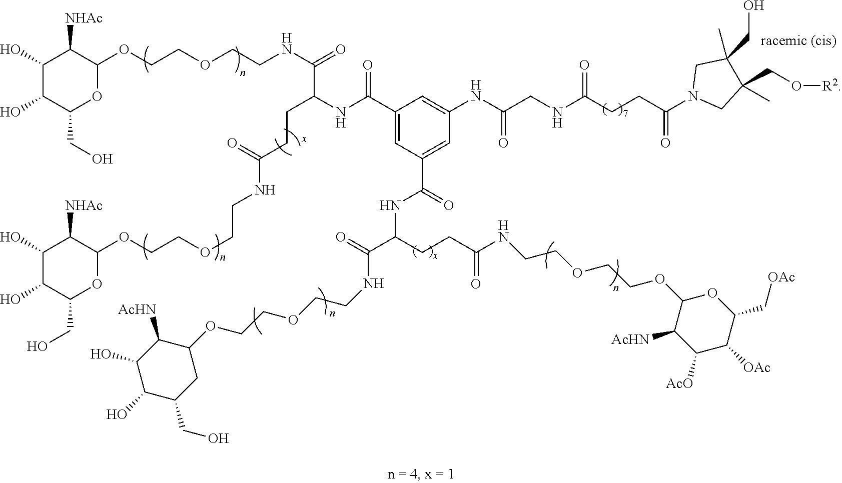

161. The method of any one of claims 1-29, wherein the compound of formula (X) is a compound of formula (I):

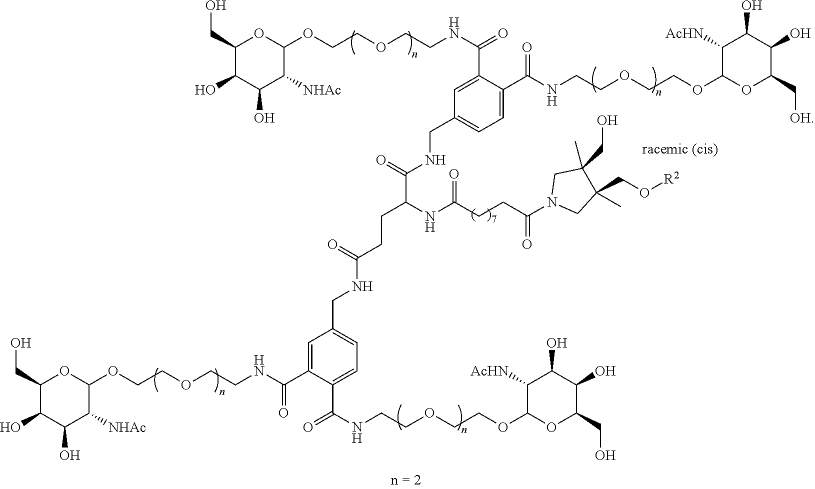

162. The method of any one of claims 1-29, wherein the compound of formula (X) is a compound of formula (I):

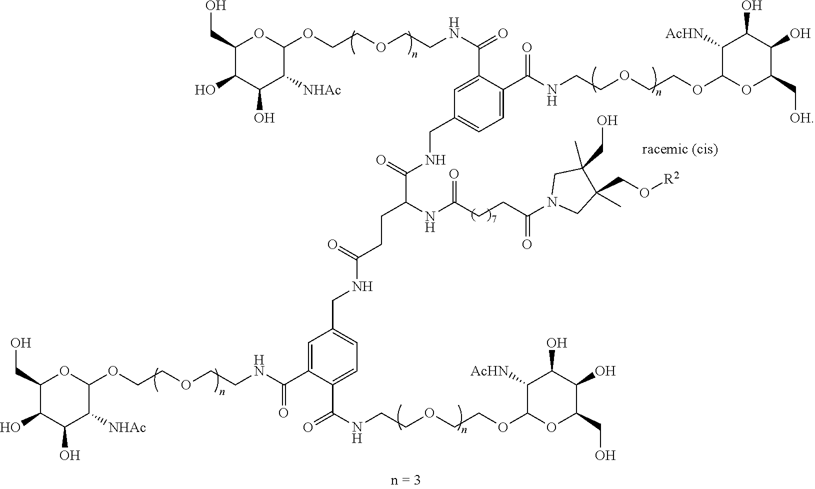

163. The method of any one of claims 1-29, wherein the compound of formula (X) is a compound of formula (I):

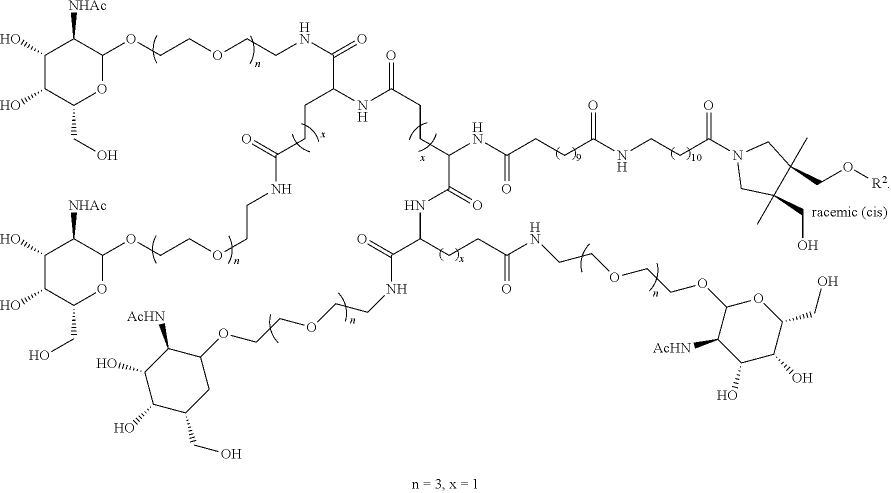

164. The method of any one of claims 1-29, wherein the compound of formula (X) is a compound of formula (I):

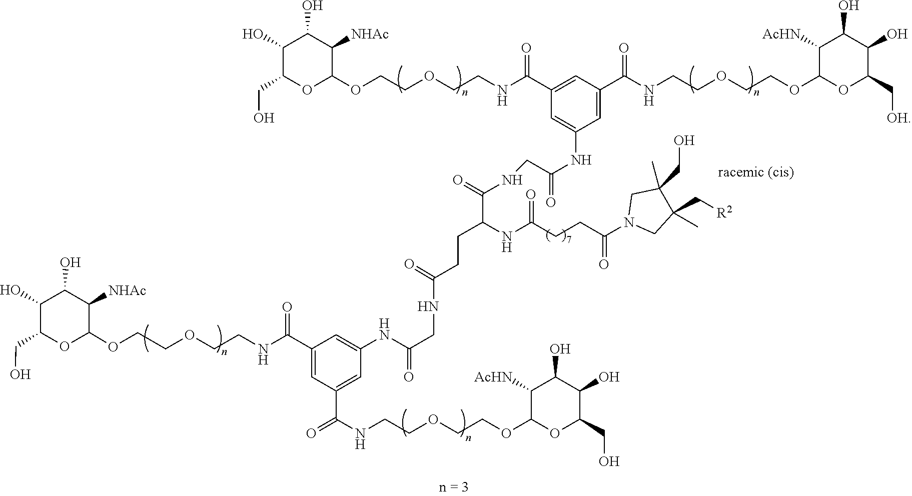

165. The method of any one of claims 1-29, wherein the compound of formula (X) is a compound of formula (I):

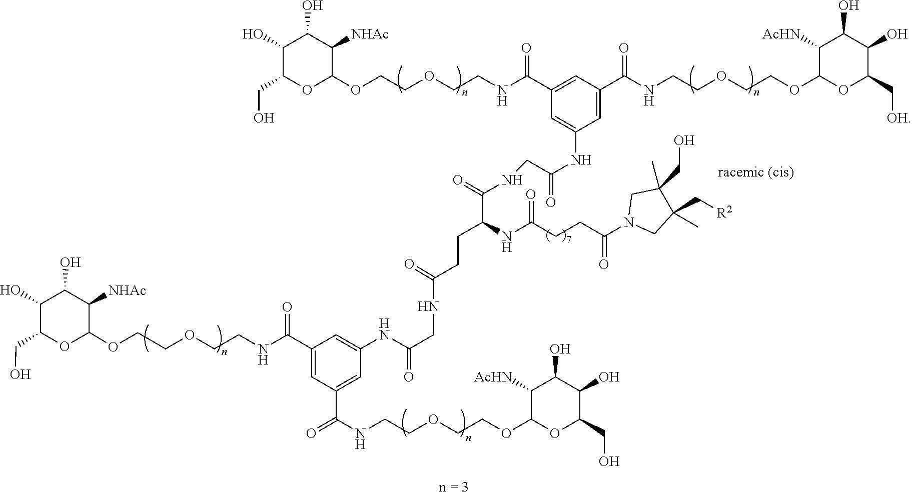

166. The method of any one of claims 1-29, wherein the compound of formula (X) is a compound of formula (I):

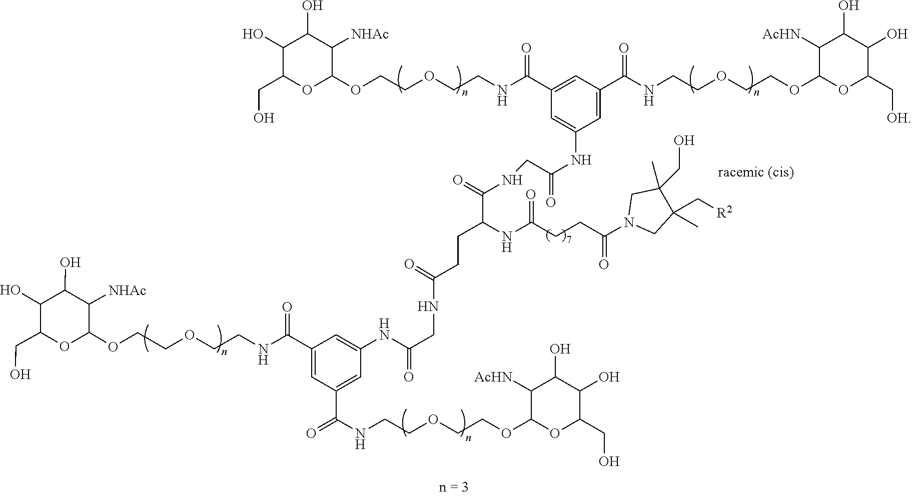

167. The method of any one of claims 1-29, wherein the compound of formula (X) is a compound of formula: 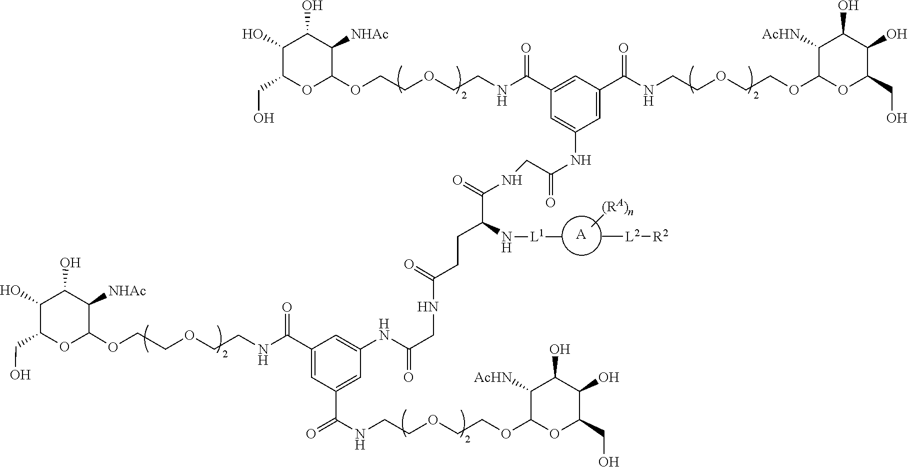
wherein:
L¹ is absent or a linking group;
L² is absent or a linking group;
R² is a nucleic acid;
the ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;
R^{B} is hydrogen, a protecting group, a covalent bond to a solid support, or a bond to a linking group that is bound to a solid support; and
n is 0, 1, 2, 3, 4, 5, 6, 7, 8, 9, or 10.

168. The method of any one of claims 1-29, wherein the compound of formula (X) is a compound of formula: 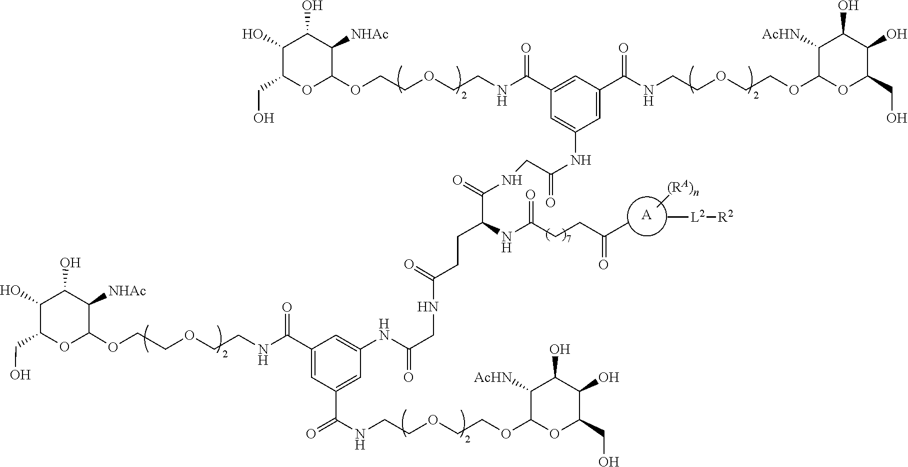
wherein:
L² is absent or a linking group;
R² is a nucleic acid;
the ring A is absent, a 3-20 membered cycloalkyl, a 5-20 membered aryl, a 5-20 membered heteroaryl, or a 3-20 membered heterocycloalkyl;
each R^{A} is independently selected from the group consisting of hydrogen, hydroxy, CN, F, Cl, Br, I, —C₁₋₂ alkyl-OR^{B}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;
R^{B} is hydrogen, a protecting group, a covalent bond to a solid support, or a bond to a linking group that is bound to a solid support; and
n is 0, 1, 2, 3, 4, 5, 6, 7, 8, 9, or 10.

169. The method of any one of claims 1-29, wherein the compound of formula (X) is a compound of formula:

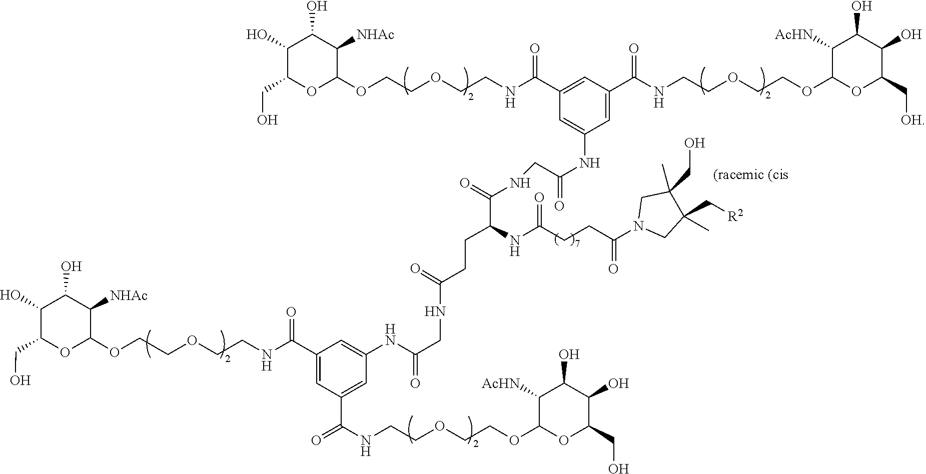

170. The method of any one of claims 1-29, wherein the compound of formula (X) is a compound of formula:

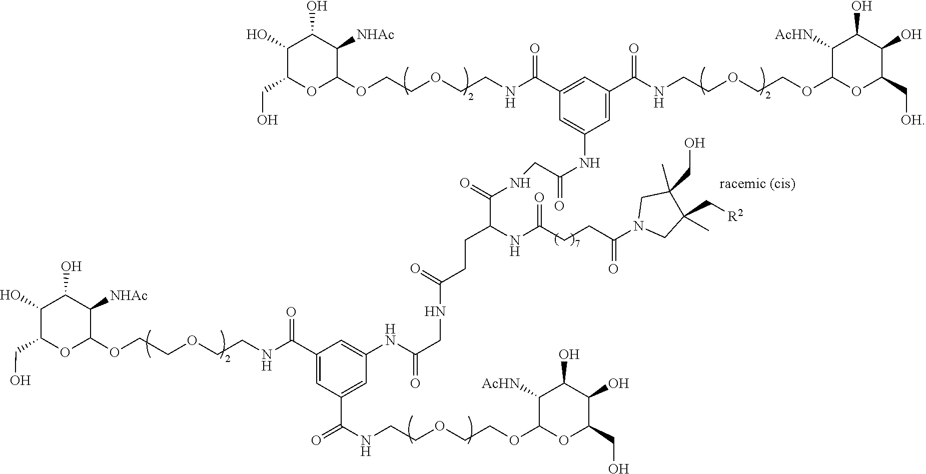

171. The method of any one of claims 1-29, wherein the compound of formula (X) is a compound of formula:

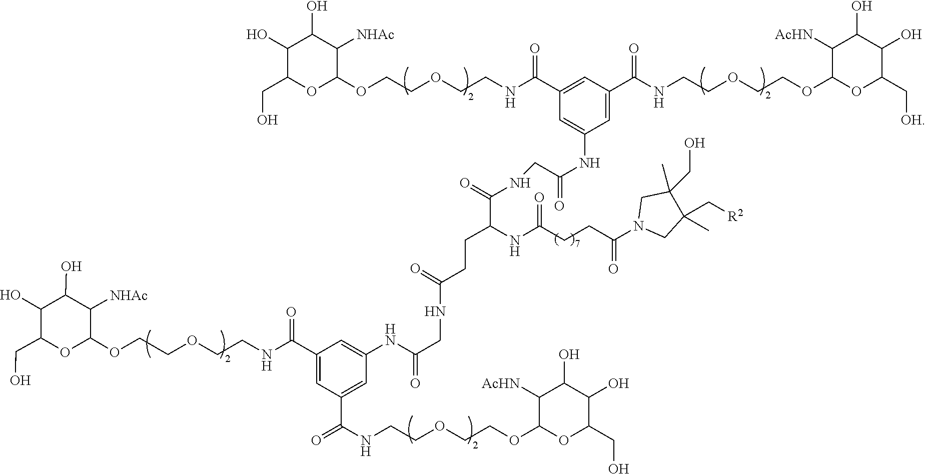

172. The method of any one of claims 1-29, wherein the compound of formula (X) is a compound of formula: 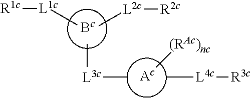
wherein:
R^{1c} is a saccharide;
L^{1c} is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;
B^{c} is a 5-10 membered aryl or a 5-10 membered heteroaryl, which 5-10 membered aryl or 5-10 membered heteroaryl is optionally substituted with one or more groups independently selected from the group consisting of halo, hydroxy, cyano, trifluoromethyl, trifluoromethoxy, (C₁-C₆)alkyl, (C₁-C₆)alkoxy, (C₁-C₆)alkoxycarbonyl, (C₁-C₆)alkanoyloxy, (C₃-C₆)cycloalkyl, and (C₃-C₆)cycloalkyl(C₁-C₆)alkyl
L^{2c} is a divalent, branched or unbranched, saturated or unsaturated, hydrocarbon chain, having from 0 to 20 carbon atoms, wherein one or more of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo;
R^{2c} is a saccharide;
L^{3c} is absent or a linking group;
A^{c} is a 3-20 membered cycloalkyl, a 5-20 membered aryl, a 5-20 membered heteroaryl, or a 3-20 membered heterocycloalkyl;
each R^{Ac} is independently selected from the group consisting of hydrogen, hydroxy, CN, F, Cl, Br, I, —C₁₋₂ alkyl-OR^{a}, C₁₋₁₀ alkyl C₂₋₁₀ alkenyl, and C₂₋₁₀ alkynyl; wherein the C₁₋₁₀ alkyl C₂₋₁₀ alkenyl, and C₂₋₁₀ alkynyl are optionally substituted with one or more groups independently selected from halo, hydroxy, and C₁₋₃ alkoxy;
nc is 0, 1, 2, 3, 4, 5, 6, 7, 8, 9, or 10;
L^{4c} is absent or a linking group;
R^{3c} is a nucleic acid;
R^{ac} is hydrogen; and
L^{5c} is a linking group.

173. The method of claim 172, wherein B^{c} is a 5-10 membered aryl.

174. The method of claim 172, wherein B^{c} is naphthyl or phenyl.

175. The method of claim 172, wherein B^{c} is phenyl.

176. The method of claim 172, wherein the group:

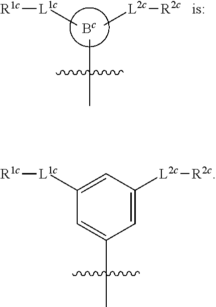

177. The method of claim 172, wherein B^{c} is a 5-10 membered heteroaryl.

178. The method of claim 172, wherein B^{c} is pyridyl, pyrimidyl, quinolyl, isoquinolyl, imidazolyl, thiazolyl, oxadiazolyl or oxazolyl.

179. The method of claim 172, wherein the group:

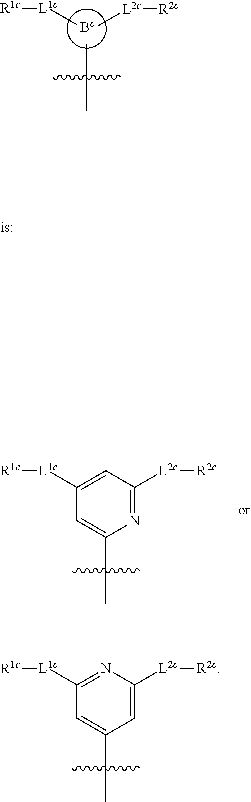

180. The method of claim 172, wherein the group:

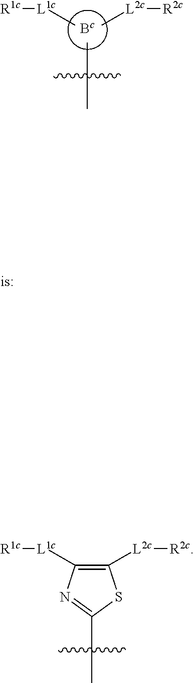

181. The method of any one of claims 172-180, wherein L^{1c} is a divalent, unbranched, saturated hydrocarbon chain, having from 0 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo.

182. The method of any one of claims 172-180, wherein L^{1c} is a divalent, unbranched, saturated hydrocarbon chain, having from 0 to 12 carbon atoms, wherein one or more (e.g. 1, 2, 3, or 4) of the carbon atoms in the hydrocarbon chain is optionally replaced by —O—, —NR^{X}—C(═O)—, or —C(═O)—NR^{X}—, and wherein R^{X} is hydrogen or (C₁-C₆)alkyl.

183. The method of any one of claims 172-180, wherein L^{1c} is:
—C(═O)N(H)—CH₂CH₂OCH₂CH₂OCH₂CH₂—,
—C(═O)N(H)—CH₂CH₂OCH₂CH₂OCH₂CH₂OCH₂CH₂—,
—C(═O)N(CH₃)—CH₂CH₂OCH₂CH₂OCH₂CH₂—, or
—C(═O)N(CH₃)—CH₂CH₂OCH₂CH₂OCH₂CH₂OCH₂CH₂—.

184. The method of any one of claims 172-183, wherein L^{2c} is a divalent, unbranched, saturated hydrocarbon chain, having from 0 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more substituents selected from oxo (═O) and halo.

185. The method of any one of claims 172-183, wherein L^{2c} is a divalent, unbranched, saturated hydrocarbon chain, having from 0 to 12 carbon atoms, wherein one or more (e.g. 1, 2, 3, or 4) of the carbon atoms in the hydrocarbon chain is optionally replaced by —O—, —NR^{X}—C(═O)—, or —C(═O)—NR^{X}—, and wherein R^{X} is hydrogen or (C₁-C₆)alkyl.

186. The method of any one of claims 172-183, wherein L^{2c} is:
—C(═O)N(H)—CH₂CH₂OCH₂CH₂OCH₂CH₂—,
—C(═O)N(H)—CH₂CH₂OCH₂CH₂OCH₂CH₂OCH₂CH₂—,
—C(═O)N(CH₃)—CH₂CH₂OCH₂CH₂OCH₂CH₂—, or
—C(═O)N(CH₃)—CH₂CH₂OCH₂CH₂OCH₂CH₂OCH₂CH₂—.

187. The method of any one of claims 172-186, wherein R^{1c} is: 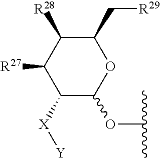
wherein:
X is NR²⁰ and Y is selected from —(C═O)R²¹, —SO₂R²², and —(C═O)NR²³R²⁴; or X is —(C═O)— and Y is NR²⁵SR²⁶; or X is —NR³⁷R³⁸ and Y is absent
R²⁰ is hydrogen or (C₁-C₄)alkyl;
R²¹, R²², R²³, R²⁴, R²⁵ and R²⁶ are each independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo, (C₁-C₄)alkyl, and (C₁-C₄)alkoxy;
R²⁷ is —OH, —NR²⁵R²⁶ or —F;
R²⁸ is —OH, —NR²⁵R²⁶ or —F;
R²⁹ is —OH, —NR²⁵R²⁶, —F, —N₃, —NR³⁵R³⁶, or 5 membered heterocycle that is optionally substituted with one or more groups independently selected from the group consisting of halo, hydroxyl, carboxyl, amino, (C₁-C₄)alkyl, aryl, and (C₁-C₄)alkoxy, wherein any (C₁-C₄)alkyl, and (C₁-C₄)alkoxy is optionally substituted with one or more groups independently selected from the group consisting of halo, and wherein any aryl is optionally substituted with one or more groups independently selected from the group consisting of halo, hydroxyl, nitro, cyano, amino, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy;
each R³⁵ and R³⁶ is independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo and (C₁-C₄)alkoxy; or R³ and R⁶ taken together with the nitrogen to which they are attached form a 5-6 membered heteroaryl ring, which heteroaryl ring is optionally substituted with one or more groups independently selected from the group consisting of (C₁-C₈)alkyl, (C₁-C₈)alkoxy, aryl, and (C₃-C₆)cycloalkyl, wherein any aryl, and (C₃-C₆)cycloalkyl is optionally substituted with one or more groups R³⁹;
each R³⁷ and R³⁸ is independently selected from the group consisting of hydrogen, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy; or R³⁷ and R³⁸ taken together with the nitrogen to which they are attached form a 5-8 membered heterocycle that is optionally substituted with one or more groups independently selected from the group consisting of halo, hydroxyl, carboxyl, amino, oxo (═O), (C₁-C₄)alkyl, and (C₁-C₄)alkoxy, wherein any (C₁-C₄)alkyl, and (C₁-C₄)alkoxy is optionally substituted with one or more groups independently selected from halo; and
each R³⁹ is independently selected from the group consisting of (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from halo.

188. The method of any one of claims 172-186, wherein R^{1c} is:

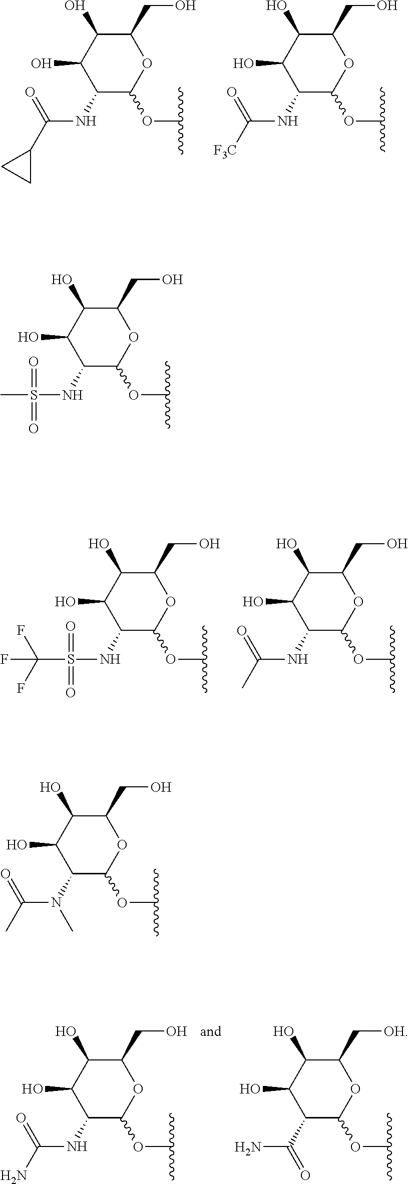

189. The method of any one of claims 172-186, wherein R^{1c} is:

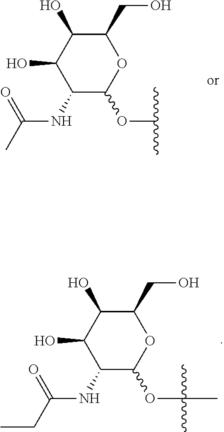 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

190. The method of any one of claims 172-186, wherein R^{1c} is:

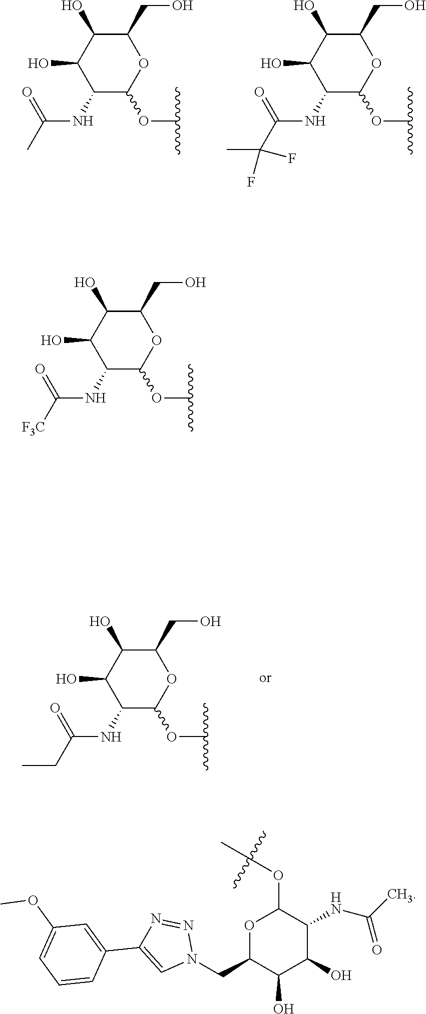 [SMILES: *OC1O[C@H](CO)[C@H](O)[C@H](O)[C@H]1NC(C)=O ; *OC1O[C@H](CO)[C@H](O)[C@H](O)[C@H]1NC(=O)C(C)(F)F ; *OC1O[C@H](CO)[C@H](O)[C@H](O)[C@H]1NC(=O)C(F)(F)F ; *OC1O[C@H](CO)[C@H](O)[C@H](O)[C@H]1NC(=O)CC ; *OC1O[C@H](Cn2cc(-c3cccc(OC)c3)nn2)[C@H](O)[C@H](O)[C@H]1NC(C)=O]

191. The method of any one of claims 172-186, wherein R^{1c} is:

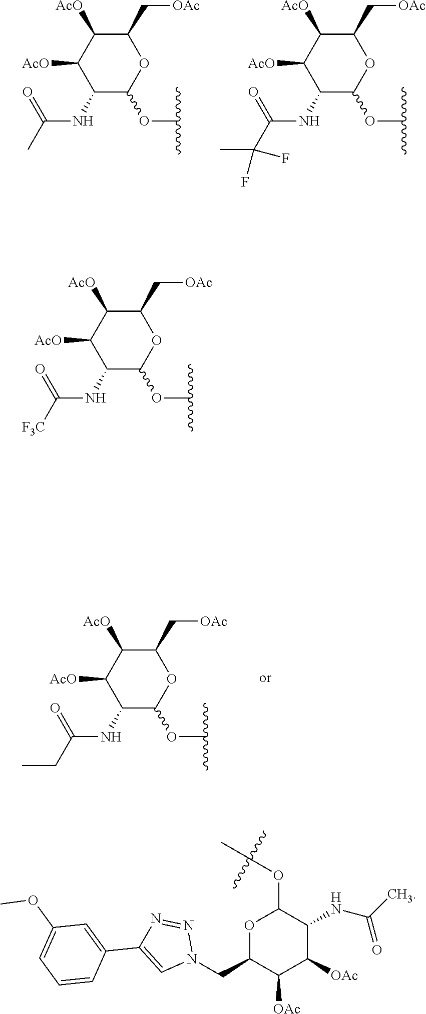 [SMILES: *OC1O[C@H](COC(C)=O)[C@H](OC(C)=O)[C@H](OC(C)=O)[C@H]1NC(C)=O ; *OC1O[C@H](COC(C)=O)[C@H](OC(C)=O)[C@H](OC(C)=O)[C@H]1NC(=O)C(C)(F)F ; *OC1O[C@H](COC(C)=O)[C@H](OC(C)=O)[C@H](OC(C)=O)[C@H]1NC(=O)C(F)(F)F ; *OC1O[C@H](COC(C)=O)[C@H](OC(C)=O)[C@H](OC(C)=O)[C@H]1NC(=O)CC ; *OC1O[C@H](Cn2cc(-c3cccc(OC)c3)nn2)[C@H](OC(C)=O)[C@H](OC(C)=O)[C@H]1NC(C)=O]

192. The method of any one of claims 172-186, wherein R^{2c} is: 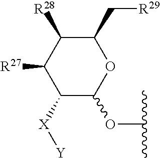
wherein:
X is NR²⁰ and Y is selected from —(C═O)R²¹, —SO₂R²², and —(C═O)NR²³R²⁴; or X is —(C═O)— and Y is NR²⁵R²⁶; or X is —NR³⁷R³⁸ and Y is absent
R²⁰ is hydrogen or (C₁-C₄)alkyl;
R²¹, R²², R²³, R²⁴, R²⁵ and R²⁶ are each independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo, (C₁-C₄)alkyl, and (C₁-C₄)alkoxy;
R²⁷ is —OH, —NR²⁵R²⁶ or —F;
R²⁸ is —OH, —NR²⁵R²⁶ or —F;
R²⁹ is —OH, —NR²⁵R²⁶, —F, —N₃, —NR³⁵R³⁶, or 5 membered heterocycle that is optionally substituted with one or more groups independently selected from the group consisting of halo, hydroxyl, carboxyl, amino, (C₁-C₄)alkyl, aryl, and (C₁-C₄)alkoxy, wherein any (C₁-C₄)alkyl, and (C₁-C₄)alkoxy is optionally substituted with one or more groups independently selected from the group consisting of halo, and wherein any aryl is optionally substituted with one or more groups independently selected from the group consisting of halo, hydroxyl, nitro, cyano, amino, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy;
each R³⁵ and R³⁶ is independently selected from the group consisting of hydrogen, (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from the group consisting of halo and (C₁-C₄)alkoxy; or R³⁵ and R³⁶ taken together with the nitrogen to which they are attached form a 5-6 membered heteroaryl ring, which heteroaryl ring is optionally substituted with one or more groups independently selected from the group consisting of (C₁-C₈)alkyl, (C₁-C₈)alkoxy, aryl, and (C₃-C₆)cycloalkyl, wherein any aryl, and (C₃-C₆)cycloalkyl is optionally substituted with one or more groups R³⁹;
each R³⁷ and R³⁸ is independently selected from the group consisting of hydrogen, (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl, wherein any (C₁-C₈)alkyl, (C₁-C₈)alkoxy, (C₁-C₈)alkanoyl, (C₁-C₈)alkoxycarbonyl, (C₁-C₈)alkanoyloxy, and (C₃-C₆)cycloalkyl is optionally substituted with one or more groups independently selected from the group consisting of halo, (C₁-C₄)alkyl, and (C₁-C₄)alkoxy; or R³⁷ and R³⁸ taken together with the nitrogen to which they are attached form a 5-8 membered heterocycle that is optionally substituted with one or more groups independently selected from the group consisting of halo, hydroxyl, carboxyl, amino, oxo (═O), (C₁-C₄)alkyl, and (C₁-C₄)alkoxy, wherein any (C₁-C₄)alkyl, and (C₁-C₄)alkoxy is optionally substituted with one or more groups independently selected from halo; and
each R³⁹ is independently selected from the group consisting of (C₁-C₈)alkyl, (C₁-C₈)alkoxy and (C₃-C₆)cycloalkyl, wherein any (C₁-C₈)alkyl, (C₁-C₈)alkoxy and (C₃-C₆)cycloalkyl is optionally substituted with one or more groups independently selected from halo.

193. The method of any one of claims 172-186, wherein R^{2c} is:

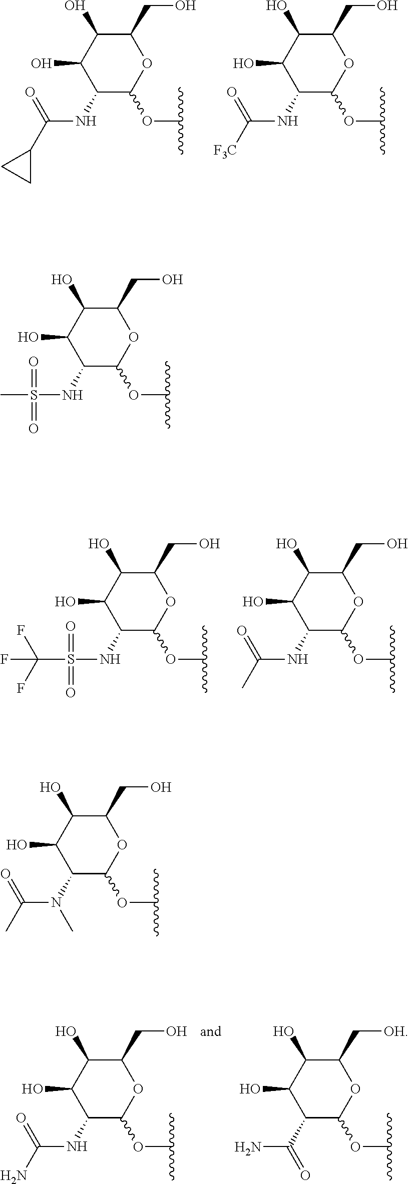

194. The method of any one of claims 172-186, wherein R^{2c} is:

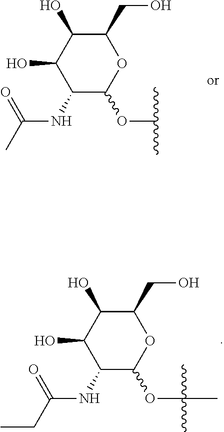 [SMILES: *OC1O[C@H](CO)[C@H](O)[C@H](O)[C@H]1NC(C)=O ; *OC1O[C@H](CO)[C@H](O)[C@H](O)[C@H]1NC(=O)CC]

195. The method of an one of claims 172-186 wherein R^{2c} is:

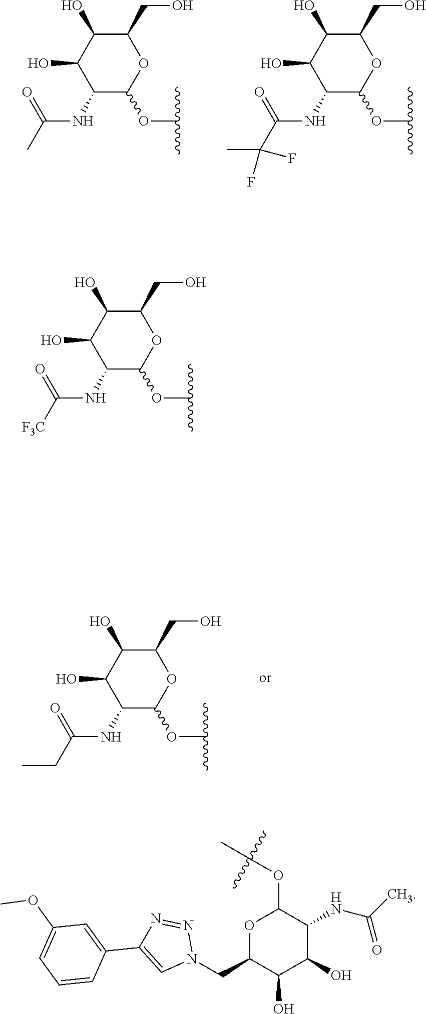 [SMILES: *OC1O[C@H](CO)[C@H](O)[C@H](O)[C@H]1NC(C)=O ; *OC1O[C@H](CO)[C@H](O)[C@H](O)[C@H]1NC(=O)C(C)(F)F ; *OC1O[C@H](CO)[C@H](O)[C@H](O)[C@H]1NC(=O)C(F)(F)F ; *OC1O[C@H](CO)[C@H](O)[C@H](O)[C@H]1NC(=O)CC ; *OC1O[C@H](Cn2cc(-c3cccc(OC)c3)nn2)[C@H](O)[C@H](O)[C@H]1NC(C)=O]

196. The method of any one of claims 172-186, wherein R^{2c} is

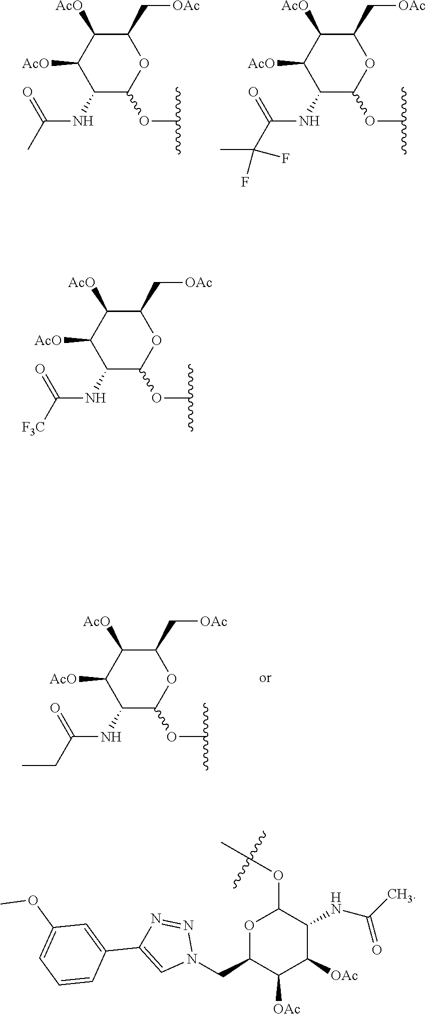 [SMILES: *OC1O[C@H](COC(C)=O)[C@H](OC(C)=O)[C@H](OC(C)=O)[C@H]1NC(C)=O ; *OC1O[C@H](COC(C)=O)[C@H](OC(C)=O)[C@H](OC(C)=O)[C@H]1NC(=O)C(C)(F)F ; *OC1O[C@H](COC(C)=O)[C@H](OC(C)=O)[C@H](OC(C)=O)[C@H]1NC(=O)C(F)(F)F ; *OC1O[C@H](COC(C)=O)[C@H](OC(C)=O)[C@H](OC(C)=O)[C@H]1NC(=O)CC ; *OC1O[C@H](Cn2cc(-c3cccc(OC)c3)nn2)[C@H](OC(C)=O)[C@H](OC(C)=O)[C@H]1NC(C)=O]

197. The method of any one of claims 172-196, wherein L^{3c} is a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

198. The method of any one of claims 172-196, wherein L^{3c} is a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

199. The method of any one of claims 184-196, wherein L^{3c} is a divalent, branched or unbranched, saturated or unsaturated, hydrocarbon chain, having from 1 to 30 carbon atoms, wherein one or more of the carbon atoms is optionally replaced by —O—, —NR^{X}—, —NR^{X}—(=O)—, —C(═O)—NR^{X}— or —S—, and wherein R^{X} is hydrogen or (C₁-C₆)alkyl, and wherein the hydrocarbon chain, is optionally substituted with one or more halo or oxo (═O).

200. The method of any one of claims 172-196, wherein L^{3c} is:

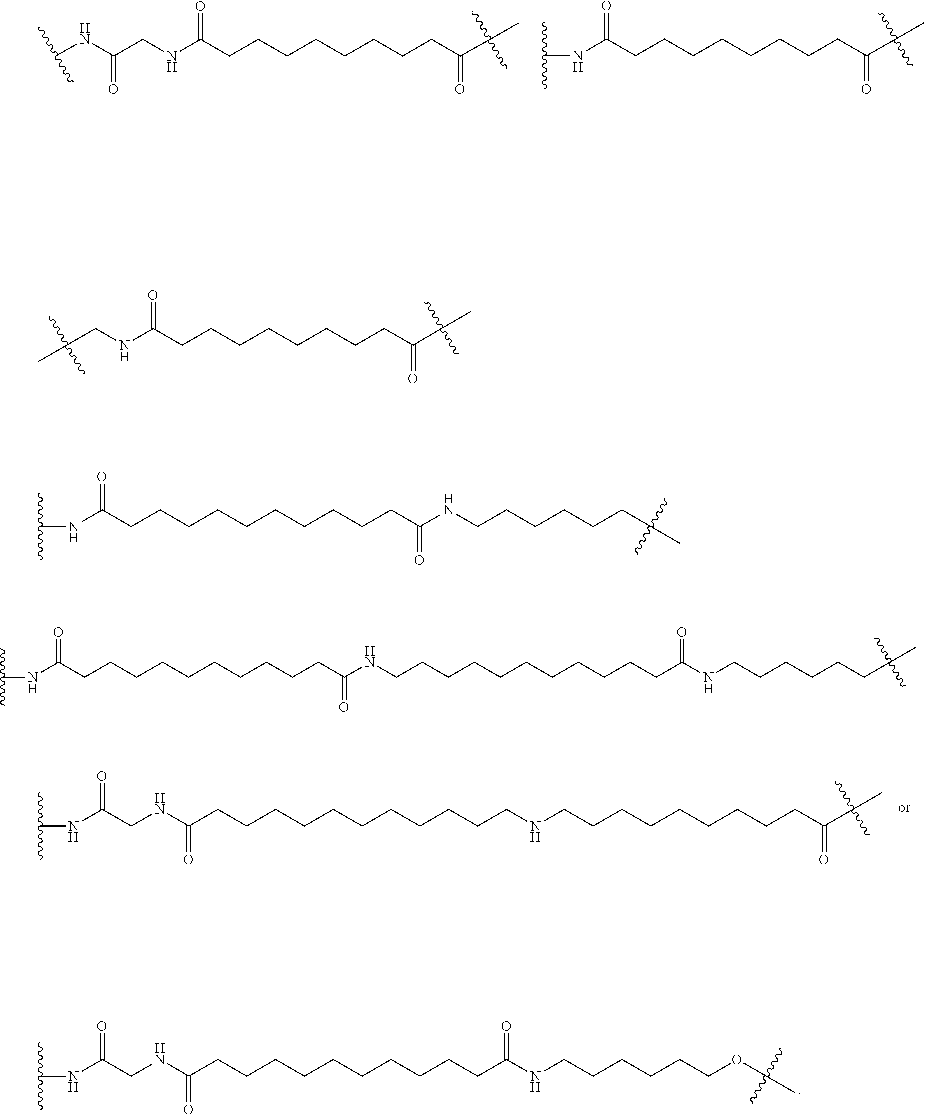

201. The method of any one of claims 172-196, wherein L^{3c} is connected to B through —NH—, —O—, —S—, —(C═O)—, —(C═O)—NH—, —NH—(C═O)—, —(C═O)—O—, —NH—(C═O)—NH—, or —NH—(SO₂)—.

202. The method of any one of claims 172-201, wherein L^{4c} is a divalent, branched or unbranched, saturated or unsaturated, hydrocarbon chain, having from 0 to 50 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

203. The method of any one of claims 172-201, wherein L^{4c} is a divalent, branched or unbranched, saturated or unsaturated, hydrocarbon chain, having from 1 to 20 carbon atoms, wherein one or more (e.g. 1, 2, 3, or 4) of the carbon atoms in the hydrocarbon chain is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more (e.g. 1, 2, 3, or 4) substituents selected from (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

204. The method of any one of claims 172-201, wherein L^{4c} is a divalent, branched or unbranched, saturated or unsaturated, hydrocarbon chain, having from 1 to 30 carbon atoms, wherein one or more of the carbon atoms is optionally replaced by —O—, —NR^{X}—, —NR^{X}—C(═O)—, —C(═O)—NR^{X}— or —S—, and wherein R^{X} is hydrogen or (C₁-C₆)alkyl, and wherein the hydrocarbon chain, is optionally substituted with one or more halo or oxo (═O).

205. The method of claim 172, wherein the group: 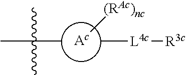
is selected from the group consisting of: 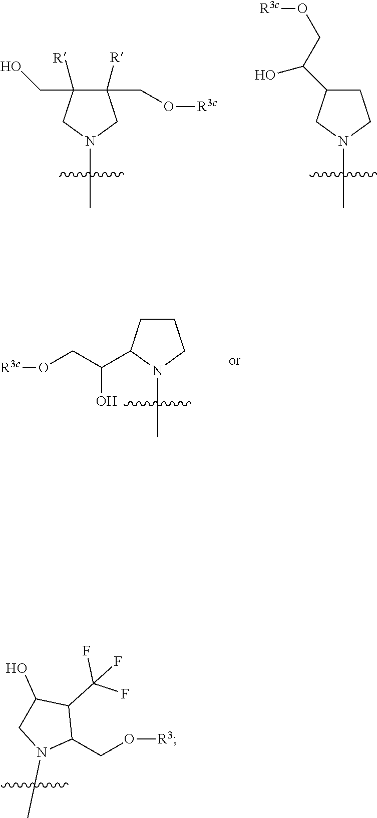
wherein
each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl.

206. The method of claim 172, wherein the group: 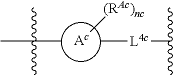
is selected from the group consisting of: 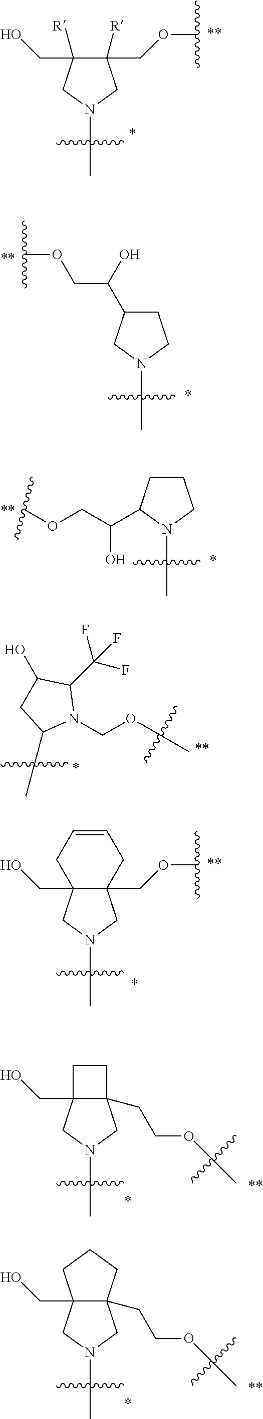 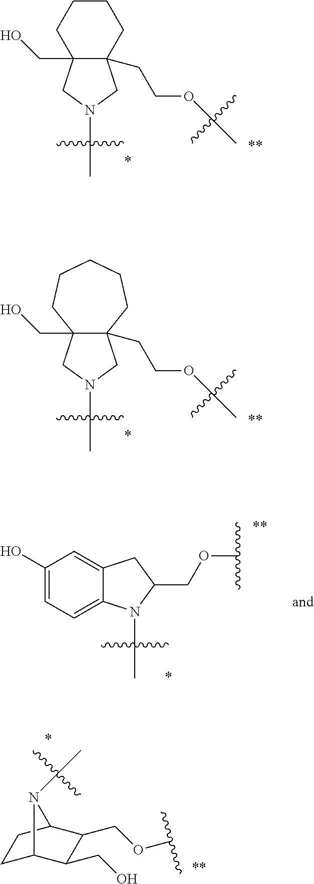
wherein:
each R′ is independently C₁₋₉ alkyl, C₂₋₉ alkenyl or C₂₋₉ alkynyl; wherein the C₁₋₉ alkyl, C₂₋₉ alkenyl or C₂₋₉ alkynyl are optionally substituted with halo or hydroxyl;
the valence marked with * is attached to L^{3c}; and
the valence marked with ** is attached to R^{3c}.

207. The method of claim 172, wherein the group:

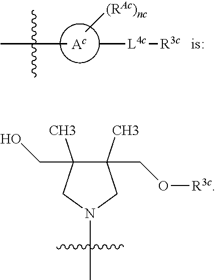

208. The method of any one of claims 172-204, wherein L is attached to R^{3c} through —O—.

209. The method of any one of claims 172-204, wherein R^{3c} is attached to the reminder of the conjugate through the oxygen of a phosphate of the nucleic acid molecule.

210. The method of any one of claims 172-204, wherein R^{3c} is attached to the reminder of the conjugate through the oxygen of a phosphate at the 5′-end of a sense or the antisense strand.

211. The method of any one of claims 172-204, wherein R^{3c} is attached to the reminder of the conjugate through the oxygen of a phosphate at the 3′-end of a sense or the antisense strand.

212. The method of any one of claims 172-204, wherein R^{3c} is attached to the reminder of the conjugate through the oxygen of a phosphate at the 3′-end of a sense strand.

213. The method of any one of claims 1-29, wherein the compound of formula (X) is selected from the group consisting of:

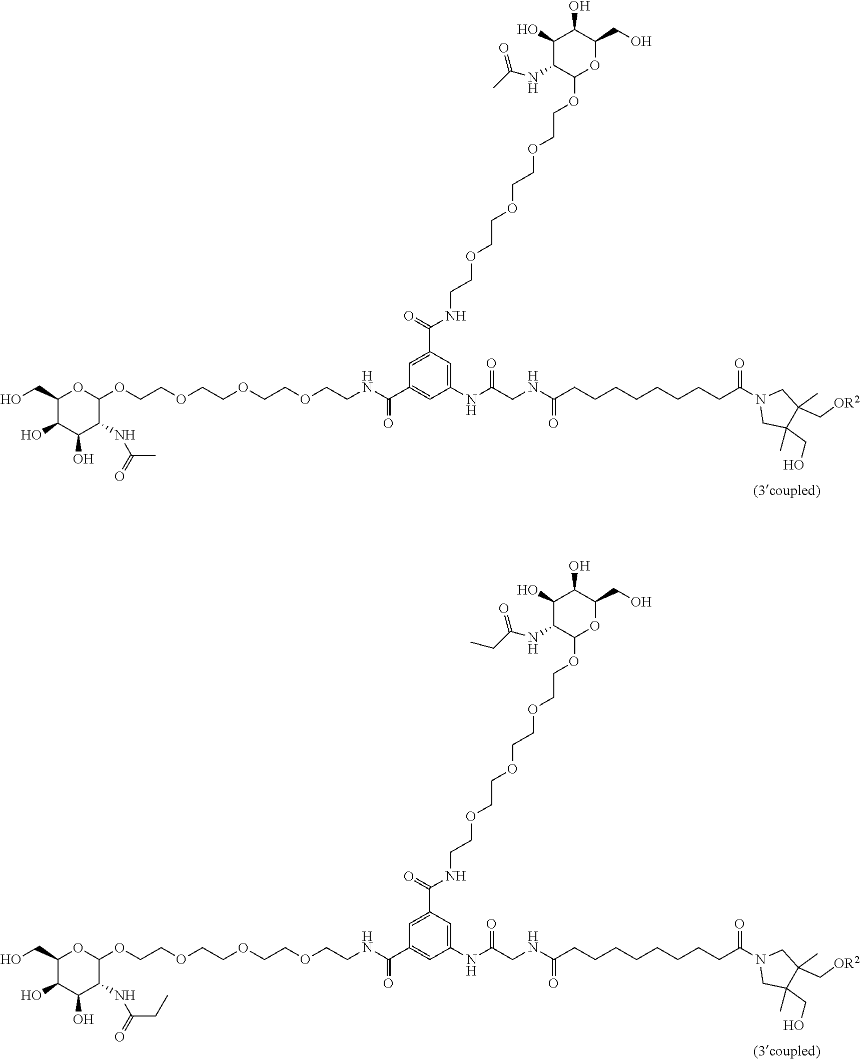

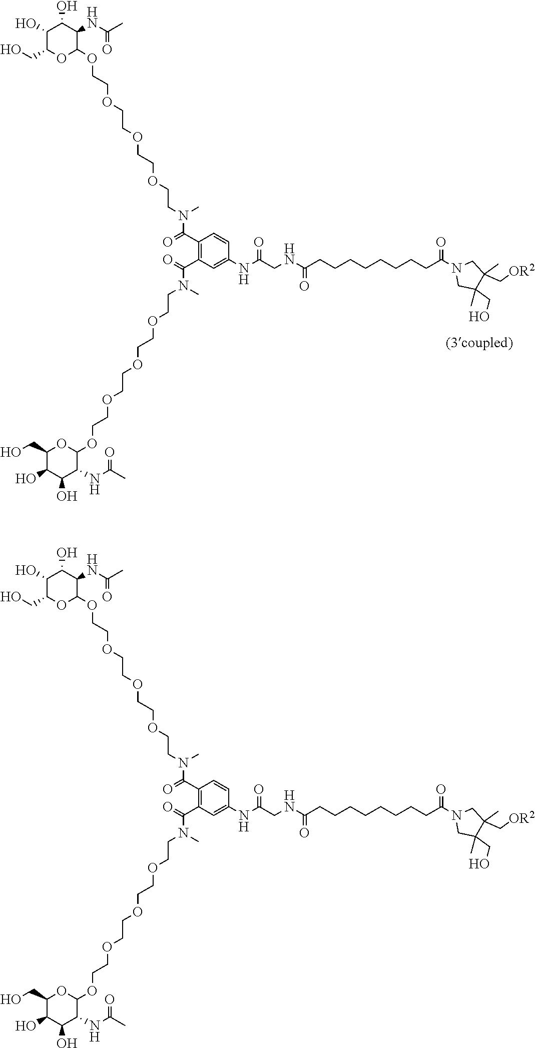

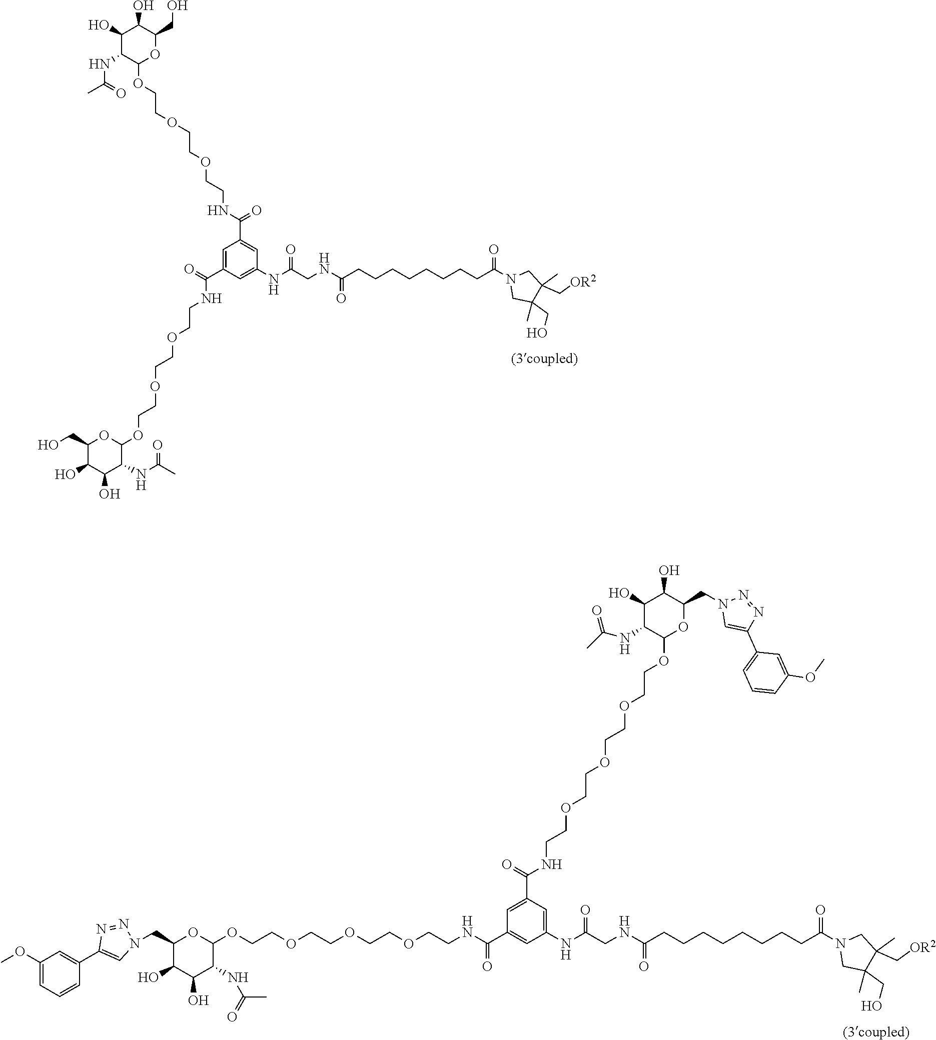

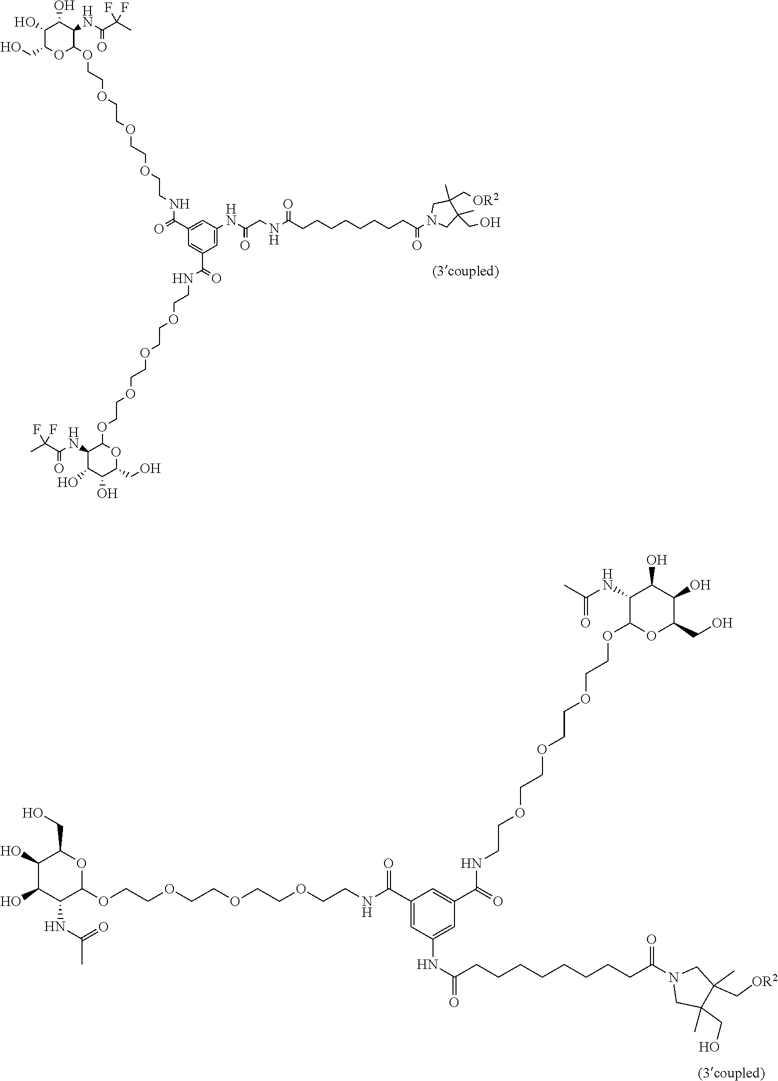

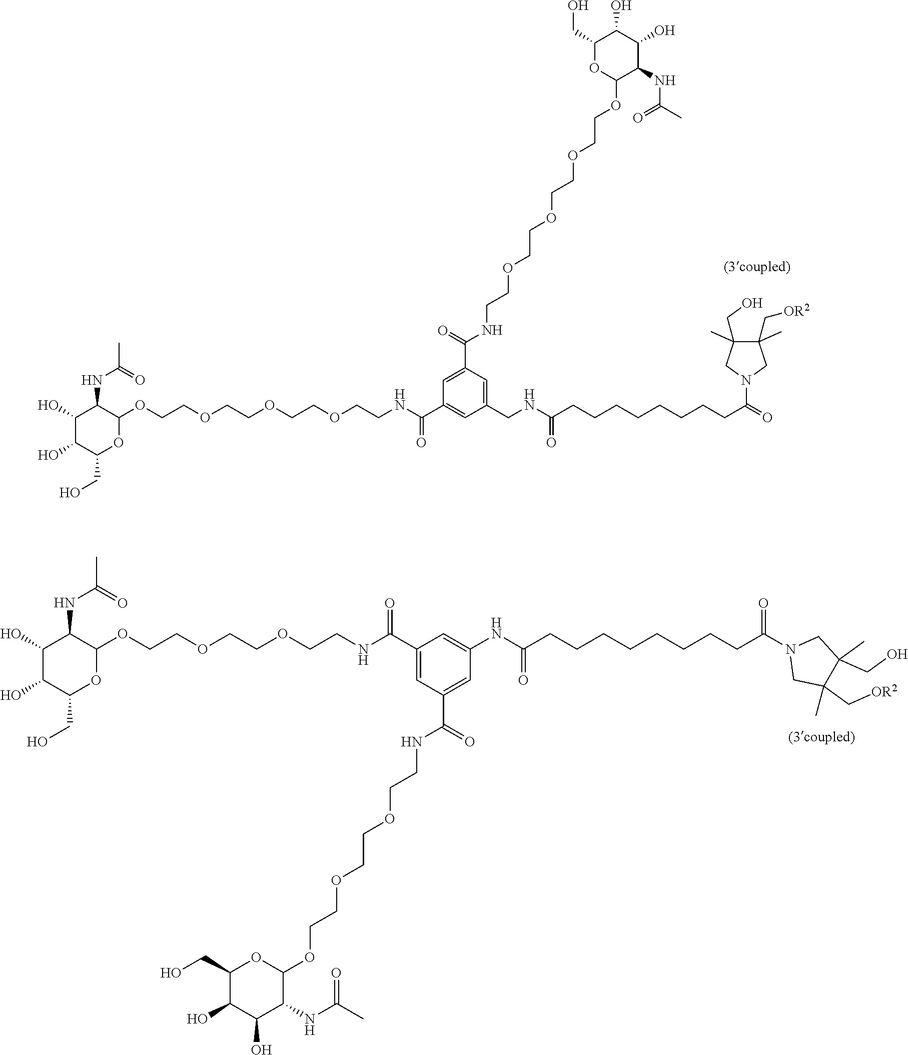

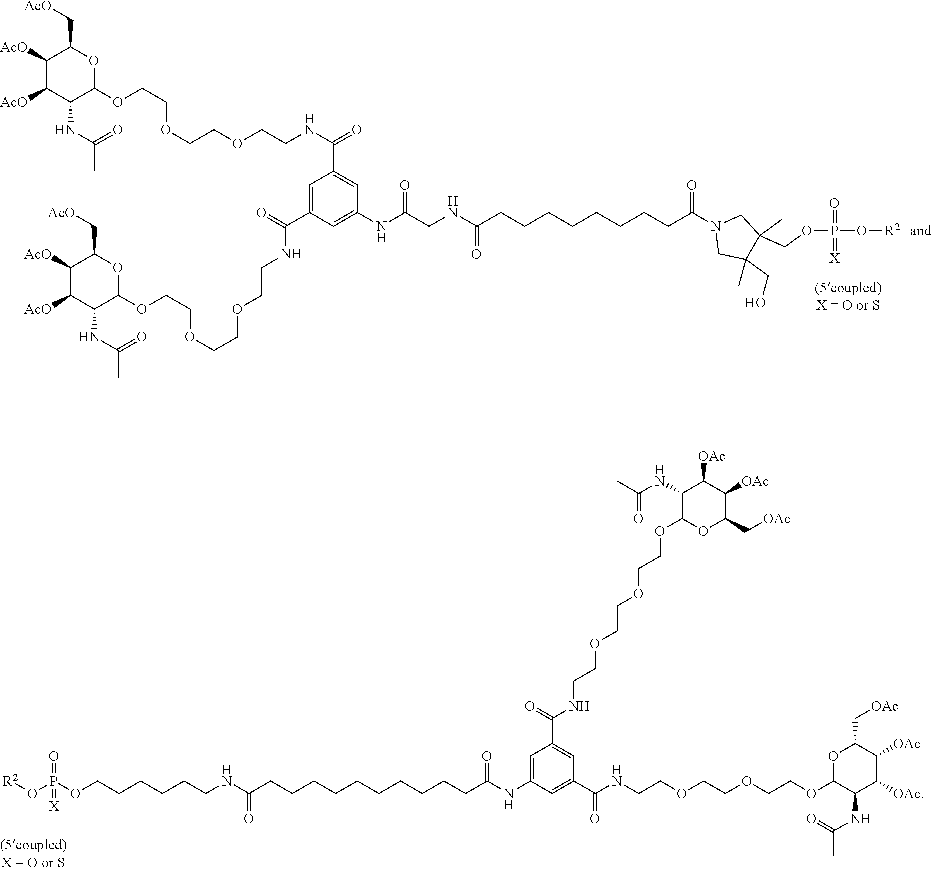

214. The method of any of claims 1-213, wherein the nucleic acid is an siRNA.

215. The method of claim 214 wherein the siRNA is linked to the remainder of the compound of formula (X), through the 3′-end of a sense strand.

216. The method of claim 214 wherein the siRNA is linked to the remainder of the compound of formula (X), through the 5′-end of a sense strand.

217. The method of claim 214 wherein the siRNA is linked to the remainder of the compound of formula (X), through the 3′-end of an antisense strand.

218. The method of claim 214 wherein the siRNA is linked to the remainder of the compound of formula (X), through the 5′-end of an antisense strand.

219. The method of any one of claims 214-218, wherein the siRNA is linked to the remainder of the compound of formula (X) through a phosphate on the siRNA.

220. A composition comprising:
a nucleic acid conjugate of formula (X) as described in any one of claims 1-219;
a membrane-destabilizing polymer as described in any one of claims 1-3 and 26-29; and
a pharmaceutically acceptable carrier.

221. The composition of claim 220 that is formulated for administration by injection.

222. The composition of claim 220 that is formulated for administration by subcutaneous injection.

223. A method for treating a disease characterized by overexpression of a polypeptide, comprising administering to a subject having the disease a therapeutically effective amount of (a) a nucleic acid conjugate of formula (X) as described in any one of claims 1-219, wherein C is a residue of siRNA that targets expression of the overexpressed polypeptide, and (b) a membrane-destabilizing polymer as described in any one of claims 1-3 and 26-29.

224. A method to deliver an siRNA to the liver of an animal, comprising administering to the animal, a membrane-destabilizing polymer as described in any one of claims 1-3 and 26-29; and a nucleic acid conjugate of formula (X) as described in any one of claims 1-219: wherein the targeting ligand is selected to promote hepatocyte-specific delivery of the conjugate, and wherein the nucleic acid is the siRNA.

225. A method to treat a hepatitis B viral infection in an animal, comprising administering to the animal, a) a nucleic acid conjugate of formula (X) as described in any one of claims 1-219, wherein the targeting ligand is selected to promote hepatocyte-specific delivery of the conjugate, and wherein the siRNA is useful to treat the hepatitis B viral infection; and b) a membrane-destabilizing polymer as described in any one of claims 1-3 and 26-29.

226. A kit comprising: 1) a membrane-destabilizing polymer as described in any one of claims 1-3 and 26-29; 2) a nucleic acid conjugate of Formula (X) as described in any one of claims 1-219, and 3) instructions for delivering a nucleic acid to a cell comprising contacting the cell with the nucleic acid conjugate and the membrane-destabilizing polymer.

227. A kit comprising: 1) a membrane-destabilizing polymer as described in any one of claims 1-3 and 26-29; 2) a nucleic acid conjugate of Formula (X) as described in any one of claims 1-219; and 3) instructions for delivering a nucleic acid to the cytosol of a target cell within a subject by administering the nucleic acid conjugate and the membrane-destabilizing polymer to the subject.

228. The kit of claim 227 wherein the membrane-destabilizing polymer is a polymer as described in any one of claims any one of claims 1-3 and 26-29.

229. A kit comprising: 1) a membrane-destabilizing polymer as described in any one of claims 1-3 and 26-29; 2) a nucleic acid conjugate of Formula (X) as described in any one of claims 1-219; and 3) instructions for administering the nucleic acid conjugate and the membrane-destabilizing polymer to an animal.

230. A membrane-destabilizing polymer as described in any one of claims 1-3 and 26-29; and a nucleic acid conjugate of Formula (X) as described in any one of claims 1-219; for use in medical therapy.

231. A nucleic acid conjugate of Formula (X) a as described in any one of claims 1-219; for the prophylactic or therapeutic treatment of a disease treatable with the nucleic acid, in combination with a membrane-destabilizing polymer as described in any one of claims 1-3 and 26-29.

232. The use of a nucleic acid conjugate of Formula (X) as described in any one of claims 1-219; to prepare a medicament for treating a disease treatable with the nucleic acid, in combination with a membrane-destabilizing polymer as described in any one of claims 1-3 and 26-29.

233. A nucleic acid conjugate of Formula (X) as described in any one of claims 1-219; that is associated non-covalently with a membrane-destabilizing polymer as described in any one of claims 1-3 and 26-29.

234. A nucleic acid conjugate of Formula (X) as described in any one of claims 1-219, that is partially or fully encapsulated by a micelle that comprises a plurality of membrane-destabilizing polymers, as described in any one of claims 1-3 and 26-29.

235. A nucleic acid conjugate of Formula (X) as described in any one of claims 1-219; that is partially encapsulated by a micelle that comprises a plurality of membrane-destabilizing polymers, as described in any one of claims 1-3 and 26-29.

236. A nucleic acid conjugate of Formula (X) as described in any one of claims 1-219; that is fully encapsulated by a micelle that comprises a plurality of membrane-destabilizing polymers, as described in any one of claims 1-3 and 26-29.

237. A pharmaceutical composition comprising a pharmaceutically acceptable carrier and a nucleic acid conjugate of Formula (X) as described in any one of claims 1-219; that is partially or fully encapsulated by a micelle that comprises a plurality of membrane-destabilizing polymers, as described in any one of claims 1-3 and 26-29.

238. The method of any one of claims 1-6 and 9-221, wherein the nucleic acid conjugate is administered after administration of the membrane-destabilizing polymer.
